(19) United States
(12) Patent Application Publication
(10) Pub. No.: US 2025/0040435 A1
(43) Pub. Date: Jan. 30, 2025

(54) ORGANIC LIGHT EMITTING DEVICE

(71) Applicant: LG CHEM, LTD., Seoul (KR)

(72) Inventors: Young Seok KIM, Daejeon (KR); Minjun KIM, Daejeon (KR); Sang Duk SUH, Daejeon (KR); Min Woo JUNG, Daejeon (KR); Donghee KIM, Daejeon (KR); Soyeon KIM, Daejeon (KR); Sunmi LEE, Daejeon (KR)

(21) Appl. No.: 18/760,803

(22) Filed: Jul. 1, 2024

(30) Foreign Application Priority Data
Jul. 3, 2023 (KR) 10-2023-0086027

(51) Int. Cl. H10K 85/60; C07D 307/77; C07D 307/91; C07D 405/04; C07D 405/10; C07D 405/14; C07D 409/14; C07F 7/08; C09K 11/06; H10K 50/12; H10K 85/40; H10K 101/00

(52) U.S. Cl. CPC H10K 85/6574; C07D 307/77; C07D 307/91; C07D 405/04; C07D 405/10; C07D 405/14; C07D 409/14; C07F 7/0812; C09K 11/06; H10K 85/40; H10K 85/615; H10K 85/626; H10K 85/636; H10K 85/654; H10K 85/6576; C09K 2211/1018; H10K 50/12; H10K 2101/90

(57) ABSTRACT

An organic light emitting device including an anode; a cathode opposite to the anode; and a light emitting layer interposed between the anode and the cathode, wherein the light emitting layer includes a first compound of Chemical Formula 1 and a second compound of Chemical Formula 2:

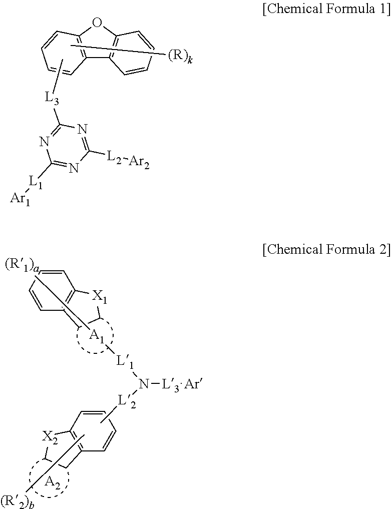

where Ar₁ and Ar₂ are each independently a substituted or unsubstituted C₆₋₆₀ aryl or C₂₋₆₀ heteroaryl containing at least one heteroatom selected among N, O and S; X₁ and X₂ are each independently O or S; A₁ and A₂ are each independently a naphthalene ring fused with an adjacent pentagonal ring; Ar′ is a substituted or unsubstituted C₆₋₆₀ aryl or C₂₋₆₀ heteroaryl containing at least one heteroatom selected among N, O and S; and the other substituents are as defined in the specification.

### CROSS-REFERENCE TO RELATED APPLICATIONS

This application claims priority to and the benefit of Korean Patent Application No. 10-2023-0086027 filed in the Korean Intellectual Property Office on Jul. 3, 2023, the entire contents of which are incorporated herein by reference.

### TECHNICAL FIELD

The present disclosure relates to an organic light emitting device.

### BACKGROUND ART

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic material used in the organic light emitting device as described above.

### PRIOR ART LITERATURE

### Patent Literature

(Patent Literature 0001) Korean Unexamined Patent Publication No. 10-2000-0051826

### DETAILED DESCRIPTION OF THE INVENTION

### Technical Problem

It is an object of the present disclosure to provide an organic light emitting device.

### Technical Solution

According to the present disclosure, there is provided the following organic light emitting device:

An organic light emitting device comprising:
an anode;
a cathode which is provided opposite to the anode; and
a light emitting layer which is interposed between the anode and the cathode,
wherein the light emitting layer includes a first compound represented by the following Chemical Formula 1, and a second compound represented by the following Chemical Formula 2:

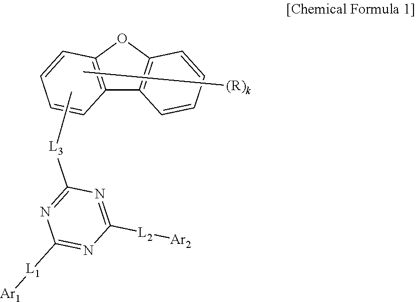

wherein in Chemical Formula 1,
L₁ to L₃ are each independently a single bond; or a substituted or unsubstituted C₆₋₆₀ arylene,
Ar₁ and Ar₂ are each independently a substituted or unsubstituted C₆₋₆₀ aryl; or a substituted or unsubstituted C₂₋₆₀ heteroaryl containing at least one heteroatom selected among N, O and S,
R is hydrogen; deuterium; halogen; cyano; or a substituted or unsubstituted C₁₋₆₀ alkyl,
k is an integer of 0 to 7,

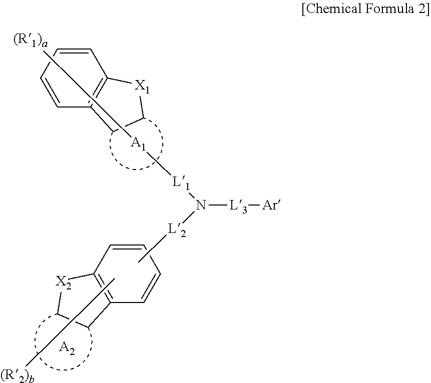

wherein in Chemical Formula 2,
X₁ and X₂ are each independently O or S,
A₁ and A₂ are each independently a naphthalene ring fused with an adjacent pentagonal ring,
L′₁ to L′₃ are each independently a single bond; or a substituted or unsubstituted C₆₋₆₀ arylene,
Ar′ is a substituted or unsubstituted C₆₋₆₀ aryl; or a substituted or unsubstituted C₂₋₆₀ heteroaryl containing at least one heteroatom selected among N, O and S,
R′₁ and R′₂ are each independently hydrogen; deuterium; halogen; cyano; or a substituted or unsubstituted C₁₋₆₀ alkyl, and
a and b are each independently an integer of 0 to 9.

### Advantageous Effects

The above-mentioned organic light emitting device includes two types of host compounds in the light emitting layer, and thus can improve efficiency, driving voltage and/or lifetime characteristics in the organic light emitting device.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

### DETAILED DESCRIPTION OF THE EMBODIMENTS

Hereinafter, embodiments of the present disclosure will be described in more detail to help understanding of the invention.

### Definition of Terms

In the present disclosure, the notation

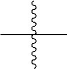

or [symbol] means a bond linked to another substituent group, and “D” means deuterium.

In the present disclosure, the term “substituted or unsubstituted” means being unsubstituted or substituted with one or more substituents selected from the group consisting of deuterium; a halogen group; a cyano group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heterocyclic group containing at least one of N, O and S atoms, or being unsubstituted or substituted with a substituent group to which two or more substituent groups of the above-exemplified substituent groups are linked. For example, “a substituent in which two or more substituents are linked” may be a biphenylyl group. Namely, a biphenylyl group may be an aryl group, or it may be interpreted as a substituent formed by linking two phenyl groups. In one example, the term “substituted or unsubstituted” may be understood as meaning “being unsubstituted or substituted with one or more substituents selected from the group consisting of deuterium, halogen, cyano, a silyl group, a C₁₋₁₀ alkyl, a C₁₋₁₀ alkoxy and a C₆₋₂₀ aryl”, or “being unsubstituted or substituted with one or more substituents selected from the group consisting of deuterium, halogen, cyano, methyl, ethyl, phenyl, biphenylyl and naphthyl”. Further, the term “substituted with one or more substituents” as used herein may be understood as meaning “being substituted with mono to the maximum number of substitutable hydrogens”. Alternatively, the term “substituted with one or more substituents” as used herein may be understood as meaning “being substituted with 1 to 5 substituents”, or “being substituted with one or two substituents”.

In the present disclosure, the carbon number of a carbonyl group is not particularly limited, but is preferably 1 to 40. Specifically, the carbonyl group may be a substituent having the following structural formulas, but is not limited thereto.

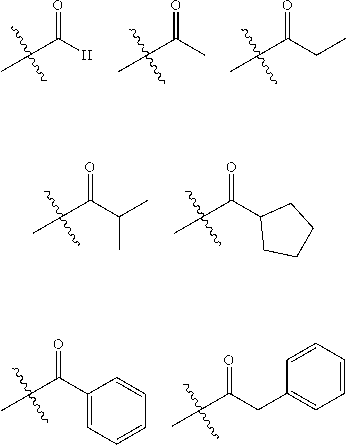

In the present disclosure, an ester group may have a structure in which oxygen of the ester group may be substituted by a straight-chain, branched-chain, or cyclic alkyl group having 1 to 25 carbon atoms, or an aryl group having 6 to 25 carbon atoms. Specifically, the ester group may be a substituent having the following structural formulas, but is not limited thereto.

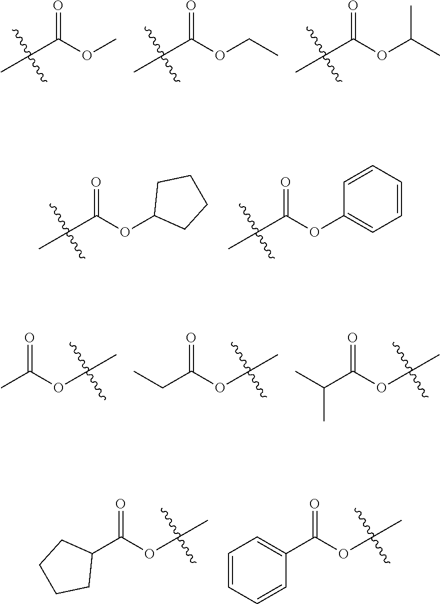

In the present disclosure, the carbon number of an imide group is not particularly limited, but is preferably 1 to 25. Specifically, the imide group may be a substituent group having the following structural formulas, but is not limited thereto.

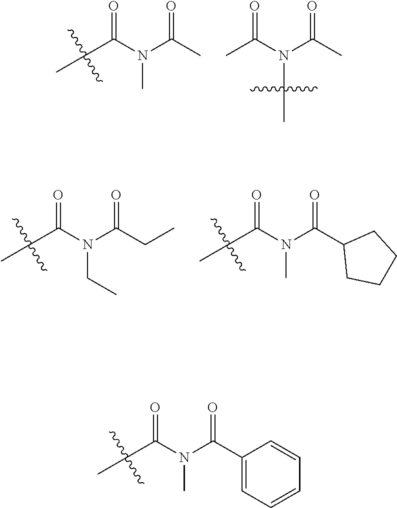

In the present disclosure, a silyl group means —Si(Z₁)(Z₂)(Z₃), wherein Z₁, Z₂ and Z₃ are each independently hydrogen, deuterium, a substituted or unsubstituted C₁₋₆₀ alkyl, a substituted or unsubstituted C₁₋₆₀ haloalkyl, a substituted or unsubstituted C₂₋₆₀ alkenyl, a substituted or unsubstituted C₂₋₆₀ haloalkenyl, or a substituted or unsubstituted C₆₋₆₀ aryl. According to one embodiment, Z₁, Z₂ and Z₃ may be each independently hydrogen, deuterium, a substituted or unsubstituted C₁₋₁₀ alkyl, a substituted or unsubstituted C₁₋₁₀ haloalkyl, or a substituted or unsubstituted C₆₋₂₀ aryl. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluoro, chloro, bromo, or iodo.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, 1-ethyl-propyl, 1,1-dimethylpropyl, hexyl, n-hexyl, 1-methylpentyl, 2-methylpentyl, 4-methyl-2-pentyl, 3,3-dimethylbutyl, 2-ethylbutyl, heptyl, n-heptyl, isohexyl, 1-methylhexyl, 2-methylhexyl, 3-methylhexyl, 4-methylhexyl, 5-methylhexyl, cyclopentylmethyl, cyclohexylmethyl, octyl, n-octyl, tert-octyl, 1-methylheptyl, 2-ethylhexyl, 2,4,4-trimethyl-1-pentyl, 2,4,4-trimethyl-2-pentyl, 2-propylpentyl, n-nonyl, 2,2-dimethylheptyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, the alicyclic group means a monovalent substituent derived from a saturated or unsaturated hydrocarbon ring compound that contains only carbon as a ring-forming atom, but does not have aromaticity, which is understood to encompass both monocyclic and fused polycyclic compounds. According to one embodiment, the carbon number of the alicyclic group is 3 to 60. According to another embodiment, the carbon number of the alicyclic group is 3 to 30. According to another embodiment, the carbon number of the alicyclic group is 3 to 20. Examples of the alicyclic group include a monocyclic group such as a cycloalkyl group, a bridged hydrocarbon group, a spiro hydrocarbon group, a substituent derived from hydrogenated derivatives of aromatic hydrocarbon compound.

Specifically, examples of the cycloalkyl group include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

Further, examples of the bridged hydrocarbon group include bicyclo[1.1.0]butyl, bicyclo[2.2.1]heptyl, bicyclo[4.2.0]octa-1,3,5-trienyl, adamantyl, decalinyl, and the like, but are not limited thereto.

Further, examples of the spiro hydrocarbon group include spiro[3.4]octyl, spiro[5.5]undecanyl, and the like, but are not limited thereto.

Further, a substituent derived from a hydrogenated derivative of the aromatic hydrocarbon compound means a substituent derived from a monocyclic or polycyclic aromatic hydrocarbon compound in which a part of the compound is hydrogenated. Examples of such a substituent include 1H-indenyl, 2H-indenyl, 4H-indenyl, 2,3-dihydro-1H-indenyl, 1,4-dihydronaphthalenyl, 1,2,3,4-tetrahydronaphthalenyl, 6,7,8,9-tetrahydro-5H-benzo[7]annulenyl, 6,7-dihydro-5H-benzocycloheptenyl, and the like, but are not limited thereto.

In the present disclosure, an aryl group is understood to mean a substituent derived from a monocyclic or fused polycyclic compound containing only carbon as a ring-forming atom and also having aromaticity, and the carbon number thereof is not particularly limited, but is preferably 6 to 60. According to one embodiment, the carbon number of the aryl group is 6 to 30. According to one embodiment, the carbon number of the aryl group is 6 to 20. The aryl group may be a phenyl group, a biphenylyl group, a terphenylyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, a fluorenyl group, or the like, but is not limited thereto.

In the present disclosure, the fluorenyl group may be substituted, and two substituent groups may be linked with each other to form a spiro structure.

In the case where the fluorenyl group is substituted,

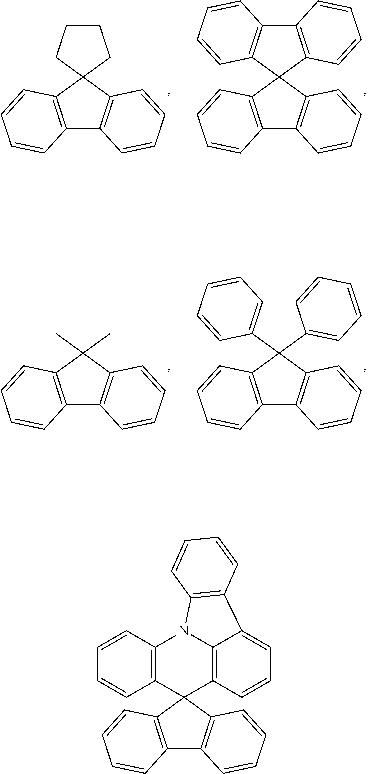

and the like can be formed. However, the structure is not limited thereto.

In the present disclosure, a heterocyclic group means a monovalent substituent derived from a monocyclic or fused polycyclic compound that further contains at least one heteroatom selected among O, N, Si, and S in addition to carbon as a ring-forming atom, and is understood to encompass both substituents with aromaticity and substituents without aromaticity. According to one embodiment, the carbon number of the heterocyclic group is 2 to 60 carbon atoms. According to another embodiment, the carbon number of the heterocyclic group is 2 to 30. According to another embodiment, the carbon number of the heterocyclic group is 2 to 20. Examples of such a heterocyclic group include a heteroaryl group, a substituent derived from a hydrogenated derivative of the heteroaromatic compound, and the like.

Specifically, the heteroaryl group means a substituent derived from a monocyclic or fused polycyclic compound which further contains at least one heteroatom selected among N, O and S in addition to carbon as a ring forming atom, and refers to a substituent having aromaticity. According to one embodiment, the carbon number of the heteroaryl group is 2 to 60. According to another embodiment, the carbon number of the heteroaryl group is 2 to 30. According to another embodiment, the carbon number of the heteroaryl group is 2 to 20. According to another embodiment, the carbon number of the heteroaryl group is 2 to 12. According to another embodiment, the carbon number of the heteroaryl group is 2 to 10. According to another embodiment, the carbon number of the heteroaryl group is 2 to 8. Examples of the heteroaryl group include a thiophenyl group, a furanyl group, a pyrrole group, an imidazolyl group, a thiazolyl group, an oxazolyl group, an oxadiazolyl group, a triazolyl group, a pyridinyl group, a bipyridinyl group, a pyrimidinyl group, a triazinyl group, an acridinyl group, a pyridazinyl group, a pyrazinyl group, a quinolinyl group, a quinazolinyl group, a quinoxalinyl group, a phthalazinyl group, a pyridopyrimidinyl group, a pyridopyrazinyl group, an isoquinolinyl group, an indolyl group, a carbazolyl group, a benzoxazolyl group, a benzoimidazolyl group, a benzothiazolyl group, a benzocarbazolyl group, a benzothiophenyl group, a dibenzothiophenyl group, a benzofuranyl group, a dibenzofuranyl group, a phenanthrolinyl group, an isoxazolyl group, a thiadiazolyl group, a phenothiazinyl group, and the like, but are not limited thereto.

Further, a substituent derived from a hydrogenated derivative of a heteroaromatic compound means a substituent derived from a monocyclic or polycyclic heteroaromatic compound in which a part of the unsaturated bond of the compound is hydrogenated. Examples of such substituents include 1,3-dihydroisobenzofuranyl, 2,3-dihydrobenzofuranyl, 1,3-dihydrobenzo[c]thiophenyl, 2,3-dihydro[b]thiophenyl, and the like, but are not limited thereto.

In the present disclosure, the aryl group in the aralkyl group, the aralkenyl group, the alkylaryl group, the arylamine group and the arylsilyl group is the same as the examples of the aryl group as defined above. In the present disclosure, the alkyl group in the aralkyl group, the alkylaryl group and the alkylamine group is the same as the examples of the alkyl group as defined above. In the present disclosure, the heteroaryl in the heteroarylamine can be applied to the description of the heteroaryl as defined above. In the present disclosure, the alkenyl group in the aralkenyl group is the same as the examples of the alkenyl group as defined above. In the present disclosure, the description of the aryl group as defined above may be applied except that the arylene is a divalent group. In the present disclosure, the description of the heteroaryl as defined above can be applied except that the heteroarylene is a divalent group. In the present disclosure, the description of the aryl group or cycloalkyl group as defined above can be applied except that the hydrocarbon ring is not a monovalent group but formed by combining two substituent groups. In the present disclosure, the description of the heteroaryl as defined above can be applied, except that the heterocycle is not a monovalent group but formed by combining two substituent groups.

In the present disclosure, the term “deuterated or substituted with deuterium” means that at least one of the substitutable hydrogens in a compound, a divalent linking group, or a monovalent substituent has been substituted with deuterium.

Further, the term “unsubstituted or substituted with deuterium” or “substituted or unsubstituted with deuterium” means that “mono to the maximum number of unsubstituted or substitutable hydrogens have been substituted with deuterium.” In one example, the term “phenanthryl unsubstituted or substituted with deuterium” may be understood as meaning “phenanthryl unsubstituted or substituted with 1 to 9 deuterium atoms”, considering that the maximum number of hydrogen that can be substituted with deuterium in the phenanthryl structure is 9.

Further, “deuterated structure” means to include compounds, divalent linking groups, or monovalent substituents of all structures in which at least one hydrogen is substituted with deuterium. As an example, the deuterated structure of phenyl can be understood to refer to monovalent substituents of all structures in which at least one substitutable hydrogen in the phenyl group is substituted with deuterium, as follows.

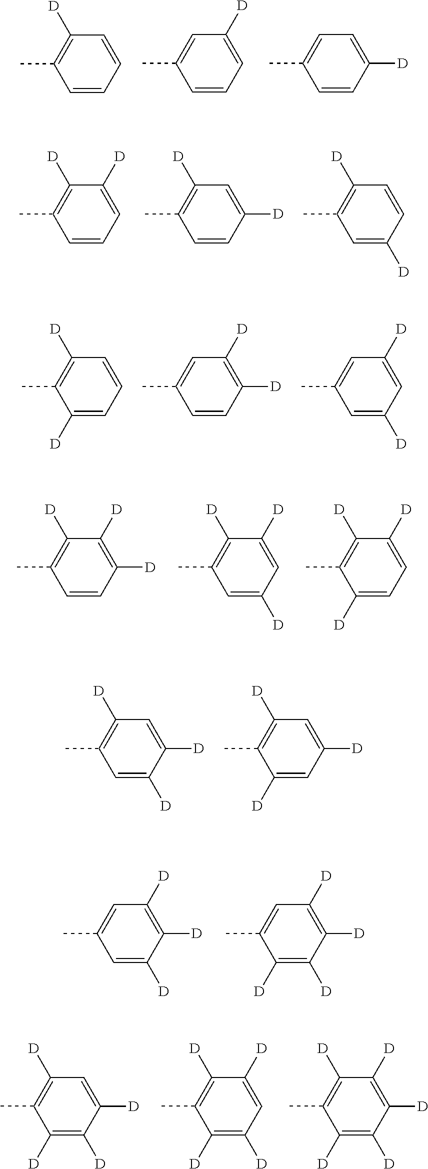

In addition, the “deuterium substitution rate” or “degree of deuteration” of a compound means that the ratio of the number of substituted deuterium atoms to the total number of hydrogen atoms (the sum of the number of hydrogen atoms substitutable with deuterium and the number of substituted deuterium atoms in a compound) that can exist in the compound is calculated as a percentage. Therefore, when the “deuterium substitution rate” or “degree of deuteration” of a compound is “K %”, it means that K % of the hydrogen atoms substitutable with deuterium in the compound are substituted with deuterium.

At this time, the “deuterium substitution rate” or “degree of deuteration” can be determined according to a commonly known method using MALDI-TOF MS (Matrix-Assisted Laser Desorption/Ionization Time-of-Flight Mass Spectrometer), a nuclear magnetic resonance spectroscopy (¹H NMR), TLC/MS (Thin-Layer Chromatography/Mass Spectrometry), GO/MS (Gas Chromatography/Mass Spectrometry), or the like. More specifically, when using MALDI-TOF MS, the “deuterium substitution rate” or “degree of deuteration” may be obtained by determining the number of substituted deuterium in the compound through MALDI-TOF MS analysis, and then calculating the ratio of the number of substituted deuterium to the total number of hydrogen atoms that can exist in the compound as a percentage.

Provided herein is an organic light emitting device comprising: an anode; a cathode which is provided opposite to the anode; and a light emitting layer which is interposed between the anode and the cathode, wherein the light emitting layer includes the first compound represented by Chemical Formula 1, and the second compound represented by Chemical Formula 2.

The organic light emitting device according to the present disclosure simultaneously contains two types of compounds having a specific structure as host materials in the light emitting layer, and thus can improve efficiency, driving voltage, and/or lifetime characteristics in the organic light emitting device.

Hereinafter, the present disclosure will be described in detail for each configuration.

### Anode and Cathode

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

### Hole Injection Layer

The organic light emitting device according to the present disclosure may include a hole injection layer between an anode and a hole transport layer described hereinafter, if necessary.

The hole injection layer is a layer that is located on the anode and injects holes from the anode, which includes a hole injection material. The hole injection material is preferably a compound which has a capability of transporting the holes, a hole injection effect in the anode and an excellent hole injection effect to the light emitting layer or the light emitting material, prevents movement of an exciton generated in the light emitting layer to the electron injection layer or the electron injection material, and has an excellent thin film forming ability. In particular, it is suitable that a HOMO (highest occupied molecular orbital) of the hole injection material is between the work function of the anode material and a HOMO of a peripheral organic material layer.

Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

### Hole Transport Layer

The organic light emitting device according to the present disclosure may include a hole transport layer between the anode and the light emitting layer. The hole transport layer is a layer that receives holes from an anode or a hole injection layer formed on the anode and transports the holes to the light emitting layer, which includes a hole transport material. The hole transport material is suitably a material having large mobility to the holes, which may receive holes from the anode or the hole injection layer and transfer the holes to the light emitting layer. Specific examples thereof may include an arylamine-based organic material, a conductive polymer, a block copolymer in which a conjugate portion and a non-conjugate portion are present together, and the like, but are not limited thereto.

### Electron Blocking Layer

The organic light emitting device according to the present disclosure may include an electron blocking layer between the hole transport layer and the light emitting layer, if necessary. The electron blocking layer refers to a layer which is formed on the hole transport layer, preferably provided in contact with the light emitting layer, and serves to adjust the hole mobility, prevent excessive movement of electrons, and increase the probability of hole-electron coupling, thereby improving the efficiency of the organic light emitting device. The electron blocking layer includes an electron blocking material, and examples of such an electron blocking material may include an arylamine-based organic material or the like, but is not limited thereto.

### Light Emitting Layer

The organic light emitting device according to the present disclosure may include a light emitting layer between an anode and a cathode, and the light emitting layer includes the first compound and the second compound as the host material. Specifically, the first compound can function as an N-type host material in which an electron transport capability is superior to a hole transport capability, and the second compound can function as a P-type host material in which a hole transport capability is superior to an electron transport capability, and thus can properly maintain the ratio of holes to electrons in the light emitting layer. Accordingly, excitons emit light evenly throughout the light emitting layer, so that the light emitting efficiency and lifetime characteristics of the organic light emitting device can be improved at the same time.

Hereinafter, the first compound and the second compound will be sequentially described.

### (First Compound)

The first compound is represented by the Chemical Formula 1. Specifically, the first compound is a compound in which one triazinyl group is substituted on dibenzofuran, has excellent electron transport ability and thus can efficiently transfer electrons to the dopant material, thereby increasing the recombination probabilities of electron-hole in the light emitting layer.

Meanwhile, when the bonding position of L₃ in Chemical Formula 1 is to be specifically represented, it may be represented by the following Chemical Formula 1′:

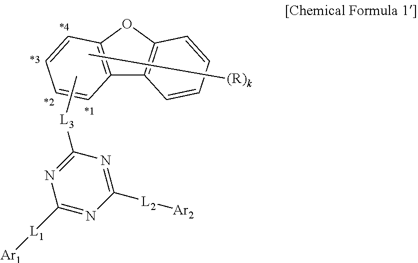

wherein in Chemical Formula 1′,
L₃ is linked to any one of carbon at position *1, carbon at position *2, carbon at position *3, and carbon at position *4, and
L₁ to L₃, Ar₁, Ar₂, R and k are as defined in Chemical Formula 1.

For example, the Chemical Formula 1 may be represented by the following Chemical Formula 1-1:

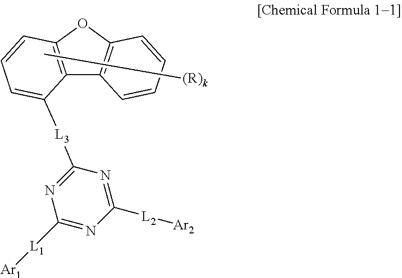

wherein in Chemical Formula 1-1,
L₁ to L₃, Ar₁, Ar₂, R and k are as defined in Chemical Formula 1.

In one embodiment, L₁ and L₂ may be each independently a single bond, or a substituted or unsubstituted C₆₋₂₀ arylene.

In another embodiment, L₁ and L₂ may be each independently a single bond; or a C₆₋₂₀ arylene which is unsubstituted, or substituted with one or more substituents selected from the group consisting of deuterium, phenyl and naphthyl.

In yet another embodiment, L₁ and L₂ may be each independently a single bond, or a substituted or unsubstituted C₆₋₁₂ arylene.

In yet another embodiment, L₁ and L₂ may be each independently a single bond, a substituted or unsubstituted phenylene, a substituted or unsubstituted biphenyldiyl, or a substituted or unsubstituted naphthylene.

In yet another embodiment, L₁ and L₂ may be each independently a single bond, phenylene, biphenyldiyl, or naphthylene,
wherein the phenylene, biphenyldiyl and naphthylene may be unsubstituted or substituted with deuterium.

For example, L₁ and L₂ may be each independently a single bond, or any one selected from the group consisting of the following formulas and the deuterated structure thereof:

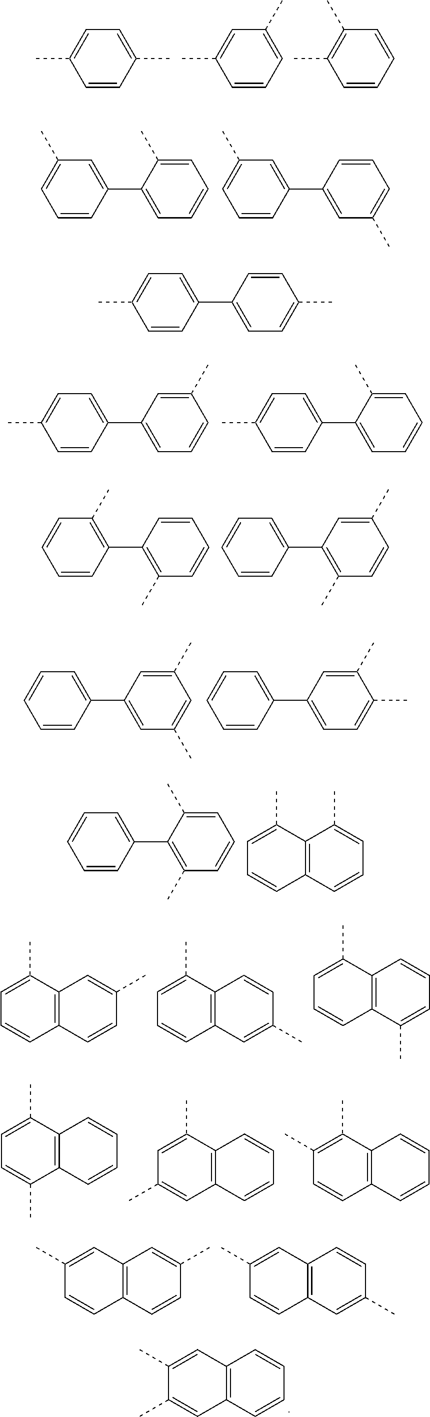

Further, L₁ and L₂ may be identical to each other. Alternatively, L₁ and L₂ may be different from each other.

For example, both L₁ and L₂ are a single bond; or
one of L₁ and L₂ is a single bond, and the other one may be phenylene substituted or unsubstituted with deuterium, or naphthylene substituted or unsubstituted with deuterium.

Further, in one embodiment, L₃ may be a single bond, or a substituted or unsubstituted C₆₋₂₀ arylene.

In other embodiments, L₃ may be a single bond; or a C₆₋₂₀ arylene which is unsubstituted, or substituted with one or more substituents selected from the group consisting of deuterium, phenyl and naphthyl.

In yet another embodiment, L₃ may be a single bond, a substituted or unsubstituted phenylene, or a substituted or unsubstituted naphthylene.

For example, L₃ may be a single bond, or any one of the divalent linking groups represented by the following Chemical Formulas 4a to 4m:

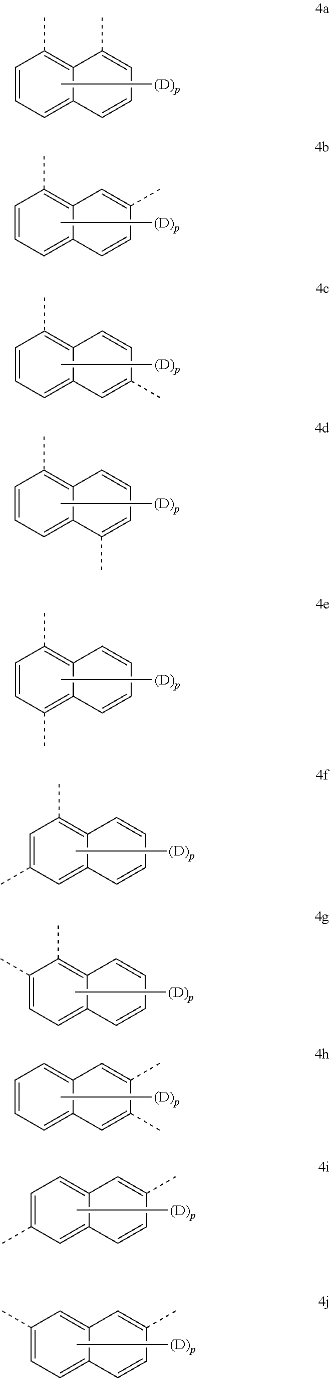

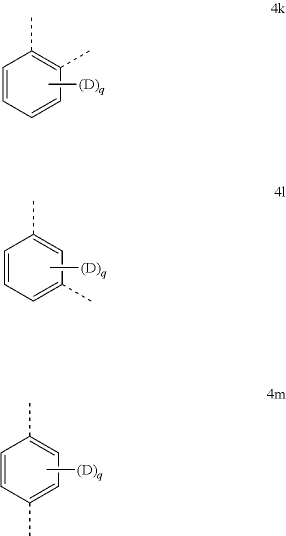

wherein in Chemical Formulas 4a to 4m,
D means deuterium,
p is an integer of 0 to 6, and
q is an integer of 0 to 4.
In other words, in Chemical Formulas 4a to 4m,
p is 0, 1, 2, 3, 4, 5, or 6, and
q is 0, 1, 2, 3, or 4.

Further, in one embodiment, Ar and Ar₂ may be a substituted or unsubstituted C₆₋₂₀ aryl.

In other embodiments, Ar₁ and Ar₂ may be each independently a C₆₋₂₀ aryl which is unsubstituted or substituted with one or more substituents selected from the group consisting of deuterium, a C₁₋₁₀ alkyl substituted or unsubstituted with deuterium, a C₆₋₂₀ aryl substituted or unsubstituted with deuterium, —Si(C₁₋₁₀ alkyl substituted or unsubstituted with deuterium)₃ and —Si(C₆-20 aryl substituted or unsubstituted with deuterium)₃.

Wherein the three substituents bonded to Si in —Si(C₁₋₁₀ alkyl substituted or unsubstituted with deuterium)₃ and —Si(C₆₋₂₀ aryl substituted or unsubstituted with deuterium)₃ may be the same as or different from each other.

Specifically, for example, Ar₁ and Ar₂ may be each independently phenyl, biphenylyl, terphenylyl, naphthyl, phenanthryl, dibenzofuranyl, dibenzothiophenyl, benzonaphthofuranyl, or benzonaphthothiophenyl,
wherein Ar₁ and Ar₂ may be unsubstituted, or substituted with one or more substituents selected from the group consisting of deuterium, halogen, cyano, phenyl substituted or unsubstituted with deuterium, biphenylyl substituted or unsubstituted with deuterium, naphthyl substituted or unsubstituted with deuterium, and —Si(phenyl substituted or unsubstituted with deuterium)₃.

For example, Ar₁ and Ar₂ may be each independently any one selected from the group consisting of the following formulas and the deuterated structure thereof:

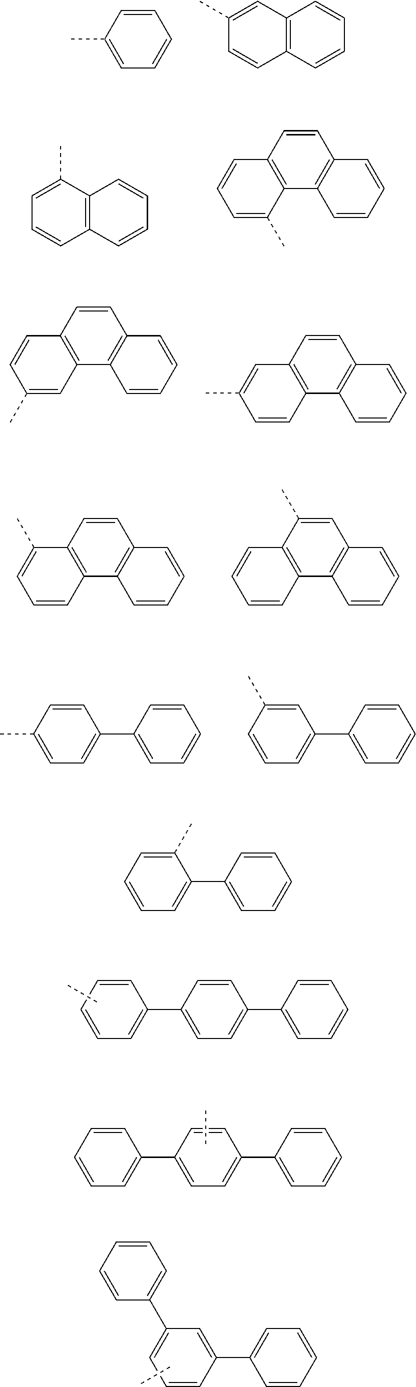

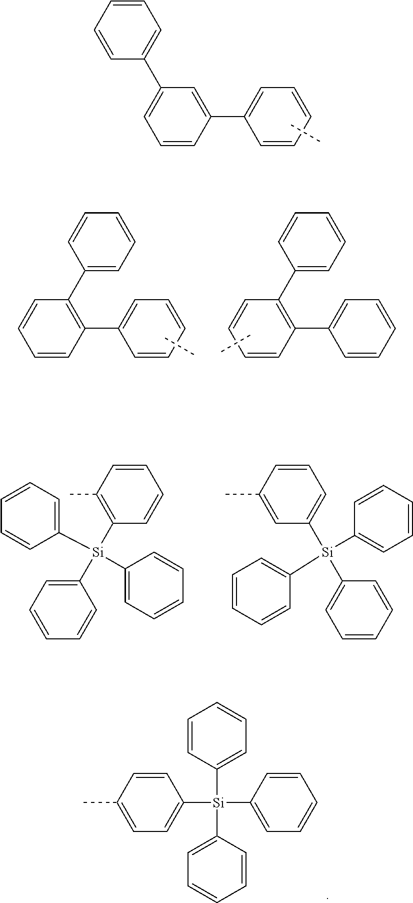

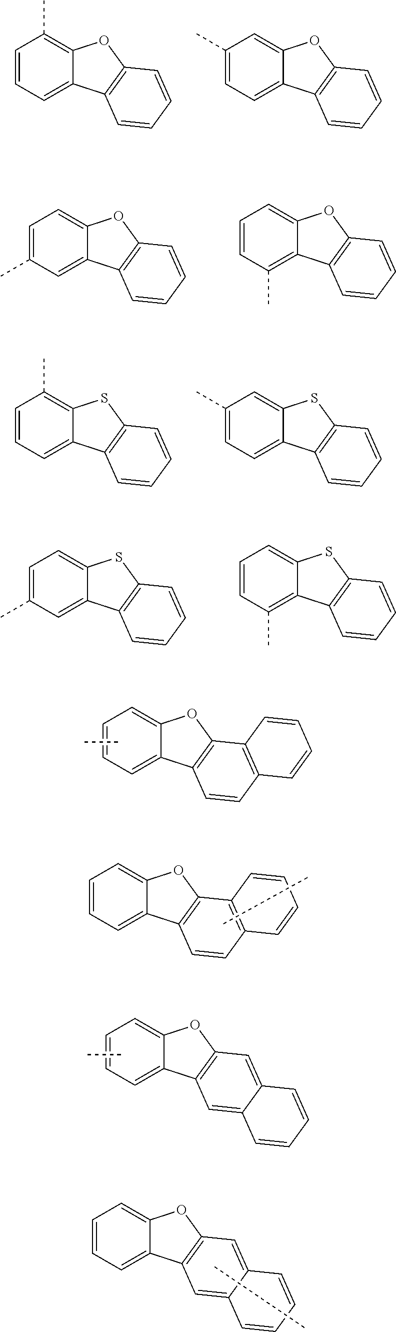

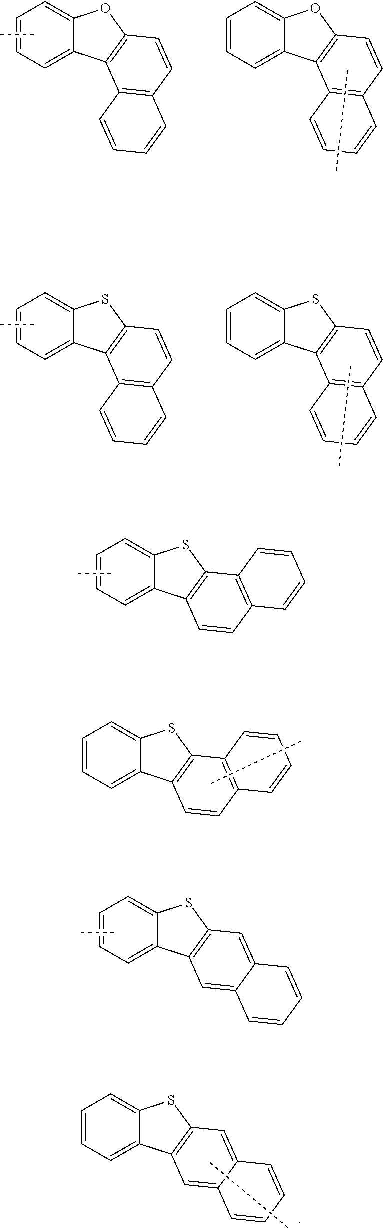

In one embodiment, Ar₁ and Ar₂ may be identical to each other. Alternatively, Ar₁ and Ar₂ may be different from each other.

In other embodiments, L₁-Ar₁ and L₂-Ar₂ may be identical to each other.

Alternatively, L₁-Ar₁ and L₂-Ar₂ may be different from each other.

Further, in one embodiment, R may be deuterium, halogen, methyl, or ethyl.

Wherein, k means the number of R, and when k is 2 or more, two or more R may be the same as or different from each other. Specifically, k is 0, 1, 2, 3, 4, 5, 6, or 7.

For example, k is 0; or

K is 1 or more, and R may be deuterium.

Further, the first compound may not contain deuterium, or may contain at least one deuterium.

When the first compound contains deuterium, the deuterium substitution rate of the first compound may be 1% to 100%. Specifically, the deuterium substitution rate of the first compound may be 5% or more, 10% or more, 20% or more, 30% or more, 40% or more, 50% or more, 60% or more, 70% or more, 75% or more, 80% or more, or 90% or more, and 100% or less.

In one embodiment, the first compound may not contain deuterium, or may contain 1 to 50 deuterium atoms. More specifically, the first compound may not contain deuterium, or may contain 1 or more, 2 or more, 3 or more, 4 or more, 5 or more, 6 or more, 7 or more, 8 or more, or 9 or more, and 50 or less, 40 or less, 30 or less, 28 or less, 26 or less, 24 or less, 22 or less, 20 or less, 18 or less, 16 or less, 14 or less, 13 or less, 12 or less, 11 or less, or 10 or less deuterium atoms.

In this case, when the number of deuterium substitution of the first compound is to be represented, it may be represented by the following Chemical Formula 1D:

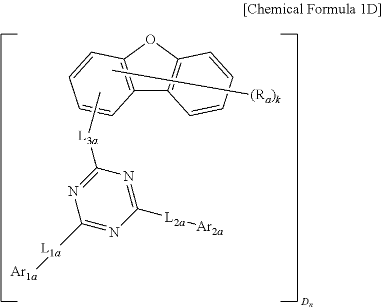

wherein in Chemical Formula 1D,
Dn means that n hydrogens have been replaced by deuterium,
wherein n is an integer of 0 or more,
L₁ₐ to L₃ₐ, Ar₁ₐ, Ar₂ₐ and Rₐ mean L₁ to L₃, Ar₁, Ar₂ and R substituents which are not substituted with deuterium, respectively.

Meanwhile, representative examples of the first compound are as follows:

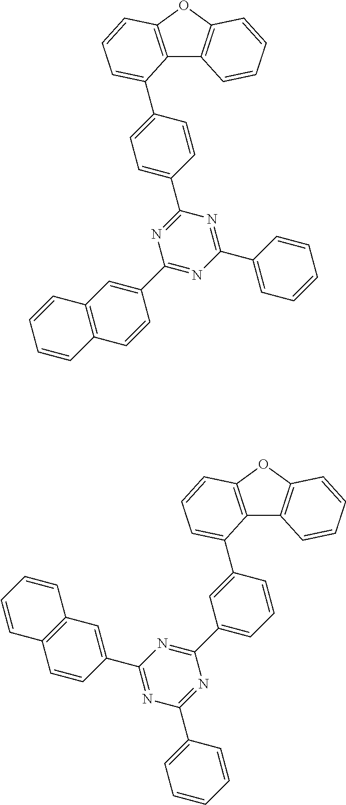 [SMILES: c1ccc(-c2nc(-c3ccc(-c4cccc5oc6ccccc6c45)cc3)nc(-c3ccc4ccccc4c3)n2)cc1 ; c1ccc(-c2nc(-c3cccc(-c4cccc5oc6ccccc6c45)c3)nc(-c3ccc4ccccc4c3)n2)cc1]

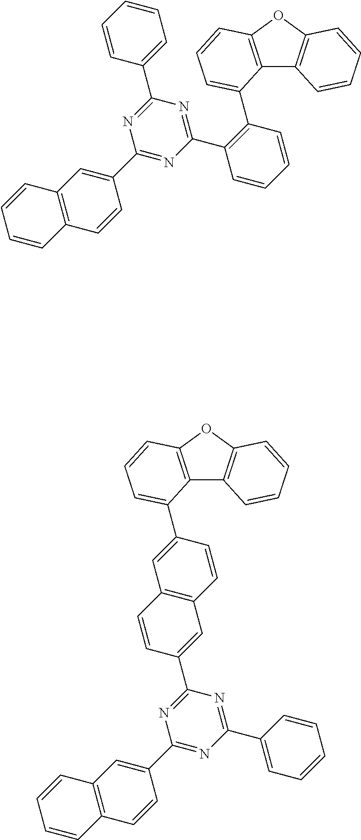 [SMILES: c1ccc(-c2nc(-c3ccc4ccccc4c3)nc(-c3ccccc3-c3cccc4oc5ccccc5c34)n2)cc1 ; c1ccc(-c2nc(-c3ccc4ccccc4c3)nc(-c3ccc4cc(-c5cccc6oc7ccccc7c56)ccc4c3)n2)cc1]

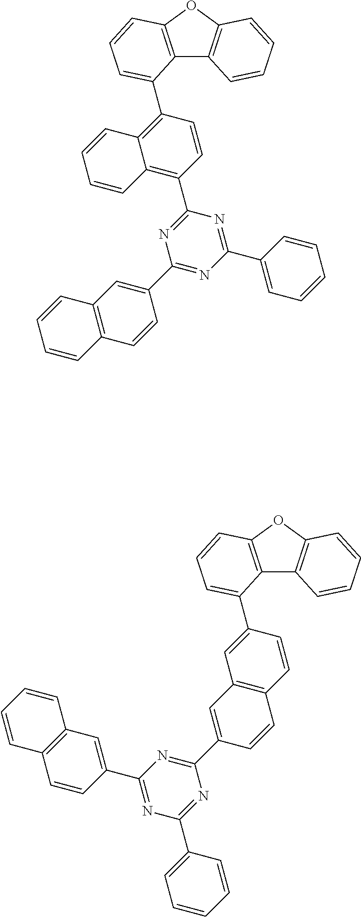 [SMILES: c1ccc(-c2nc(-c3ccc4ccccc4c3)nc(-c3ccc(-c4cccc5oc6ccccc6c45)c4ccccc34)n2)cc1 ; c1ccc(-c2nc(-c3ccc4ccccc4c3)nc(-c3ccc4ccc(-c5cccc6oc7ccccc7c56)cc4c3)n2)cc1]

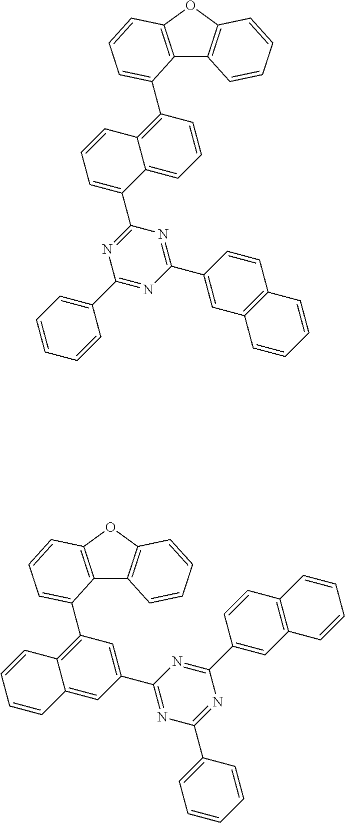 [SMILES: c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1]

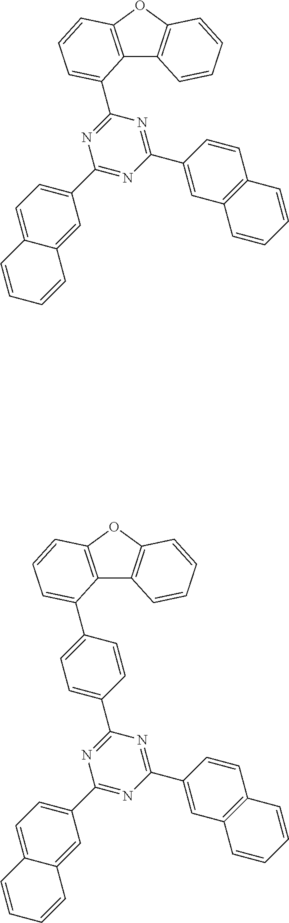 [SMILES: c1ccc2cc(-c3nc(-c4ccc5ccccc5c4)nc(-c4cccc5oc6ccccc6c45)n3)ccc2c1 ; c1ccc2cc(-c3nc(-c4ccc(-c5cccc6oc7ccccc7c56)cc4)nc(-c4ccc5ccccc5c4)n3)ccc2c1]

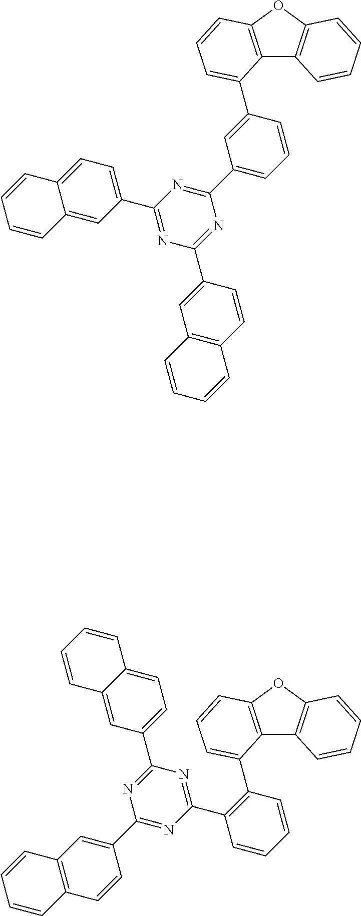 [SMILES: c1cc(-c2nc(-c3ccc4ccccc4c3)nc(-c3ccc4ccccc4c3)n2)cc(-c2cccc3oc4ccccc4c23)c1 ; c1ccc(-c2cccc3oc4ccccc4c23)c(-c2nc(-c3ccc4ccccc4c3)nc(-c3ccc4ccccc4c3)n2)c1]

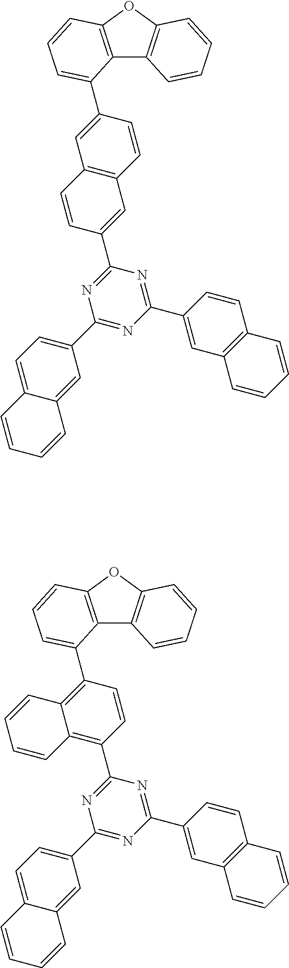 [SMILES: c1ccc2cc(-c3nc(-c4ccc5ccccc5c4)nc(-c4ccc5cc(-c6cccc7oc8ccccc8c67)ccc5c4)n3)ccc2c1 ; c1ccc2cc(-c3nc(-c4ccc5ccccc5c4)nc(-c4ccc(-c5cccc6oc7ccccc7c56)c5ccccc45)n3)ccc2c1]

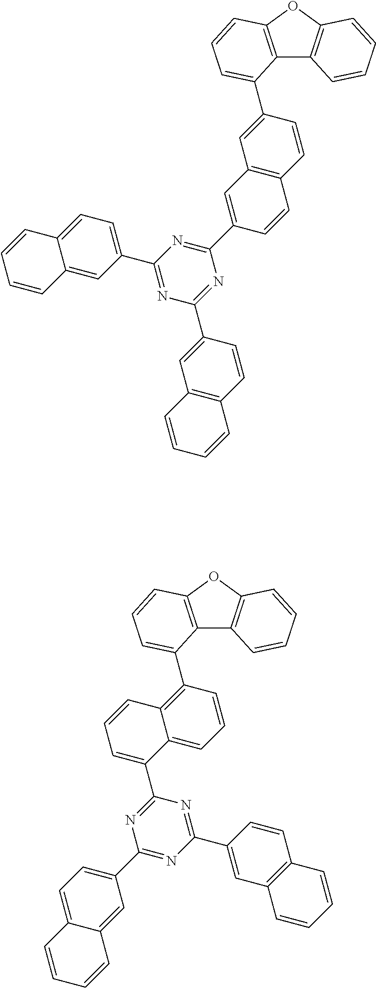 [SMILES: c1ccc2cc(-c3nc(-c4ccc5ccccc5c4)nc(-c4ccc5ccc(-c6cccc7oc8ccccc8c67)cc5c4)n3)ccc2c1 ; c1ccc2cc(-c3nc(-c4ccc5ccccc5c4)nc(-c4cccc5c(-c6cccc7oc8ccccc8c67)cccc45)n3)ccc2c1]

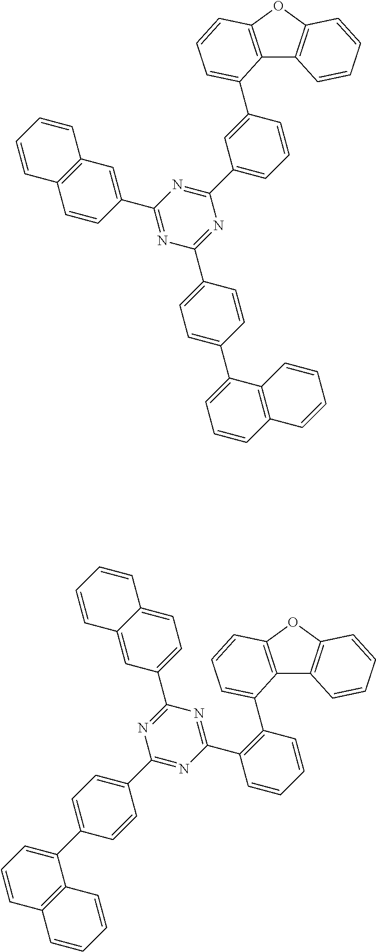 [SMILES: c1cc(-c2nc(-c3ccc(-c4cccc5ccccc45)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4cccc5ccccc45)cc3)nc(-c3ccc4ccccc4c3)n2)c1]

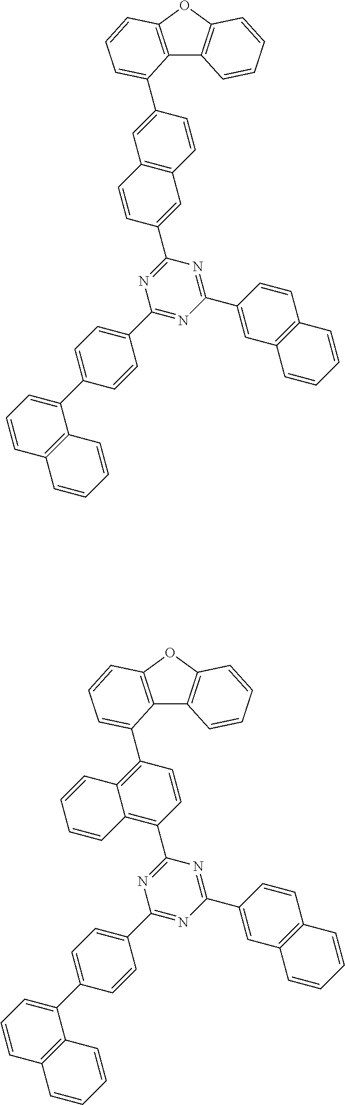 [SMILES: c1ccc2cc(-c3nc(-c4ccc(-c5cccc6ccccc56)cc4)nc(-c4ccc5cc(-c6cccc7oc8ccccc8c67)ccc5c4)n3)ccc2c1 ; c1ccc2cc(-c3nc(-c4ccc(-c5cccc6ccccc56)cc4)nc(-c4ccc(-c5cccc6oc7ccccc7c56)c5ccccc45)n3)ccc2c1]

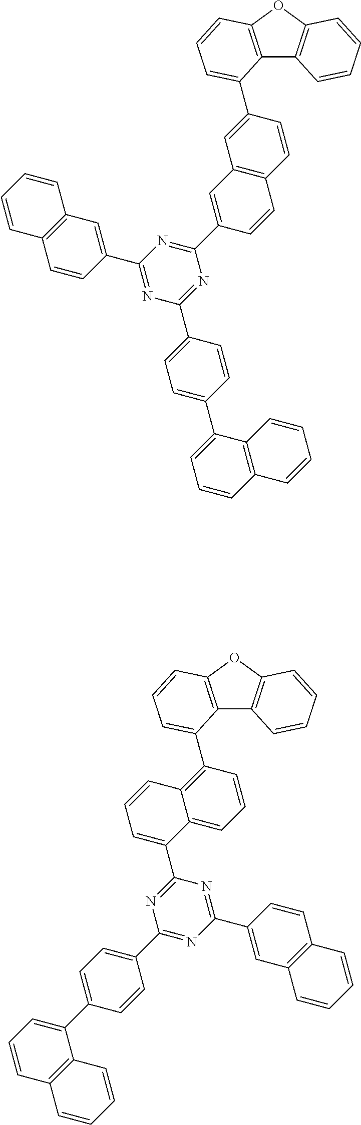 [SMILES: c1ccc2cc(-c3nc(-c4ccc(-c5cccc6ccccc56)cc4)nc(-c4ccc5ccc(-c6cccc7oc8ccccc8c67)cc5c4)n3)ccc2c1 ; c1ccc2cc(-c3nc(-c4ccc(-c5cccc6ccccc56)cc4)nc(-c4cccc5c(-c6cccc7oc8ccccc8c67)cccc45)n3)ccc2c1]

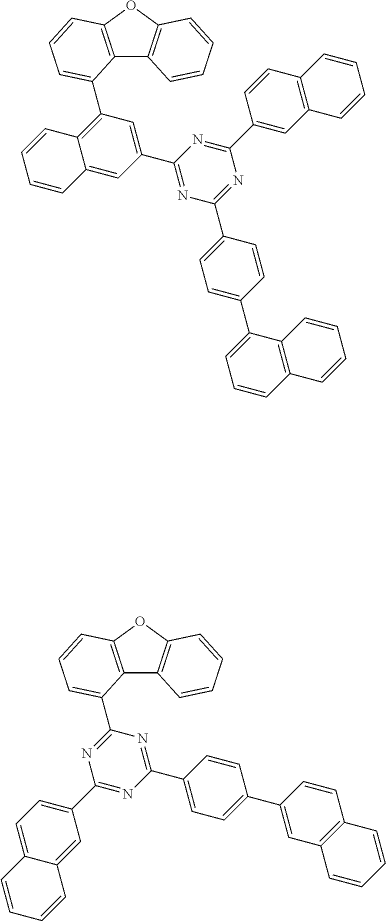 [SMILES: c1ccc2cc(-c3nc(-c4ccc(-c5cccc6ccccc56)cc4)nc(-c4cc(-c5cccc6oc7ccccc7c56)c5ccccc5c4)n3)ccc2c1 ; c1ccc2cc(-c3ccc(-c4nc(-c5ccc6ccccc6c5)nc(-c5cccc6oc7ccccc7c56)n4)cc3)ccc2c1]

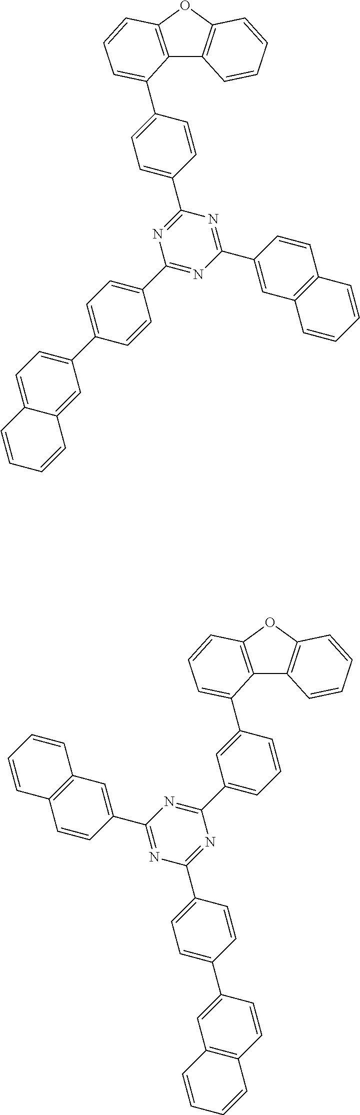 [SMILES: c1ccc2cc(-c3ccc(-c4nc(-c5ccc(-c6cccc7oc8ccccc8c67)cc5)nc(-c5ccc6ccccc6c5)n4)cc3)ccc2c1 ; c1cc(-c2nc(-c3ccc(-c4ccc5ccccc5c4)cc3)nc(-c3ccc4ccccc4c3)n2)cc(-c2cccc3oc4ccccc4c23)c1]

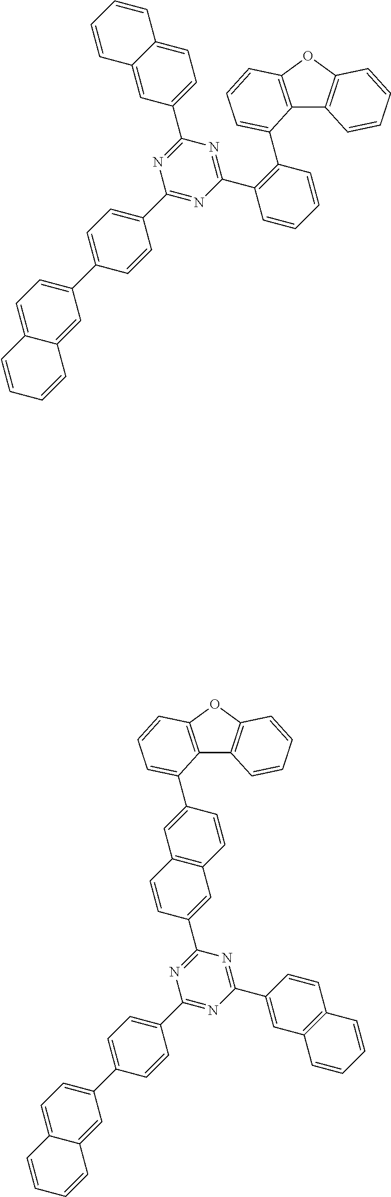 [SMILES: c1ccc(-c2cccc3oc4ccccc4c23)c(-c2nc(-c3ccc(-c4ccc5ccccc5c4)cc3)nc(-c3ccc4ccccc4c3)n2)c1 ; c1ccc2cc(-c3ccc(-c4nc(-c5ccc6ccccc6c5)nc(-c5ccc6cc(-c7cccc8oc9ccccc9c78)ccc6c5)n4)cc3)ccc2c1]

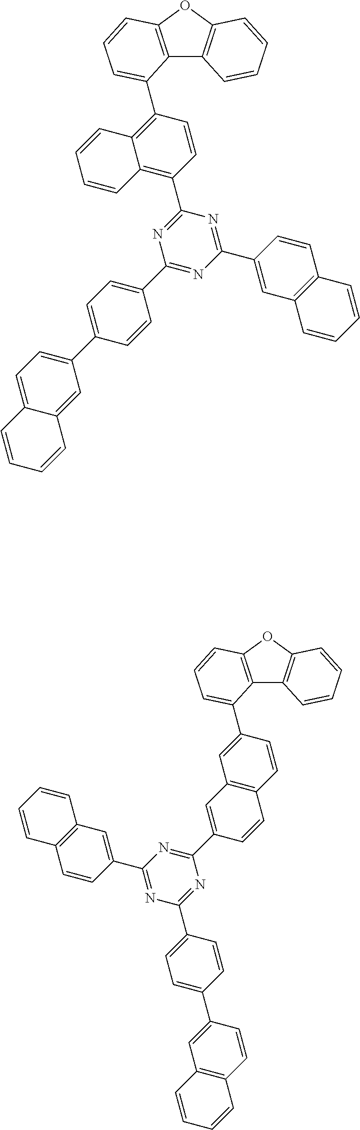 [SMILES: c1ccc2cc(-c3ccc(-c4nc(-c5ccc6ccccc6c5)nc(-c5ccc(-c6cccc7oc8ccccc8c67)c6ccccc56)n4)cc3)ccc2c1 ; c1ccc2cc(-c3ccc(-c4nc(-c5ccc6ccccc6c5)nc(-c5ccc6ccc(-c7cccc8oc9ccccc9c78)cc6c5)n4)cc3)ccc2c1]

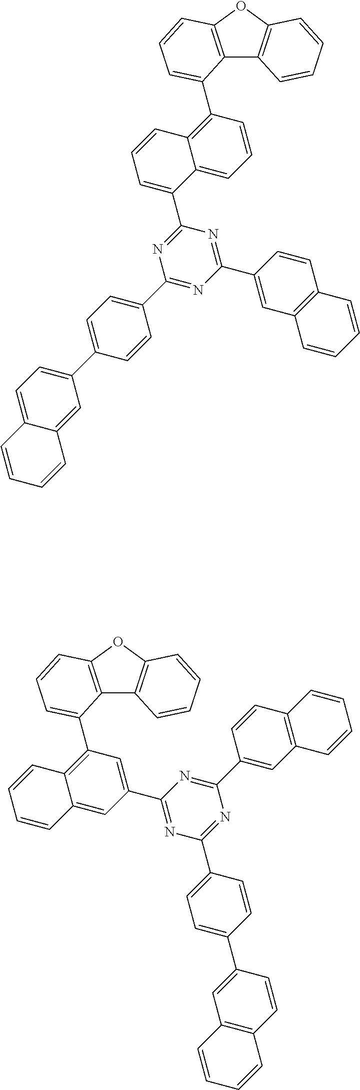 [SMILES: c1ccc2cc(-c3ccc(-c4nc(-c5ccc6ccccc6c5)nc(-c5cccc6c(-c7cccc8oc9ccccc9c78)cccc56)n4)cc3)ccc2c1 ; c1ccc2cc(-c3ccc(-c4nc(-c5ccc6ccccc6c5)nc(-c5cc(-c6cccc7oc8ccccc8c67)c6ccccc6c5)n4)cc3)ccc2c1]

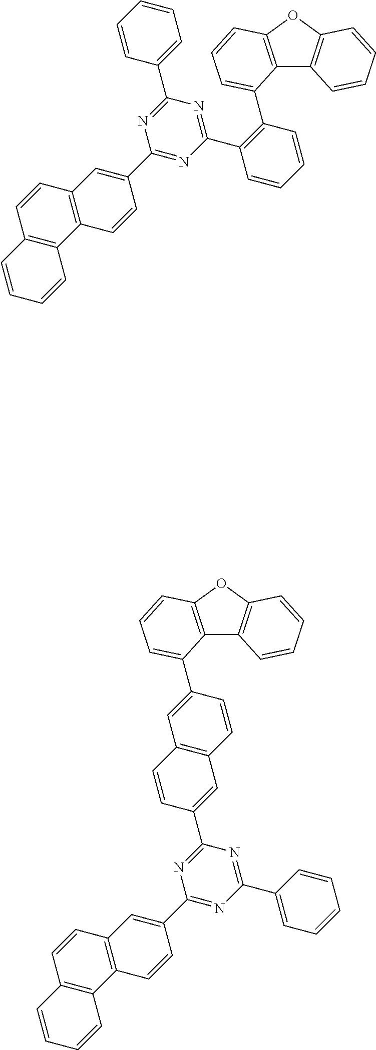 [SMILES: c1ccc(-c2nc(-c3ccc4c(ccc5ccccc54)c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(ccc5ccccc54)c3)n2)cc1]

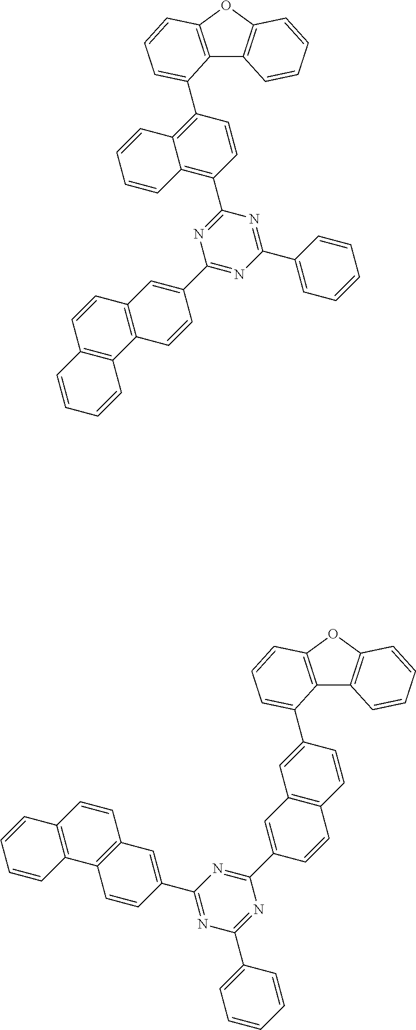 [SMILES: c1ccc(-c2nc(-c3ccc4c(ccc5ccccc54)c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(ccc5ccccc54)c3)n2)cc1]

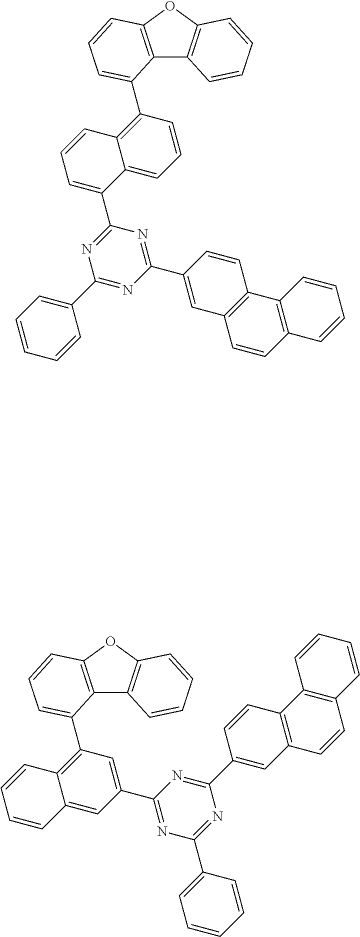 [SMILES: c1ccc(-c2nc(-c3ccc4c(ccc5ccccc54)c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(ccc5ccccc54)c3)n2)cc1]

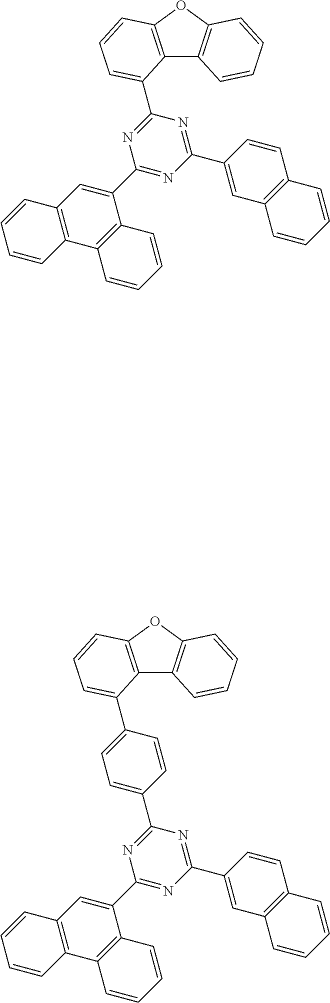 [SMILES: c1ccc2cc(-c3nc(-c4cc5ccccc5c5ccccc45)nc(-c4cccc5oc6ccccc6c45)n3)ccc2c1 ; c1ccc2cc(-c3nc(-c4ccc(-c5cccc6oc7ccccc7c56)cc4)nc(-c4cc5ccccc5c5ccccc45)n3)ccc2c1]

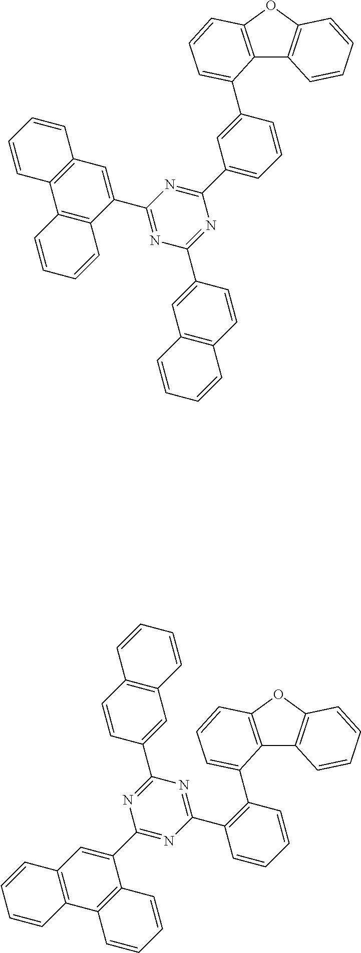 [SMILES: c1cc(-c2nc(-c3ccc4ccccc4c3)nc(-c3cc4ccccc4c4ccccc34)n2)cc(-c2cccc3oc4ccccc4c23)c1 ; c1ccc(-c2cccc3oc4ccccc4c23)c(-c2nc(-c3ccc4ccccc4c3)nc(-c3cc4ccccc4c4ccccc34)n2)c1]

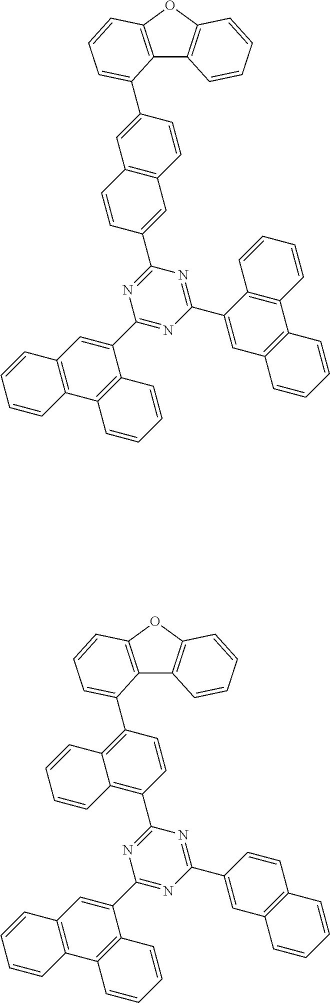 [SMILES: c1ccc2c(c1)cc(-c1nc(-c3ccc4cc(-c5cccc6oc7ccccc7c56)ccc4c3)nc(-c3cc4ccccc4c4ccccc34)n1)c1ccccc12 ; c1ccc2cc(-c3nc(-c4ccc(-c5cccc6oc7ccccc7c56)c5ccccc45)nc(-c4cc5ccccc5c5ccccc45)n3)ccc2c1]

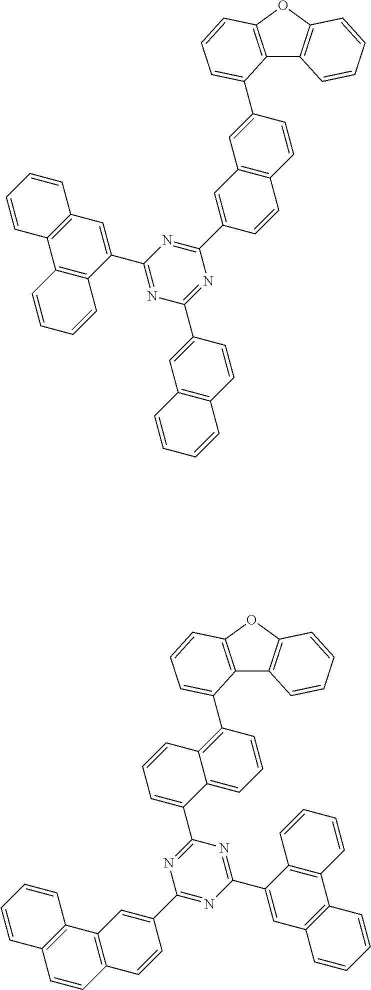 [SMILES: c1ccc2cc(-c3nc(-c4ccc5ccc(-c6cccc7oc8ccccc8c67)cc5c4)nc(-c4cc5ccccc5c5ccccc45)n3)ccc2c1 ; c1ccc2c(c1)ccc1ccc(-c3nc(-c4cccc5c(-c6cccc7oc8ccccc8c67)cccc45)nc(-c4cc5ccccc5c5ccccc45)n3)cc12]

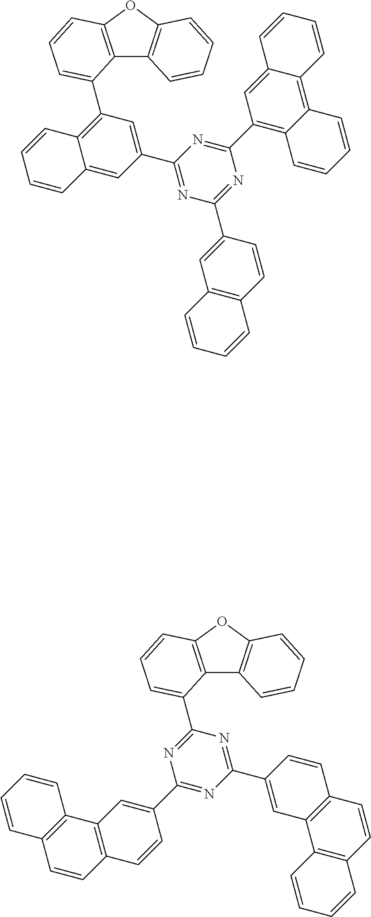 [SMILES: c1ccc2cc(-c3nc(-c4cc(-c5cccc6oc7ccccc7c56)c5ccccc5c4)nc(-c4cc5ccccc5c5ccccc45)n3)ccc2c1 ; c1ccc2c(c1)ccc1ccc(-c3nc(-c4ccc5ccc6ccccc6c5c4)nc(-c4cccc5oc6ccccc6c45)n3)cc12]

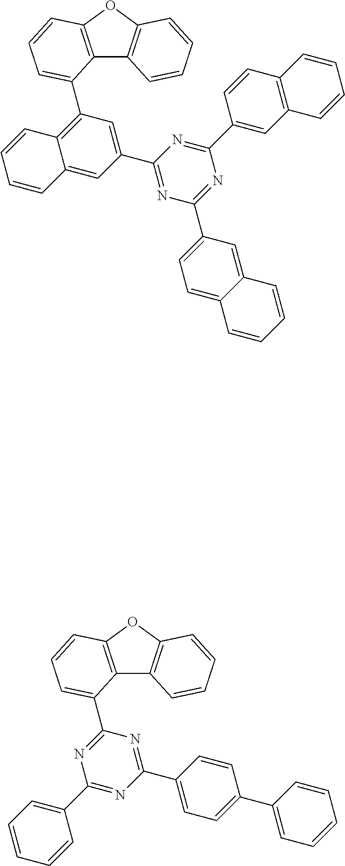 [SMILES: c1ccc2cc(-c3nc(-c4ccc5ccccc5c4)nc(-c4cc(-c5cccc6oc7ccccc7c56)c5ccccc5c4)n3)ccc2c1 ; c1ccc(-c2ccc(-c3nc(-c4ccccc4)nc(-c4cccc5oc6ccccc6c45)n3)cc2)cc1]

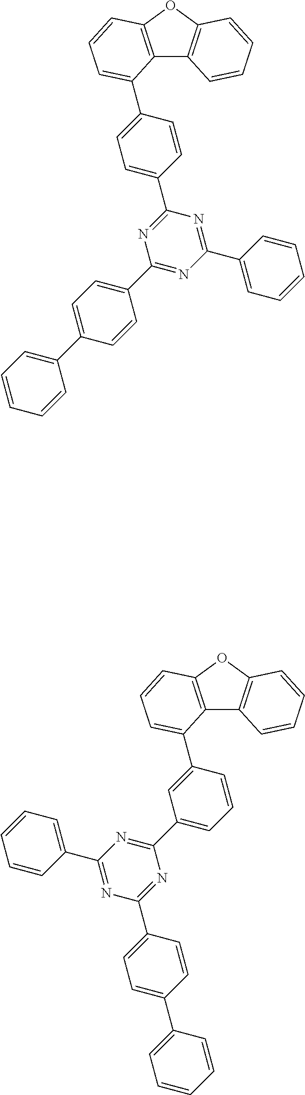 [SMILES: c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4cccc(-c5cccc6oc7ccccc7c56)c4)n3)cc2)cc1]

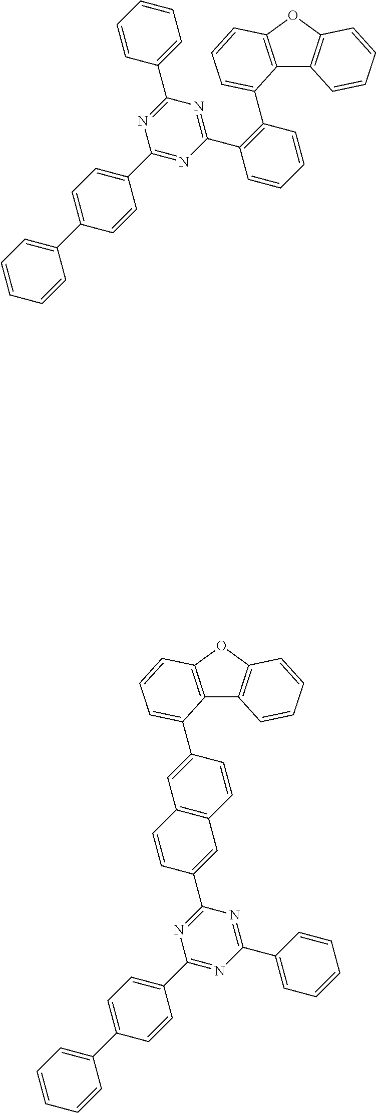 [SMILES: c1ccc(-c2ccc(-c3nc(-c4ccccc4)nc(-c4ccccc4-c4cccc5oc6ccccc6c45)n3)cc2)cc1 ; c1ccc(-c2ccc(-c3nc(-c4ccccc4)nc(-c4ccc5cc(-c6cccc7oc8ccccc8c67)ccc5c4)n3)cc2)cc1]

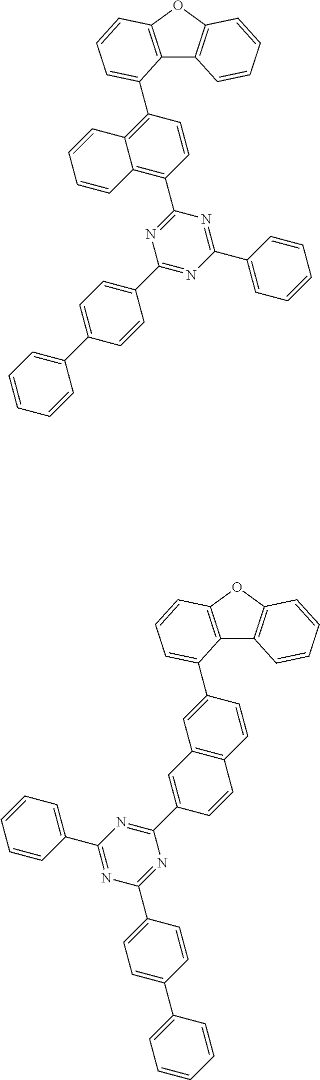 [SMILES: c1ccc(-c2ccc(-c3nc(-c4ccccc4)nc(-c4ccc(-c5cccc6oc7ccccc7c56)c5ccccc45)n3)cc2)cc1 ; c1ccc(-c2ccc(-c3nc(-c4ccccc4)nc(-c4ccc5ccc(-c6cccc7oc8ccccc8c67)cc5c4)n3)cc2)cc1]

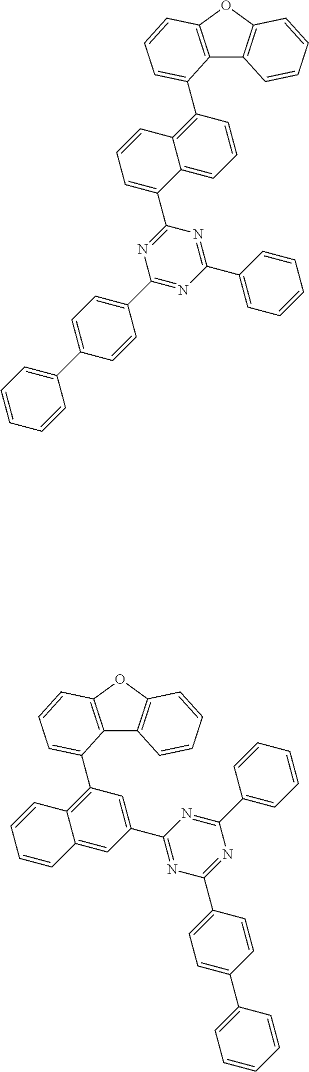 [SMILES: c1ccc(-c2ccc(-c3nc(-c4ccccc4)nc(-c4cccc5c(-c6cccc7oc8ccccc8c67)cccc45)n3)cc2)cc1 ; c1ccc(-c2ccc(-c3nc(-c4ccccc4)nc(-c4cc(-c5cccc6oc7ccccc7c56)c5ccccc5c4)n3)cc2)cc1]

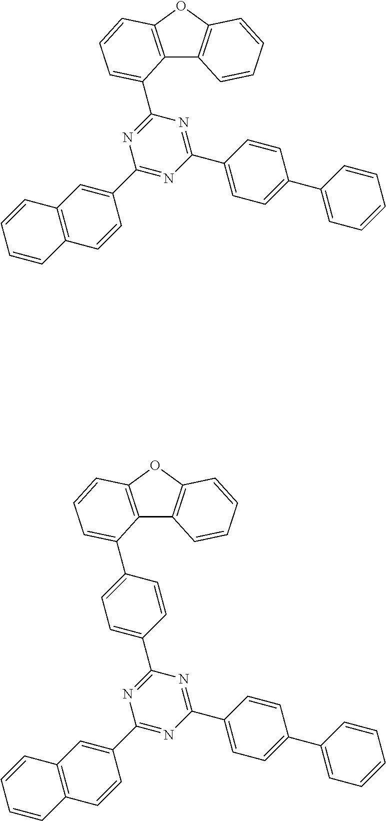 [SMILES: c1ccc(-c2ccc(-c3nc(-c4ccc5ccccc5c4)nc(-c4cccc5oc6ccccc6c45)n3)cc2)cc1 ; c1ccc(-c2ccc(-c3nc(-c4ccc(-c5cccc6oc7ccccc7c56)cc4)nc(-c4ccc5ccccc5c4)n3)cc2)cc1]

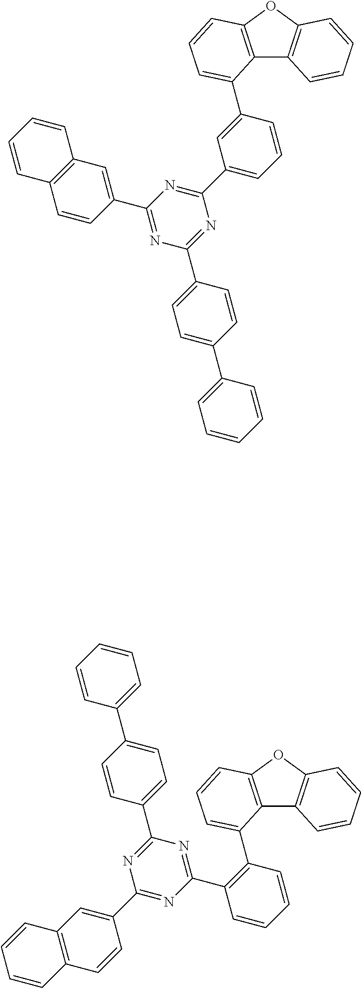 [SMILES: c1ccc(-c2ccc(-c3nc(-c4cccc(-c5cccc6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6ccccc6c45)n3)cc2)cc1]

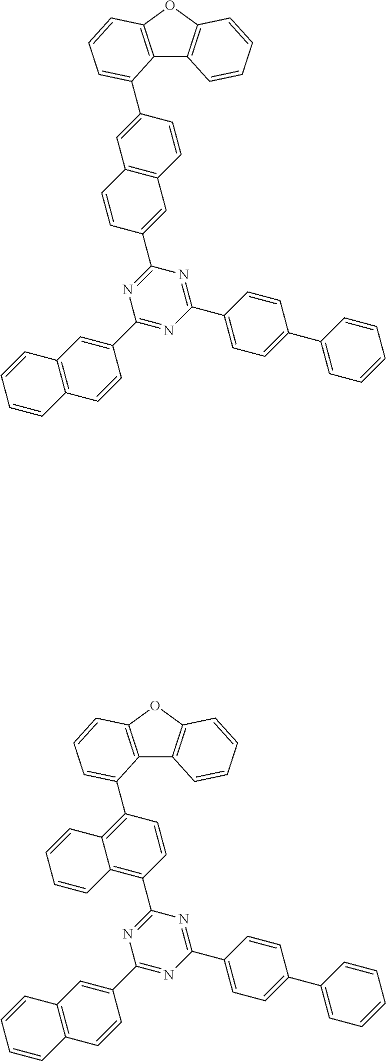 [SMILES: c1ccc(-c2ccc(-c3nc(-c4ccc5ccccc5c4)nc(-c4ccc5cc(-c6cccc7oc8ccccc8c67)ccc5c4)n3)cc2)cc1 ; c1ccc(-c2ccc(-c3nc(-c4ccc5ccccc5c4)nc(-c4ccc(-c5cccc6oc7ccccc7c56)c5ccccc45)n3)cc2)cc1]

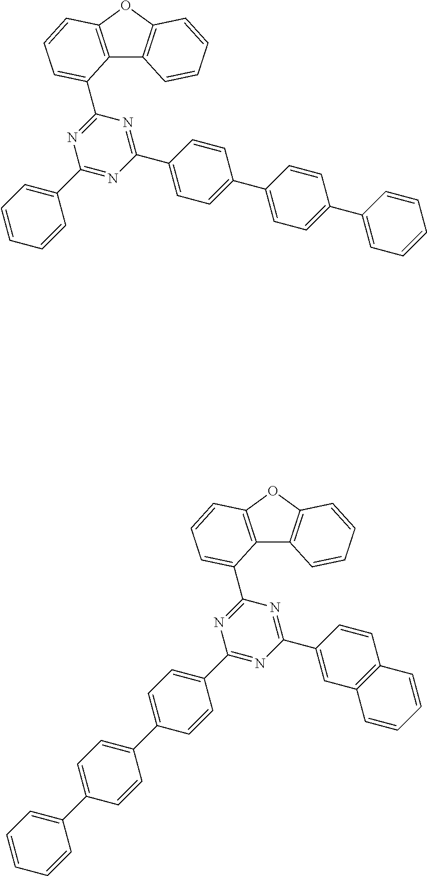 [SMILES: c1ccc(-c2ccc(-c3ccc(-c4nc(-c5ccccc5)nc(-c5cccc6oc7ccccc7c56)n4)cc3)cc2)cc1 ; c1ccc(-c2ccc(-c3ccc(-c4nc(-c5ccc6ccccc6c5)nc(-c5cccc6oc7ccccc7c56)n4)cc3)cc2)cc1]

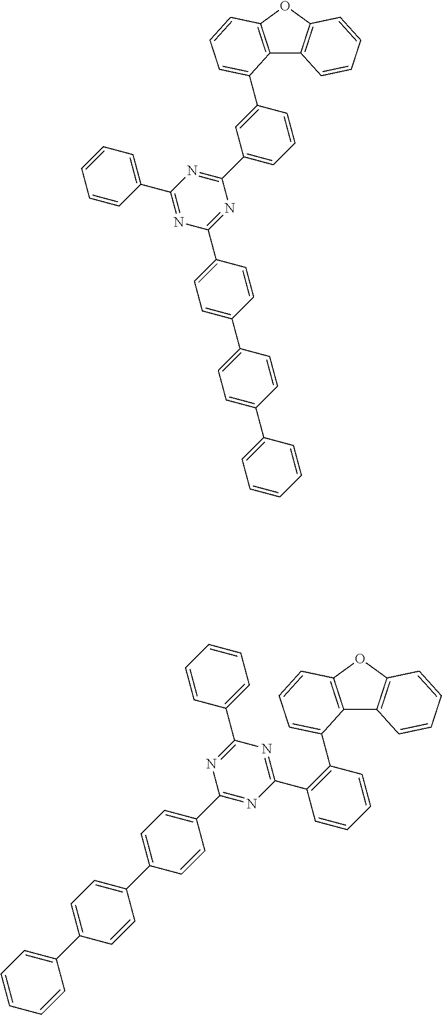 [SMILES: c1ccc(-c2ccc(-c3ccc(-c4nc(-c5ccccc5)nc(-c5cccc(-c6cccc7oc8ccccc8c67)c5)n4)cc3)cc2)cc1 ; c1ccc(-c2ccc(-c3ccc(-c4nc(-c5ccccc5)nc(-c5ccccc5-c5cccc6oc7ccccc7c56)n4)cc3)cc2)cc1]

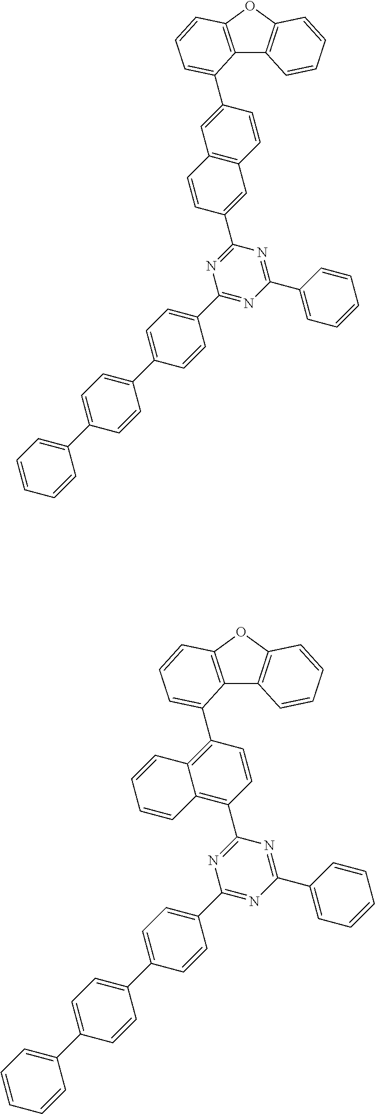 [SMILES: c1ccc(-c2ccc(-c3ccc(-c4nc(-c5ccccc5)nc(-c5ccc6cc(-c7cccc8oc9ccccc9c78)ccc6c5)n4)cc3)cc2)cc1 ; c1ccc(-c2ccc(-c3ccc(-c4nc(-c5ccccc5)nc(-c5ccc(-c6cccc7oc8ccccc8c67)c6ccccc56)n4)cc3)cc2)cc1]

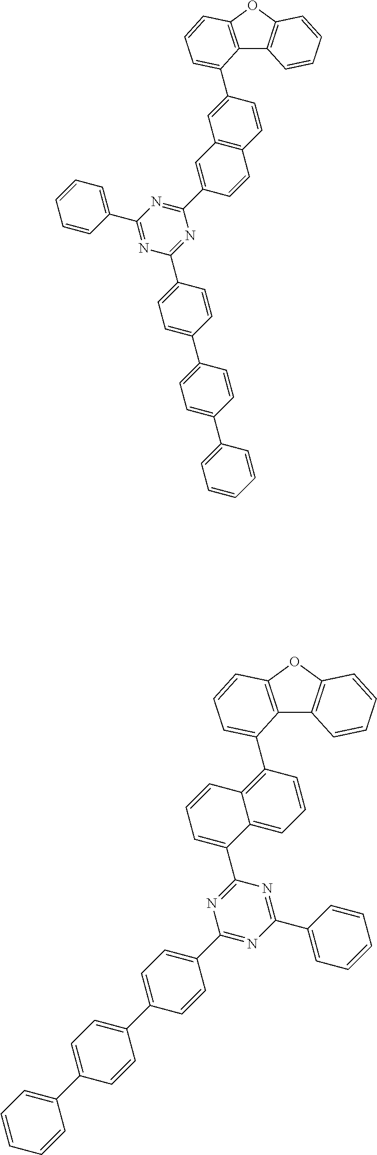 [SMILES: c1ccc(-c2ccc(-c3ccc(-c4nc(-c5ccccc5)nc(-c5ccc6ccc(-c7cccc8oc9ccccc9c78)cc6c5)n4)cc3)cc2)cc1 ; c1ccc(-c2ccc(-c3ccc(-c4nc(-c5ccccc5)nc(-c5cccc6c(-c7cccc8oc9ccccc9c78)cccc56)n4)cc3)cc2)cc1]

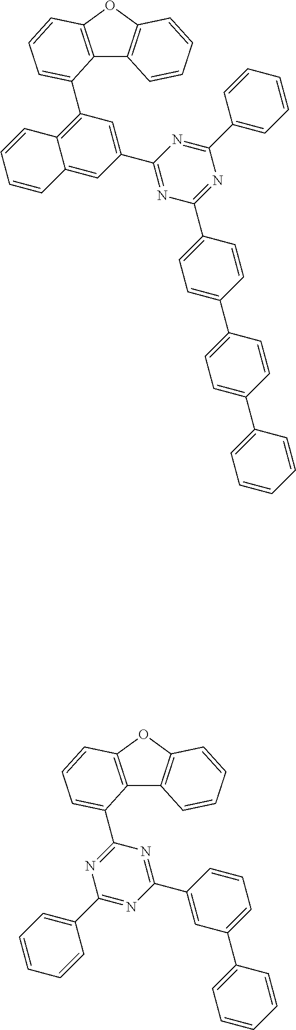 [SMILES: c1ccc(-c2ccc(-c3ccc(-c4nc(-c5ccccc5)nc(-c5cc(-c6cccc7oc8ccccc8c67)c6ccccc6c5)n4)cc3)cc2)cc1 ; c1ccc(-c2cccc(-c3nc(-c4ccccc4)nc(-c4cccc5oc6ccccc6c45)n3)c2)cc1]

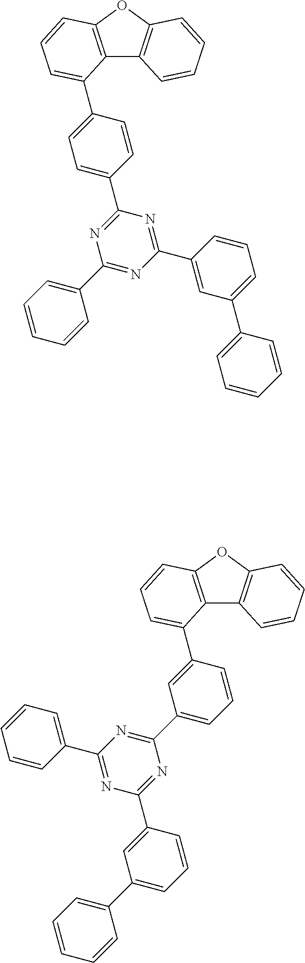 [SMILES: c1ccc(-c2cccc(-c3nc(-c4ccccc4)nc(-c4ccc(-c5cccc6oc7ccccc7c56)cc4)n3)c2)cc1 ; c1ccc(-c2cccc(-c3nc(-c4ccccc4)nc(-c4cccc(-c5cccc6oc7ccccc7c56)c4)n3)c2)cc1]

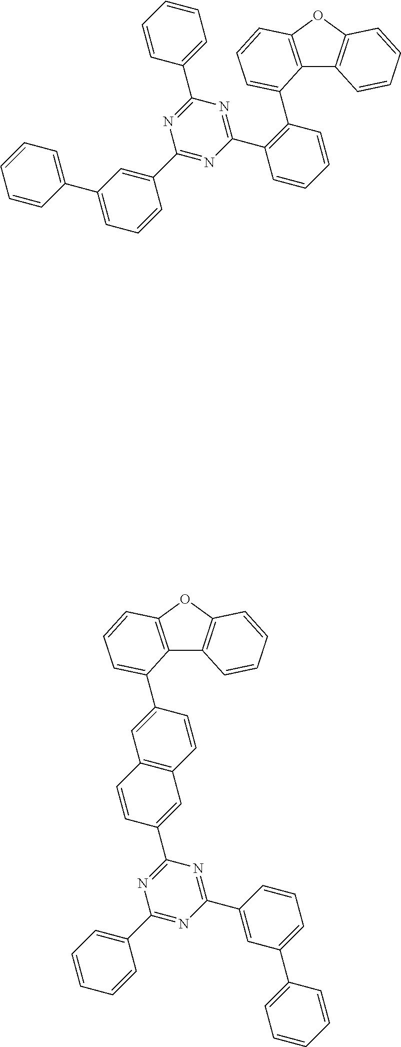 [SMILES: c1ccc(-c2cccc(-c3nc(-c4ccccc4)nc(-c4ccccc4-c4cccc5oc6ccccc6c45)n3)c2)cc1 ; c1ccc(-c2cccc(-c3nc(-c4ccccc4)nc(-c4ccc5cc(-c6cccc7oc8ccccc8c67)ccc5c4)n3)c2)cc1]

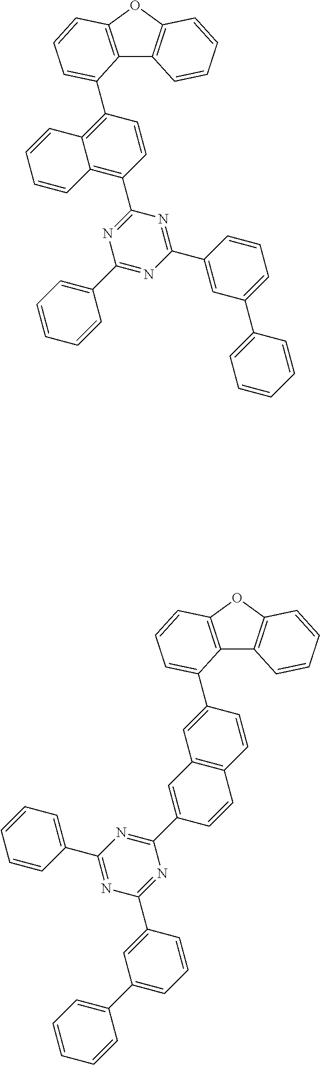 [SMILES: c1ccc(-c2cccc(-c3nc(-c4ccccc4)nc(-c4ccc(-c5cccc6oc7ccccc7c56)c5ccccc45)n3)c2)cc1 ; c1ccc(-c2cccc(-c3nc(-c4ccccc4)nc(-c4ccc5ccc(-c6cccc7oc8ccccc8c67)cc5c4)n3)c2)cc1]

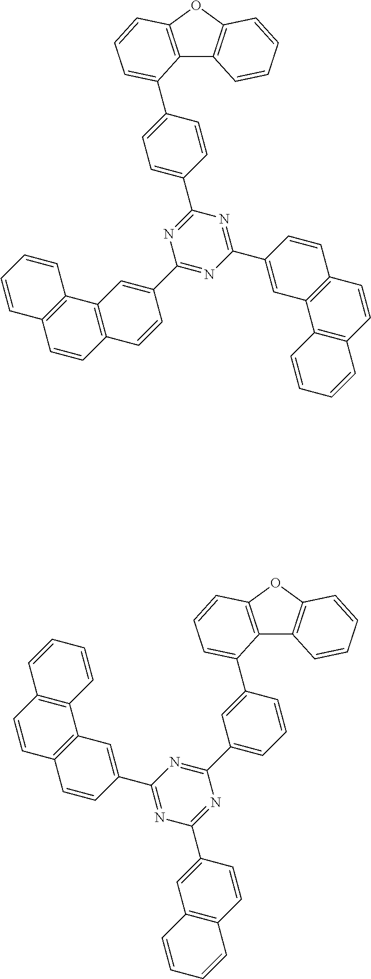 [SMILES: c1ccc2c(c1)ccc1ccc(-c3nc(-c4ccc(-c5cccc6oc7ccccc7c56)cc4)nc(-c4ccc5ccc6ccccc6c5c4)n3)cc12 ; c1cc(-c2nc(-c3ccc4ccccc4c3)nc(-c3ccc4ccc5ccccc5c4c3)n2)cc(-c2cccc3oc4ccccc4c23)c1]

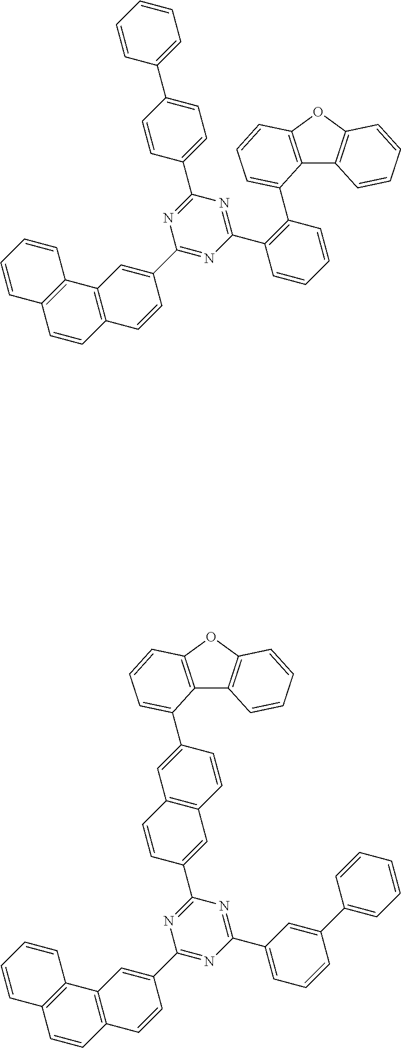 [SMILES: c1ccc(-c2ccc(-c3nc(-c4ccc5ccc6ccccc6c5c4)nc(-c4ccccc4-c4cccc5oc6ccccc6c45)n3)cc2)cc1 ; c1ccc(-c2cccc(-c3nc(-c4ccc5cc(-c6cccc7oc8ccccc8c67)ccc5c4)nc(-c4ccc5ccc6ccccc6c5c4)n3)c2)cc1]

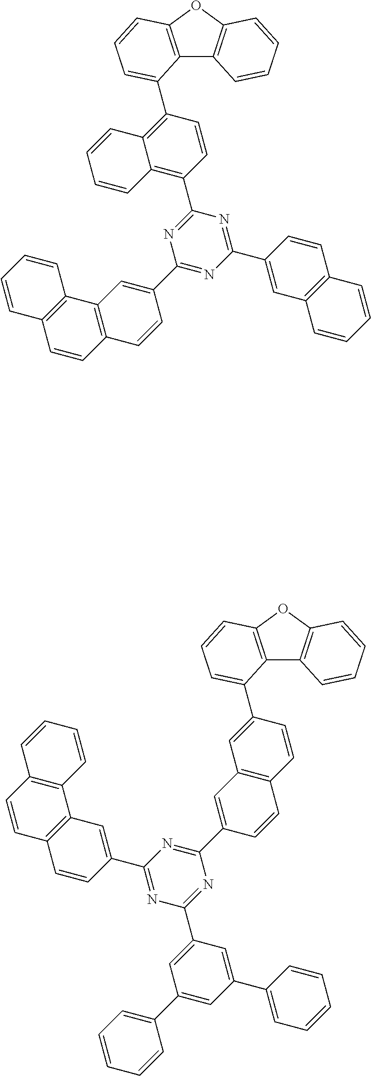 [SMILES: c1ccc2cc(-c3nc(-c4ccc5ccc6ccccc6c5c4)nc(-c4ccc(-c5cccc6oc7ccccc7c56)c5ccccc45)n3)ccc2c1 ; c1ccc(-c2cc(-c3ccccc3)cc(-c3nc(-c4ccc5ccc(-c6cccc7oc8ccccc8c67)cc5c4)nc(-c4ccc5ccc6ccccc6c5c4)n3)c2)cc1]

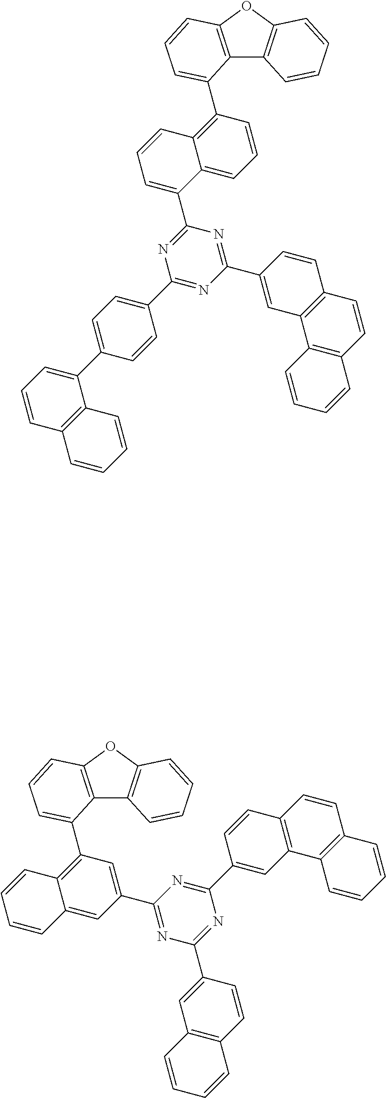 [SMILES: c1ccc2c(-c3ccc(-c4nc(-c5ccc6ccc7ccccc7c6c5)nc(-c5cccc6c(-c7cccc8oc9ccccc9c78)cccc56)n4)cc3)cccc2c1 ; c1ccc2cc(-c3nc(-c4cc(-c5cccc6oc7ccccc7c56)c5ccccc5c4)nc(-c4ccc5ccc6ccccc6c5c4)n3)ccc2c1]

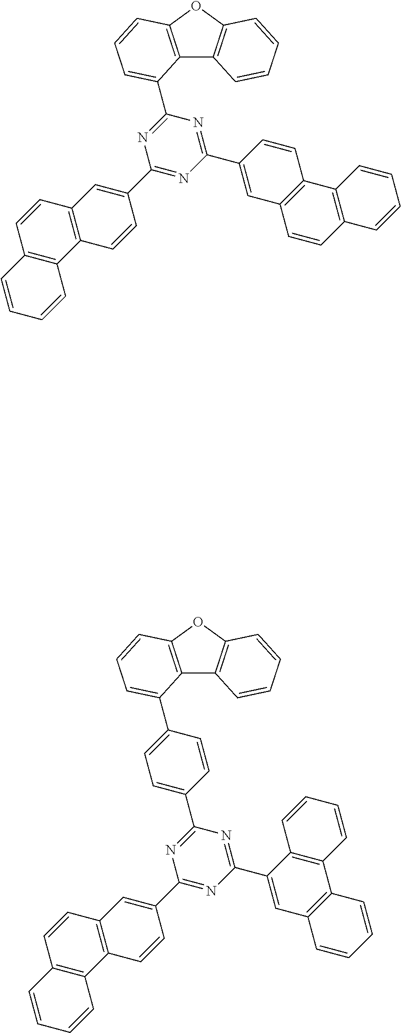 [SMILES: c1ccc2c(c1)ccc1cc(-c3nc(-c4ccc5c(ccc6ccccc65)c4)nc(-c4cccc5oc6ccccc6c45)n3)ccc12 ; c1ccc2c(c1)ccc1cc(-c3nc(-c4ccc(-c5cccc6oc7ccccc7c56)cc4)nc(-c4cc5ccccc5c5ccccc45)n3)ccc12]

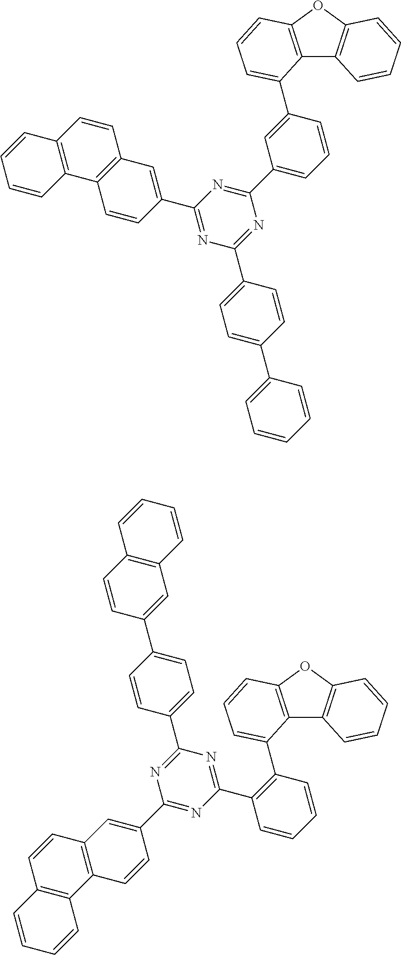 [SMILES: c1ccc(-c2ccc(-c3nc(-c4cccc(-c5cccc6oc7ccccc7c56)c4)nc(-c4ccc5c(ccc6ccccc65)c4)n3)cc2)cc1 ; c1ccc(-c2cccc3oc4ccccc4c23)c(-c2nc(-c3ccc(-c4ccc5ccccc5c4)cc3)nc(-c3ccc4c(ccc5ccccc54)c3)n2)c1]

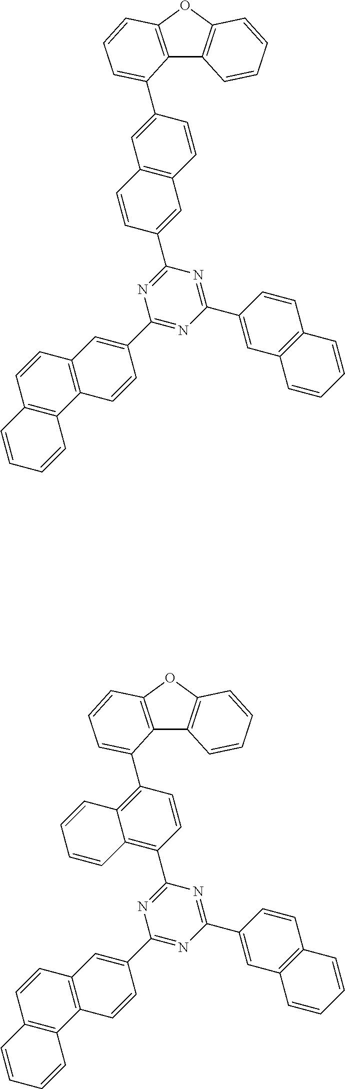 [SMILES: c1ccc2cc(-c3nc(-c4ccc5cc(-c6cccc7oc8ccccc8c67)ccc5c4)nc(-c4ccc5c(ccc6ccccc65)c4)n3)ccc2c1 ; c1ccc2cc(-c3nc(-c4ccc5c(ccc6ccccc65)c4)nc(-c4ccc(-c5cccc6oc7ccccc7c56)c5ccccc45)n3)ccc2c1]

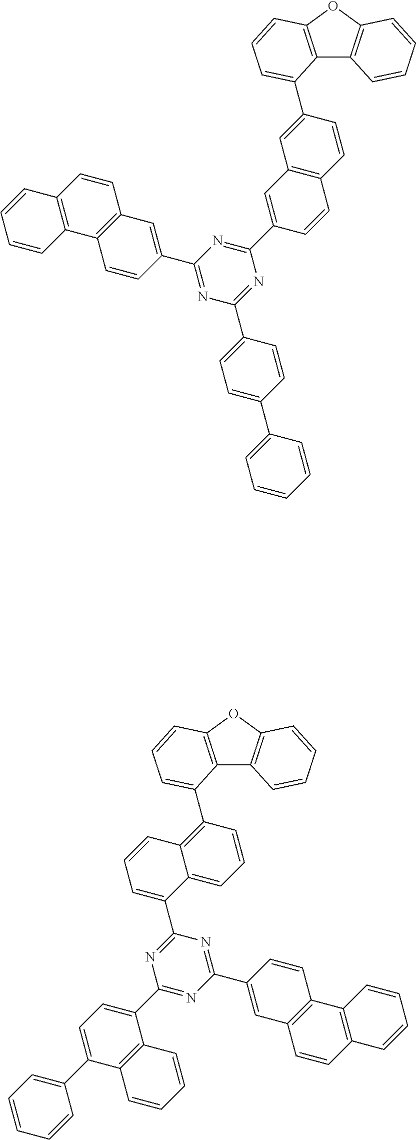 [SMILES: c1ccc(-c2ccc(-c3nc(-c4ccc5ccc(-c6cccc7oc8ccccc8c67)cc5c4)nc(-c4ccc5c(ccc6ccccc65)c4)n3)cc2)cc1 ; c1ccc(-c2ccc(-c3nc(-c4ccc5c(ccc6ccccc65)c4)nc(-c4cccc5c(-c6cccc7oc8ccccc8c67)cccc45)n3)c3ccccc23)cc1]

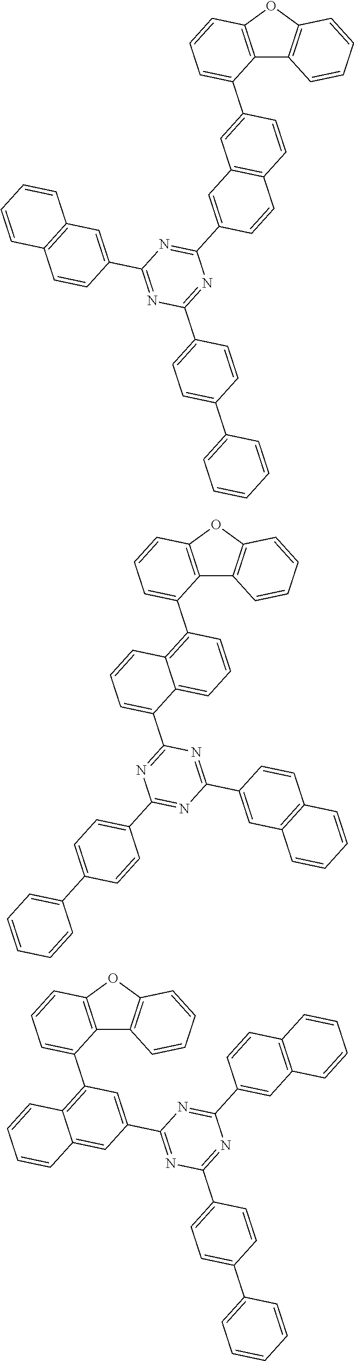

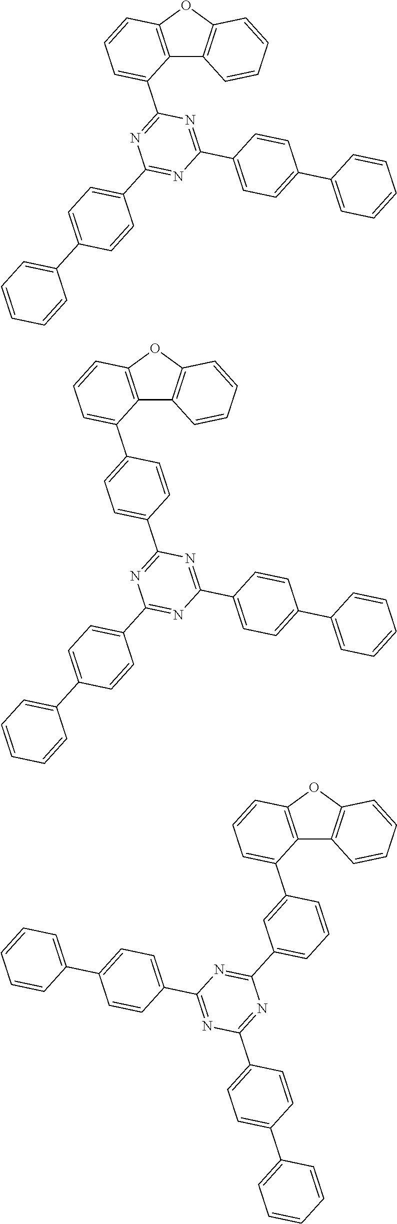 [SMILES: c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4ccc(-c5ccccc5)cc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1]

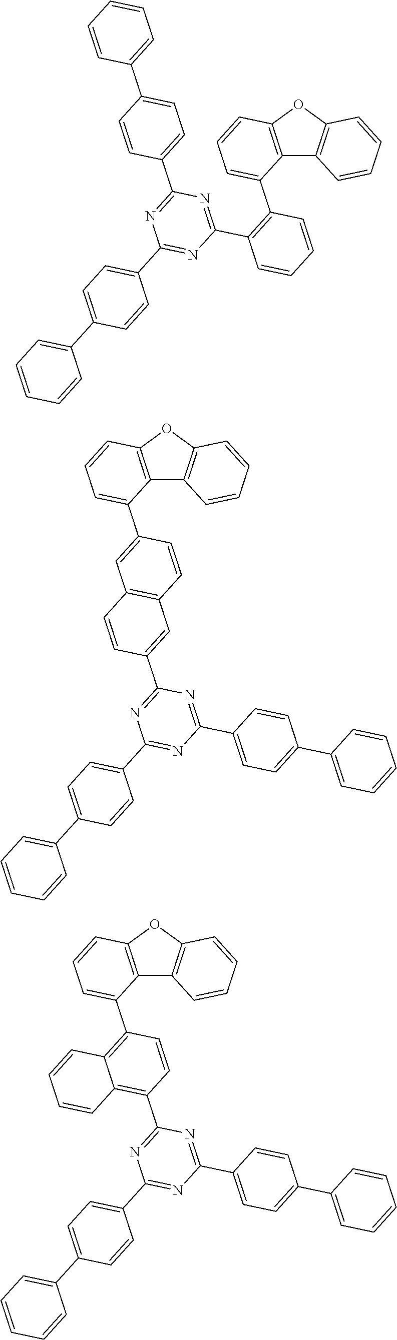 [SMILES: c1ccc(-c2ccc(-c3nc(-c4ccc(-c5ccccc5)cc4)nc(-c4ccccc4-c4cccc5oc6ccccc6c45)n3)cc2)cc1 ; c1ccc(-c2ccc(-c3nc(-c4ccc(-c5ccccc5)cc4)nc(-c4ccc5cc(-c6cccc7oc8ccccc8c67)ccc5c4)n3)cc2)cc1 ; c1ccc(-c2ccc(-c3nc(-c4ccc(-c5ccccc5)cc4)nc(-c4ccc(-c5cccc6oc7ccccc7c56)c5ccccc45)n3)cc2)cc1]

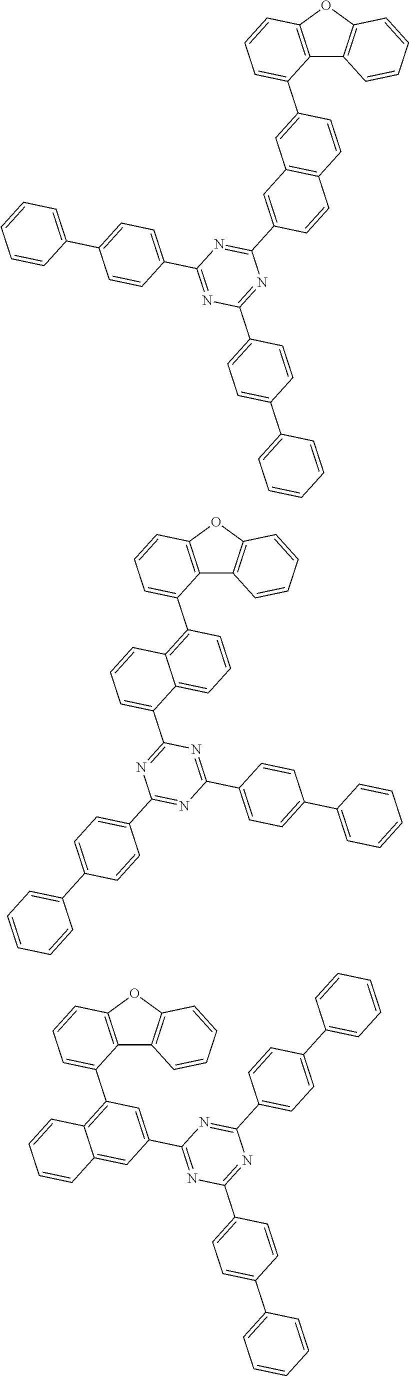

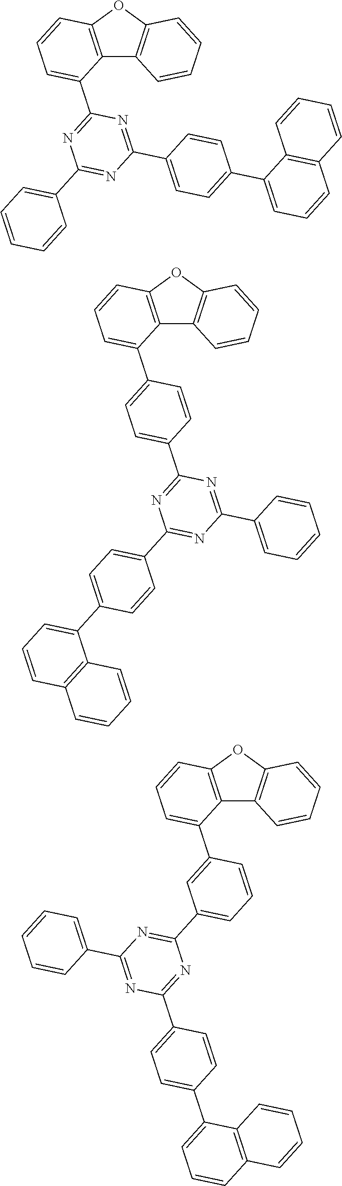 [SMILES: c1ccc(-c2nc(-c3ccc(-c4cccc5ccccc45)cc3)nc(-c3cccc4oc5ccccc5c34)n2)cc1 ; c1ccc(-c2nc(-c3ccc(-c4cccc5ccccc45)cc3)nc(-c3ccc(-c4cccc5oc6ccccc6c45)cc3)n2)cc1 ; c1ccc(-c2nc(-c3ccc(-c4cccc5ccccc45)cc3)nc(-c3cccc(-c4cccc5oc6ccccc6c45)c3)n2)cc1]

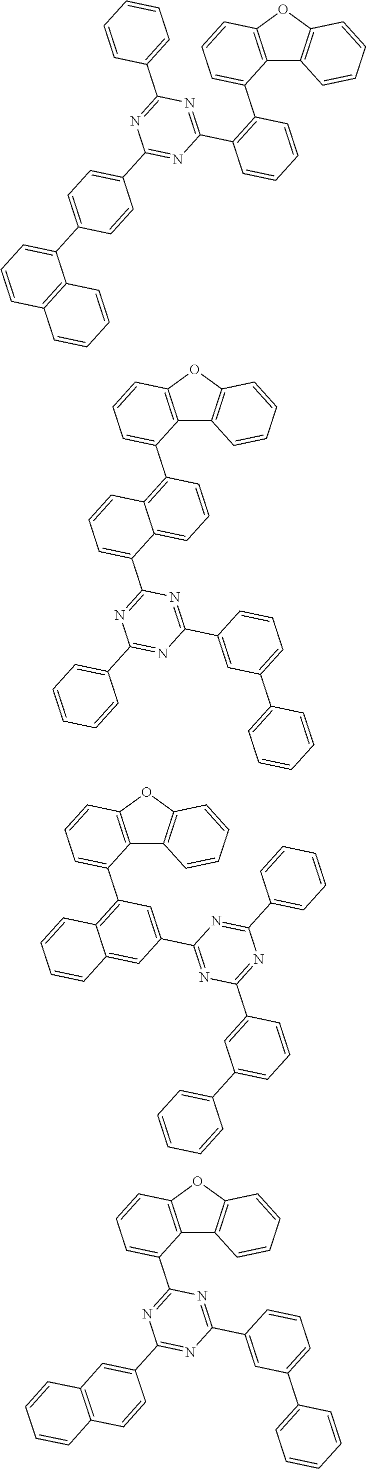

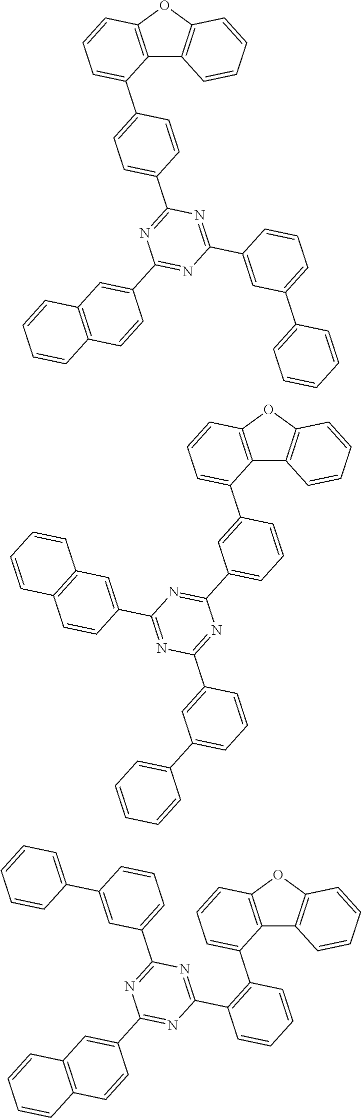

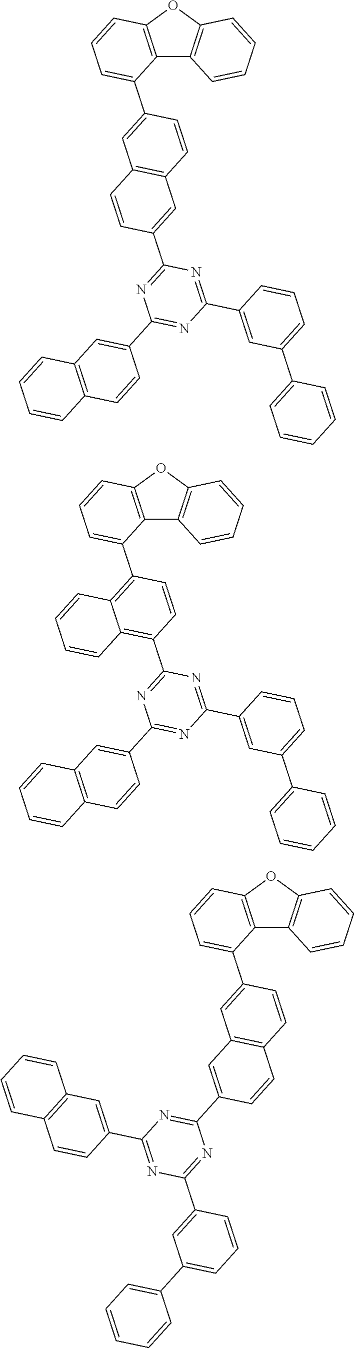

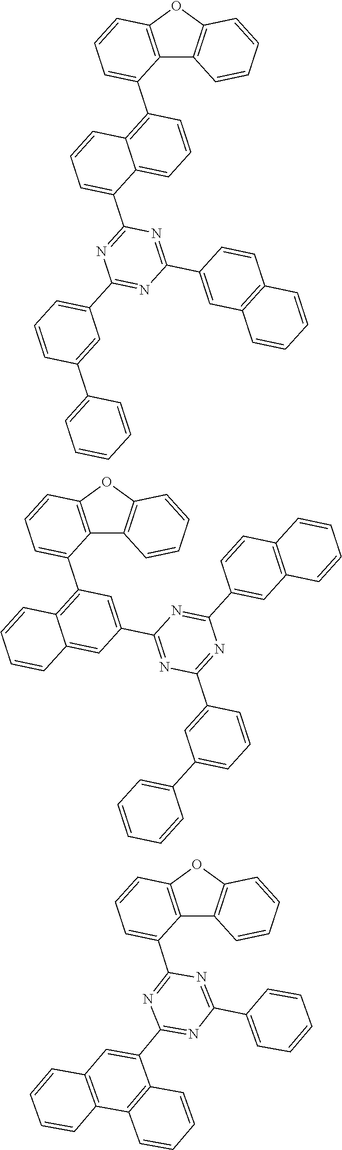

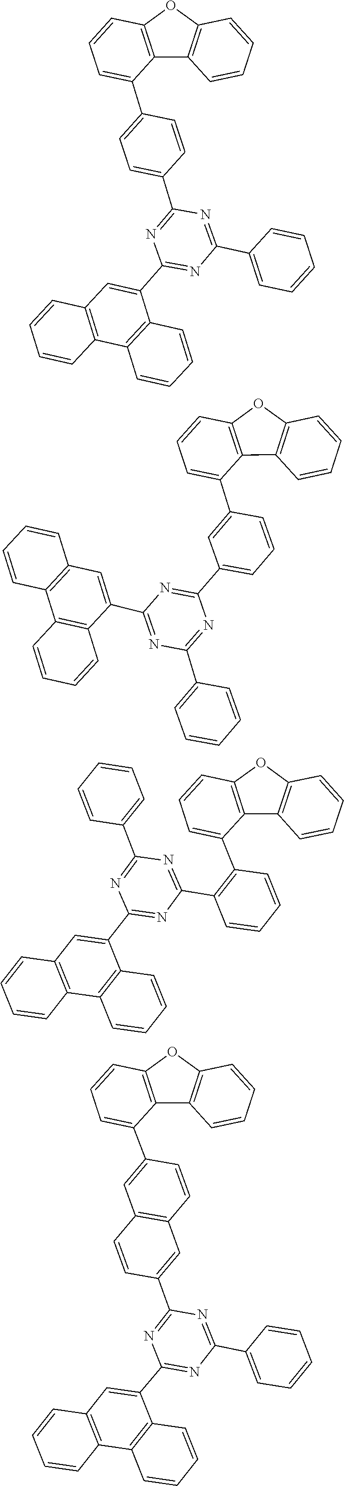

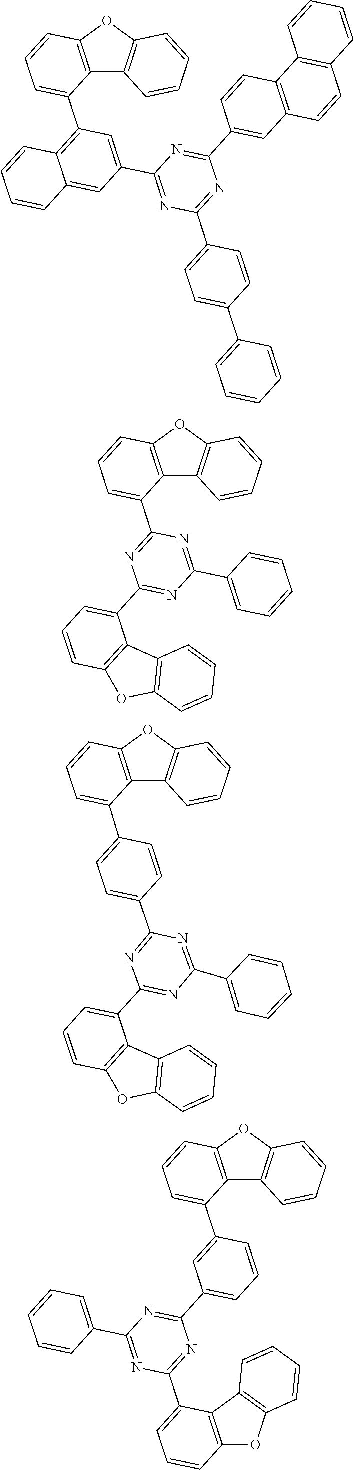

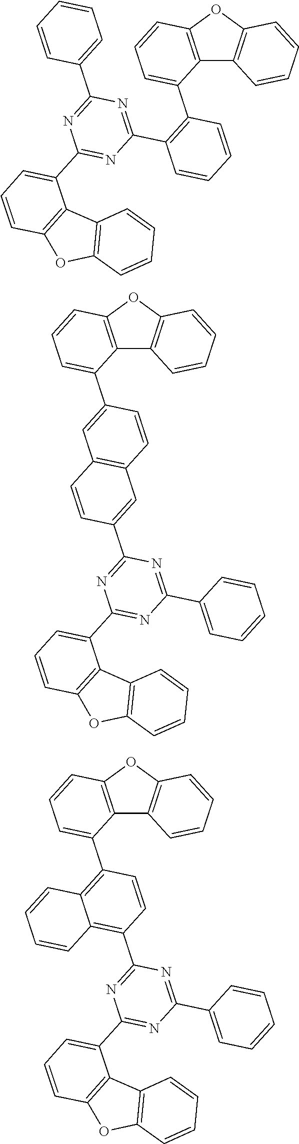

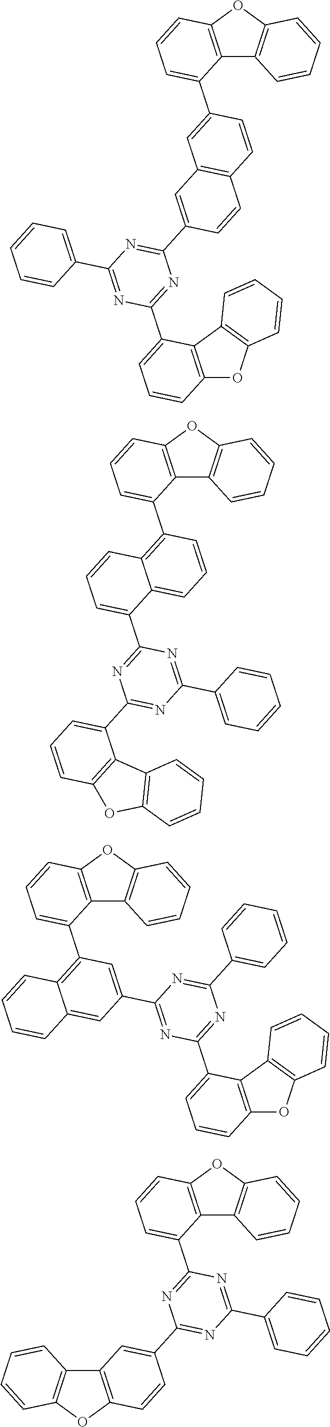

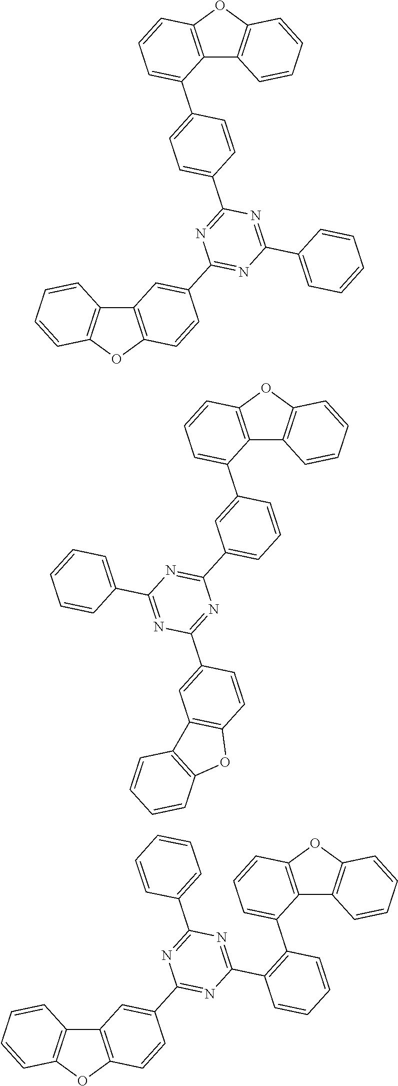

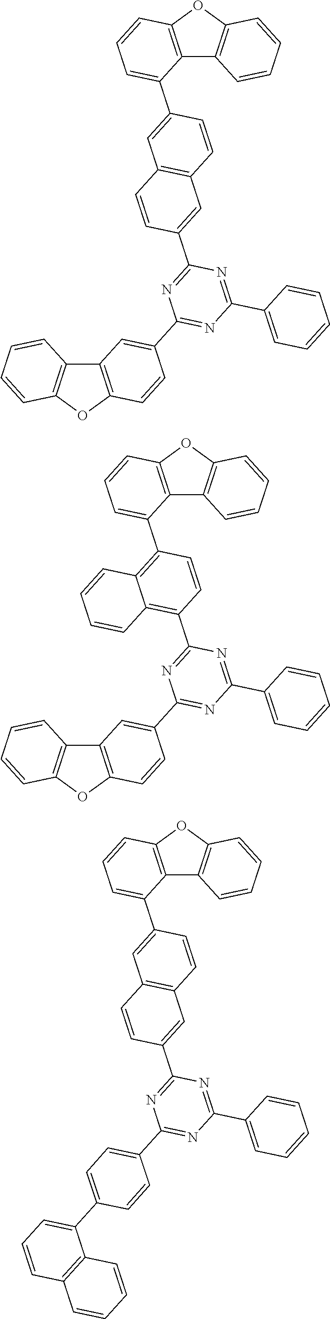 [SMILES: c1ccc(-c2nc(-c3ccc4cc(-c5cccc6oc7ccccc7c56)ccc4c3)nc(-c3ccc4oc5ccccc5c4c3)n2)cc1 ; c1ccc(-c2nc(-c3ccc4oc5ccccc5c4c3)nc(-c3ccc(-c4cccc5oc6ccccc6c45)c4ccccc34)n2)cc1 ; c1ccc(-c2nc(-c3ccc(-c4cccc5ccccc45)cc3)nc(-c3ccc4cc(-c5cccc6oc7ccccc7c56)ccc4c3)n2)cc1]

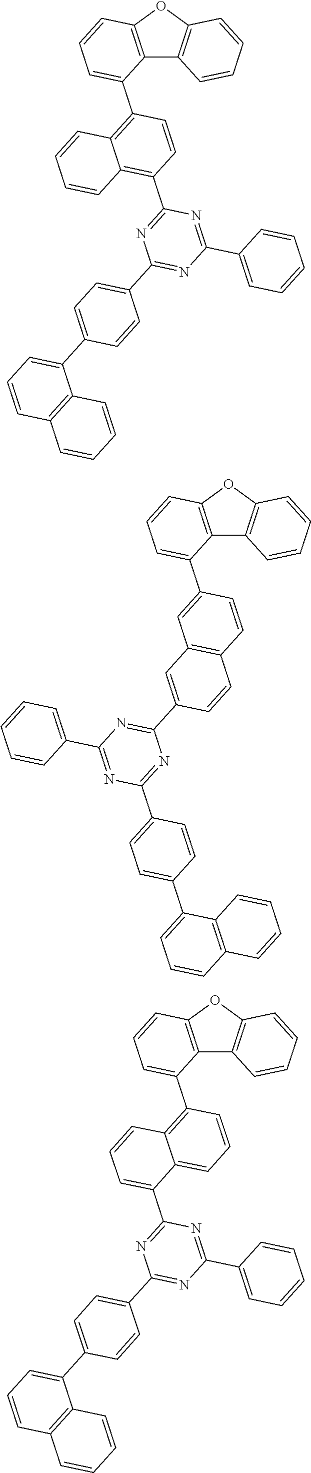

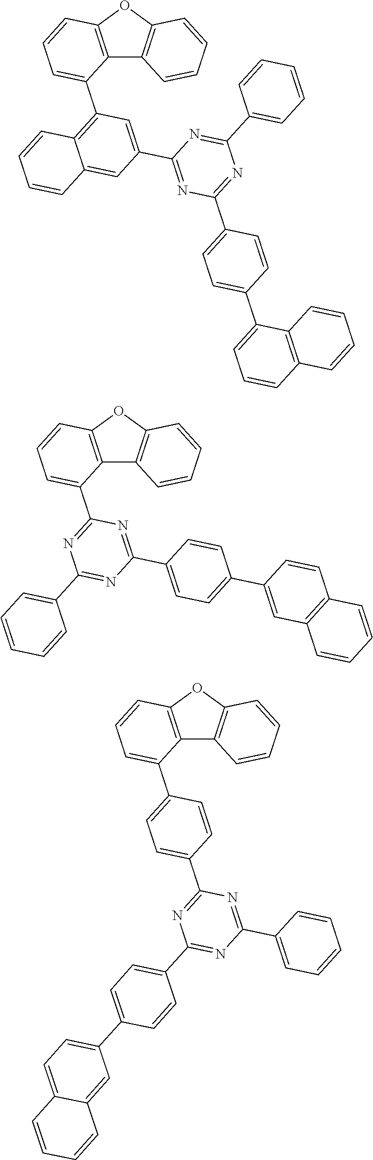 [SMILES: c1ccc(-c2nc(-c3ccc(-c4cccc5ccccc45)cc3)nc(-c3cc(-c4cccc5oc6ccccc6c45)c4ccccc4c3)n2)cc1 ; c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc(-c4cccc5oc6ccccc6c45)cc3)n2)cc1]

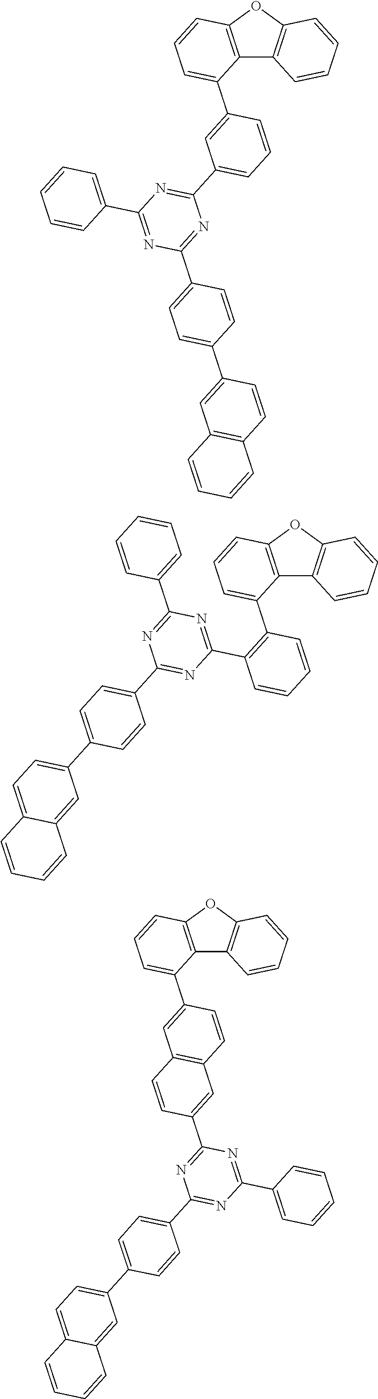

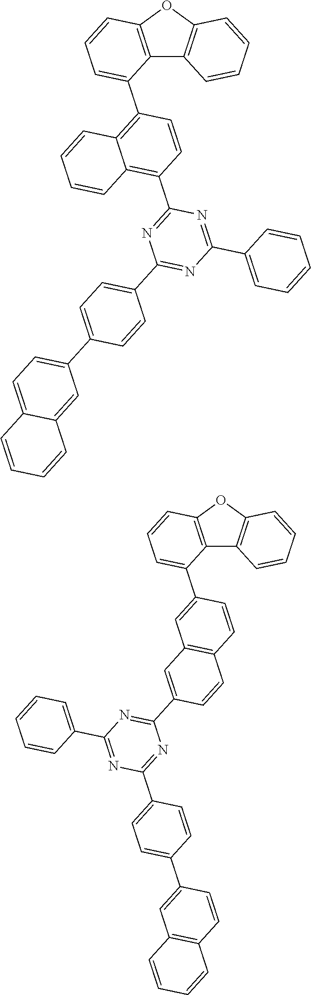 [SMILES: c1ccc(-c2nc(-c3ccc(-c4ccc5ccccc5c4)cc3)nc(-c3ccc(-c4cccc5oc6ccccc6c45)c4ccccc34)n2)cc1 ; c1ccc(-c2nc(-c3ccc(-c4ccc5ccccc5c4)cc3)nc(-c3ccc4ccc(-c5cccc6oc7ccccc7c56)cc4c3)n2)cc1]

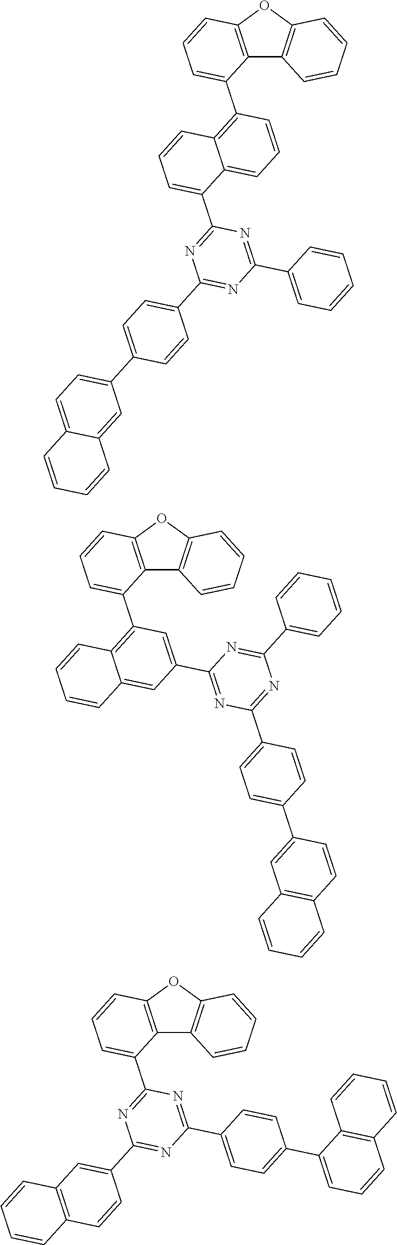

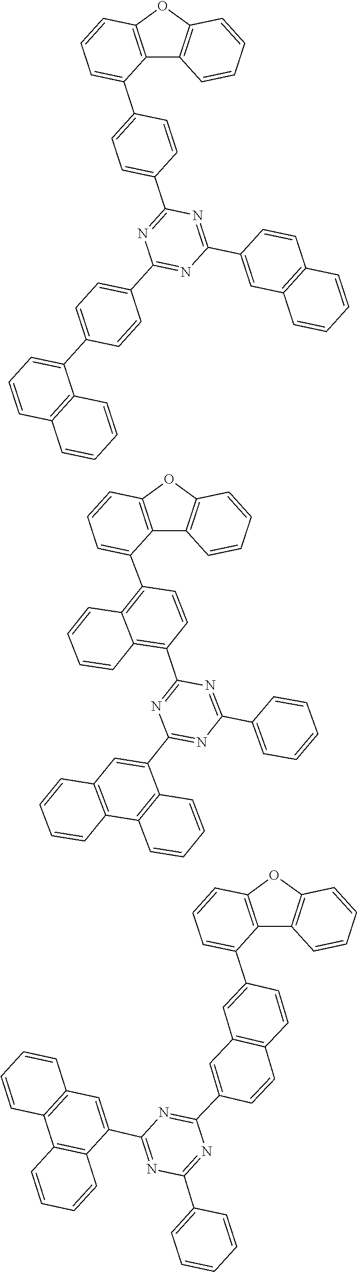

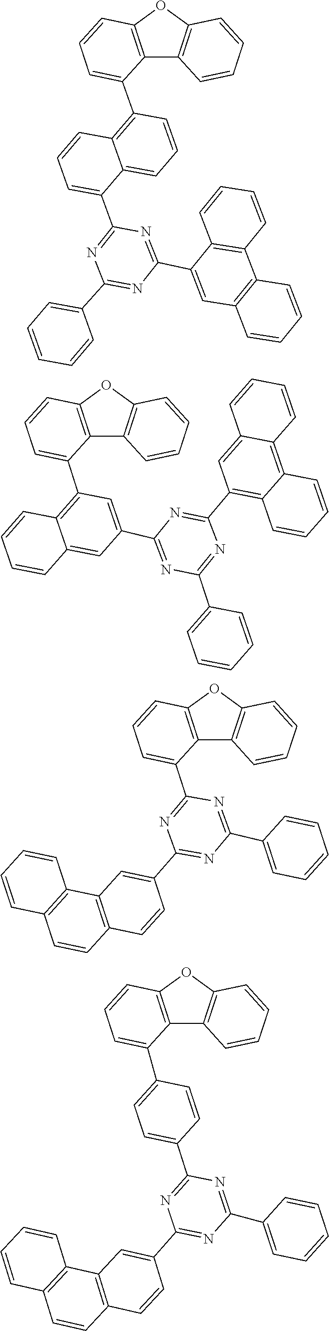

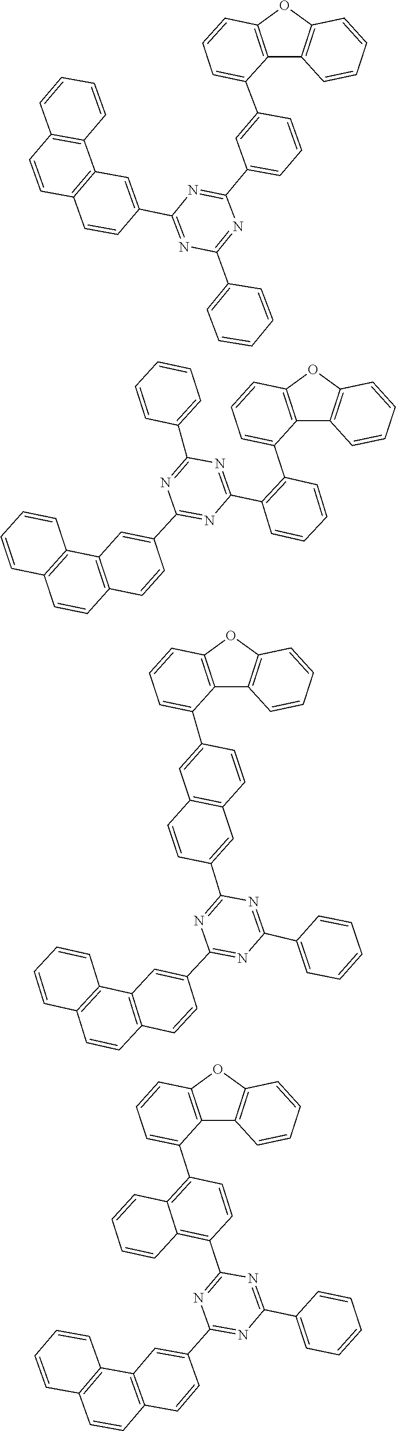

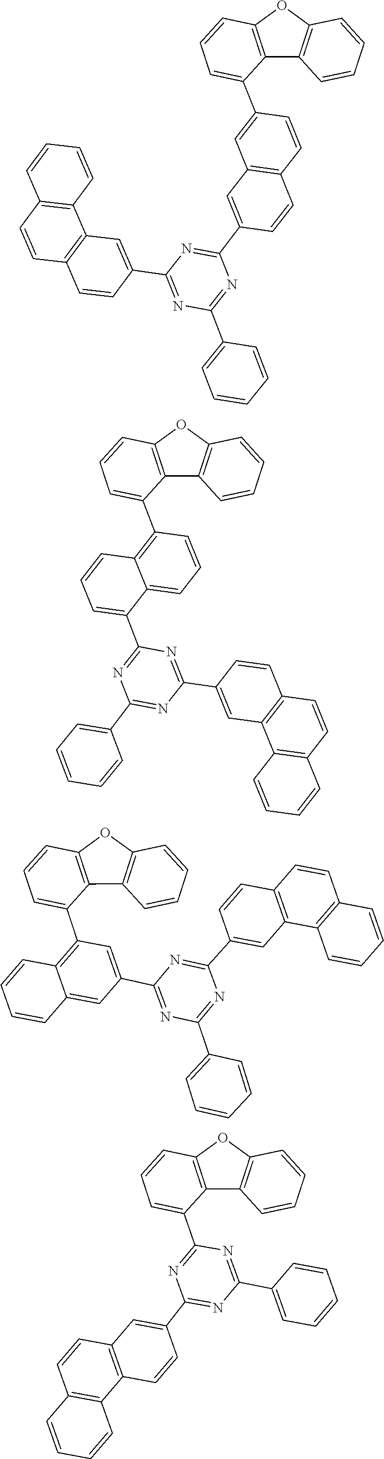

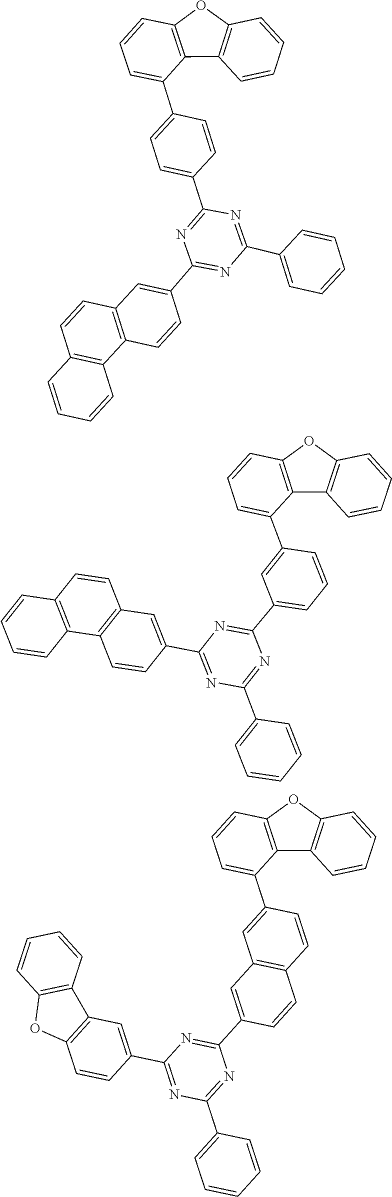

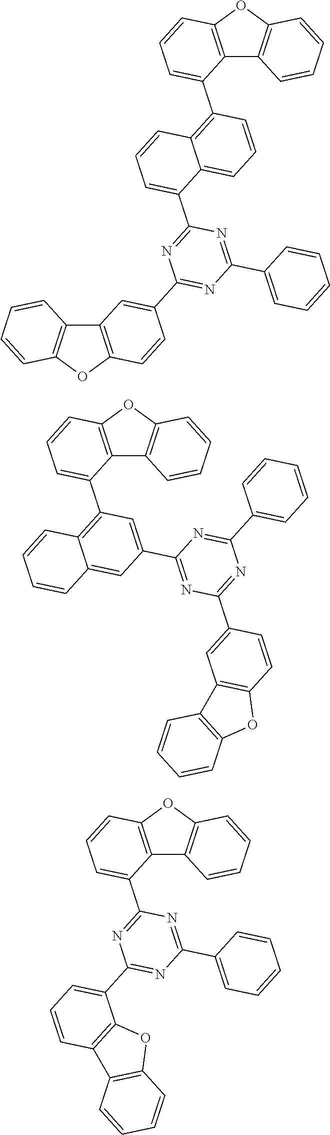

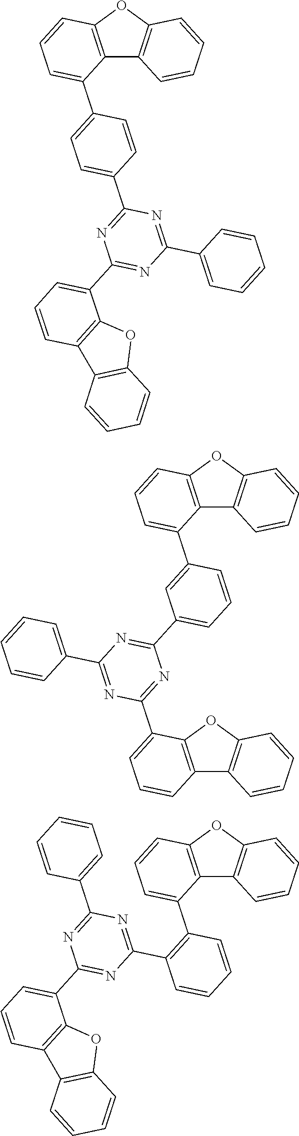

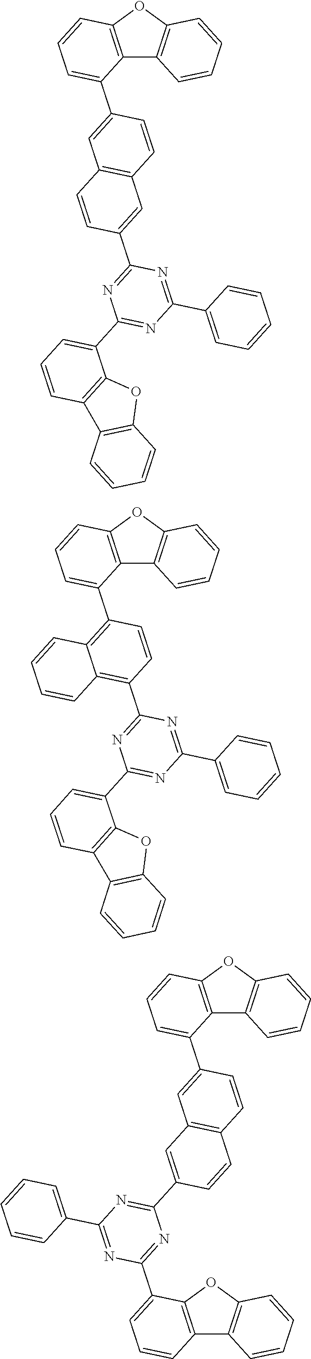

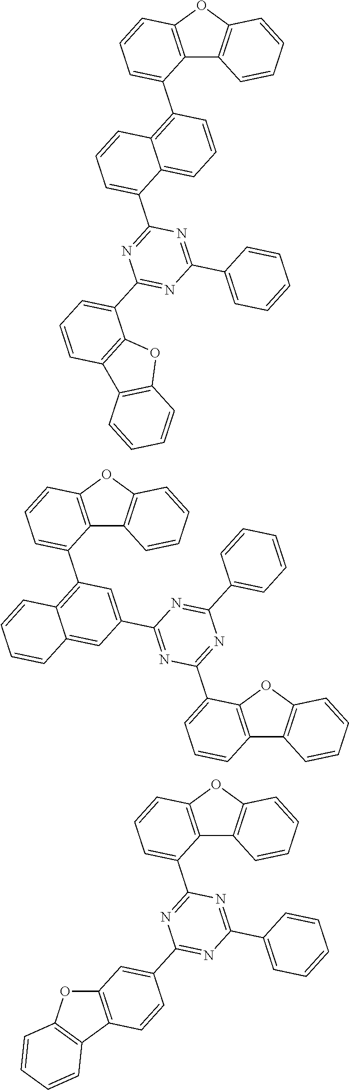

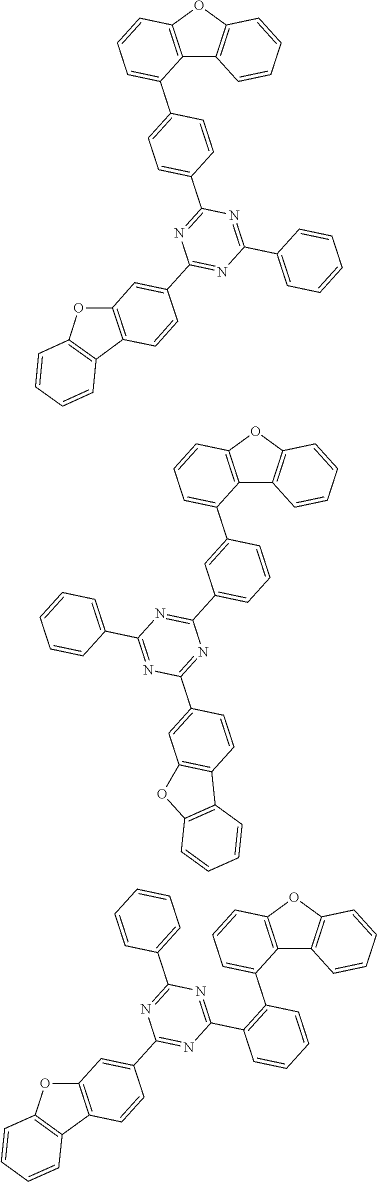

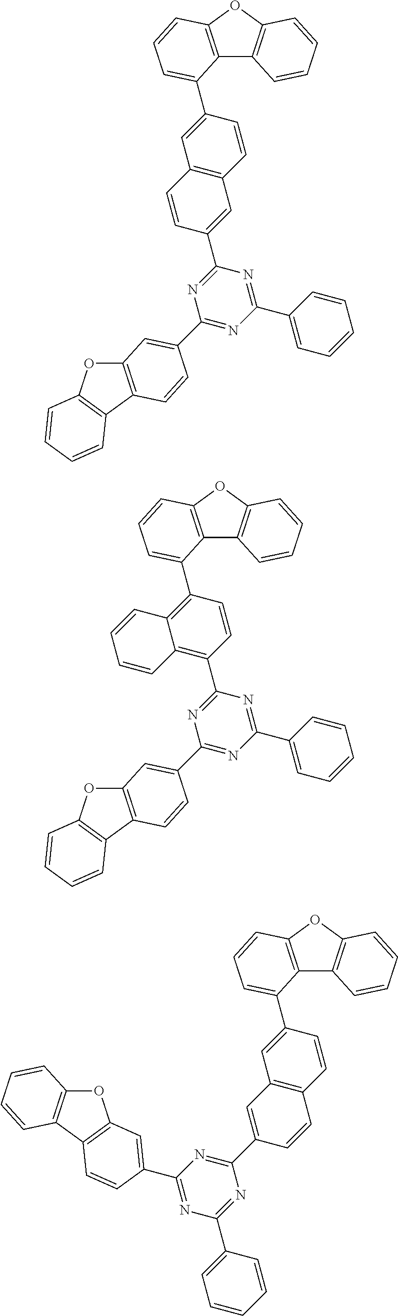 [SMILES: c1ccc(-c2nc(-c3ccc4cc(-c5cccc6oc7ccccc7c56)ccc4c3)nc(-c3ccc4c(c3)oc3ccccc34)n2)cc1 ; c1ccc(-c2nc(-c3ccc4c(c3)oc3ccccc34)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(c3)oc3ccccc34)n2)cc1]

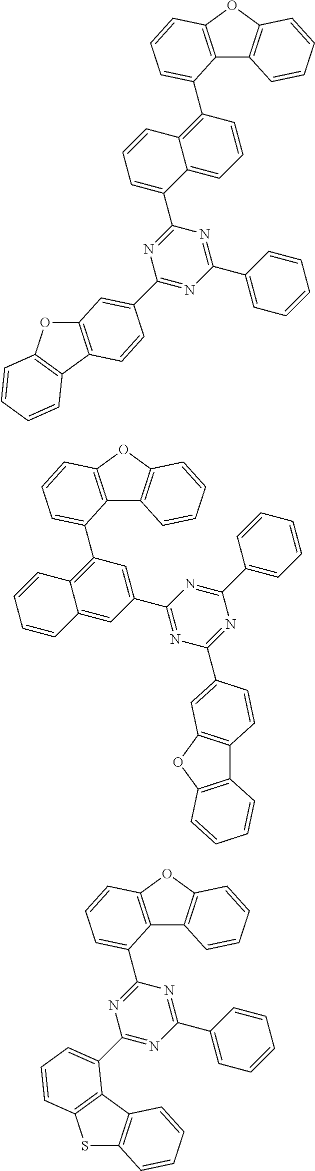

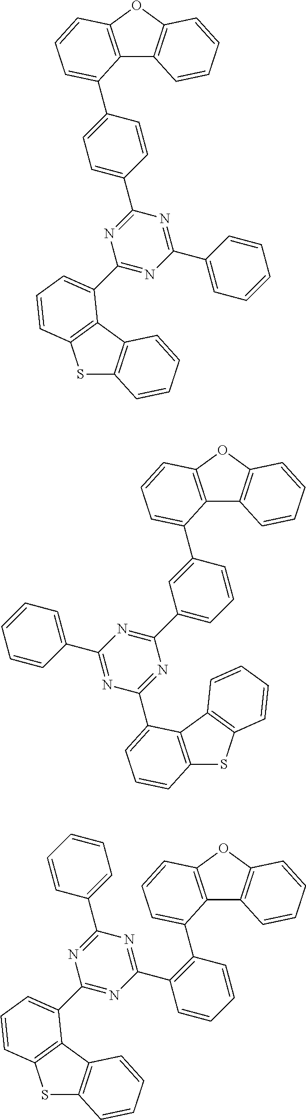 [SMILES: c1ccc(-c2nc(-c3ccc(-c4cccc5oc6ccccc6c45)cc3)nc(-c3cccc4sc5ccccc5c34)n2)cc1 ; c1ccc(-c2nc(-c3cccc(-c4cccc5oc6ccccc6c45)c3)nc(-c3cccc4sc5ccccc5c34)n2)cc1 ; c1ccc(-c2nc(-c3ccccc3-c3cccc4oc5ccccc5c34)nc(-c3cccc4sc5ccccc5c34)n2)cc1]

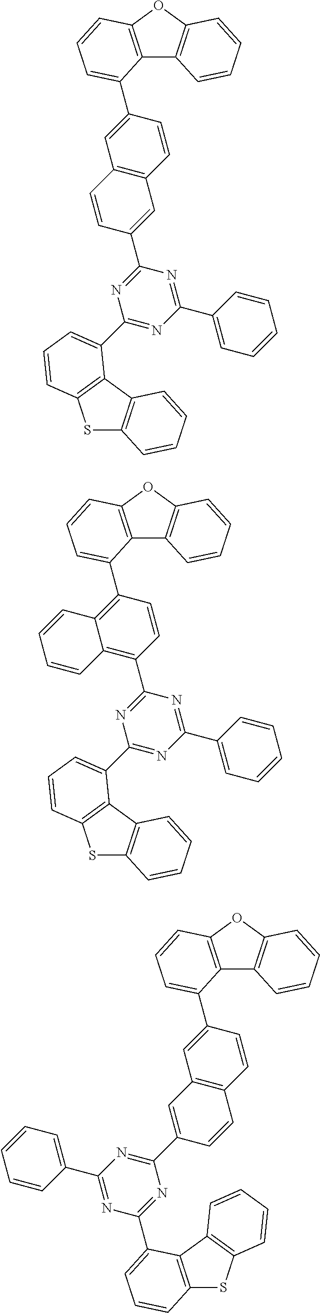

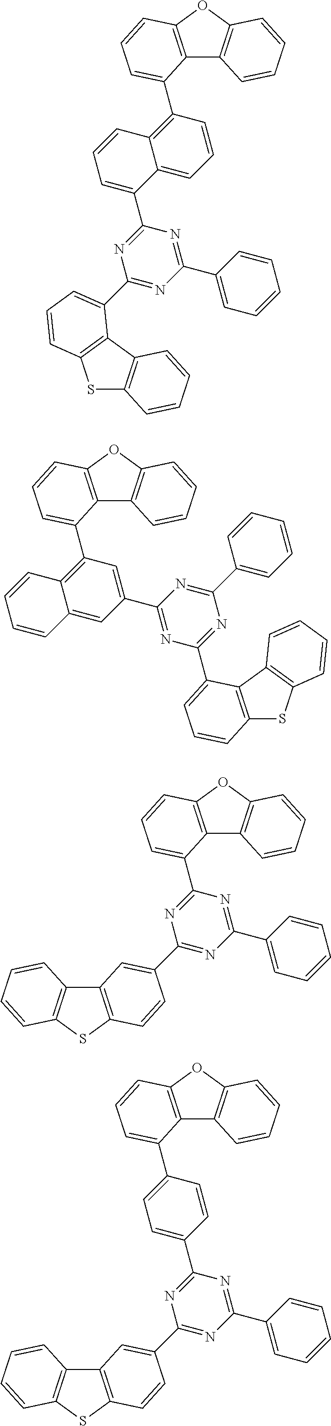

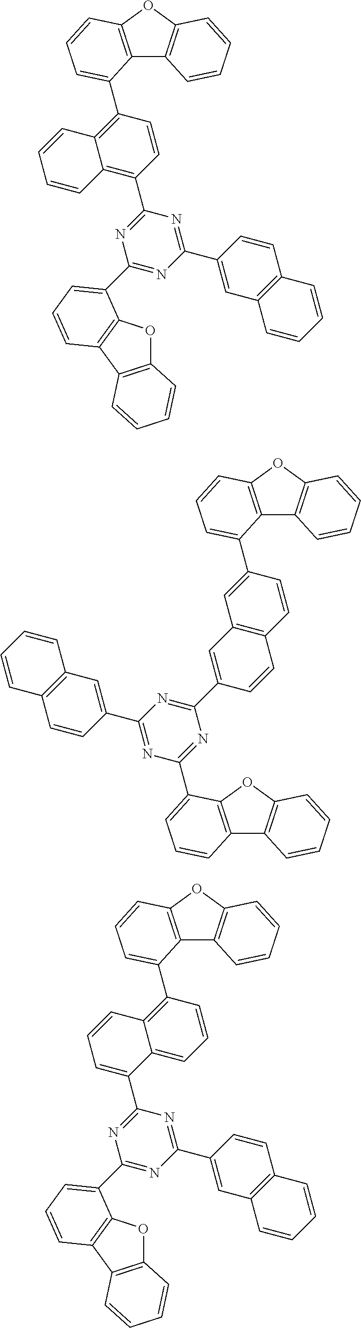

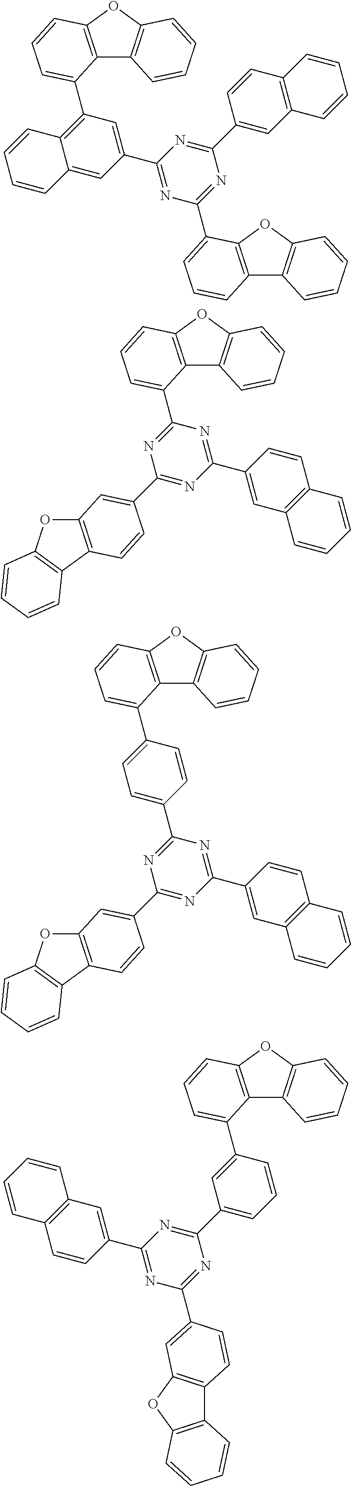

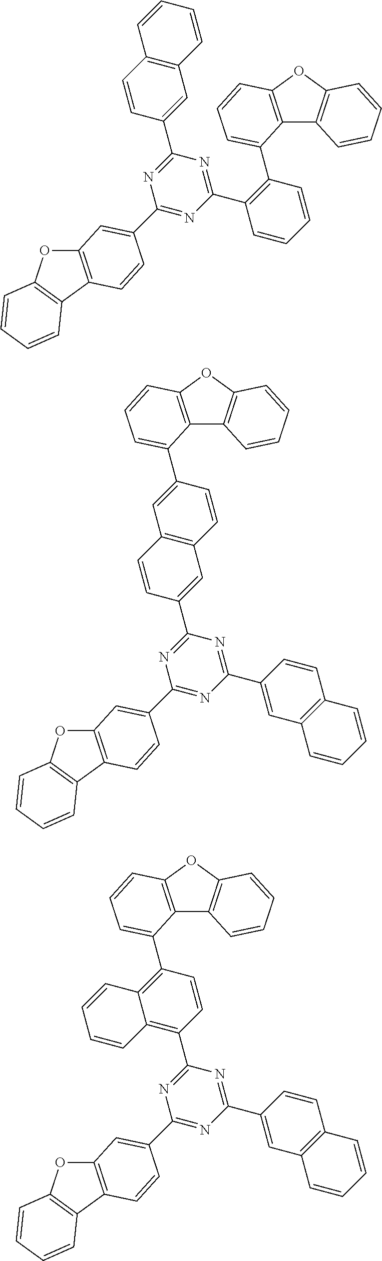 [SMILES: c1ccc(-c2cccc3oc4ccccc4c23)c(-c2nc(-c3ccc4ccccc4c3)nc(-c3ccc4c(c3)oc3ccccc34)n2)c1 ; c1ccc2cc(-c3nc(-c4ccc5cc(-c6cccc7oc8ccccc8c67)ccc5c4)nc(-c4ccc5c(c4)oc4ccccc45)n3)ccc2c1 ; c1ccc2cc(-c3nc(-c4ccc5c(c4)oc4ccccc45)nc(-c4ccc(-c5cccc6oc7ccccc7c56)c5ccccc45)n3)ccc2c1]

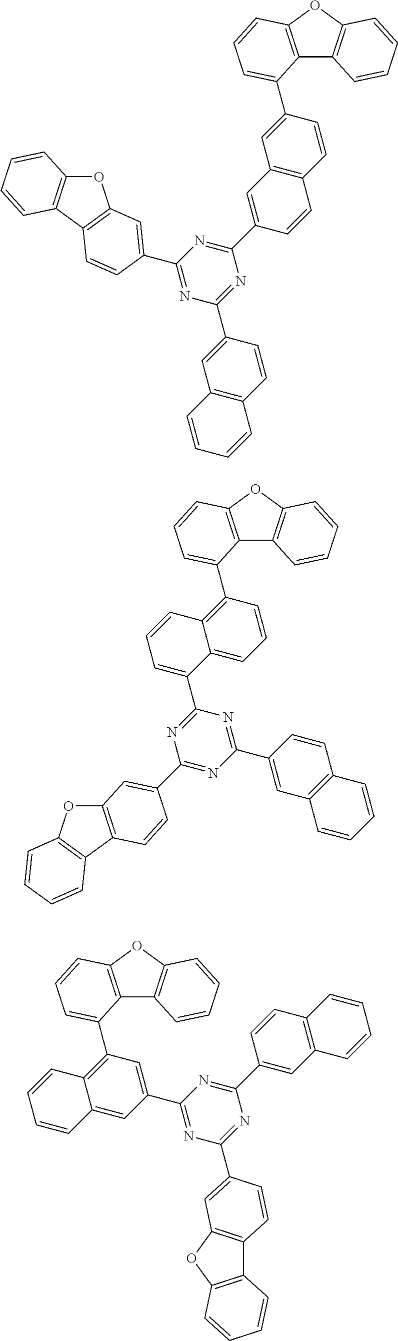

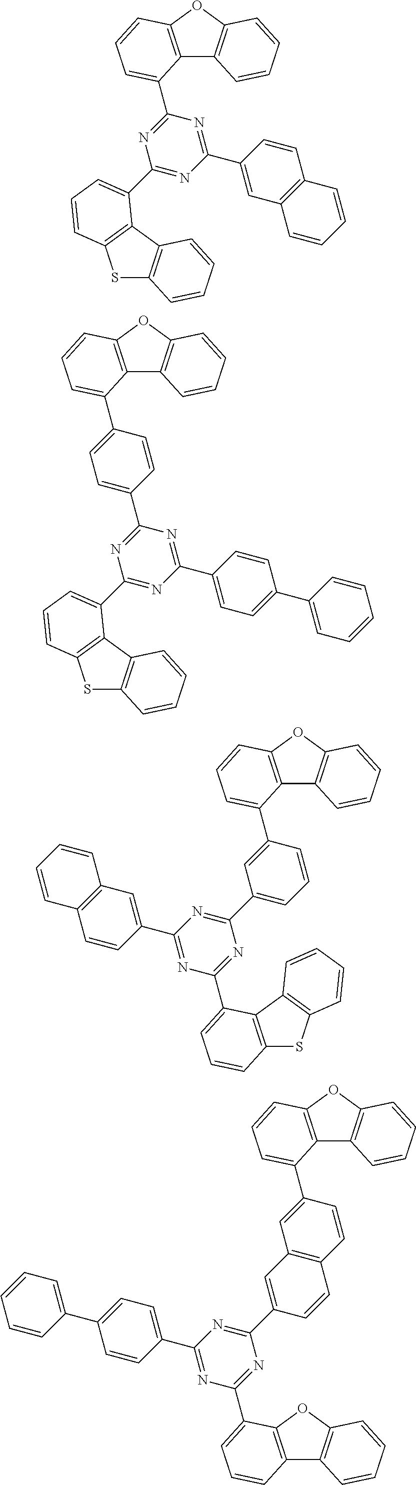

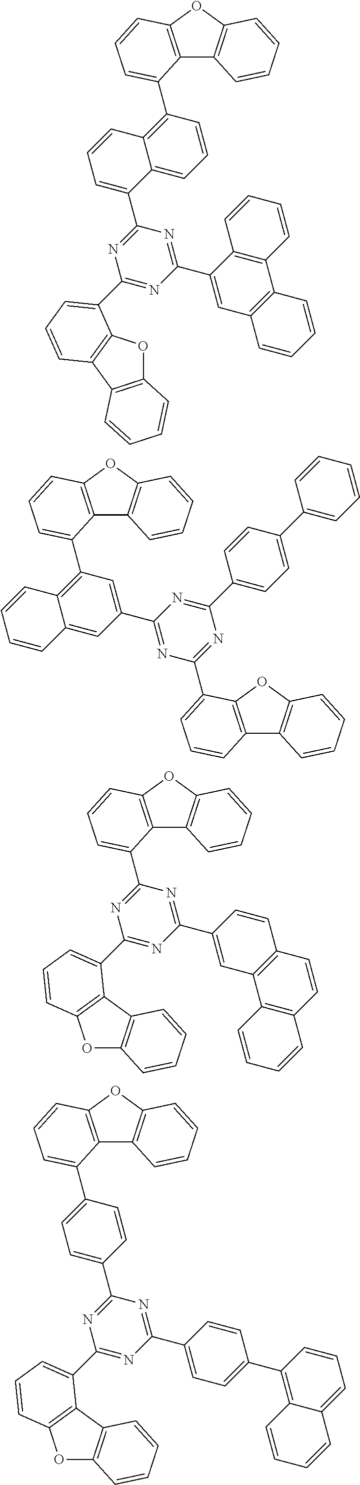

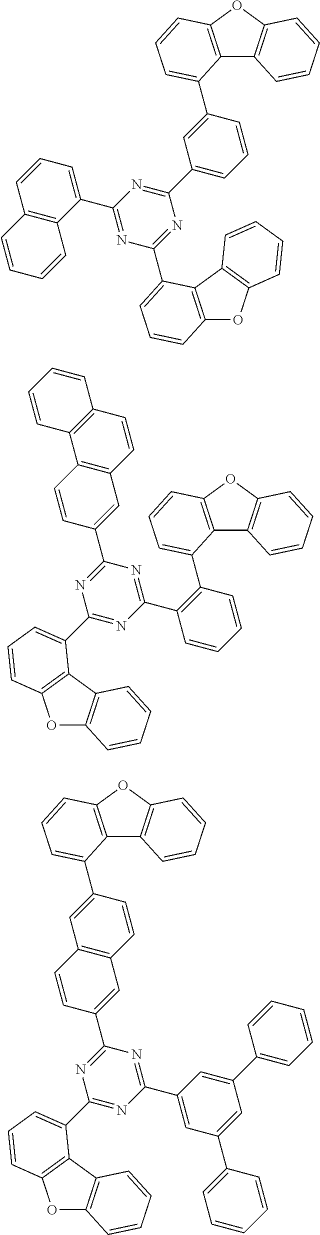

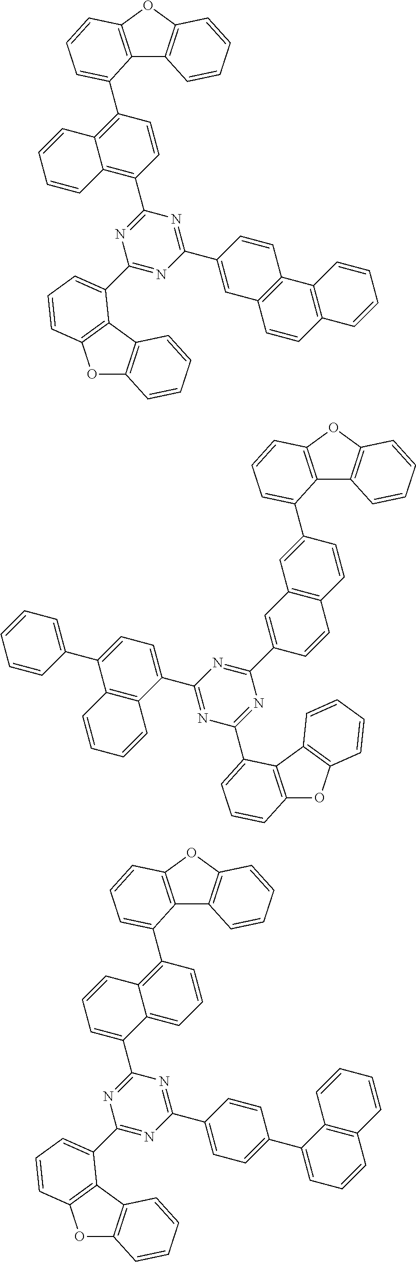

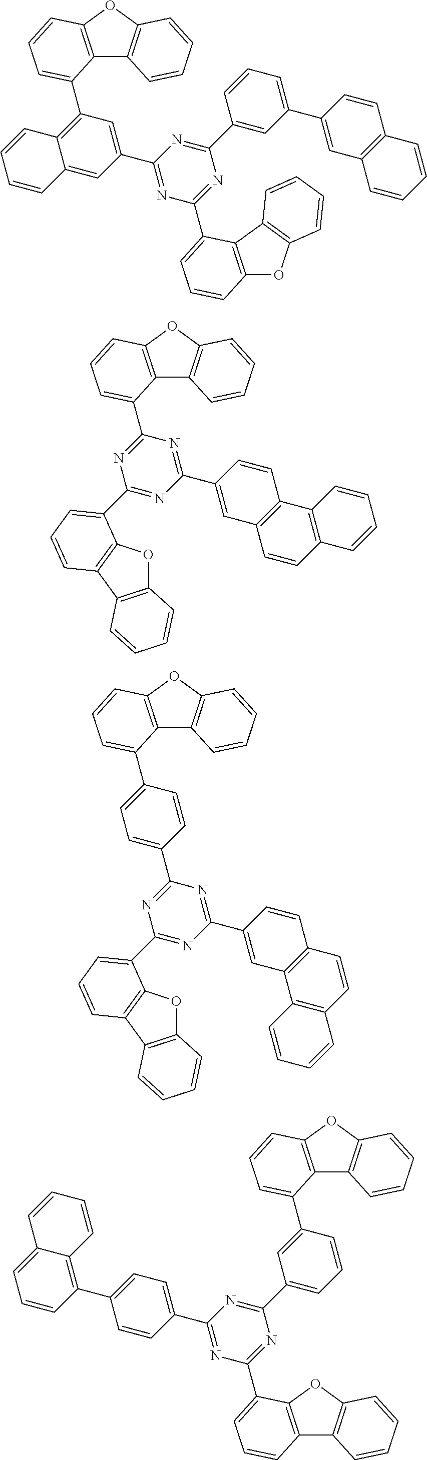

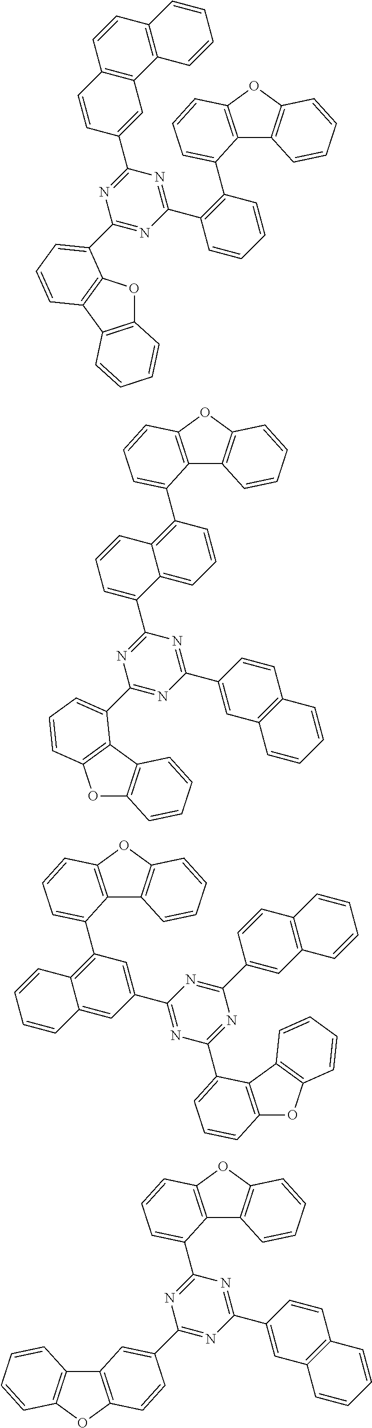

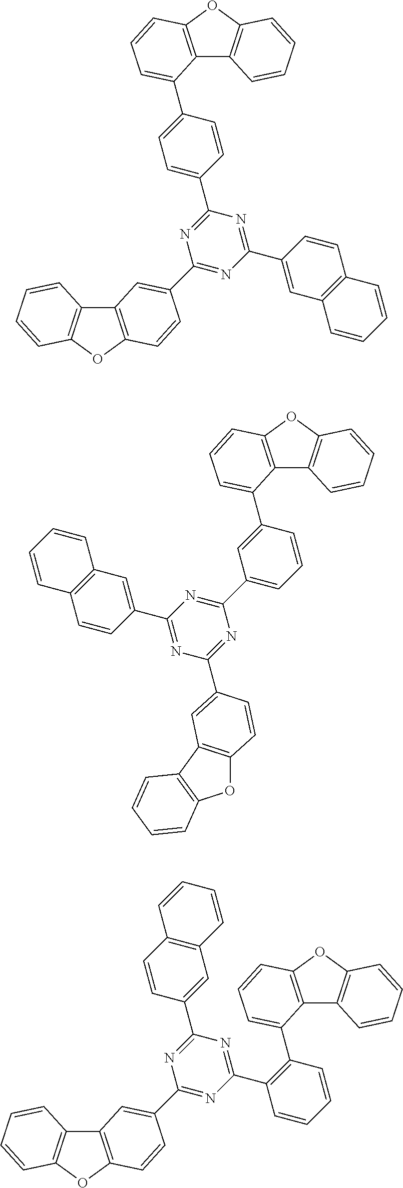 [SMILES: c1ccc2cc(-c3nc(-c4ccc(-c5cccc6oc7ccccc7c56)cc4)nc(-c4ccc5oc6ccccc6c5c4)n3)ccc2c1 ; c1cc(-c2nc(-c3ccc4ccccc4c3)nc(-c3ccc4oc5ccccc5c4c3)n2)cc(-c2cccc3oc4ccccc4c23)c1 ; c1ccc(-c2cccc3oc4ccccc4c23)c(-c2nc(-c3ccc4ccccc4c3)nc(-c3ccc4oc5ccccc5c4c3)n2)c1]

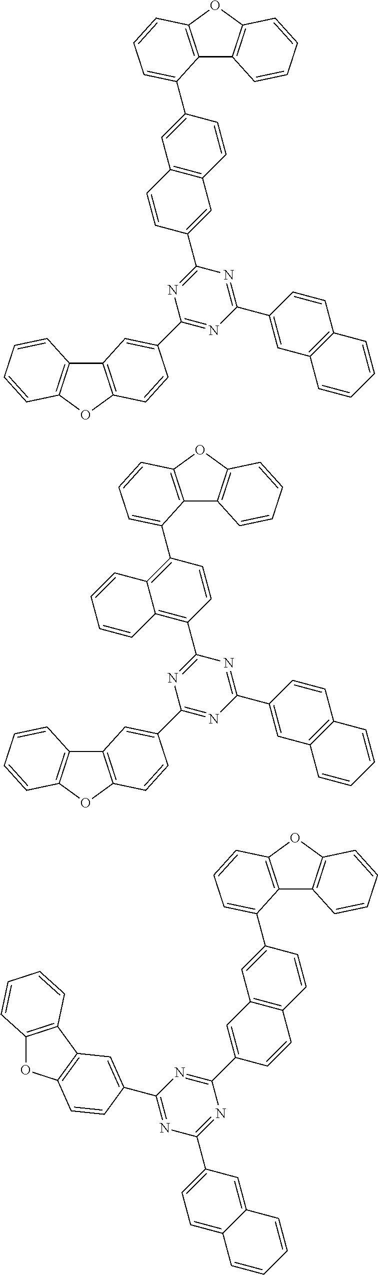 [SMILES: c1ccc2cc(-c3nc(-c4ccc5cc(-c6cccc7oc8ccccc8c67)ccc5c4)nc(-c4ccc5oc6ccccc6c5c4)n3)ccc2c1 ; c1ccc2cc(-c3nc(-c4ccc5oc6ccccc6c5c4)nc(-c4ccc(-c5cccc6oc7ccccc7c56)c5ccccc45)n3)ccc2c1 ; c1ccc2cc(-c3nc(-c4ccc5ccc(-c6cccc7oc8ccccc8c67)cc5c4)nc(-c4ccc5oc6ccccc6c5c4)n3)ccc2c1]

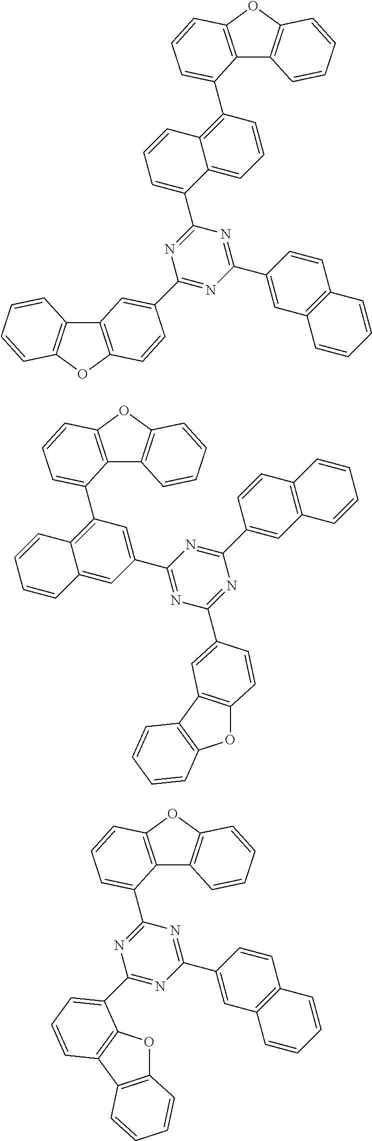

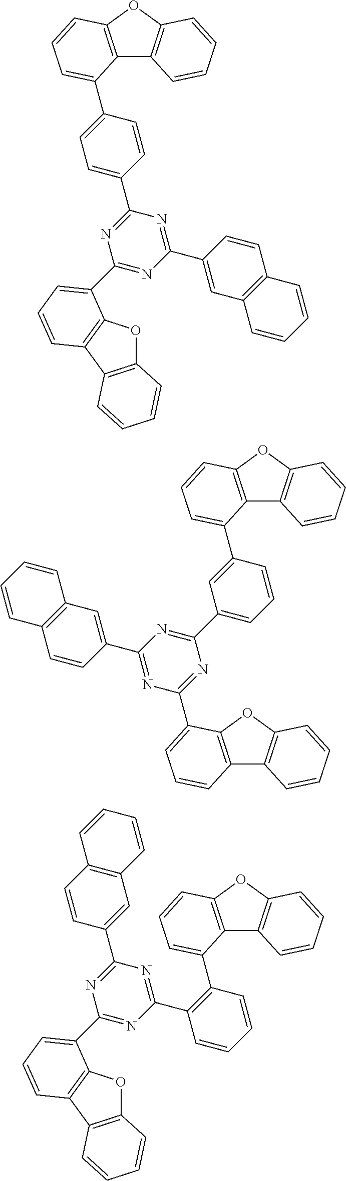 [SMILES: c1ccc2cc(-c3nc(-c4ccc(-c5cccc6oc7ccccc7c56)cc4)nc(-c4cccc5c4oc4ccccc45)n3)ccc2c1 ; c1cc(-c2nc(-c3ccc4ccccc4c3)nc(-c3cccc4c3oc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3cccc4c3oc3ccccc34)n2)c1]

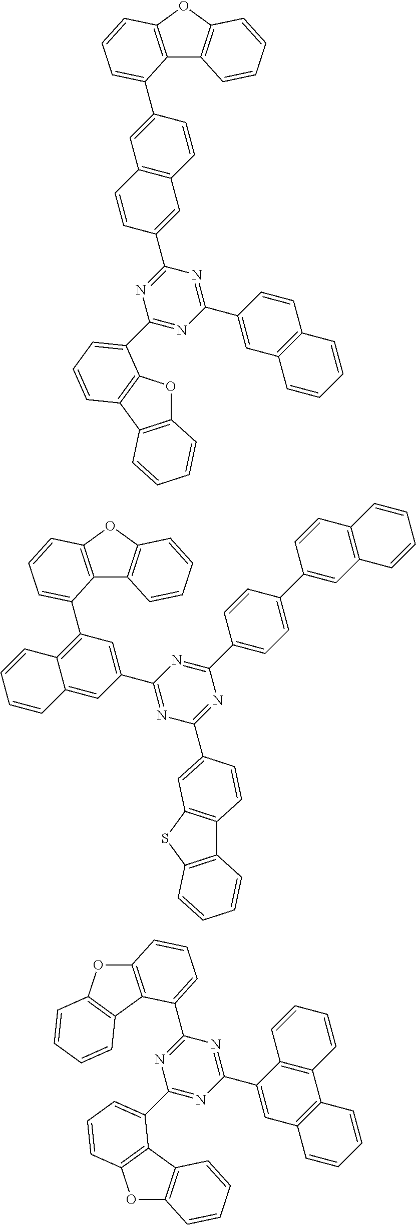

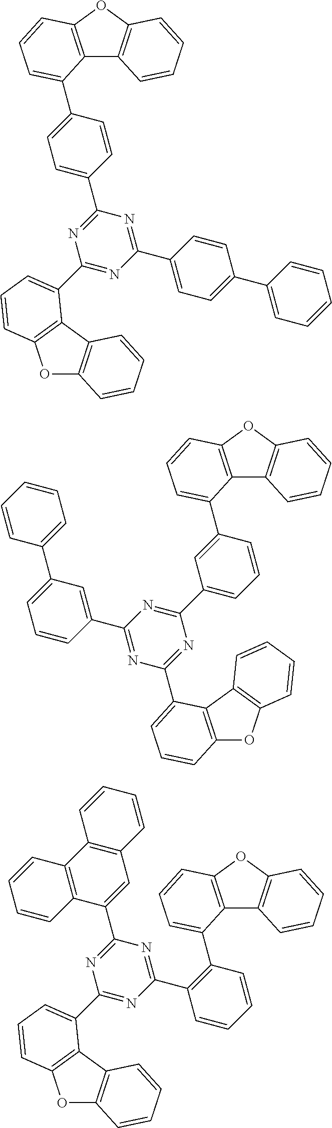

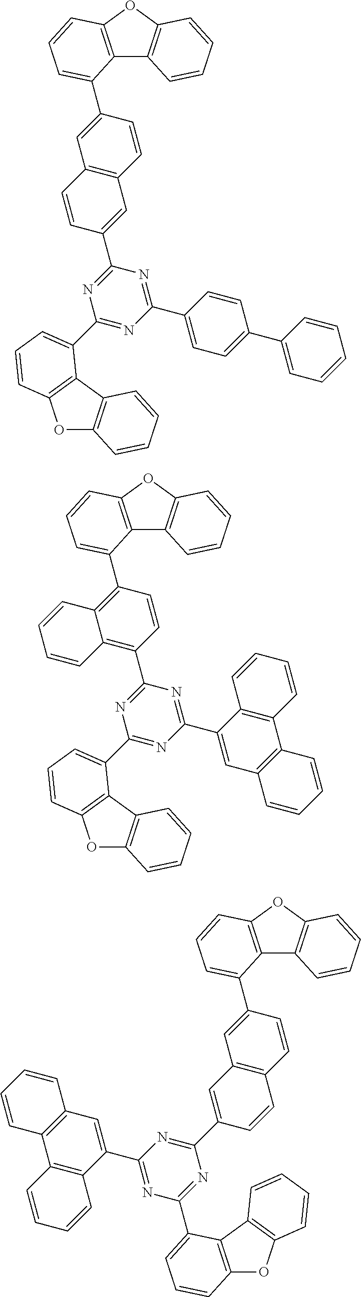

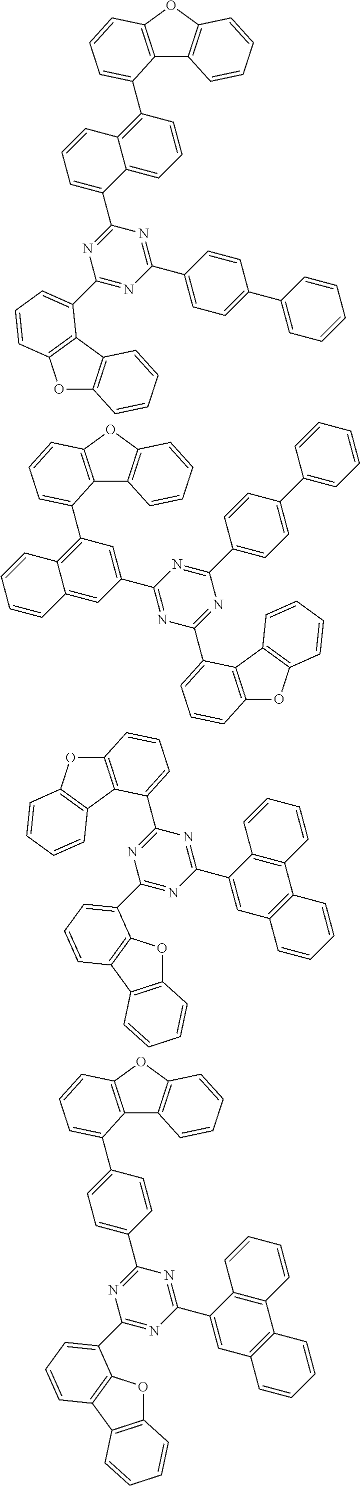

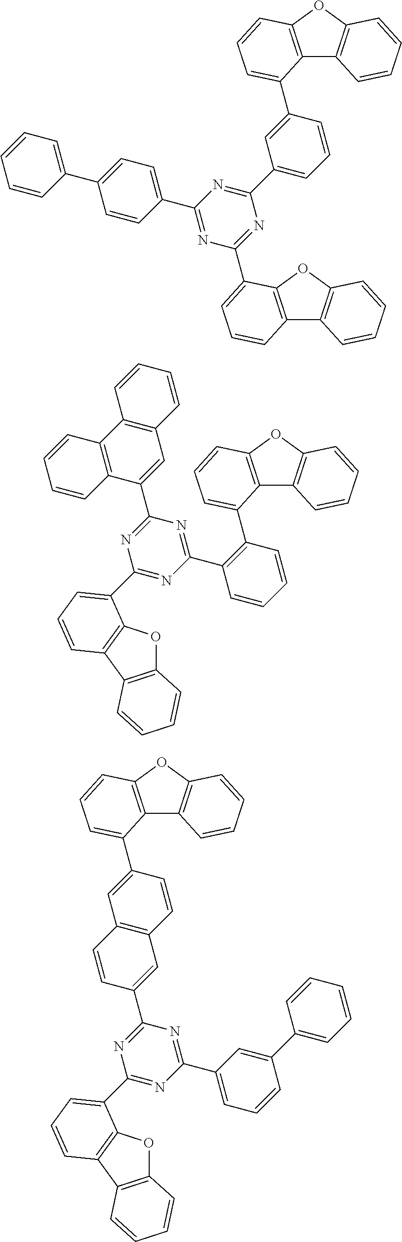

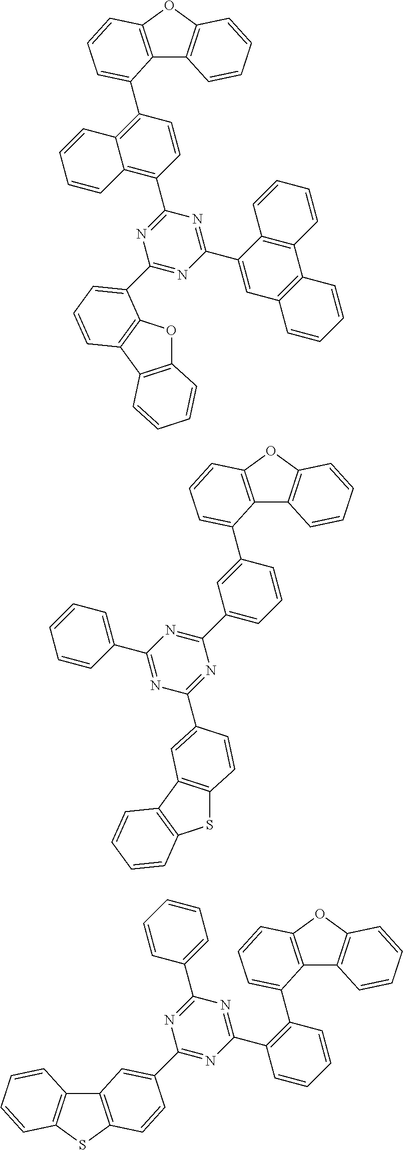

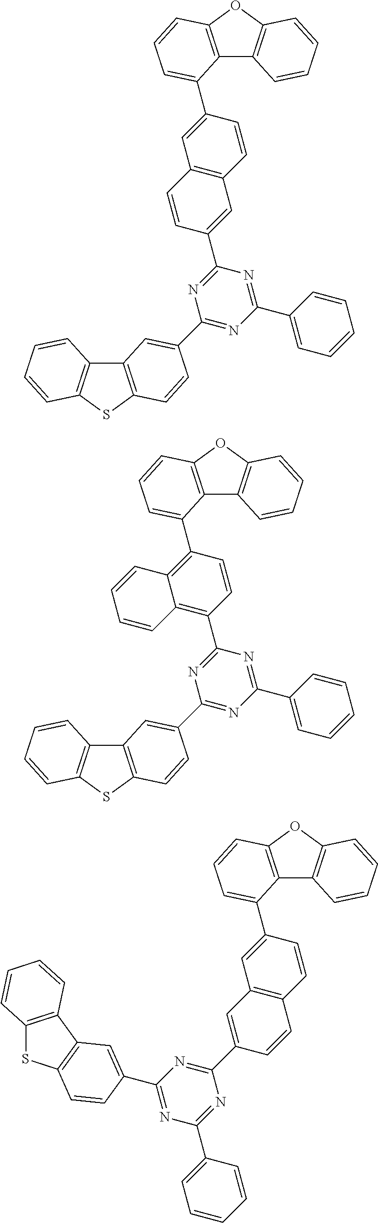 [SMILES: c1ccc(-c2nc(-c3ccc4cc(-c5cccc6oc7ccccc7c56)ccc4c3)nc(-c3ccc4sc5ccccc5c4c3)n2)cc1 ; c1ccc(-c2nc(-c3ccc4sc5ccccc5c4c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4sc5ccccc5c4c3)n2)cc1]

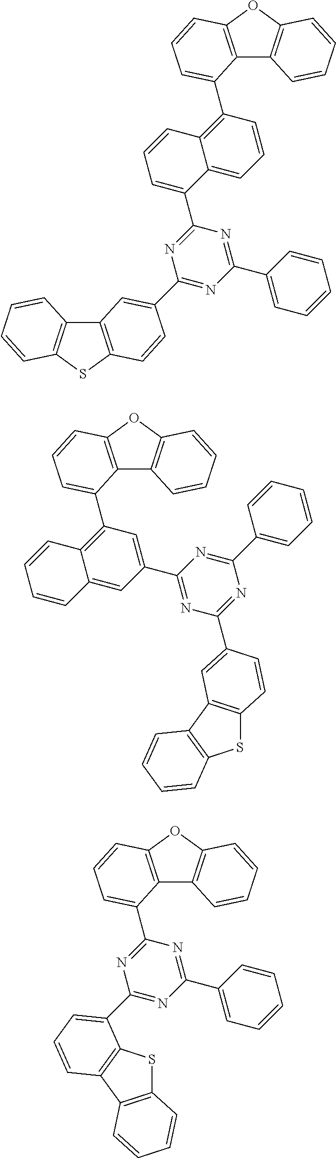

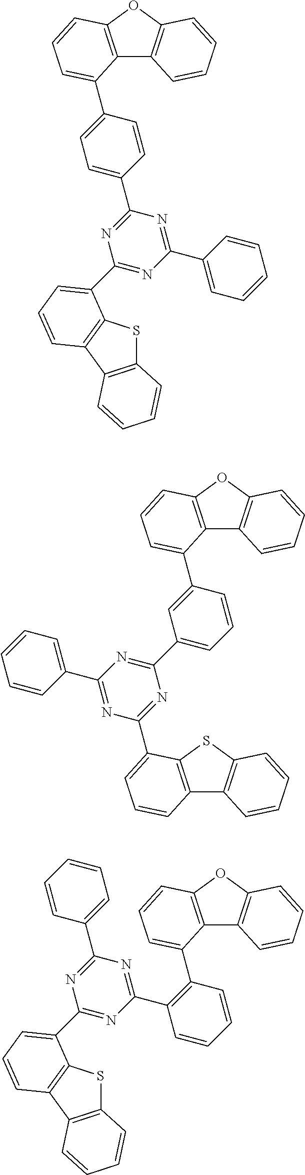 [SMILES: c1ccc(-c2nc(-c3ccc(-c4cccc5oc6ccccc6c45)cc3)nc(-c3cccc4c3sc3ccccc34)n2)cc1 ; c1ccc(-c2nc(-c3cccc(-c4cccc5oc6ccccc6c45)c3)nc(-c3cccc4c3sc3ccccc34)n2)cc1 ; c1ccc(-c2nc(-c3ccccc3-c3cccc4oc5ccccc5c34)nc(-c3cccc4c3sc3ccccc34)n2)cc1]

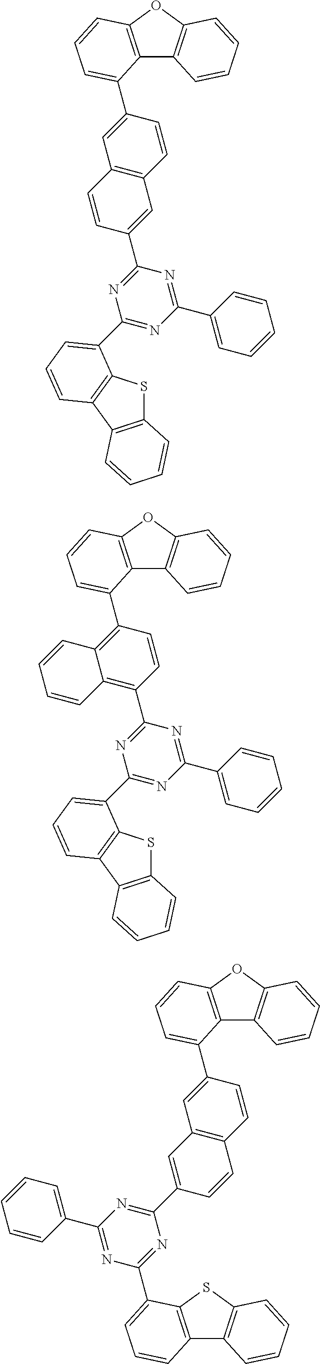

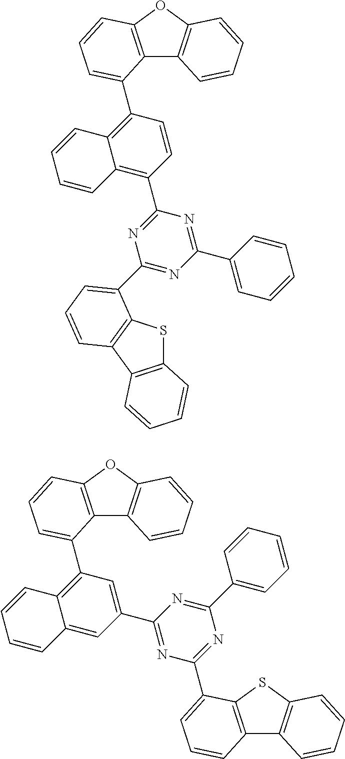 [SMILES: c1ccc(-c2nc(-c3ccc(-c4cccc5oc6ccccc6c45)c4ccccc34)nc(-c3cccc4c3sc3ccccc34)n2)cc1 ; c1ccc(-c2nc(-c3cc(-c4cccc5oc6ccccc6c45)c4ccccc4c3)nc(-c3cccc4c3sc3ccccc34)n2)cc1]

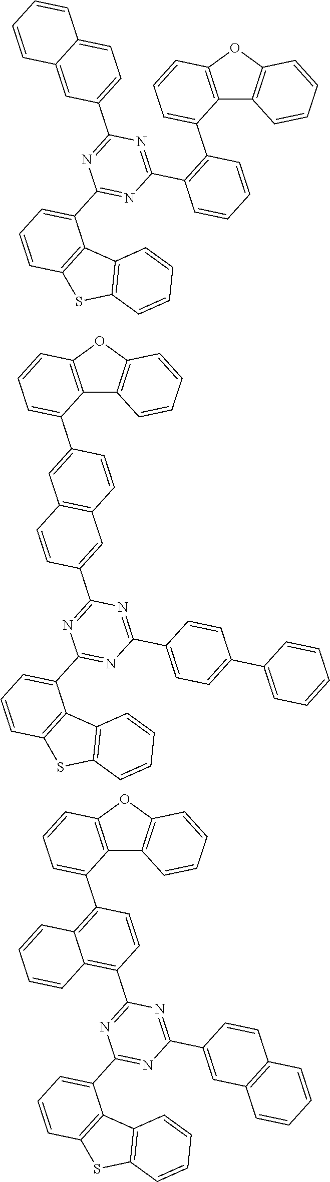

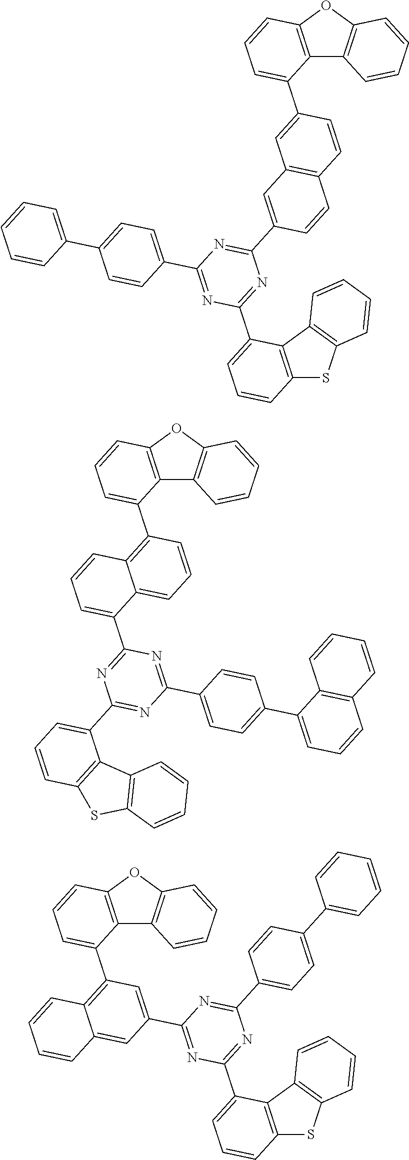

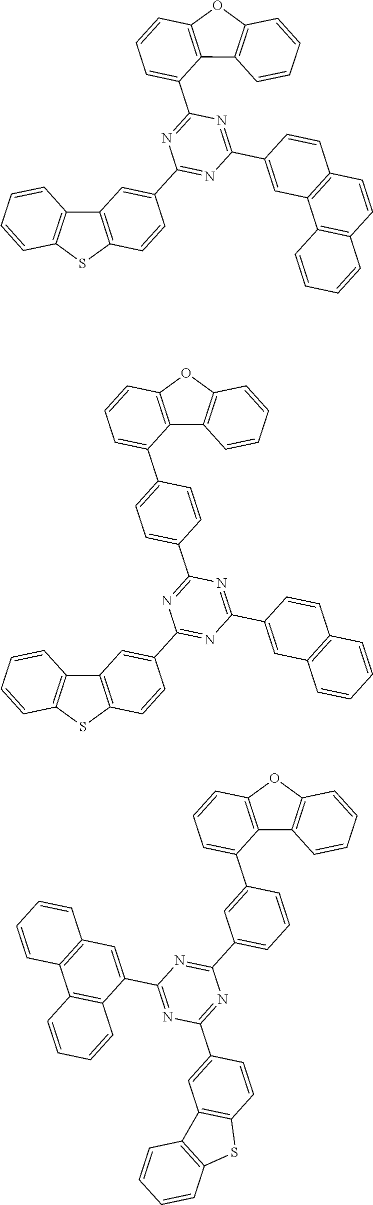 [SMILES: c1ccc2c(c1)ccc1ccc(-c3nc(-c4ccc5sc6ccccc6c5c4)nc(-c4cccc5oc6ccccc6c45)n3)cc12 ; c1ccc2cc(-c3nc(-c4ccc(-c5cccc6oc7ccccc7c56)cc4)nc(-c4ccc5sc6ccccc6c5c4)n3)ccc2c1 ; c1cc(-c2nc(-c3ccc4sc5ccccc5c4c3)nc(-c3cc4ccccc4c4ccccc34)n2)cc(-c2cccc3oc4ccccc4c23)c1]

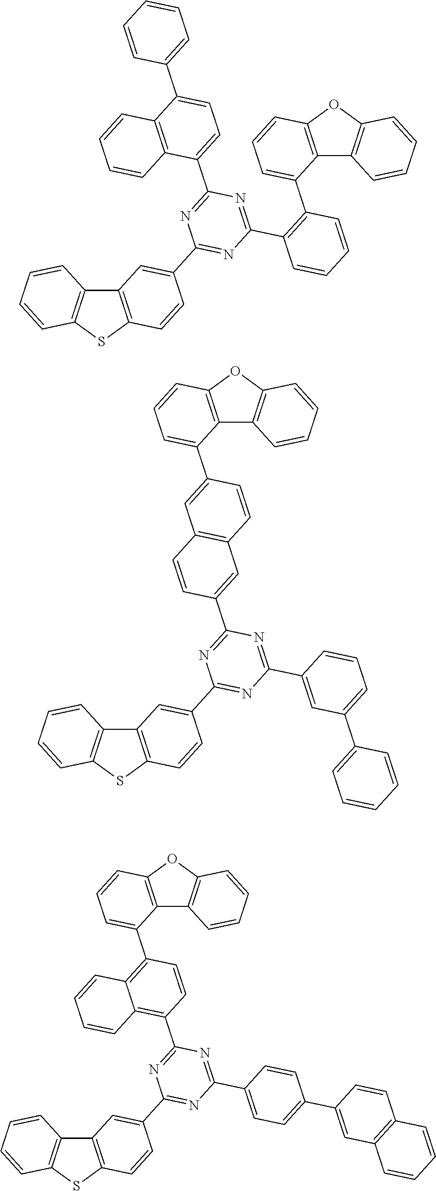 [SMILES: c1ccc(-c2ccc(-c3nc(-c4ccc5sc6ccccc6c5c4)nc(-c4ccccc4-c4cccc5oc6ccccc6c45)n3)c3ccccc23)cc1 ; c1ccc(-c2cccc(-c3nc(-c4ccc5cc(-c6cccc7oc8ccccc8c67)ccc5c4)nc(-c4ccc5sc6ccccc6c5c4)n3)c2)cc1 ; c1ccc2cc(-c3ccc(-c4nc(-c5ccc6sc7ccccc7c6c5)nc(-c5ccc(-c6cccc7oc8ccccc8c67)c6ccccc56)n4)cc3)ccc2c1]

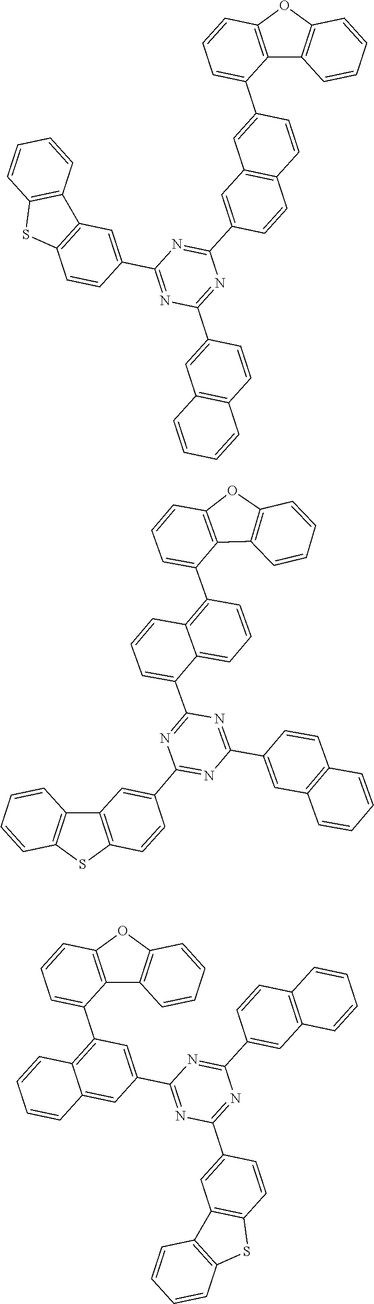

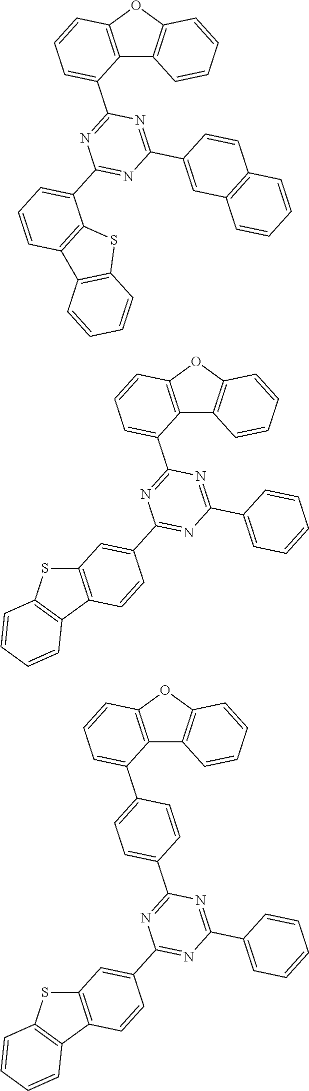 [SMILES: c1ccc2cc(-c3nc(-c4cccc5c4sc4ccccc45)nc(-c4cccc5oc6ccccc6c45)n3)ccc2c1 ; c1ccc(-c2nc(-c3ccc4c(c3)sc3ccccc34)nc(-c3cccc4oc5ccccc5c34)n2)cc1 ; c1ccc(-c2nc(-c3ccc(-c4cccc5oc6ccccc6c45)cc3)nc(-c3ccc4c(c3)sc3ccccc34)n2)cc1]

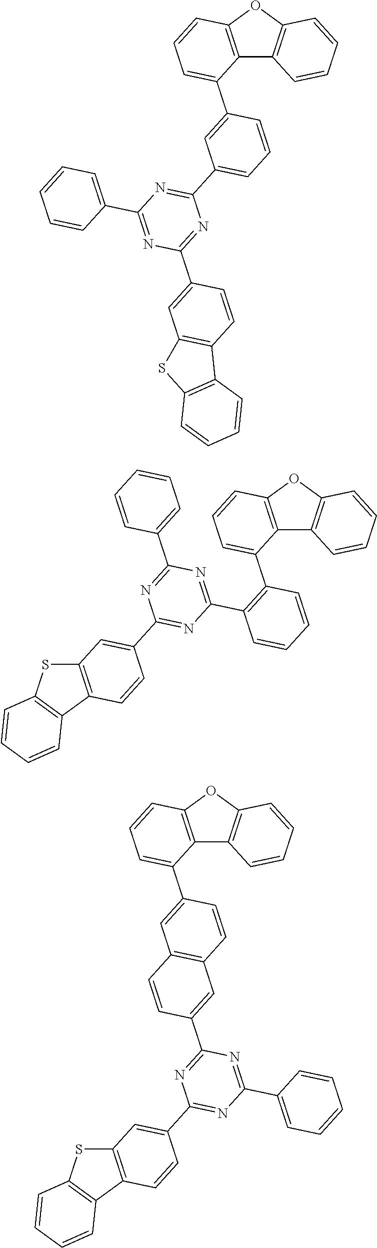 [SMILES: c1ccc(-c2nc(-c3cccc(-c4cccc5oc6ccccc6c45)c3)nc(-c3ccc4c(c3)sc3ccccc34)n2)cc1 ; c1ccc(-c2nc(-c3ccc4c(c3)sc3ccccc34)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(c3)sc3ccccc34)n2)cc1]

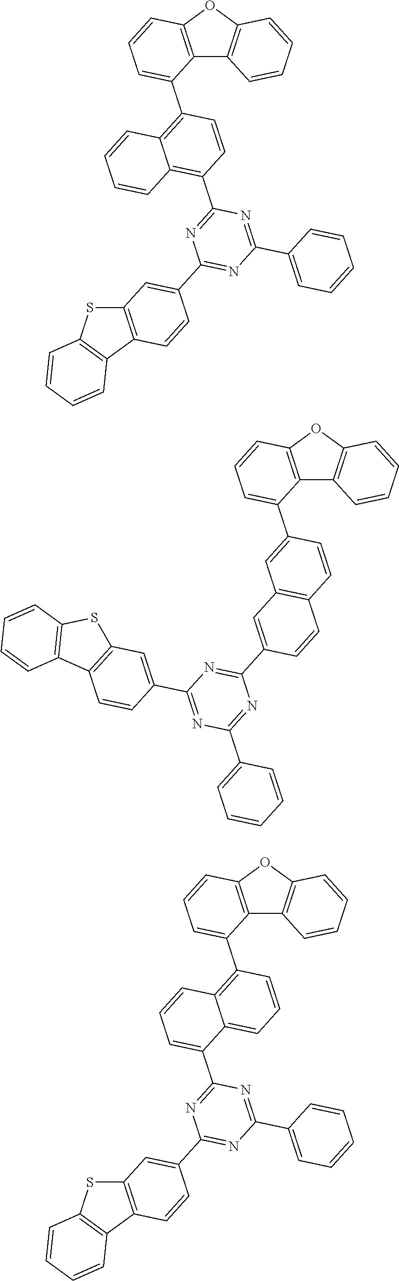

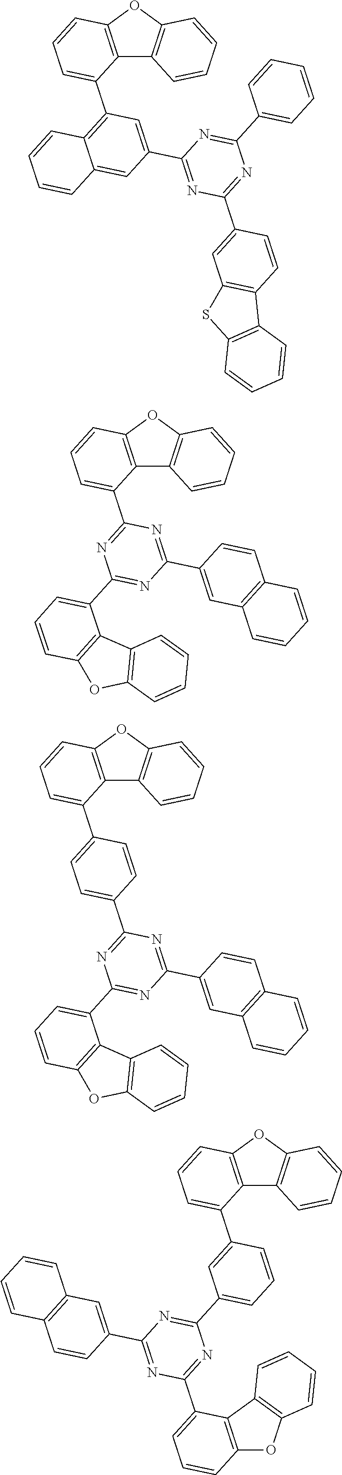

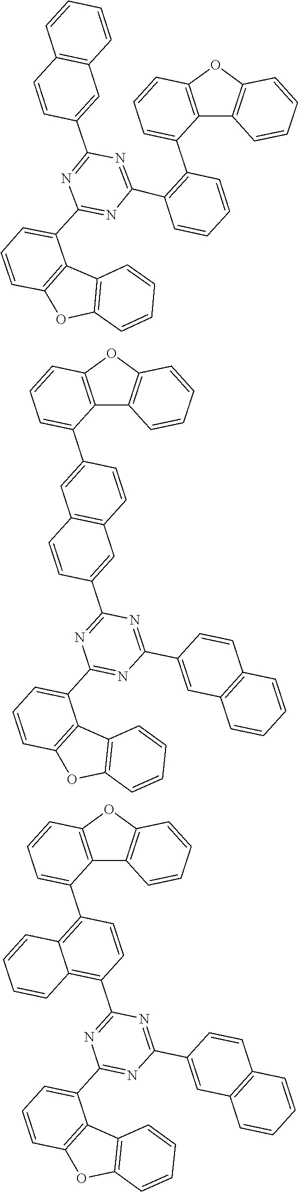

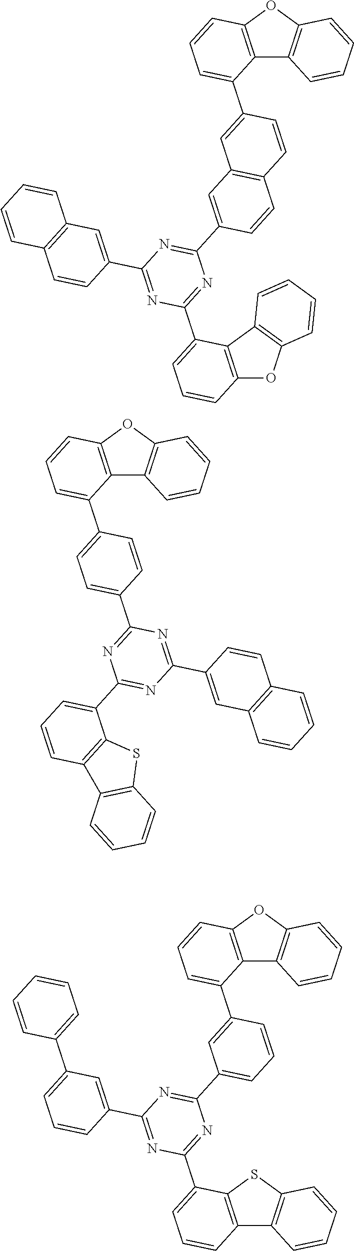

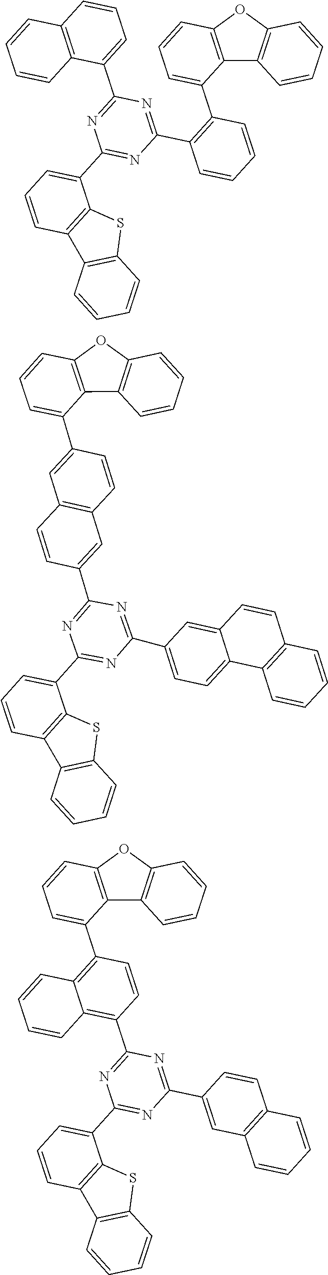

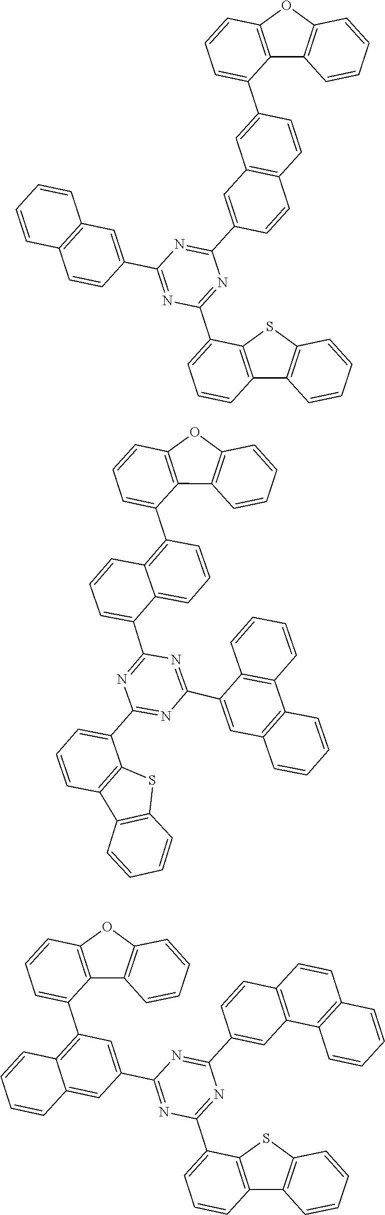

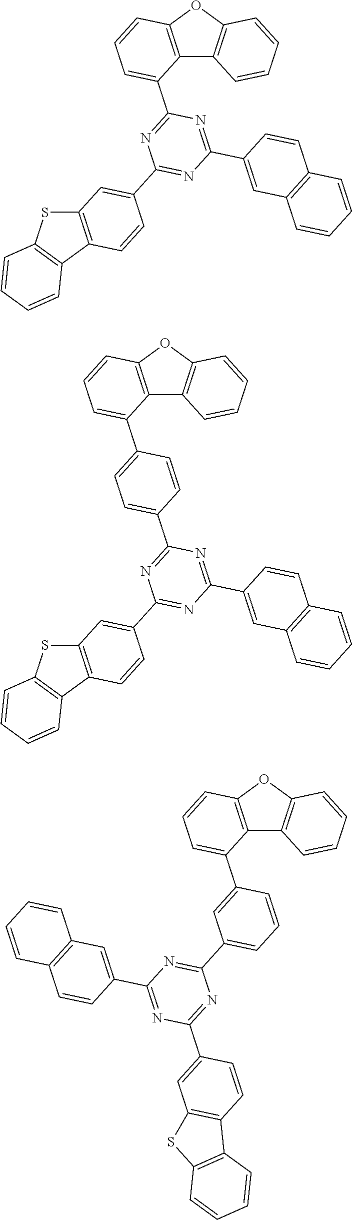 [SMILES: c1ccc2cc(-c3nc(-c4ccc5c(c4)sc4ccccc45)nc(-c4cccc5oc6ccccc6c45)n3)ccc2c1 ; c1ccc2cc(-c3nc(-c4ccc(-c5cccc6oc7ccccc7c56)cc4)nc(-c4ccc5c(c4)sc4ccccc45)n3)ccc2c1 ; c1cc(-c2nc(-c3ccc4ccccc4c3)nc(-c3ccc4c(c3)sc3ccccc34)n2)cc(-c2cccc3oc4ccccc4c23)c1]

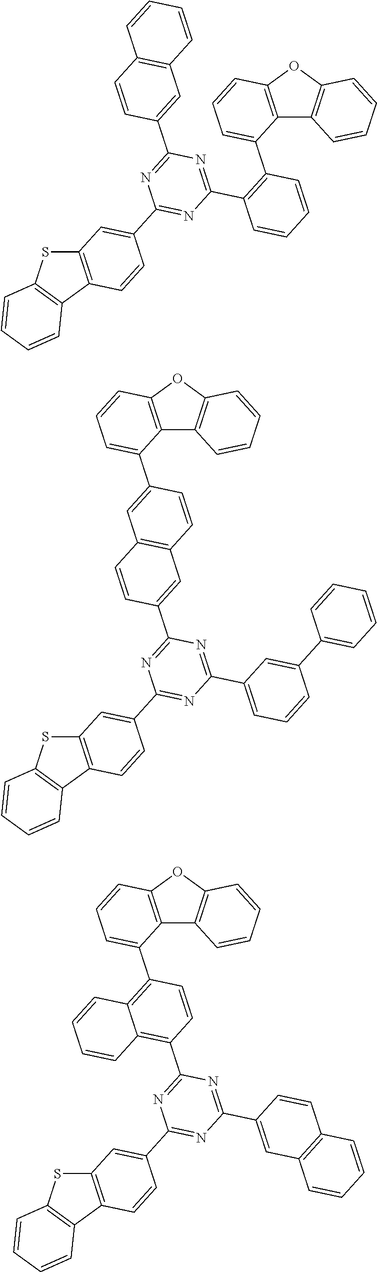 [SMILES: c1ccc(-c2cccc3oc4ccccc4c23)c(-c2nc(-c3ccc4ccccc4c3)nc(-c3ccc4c(c3)sc3ccccc34)n2)c1 ; c1ccc(-c2cccc(-c3nc(-c4ccc5cc(-c6cccc7oc8ccccc8c67)ccc5c4)nc(-c4ccc5c(c4)sc4ccccc45)n3)c2)cc1 ; c1ccc2cc(-c3nc(-c4ccc5c(c4)sc4ccccc45)nc(-c4ccc(-c5cccc6oc7ccccc7c56)c5ccccc45)n3)ccc2c1]

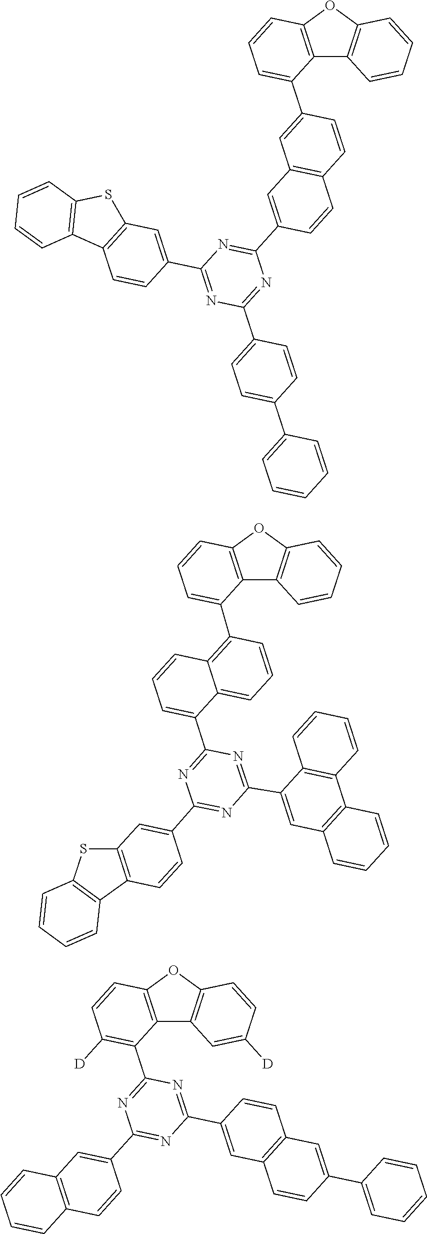

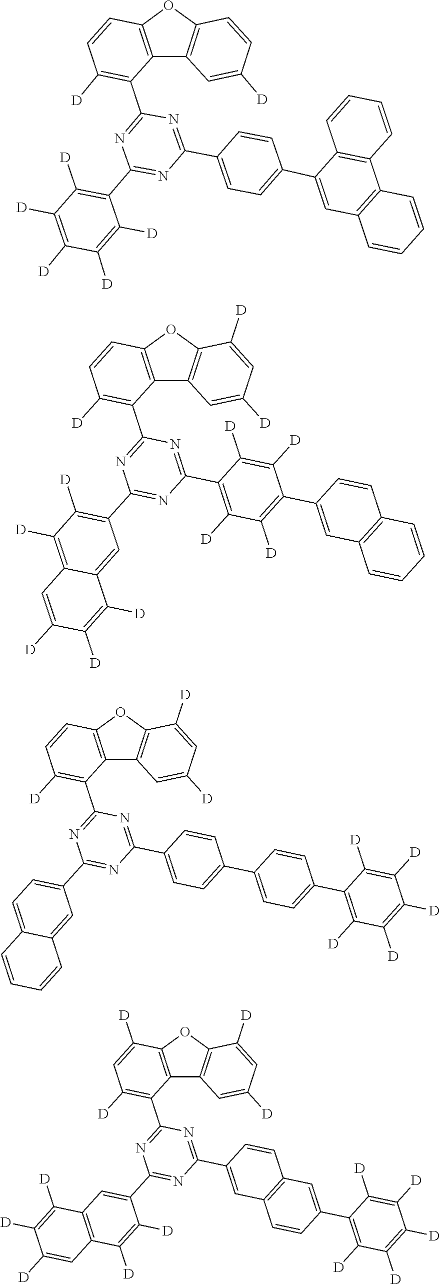

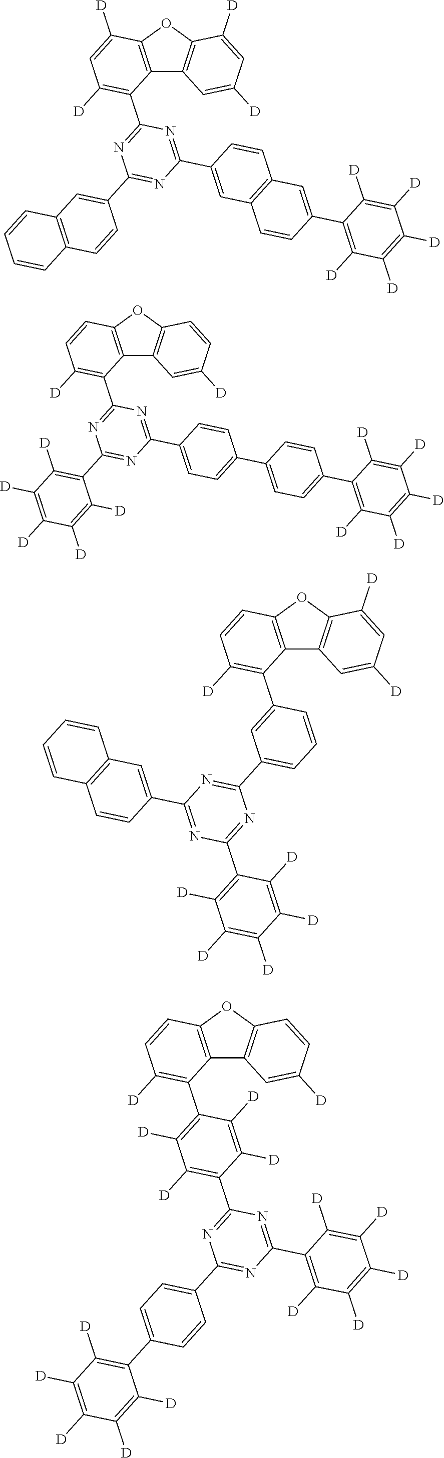

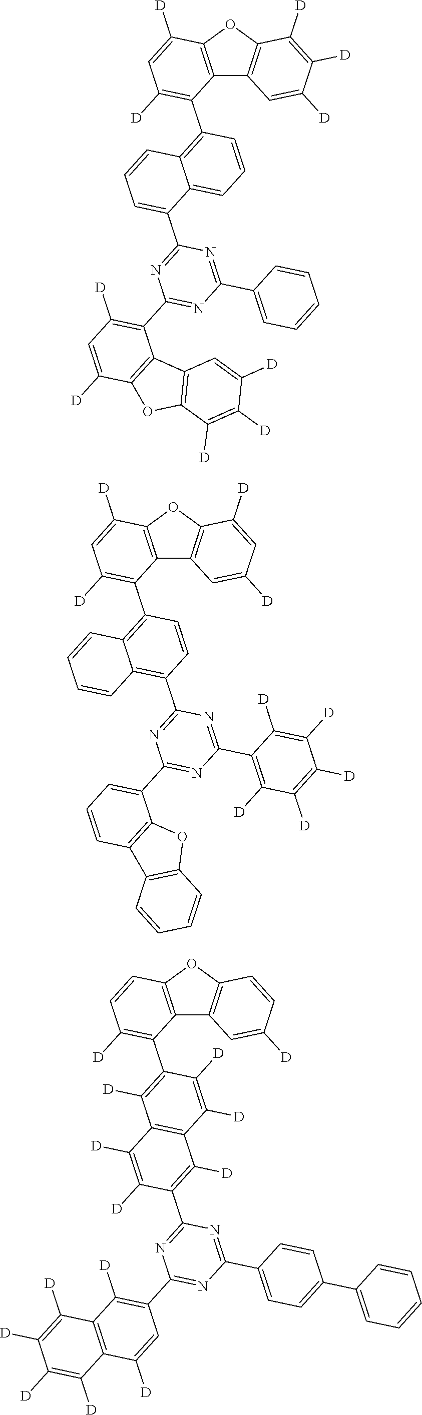

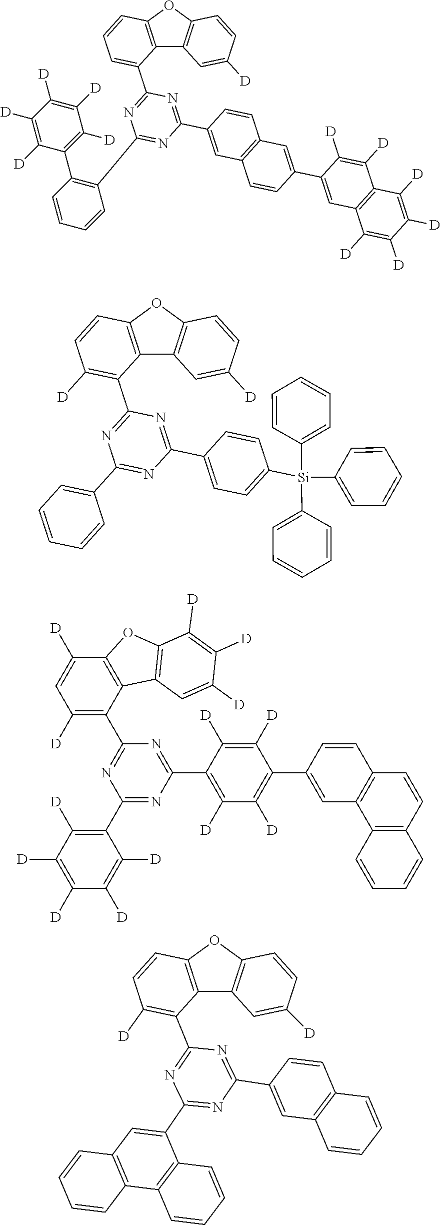 [SMILES: [2H]c1ccc2oc3cccc(-c4nc(-c5ccc6cc(-c7cc8c([2H])c([2H])c([2H])c([2H])c8c([2H])c7[2H])ccc6c5)nc(-c5ccccc5-c5c([2H])c([2H])c([2H])c([2H])c5[2H])n4)c3c2c1 ; [2H]c1ccc2oc3ccc([2H])c(-c4nc(-c5ccccc5)nc(-c5ccc([Si](c6ccccc6)(c6ccccc6)c6ccccc6)cc5)n4)c3c2c1 ; [2H]c1cc2c(oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c32)c([2H])c1[2H] ; [2H]c1ccc2oc3ccc([2H])c(-c4nc(-c5ccc6ccccc6c5)nc(-c5cc6ccccc6c6ccccc56)n4)c3c2c1]

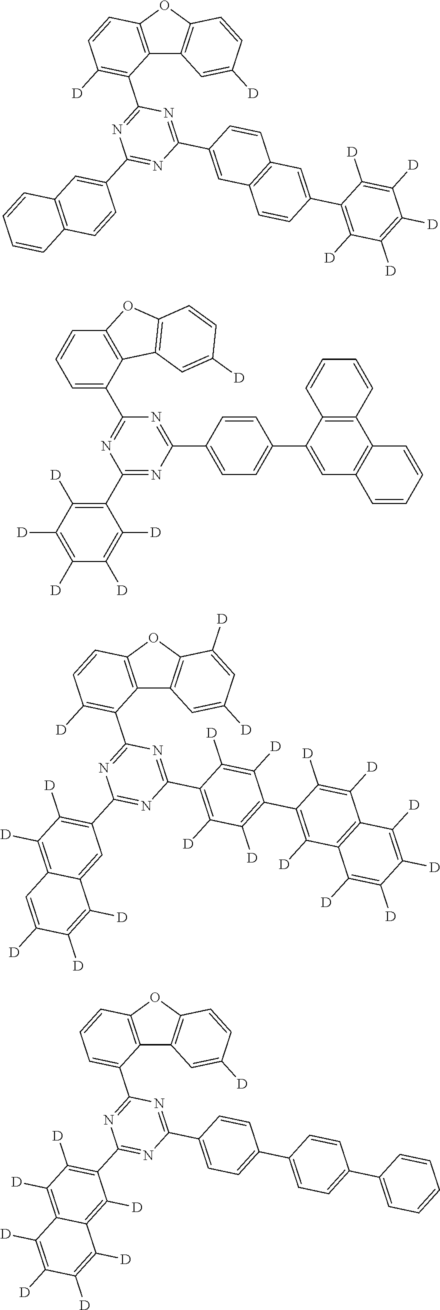

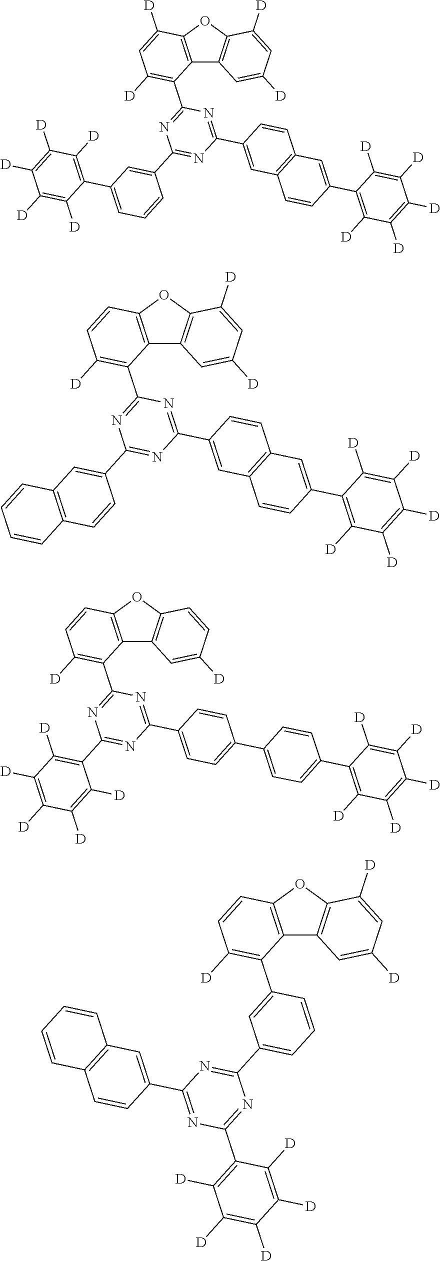

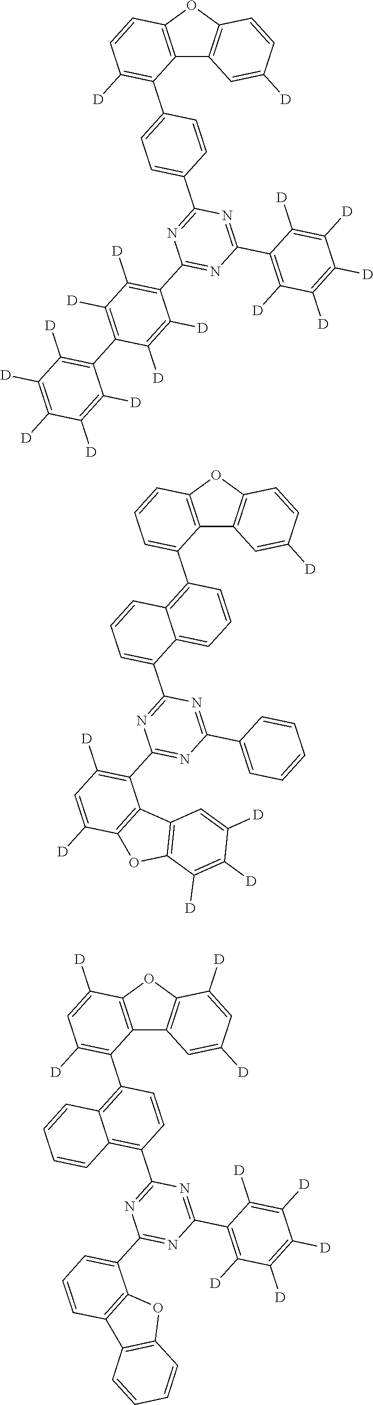 [SMILES: [2H]c1ccc2oc3ccc([2H])c(-c4ccc(-c5nc(-c6c([2H])c([2H])c([2H])c([2H])c6[2H])nc(-c6c([2H])c([2H])c(-c7c([2H])c([2H])c([2H])c([2H])c7[2H])c([2H])c6[2H])n5)cc4)c3c2c1 ; [2H]c1ccc2oc3cccc(-c4cccc5c(-c6nc(-c7ccccc7)nc(-c7c([2H])cc([2H])c8oc9c([2H])c([2H])c([2H])cc9c78)n6)cccc45)c3c2c1 ; [2H]c1cc([2H])c2oc3c([2H])cc([2H])c(-c4ccc(-c5nc(-c6c([2H])c([2H])c([2H])c([2H])c6[2H])nc(-c6cccc7c6oc6ccccc67)n5)c5ccccc45)c3c2c1]

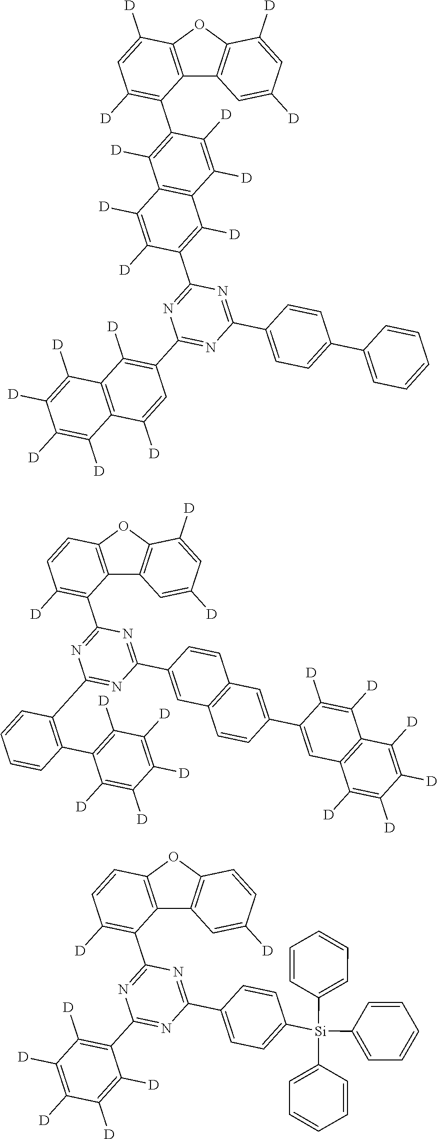

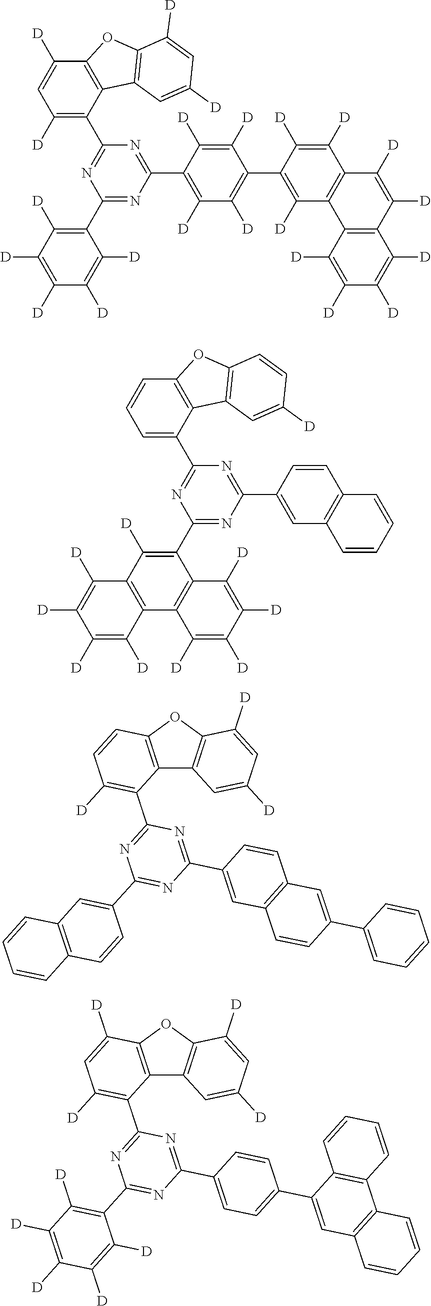

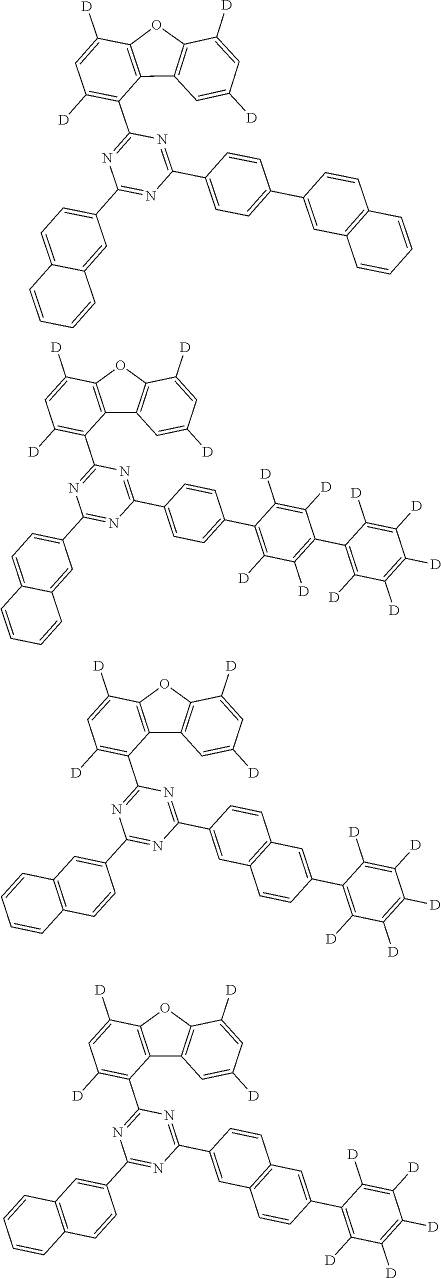

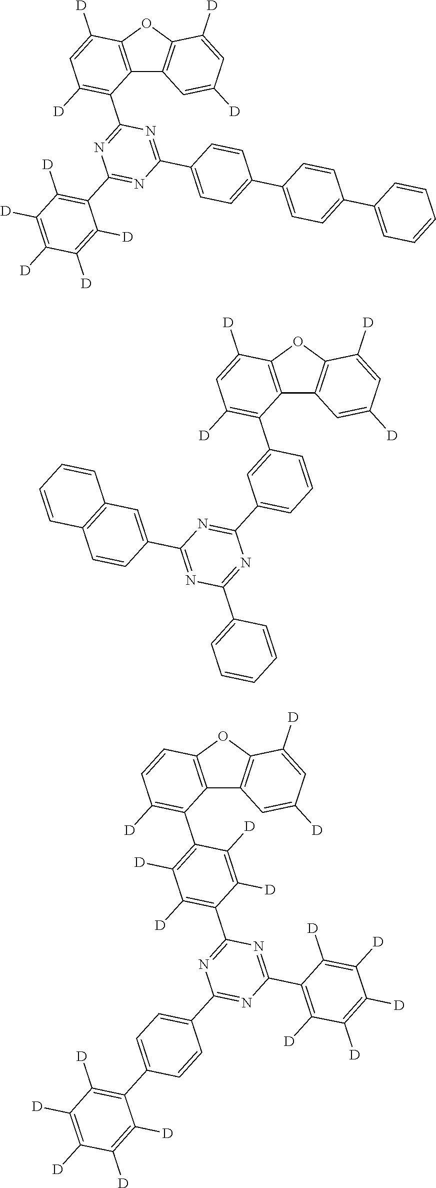 [SMILES: [2H]c1cc([2H])c2oc3c([2H])cc([2H])c(-c4nc(-c5ccc(-c6ccc(-c7ccccc7)cc6)cc5)nc(-c5c([2H])c([2H])c([2H])c([2H])c5[2H])n4)c3c2c1 ; [2H]c1cc([2H])c2oc3c([2H])cc([2H])c(-c4cccc(-c5nc(-c6ccccc6)nc(-c6ccc7ccccc7c6)n5)c4)c3c2c1 ; [2H]c1cc([2H])c2oc3ccc([2H])c(-c4c([2H])c([2H])c(-c5nc(-c6ccc(-c7c([2H])c([2H])c([2H])c([2H])c7[2H])cc6)nc(-c6c([2H])c([2H])c([2H])c([2H])c6[2H])n5)c([2H])c4[2H])c3c2c1]

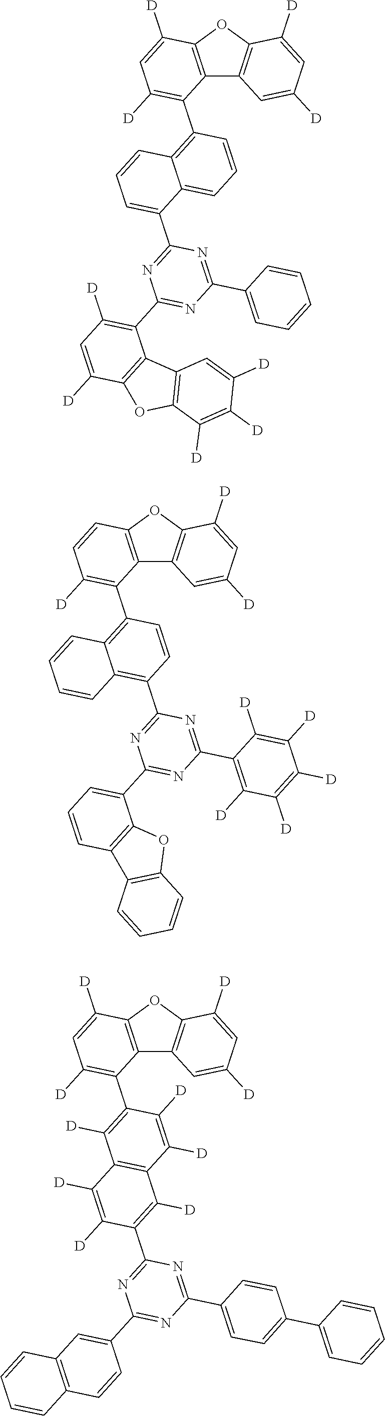

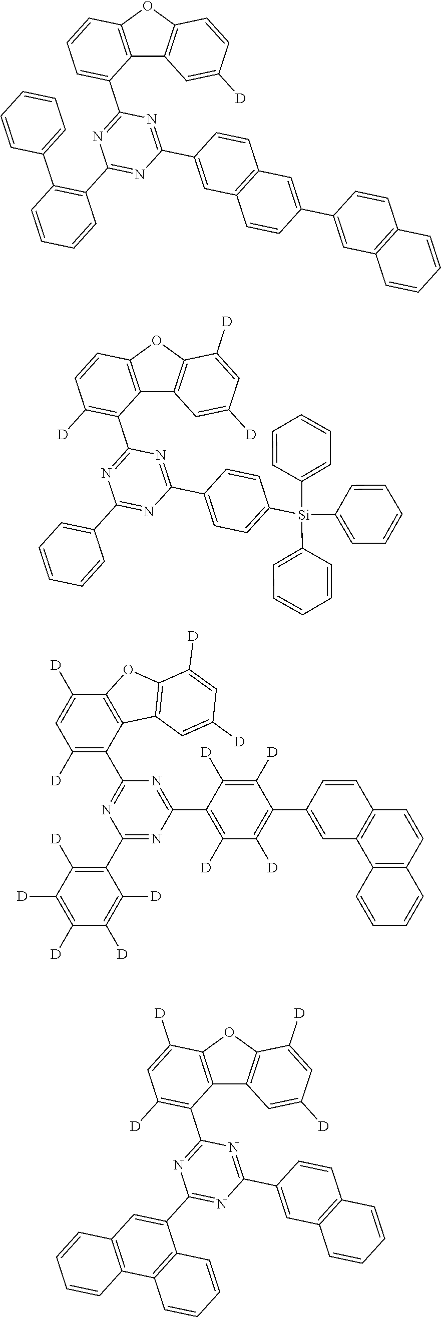 [SMILES: [2H]c1ccc2oc3cccc(-c4nc(-c5ccc6cc(-c7ccc8ccccc8c7)ccc6c5)nc(-c5ccccc5-c5ccccc5)n4)c3c2c1 ; [2H]c1cc([2H])c2oc3ccc([2H])c(-c4nc(-c5ccccc5)nc(-c5ccc([Si](c6ccccc6)(c6ccccc6)c6ccccc6)cc5)n4)c3c2c1 ; [2H]c1cc([2H])c2oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c3c2c1 ; [2H]c1cc([2H])c2oc3c([2H])cc([2H])c(-c4nc(-c5ccc6ccccc6c5)nc(-c5cc6ccccc6c6ccccc56)n4)c3c2c1]

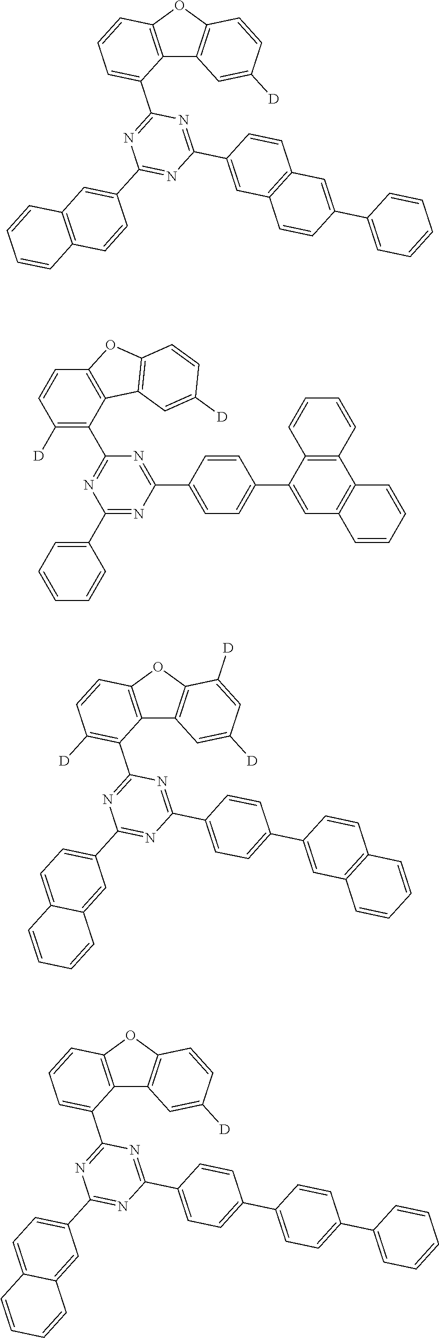 [SMILES: [2H]c1ccc2oc3cccc(-c4nc(-c5ccc6ccccc6c5)nc(-c5ccc6cc(-c7ccccc7)ccc6c5)n4)c3c2c1 ; [2H]c1ccc2oc3ccc([2H])c(-c4nc(-c5ccccc5)nc(-c5ccc(-c6cc7ccccc7c7ccccc67)cc5)n4)c3c2c1 ; [2H]c1cc([2H])c2oc3ccc([2H])c(-c4nc(-c5ccc(-c6ccc7ccccc7c6)cc5)nc(-c5ccc6ccccc6c5)n4)c3c2c1 ; [2H]c1ccc2oc3cccc(-c4nc(-c5ccc(-c6ccc(-c7ccccc7)cc6)cc5)nc(-c5ccc6ccccc6c5)n4)c3c2c1]

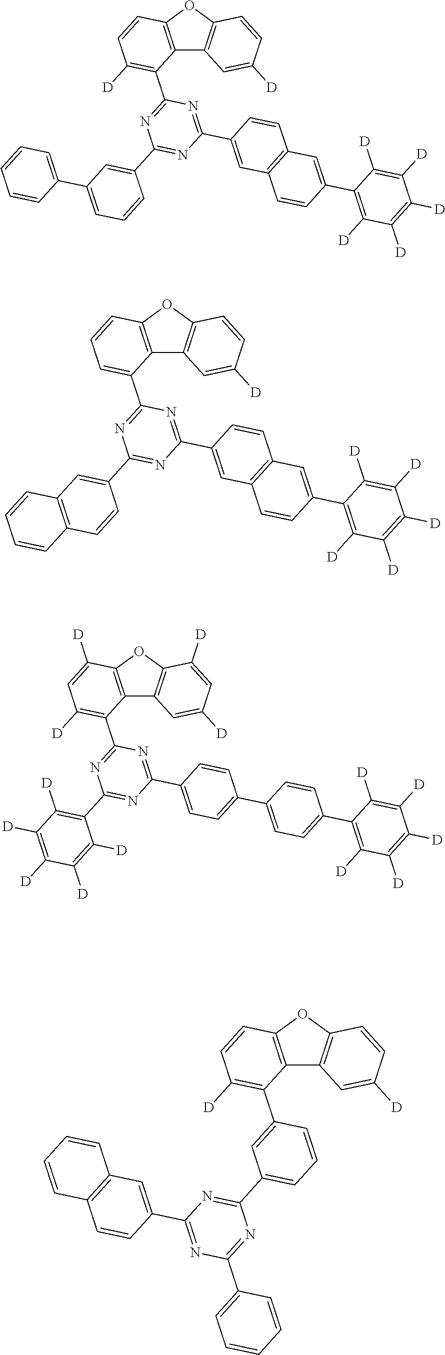 [SMILES: [2H]c1ccc2oc3ccc([2H])c(-c4nc(-c5cccc(-c6ccccc6)c5)nc(-c5ccc6cc(-c7c([2H])c([2H])c([2H])c([2H])c7[2H])ccc6c5)n4)c3c2c1 ; [2H]c1ccc2oc3cccc(-c4nc(-c5ccc6ccccc6c5)nc(-c5ccc6cc(-c7c([2H])c([2H])c([2H])c([2H])c7[2H])ccc6c5)n4)c3c2c1 ; [2H]c1cc([2H])c2oc3c([2H])cc([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c3c2c1 ; [2H]c1ccc2oc3ccc([2H])c(-c4cccc(-c5nc(-c6ccccc6)nc(-c6ccc7ccccc7c6)n5)c4)c3c2c1]

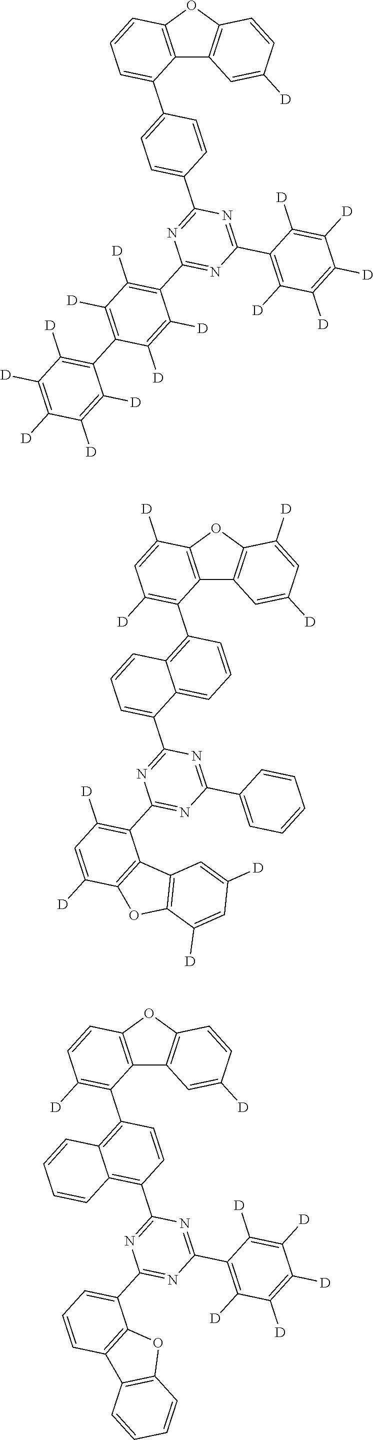 [SMILES: [2H]c1ccc2oc3cccc(-c4ccc(-c5nc(-c6c([2H])c([2H])c([2H])c([2H])c6[2H])nc(-c6c([2H])c([2H])c(-c7c([2H])c([2H])c([2H])c([2H])c7[2H])c([2H])c6[2H])n5)cc4)c3c2c1 ; [2H]c1cc([2H])c2oc3c([2H])cc([2H])c(-c4nc(-c5ccccc5)nc(-c5cccc6c(-c7c([2H])cc([2H])c8oc9c([2H])cc([2H])cc9c78)cccc56)n4)c3c2c1 ; [2H]c1ccc2oc3ccc([2H])c(-c4ccc(-c5nc(-c6c([2H])c([2H])c([2H])c([2H])c6[2H])nc(-c6cccc7c6oc6ccccc67)n5)c5ccccc45)c3c2c1]

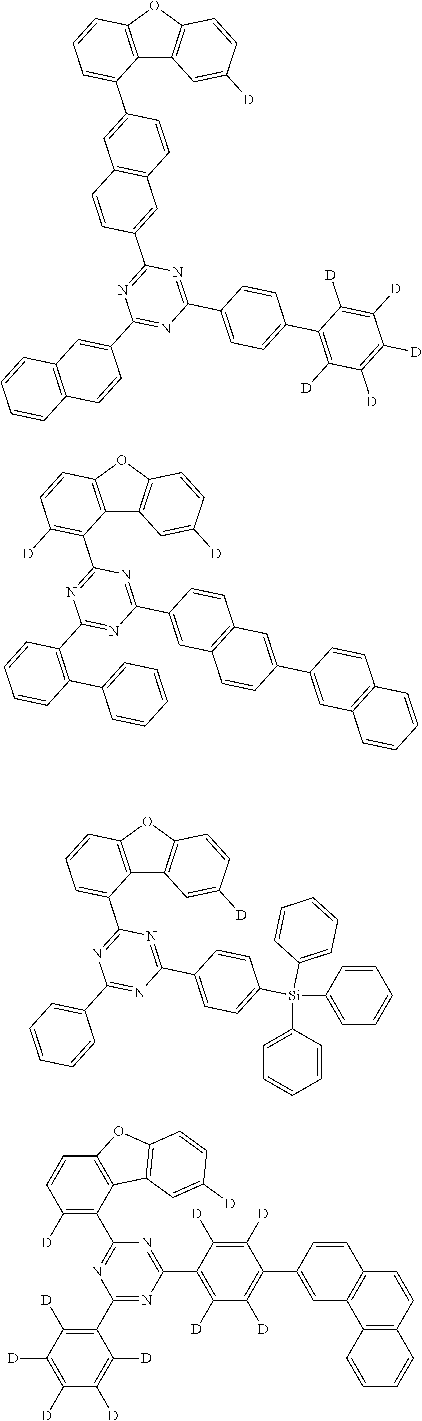 [SMILES: [2H]c1ccc2oc3cccc(-c4ccc5cc(-c6nc(-c7ccc(-c8c([2H])c([2H])c([2H])c([2H])c8[2H])cc7)nc(-c7ccc8ccccc8c7)n6)ccc5c4)c3c2c1 ; [2H]c1ccc2oc3ccc([2H])c(-c4nc(-c5ccc6cc(-c7ccc8ccccc8c7)ccc6c5)nc(-c5ccccc5-c5ccccc5)n4)c3c2c1 ; [2H]c1ccc2oc3cccc(-c4nc(-c5ccccc5)nc(-c5ccc([Si](c6ccccc6)(c6ccccc6)c6ccccc6)cc5)n4)c3c2c1 ; [2H]c1ccc2oc3ccc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c3c2c1]

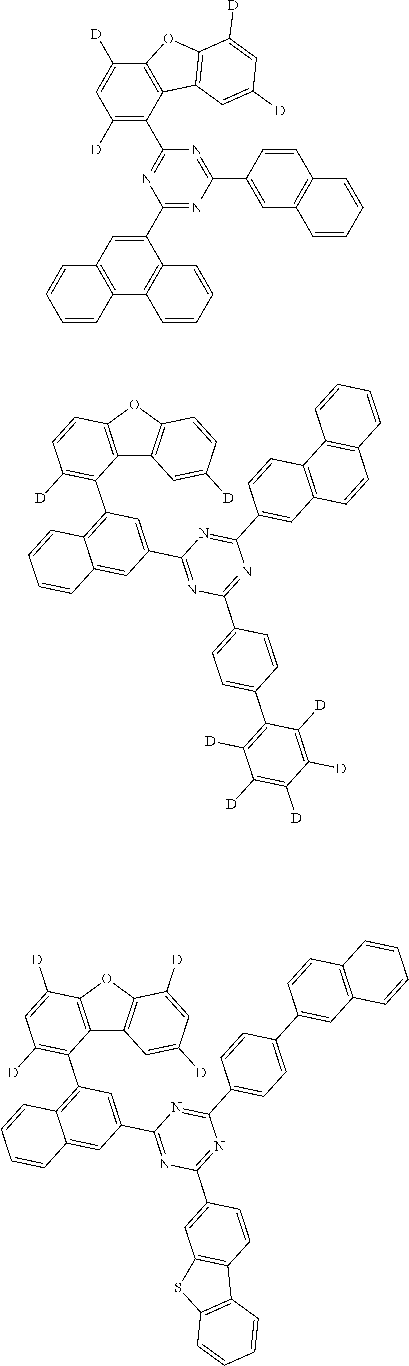 [SMILES: [2H]c1cc([2H])c2oc3c([2H])cc([2H])c(-c4nc(-c5ccc6ccccc6c5)nc(-c5cc6ccccc6c6ccccc56)n4)c3c2c1 ; [2H]c1ccc2oc3ccc([2H])c(-c4cc(-c5nc(-c6ccc(-c7c([2H])c([2H])c([2H])c([2H])c7[2H])cc6)nc(-c6ccc7c(ccc8ccccc87)c6)n5)cc5ccccc45)c3c2c1 ; [2H]c1cc([2H])c2oc3c([2H])cc([2H])c(-c4cc(-c5nc(-c6ccc(-c7ccc8ccccc8c7)cc6)nc(-c6ccc7c(c6)sc6ccccc67)n5)cc5ccccc45)c3c2c1]

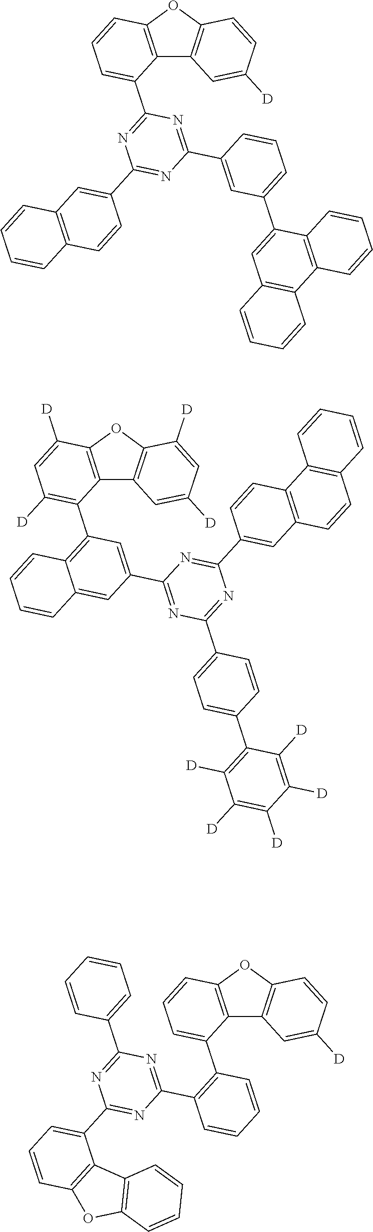 [SMILES: [2H]c1ccc2oc3cccc(-c4nc(-c5cccc(-c6cc7ccccc7c7ccccc67)c5)nc(-c5ccc6ccccc6c5)n4)c3c2c1 ; [2H]c1cc([2H])c2oc3c([2H])cc([2H])c(-c4cc(-c5nc(-c6ccc(-c7c([2H])c([2H])c([2H])c([2H])c7[2H])cc6)nc(-c6ccc7c(ccc8ccccc87)c6)n5)cc5ccccc45)c3c2c1 ; [2H]c1ccc2oc3cccc(-c4ccccc4-c4nc(-c5ccccc5)nc(-c5cccc6oc7ccccc7c56)n4)c3c2c1]

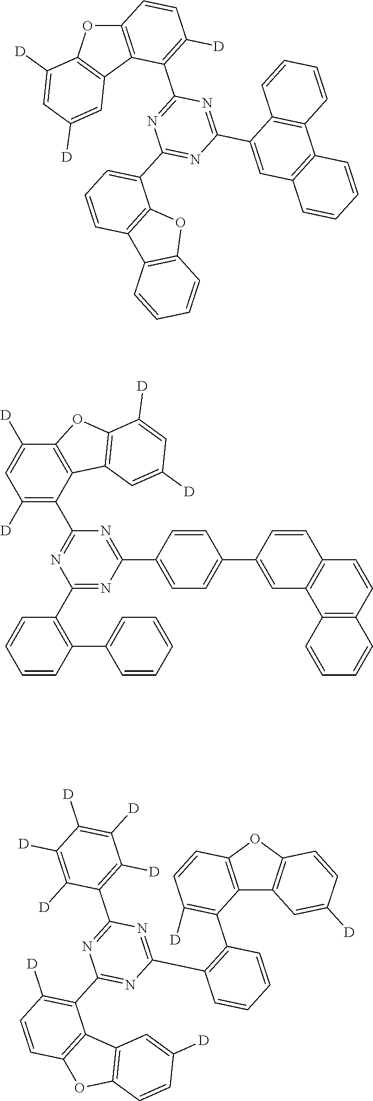 [SMILES: [2H]c1cc([2H])c2oc3ccc([2H])c(-c4nc(-c5cc6ccccc6c6ccccc56)nc(-c5cccc6c5oc5ccccc56)n4)c3c2c1 ; [2H]c1cc([2H])c2oc3c([2H])cc([2H])c(-c4nc(-c5ccc(-c6ccc7ccc8ccccc8c7c6)cc5)nc(-c5ccccc5-c5ccccc5)n4)c3c2c1 ; [2H]c1ccc2oc3ccc([2H])c(-c4nc(-c5ccccc5-c5c([2H])ccc6oc7ccc([2H])cc7c56)nc(-c5c([2H])c([2H])c([2H])c([2H])c5[2H])n4)c3c2c1]

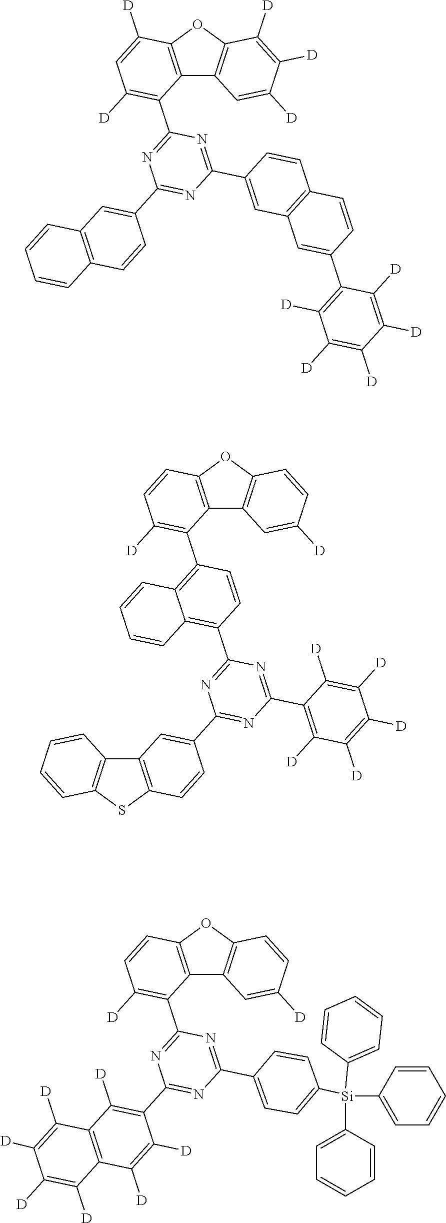 [SMILES: [2H]c1cc2c(oc3c([2H])cc([2H])c(-c4nc(-c5ccc6ccccc6c5)nc(-c5ccc6ccc(-c7c([2H])c([2H])c([2H])c([2H])c7[2H])cc6c5)n4)c32)c([2H])c1[2H] ; [2H]c1ccc2oc3ccc([2H])c(-c4ccc(-c5nc(-c6ccc7sc8ccccc8c7c6)nc(-c6c([2H])c([2H])c([2H])c([2H])c6[2H])n5)c5ccccc45)c3c2c1 ; [2H]c1ccc2oc3ccc([2H])c(-c4nc(-c5ccc([Si](c6ccccc6)(c6ccccc6)c6ccccc6)cc5)nc(-c5c([2H])c([2H])c6c([2H])c([2H])c([2H])c([2H])c6c5[2H])n4)c3c2c1]

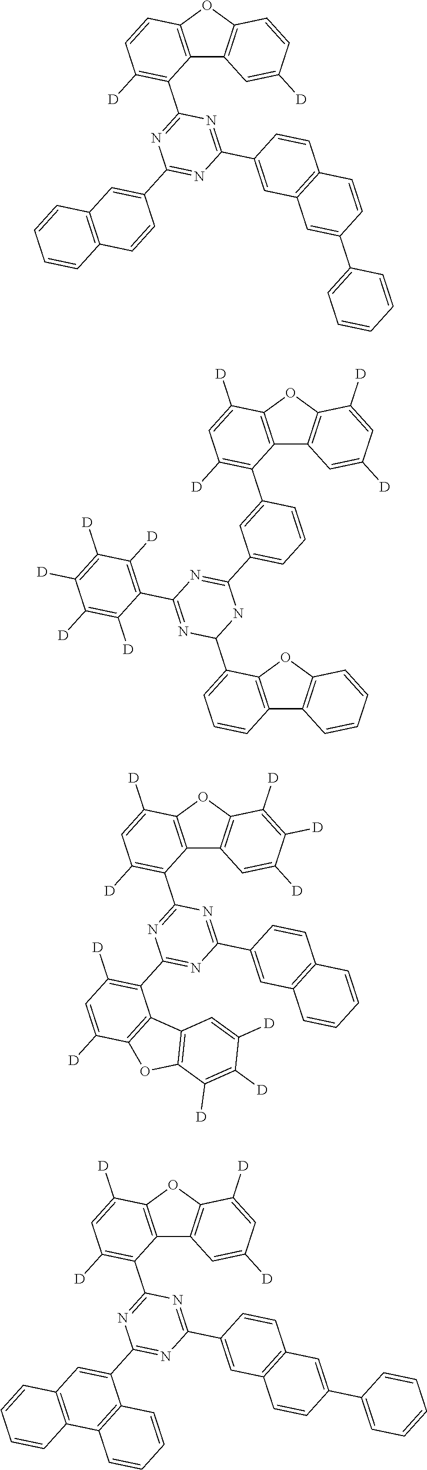

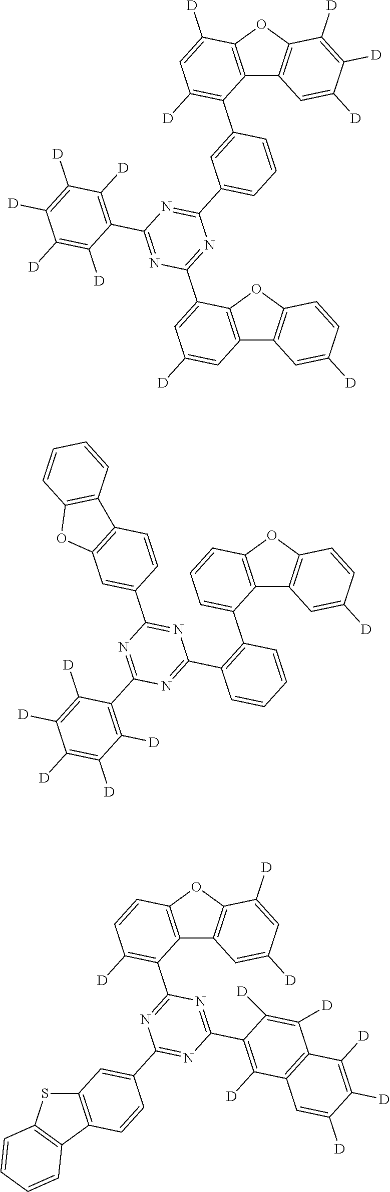 [SMILES: [2H]c1ccc2oc3c(-c4nc(-c5cccc(-c6c([2H])cc([2H])c7oc8c([2H])c([2H])c([2H])cc8c67)c5)nc(-c5c([2H])c([2H])c([2H])c([2H])c5[2H])n4)cc([2H])cc3c2c1 ; [2H]c1ccc2oc3cccc(-c4ccccc4-c4nc(-c5ccc6c(c5)oc5ccccc56)nc(-c5c([2H])c([2H])c([2H])c([2H])c5[2H])n4)c3c2c1 ; [2H]c1cc([2H])c2oc3ccc([2H])c(-c4nc(-c5ccc6c(c5)sc5ccccc56)nc(-c5c([2H])c([2H])c6c([2H])c([2H])c([2H])cc6c5[2H])n4)c3c2c1]

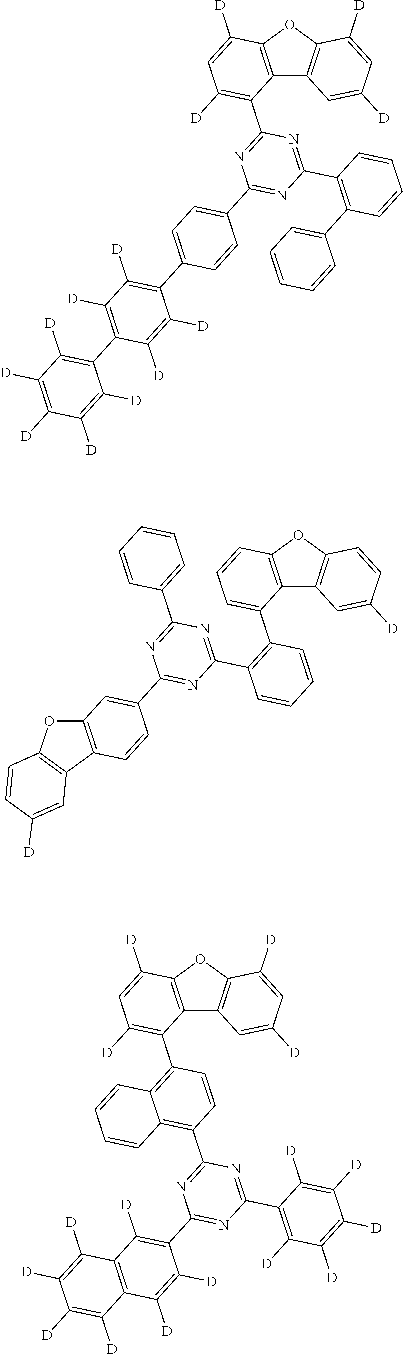 [SMILES: [2H]c1cc([2H])c2oc3c([2H])cc([2H])c(-c4nc(-c5ccc(-c6c([2H])c([2H])c(-c7c([2H])c([2H])c([2H])c([2H])c7[2H])c([2H])c6[2H])cc5)nc(-c5ccccc5-c5ccccc5)n4)c3c2c1 ; [2H]c1ccc2oc3cc(-c4nc(-c5ccccc5)nc(-c5ccccc5-c5cccc6oc7ccc([2H])cc7c56)n4)ccc3c2c1 ; [2H]c1cc([2H])c2oc3c([2H])cc([2H])c(-c4ccc(-c5nc(-c6c([2H])c([2H])c([2H])c([2H])c6[2H])nc(-c6c([2H])c([2H])c7c([2H])c([2H])c([2H])c([2H])c7c6[2H])n5)c5ccccc45)c3c2c1]

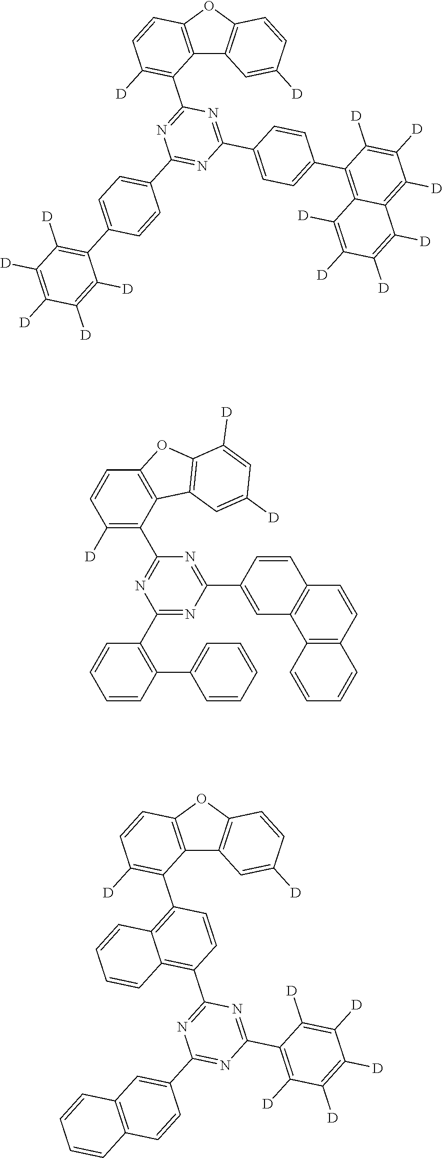 [SMILES: [2H]c1ccc2oc3ccc([2H])c(-c4nc(-c5ccc(-c6c([2H])c([2H])c([2H])c([2H])c6[2H])cc5)nc(-c5ccc(-c6c([2H])c([2H])c([2H])c7c([2H])c([2H])c([2H])c([2H])c67)cc5)n4)c3c2c1 ; [2H]c1cc([2H])c2oc3ccc([2H])c(-c4nc(-c5ccc6ccc7ccccc7c6c5)nc(-c5ccccc5-c5ccccc5)n4)c3c2c1 ; [2H]c1ccc2oc3ccc([2H])c(-c4ccc(-c5nc(-c6ccc7ccccc7c6)nc(-c6c([2H])c([2H])c([2H])c([2H])c6[2H])n5)c5ccccc45)c3c2c1]

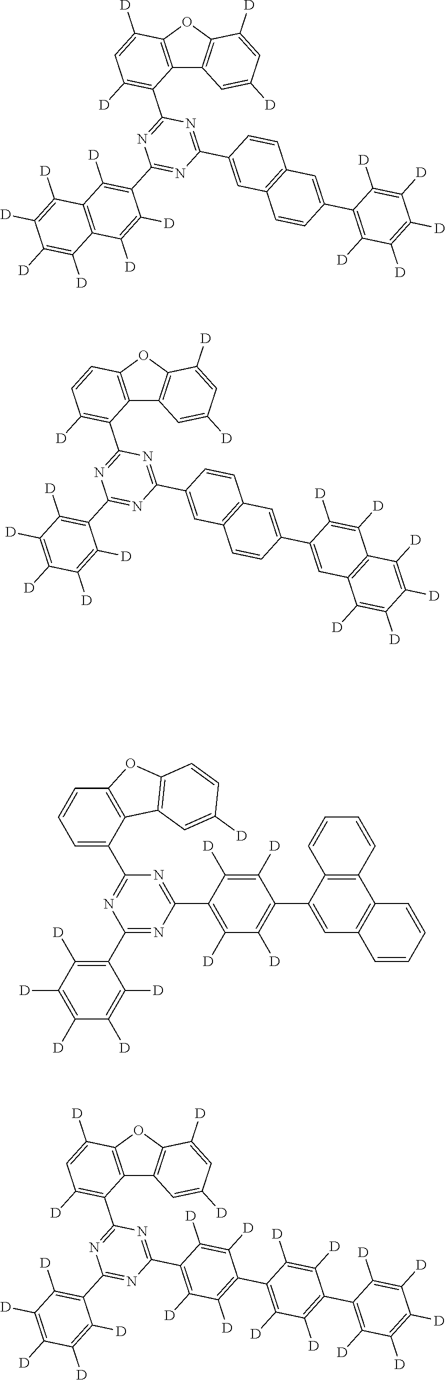

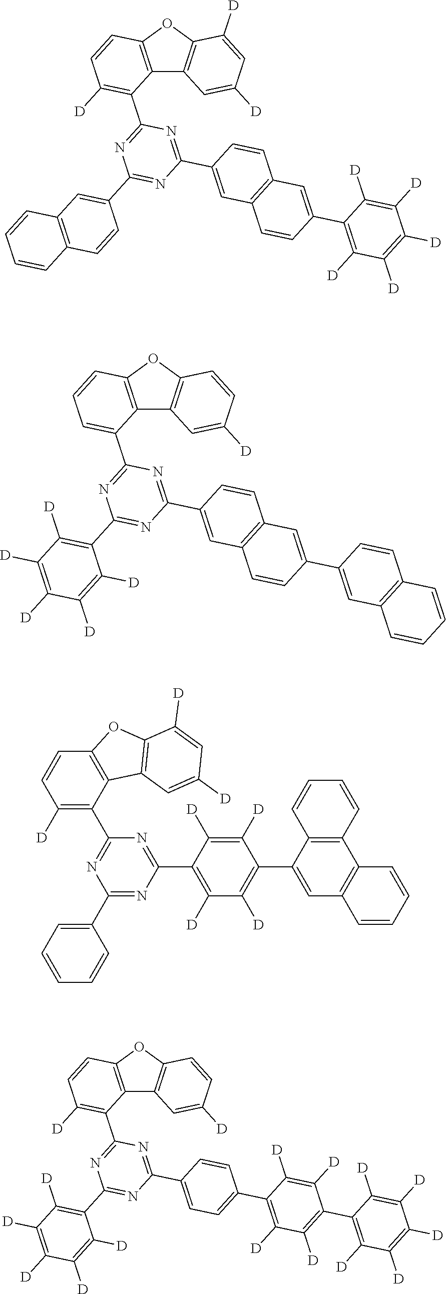

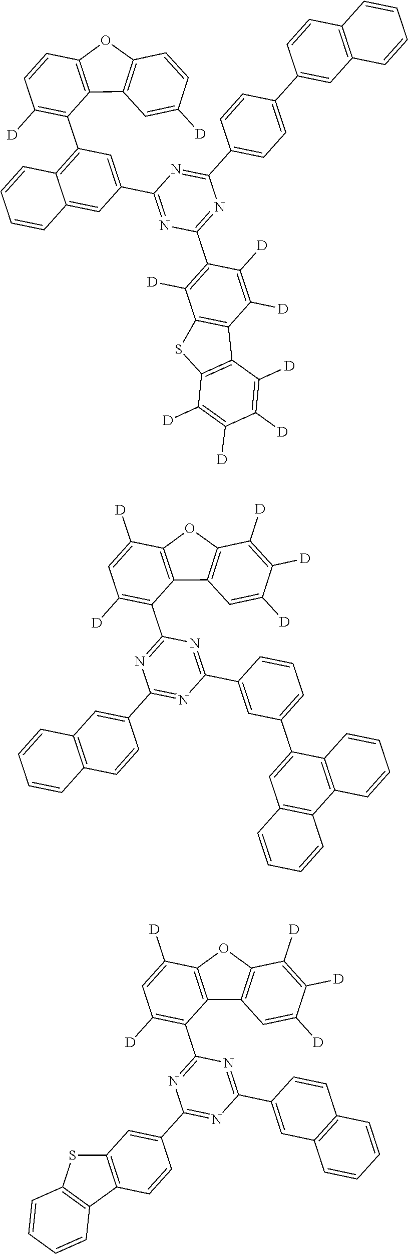 [SMILES: [2H]c1ccc2oc3ccc([2H])c(-c4cc(-c5nc(-c6ccc(-c7ccc8ccccc8c7)cc6)nc(-c6c([2H])c([2H])c7c(sc8c([2H])c([2H])c([2H])c([2H])c87)c6[2H])n5)cc5ccccc45)c3c2c1 ; [2H]c1cc2c(oc3c([2H])cc([2H])c(-c4nc(-c5cccc(-c6cc7ccccc7c7ccccc67)c5)nc(-c5ccc6ccccc6c5)n4)c32)c([2H])c1[2H] ; [2H]c1cc2c(oc3c([2H])cc([2H])c(-c4nc(-c5ccc6ccccc6c5)nc(-c5ccc6c(c5)sc5ccccc56)n4)c32)c([2H])c1[2H]]

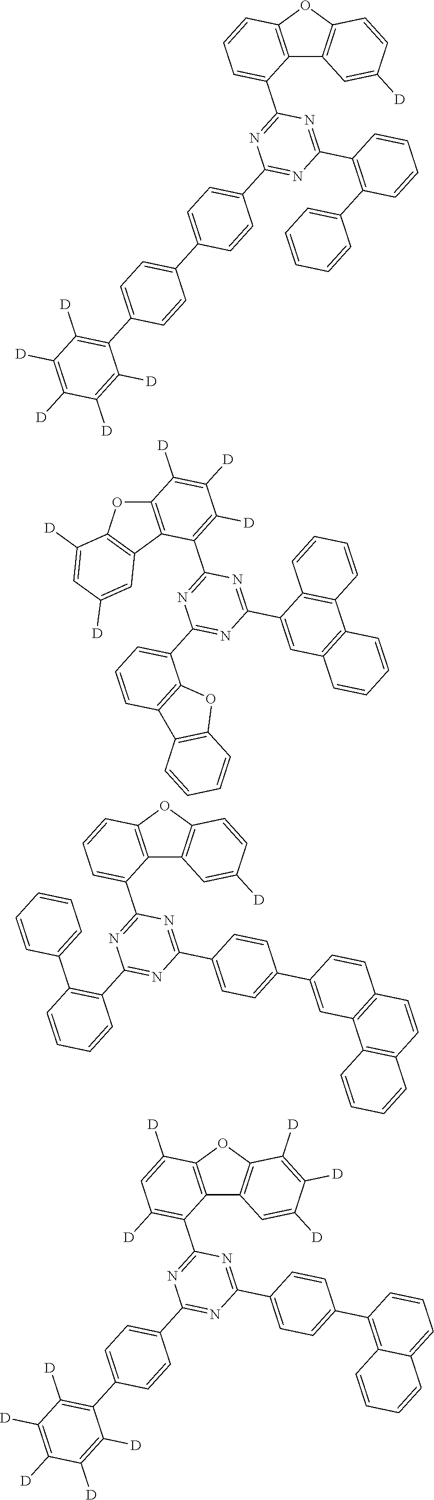

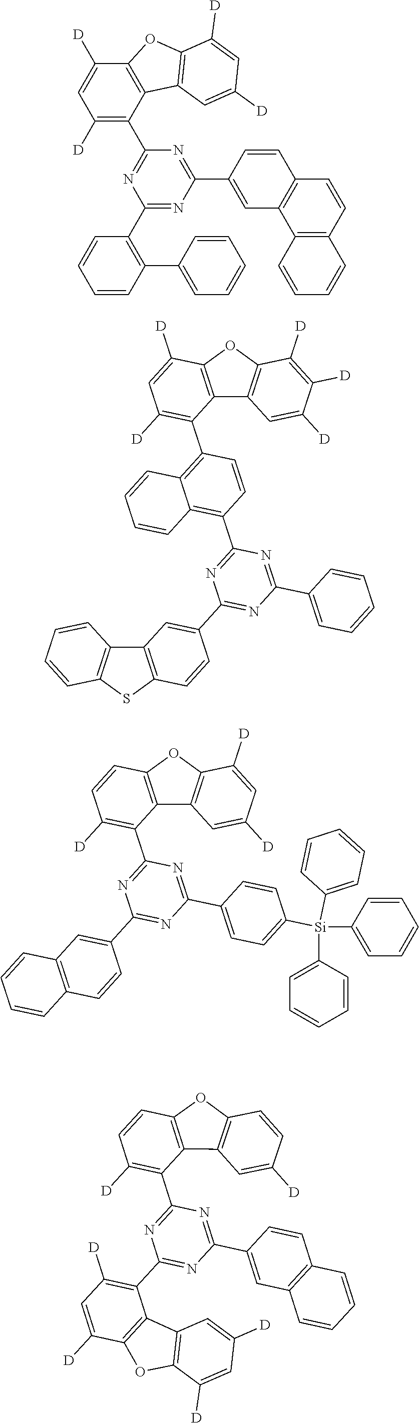

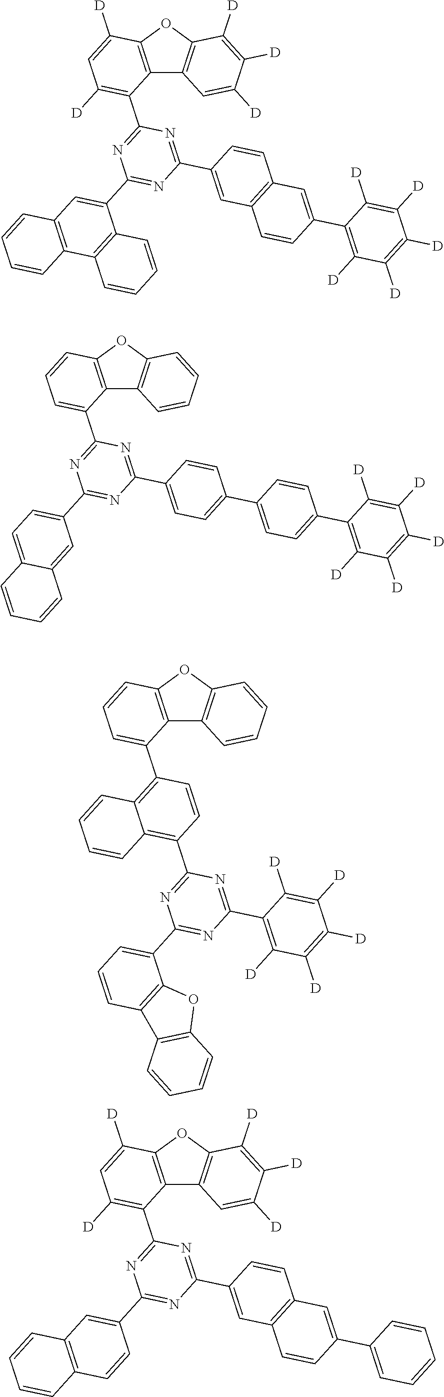

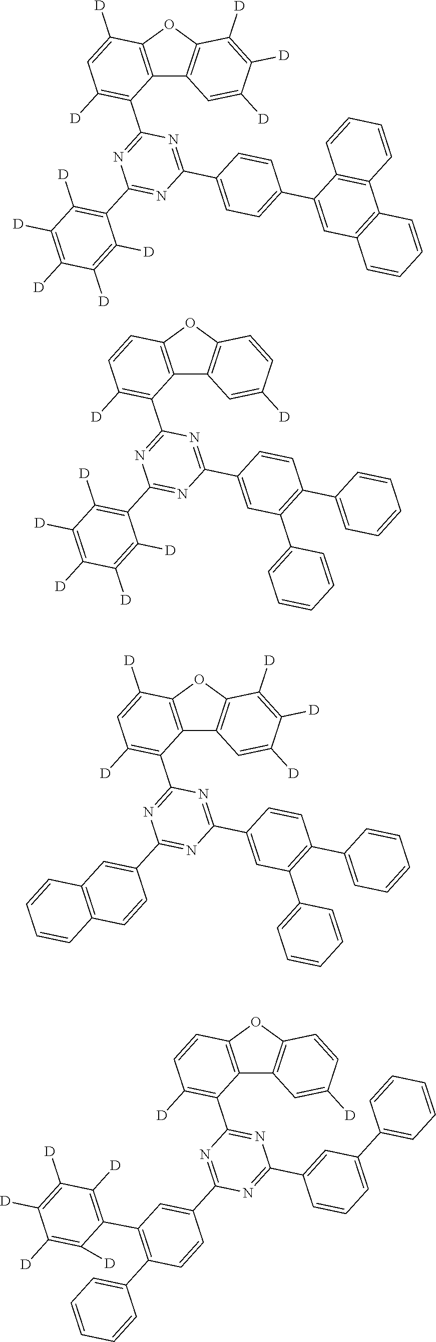

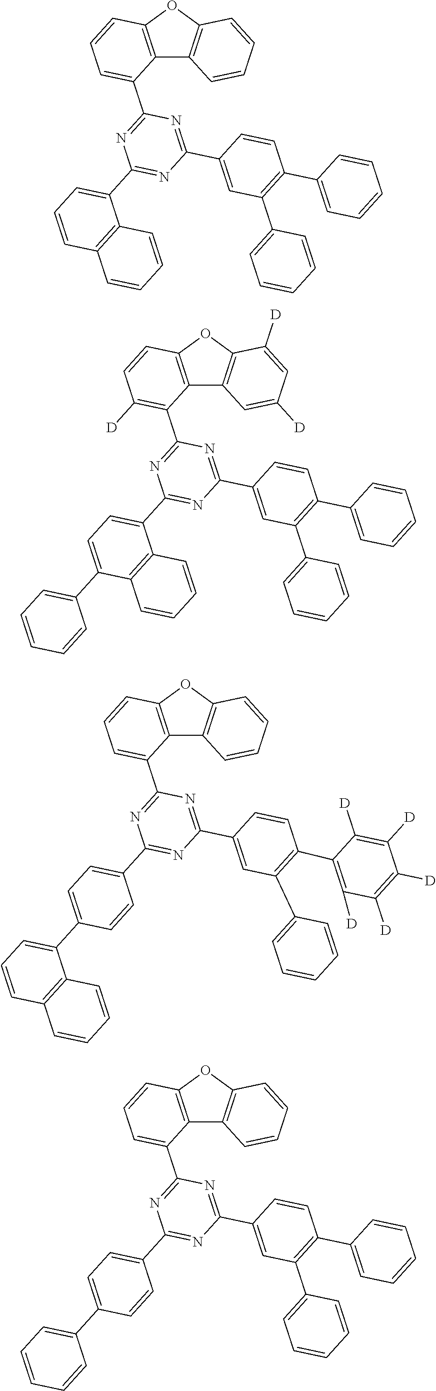

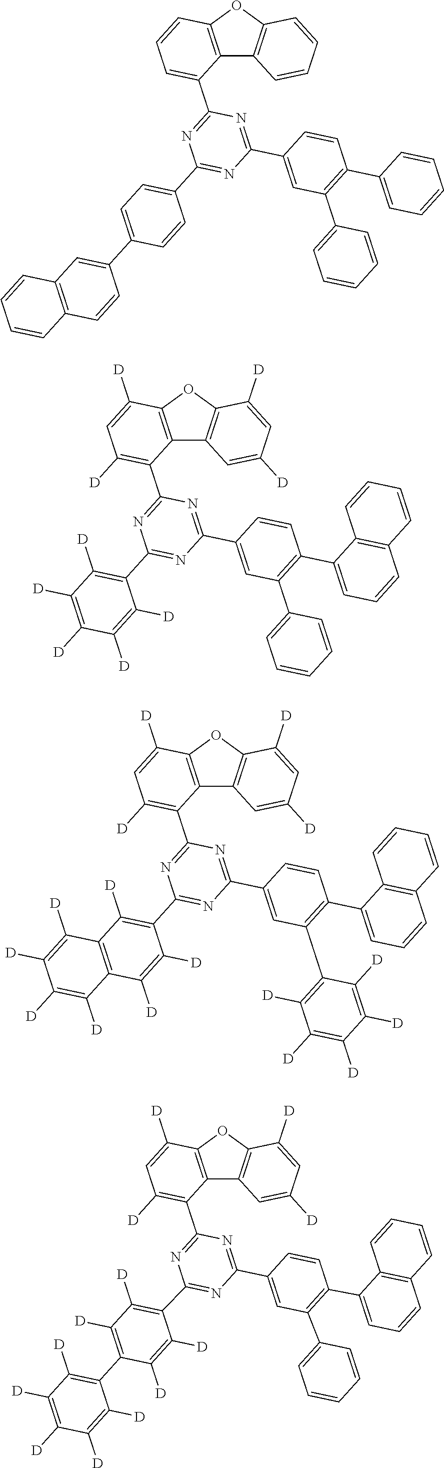

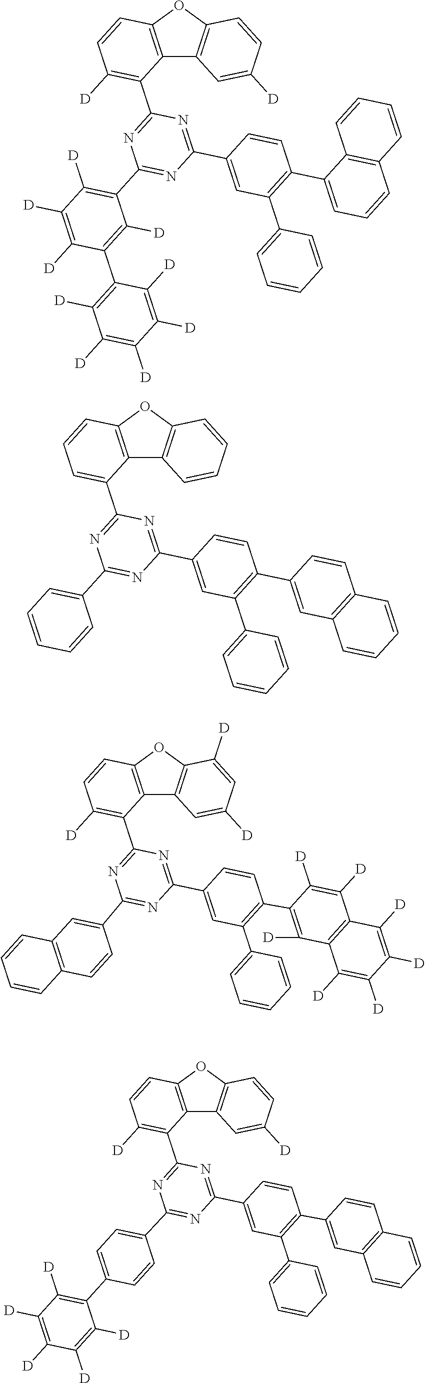

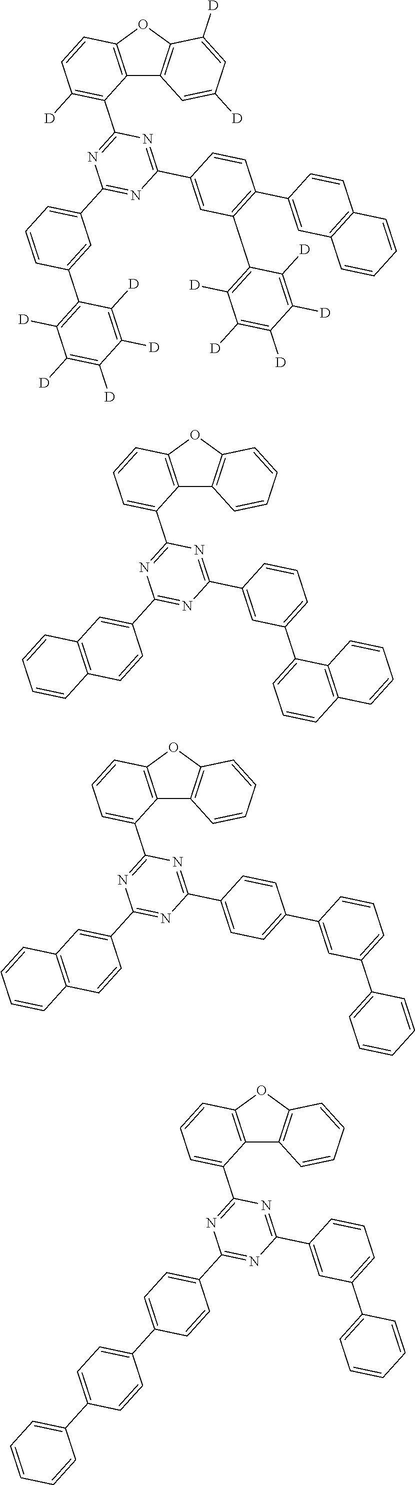 [SMILES: [2H]c1cc([2H])c2oc3ccc([2H])c(-c4nc(-c5cccc(-c6c([2H])c([2H])c([2H])c([2H])c6[2H])c5)nc(-c5ccc(-c6ccc7ccccc7c6)c(-c6c([2H])c([2H])c([2H])c([2H])c6[2H])c5)n4)c3c2c1 ; c1cc(-c2nc(-c3ccc4ccccc4c3)nc(-c3cccc4oc5ccccc5c34)n2)cc(-c2cccc3ccccc23)c1 ; c1ccc(-c2cccc(-c3ccc(-c4nc(-c5ccc6ccccc6c5)nc(-c5cccc6oc7ccccc7c56)n4)cc3)c2)cc1 ; c1ccc(-c2ccc(-c3ccc(-c4nc(-c5cccc(-c6ccccc6)c5)nc(-c5cccc6oc7ccccc7c56)n4)cc3)cc2)cc1]

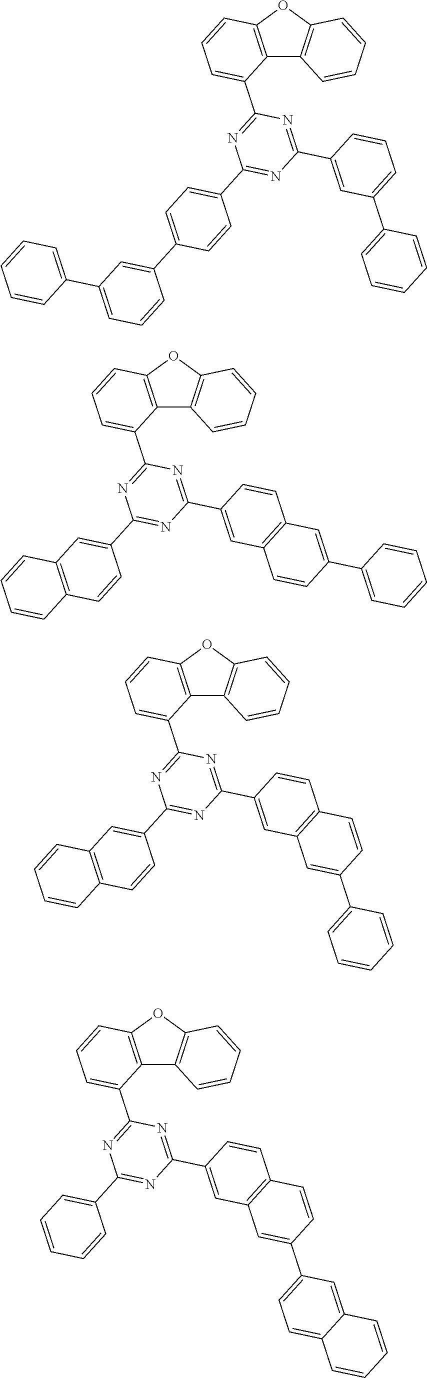

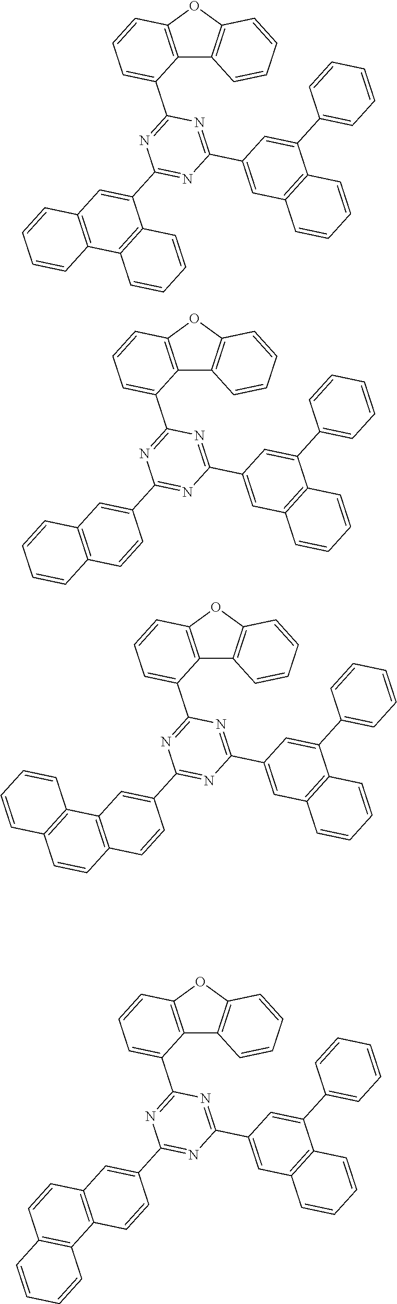

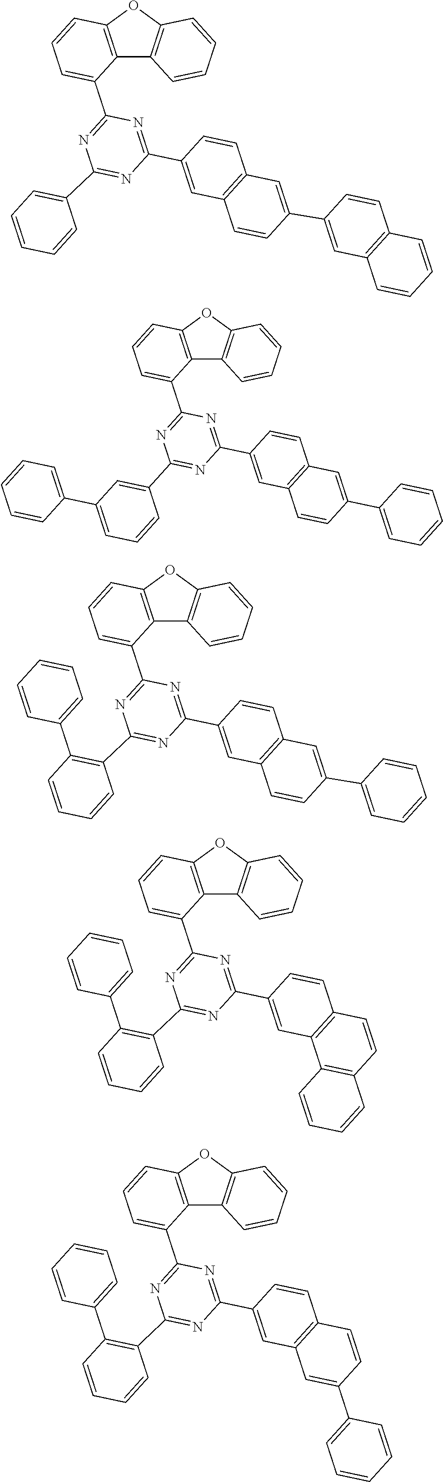

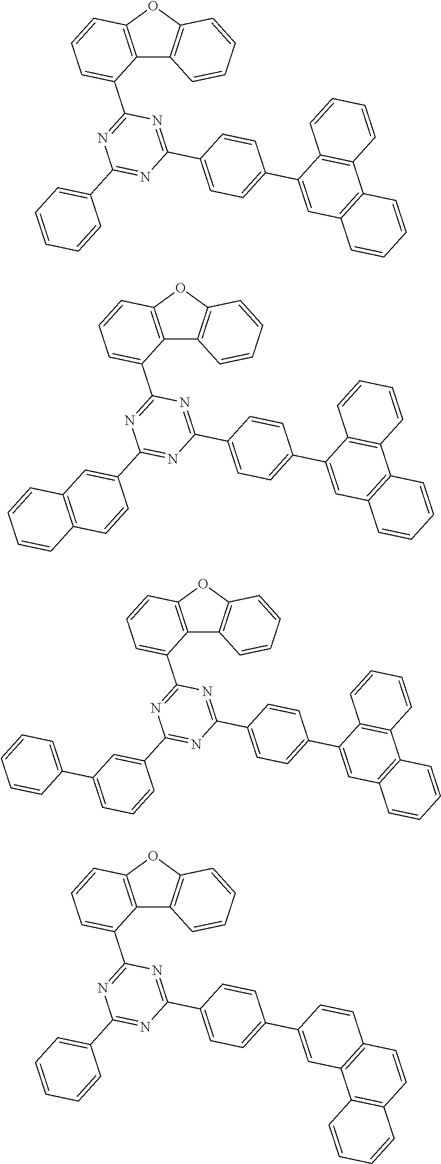

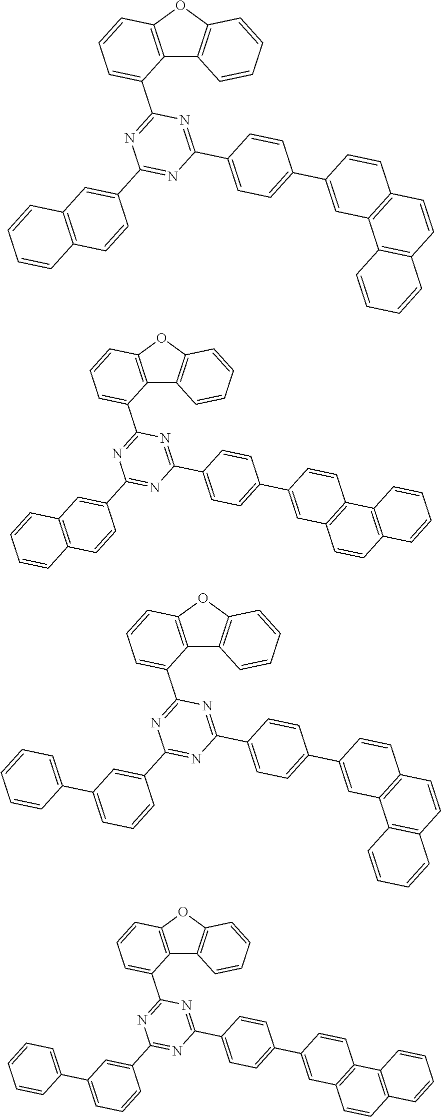 [SMILES: c1ccc2cc(-c3nc(-c4ccc(-c5ccc6ccc7ccccc7c6c5)cc4)nc(-c4cccc5oc6ccccc6c45)n3)ccc2c1 ; c1ccc2cc(-c3nc(-c4ccc(-c5ccc6c(ccc7ccccc76)c5)cc4)nc(-c4cccc5oc6ccccc6c45)n3)ccc2c1 ; c1ccc(-c2cccc(-c3nc(-c4ccc(-c5ccc6ccc7ccccc7c6c5)cc4)nc(-c4cccc5oc6ccccc6c45)n3)c2)cc1 ; c1ccc(-c2cccc(-c3nc(-c4ccc(-c5ccc6c(ccc7ccccc76)c5)cc4)nc(-c4cccc5oc6ccccc6c45)n3)c2)cc1]

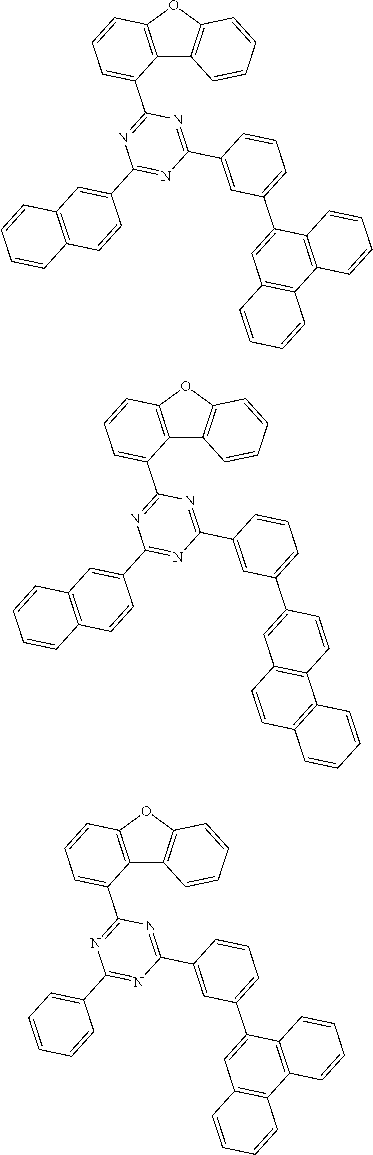 [SMILES: c1cc(-c2nc(-c3ccc4ccccc4c3)nc(-c3cccc4oc5ccccc5c34)n2)cc(-c2cc3ccccc3c3ccccc23)c1 ; c1cc(-c2ccc3c(ccc4ccccc43)c2)cc(-c2nc(-c3ccc4ccccc4c3)nc(-c3cccc4oc5ccccc5c34)n2)c1 ; c1ccc(-c2nc(-c3cccc(-c4cc5ccccc5c5ccccc45)c3)nc(-c3cccc4oc5ccccc5c34)n2)cc1]

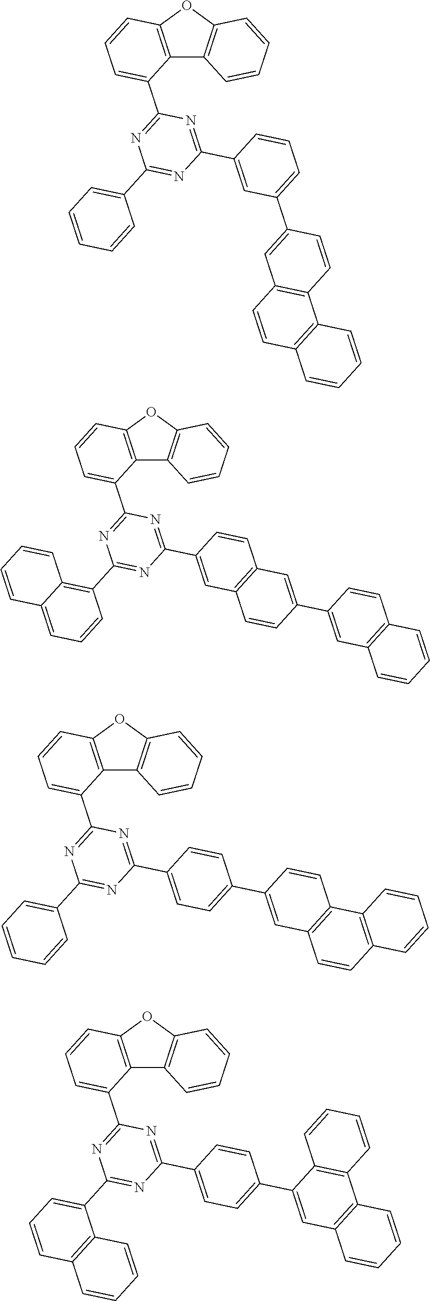 [SMILES: c1ccc(-c2nc(-c3cccc(-c4ccc5c(ccc6ccccc65)c4)c3)nc(-c3cccc4oc5ccccc5c34)n2)cc1 ; c1ccc2cc(-c3ccc4cc(-c5nc(-c6cccc7ccccc67)nc(-c6cccc7oc8ccccc8c67)n5)ccc4c3)ccc2c1 ; c1ccc(-c2nc(-c3ccc(-c4ccc5c(ccc6ccccc65)c4)cc3)nc(-c3cccc4oc5ccccc5c34)n2)cc1 ; c1ccc2c(-c3nc(-c4ccc(-c5cc6ccccc6c6ccccc56)cc4)nc(-c4cccc5oc6ccccc6c45)n3)cccc2c1]

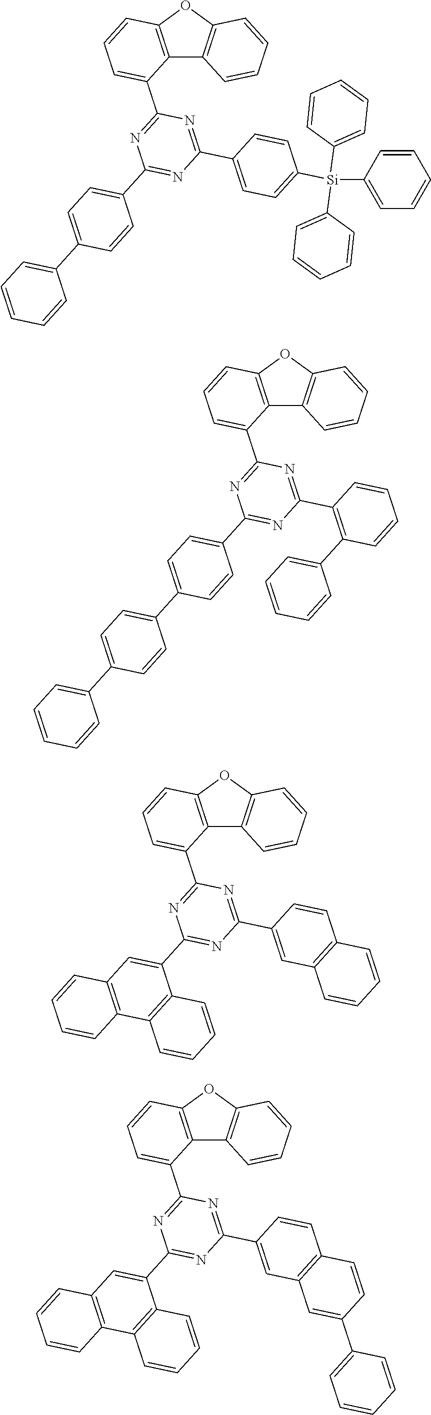

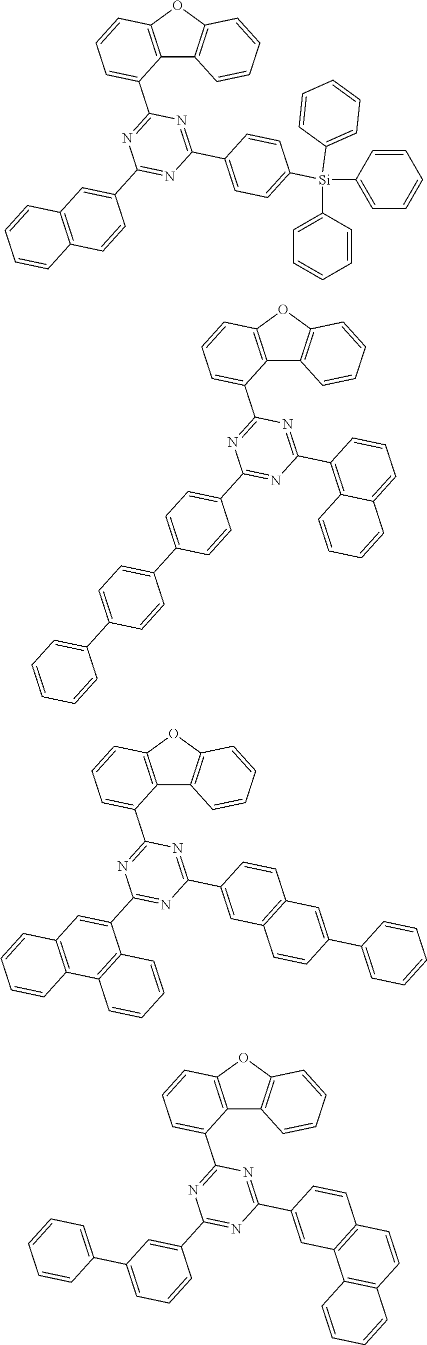 [SMILES: c1ccc([Si](c2ccccc2)(c2ccccc2)c2ccc(-c3nc(-c4ccc5ccccc5c4)nc(-c4cccc5oc6ccccc6c45)n3)cc2)cc1 ; c1ccc(-c2ccc(-c3ccc(-c4nc(-c5cccc6ccccc56)nc(-c5cccc6oc7ccccc7c56)n4)cc3)cc2)cc1 ; c1ccc(-c2ccc3cc(-c4nc(-c5cc6ccccc6c6ccccc56)nc(-c5cccc6oc7ccccc7c56)n4)ccc3c2)cc1 ; c1ccc(-c2cccc(-c3nc(-c4ccc5ccc6ccccc6c5c4)nc(-c4cccc5oc6ccccc6c45)n3)c2)cc1]

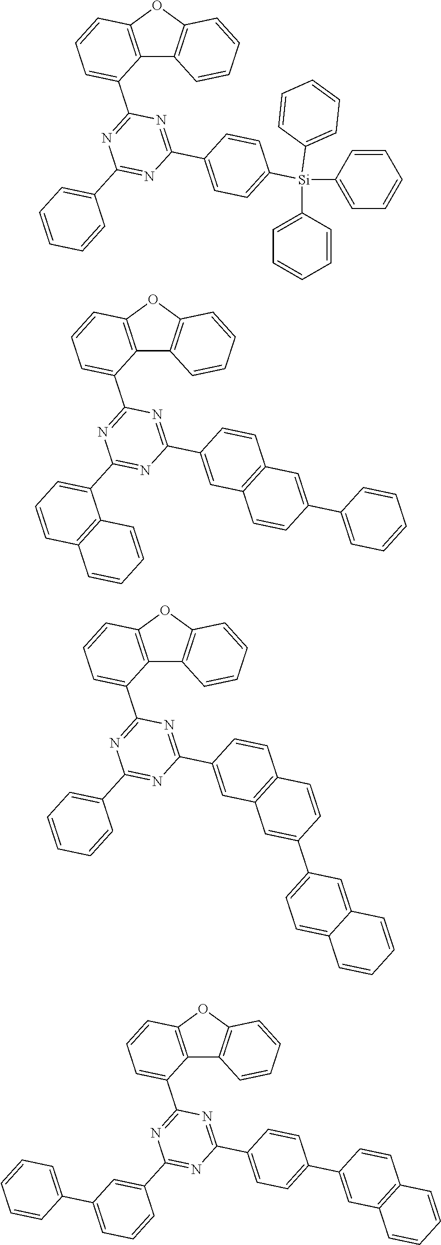

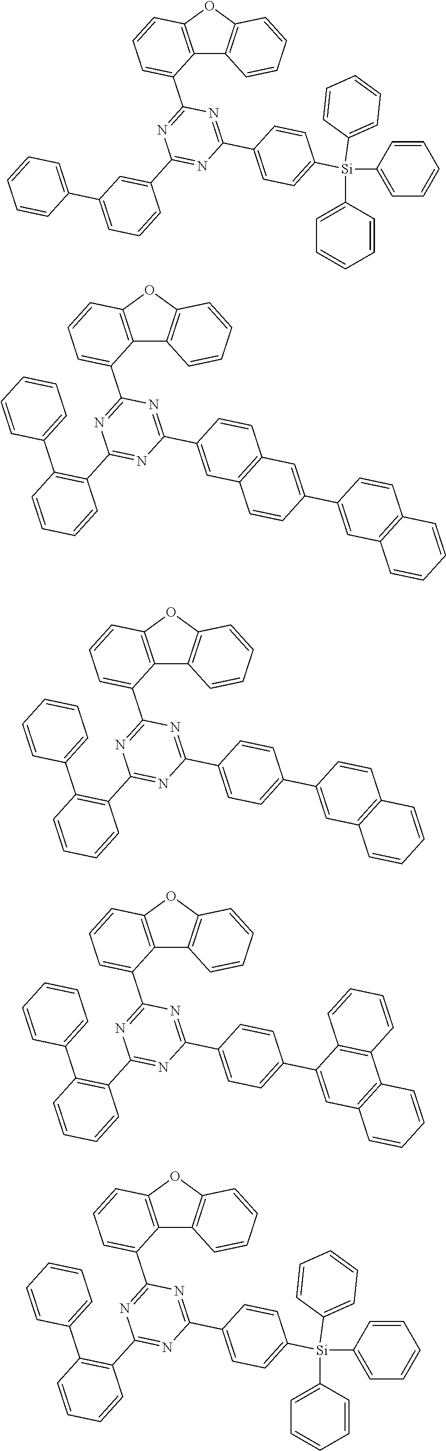 [SMILES: c1ccc(-c2cccc(-c3nc(-c4ccc([Si](c5ccccc5)(c5ccccc5)c5ccccc5)cc4)nc(-c4cccc5oc6ccccc6c45)n3)c2)cc1 ; c1ccc(-c2ccccc2-c2nc(-c3ccc4cc(-c5ccc6ccccc6c5)ccc4c3)nc(-c3cccc4oc5ccccc5c34)n2)cc1 ; c1ccc(-c2ccccc2-c2nc(-c3ccc(-c4ccc5ccccc5c4)cc3)nc(-c3cccc4oc5ccccc5c34)n2)cc1 ; c1ccc(-c2ccccc2-c2nc(-c3ccc(-c4cc5ccccc5c5ccccc45)cc3)nc(-c3cccc4oc5ccccc5c34)n2)cc1 ; c1ccc(-c2ccccc2-c2nc(-c3ccc([Si](c4ccccc4)(c4ccccc4)c4ccccc4)cc3)nc(-c3cccc4oc5ccccc5c34)n2)cc1]

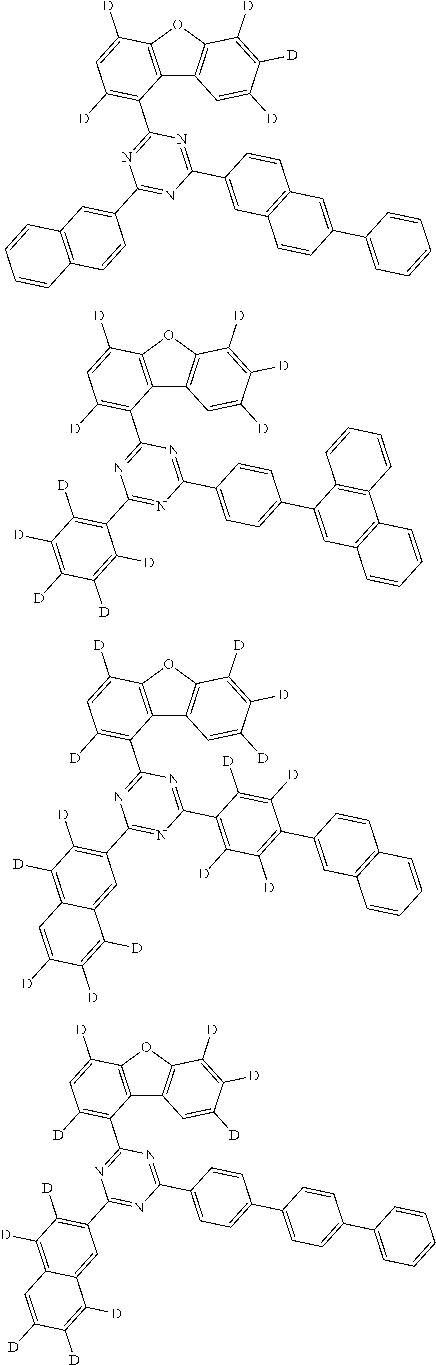

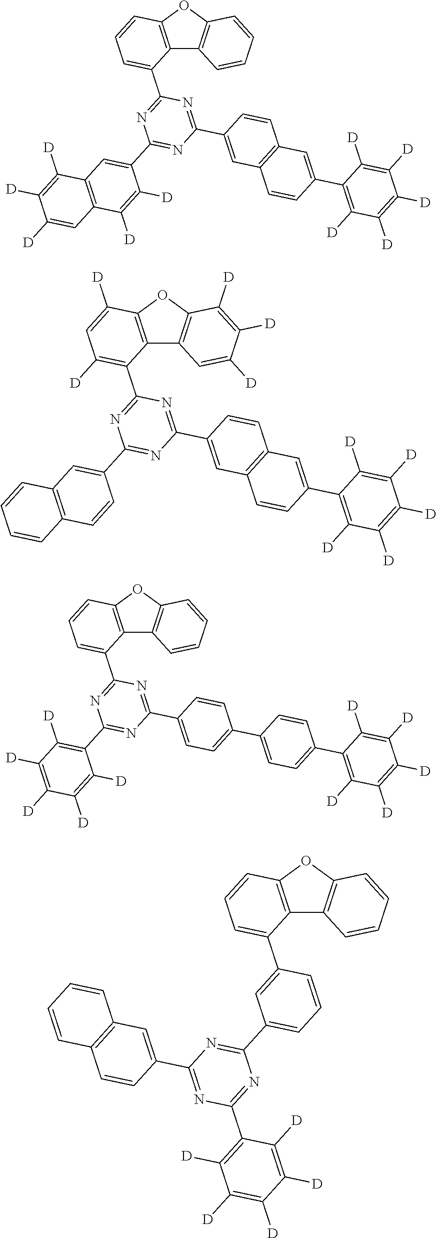 [SMILES: [2H]c1cc2c([2H])c([2H])c(-c3nc(-c4ccc5cc(-c6c([2H])c([2H])c([2H])c([2H])c6[2H])ccc5c4)nc(-c4cccc5oc6ccccc6c45)n3)cc2c([2H])c1[2H] ; [2H]c1cc2c(oc3c([2H])cc([2H])c(-c4nc(-c5ccc6ccccc6c5)nc(-c5ccc6cc(-c7c([2H])c([2H])c([2H])c([2H])c7[2H])ccc6c5)n4)c32)c([2H])c1[2H] ; [2H]c1c([2H])c([2H])c(-c2ccc(-c3ccc(-c4nc(-c5c([2H])c([2H])c([2H])c([2H])c5[2H])nc(-c5cccc6oc7ccccc7c56)n4)cc3)cc2)c([2H])c1[2H] ; [2H]c1c([2H])c([2H])c(-c2nc(-c3cccc(-c4cccc5oc6ccccc6c45)c3)nc(-c3ccc4ccccc4c3)n2)c([2H])c1[2H]]

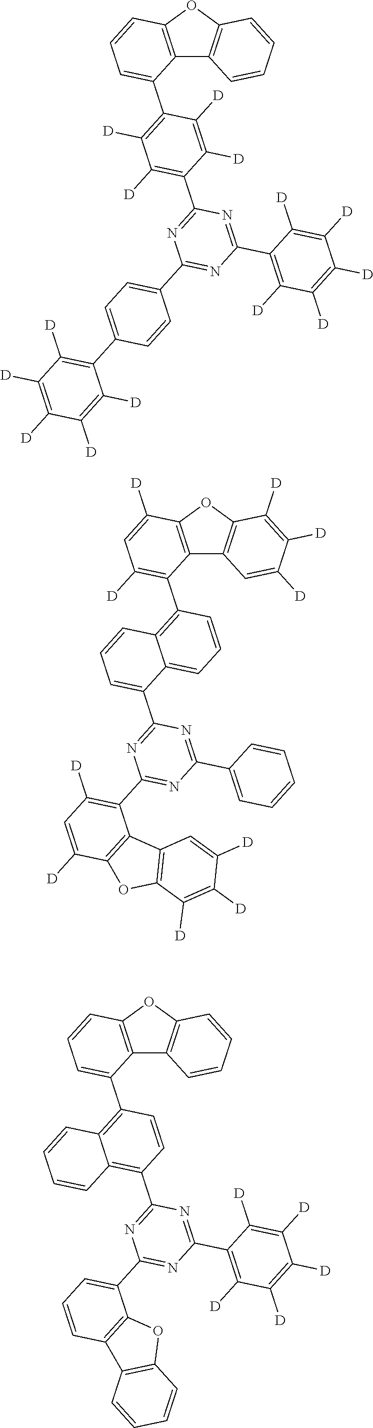 [SMILES: [2H]c1c([2H])c([2H])c(-c2ccc(-c3nc(-c4c([2H])c([2H])c([2H])c([2H])c4[2H])nc(-c4c([2H])c([2H])c(-c5cccc6oc7ccccc7c56)c([2H])c4[2H])n3)cc2)c([2H])c1[2H] ; [2H]c1cc2c(oc3c([2H])cc([2H])c(-c4nc(-c5ccccc5)nc(-c5cccc6c(-c7c([2H])cc([2H])c8oc9c([2H])c([2H])c([2H])cc9c78)cccc56)n4)c32)c([2H])c1[2H] ; [2H]c1c([2H])c([2H])c(-c2nc(-c3ccc(-c4cccc5oc6ccccc6c45)c4ccccc34)nc(-c3cccc4c3oc3ccccc34)n2)c([2H])c1[2H]]

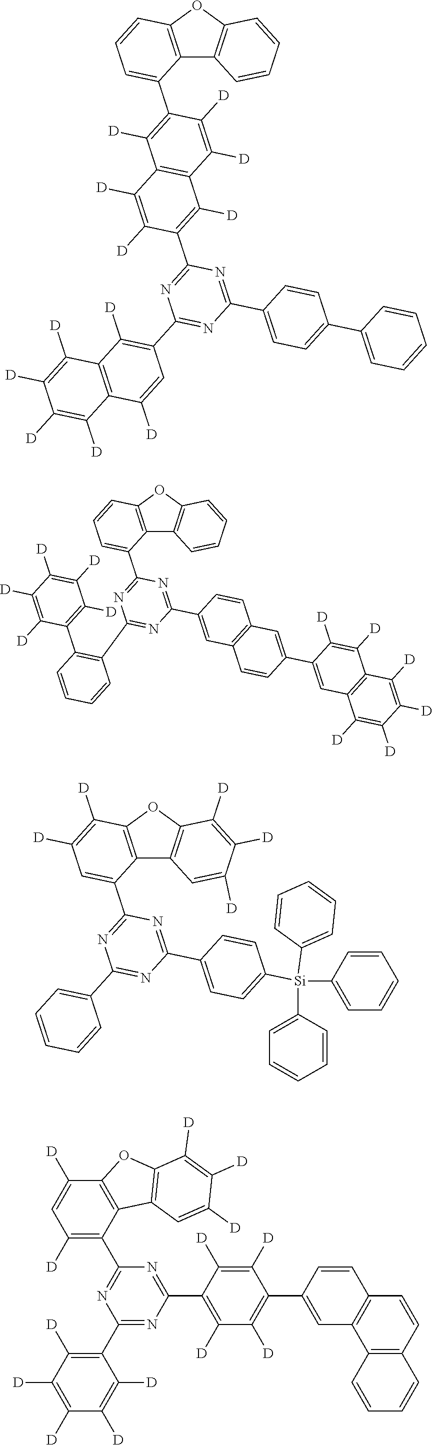

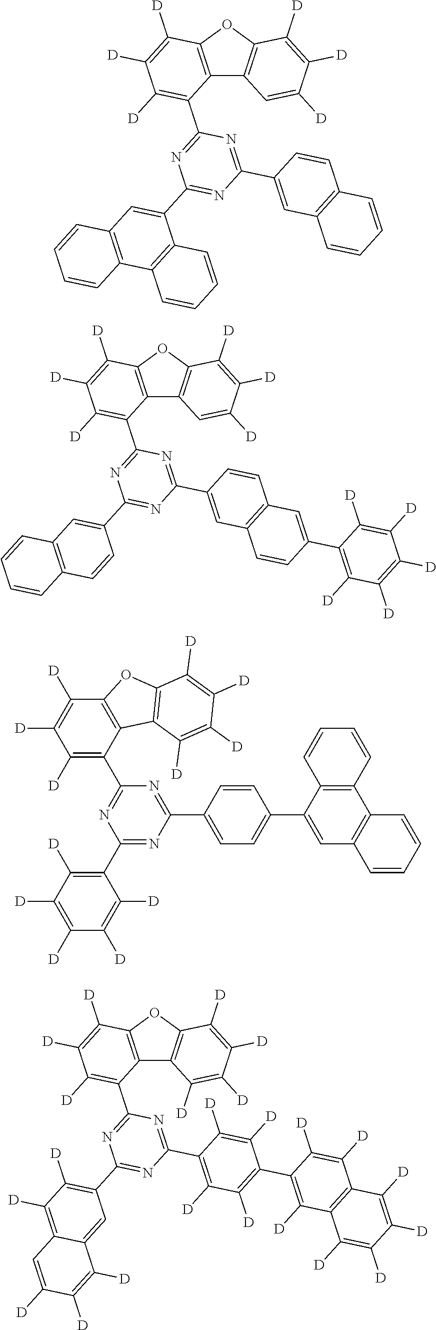

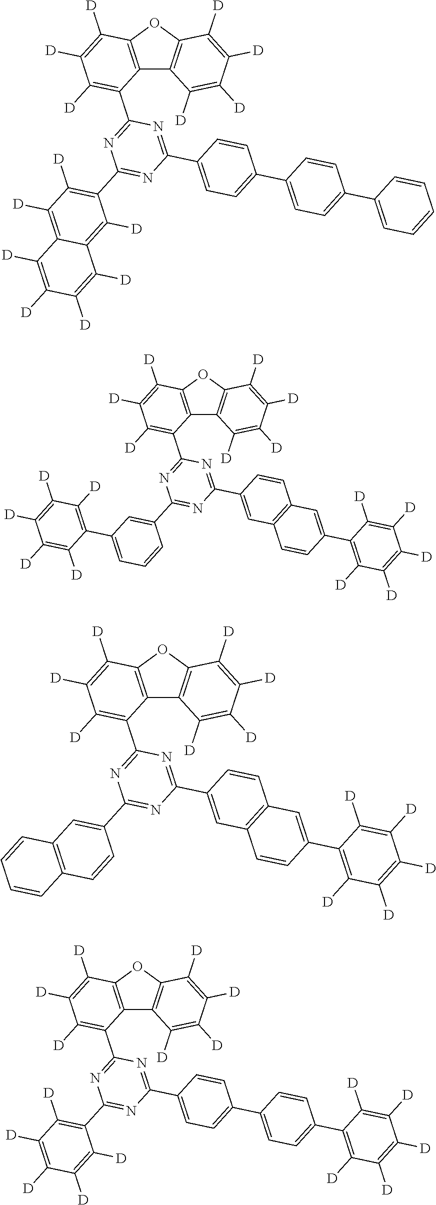

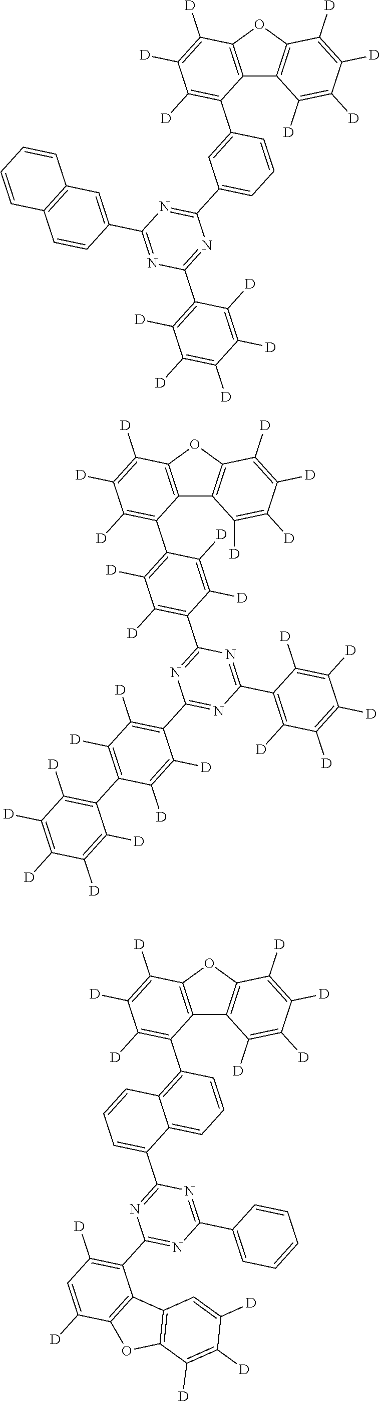

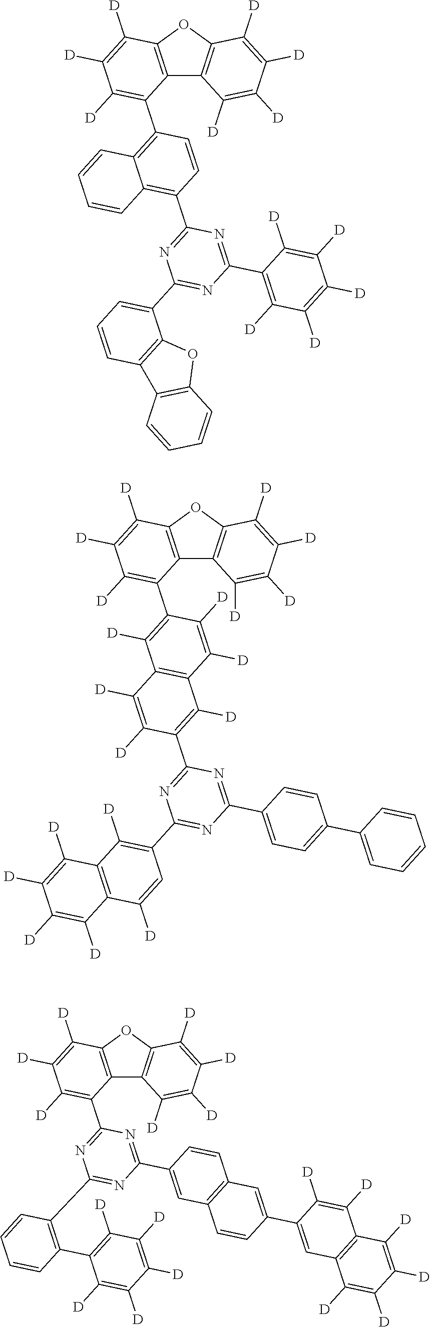

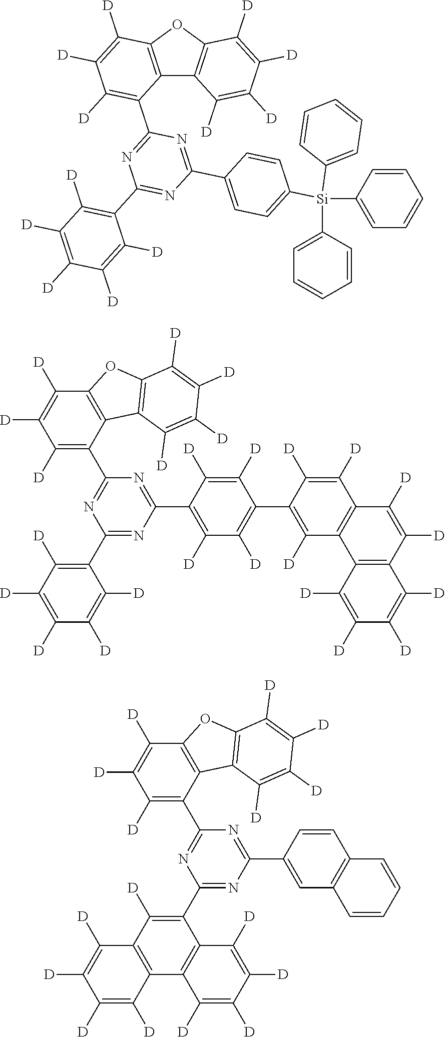

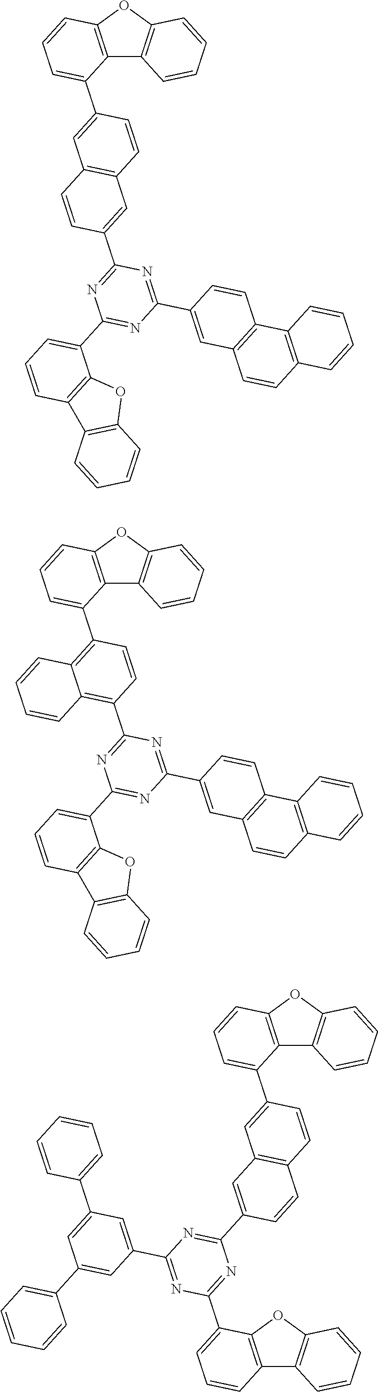 [SMILES: c1ccc2c(c1)ccc1cc(-c3nc(-c4ccc5cc(-c6cccc7oc8ccccc8c67)ccc5c4)nc(-c4cccc5c4oc4ccccc45)n3)ccc12 ; c1ccc2c(c1)ccc1cc(-c3nc(-c4ccc(-c5cccc6oc7ccccc7c56)c5ccccc45)nc(-c4cccc5c4oc4ccccc45)n3)ccc12 ; c1ccc(-c2cc(-c3ccccc3)cc(-c3nc(-c4ccc5ccc(-c6cccc7oc8ccccc8c67)cc5c4)nc(-c4cccc5c4oc4ccccc45)n3)c2)cc1]

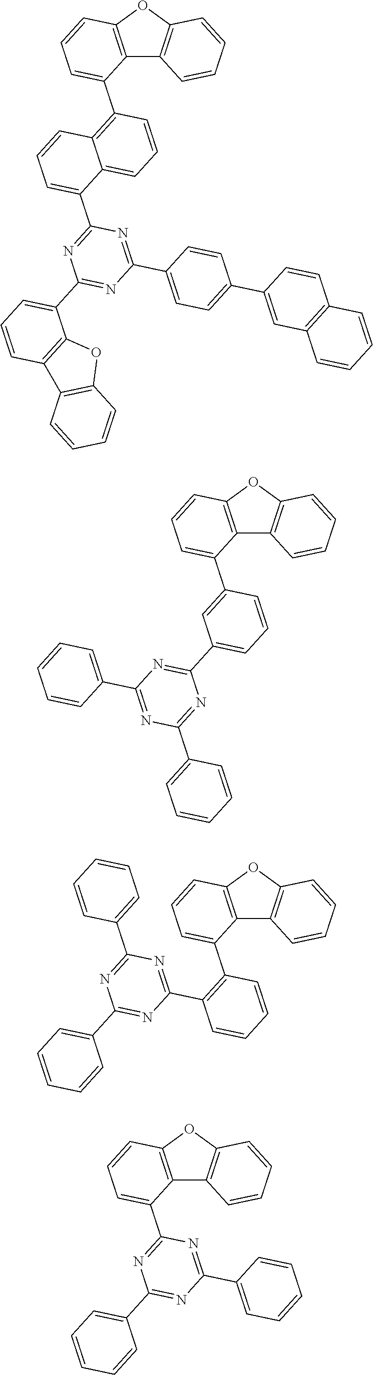 [SMILES: c1ccc2cc(-c3ccc(-c4nc(-c5cccc6c(-c7cccc8oc9ccccc9c78)cccc56)nc(-c5cccc6c5oc5ccccc56)n4)cc3)ccc2c1 ; c1ccc(-c2nc(-c3ccccc3)nc(-c3cccc(-c4cccc5oc6ccccc6c45)c3)n2)cc1 ; c1ccc(-c2nc(-c3ccccc3)nc(-c3ccccc3-c3cccc4oc5ccccc5c34)n2)cc1 ; c1ccc(-c2nc(-c3ccccc3)nc(-c3cccc4oc5ccccc5c34)n2)cc1]

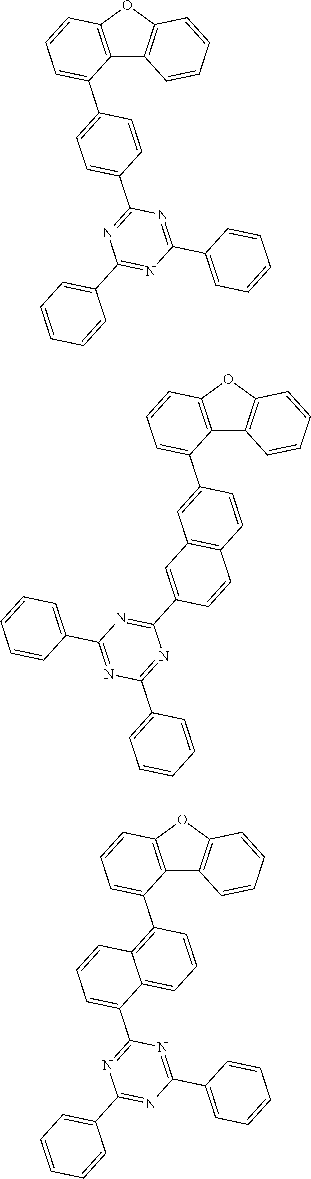

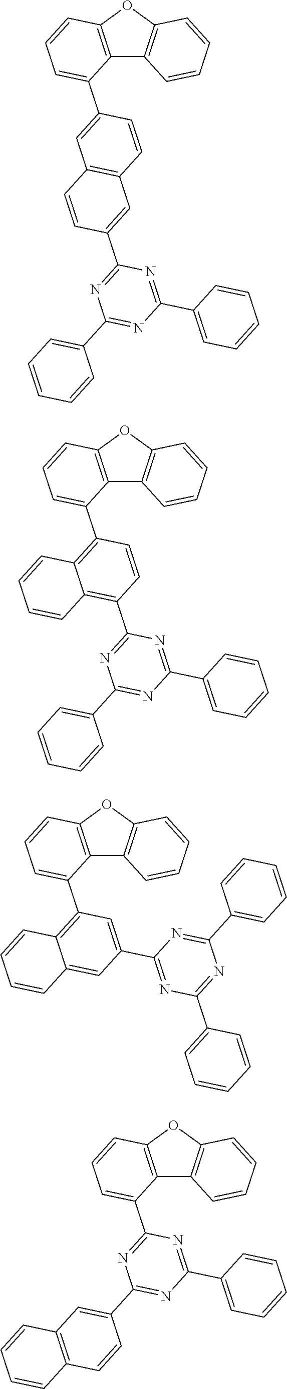

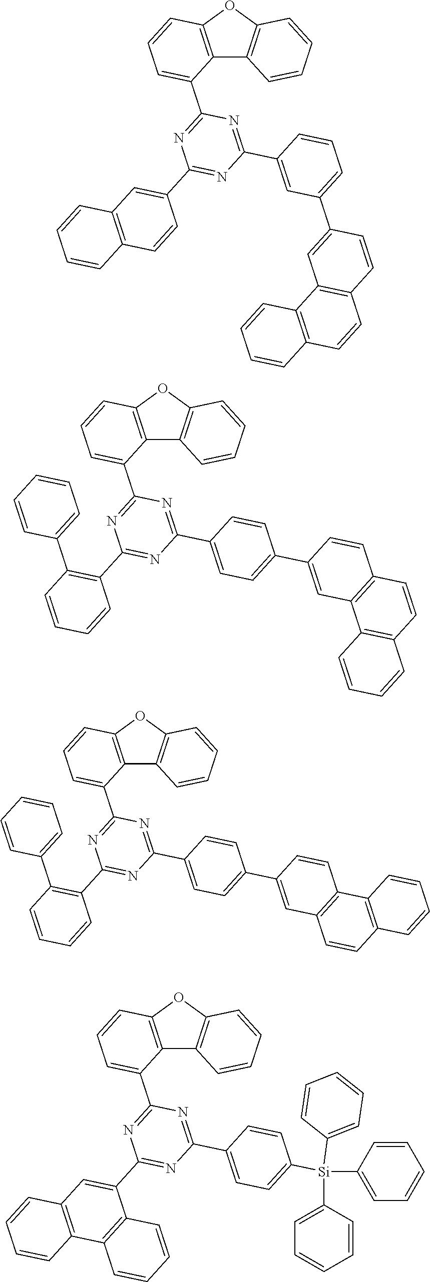 [SMILES: c1cc(-c2ccc3ccc4ccccc4c3c2)cc(-c2nc(-c3ccc4ccccc4c3)nc(-c3cccc4oc5ccccc5c34)n2)c1 ; c1ccc(-c2ccccc2-c2nc(-c3ccc(-c4ccc5ccc6ccccc6c5c4)cc3)nc(-c3cccc4oc5ccccc5c34)n2)cc1 ; c1ccc(-c2ccccc2-c2nc(-c3ccc(-c4ccc5c(ccc6ccccc65)c4)cc3)nc(-c3cccc4oc5ccccc5c34)n2)cc1 ; c1ccc([Si](c2ccccc2)(c2ccccc2)c2ccc(-c3nc(-c4cc5ccccc5c5ccccc45)nc(-c4cccc5oc6ccccc6c45)n3)cc2)cc1]

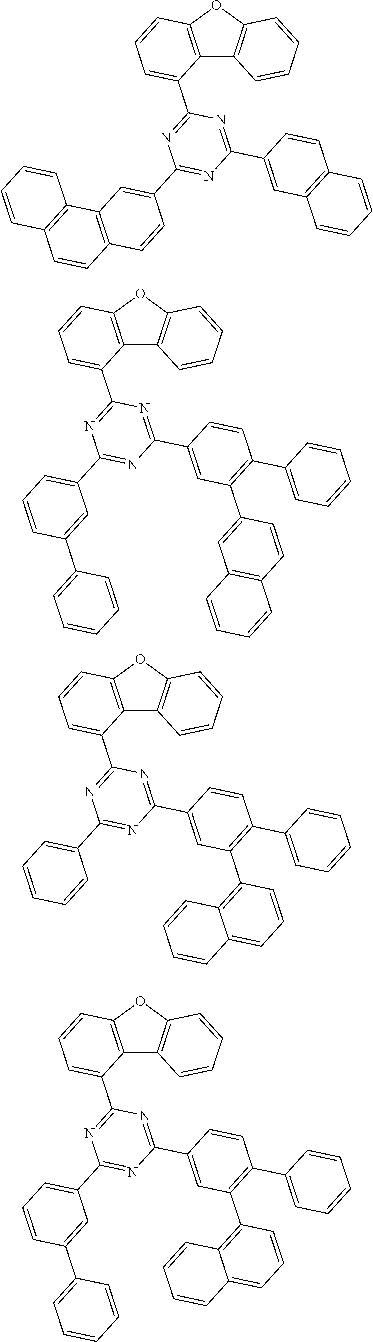

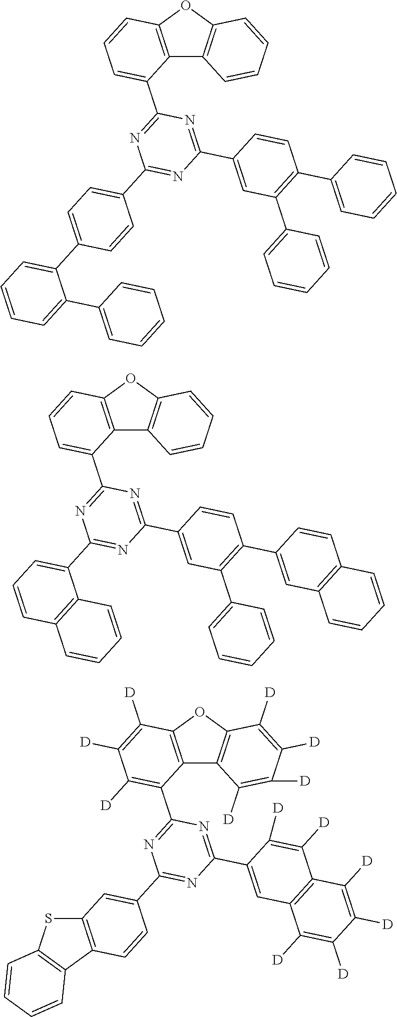

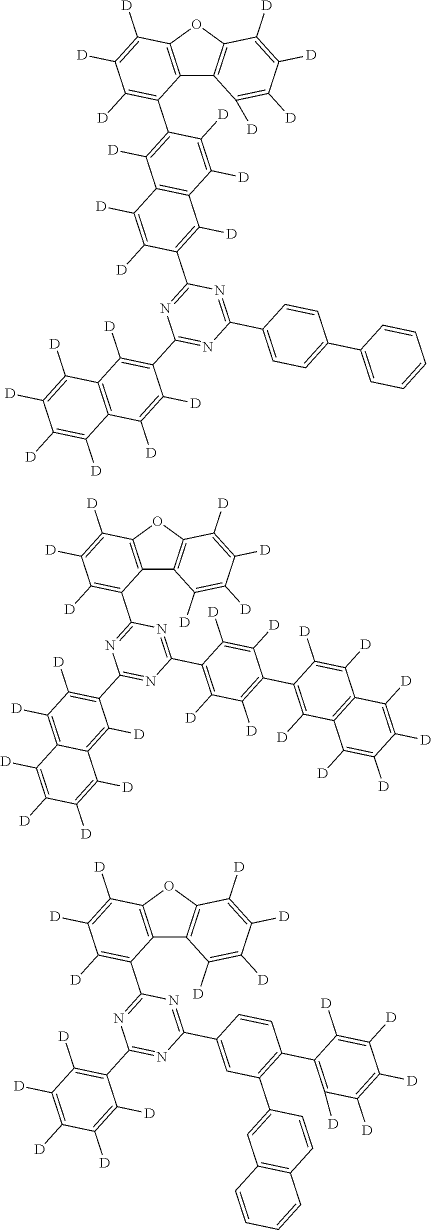

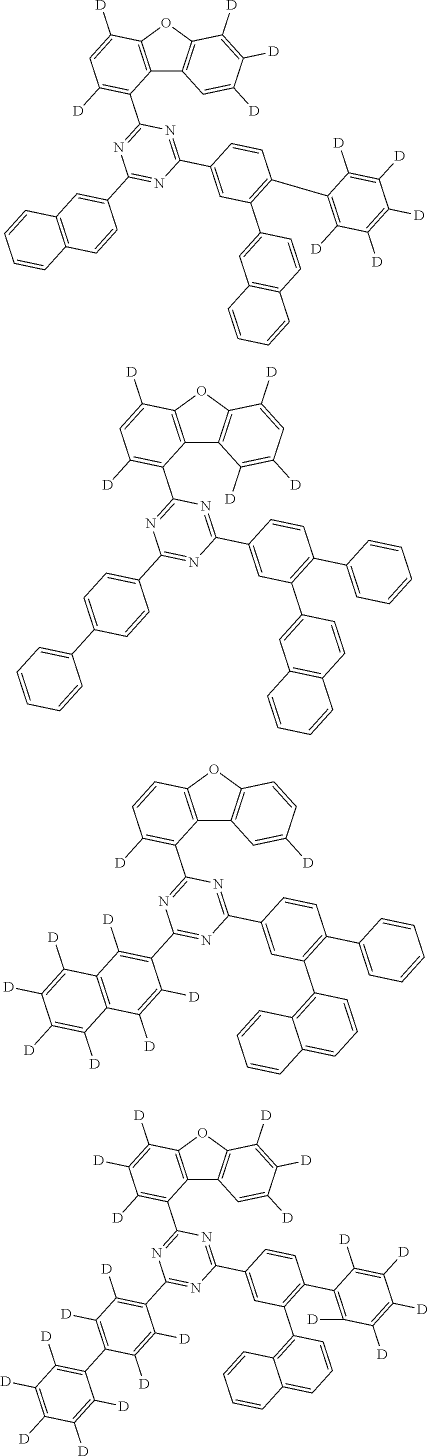

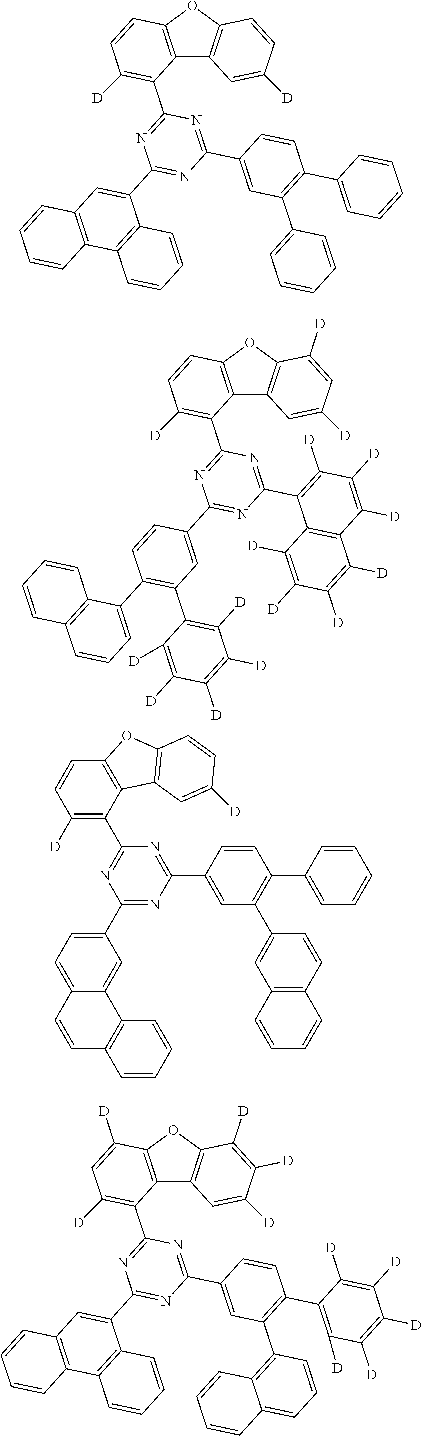

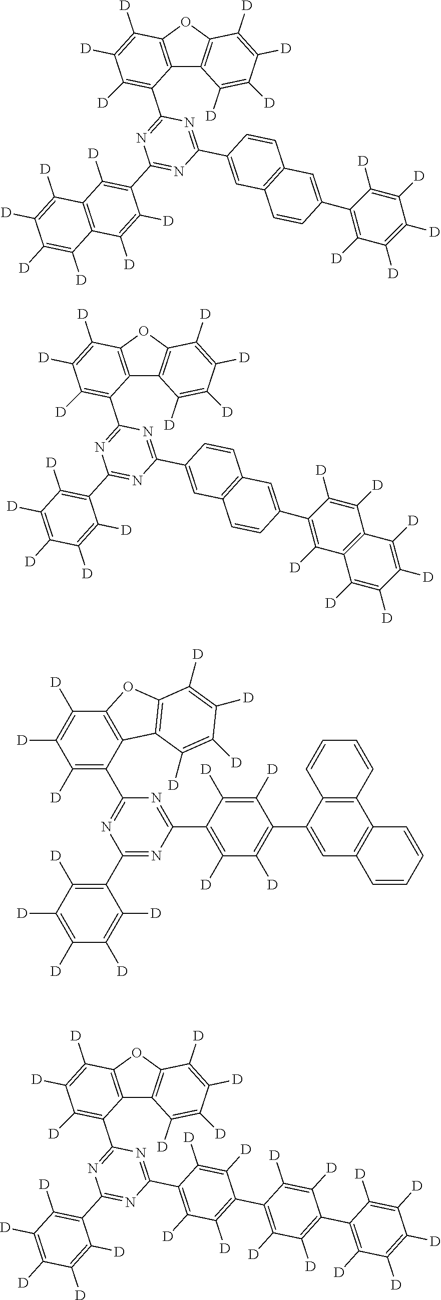

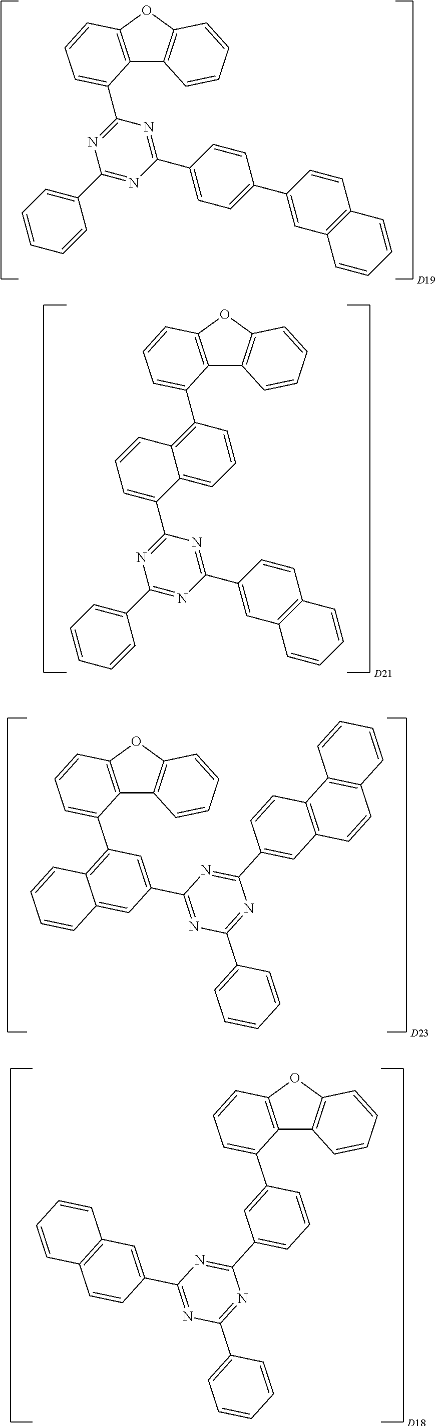

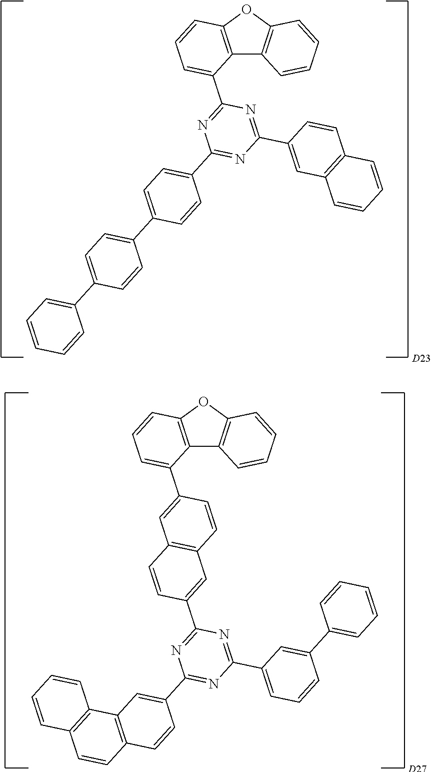

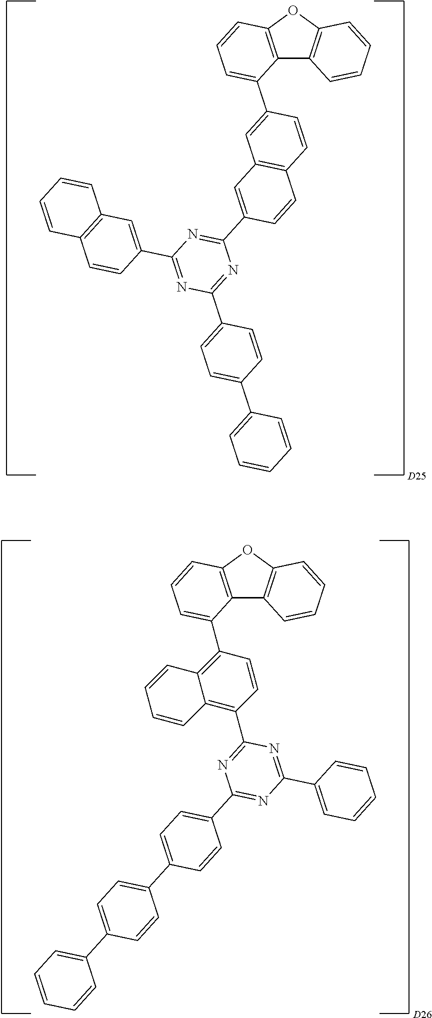

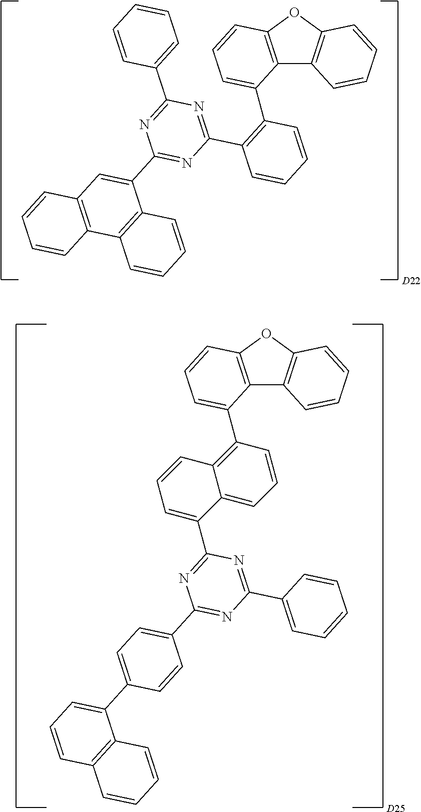

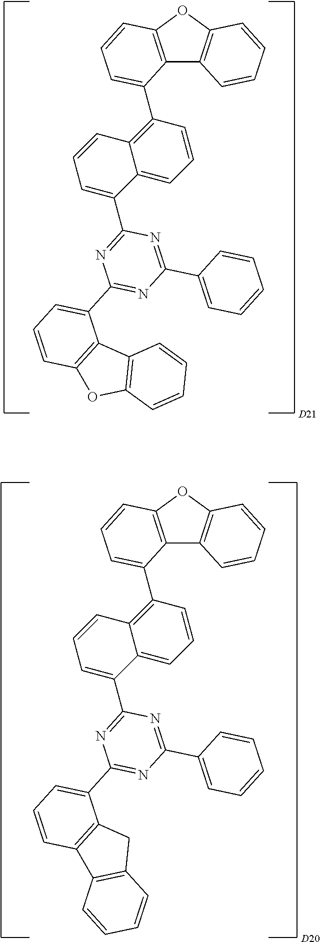

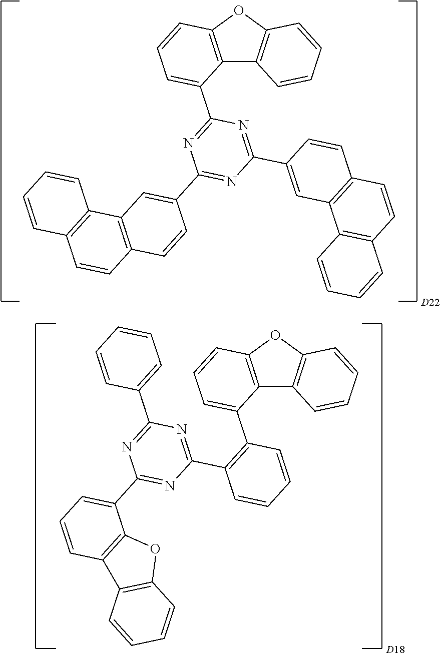

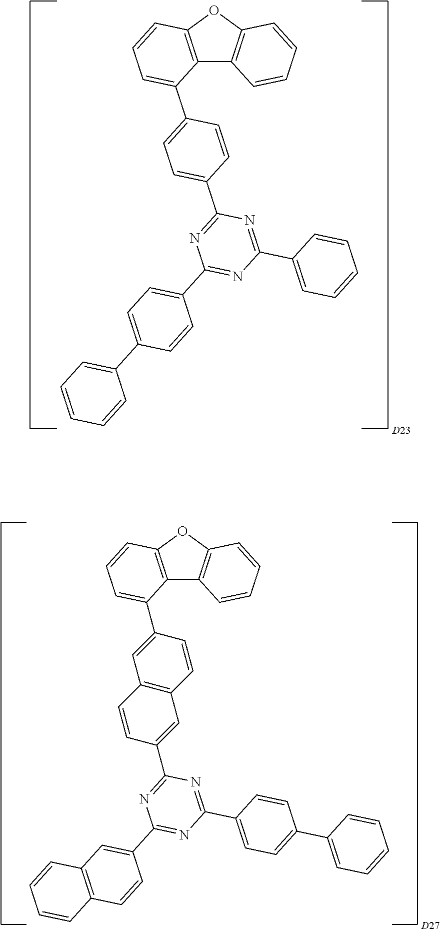

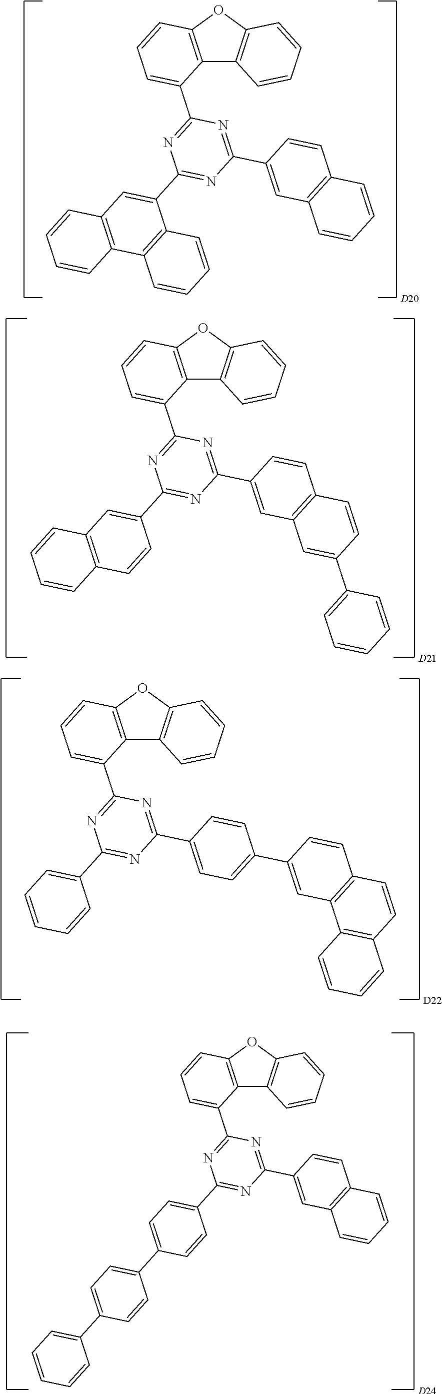

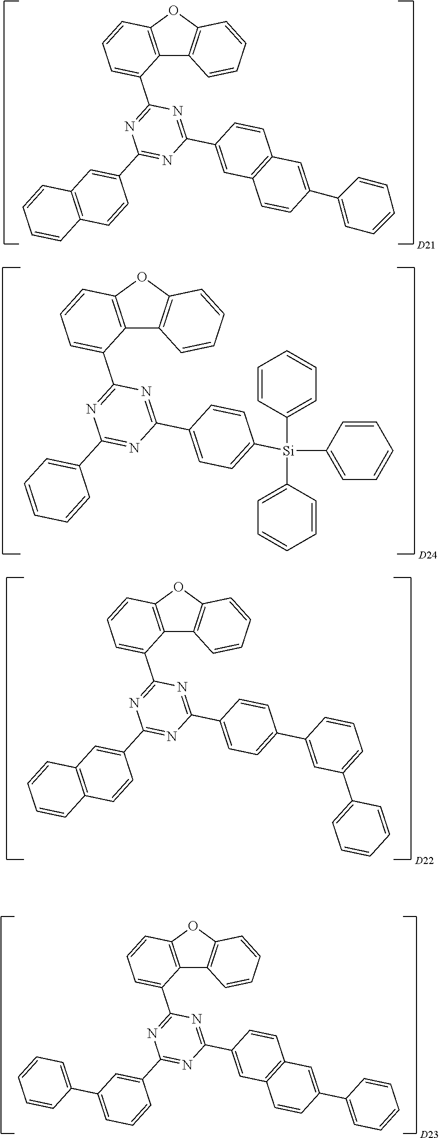

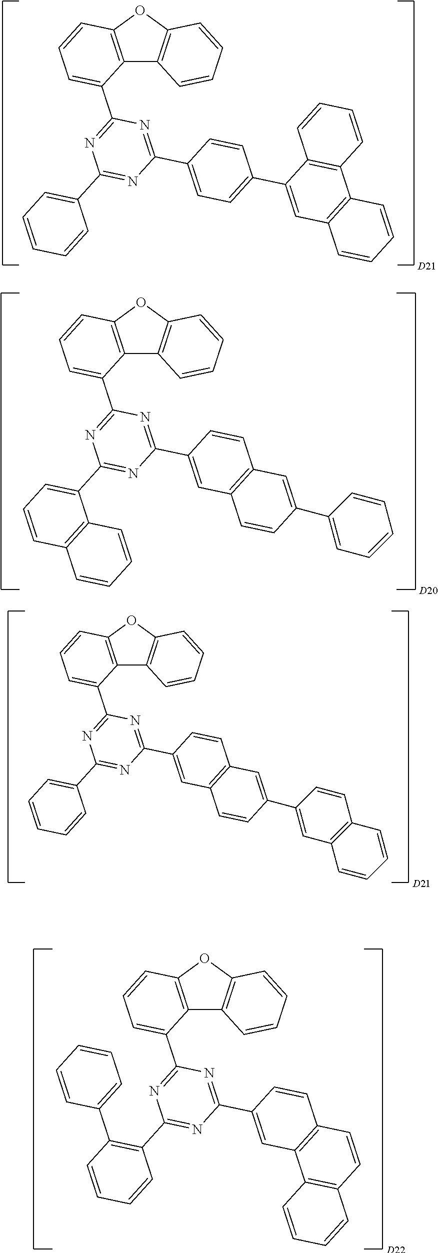

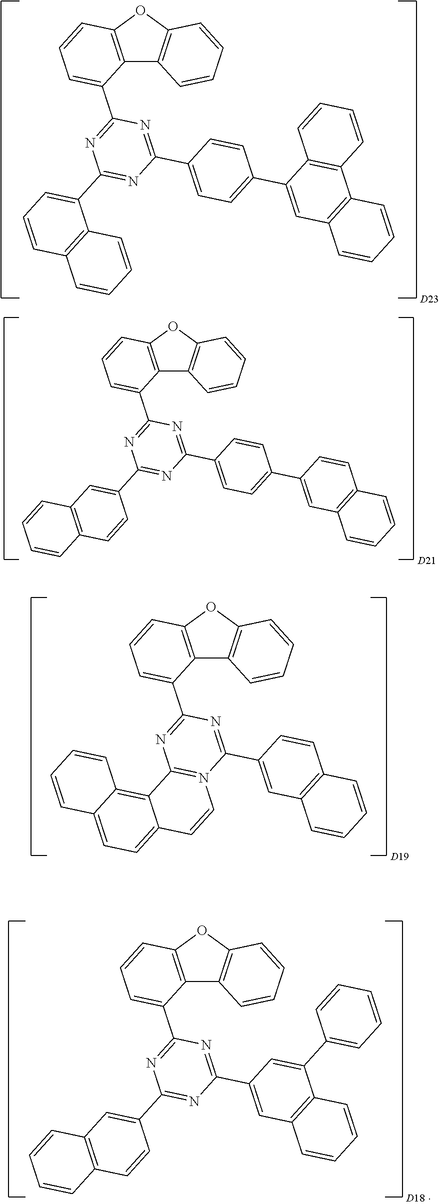

Meanwhile, the first compound can be prepared by a preparation method as shown in the following Reaction Scheme 1:

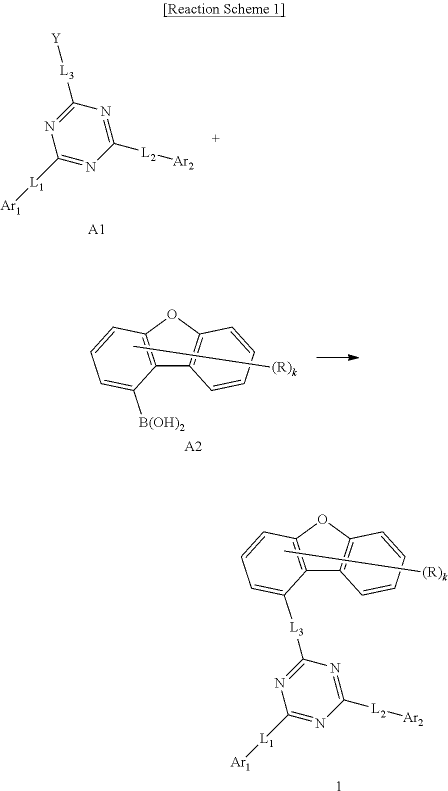

In Reaction Scheme 1, Y is halogen, preferably bromo, or chloro, and the definitions of other substituents are the same as described above.

Specifically, the first compound may be prepared by a Suzuki coupling reaction of the starting materials A1 and A2. Such a Suzuki coupling reaction is preferably carried out in the presence of a palladium catalyst and a base, and a reactive group for the Suzuki coupling reaction can be appropriately changed. The preparation method of the first compound can be further embodied in Preparation Examples described hereinafter.

### (Second Compound)

The second compound is represented by Chemical Formula 2 above. Specifically, the second compound is a tertiary amine compound having two benzonaphthofuranyl/benzonaphthothiophenyl substituents, wherein the compound has a structure in which one of the benzonaphthofuranyl/benzonaphthothiophenyl substituents is a benzene ring which is linked to the nitrogen atom of the amino group, and the other one of the benzonaphthofuranyl/benzonaphthothiophenyl substituents is a naphthalene ring which is linked to the nitrogen atom of the amino group. The compound represented by Chemical Formula 1 having such a structure can efficiently transfer holes to a dopant material, and thus increase the recombination probabilities of holes and electrons in the light emitting layer together with the first compound having excellent electron transport capability.

Meanwhile, the second compound may be represented by any one of the following Chemical Formulas 1A to 1C depending on the fusion position of the A₁ ring:

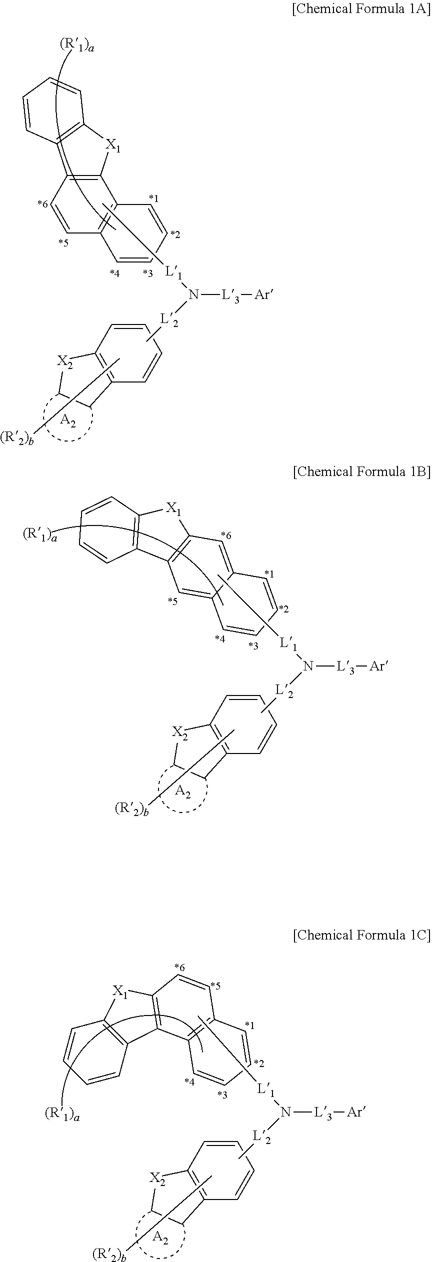

wherein in Chemical Formulas 1A to 1C,
L₁ is linked to any one of carbon at position *1, carbon at position *2, carbon at position *3, carbon at position *4, carbon at position *5, and carbon at position *6, and
X₁, X₂, A₂, L′₁ to L′₃, Ar′, R′₁, R′₂, a and b are as defined in Chemical Formula 1.

In one embodiment, both X₁ and X₂ are O; or
both X₁ and X₂ are S; or
X₁ is O and X₂ is S; or
X₁ may be S, and X₂ may be O.

In one embodiment, the second compound may be represented by the following Chemical Formula 2-1:

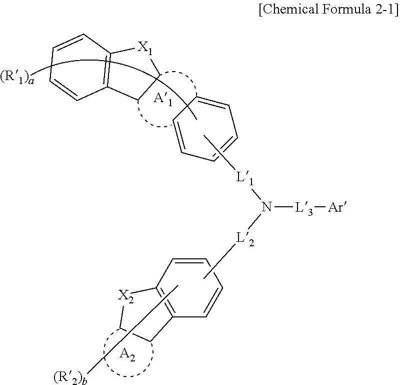

wherein in Chemical Formula 2-1,
A′₁ is a benzene ring fused with adjacent pentagonal and benzene rings, and
X₁, X₂, A₂, L′₁ to L′₃, Ar′, R′₁, R′₂, a and b are as defined in in Chemical Formula 1.

In one embodiment,

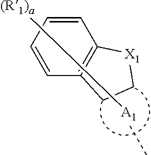

may be any one of the substituents represented by the following Chemical Formulas 2a to 2l:

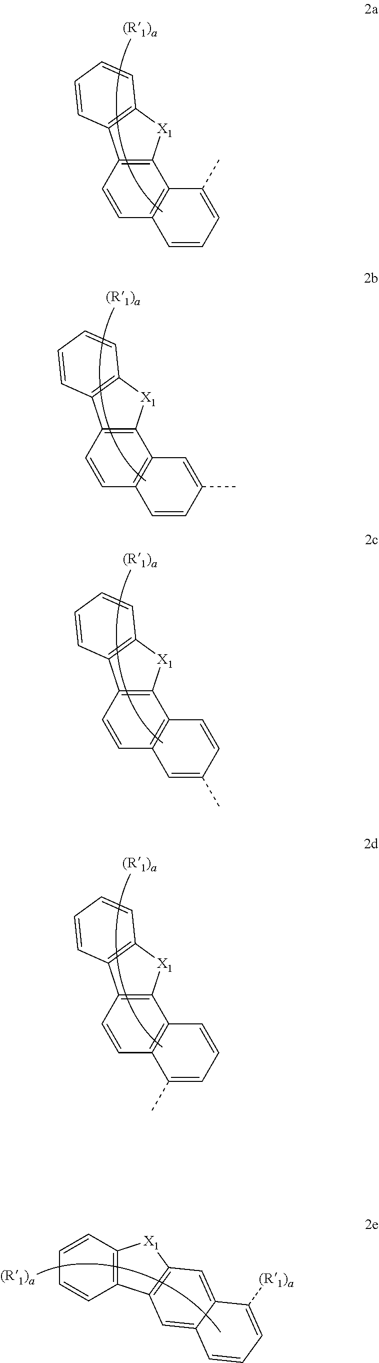

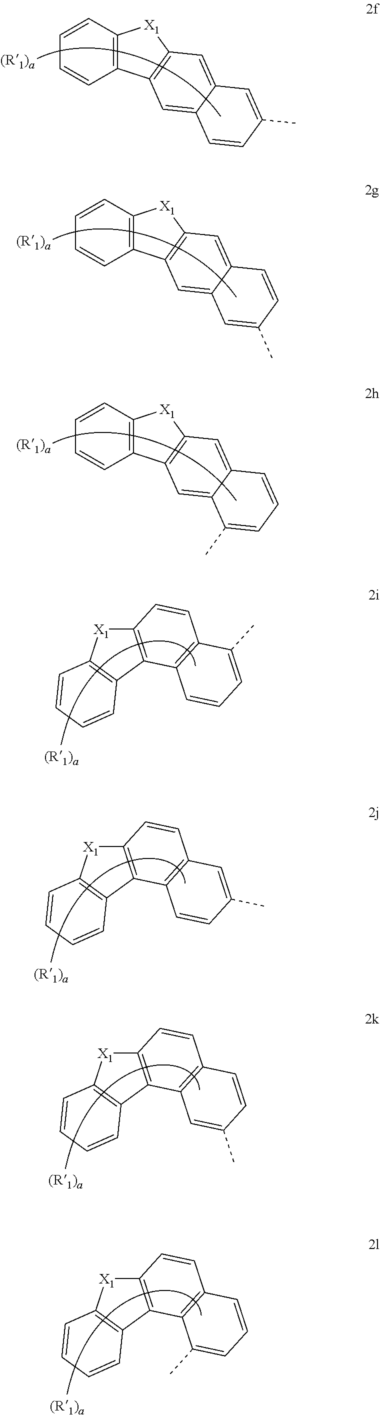

wherein in Chemical Formulas 2a to 2l,
X₁, R′₁ and a are as defined in Chemical Formula 1.

Further, in one embodiment,

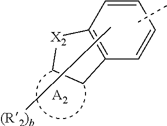

may be any one of the substituents represented by the following Chemical Formulas 3a to 3l:

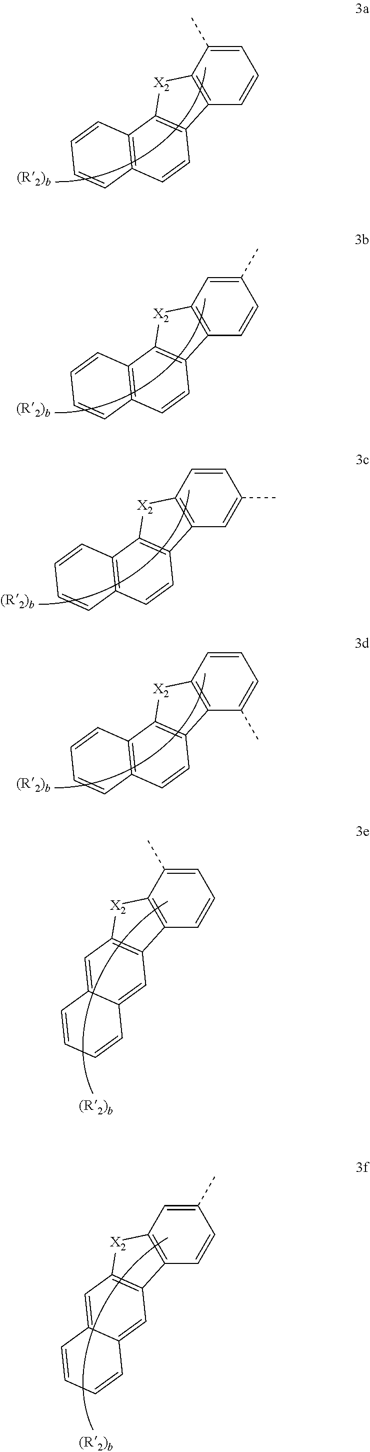

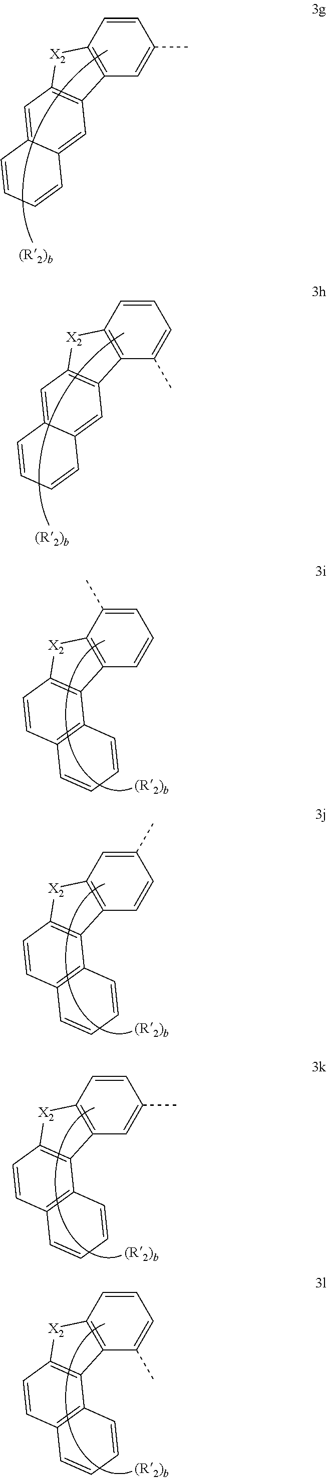

wherein in Chemical Formulas 3a to 3l,
X₂, R′₂ and b are as defined in Chemical Formula 1.

In one embodiment, L′₁ and L′₂ may be each independently a single bond, or a substituted or unsubstituted C₆₋₂₀ arylene.

In other embodiments, L′₁ and L′₂ may be each independently a single bond; or a C₆₋₂₀ arylene which is unsubstituted, or substituted with one or more substituents selected from the group consisting of deuterium, phenyl and naphthyl.

In yet another embodiment, L′₁ and L′₂ may be each independently a single bond, or a substituted or unsubstituted C₆₋₁₂ arylene.

In yet another embodiment, L′₁ and L′₂ may be each independently a single bond, a substituted or unsubstituted phenylene, a substituted or unsubstituted biphenyldiyl, or a substituted or unsubstituted naphthylene.

In yet another embodiment, L′₁ and L′₂ may be each independently a single bond, phenylene, biphenyldiyl, or naphthylene,
wherein the phenylene, biphenyldiyl, and naphthylene may be unsubstituted, or substituted with deuterium.

For example, L′₁ and L′₂ may be each independently a single bond, or any one selected from the group consisting of the following formulas and the deuterated structure thereof:

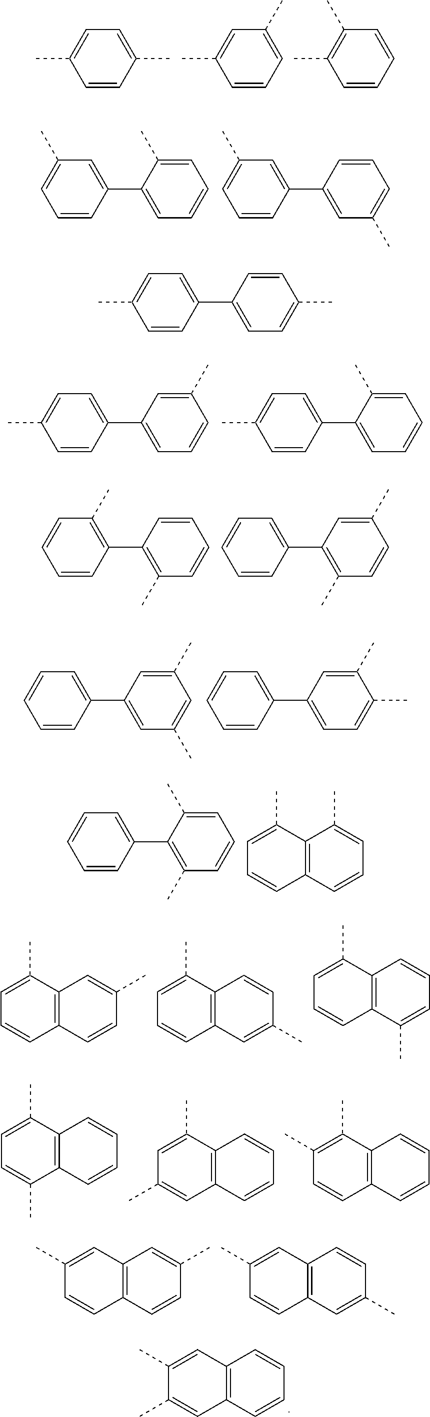

More specifically, for example, L′₁ and L′₂ may be each independently a single bond, or a substituted or unsubstituted 1,4-phenylene.

In yet another embodiment, at least one of L′₁ and L′₂ may be a single bond.

At this time, L′₁ and L′₂ may be identical to each other. Alternatively, L′₁ and L′₂ may be different from each other.

For example, both L′₁ and L′₂ are a single bond; or
L′₁ is a single bond, and L′₂ is a substituted or unsubstituted C₆₋₁₂ arylene; or
L′₁ is a substituted or unsubstituted C₆₋₁₂ arylene, and L′₂ may be a single bond.

For example, both L′₁ and L′₂ are a single bond; or
L′₁ is a single bond, and L′₂ is a substituted or unsubstituted C₆₋₁₀ arylene; or
L′₁ may be a substituted or unsubstituted C₆₋₁₀ arylene, and L′₂ may be a single bond.

Further, in one embodiment, L′₃ may be a single bond, or a substituted or unsubstituted C₆₋₂₀ arylene.

In other embodiments, L′₃ is a single bond; or a C₆₋₂₀ arylene which is unsubstituted, or substituted with one or more substituents selected from the group consisting of deuterium, phenyl, and naphthyl.

In yet another embodiment, L′₃ may be a single bond, a substituted or unsubstituted phenylene, a substituted or unsubstituted biphenyldiyl, or a substituted or unsubstituted naphthylene.

For example, L′₃ may be a single bond, or a substituted or unsubstituted phenylene.

Further, in one embodiment, Ar′ may be a substituted or unsubstituted C₆₋₂₀ aryl; or a substituted or unsubstituted C₂₋₂₀ heteroaryl containing a heteroatom of O or S.

In other embodiments, Ar′ is phenyl, biphenylyl, terphenylyl, naphthyl, phenanthryl, triphenylenyl, dibenzofuranyl, dibenzothiophenyl, benzonaphthofuranyl, or benzonaphthothiophenyl,
wherein the Ar′ may be substituted or unsubstituted.

In yet another embodiment, Ar′ is phenyl, biphenylyl, terphenylyl, naphthyl, phenanthryl, triphenylenyl, dibenzofuranyl, dibenzothiophenyl, benzonaphthofuranyl, or benzonaphthothiophenyl,
wherein the Ar′ may be unsubstituted, or substituted with one or more substituents selected from the group consisting of deuterium, halogen, cyano, phenyl substituted or unsubstituted with deuterium, biphenylyl substituted or unsubstituted with deuterium, and naphthyl substituted or unsubstituted with deuterium.

For example, Ar may be any one selected from the group consisting of the following formulas and the deuterated structure thereof, but is not limited thereto:

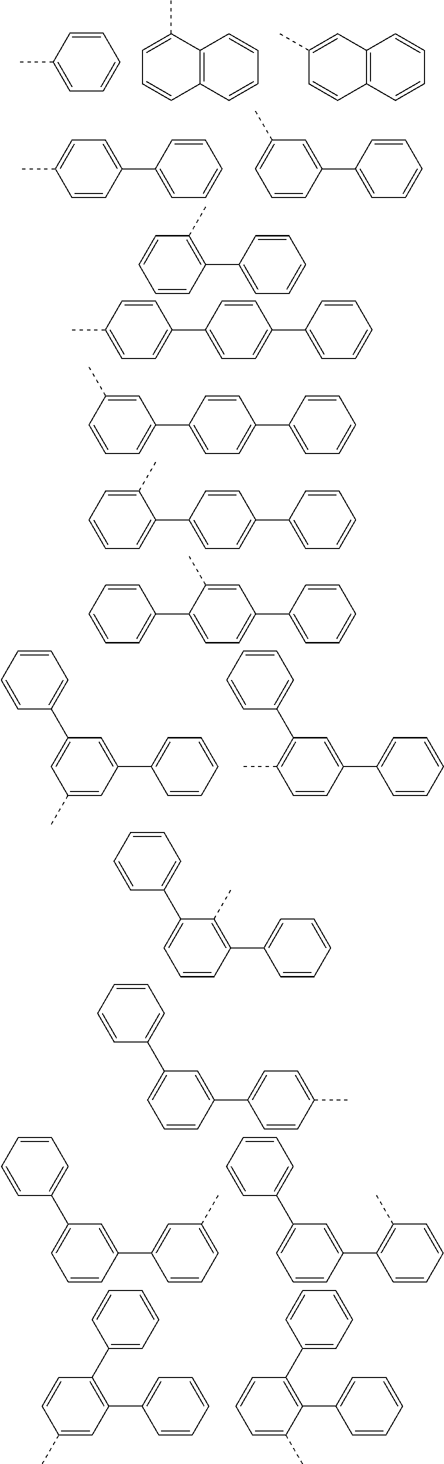

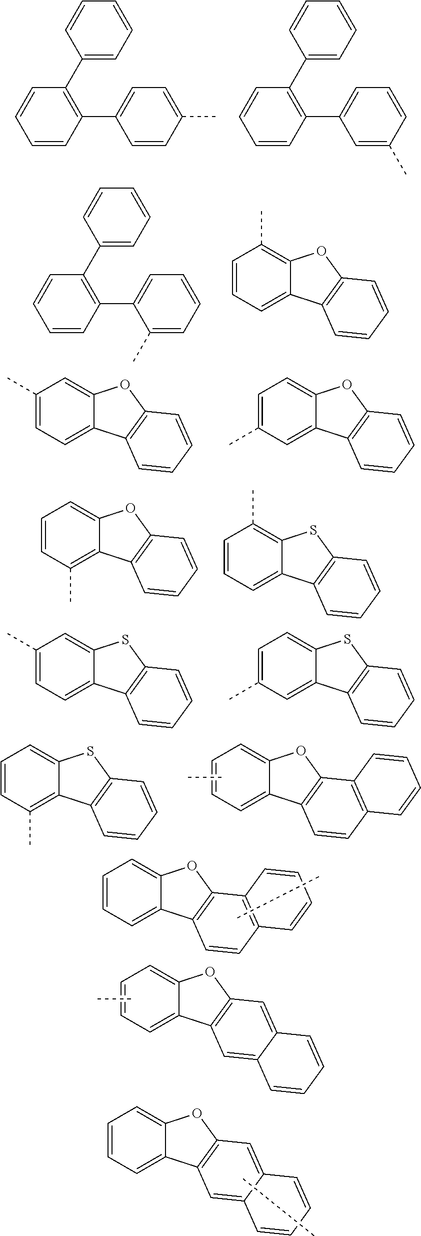

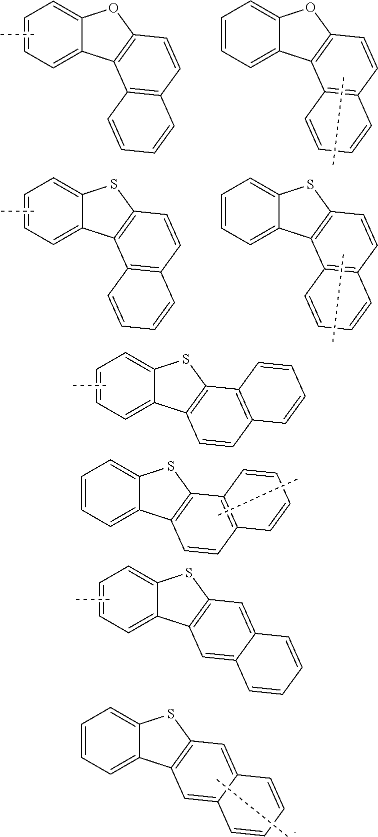

wherein a means the number of R′₁, and when a is 2 or more, two or more R′₁ may be the same as or different from each other. Specifically, a is 0, 1, 2, 3, 4, 5, 6, 7, 8, or 9.

Further, b means the number of R′₂, and when b is 2 or more, two or more R′₂ may be the same as or different from each other. Specifically, b is 0, 1, 2, 3, 4, 5, 6, 7, 8, or 9.

For example, both R′₁ and R′₂ may be hydrogen; or both may be deuterium.

Further, the second compound may be represented by any one of the following Chemical Formulas 2-1 to 2-9:

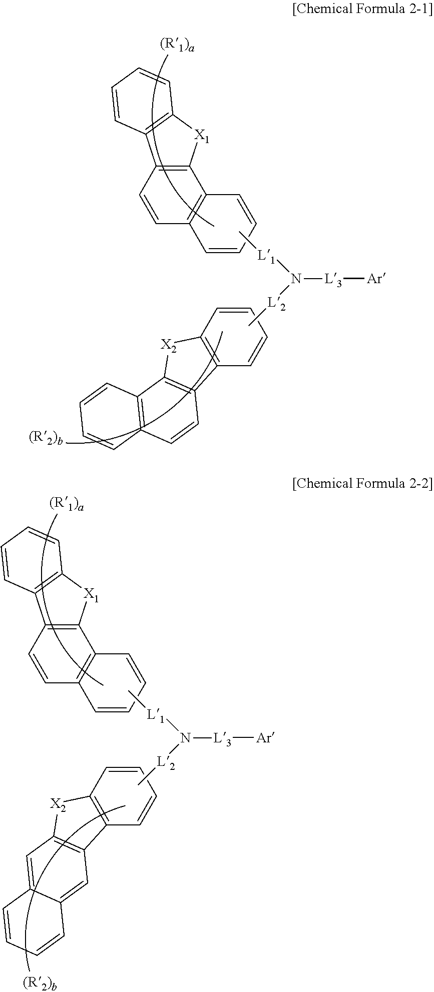

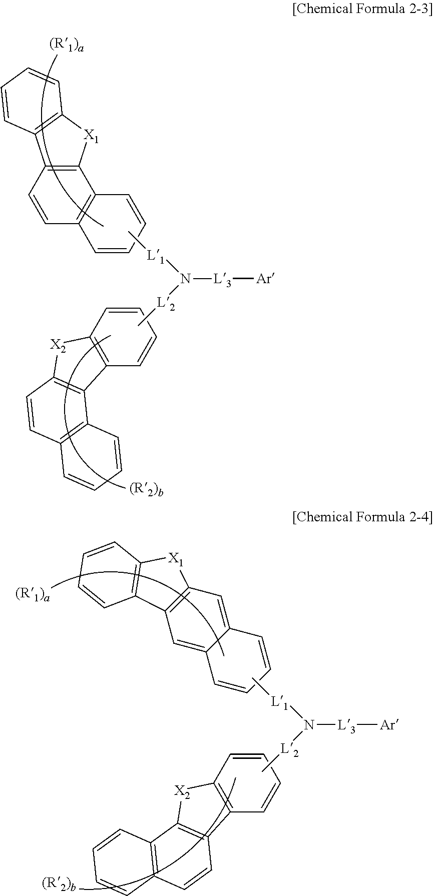

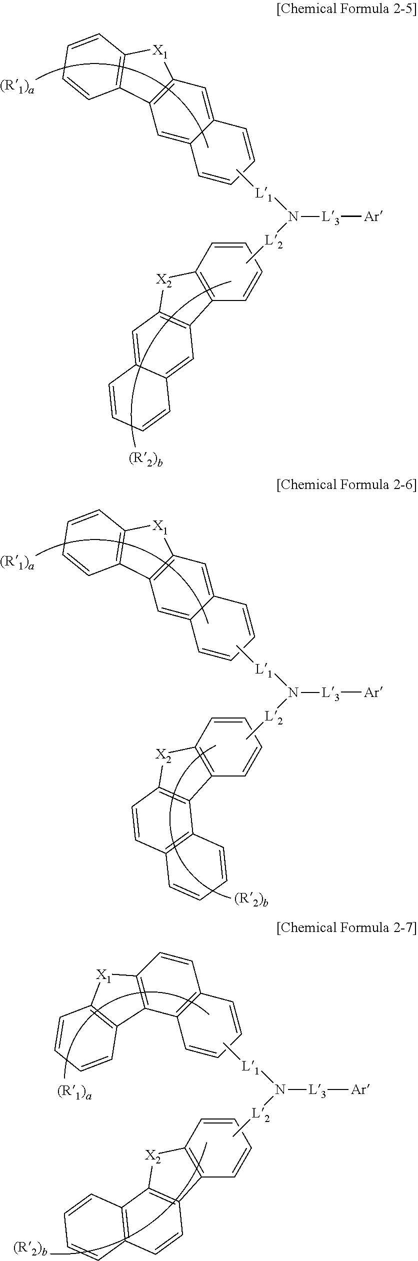

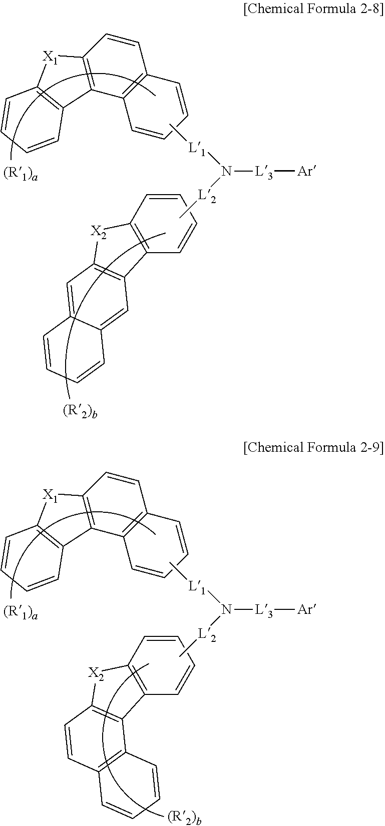

wherein in Chemical Formulas 2-1 to 2-9,
X₁, X₂, L′₁ to L′₃, Ar′, R′₁, R′₂, a and b are as defined in Chemical Formula 1.

Further, the second compound may not contain deuterium, or may contain at least one deuterium.

When the second compound contains deuterium, the deuterium substitution rate of the second compound may be 1% to 100%. Specifically, the deuterium substitution rate of the second compound may be 5% or more, 10% or more, 20% or more, 30% or more, 40% or more, 50% or more, 60% or more, 70% or more, 75% or more, 80% or more, or 90% or more, and 100% or less.

In one embodiment, the second compound may not contain deuterium, or may contain 1 to 50 deuterium atoms. More specifically, the second compound may not contain deuterium, or may contain 1 or more, 2 or more, 3 or more, 4 or more, 5 or more, 6 or more, 7 or more, 8 or more, or 9 or more, and 50 or less, 40 or less, 30 or less, 28 or less, 26 or less, 24 or less, 22 or less, 20 or less, 18 or less, 16 or less, 14 or less, 13 or less, 12 or less, 11 or less, or 10 or less deuterium atoms.

In this case, when the number of deuterium substitution of the second compound is to be represented, it may be represented by the following Chemical Formula 2D:

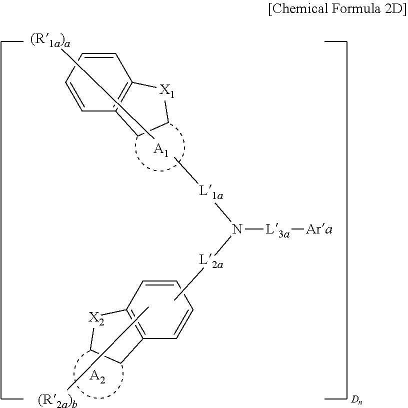

wherein in Chemical Formula 2D,
Dn means that n hydrogens have been replaced by deuterium,
wherein n is an integer of 0 or more,
L′₁ₐ to L′₃ₐ, Ar′ₐ, R′₁ₐ and R′₂ₐ mean L′₁ to L′₃, Ar′, R′₁ and R′₂ substituents which are not substituted with deuterium, respectively.

Meanwhile, representative examples of the second compound are as follows:

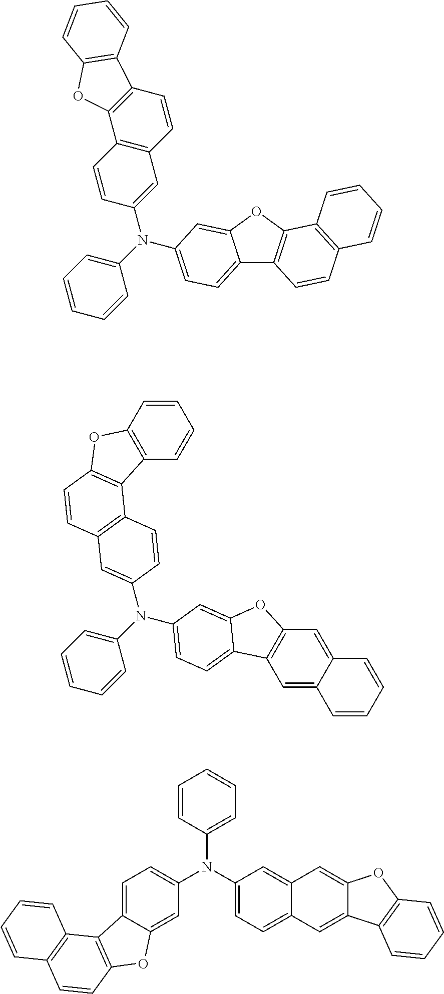

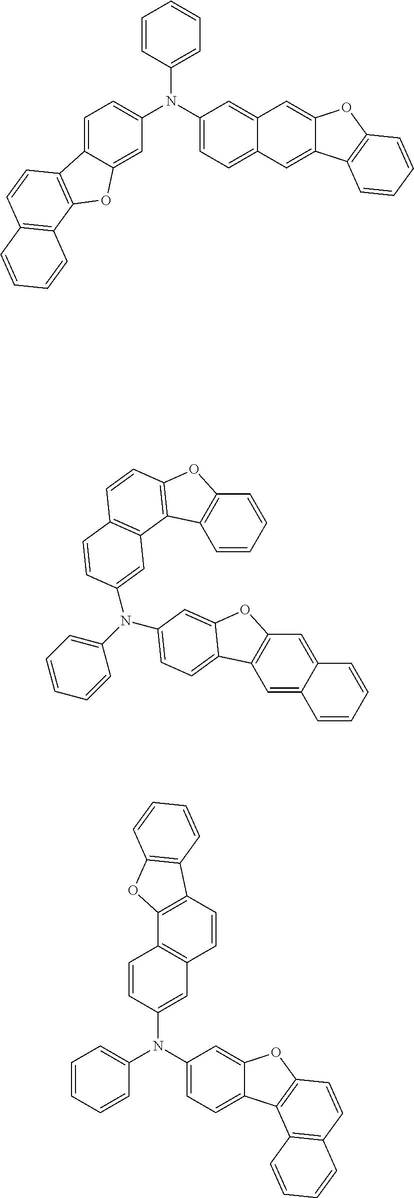

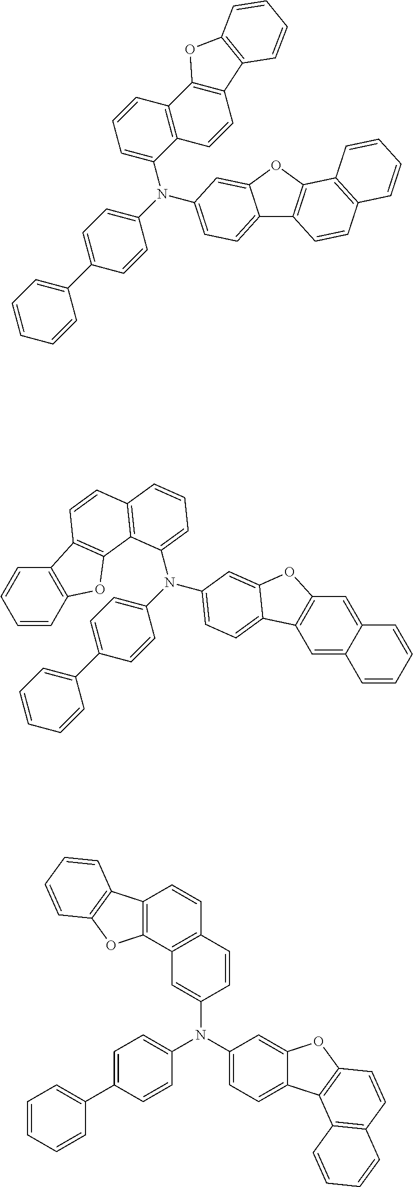

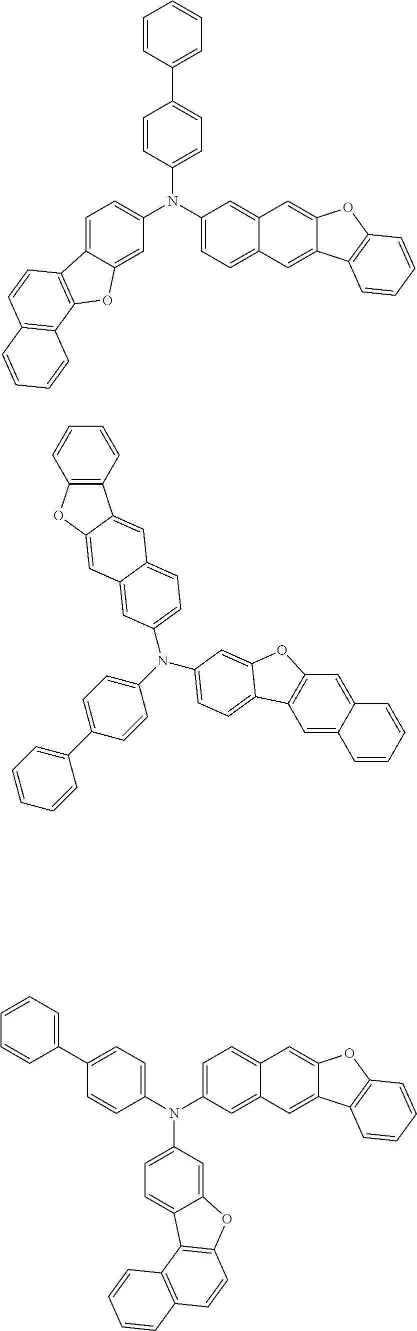

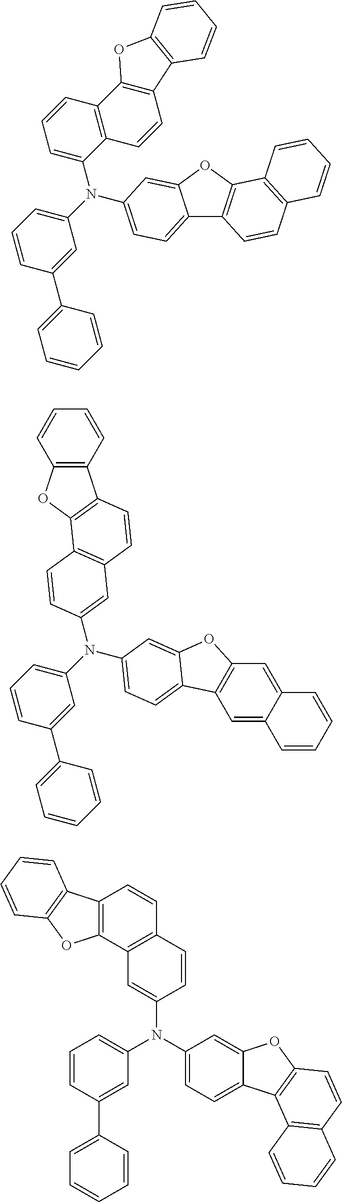

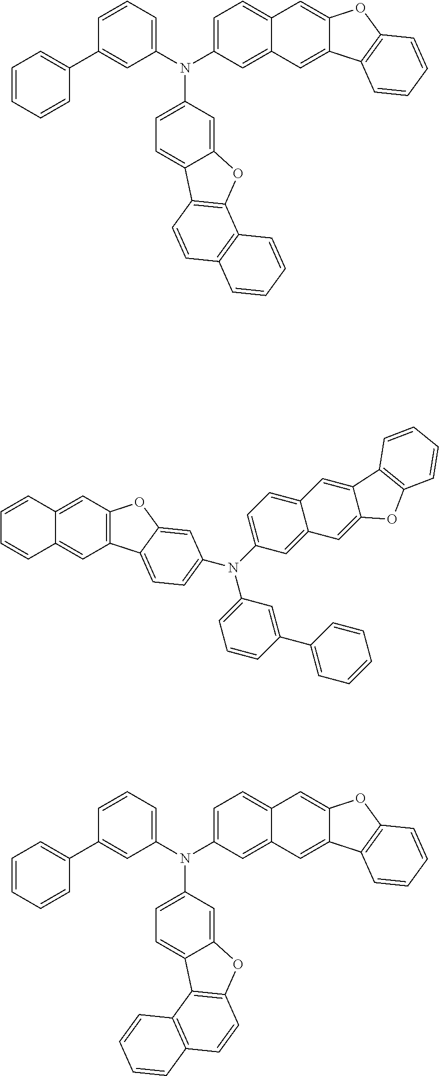

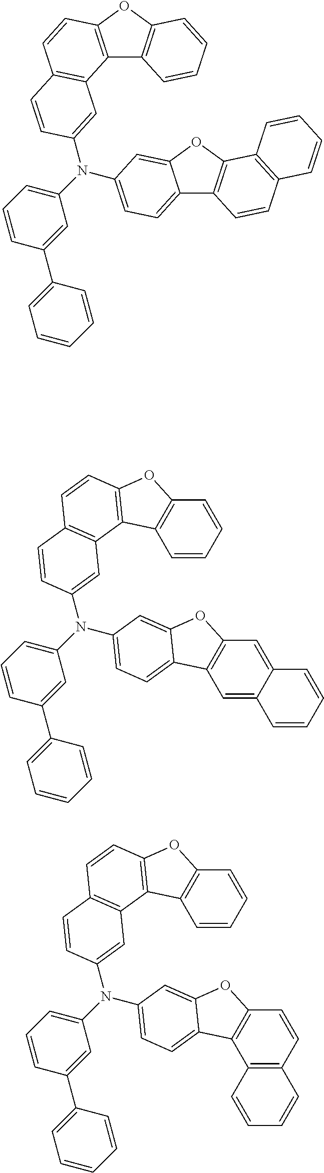

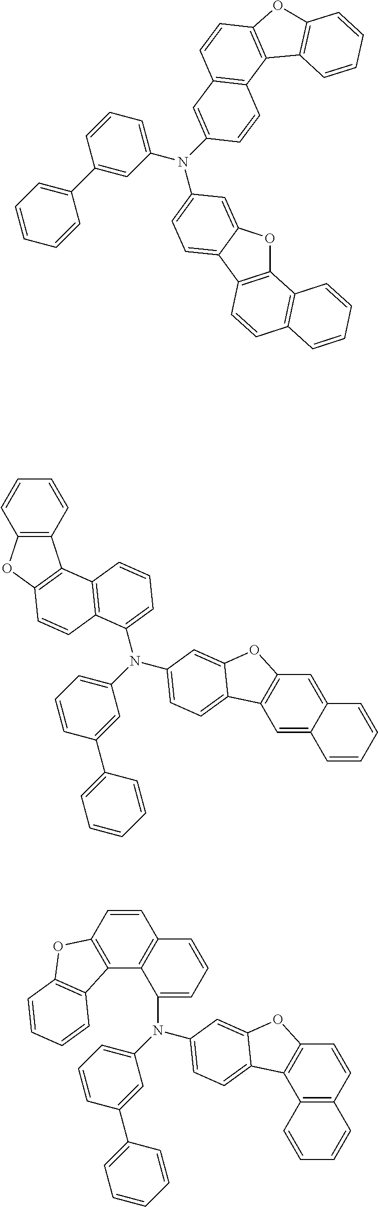

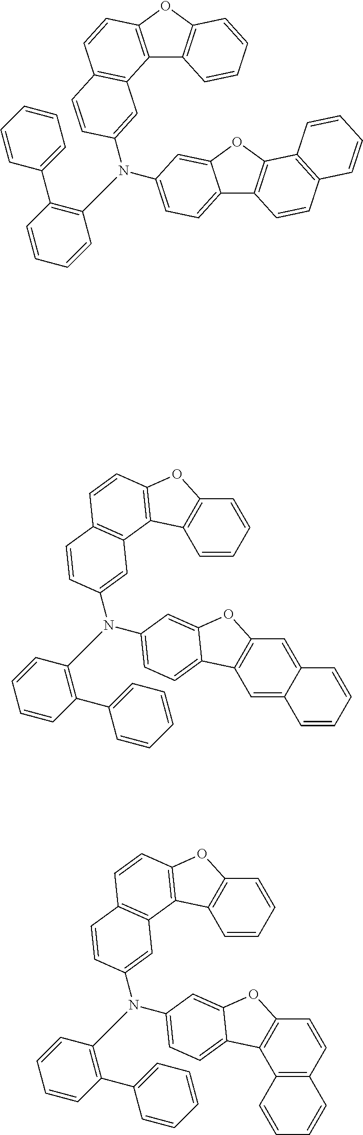

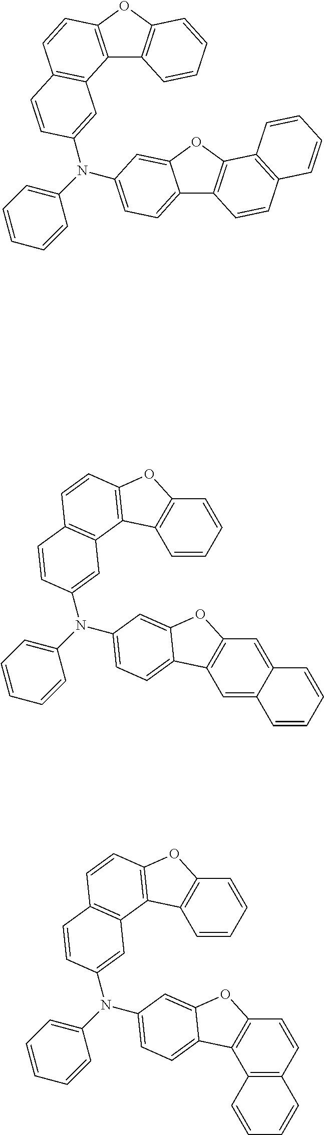

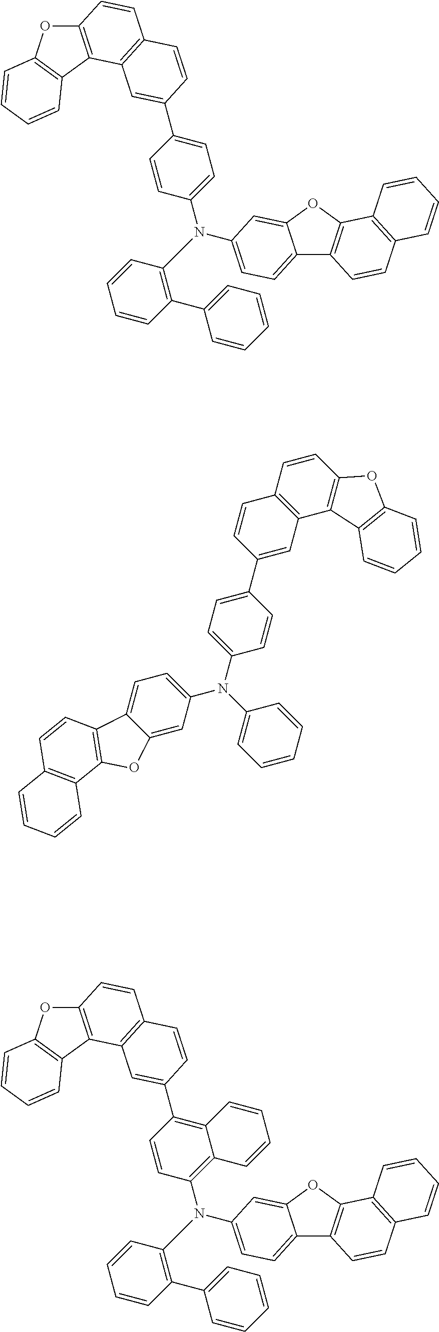

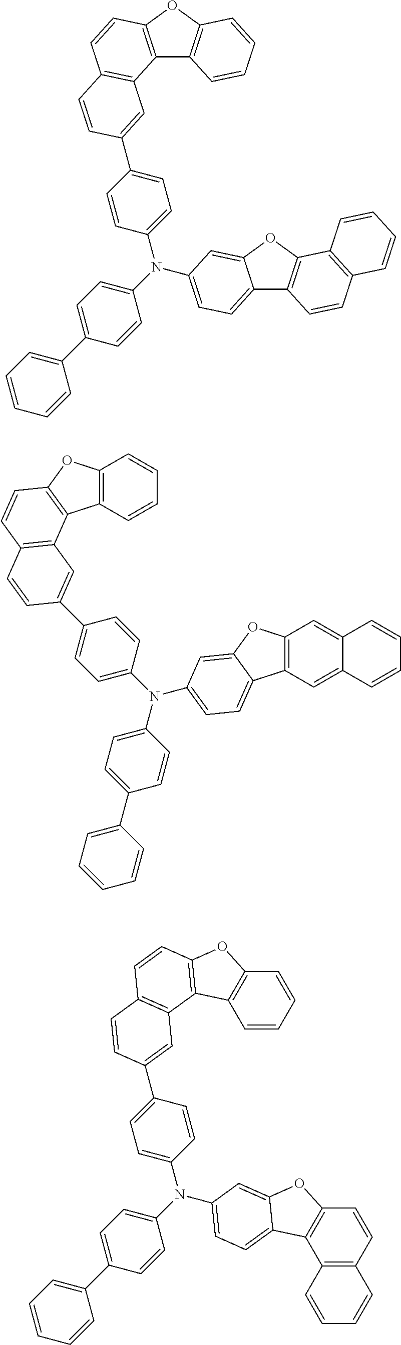

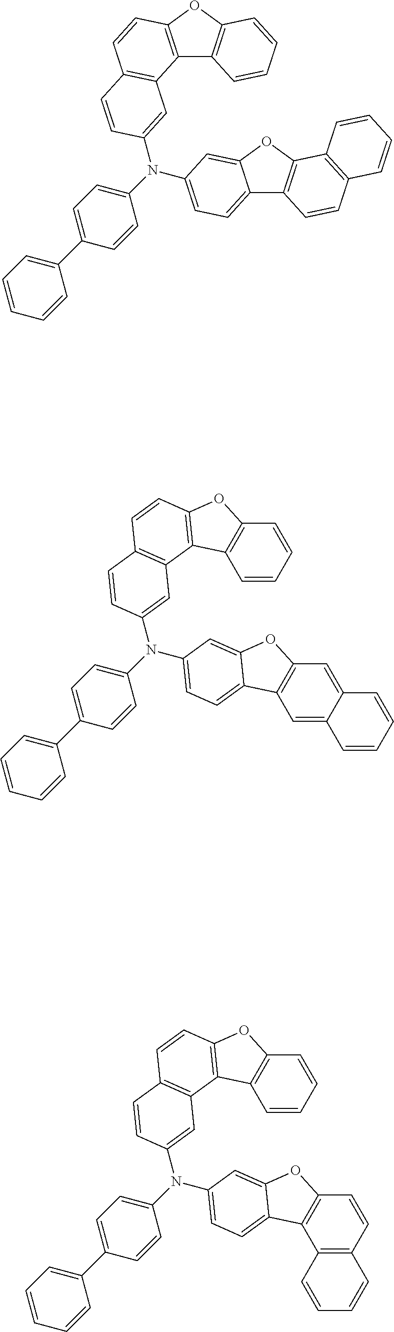

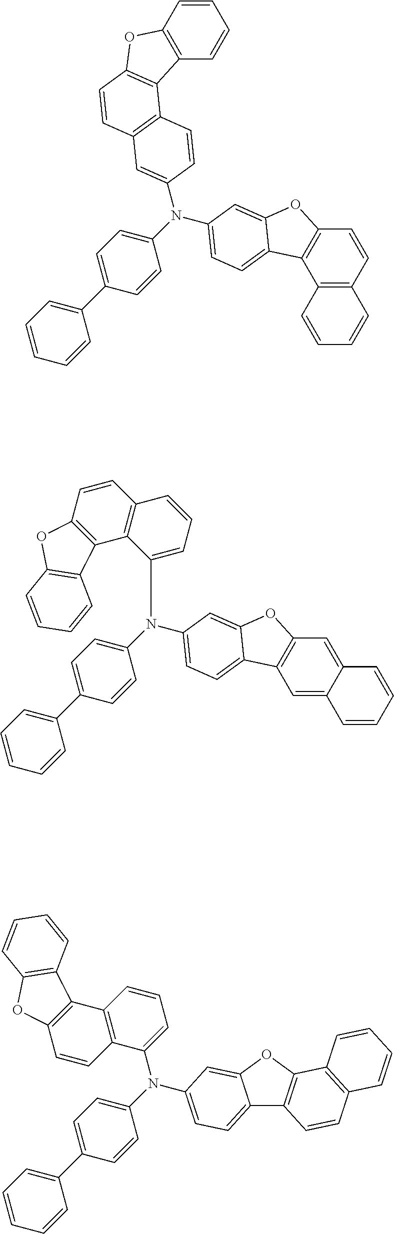

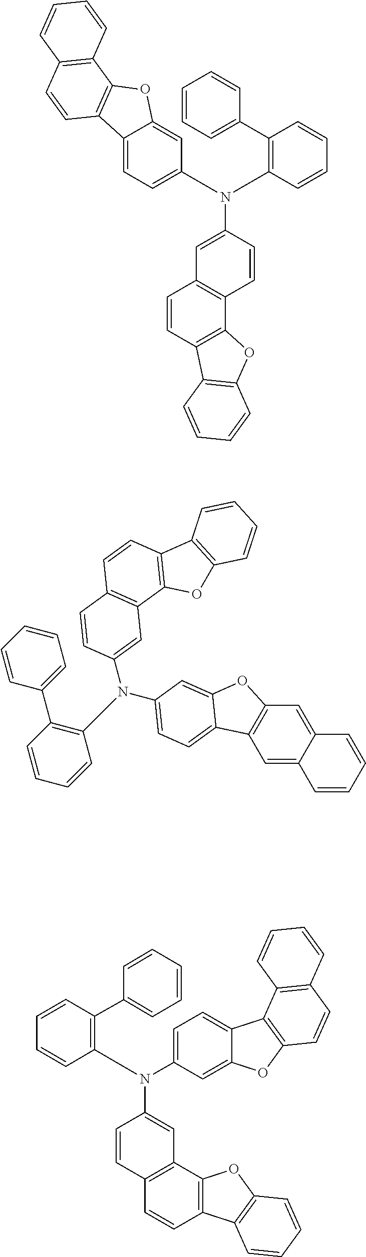

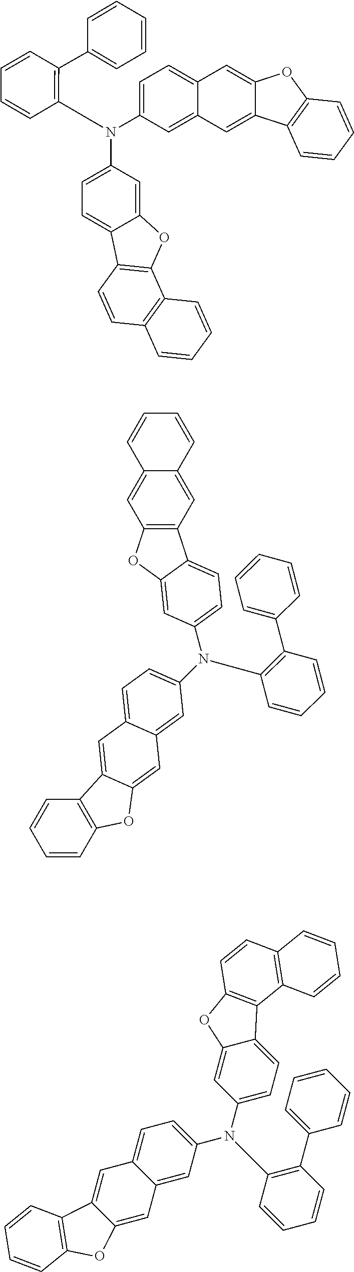

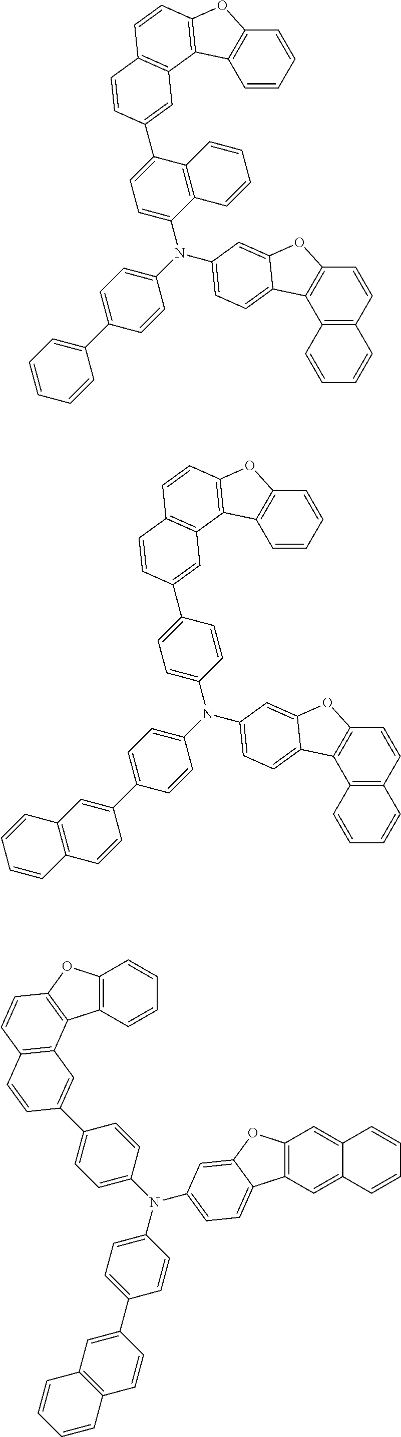

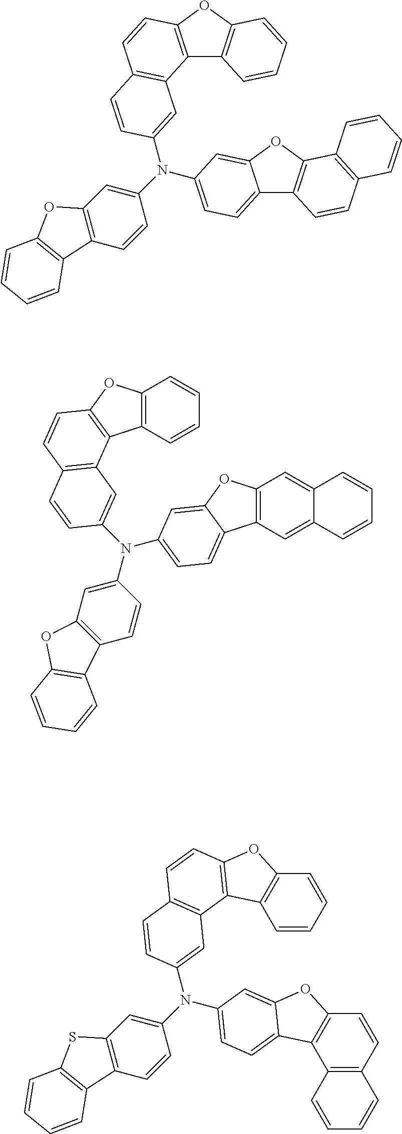

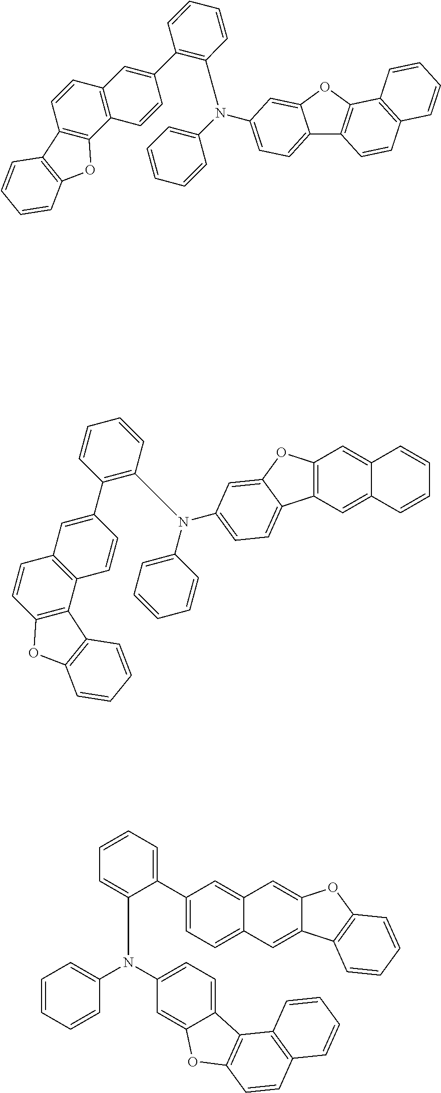

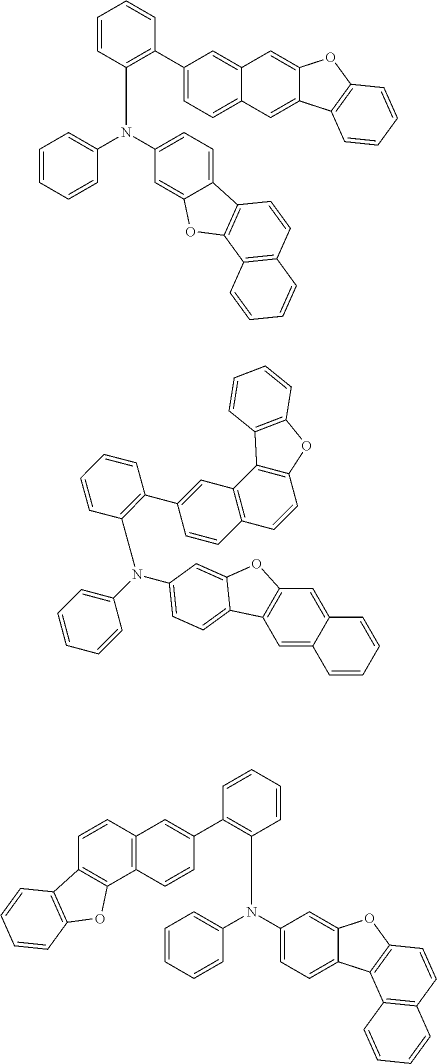

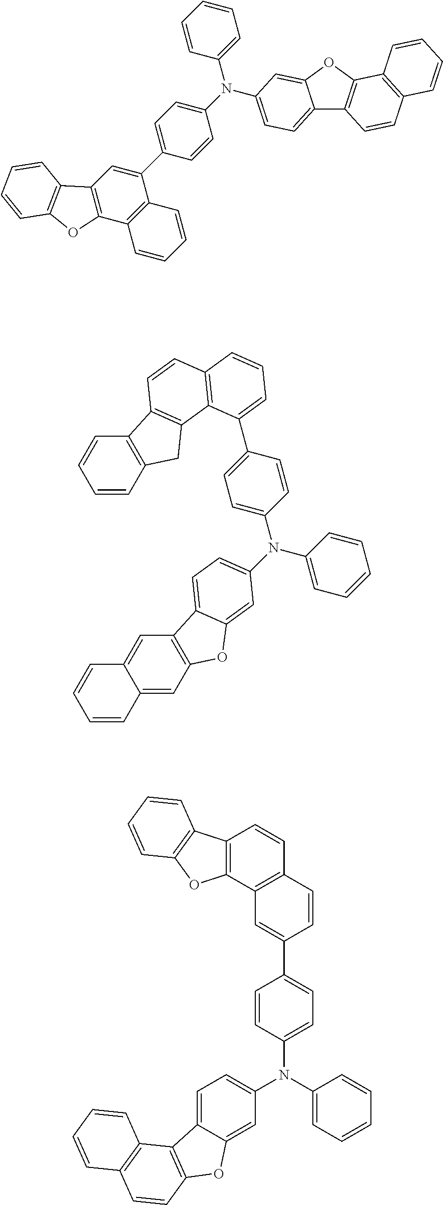

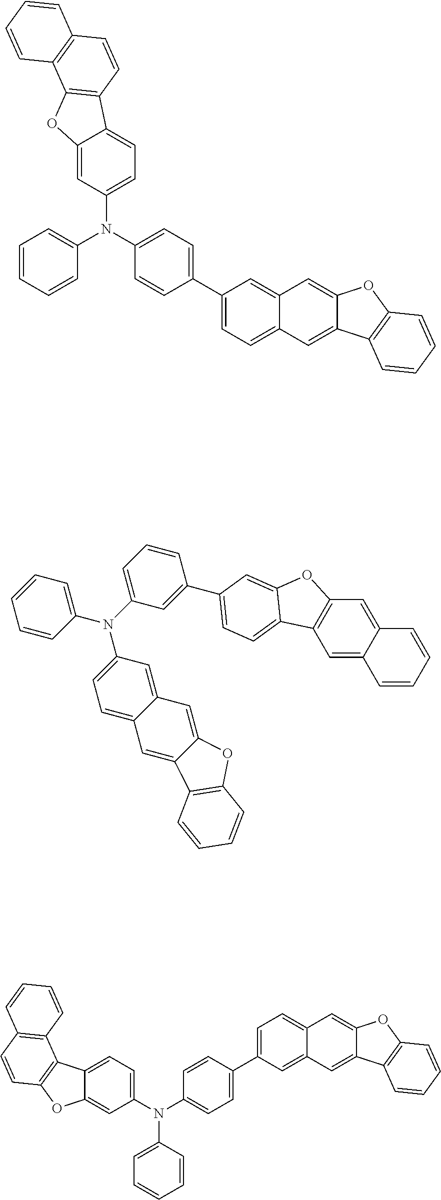

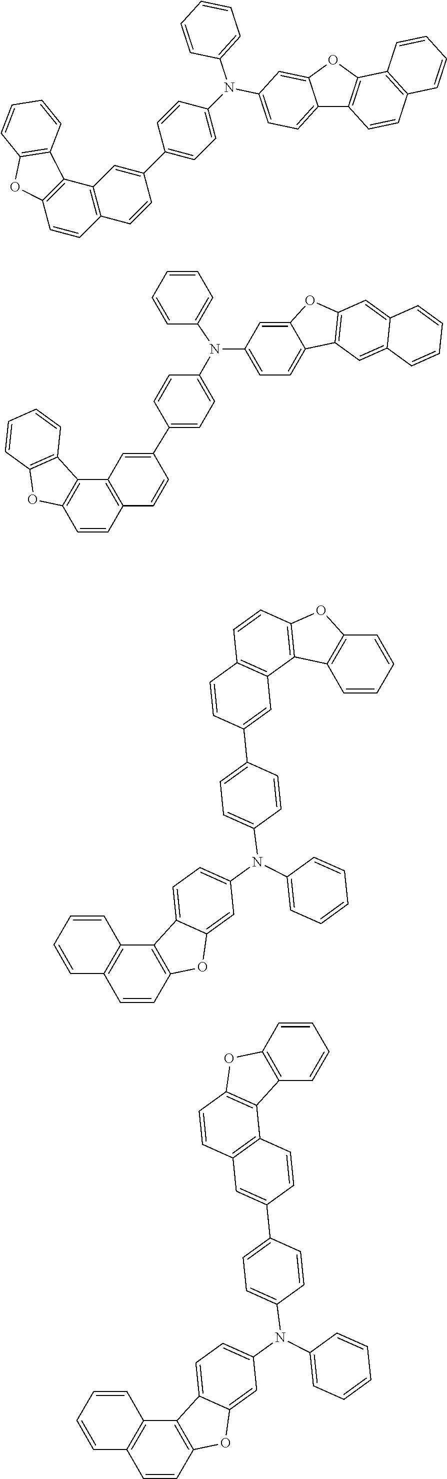

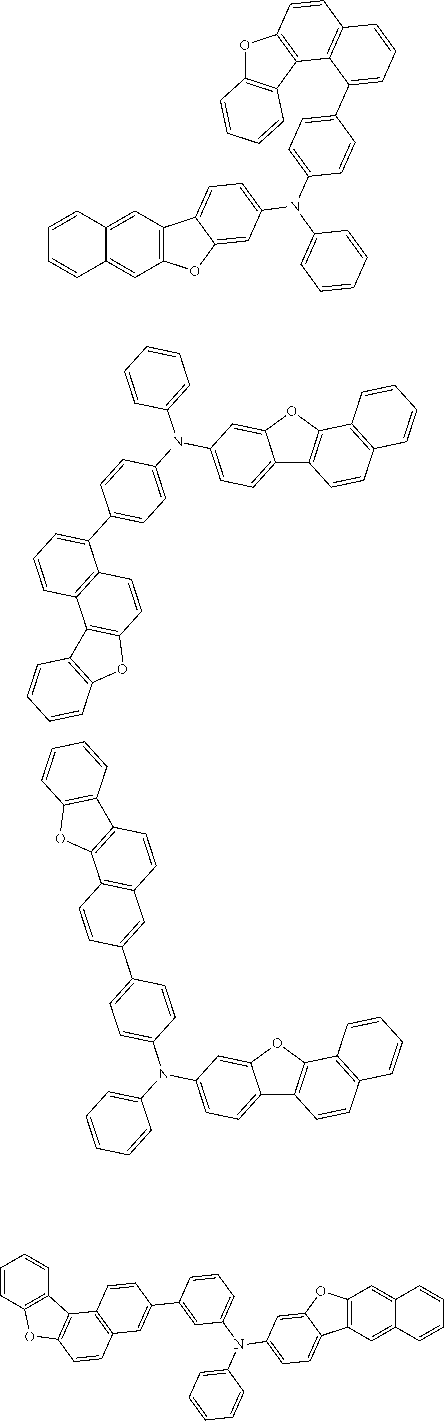

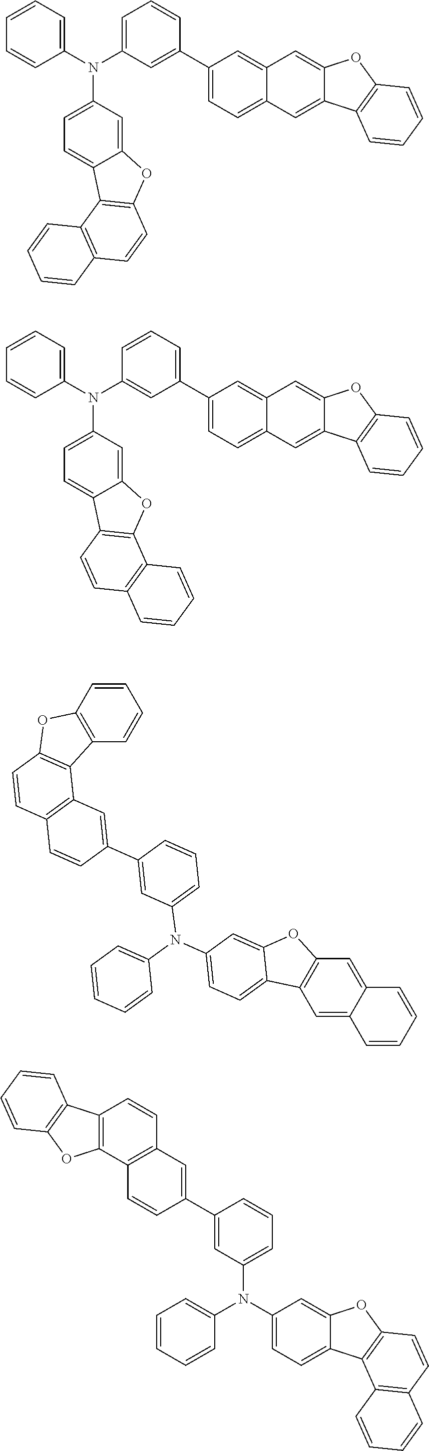

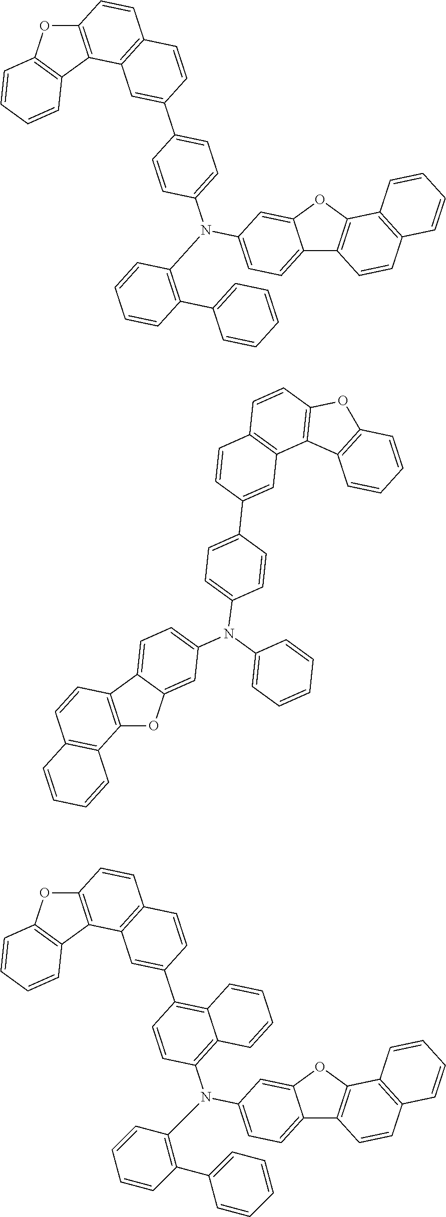

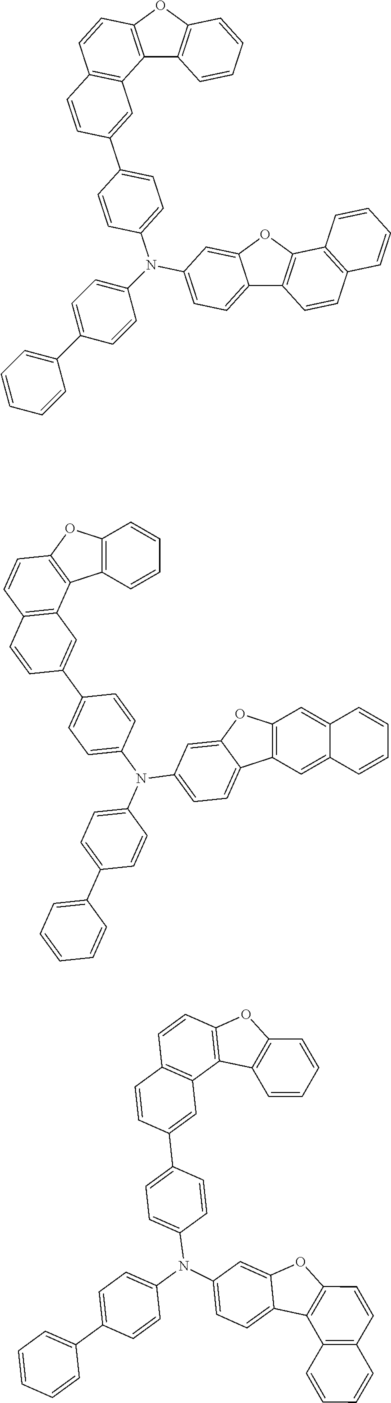

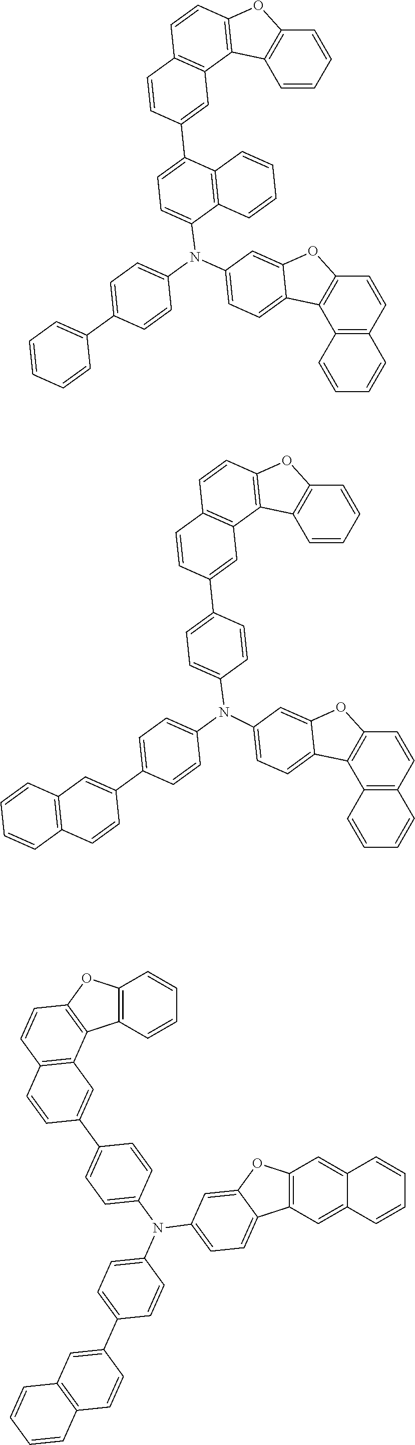

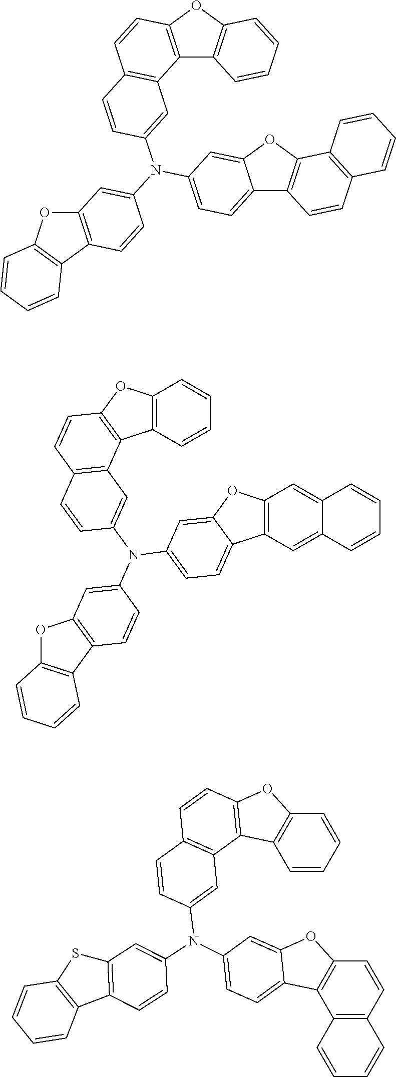

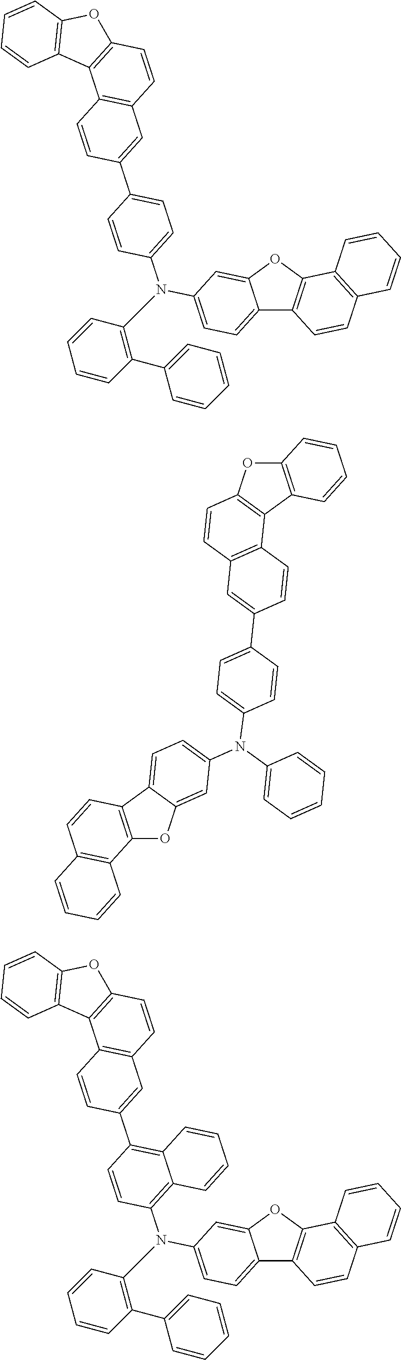

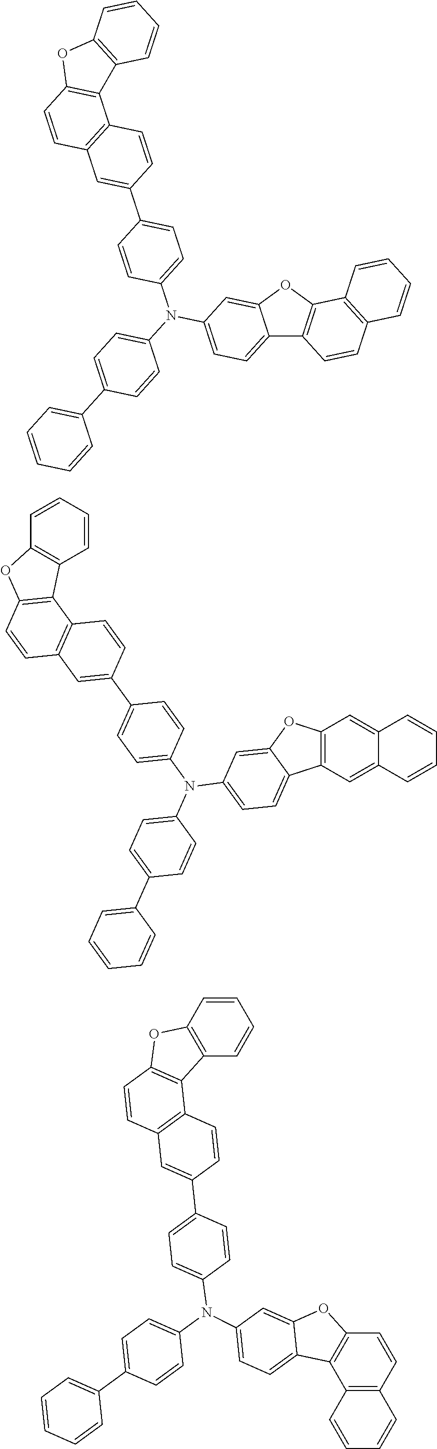

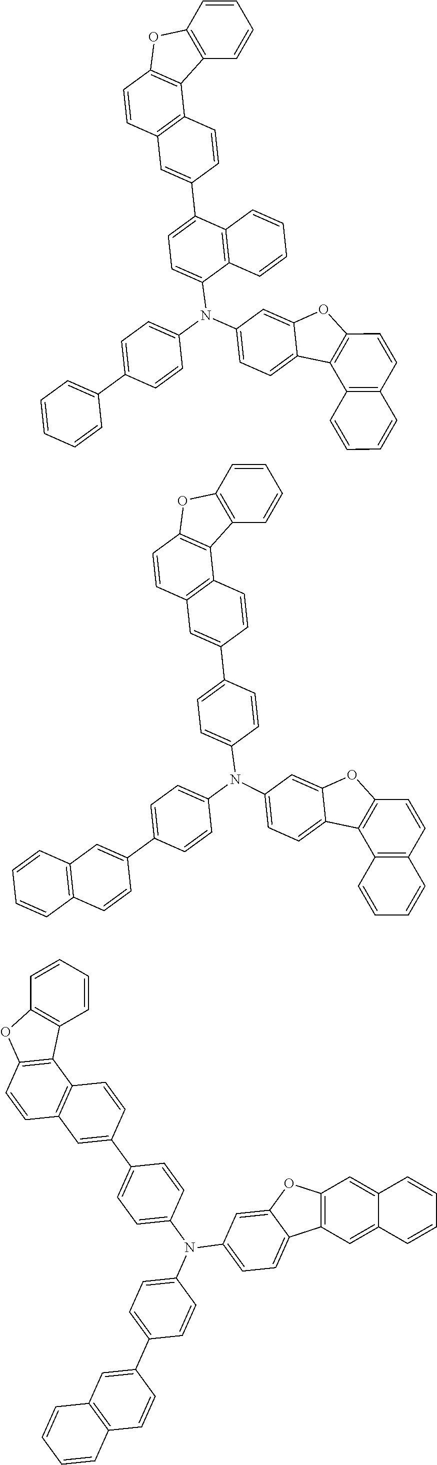

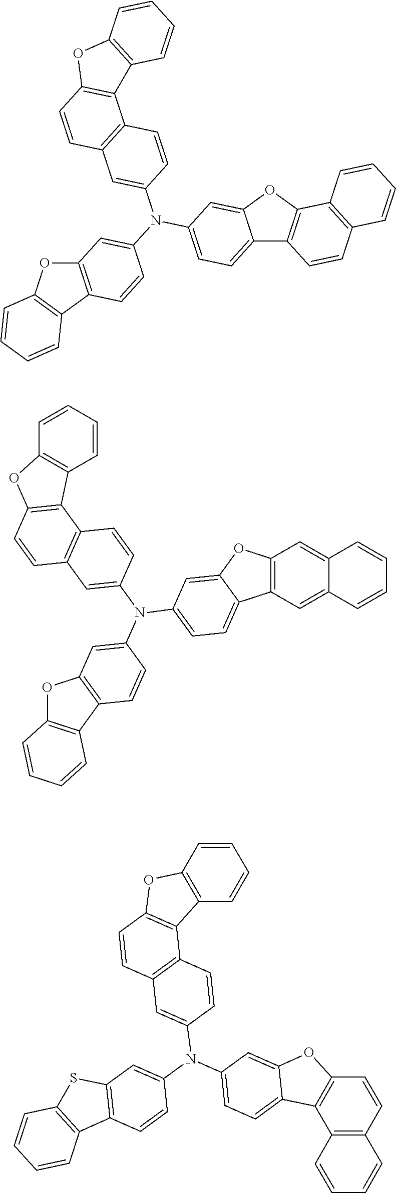

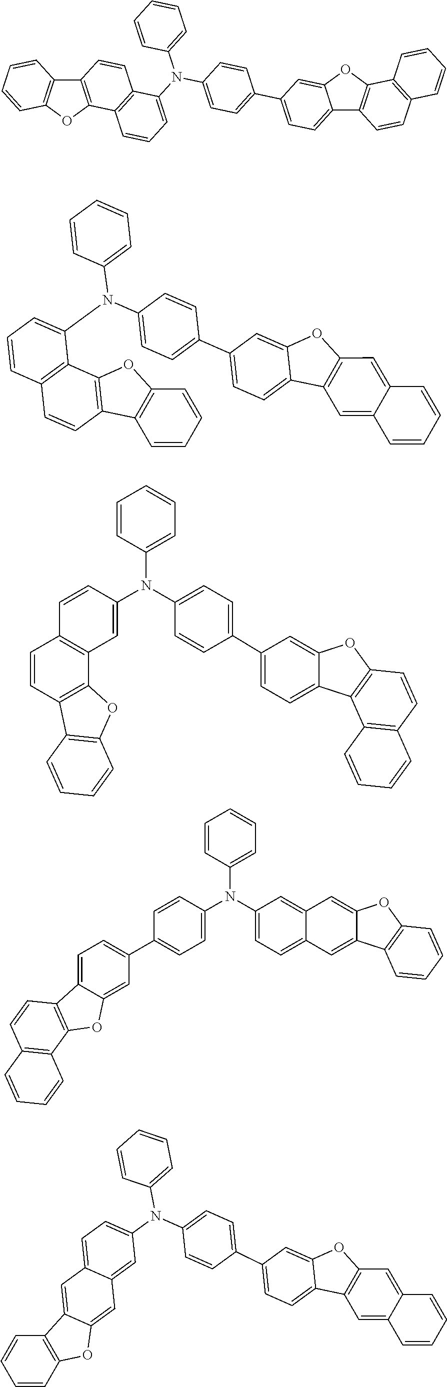

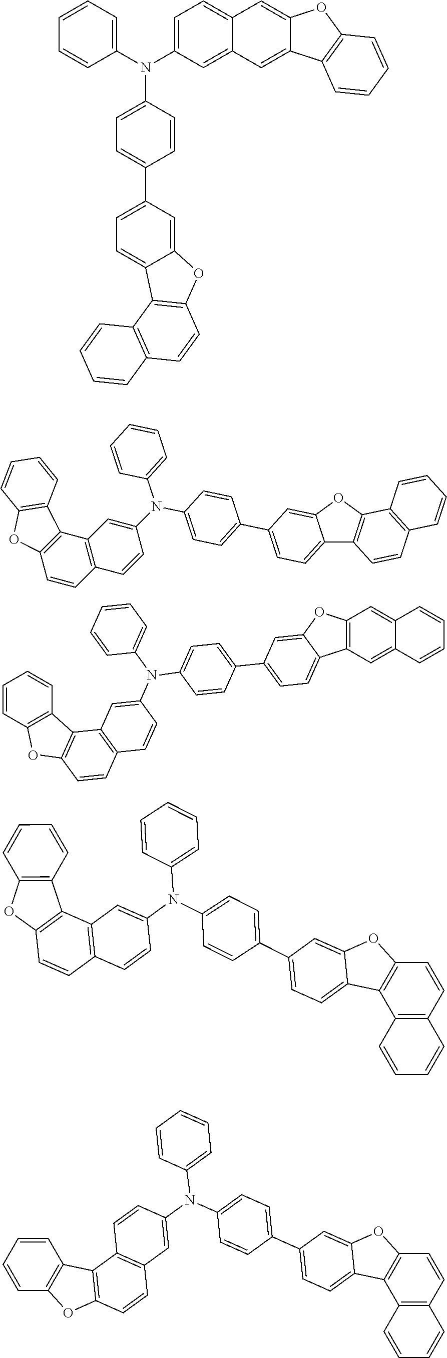

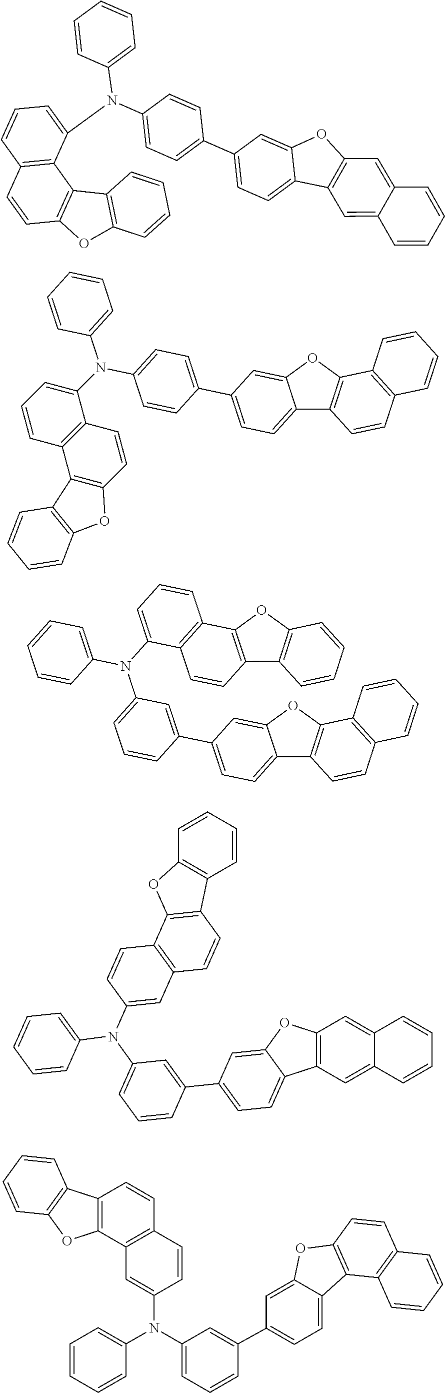

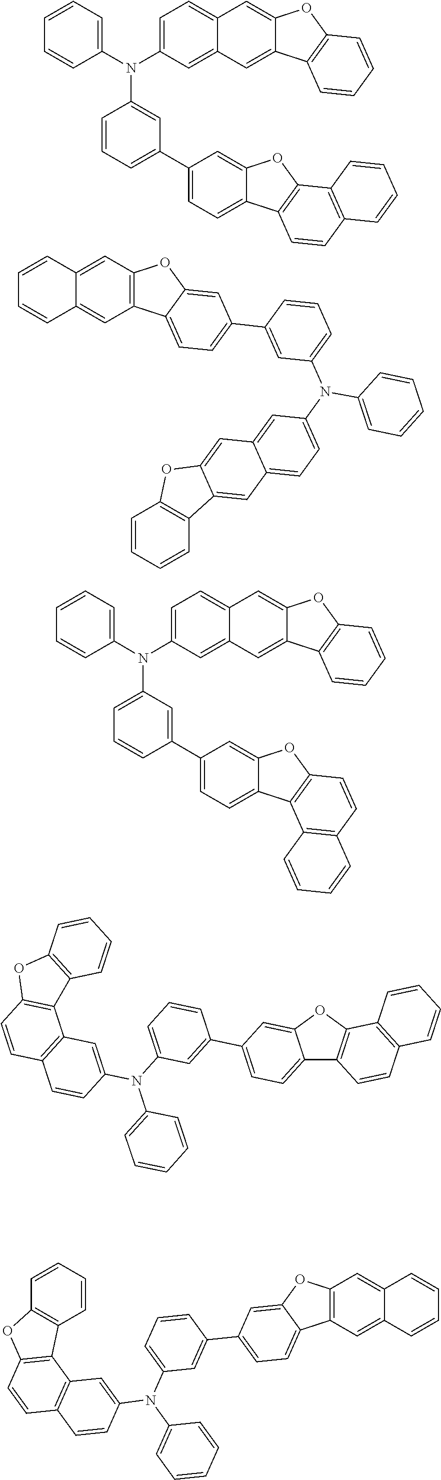

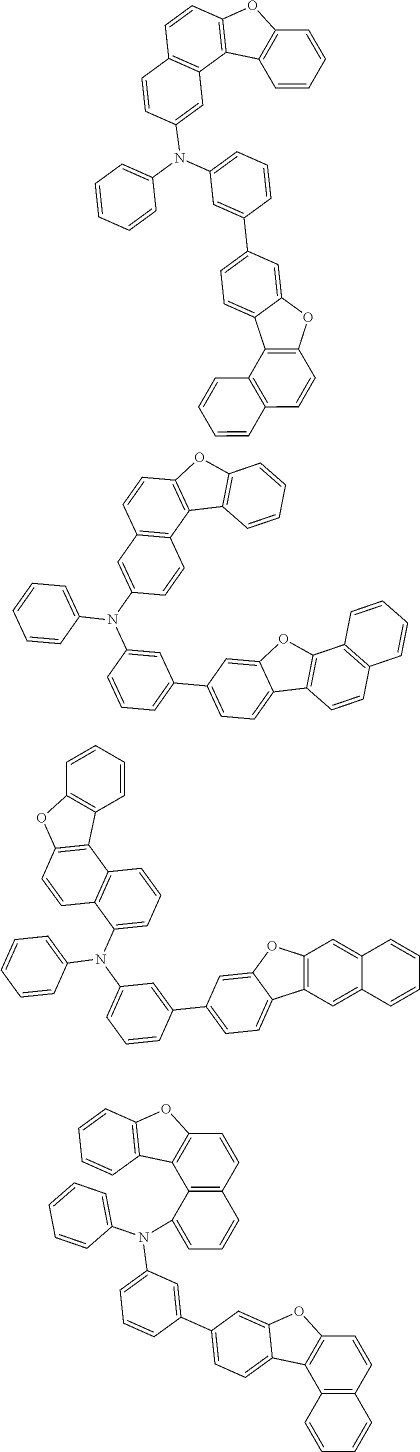

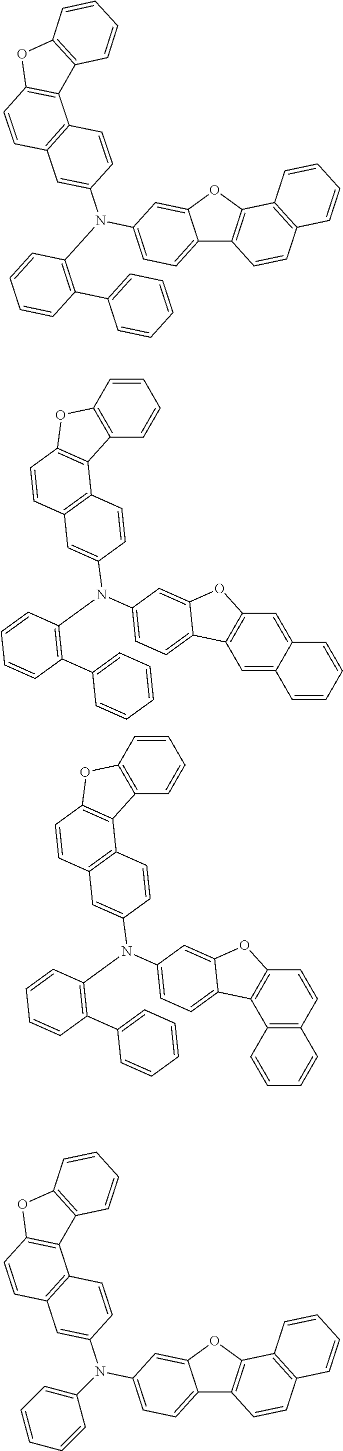

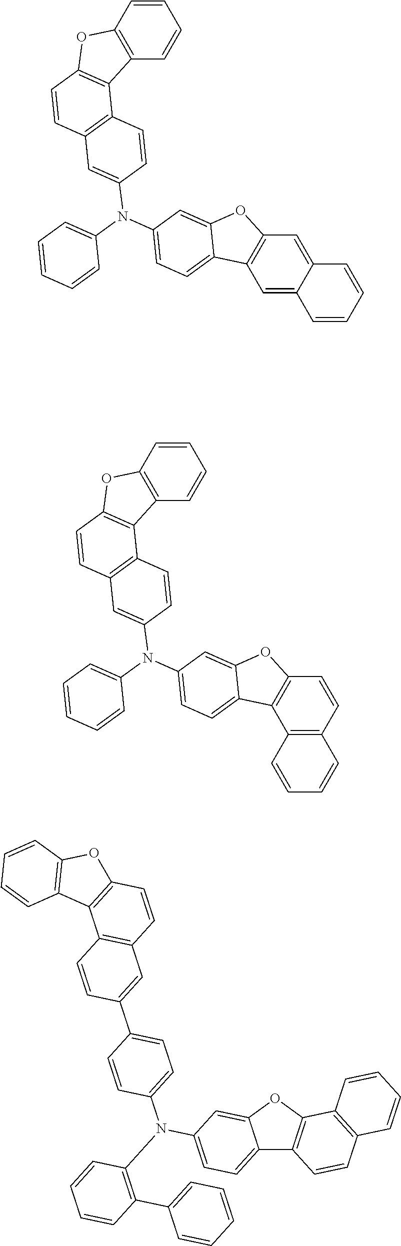

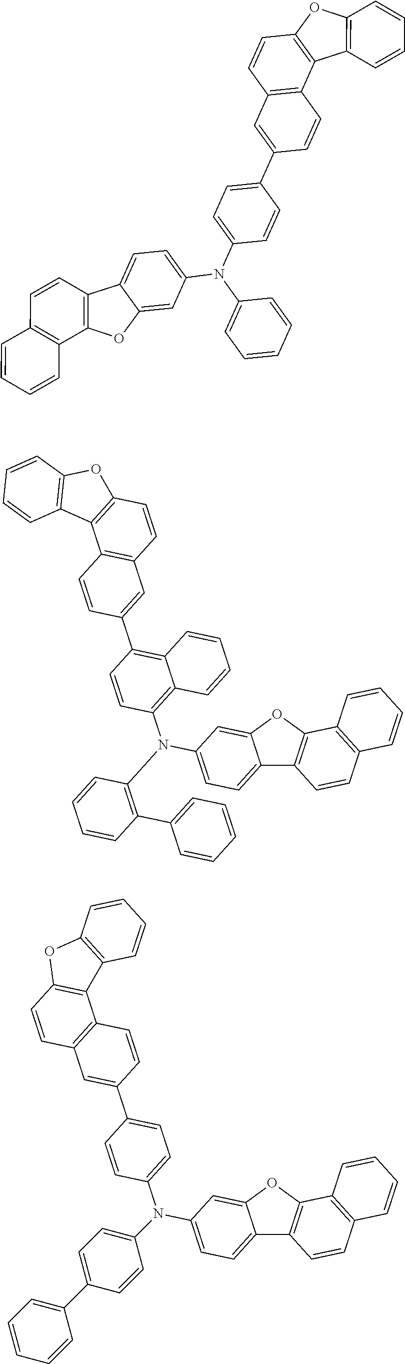

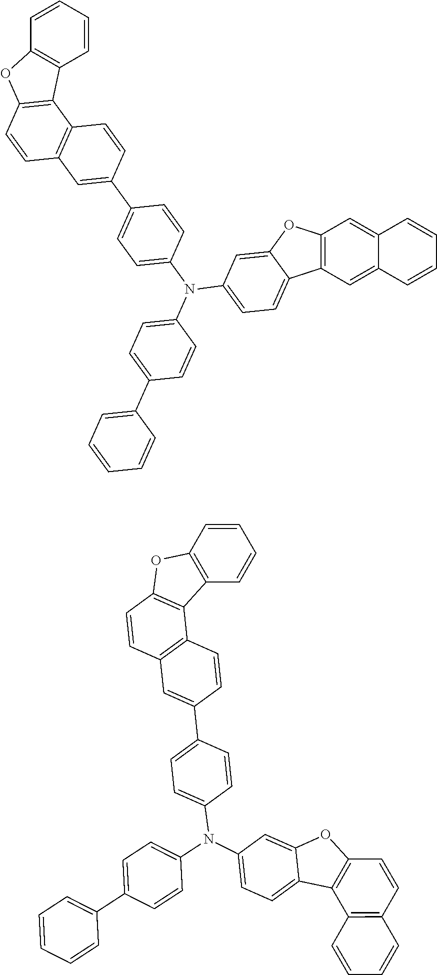

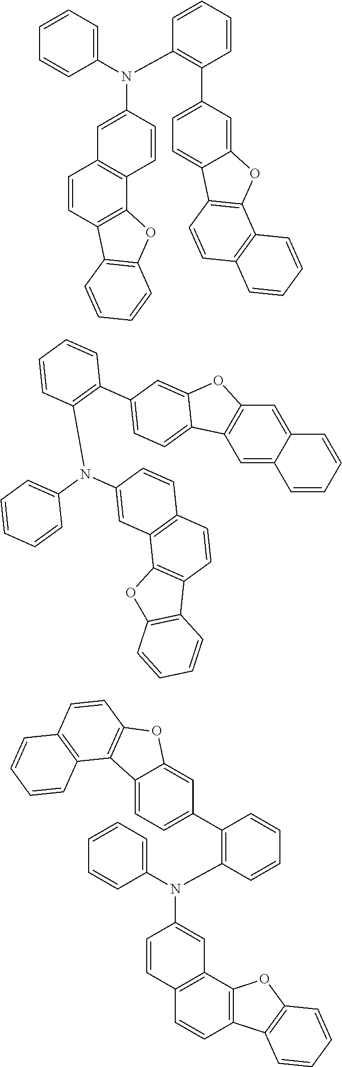

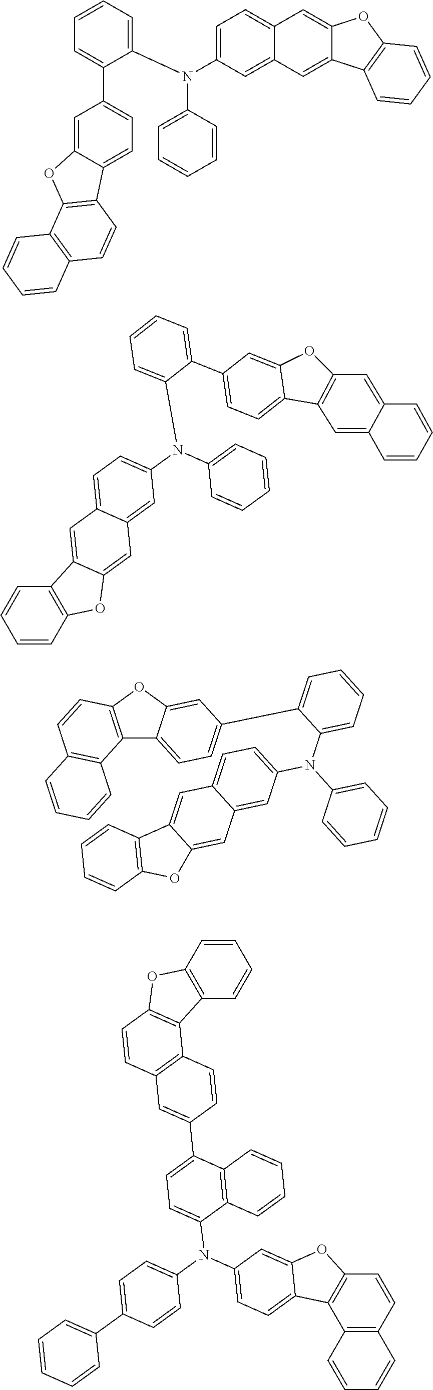

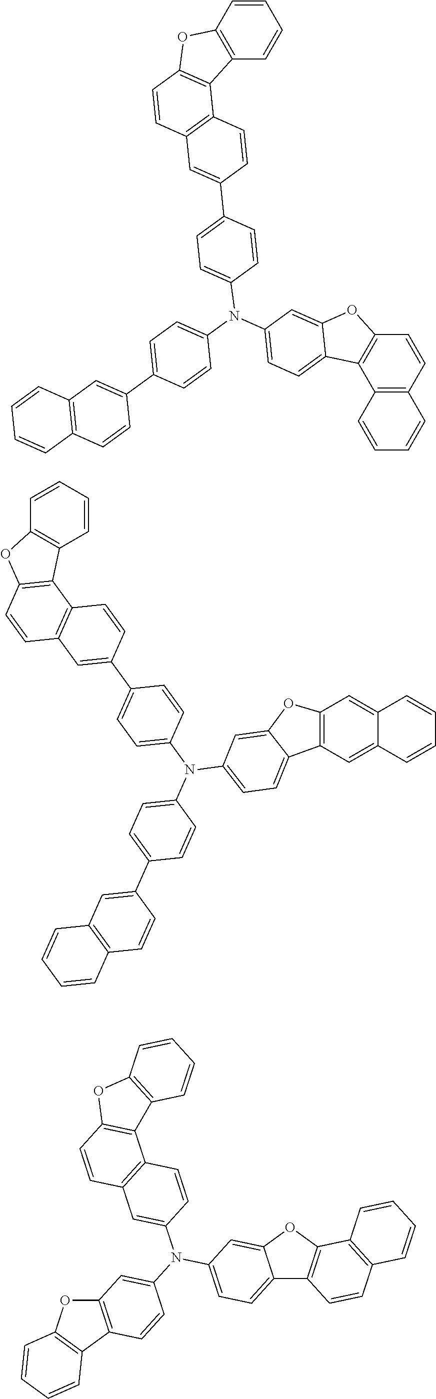

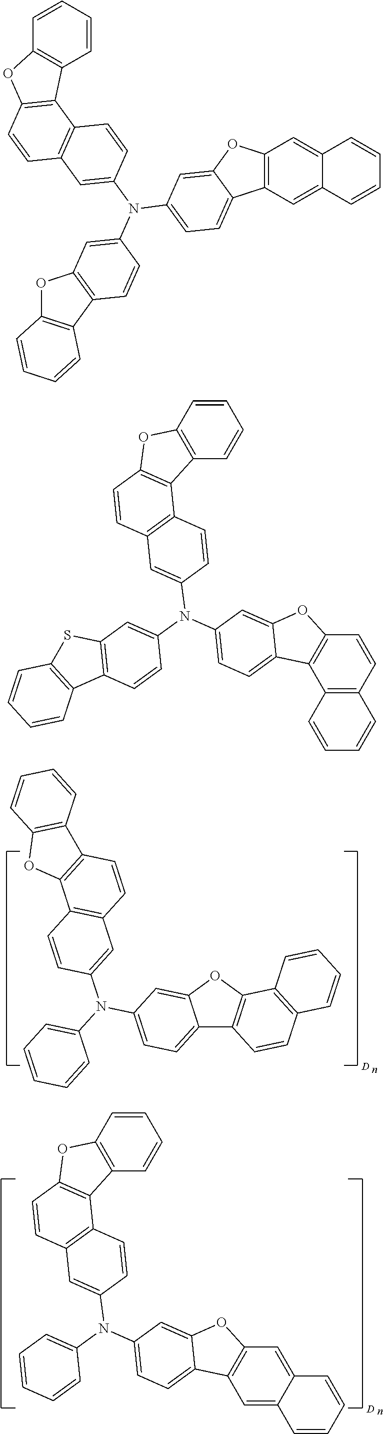

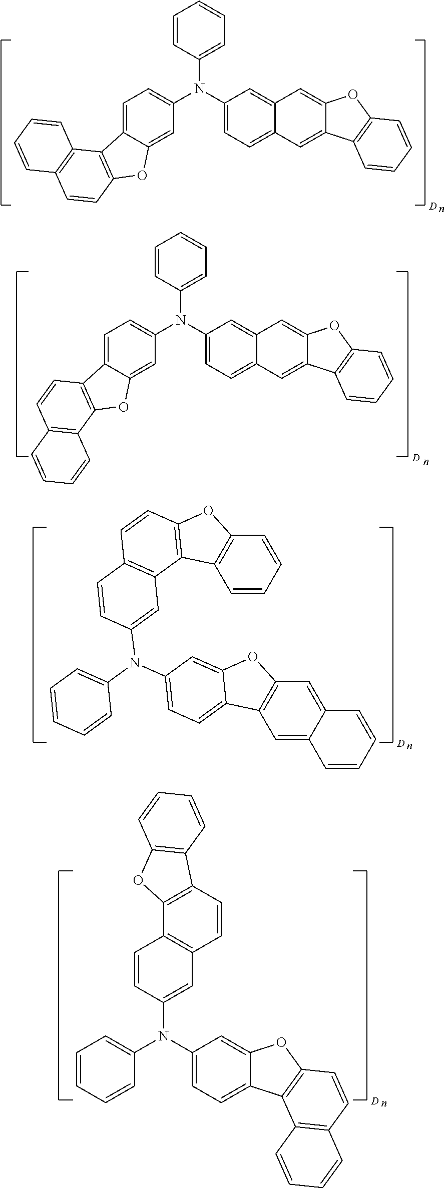

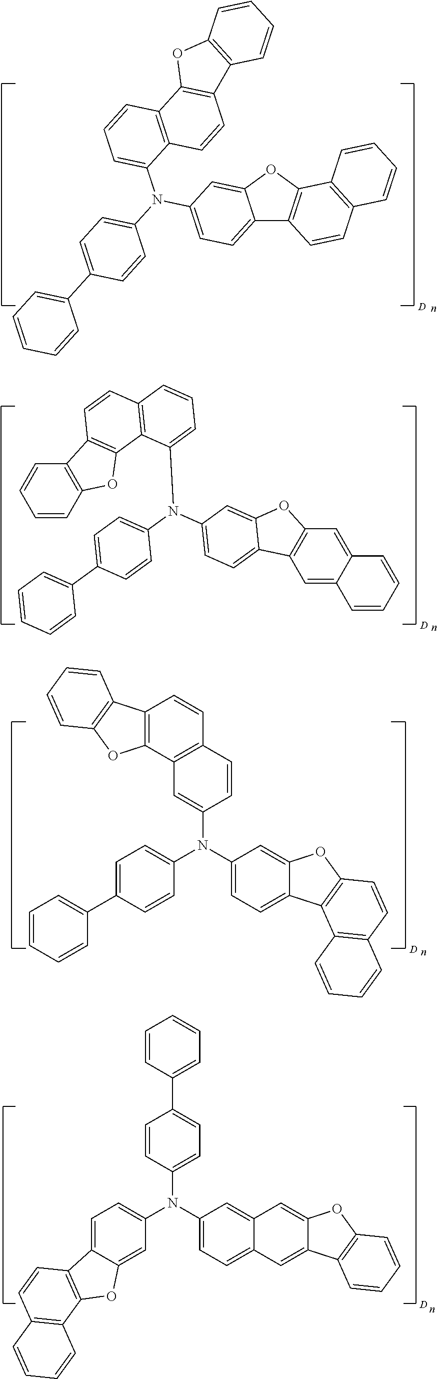

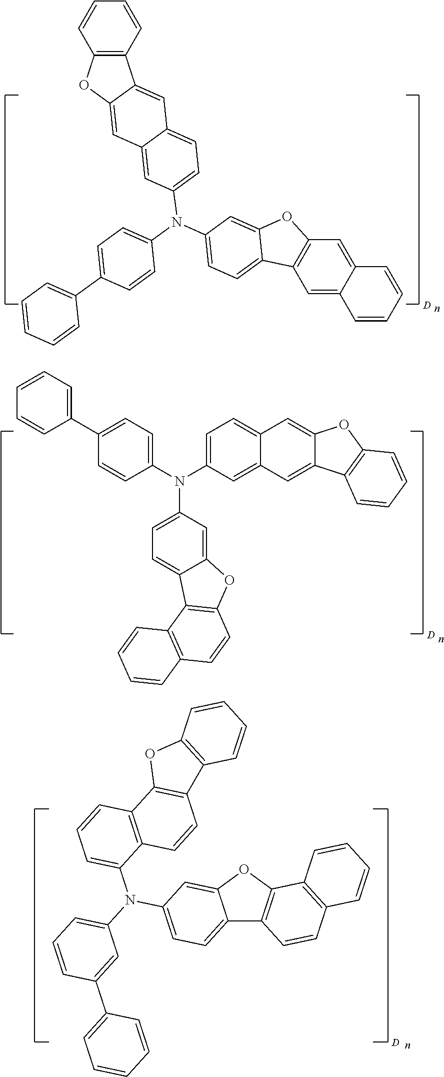

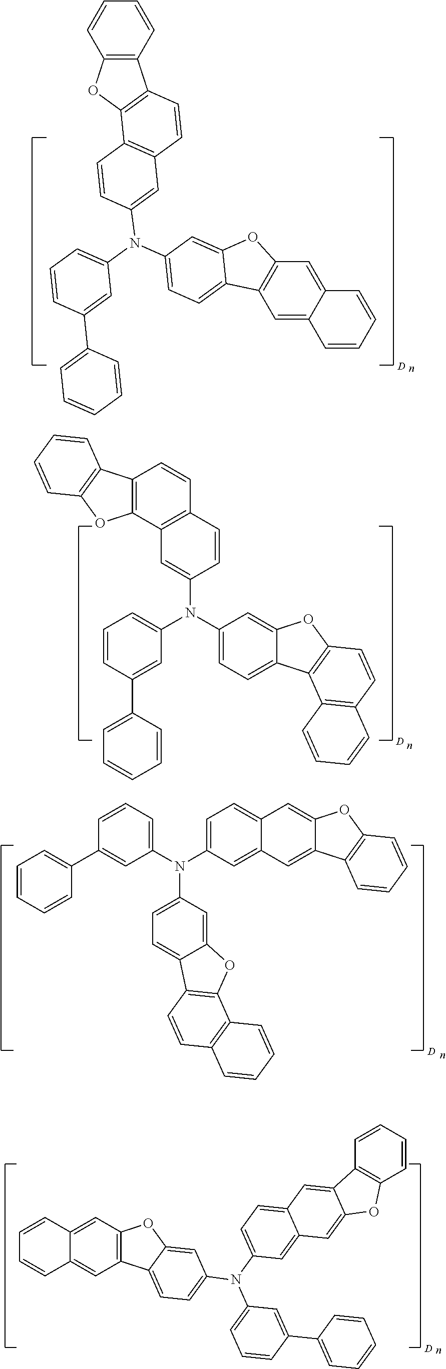

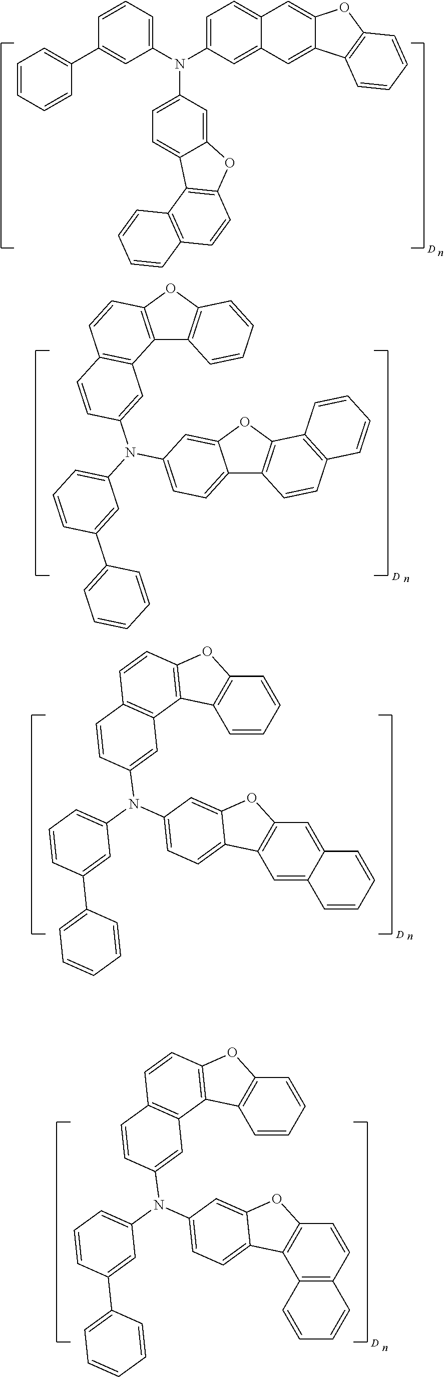

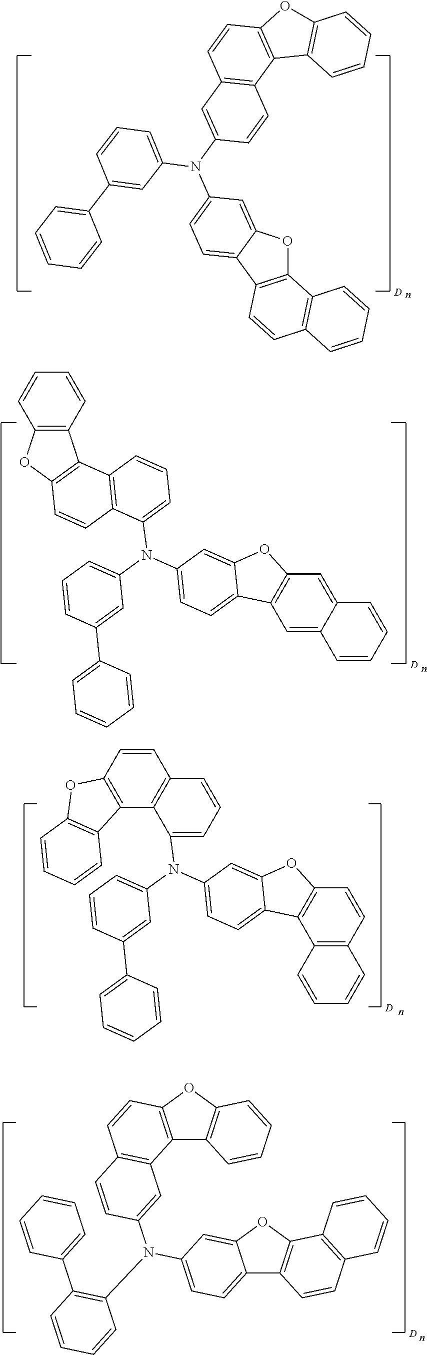

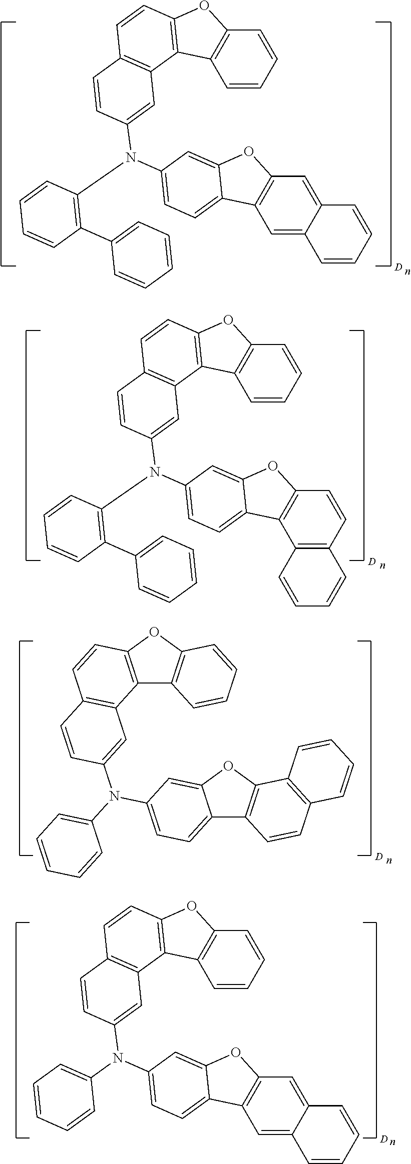

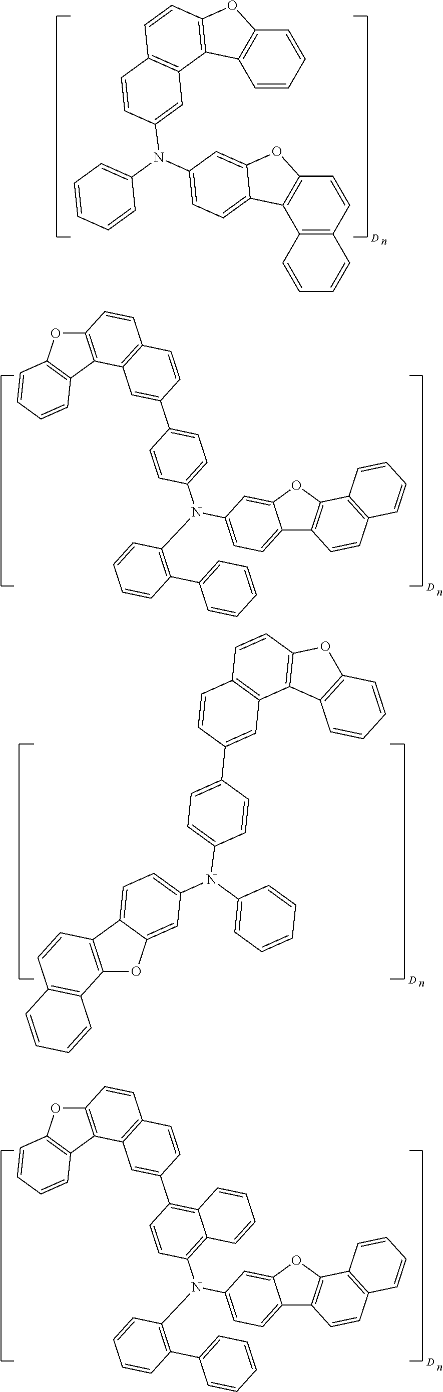

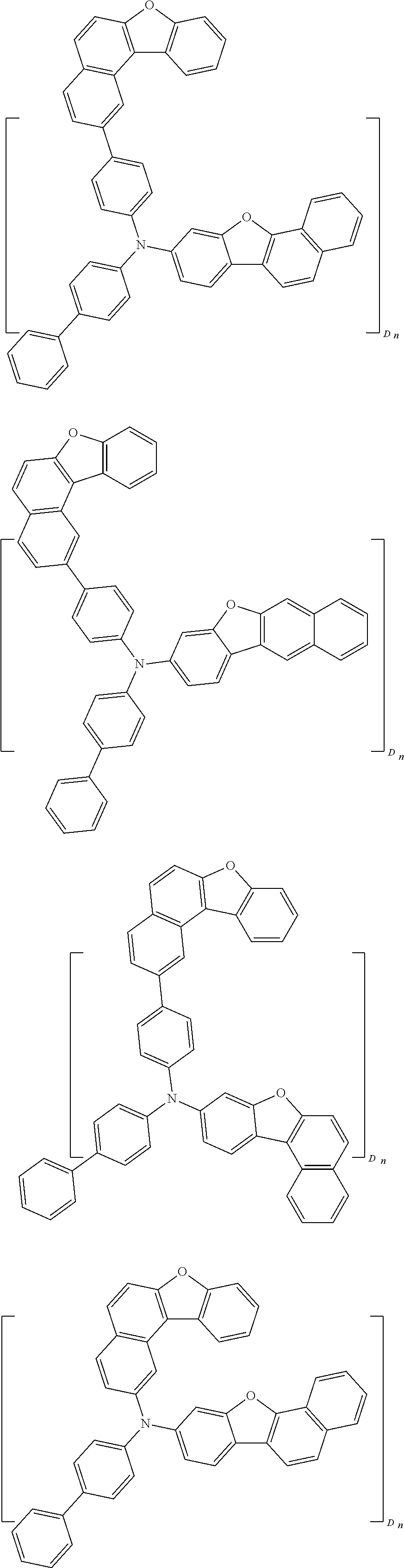

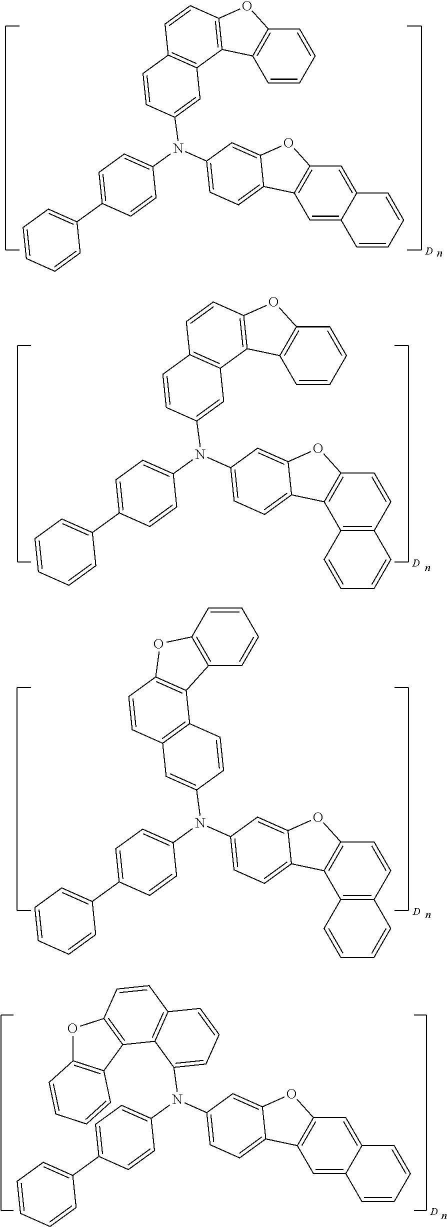

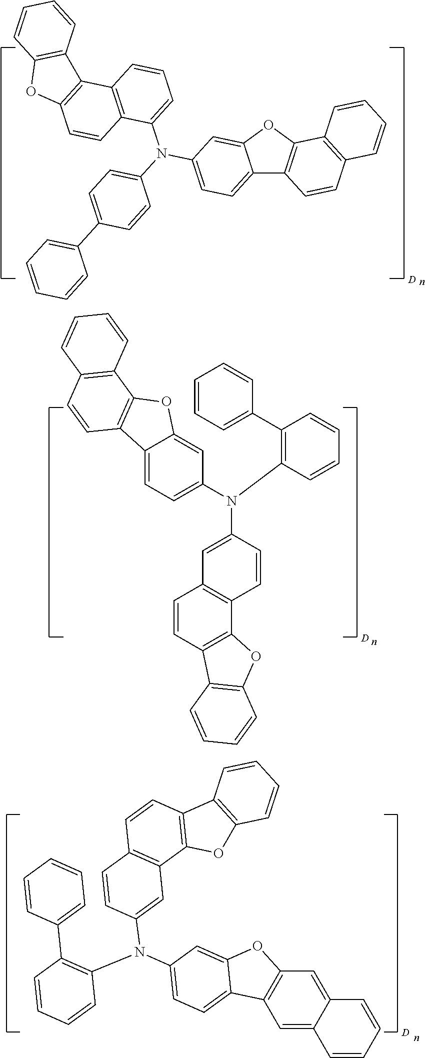

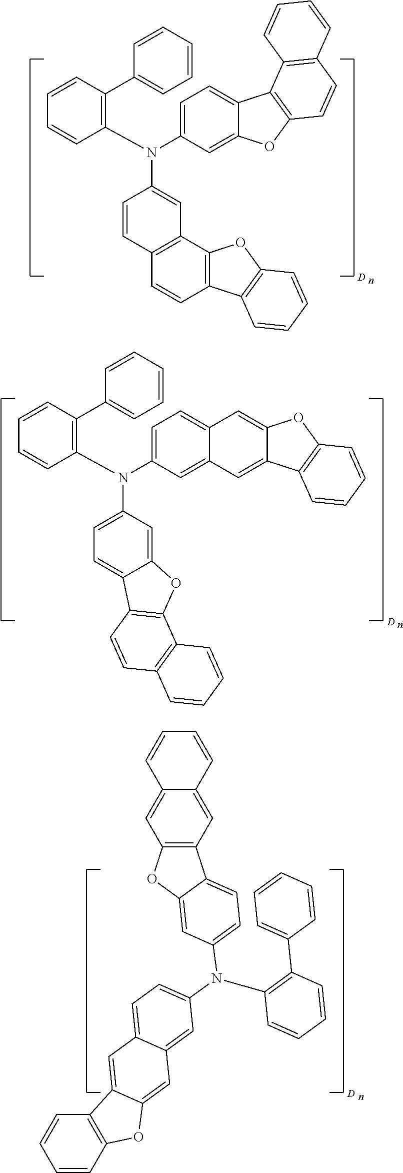

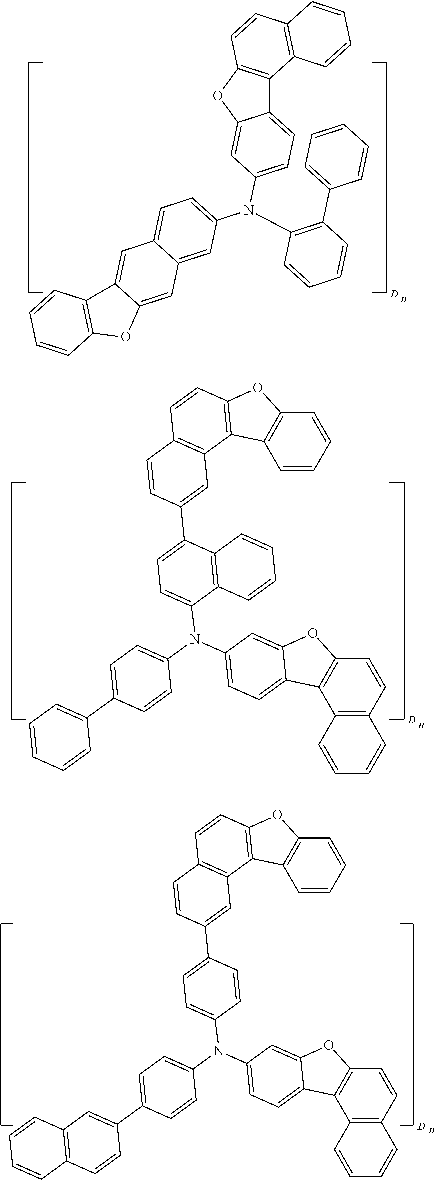

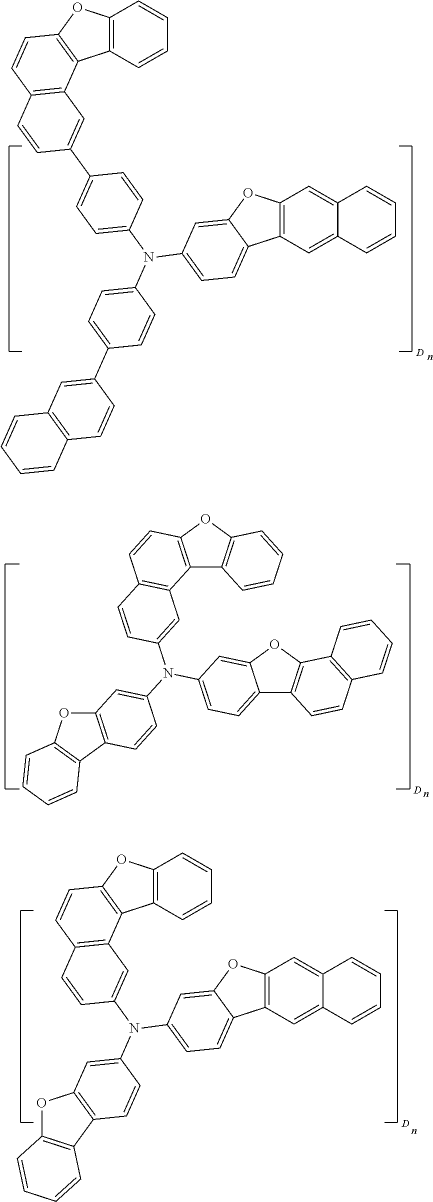

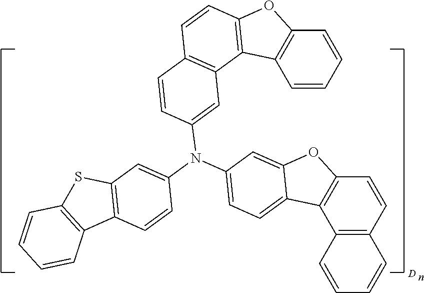

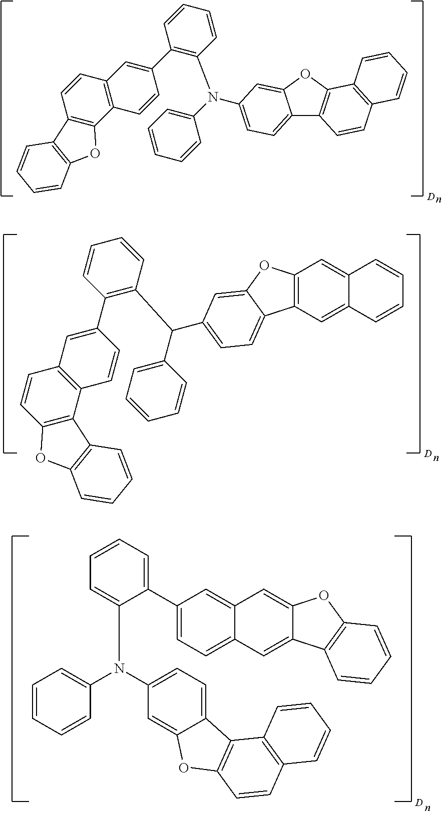

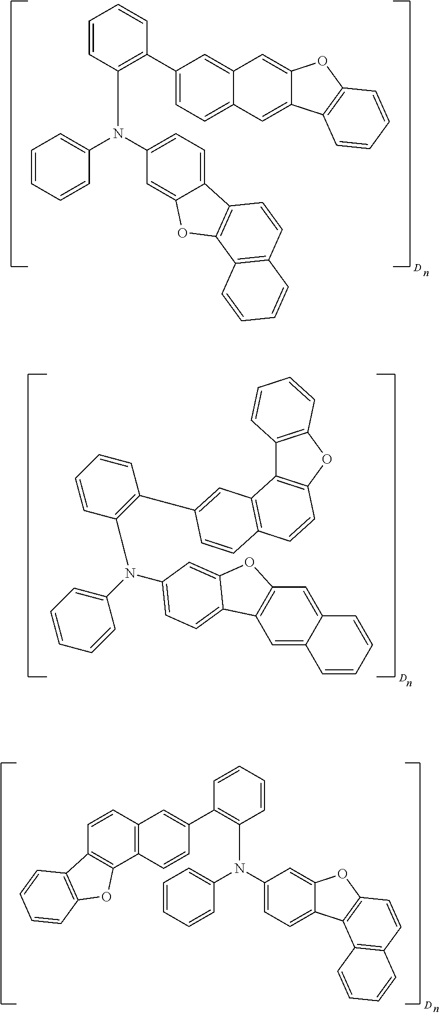

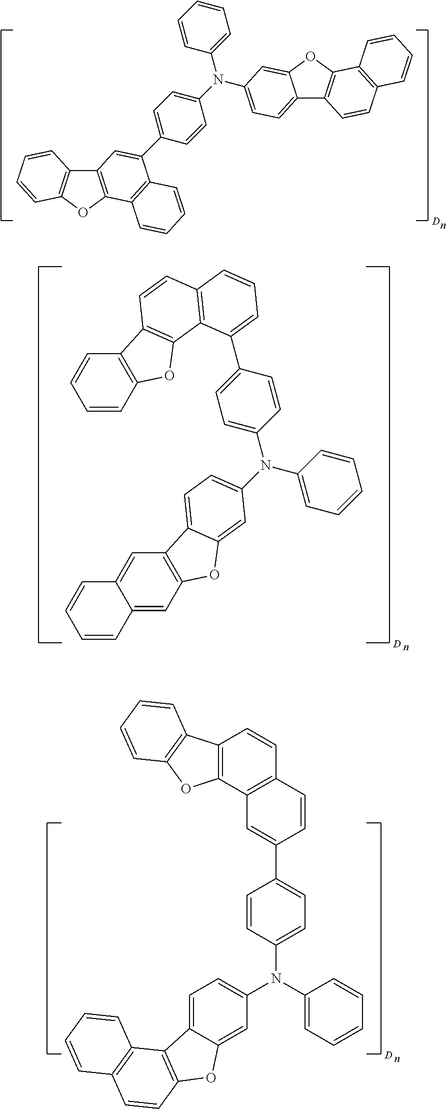

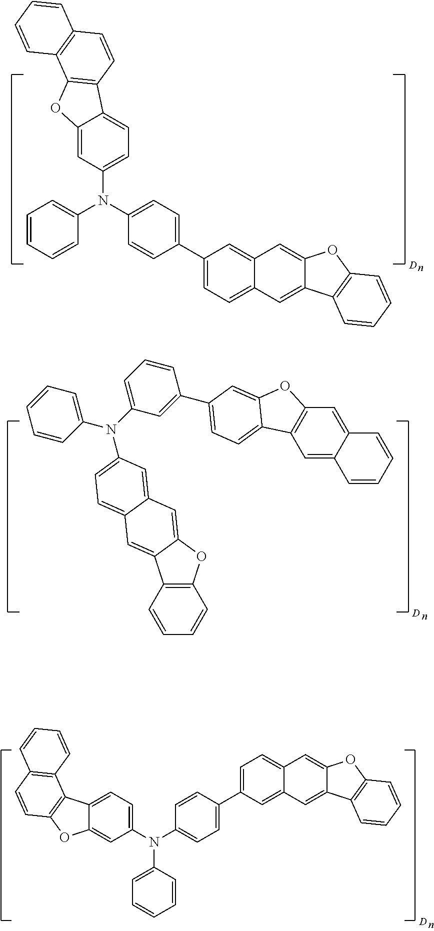

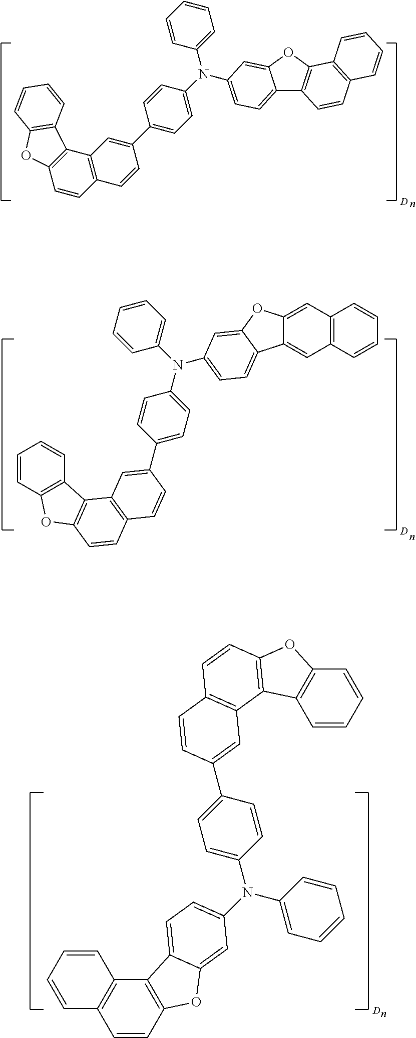

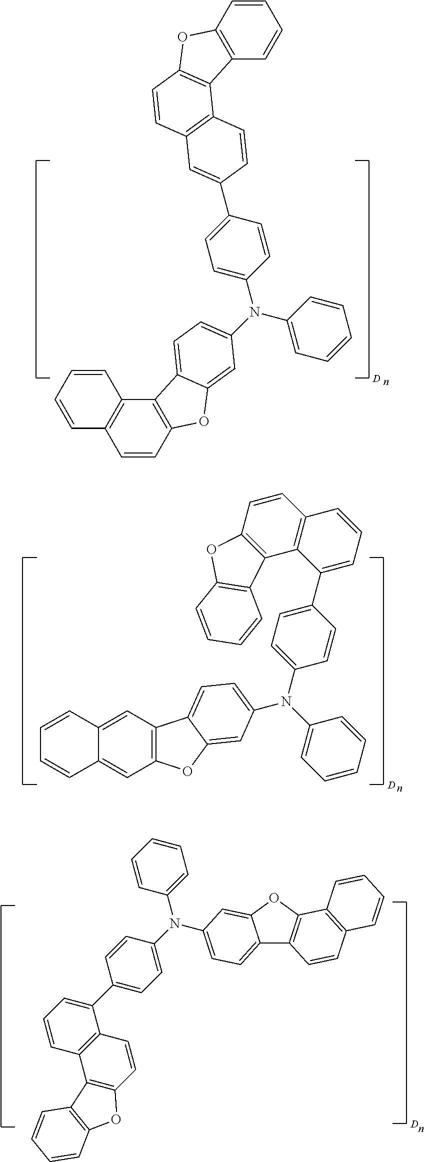

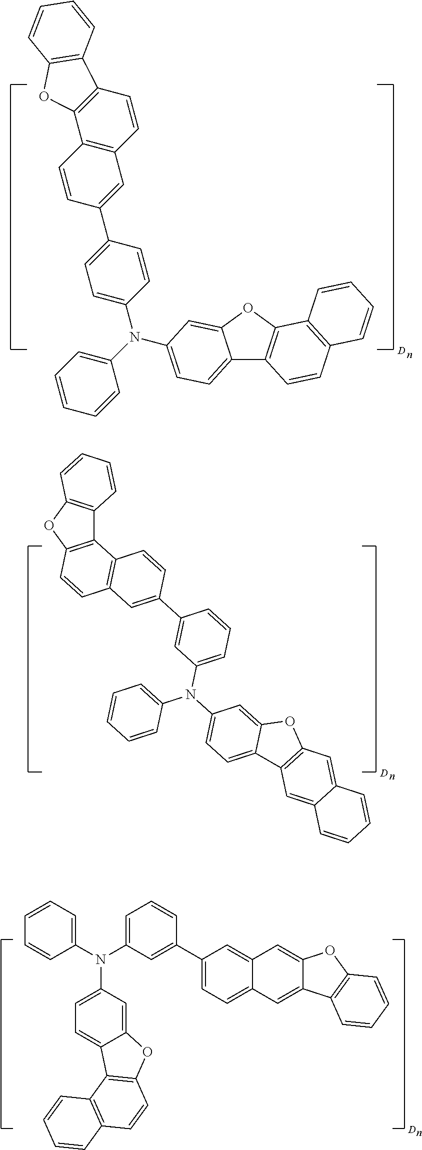

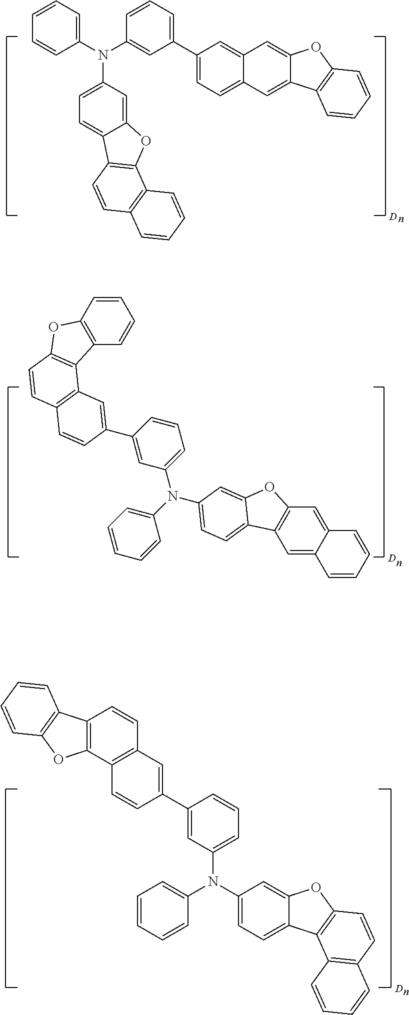

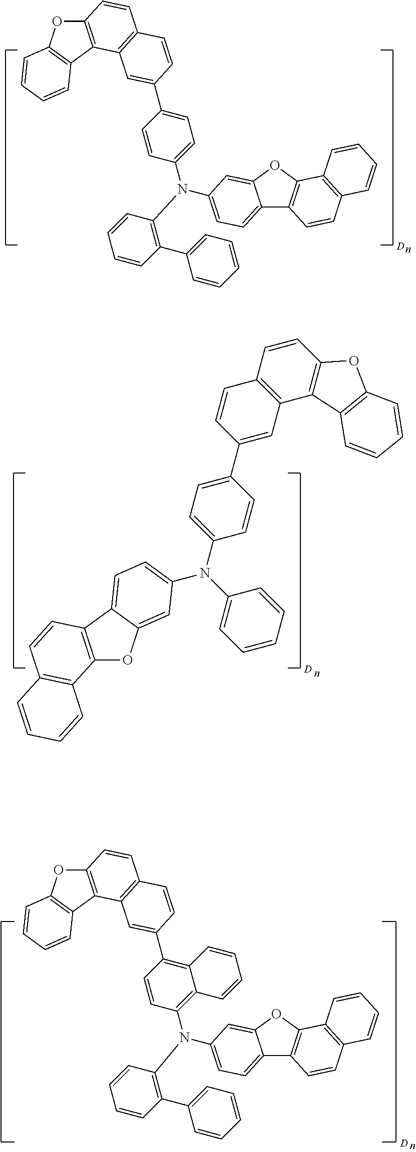

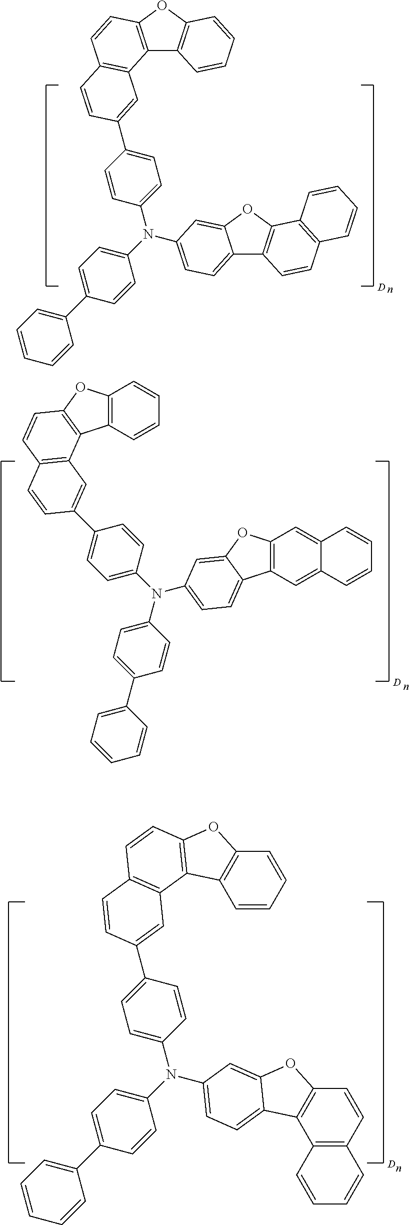

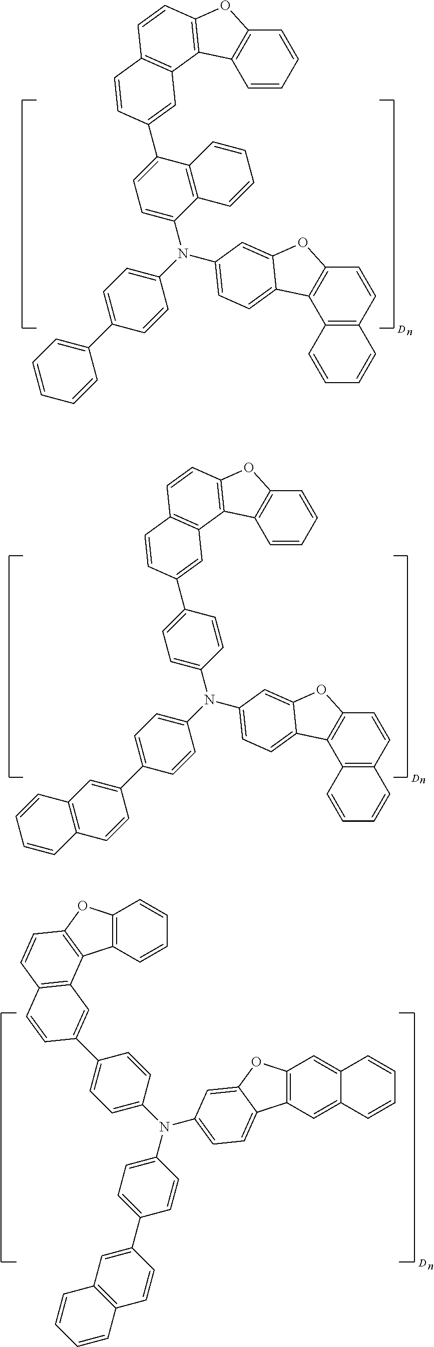

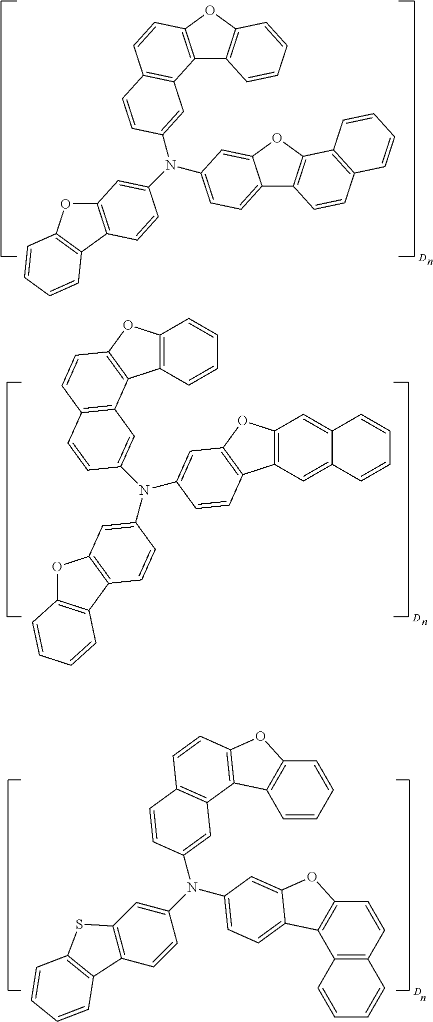

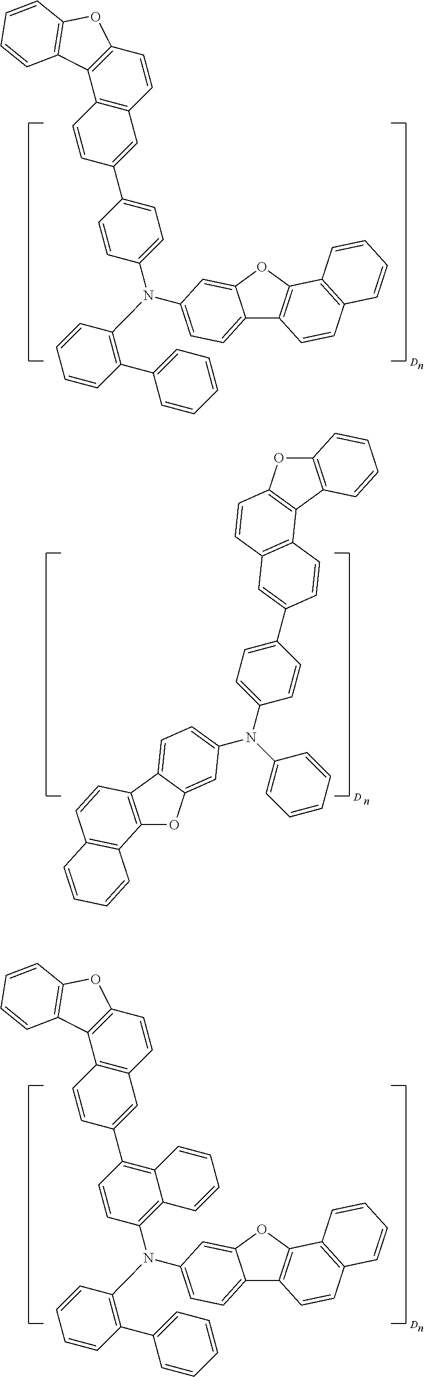

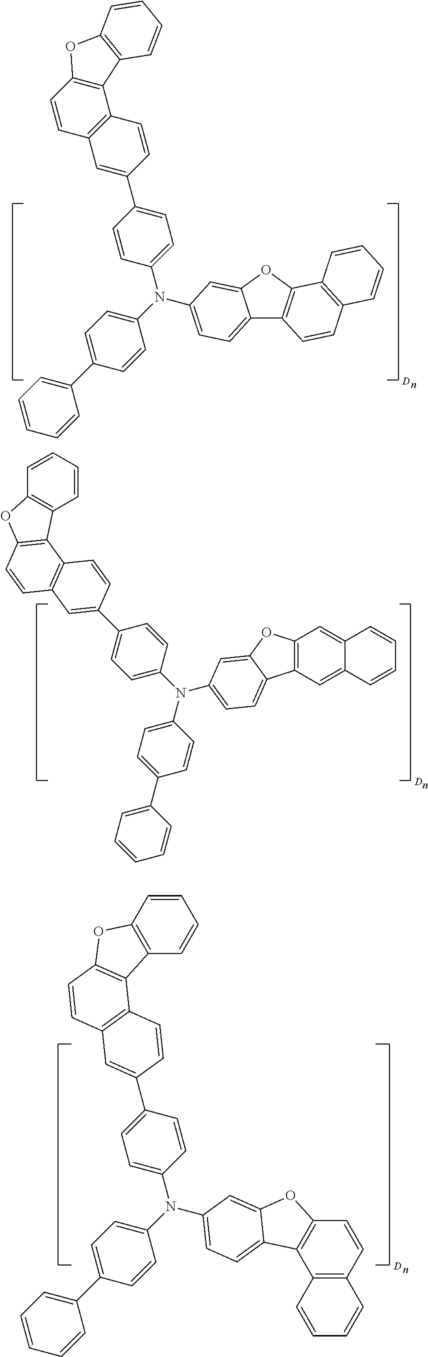

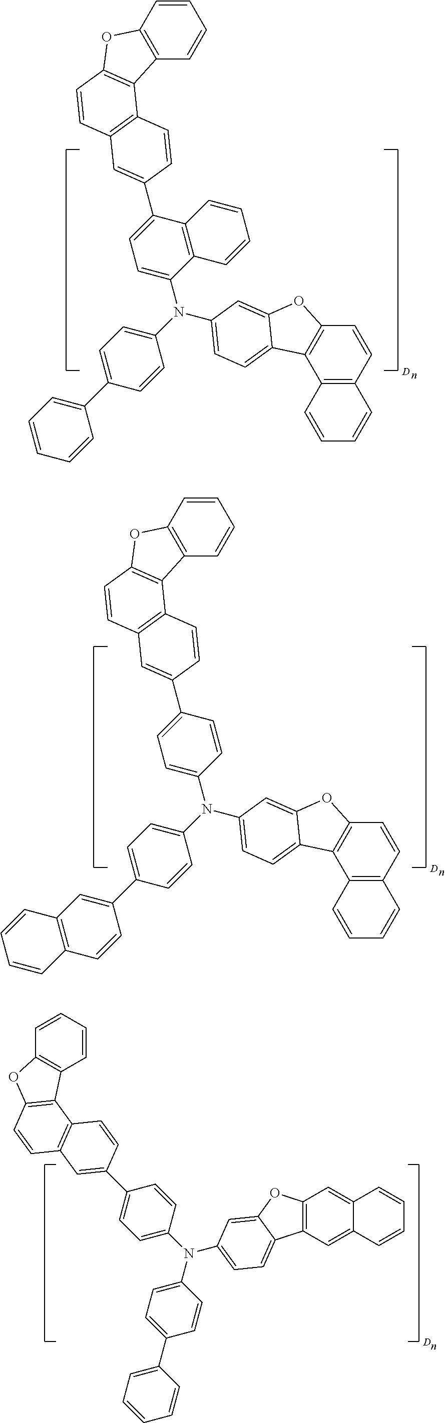

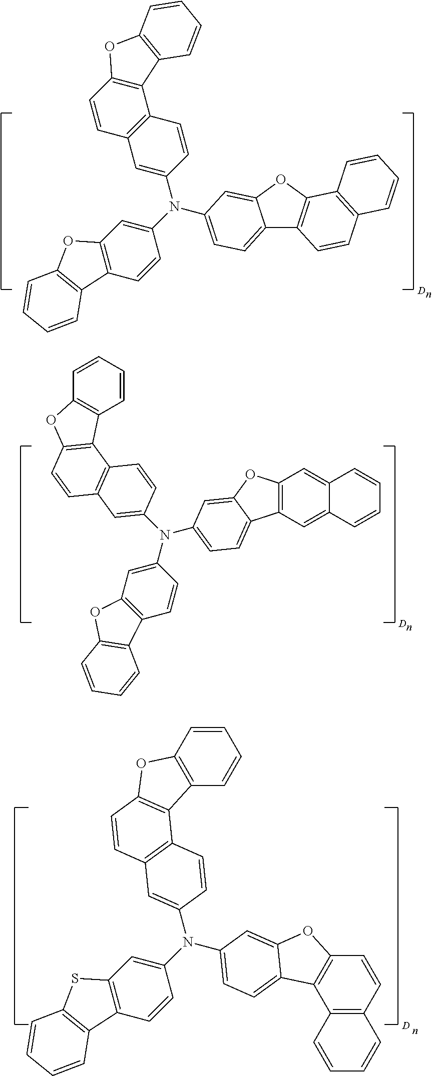

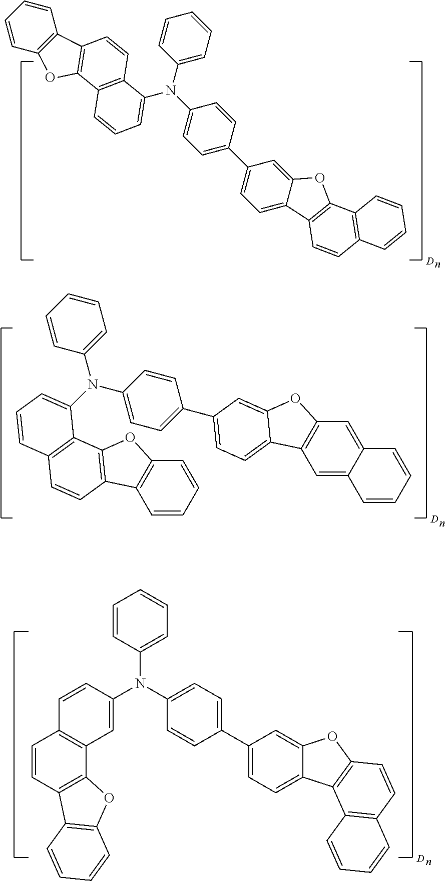

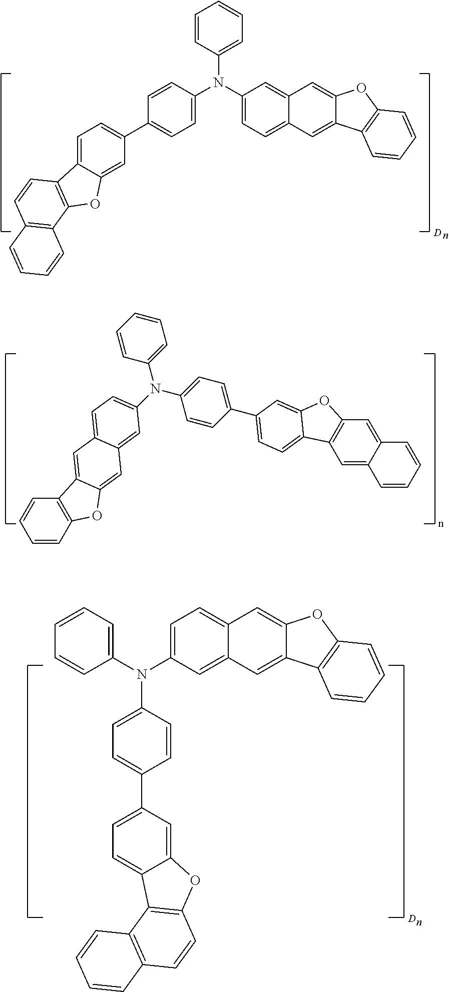

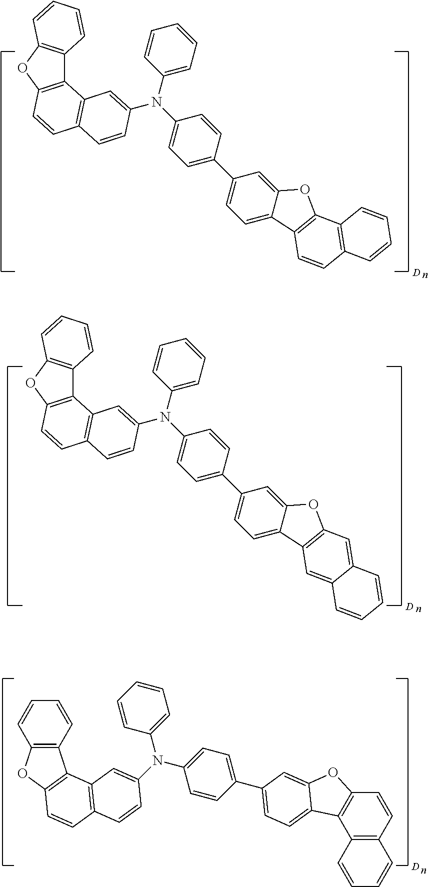

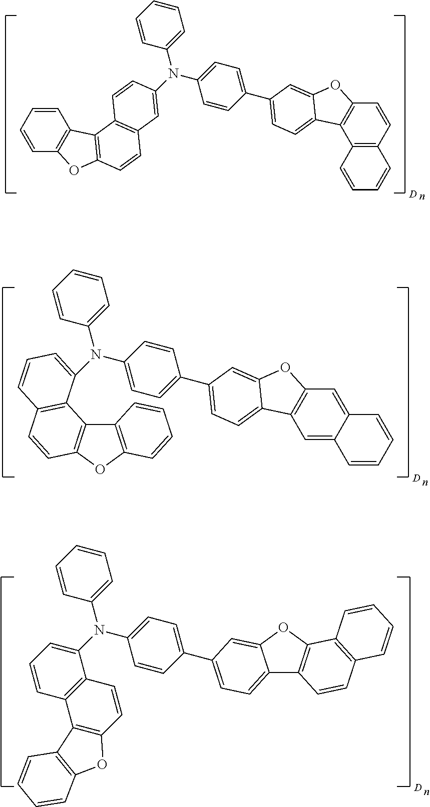

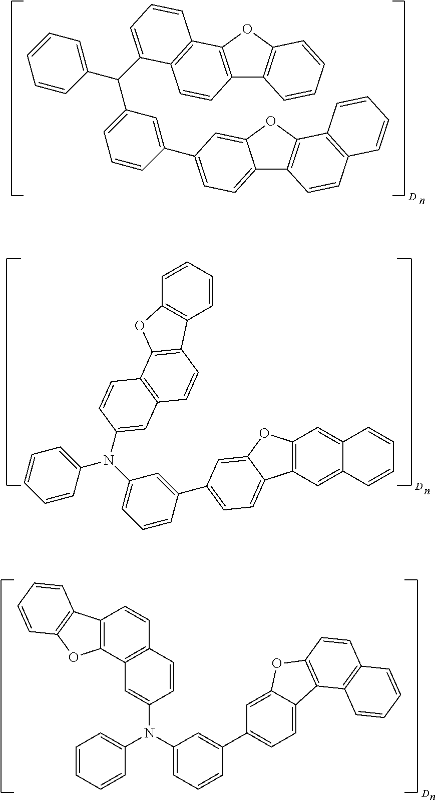

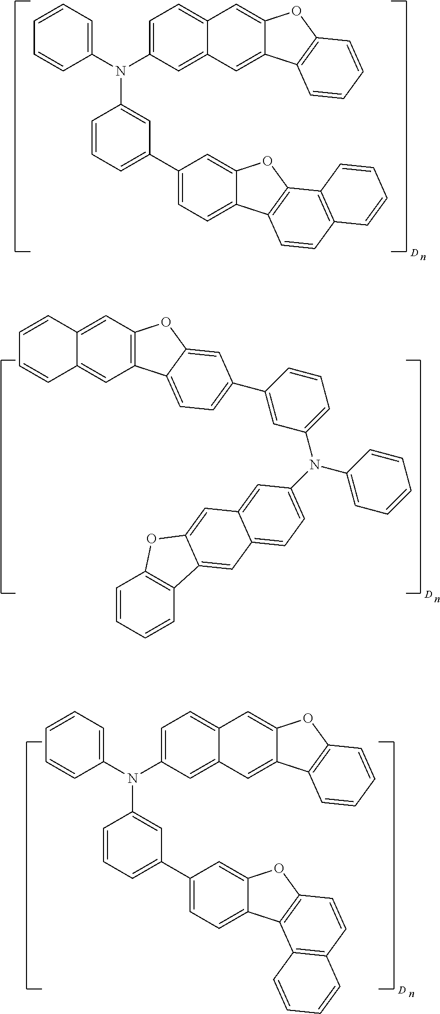

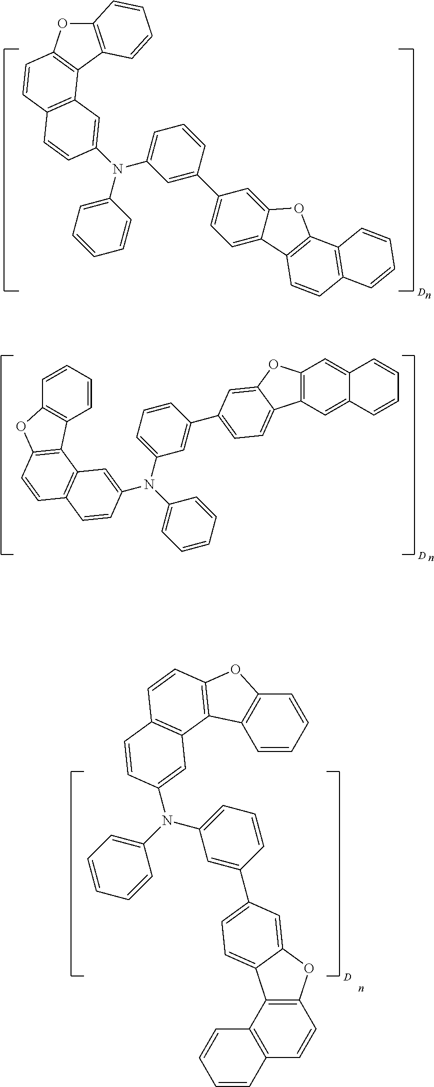

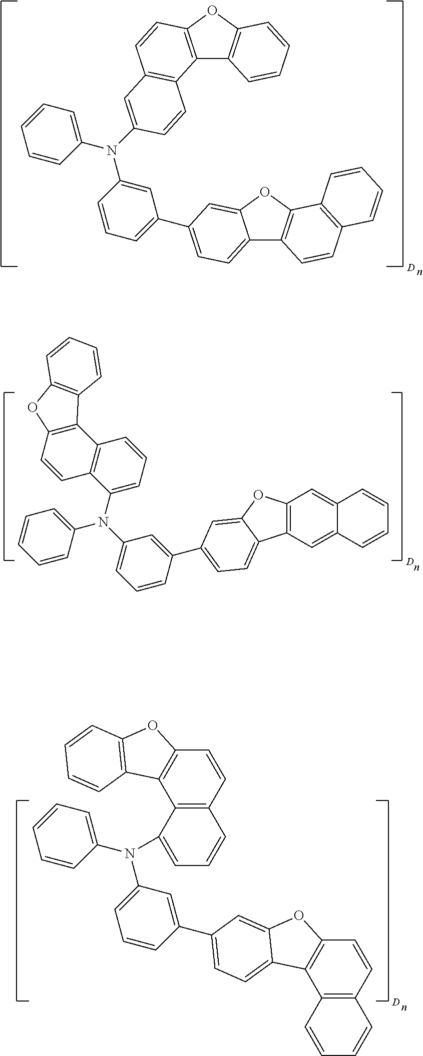

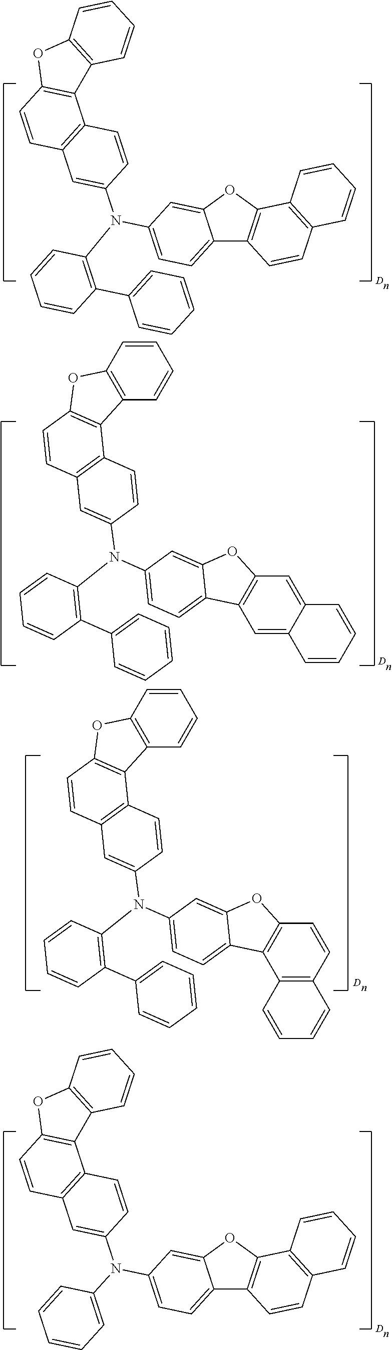

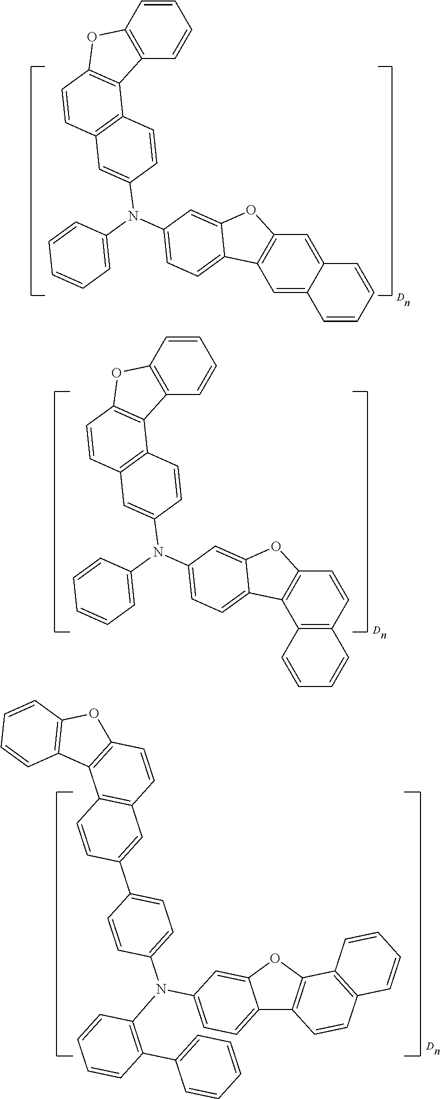

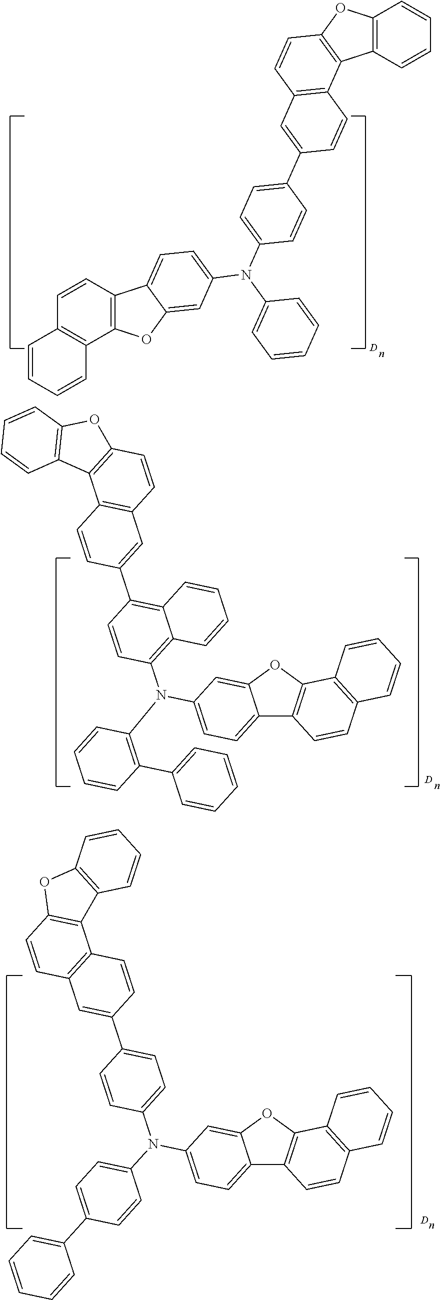

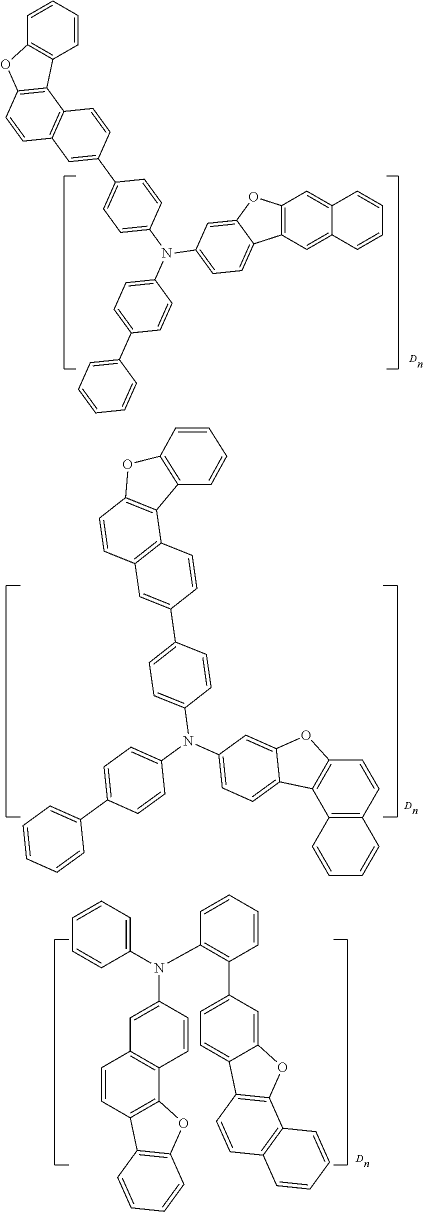

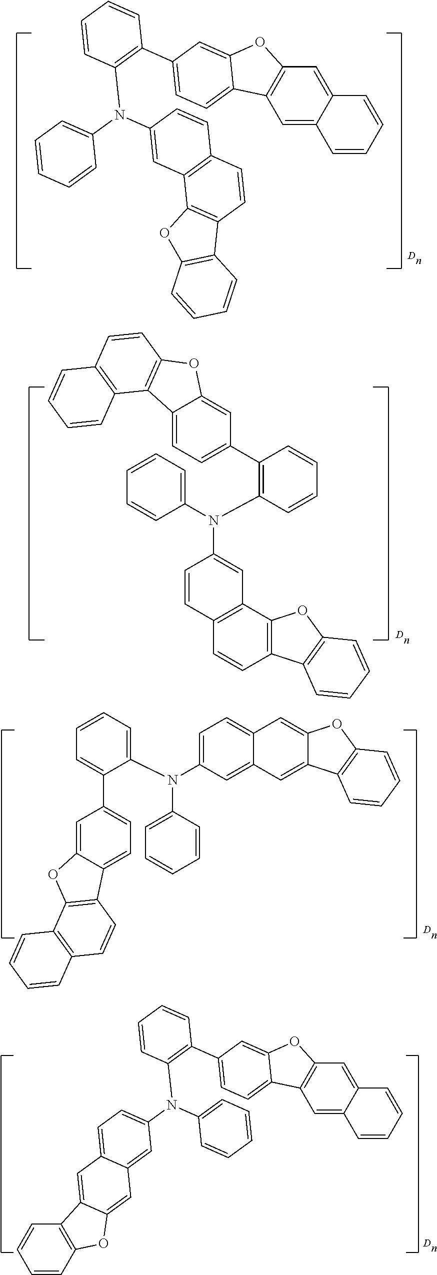

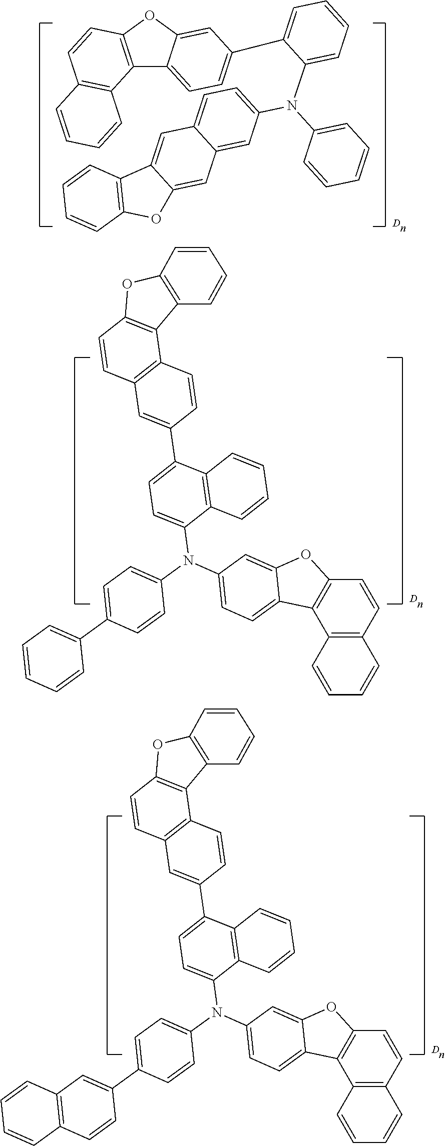

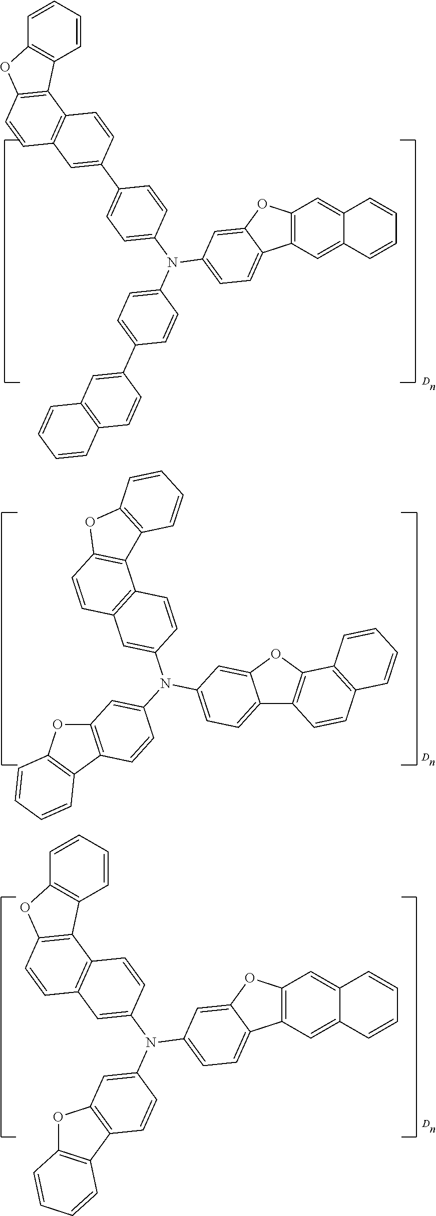

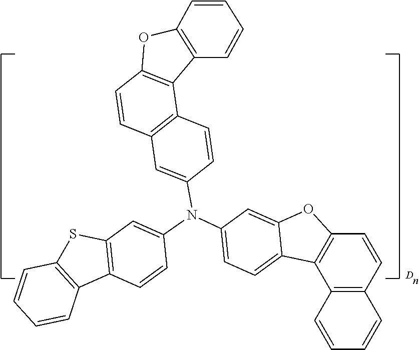

wherein,
Dn means that n hydrogens have been replaced by deuterium,
wherein n is an integer of 1 or more,

Meanwhile, the second compound can be prepared by a preparation method as shown in the following Reaction Scheme 2 as an example:

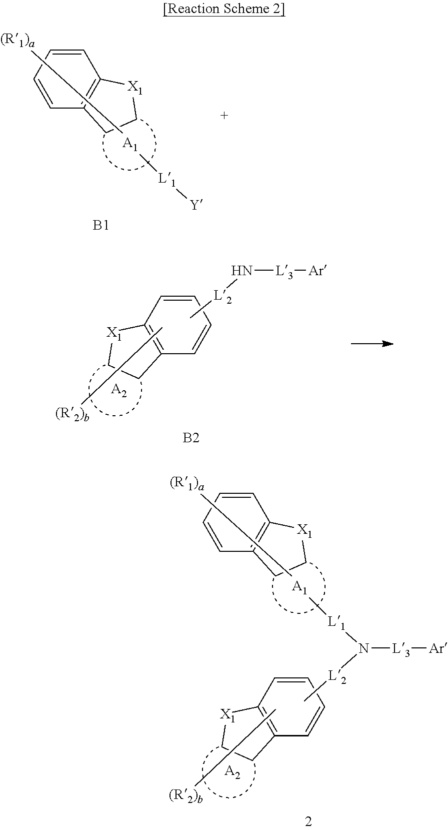

In Reaction Scheme 2, Y′ is halogen, preferably bromo, or chloro, and the definitions of other substituents are the same as described above.

Specifically, the second compound may be prepared by an amine substitution reaction of the starting materials B1 and B2. Such an amine substitution reaction is preferably carried out in the presence of a palladium catalyst and a base, and a reactive group for the amine substitution reaction can be appropriately changed. The preparation method of the second compound can be further embodied in Preparation Examples described hereinafter.

The preparation method of the second compound can be further embodied in Preparation Examples described hereinafter.

Further, the first compound and the second compound may be contained in the light emitting layer at a weight ratio of 1:99 to 99:1. At this time, in the viewpoint of appropriately maintaining the ratio of holes and electrons in the light emitting layer, the first compound and the second compound are preferably contained at a weight ratio of 10:90 to 50:50, or 20:80 to 40:60. Preferably, the first compound and the second compound may be contained in the light emitting layer at a weight ratio of 30:70.

On the other hand, the light emitting layer may further include a dopant material in addition to the two types of host materials. Such a dopant material includes an aromatic amine derivative, a styrylamine compound, a boron complex, a fluoranthene compound, a metal complex, and the like. Specific examples of the aromatic amine derivative include substituted or unsubstituted fused aromatic ring derivatives having a substituted or unsubstituted arylamino group, examples of which include pyrene, anthracene, chrysene, and periflanthene having the arylamino group, and the like. The styrylamine compound is a compound where at least one arylvinyl group is substituted in a substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, examples of the metal complex include an iridium complex, a platinum complex, and the like, but are not limited thereto.

More specifically, the following compounds may be used as the dopant material, but are not limited thereto:

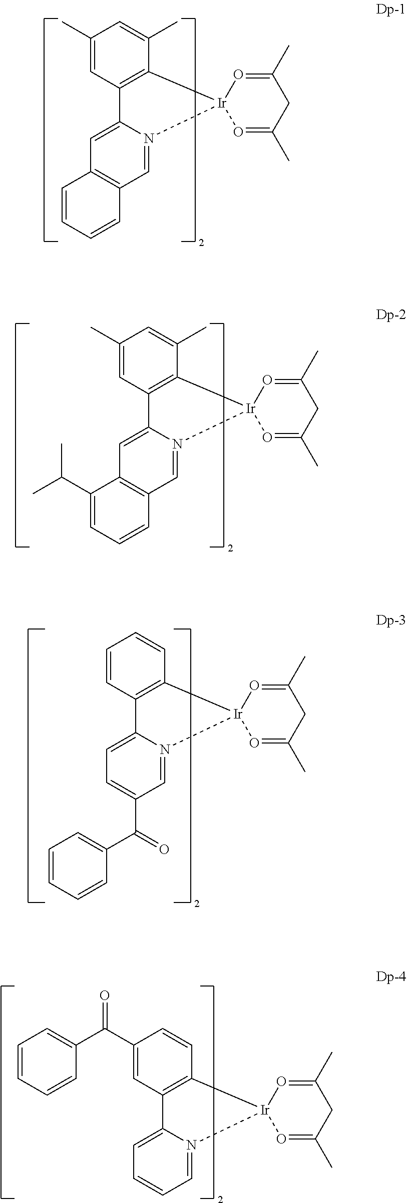

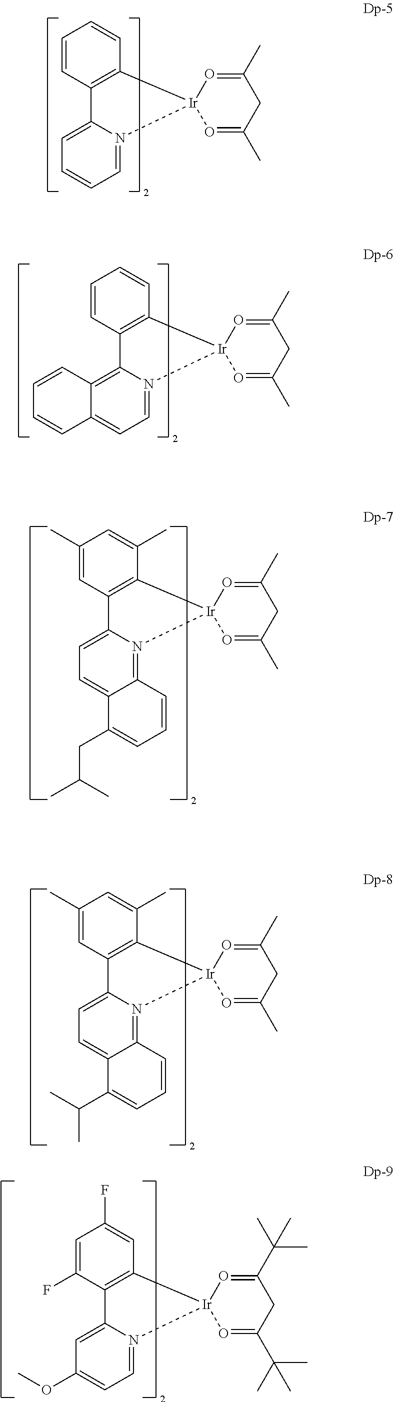

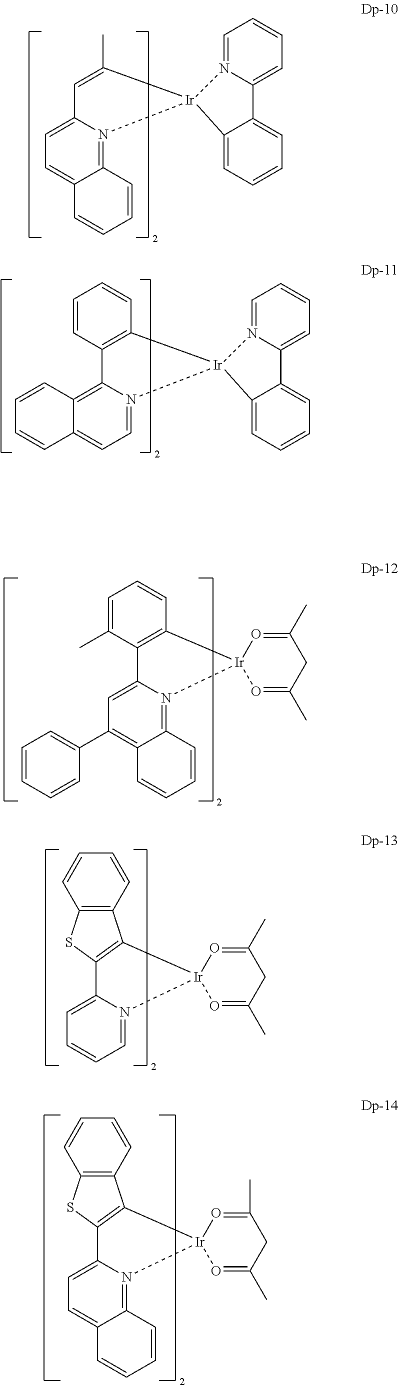

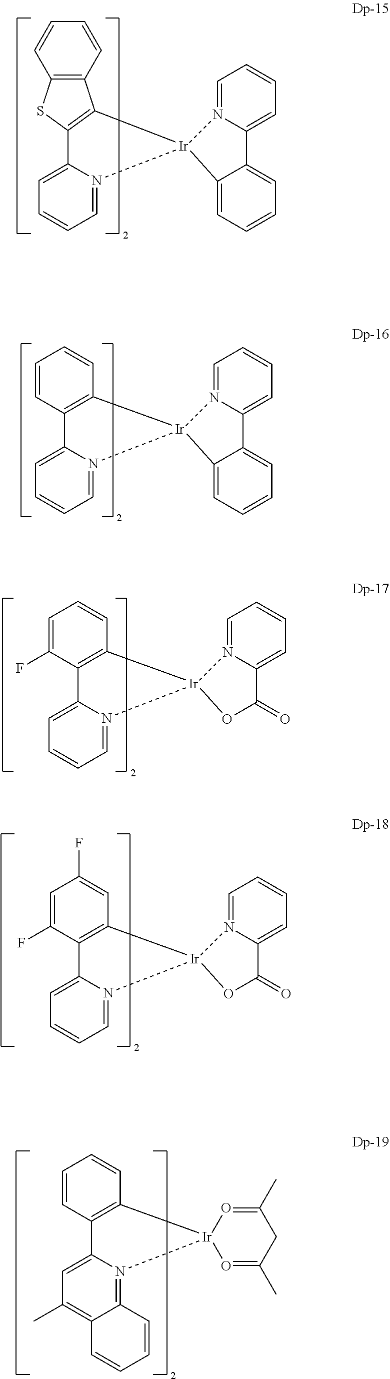

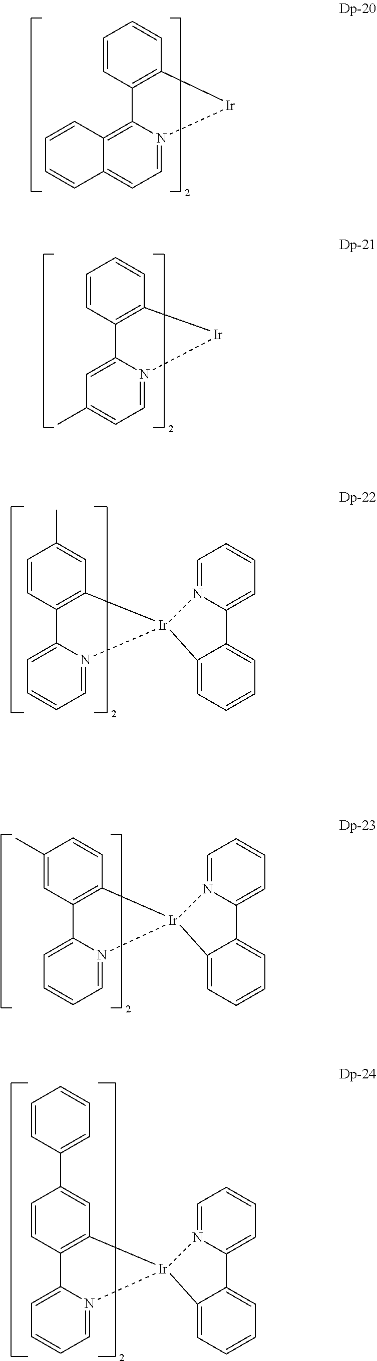

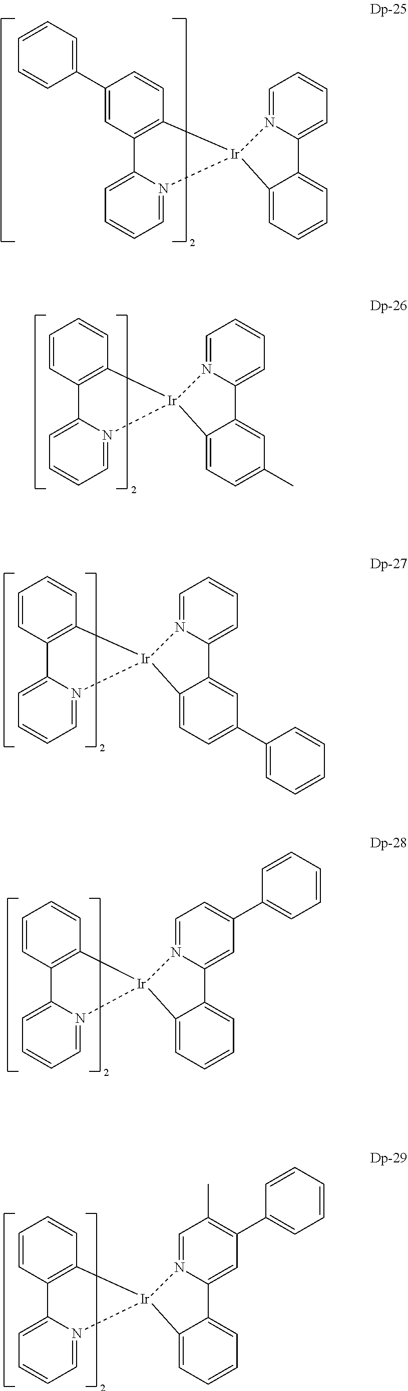

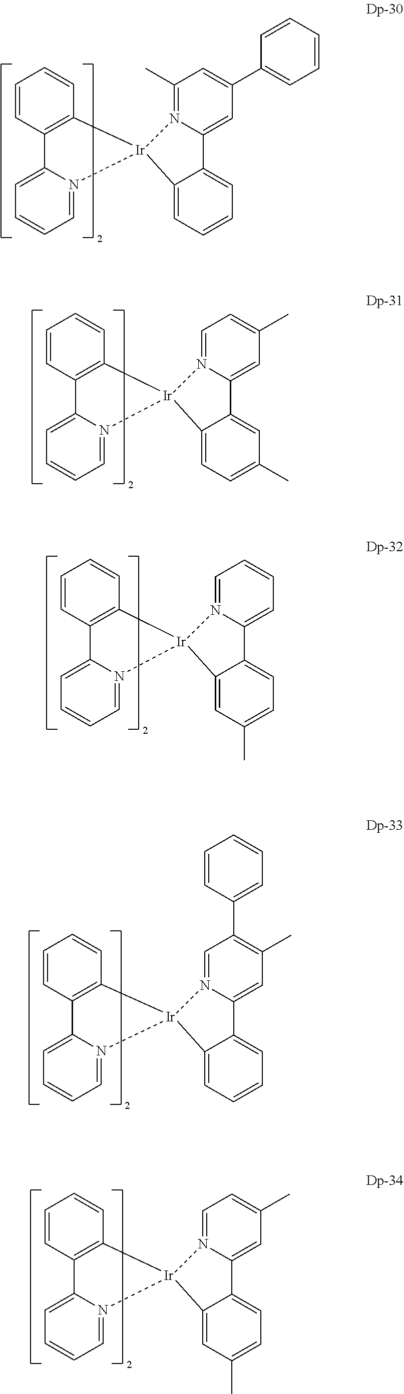

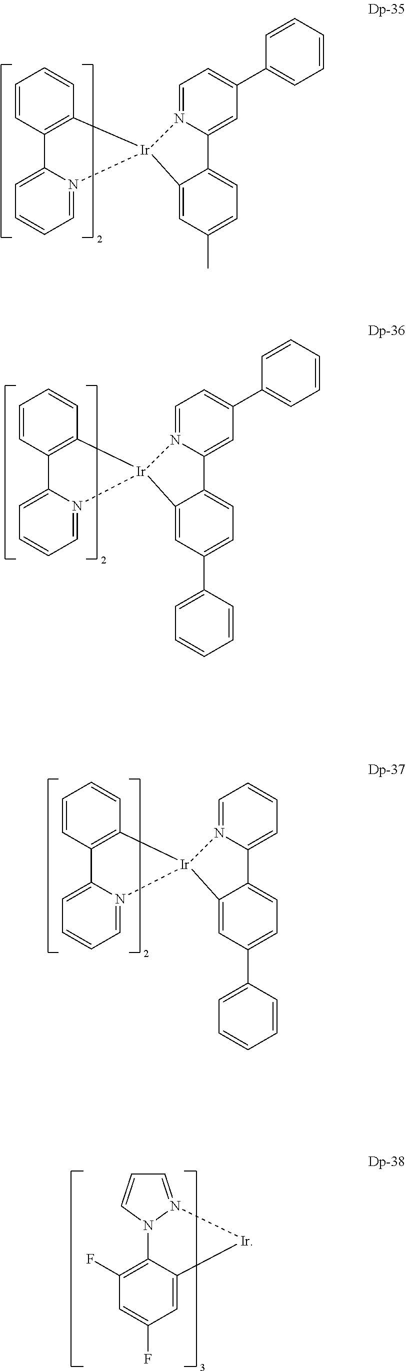

### Hole Blocking Layer

The organic light emitting device according to the present disclosure may include a hole blocking layer between the light emitting layer and an electron transport layer described hereinafter, if necessary. The hole blocking layer refers to a layer which is formed on the light emitting layer, preferably provided in contact with the light emitting layer, and serves to adjust the electron mobility, prevent excessive movement of holes, and increase the probability of hole-electron coupling, thereby improving the efficiency of the organic light emitting device. The hole blocking layer includes a hole blocking material, and examples of such hole blocking material may include a compound having an electron withdrawing group introduced therein, such as azine derivatives including triazine; triazole derivatives; oxadiazole derivatives; phenanthroline derivatives; phosphine oxide derivatives, but is not limited thereto.

### Electron Injection and Transport Layer

The electron injection and transport layer is a layer for simultaneously performing the roles of an electron transport layer and an electron injection layer that inject electrons from an electrode and transport the received electrons up to the light emitting layer, and is formed on the light emitting layer or the hole blocking layer. The electron injection and transport material is suitably a material which can receive electrons well from a cathode and transfer the electrons to a light emitting layer, and has a large mobility for electrons. Specific examples of the electron injection and transport material include: an Al complex of 8-hydroxyquinoline; a complex including Alq₃; an organic radical compound; a hydroxyflavone-metal complex, a triazine derivative, and the like, but are not limited thereto. Alternatively, it may be used together with fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

The electron injection and transport layer may also be formed as a separate layer such as an electron injection layer and an electron transport layer. In such a case, the electron transport layer is formed on the light emitting layer or the hole blocking layer, and the above-mentioned electron injection and transport material may be used as the electron transport material included in the electron transport layer. In addition, the electron injection layer is formed on the electron transport layer, and examples of the electron injection material included in the electron injection layer include LiF, NaCl, CsF, Li₂O, BaO, fluorenone, anthraquinodimethane, diphenoquinone, thiopyran dioxide, oxazole, oxadiazole, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)-beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-hydroxy-quinolinato)(2-naphtholato)gallium, and the like, but are not limited thereto.

### Organic Light Emitting Device

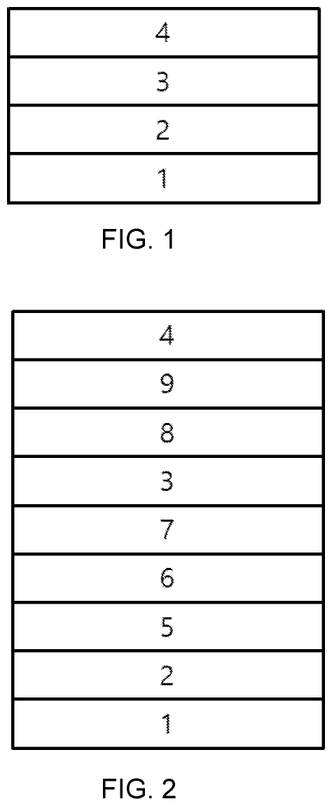

The organic light emitting device according to the present disclosure is illustrated in FIG. 1. FIG. 1 shows an example of an organic light emitting device comprising a substrate 1, an anode 2, a light emitting layer 3, and a cathode 4. In such a structure, the first compound and the second compound can be included in the light emitting layer.

FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron inject and transport layer 9, and a cathode 4.

In such a structure, the first compound and the second compound can be included in the light emitting layer.

The organic light emitting device according to the present disclosure can be manufactured by sequentially stacking the above-described structures. In this case, the organic light emitting device may be manufactured by depositing a metal, metal oxides having conductivity, or an alloy thereof on the substrate by using a PVD (physical vapor deposition) method such as a sputtering method or an e-beam evaporation method to form the anode, forming the respective layers described above thereon, and then depositing a material that can be used as the cathode thereon. In addition to such a method, the organic light emitting device can be manufactured by sequentially depositing from the cathode material to the anode material on a substrate. Further, the light emitting layer may be formed by subjecting hosts and dopants to a vacuum deposition method and a solution coating method. Herein, the solution coating method means a spin coating, a dip coating, a doctor blading, an inkjet printing, a screen printing, a spray method, a roll coating, or the like, but is not limited thereto.

In addition to such a method, the organic light emitting device can be manufactured by sequentially depositing a cathode material, an organic material layer and an anode material on a substrate (International Publication WO2003/012890). However, the manufacturing method is not limited thereto.

The organic light emitting device according to the present disclosure may be a bottom emission type device, a top emission type device, or a double side emission type device, and in particular, it may be a bottom emission type light emitting device that requires relatively high luminous efficiency.

The preparation of the compound represented by Chemical Formula 1, the compound represented by Chemical Formula 2 and the organic light emitting device including the same will be specifically described in the following Examples. However, the following Examples are provided for illustrative purposes only, and are not intended to limit the scope of the present disclosure.

### Synthesis Example 1: Preparation of Compound Represented by Chemical Formula 1

### Synthesis Example 1-1

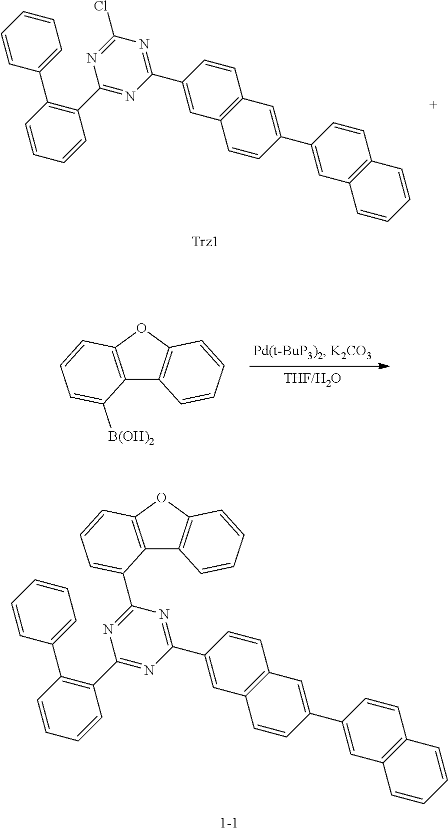 [SMILES: Clc1nc(-c2ccc3cc(-c4ccc5ccccc5c4)ccc3c2)nc(-c2ccccc2-c2ccccc2)n1 ; OB(O)c1cccc2oc3ccccc3c12 ; c1ccc(-c2ccccc2-c2nc(-c3ccc4cc(-c5ccc6ccccc6c5)ccc4c3)nc(-c3cccc4oc5ccccc5c34)n2)cc1]

Trz1 (15 g, 28.8 mmol) and dibenzo[b,d]furan-1-ylboronic acid (6.4 g, 30.3 mmol) were added to 300 mL of THF, and the mixture was stirred and refluxed. Then, potassium carbonate (12 g, 86.5 mmol) was dissolved in 36 ml of water, added thereto, sufficiently stirred, and then bis(tri-tert-butylphosphine)palladium(0) (0.1 g, 0.3 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 12.2 g of Compound 1-1. (Yield: 65%, MS: [M+H]⁺=652).

### Synthesis Example 1-2

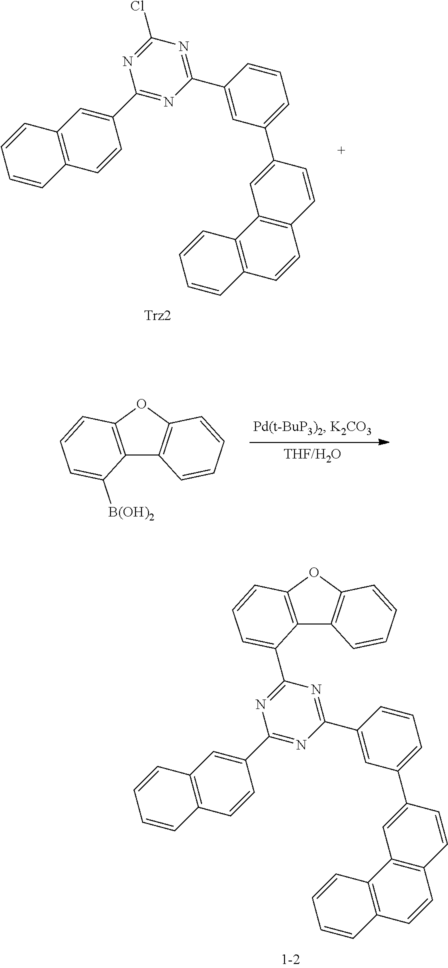 [SMILES: Clc1nc(-c2cccc(-c3ccc4ccc5ccccc5c4c3)c2)nc(-c2ccc3ccccc3c2)n1 ; OB(O)c1cccc2oc3ccccc3c12 ; c1cc(-c2ccc3ccc4ccccc4c3c2)cc(-c2nc(-c3ccc4ccccc4c3)nc(-c3cccc4oc5ccccc5c34)n2)c1]

Trz2 (15 g, 30.4 mmol) and dibenzo[b,d]furan-1-ylboronic acid (6.8 g, 31.9 mmol) were added to 300 mL of THF, and the mixture was stirred and refluxed. Then, potassium carbonate (12.6 g, 91.1 mmol) was dissolved in 38 ml of water, added thereto, sufficiently stirred, and then bis(tri-tert-butylphosphine)palladium(0) (0.2 g, 0.3 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 14 g of Compound 1-2. (Yield: 74%, MS: [M+H]⁺=626).

### Synthesis Example 1-3

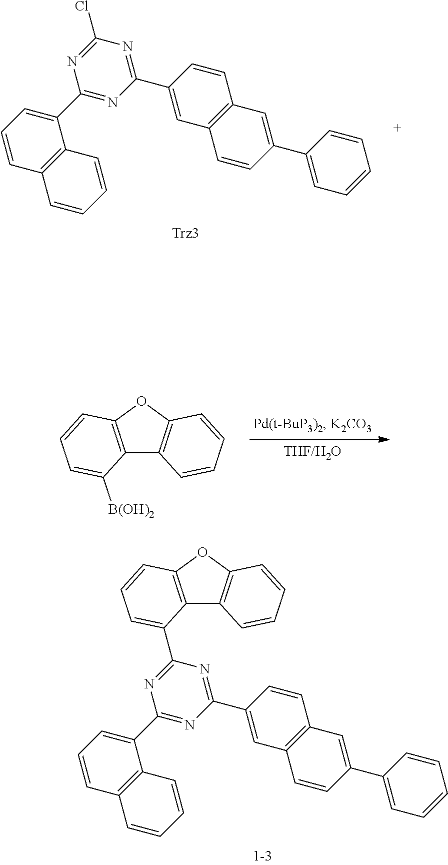 [SMILES: Clc1nc(-c2ccc3cc(-c4ccccc4)ccc3c2)nc(-c2cccc3ccccc23)n1 ; OB(O)c1cccc2oc3ccccc3c12 ; c1ccc(-c2ccc3cc(-c4nc(-c5cccc6ccccc56)nc(-c5cccc6oc7ccccc7c56)n4)ccc3c2)cc1]

Trz3 (15 g, 33.8 mmol) and dibenzo[b,d]furan-1-ylboronic acid (7.5 g, 35.5 mmol) were added to 300 mL of THF, and the mixture was stirred and refluxed. Then, potassium carbonate (14 g, 101.4 mmol) was dissolved in 42 ml of water, added thereto, sufficiently stirred, and then bis(tri-tert-butylphosphine)palladium(0) (0.2 g, 0.3 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.4 g of Compound 1-3. (Yield: 69%, MS: [M+H]⁺=576).

### Synthesis Example 1-4

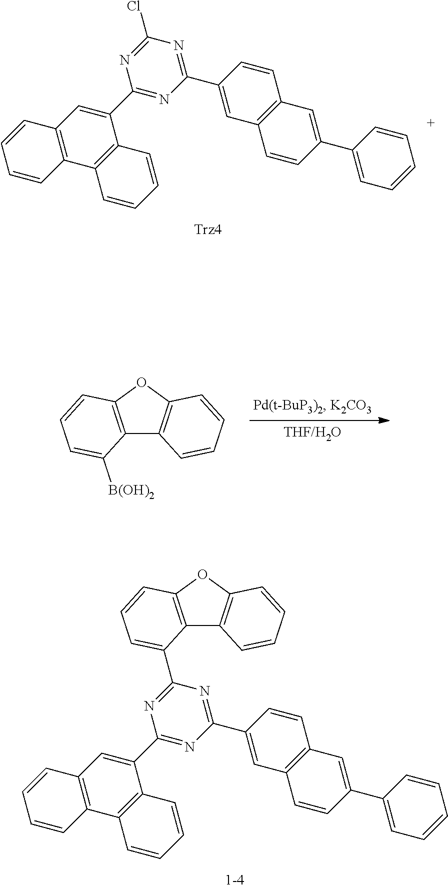 [SMILES: Clc1nc(-c2ccc3cc(-c4ccccc4)ccc3c2)nc(-c2cc3ccccc3c3ccccc23)n1 ; OB(O)c1cccc2oc3ccccc3c12 ; c1ccc(-c2ccc3cc(-c4nc(-c5cc6ccccc6c6ccccc56)nc(-c5cccc6oc7ccccc7c56)n4)ccc3c2)cc1]

Trz4 (15 g, 30.4 mmol) and dibenzo[b,d]furan-1-ylboronic acid (6.8 g, 31.9 mmol) were added to 300 mL of THF, and the mixture was stirred and refluxed. Then, potassium carbonate (12.6 g, 91.1 mmol) was dissolved in 38 ml of water, added thereto, sufficiently stirred, and then bis(tri-tert-butylphosphine)palladium(0) (0.2 g, 0.3 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.3 g of Compound 1-4. (Yield: 70%, MS: [M+H]⁺=626).

### Synthesis Example 1-5

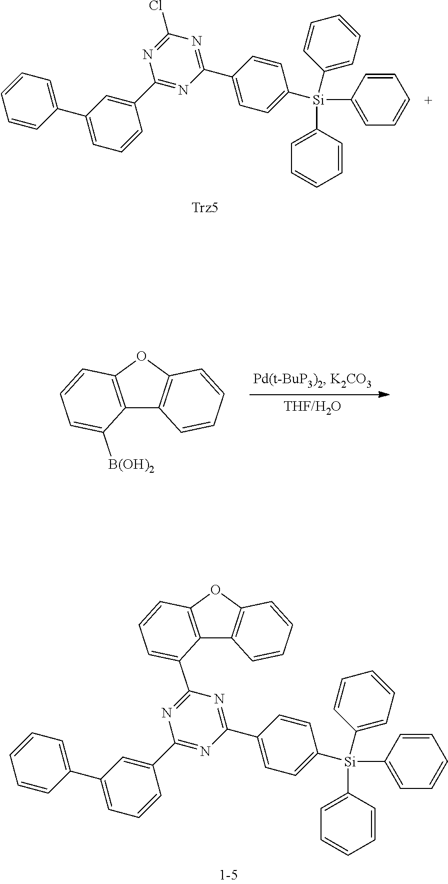 [SMILES: Clc1nc(-c2ccc([Si](c3ccccc3)(c3ccccc3)c3ccccc3)cc2)nc(-c2cccc(-c3ccccc3)c2)n1 ; OB(O)c1cccc2oc3ccccc3c12 ; c1ccc(-c2cccc(-c3nc(-c4ccc([Si](c5ccccc5)(c5ccccc5)c5ccccc5)cc4)nc(-c4cccc5oc6ccccc6c45)n3)c2)cc1]

Trz5 (15 g, 24.9 mmol) and dibenzo[b,d]furan-1-ylboronic acid (5.5 g, 26.2 mmol) were added to 300 mL of THF, and the mixture was stirred and refluxed. Then, potassium carbonate (10.3 g, 74.7 mmol) was dissolved in 31 ml of water, added thereto, sufficiently stirred, and then bis(tri-tert-butylphosphine)palladium(0) (0.1 g, 0.2 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 12.6 g of Compound 1-5. (Yield: 69%, MS: [M+H]⁺=734).

### Synthesis Example 1-6

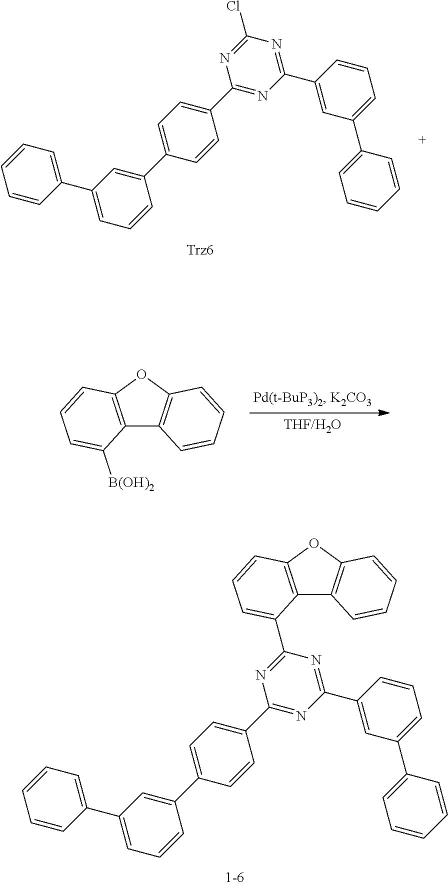 [SMILES: Clc1nc(-c2ccc(-c3cccc(-c4ccccc4)c3)cc2)nc(-c2cccc(-c3ccccc3)c2)n1 ; OB(O)c1cccc2oc3ccccc3c12 ; c1ccc(-c2cccc(-c3ccc(-c4nc(-c5cccc(-c6ccccc6)c5)nc(-c5cccc6oc7ccccc7c56)n4)cc3)c2)cc1]

Trz6 (15 g, 30.2 mmol) and dibenzo[b,d]furan-1-ylboronic acid (6.7 g, 31.8 mmol) were added to 300 mL of THF, and the mixture was stirred and refluxed. Then, potassium carbonate (12.5 g, 90.7 mmol) was dissolved in 38 ml of water, added thereto, sufficiently stirred, and then bis(tri-tert-butylphosphine)palladium(0) (0.2 g, 0.3 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 12.5 g of Compound 1-6. (Yield: 66%, MS: [M+H]⁺=629).

### Synthesis Example 1-7

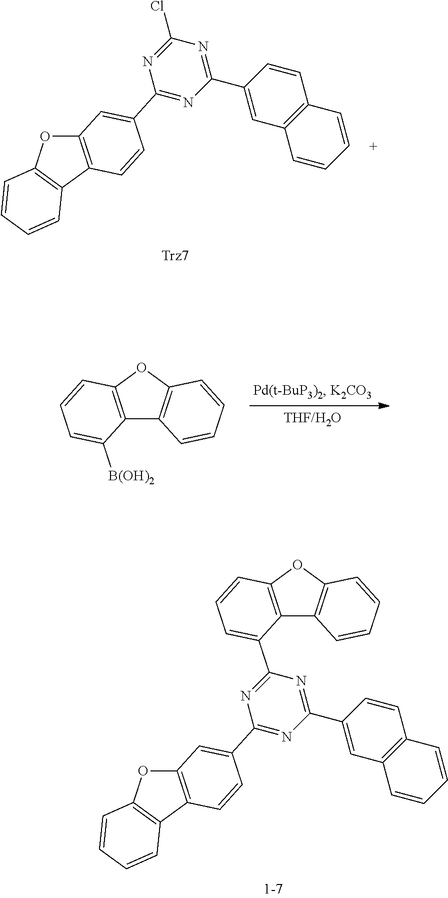 [SMILES: Clc1nc(-c2ccc3ccccc3c2)nc(-c2ccc3c(c2)oc2ccccc23)n1 ; OB(O)c1cccc2oc3ccccc3c12 ; c1ccc2cc(-c3nc(-c4ccc5c(c4)oc4ccccc45)nc(-c4cccc5oc6ccccc6c45)n3)ccc2c1]

Trz7 (15 g, 36.8 mmol) and dibenzo[b,d]furan-1-ylboronic acid (8.2 g, 38.6 mmol) were added to 300 mL of THF, and the mixture was stirred and refluxed. Then, potassium carbonate (15.2 g, 110.3 mmol) was dissolved in 46 ml of water, added thereto, sufficiently stirred, and then bis(tri-tert-butylphosphine)palladium(0) (0.2 g, 0.4 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 14.9 g of Compound 1-7. (Yield: 75%, MS: [M+H]⁺=540).

### Synthesis Example 1-8

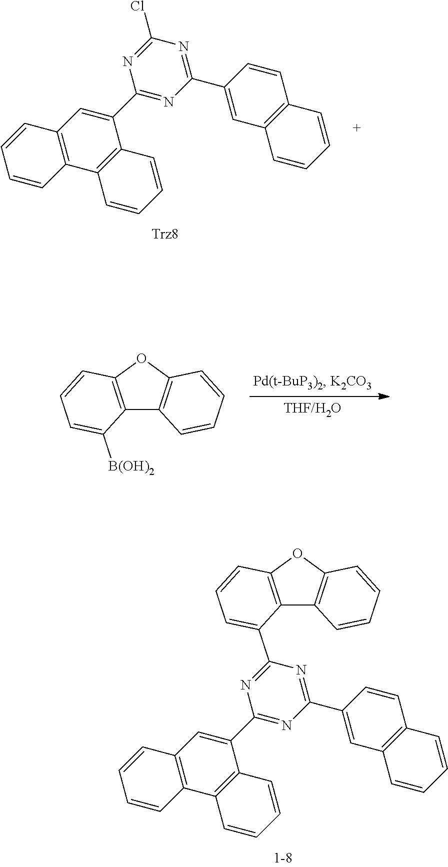 [SMILES: Clc1nc(-c2ccc3ccccc3c2)nc(-c2cc3ccccc3c3ccccc23)n1 ; OB(O)c1cccc2oc3ccccc3c12 ; c1ccc2cc(-c3nc(-c4cc5ccccc5c5ccccc45)nc(-c4cccc5oc6ccccc6c45)n3)ccc2c1]

Trz8 (15 g, 35.9 mmol) and dibenzo[b,d]furan-1-ylboronic acid (8 g, 37.7 mmol) were added to 300 mL of THF, and the mixture was stirred and refluxed. Then, potassium carbonate (14.9 g, 107.7 mmol) was dissolved in 45 ml of water, added thereto, sufficiently stirred, and then bis(tri-tert-butylphosphine)palladium(0) (0.2 g, 0.4 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.8 g of Compound 1-8. (Yield: 70%, MS: [M+H]⁺=550).

### Synthesis Example 1-9

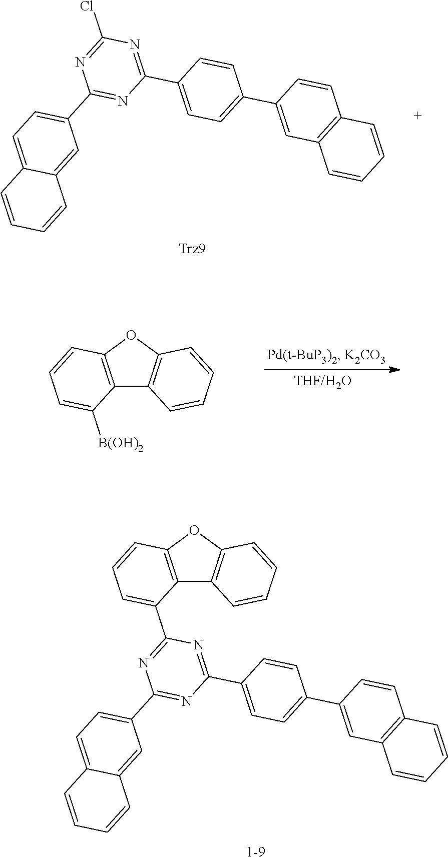 [SMILES: Clc1nc(-c2ccc(-c3ccc4ccccc4c3)cc2)nc(-c2ccc3ccccc3c2)n1 ; OB(O)c1cccc2oc3ccccc3c12 ; c1ccc2cc(-c3ccc(-c4nc(-c5ccc6ccccc6c5)nc(-c5cccc6oc7ccccc7c56)n4)cc3)ccc2c1]

Trz9 (15 g, 33.8 mmol) and dibenzo[b,d]furan-1-ylboronic acid (7.5 g, 35.5 mmol) were added to 300 mL of THF, and the mixture was stirred and refluxed. Then, potassium carbonate (14 g, 101.4 mmol) was dissolved in 42 ml of water, added thereto, sufficiently stirred, and then bis(tri-tert-butylphosphine)palladium(0) (0.2 g, 0.3 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.6 g of Compound 1-9. (Yield: 70%, MS: [M+H]⁺=576).

### Synthesis Example 1-10

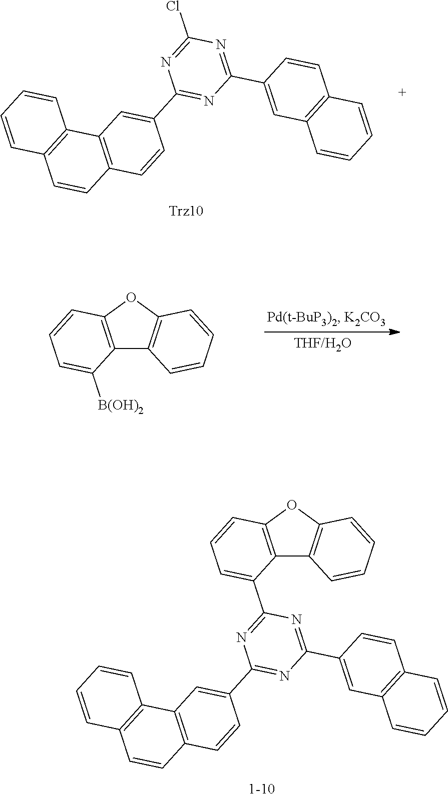 [SMILES: Clc1nc(-c2ccc3ccccc3c2)nc(-c2ccc3ccc4ccccc4c3c2)n1 ; OB(O)c1cccc2oc3ccccc3c12 ; c1ccc2cc(-c3nc(-c4ccc5ccc6ccccc6c5c4)nc(-c4cccc5oc6ccccc6c45)n3)ccc2c1]

Trz10 (15 g, 35.9 mmol) and dibenzo[b,d]furan-1-ylboronic acid (8 g, 37.7 mmol) were added to 300 mL of THF, and the mixture was stirred and refluxed. Then, potassium carbonate (14.9 g, 107.7 mmol) was dissolved in 45 ml of water, added thereto, sufficiently stirred, and then bis(tri-tert-butylphosphine)palladium(0) (0.2 g, 0.4 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.8 g of Compound 1-10. (Yield: 70%, MS: [M+H]⁺=550).

### Synthesis Example 1-11

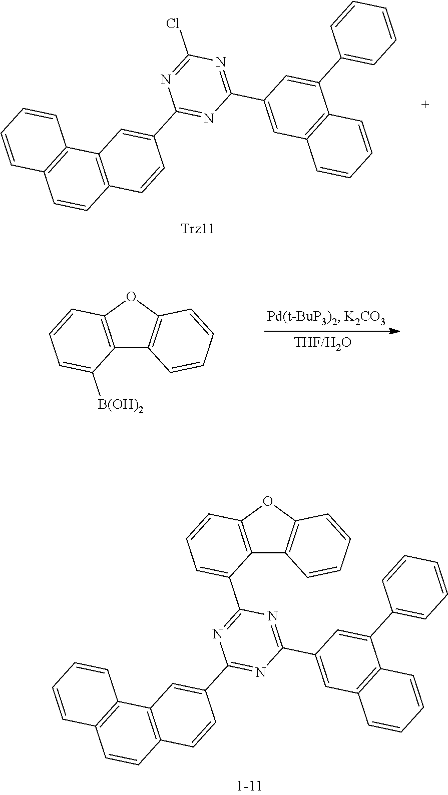 [SMILES: Clc1nc(-c2cc(-c3ccccc3)c3ccccc3c2)nc(-c2ccc3ccc4ccccc4c3c2)n1 ; OB(O)c1cccc2oc3ccccc3c12 ; c1ccc(-c2cc(-c3nc(-c4ccc5ccc6ccccc6c5c4)nc(-c4cccc5oc6ccccc6c45)n3)cc3ccccc23)cc1]

Trz11 (15 g, 30.4 mmol) and dibenzo[b,d]furan-1-ylboronic acid (6.8 g, 31.9 mmol) were added to 300 mL of THF, and the mixture was stirred and refluxed. Then, potassium carbonate (12.6 g, 91.1 mmol) was dissolved in 38 ml of water, added thereto, sufficiently stirred, and then bis(tri-tert-butylphosphine)palladium(0) (0.2 g, 0.3 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.7 g of Compound 1-11. (Yield: 72%, MS: [M+H]⁺=626).

### Synthesis Example 1-12

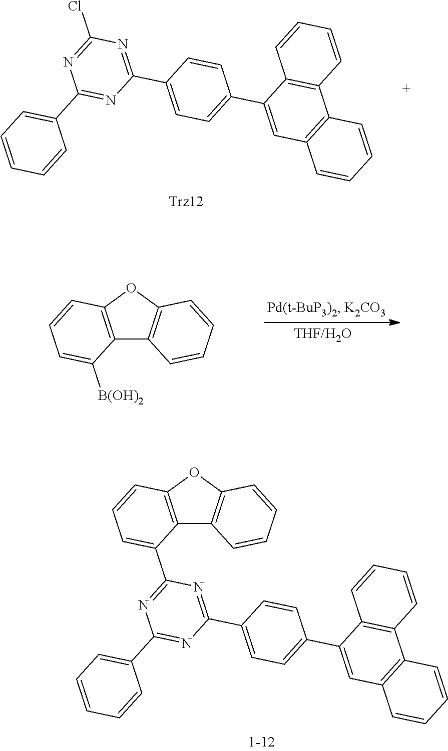 [SMILES: Clc1nc(-c2ccccc2)nc(-c2ccc(-c3cc4ccccc4c4ccccc34)cc2)n1 ; OB(O)c1cccc2oc3ccccc3c12 ; c1ccc(-c2nc(-c3ccc(-c4cc5ccccc5c5ccccc45)cc3)nc(-c3cccc4oc5ccccc5c34)n2)cc1]

Trz12 (15 g, 33.8 mmol) and dibenzo[b,d]furan-1-ylboronic acid (7.5 g, 35.5 mmol) were added to 300 mL of THF, and the mixture was stirred and refluxed. Then, potassium carbonate (14 g, 101.4 mmol) was dissolved in 42 ml of water, added thereto, sufficiently stirred, and then bis(tri-tert-butylphosphine)palladium(0) (0.2 g, 0.3 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 14.2 g of Compound 1-12. (Yield: 73%, MS: [M+H]⁺=576).

### Synthesis Example 1-13

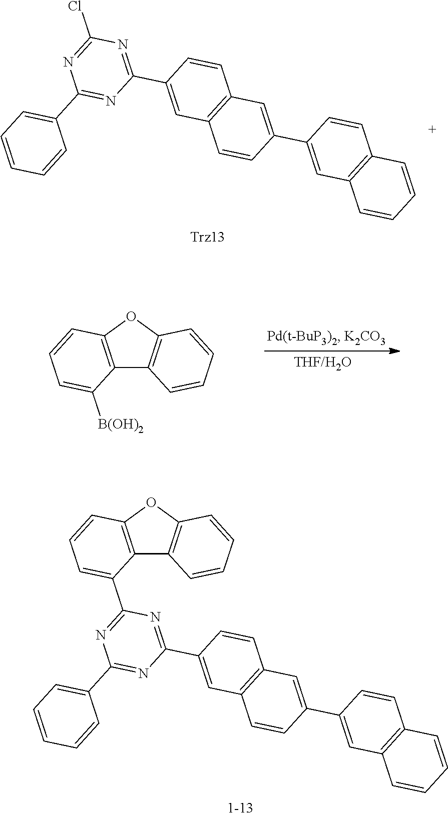 [SMILES: Clc1nc(-c2ccccc2)nc(-c2ccc3cc(-c4ccc5ccccc5c4)ccc3c2)n1 ; OB(O)c1cccc2oc3ccccc3c12 ; c1ccc(-c2nc(-c3ccc4cc(-c5ccc6ccccc6c5)ccc4c3)nc(-c3cccc4oc5ccccc5c34)n2)cc1]

Trz13 (15 g, 33.8 mmol) and dibenzo[b,d]furan-1-ylboronic acid (7.5 g, 35.5 mmol) were added to 300 mL of THF, and the mixture was stirred and refluxed. Then, potassium carbonate (14 g, 101.4 mmol) was dissolved in 42 ml of water, added thereto, sufficiently stirred, and then bis(tri-tert-butylphosphine)palladium(0) (0.2 g, 0.3 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.4 g of Compound 1-13. (Yield: 69%, MS: [M+H]⁺=576).

### Synthesis Example 1-14

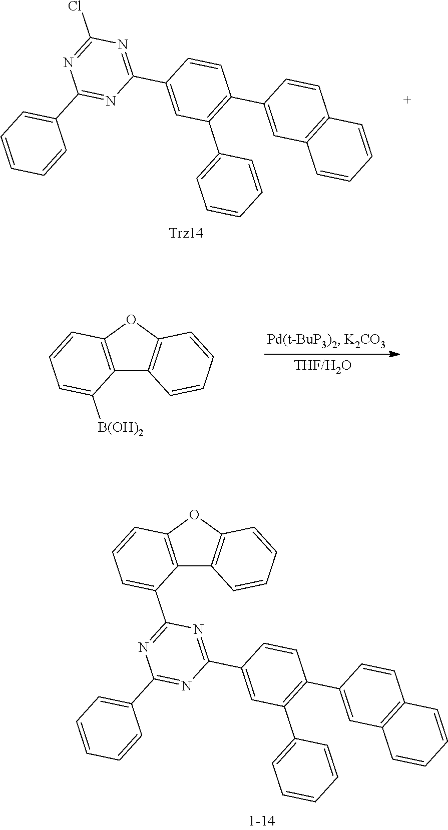 [SMILES: Clc1nc(-c2ccccc2)nc(-c2ccc(-c3ccc4ccccc4c3)c(-c3ccccc3)c2)n1 ; OB(O)c1cccc2oc3ccccc3c12 ; c1ccc(-c2nc(-c3ccc(-c4ccc5ccccc5c4)c(-c4ccccc4)c3)nc(-c3cccc4oc5ccccc5c34)n2)cc1]

Trz14 (15 g, 31.9 mmol) and dibenzo[b,d]furan-1-ylboronic acid (7.1 g, 33.5 mmol) were added to 300 mL of THF, and the mixture was stirred and refluxed. Then, potassium carbonate (13.2 g, 95.8 mmol) was dissolved in 40 ml of water, added thereto, sufficiently stirred, and then bis(tri-tert-butylphosphine)palladium(0) (0.2 g, 0.3 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 14.2 g of Compound 1-14. (Yield: 74%, MS: [M+H]⁺=602).

### Synthesis Example 1-15

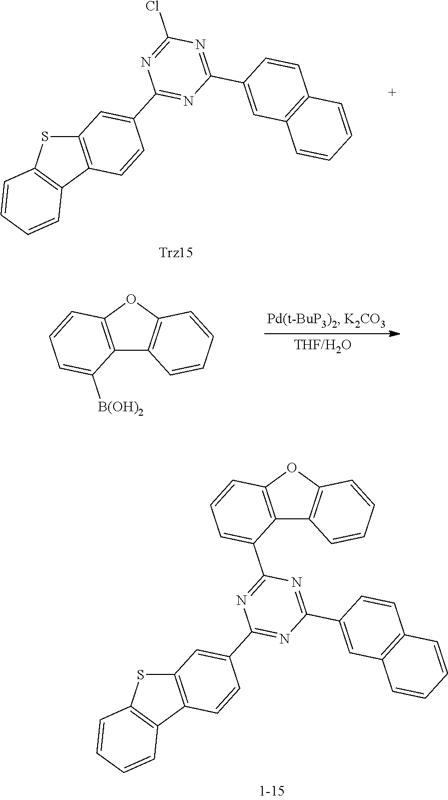 [SMILES: Clc1nc(-c2ccc3ccccc3c2)nc(-c2ccc3c(c2)sc2ccccc23)n1 ; OB(O)c1cccc2oc3ccccc3c12 ; c1ccc2cc(-c3nc(-c4ccc5c(c4)sc4ccccc45)nc(-c4cccc5oc6ccccc6c45)n3)ccc2c1]

Trz15 (15 g, 35.4 mmol) and dibenzo[b,d]furan-1-ylboronic acid (7.9 g, 37.2 mmol) were added to 300 mL of THF, and the mixture was stirred and refluxed. Then, potassium carbonate (14.7 g, 106.2 mmol) was dissolved in 44 ml of water, added thereto, sufficiently stirred, and then bis(tri-tert-butylphosphine)palladium(0) (0.2 g, 0.4 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 14.3 g of Compound 1-15. (Yield: 73%, MS: [M+H]⁺=556).

### Synthesis Example 1-16

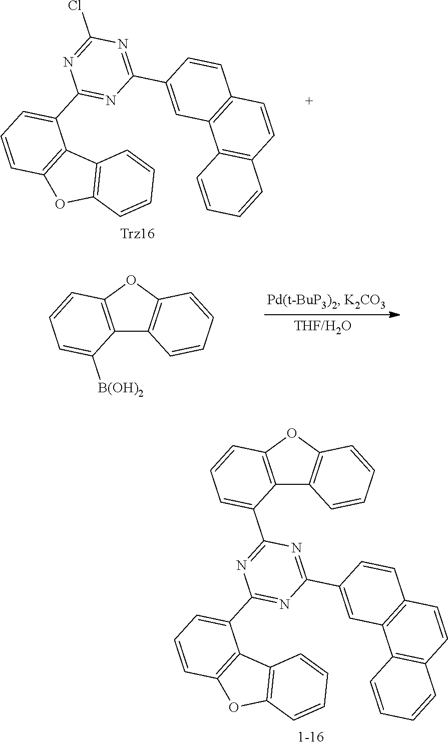 [SMILES: Clc1nc(-c2ccc3ccc4ccccc4c3c2)nc(-c2cccc3oc4ccccc4c23)n1 ; OB(O)c1cccc2oc3ccccc3c12 ; c1ccc2c(c1)ccc1ccc(-c3nc(-c4cccc5oc6ccccc6c45)nc(-c4cccc5oc6ccccc6c45)n3)cc12]

Trz16 (15 g, 32.8 mmol) and dibenzo[b,d]furan-1-ylboronic acid (7.3 g, 34.4 mmol) were added to 300 mL of THF, and the mixture was stirred and refluxed. Then, potassium carbonate (13.6 g, 98.3 mmol) was dissolved in 41 ml of water, added thereto, sufficiently stirred, and then bis(tri-tert-butylphosphine)palladium(0) (0.2 g, 0.3 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 14.3 g of Compound 1-16. (Yield: 74%, MS: [M+H]⁺=590).

### Synthesis Example 1-17

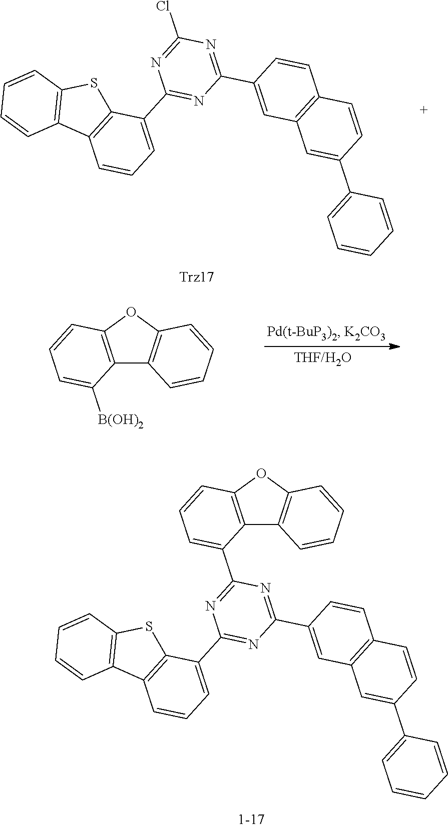 [SMILES: Clc1nc(-c2ccc3ccc(-c4ccccc4)cc3c2)nc(-c2cccc3c2sc2ccccc23)n1 ; OB(O)c1cccc2oc3ccccc3c12 ; c1ccc(-c2ccc3ccc(-c4nc(-c5cccc6c5sc5ccccc56)nc(-c5cccc6oc7ccccc7c56)n4)cc3c2)cc1]

Trz17 (15 g, 30 mmol) and dibenzo[b,d]furan-1-ylboronic acid (6.7 g, 31.5 mmol) were added to 300 mL of THF, and the mixture was stirred and refluxed. Then, potassium carbonate (12.4 g, 90 mmol) was dissolved in 37 ml of water, added thereto, sufficiently stirred, and then bis(tri-tert-butylphosphine)palladium(0) (0.2 g, 0.3 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 14 g of Compound 1-17. (Yield: 74%, MS: [M+H]⁺=632).

### Synthesis Example 1-18

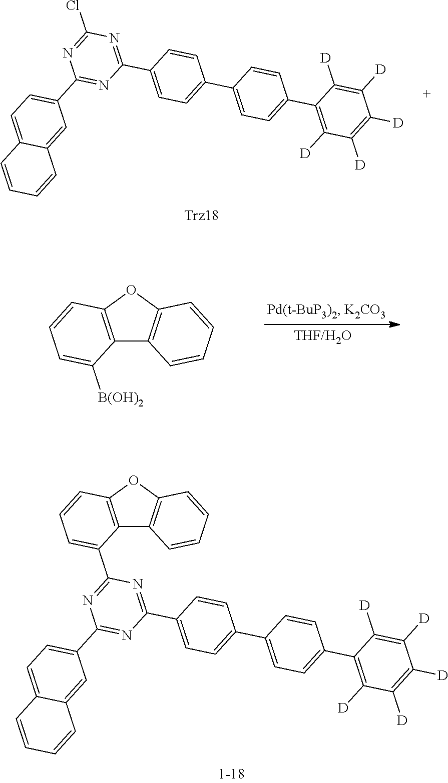 [SMILES: [2H]c1c([2H])c([2H])c(-c2ccc(-c3ccc(-c4nc(Cl)nc(-c5ccc6ccccc6c5)n4)cc3)cc2)c([2H])c1[2H] ; OB(O)c1cccc2oc3ccccc3c12 ; [2H]c1c([2H])c([2H])c(-c2ccc(-c3ccc(-c4nc(-c5ccc6ccccc6c5)nc(-c5cccc6oc7ccccc7c56)n4)cc3)cc2)c([2H])c1[2H]]

Trz17 (15 g, 31.6 mmol) and dibenzo[b,d]furan-1-ylboronic acid (7 g, 33.2 mmol) were added to 300 mL of THF, and the mixture was stirred and refluxed. Then, potassium carbonate (13.1 g, 94.7 mmol) was dissolved in 39 ml of water, added thereto, sufficiently stirred, and then bis(tri-tert-butylphosphine)palladium(0) (0.2 g, 0.3 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 14.2 g of Compound 1-18. (Yield: 74%, MS: [M+H]⁺=607).

### Synthesis Example 1-19

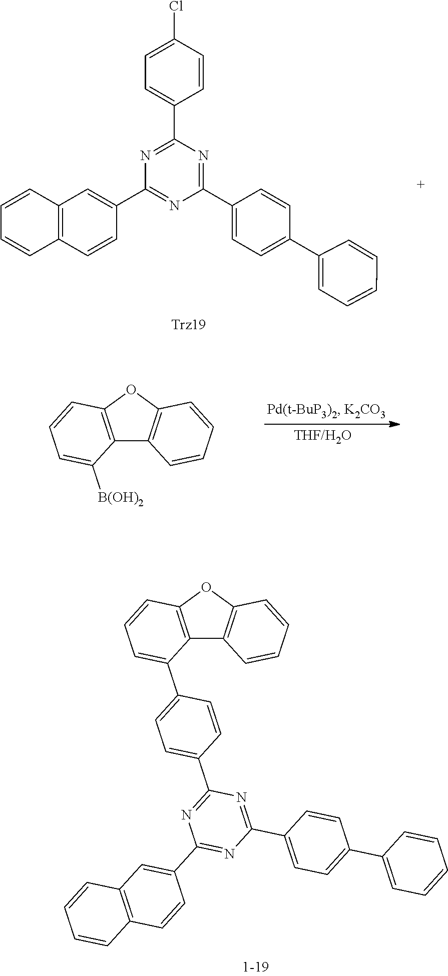 [SMILES: Clc1ccc(-c2nc(-c3ccc(-c4ccccc4)cc3)nc(-c3ccc4ccccc4c3)n2)cc1 ; OB(O)c1cccc2oc3ccccc3c12 ; c1ccc(-c2ccc(-c3nc(-c4ccc(-c5cccc6oc7ccccc7c56)cc4)nc(-c4ccc5ccccc5c4)n3)cc2)cc1]

Trz19 (15 g, 31.9 mmol) and dibenzo[b,d]furan-1-ylboronic acid (7.1 g, 33.5 mmol) were added to 300 mL of THF, and the mixture was stirred and refluxed. Then, potassium carbonate (13.2 g, 95.8 mmol) was dissolved in 40 ml of water, added thereto, sufficiently stirred, and then bis(tri-tert-butylphosphine)palladium(0) (0.2 g, 0.3 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 12.7 g of Compound 1-19. (Yield: 66%, MS: [M+H]⁺=602).

### Synthesis Example 1-20

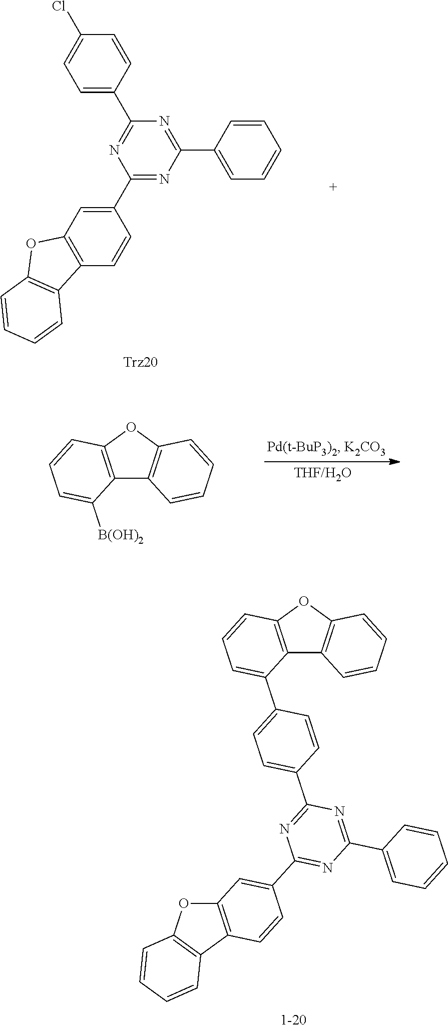 [SMILES: Clc1ccc(-c2nc(-c3ccccc3)nc(-c3ccc4c(c3)oc3ccccc34)n2)cc1 ; OB(O)c1cccc2oc3ccccc3c12 ; c1ccc(-c2nc(-c3ccc(-c4cccc5oc6ccccc6c45)cc3)nc(-c3ccc4c(c3)oc3ccccc34)n2)cc1]

Trz20 (15 g, 34.6 mmol) and dibenzo[b,d]furan-1-ylboronic acid (7.7 g, 36.3 mmol) were added to 300 mL of THF, and the mixture was stirred and refluxed. Then, potassium carbonate (14.3 g, 103.7 mmol) was dissolved in 43 ml of water, added thereto, sufficiently stirred, and then bis(tri-tert-butylphosphine)palladium(0) (0.2 g, 0.3 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.9 g of Compound 1-20. (Yield: 71%, MS: [M+H]⁺=566).

### Synthesis Example 1-21

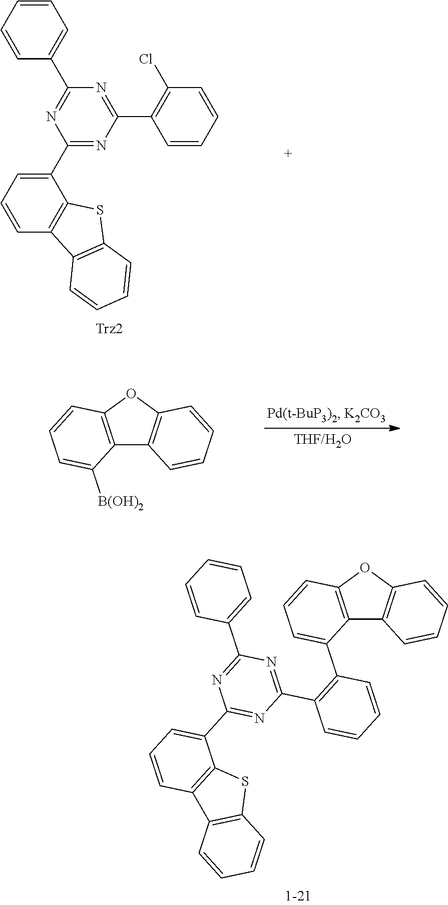 [SMILES: Clc1ccccc1-c1nc(-c2ccccc2)nc(-c2cccc3c2sc2ccccc23)n1 ; OB(O)c1cccc2oc3ccccc3c12 ; c1ccc(-c2nc(-c3ccccc3-c3cccc4oc5ccccc5c34)nc(-c3cccc4c3sc3ccccc34)n2)cc1]

Trz21 (15 g, 33.3 mmol) and dibenzo[b,d]furan-1-ylboronic acid (7.4 g, 35 mmol) were added to 300 mL of THF, and the mixture was stirred and refluxed. Then, potassium carbonate (13.8 g, 100 mmol) was dissolved in 41 ml of water, added thereto, sufficiently stirred, and then bis(tri-tert-butylphosphine)palladium(0) (0.2 g, 0.3 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.9 g of Compound 1-21. (Yield: 72%, MS: [M+H]⁺=582).

### Synthesis Example 1-22

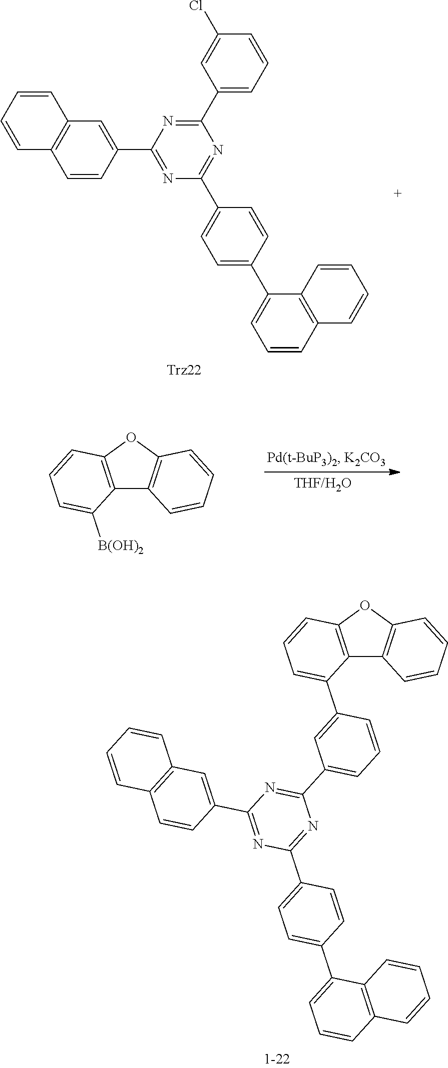 [SMILES: Clc1cccc(-c2nc(-c3ccc(-c4cccc5ccccc45)cc3)nc(-c3ccc4ccccc4c3)n2)c1 ; OB(O)c1cccc2oc3ccccc3c12 ; c1cc(-c2nc(-c3ccc(-c4cccc5ccccc45)cc3)nc(-c3ccc4ccccc4c3)n2)cc(-c2cccc3oc4ccccc4c23)c1]

Trz22 (15 g, 28.8 mmol) and dibenzo[b,d]furan-1-ylboronic acid (6.4 g, 30.3 mmol) were added to 300 mL of THF, and the mixture was stirred and refluxed. Then, potassium carbonate (12 g, 86.5 mmol) was dissolved in 36 ml of water, added thereto, sufficiently stirred, and then bis(tri-tert-butylphosphine)palladium(0) (0.1 g, 0.3 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.3 g of Compound 1-22. (Yield: 71%, MS: [M+H]⁺=652).

### Synthesis Example 1-23

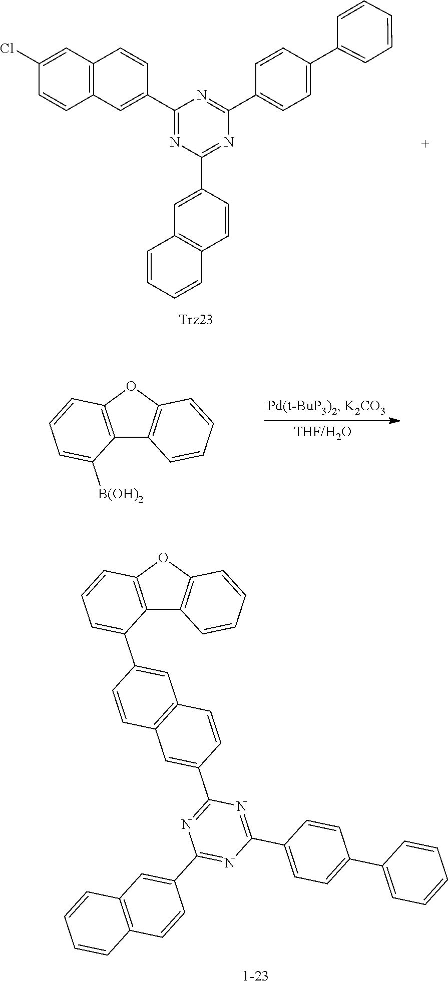 [SMILES: Clc1ccc2cc(-c3nc(-c4ccc(-c5ccccc5)cc4)nc(-c4ccc5ccccc5c4)n3)ccc2c1 ; OB(O)c1cccc2oc3ccccc3c12 ; c1ccc(-c2ccc(-c3nc(-c4ccc5ccccc5c4)nc(-c4ccc5cc(-c6cccc7oc8ccccc8c67)ccc5c4)n3)cc2)cc1]

Trz23 (15 g, 28.8 mmol) and dibenzo[b,d]furan-1-ylboronic acid (6.4 g, 30.3 mmol) were added to 300 mL of THF, and the mixture was stirred and refluxed. Then, potassium carbonate (12 g, 86.5 mmo) was dissolved in 36 ml of water, added thereto, sufficiently stirred, and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.7 g of Compound 1-23. (Yield: 73%, MS: [M+H]⁺=652).

### Synthesis Example 1-24

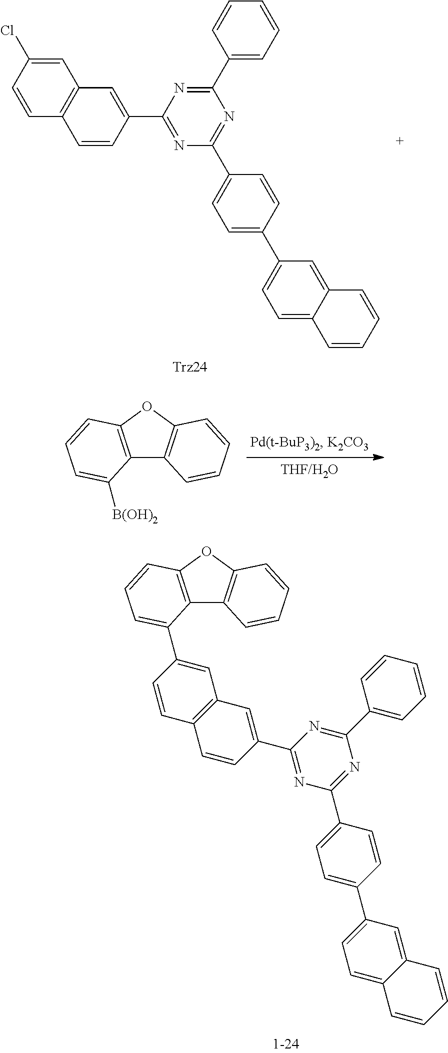 [SMILES: Clc1ccc2ccc(-c3nc(-c4ccccc4)nc(-c4ccc(-c5ccc6ccccc6c5)cc4)n3)cc2c1 ; OB(O)c1cccc2oc3ccccc3c12 ; c1ccc(-c2nc(-c3ccc(-c4ccc5ccccc5c4)cc3)nc(-c3ccc4ccc(-c5cccc6oc7ccccc7c56)cc4c3)n2)cc1]

Trz24 (15 g, 28.8 mmol) and dibenzo[b,d]furan-1-ylboronic acid (6.4 g, 30.3 mmol) were added to 300 mL of THF, and the mixture was stirred and refluxed. Then, potassium carbonate (12 g, 86.5 mmo) was dissolved in 36 ml of water, added thereto, sufficiently stirred, and then bis(tri-tert-butylphosphine)palladium(0) (0.1 g, 0.3 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 12.6 g of Compound 1-24. (Yield: 67%, MS: [M+H]⁺=652).

### Synthesis Example 1-25

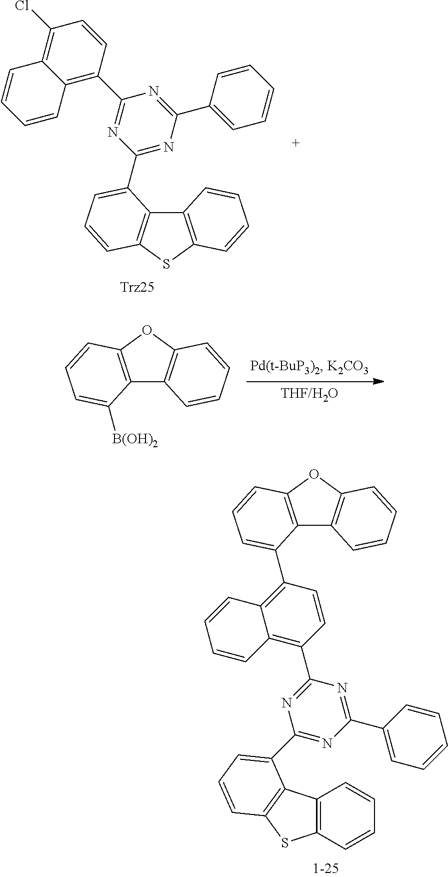 [SMILES: Clc1ccc(-c2nc(-c3ccccc3)nc(-c3cccc4sc5ccccc5c34)n2)c2ccccc12 ; OB(O)c1cccc2oc3ccccc3c12 ; c1ccc(-c2nc(-c3ccc(-c4cccc5oc6ccccc6c45)c4ccccc34)nc(-c3cccc4sc5ccccc5c34)n2)cc1]

Trz25 (15 g, 30 mmol) and dibenzo[b,d]furan-1-ylboronic acid (6.7 g, 31.5 mmol) were added to 300 mL of THF, and the mixture was stirred and refluxed. Then, potassium carbonate (12.4 g, 90 mmol) was dissolved in 37 ml of water, added thereto, sufficiently stirred, and then bis(tri-tert-butylphosphine)palladium(0) (0.2 g, 0.3 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 14.2 g of Compound 1-25. (Yield: 75%, MS: [M+H]⁺=632).

### Synthesis Example 1-26

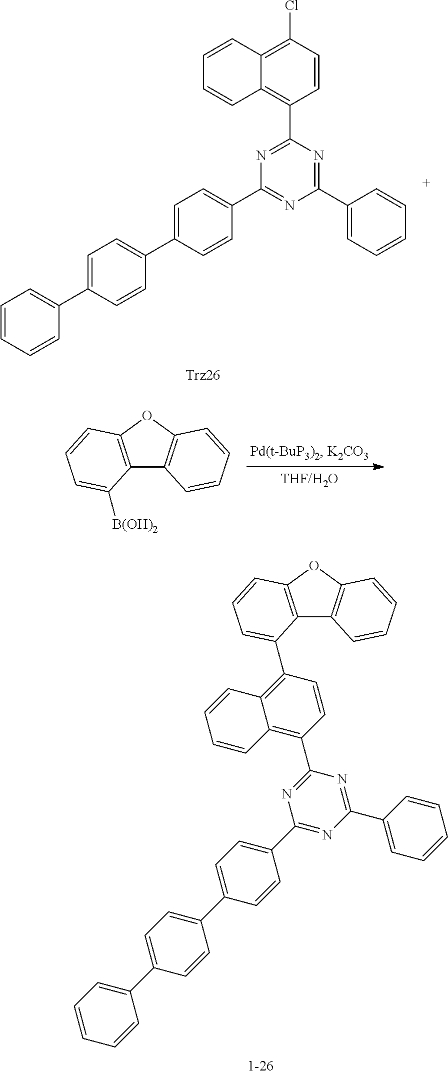 [SMILES: Clc1ccc(-c2nc(-c3ccccc3)nc(-c3ccc(-c4ccc(-c5ccccc5)cc4)cc3)n2)c2ccccc12 ; OB(O)c1cccc2oc3ccccc3c12 ; c1ccc(-c2ccc(-c3ccc(-c4nc(-c5ccccc5)nc(-c5ccc(-c6cccc7oc8ccccc8c67)c6ccccc56)n4)cc3)cc2)cc1]

Trz26 (15 g, 27.5 mmol) and dibenzo[b,d]furan-1-ylboronic acid (6.1 g, 28.8 mmol) were added to 300 mL of THF, and the mixture was stirred and refluxed. Then, potassium carbonate (11.4 g, 82.4 mmol) was dissolved in 34 ml of water, added thereto, sufficiently stirred, and then bis(tri-tert-butylphosphine)palladium(0) (0.1 g, 0.3 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 14 g of Compound 1-26. (Yield: 75%, MS: [M+H]⁺=678).

### Synthesis Example 1-27

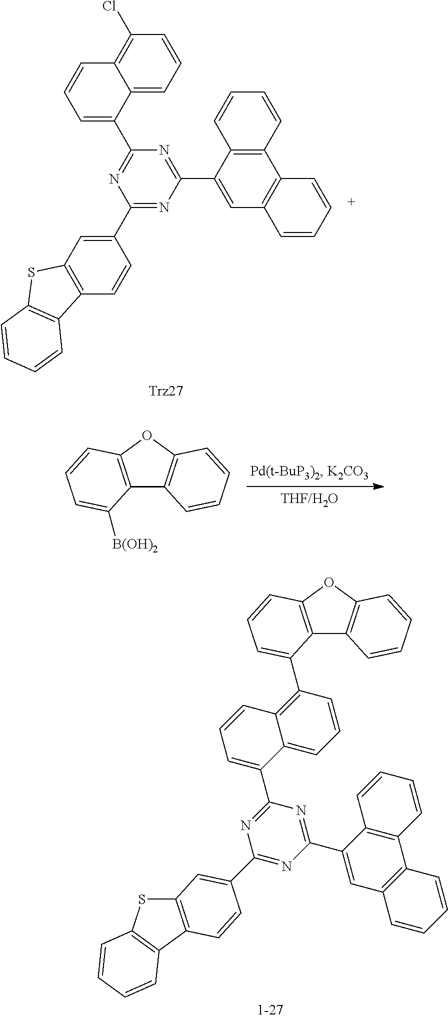 [SMILES: Clc1cccc2c(-c3nc(-c4ccc5c(c4)sc4ccccc45)nc(-c4cc5ccccc5c5ccccc45)n3)cccc12 ; OB(O)c1cccc2oc3ccccc3c12 ; c1ccc2c(c1)cc(-c1nc(-c3ccc4c(c3)sc3ccccc34)nc(-c3cccc4c(-c5cccc6oc7ccccc7c56)cccc34)n1)c1ccccc12]

Trz27 (15 g, 25 mmol) and dibenzo[b,d]furan-1-ylboronic acid (5.6 g, 26.2 mmol) were added to 300 mL of THF, and the mixture was stirred and refluxed. Then, potassium carbonate (10.4 g, 75 mmol) was dissolved in 31 ml of water, added thereto, sufficiently stirred, and then bis(tri-tert-butylphosphine)palladium(0) (0.1 g, 0.2 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 12.6 g of Compound 1-27. (Yield: 69%, MS: [M+H]⁺=732).

### Synthesis Example 1-28

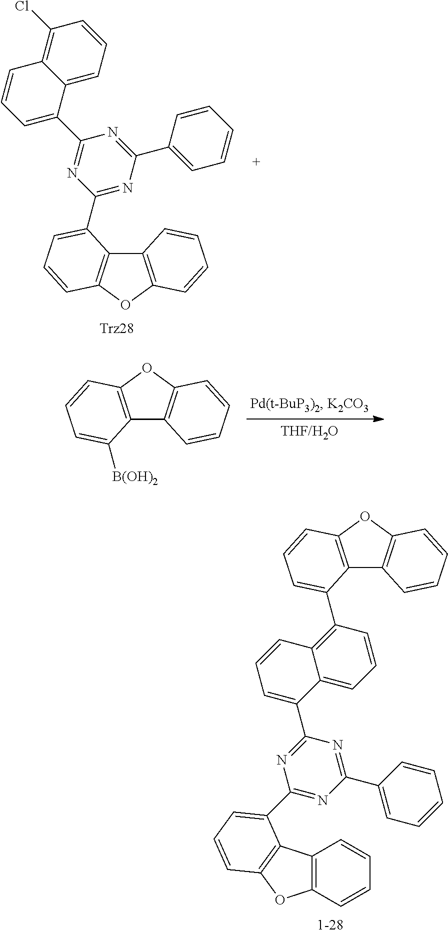 [SMILES: Clc1cccc2c(-c3nc(-c4ccccc4)nc(-c4cccc5oc6ccccc6c45)n3)cccc12 ; OB(O)c1cccc2oc3ccccc3c12 ; c1ccc(-c2nc(-c3cccc4c(-c5cccc6oc7ccccc7c56)cccc34)nc(-c3cccc4oc5ccccc5c34)n2)cc1]

Trz28 (15 g, 31 mmol) and dibenzo[b,d]furan-1-ylboronic acid (6.9 g, 32.5 mmol) were added to 300 mL of THF, and the mixture was stirred and refluxed. Then, potassium carbonate (12.9 g, 93 mmol) was dissolved in 39 ml of water, added thereto, sufficiently stirred, and then bis(tri-tert-butylphosphine)palladium(0) (0.2 g, 0.3 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13 g of Compound 1-28. (Yield: 68%, MS: [M+H]⁺=616).

### Synthesis Example 1-29

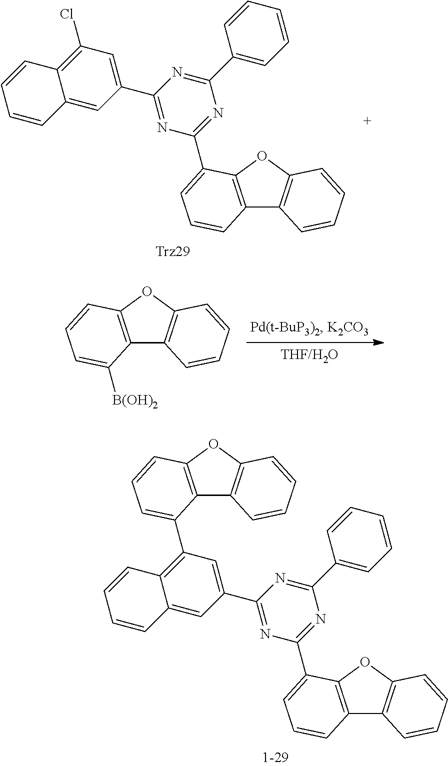 [SMILES: Clc1cc(-c2nc(-c3ccccc3)nc(-c3cccc4c3oc3ccccc34)n2)cc2ccccc12 ; OB(O)c1cccc2oc3ccccc3c12 ; c1ccc(-c2nc(-c3cc(-c4cccc5oc6ccccc6c45)c4ccccc4c3)nc(-c3cccc4c3oc3ccccc34)n2)cc1]

Trz29 (15 g, 31 mmol) and dibenzo[b,d]furan-1-ylboronic acid (6.9 g, 32.5 mmol) were added to 300 mL of THF, and the mixture was stirred and refluxed. Then, potassium carbonate (12.9 g, 93 mmol) was dissolved in 39 ml of water, added thereto, sufficiently stirred, and then bis(tri-tert-butylphosphine)palladium(0) (0.2 g, 0.3 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.3 g of Compound 1-29. (Yield: 70%, MS: [M+H]⁺=616).

### Synthesis Example 1-30

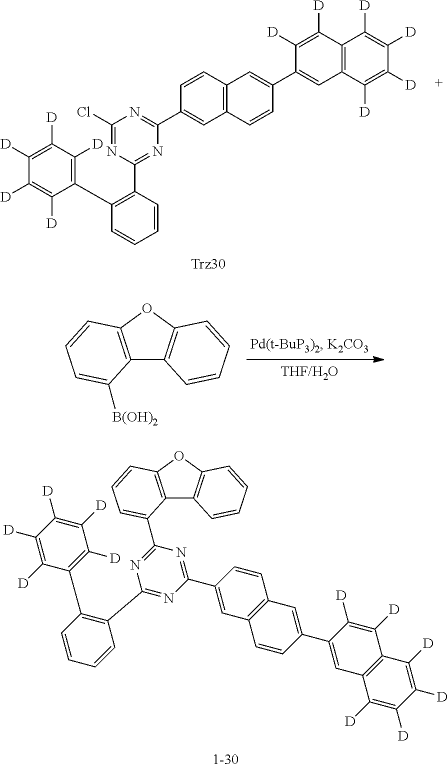 [SMILES: [2H]c1c([2H])c([2H])c(-c2ccccc2-c2nc(Cl)nc(-c3ccc4cc(-c5cc6c([2H])c([2H])c([2H])c([2H])c6c([2H])c5[2H])ccc4c3)n2)c([2H])c1[2H] ; OB(O)c1cccc2oc3ccccc3c12 ; [2H]c1c([2H])c([2H])c(-c2ccccc2-c2nc(-c3ccc4cc(-c5cc6c([2H])c([2H])c([2H])c([2H])c6c([2H])c5[2H])ccc4c3)nc(-c3cccc4oc5ccccc5c34)n2)c([2H])c1[2H]]

Trz30 (15 g, 28.2 mmol) and dibenzo[b,d]furan-1-ylboronic acid (6.3 g, 29.7 mmol) were added to 300 mL of THF, and the mixture was stirred and refluxed. Then, potassium carbonate (11.7 g, 84.7 mmol) was dissolved in 35 ml of water, added thereto, sufficiently stirred, and then bis(tri-tert-butylphosphine)palladium(0) (0.1 g, 0.3 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 12.9 g of Compound 1-30. (Yield: 69%, MS: [M+H]⁺=663).

### Synthesis Example 1-31

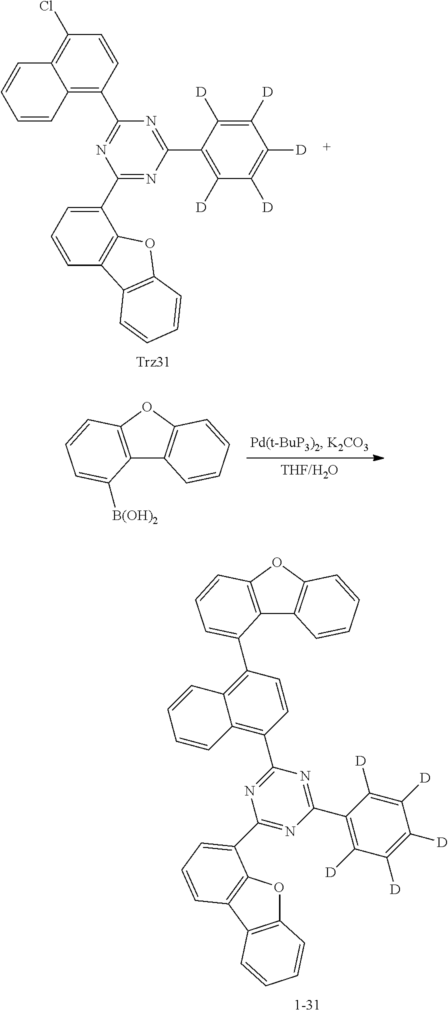 [SMILES: [2H]c1c([2H])c([2H])c(-c2nc(-c3ccc(Cl)c4ccccc34)nc(-c3cccc4c3oc3ccccc34)n2)c([2H])c1[2H] ; OB(O)c1cccc2oc3ccccc3c12 ; [2H]c1c([2H])c([2H])c(-c2nc(-c3ccc(-c4cccc5oc6ccccc6c45)c4ccccc34)nc(-c3cccc4c3oc3ccccc34)n2)c([2H])c1[2H]]

Trz31 (15 g, 30.7 mmol) and dibenzo[b,d]furan-1-ylboronic acid (6.8 g, 32.2 mmol) were added to 300 mL of THF, and the mixture was stirred and refluxed. Then, potassium carbonate (12.7 g, 92 mmol) was dissolved in 38 ml of water, added thereto, sufficiently stirred, and then bis(tri-tert-butylphosphine)palladium(0) (0.2 g, 0.3 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 14.3 g of Compound 1-31. (Yield: 75%, MS: [M+H]⁺=621).

### Synthesis Example 1-32

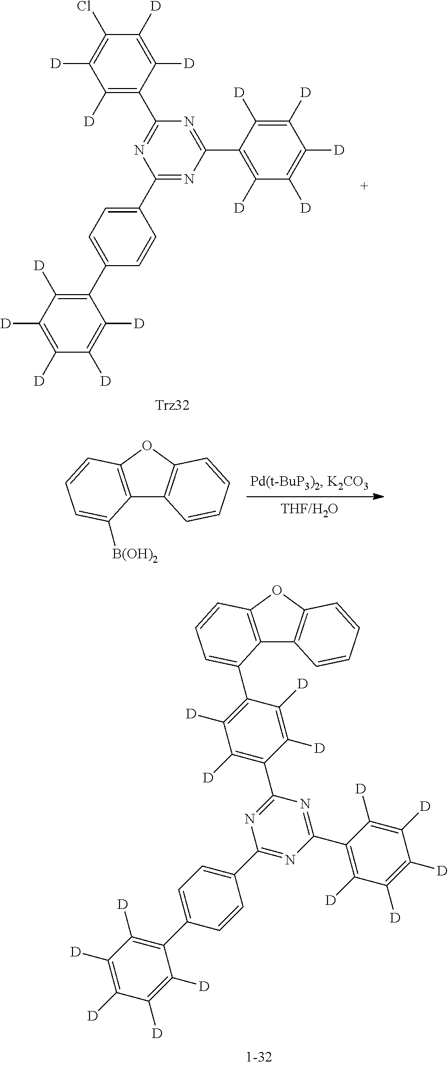 [SMILES: [2H]c1c([2H])c([2H])c(-c2ccc(-c3nc(-c4c([2H])c([2H])c([2H])c([2H])c4[2H])nc(-c4c([2H])c([2H])c(Cl)c([2H])c4[2H])n3)cc2)c([2H])c1[2H] ; OB(O)c1cccc2oc3ccccc3c12 ; [2H]c1c([2H])c([2H])c(-c2ccc(-c3nc(-c4c([2H])c([2H])c([2H])c([2H])c4[2H])nc(-c4c([2H])c([2H])c(-c5cccc6oc7ccccc7c56)c([2H])c4[2H])n3)cc2)c([2H])c1[2H]]

Trz32 (15 g, 34.6 mmol) and dibenzo[b,d]furan-1-ylboronic acid (7.7 g, 36.3 mmol) were added to 300 mL of THF, and the mixture was stirred and refluxed. Then, potassium carbonate (14.3 g, 103.7 mmol) was dissolved in 43 ml of water, added thereto, sufficiently stirred, and then bis(tri-tert-butylphosphine)palladium(0) (0.2 g, 0.3 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.9 g of Compound 1-32. (Yield: 71%, MS: [M+H]⁺=566).

### Synthesis Example 1-33

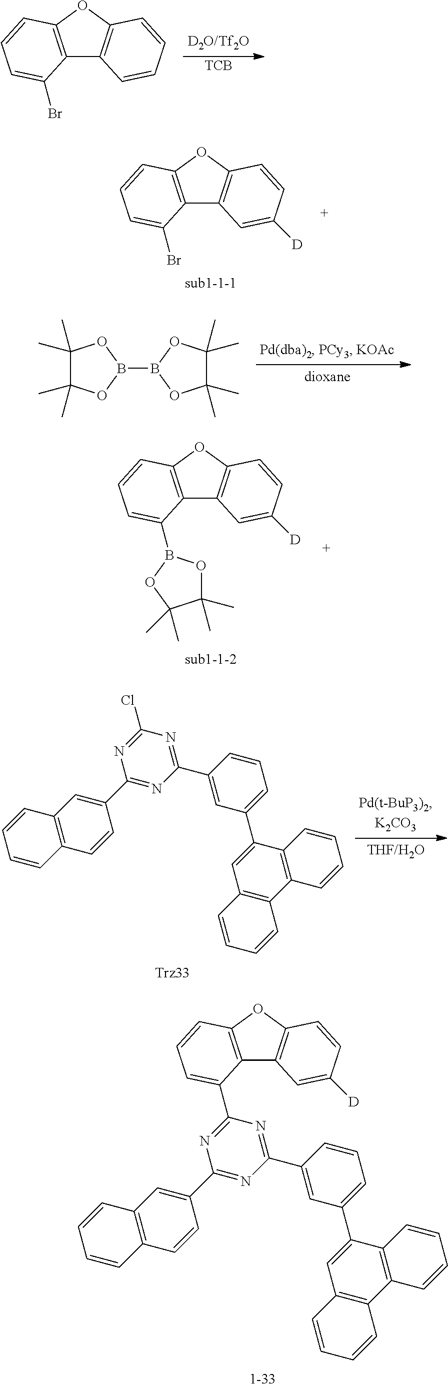

Trifluoromethanesulfonic anhydride (24 g, 85 mmol) and deuterium oxide (8.5 g, 424.9 mmol) were added at 0° C., and the mixture was stirred for 5 hours to prepare a solution. 1-Bromodibenzo[b,d]furan (15 g, 60.7 mmol) was added to 120 mL of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating up to 140° C. and then keeping that temperature. After the reaction for 5 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.7 g of sub1-1-1. (Yield: 38%, MS: [M+H]⁺=248)

sub1-1-1 (15 g, 60.5 mmol) and bis(pinacolato)diboron (16.9 g, 66.5 mmol) were added to 300 ml of 1,4-dioxane, and the mixture was stirred under reflux. Then, potassium acetate (8.9 g, 90.7 mmol) was added thereto, sufficiently stirred, and then bis(dibenzylideneacetone)palladium(0) (1 g, 1.8 mmol) and tricyclohexylphosphine (1 g, 3.6 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by a silica gel column chromatography to prepare 13.4 g of sub1-1-2. (Yield: 75%, MS: [M+H]⁺=296)

sub1-2-2 (15 g, 50.8 mmol) and Trz33 (26.4 g, 53.4 mmol) were added to 300 ml of THF, and the mixture was stirred and refluxed. Then, potassium carbonate (21.1 g, 152.5 mmol) was dissolved in 63 ml of water, added thereto, sufficiently stirred, and then bis(tri-tert-butylphosphine)palladium(0) (0.3 g, 0.5 mmol) was added. After the reaction for 3 hours, the reaction mixture was cooled to room temperature, and then the organic layer and the aqueous layer were separated, and the organic layer was distilled. This was dissolved again in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 21 g of Compound 1-33. (Yield: 66%, MS: [M+H]⁺=627)

### Synthesis Example 1-34

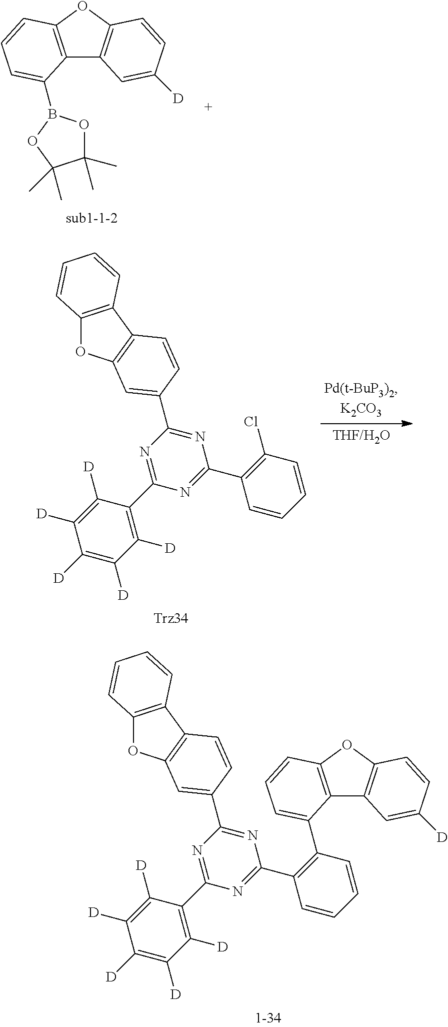 [SMILES: [2H]c1ccc2oc3cccc(B4OC(C)(C)C(C)(C)O4)c3c2c1 ; [2H]c1c([2H])c([2H])c(-c2nc(-c3ccc4c(c3)oc3ccccc34)nc(-c3ccccc3Cl)n2)c([2H])c1[2H] ; [2H]c1ccc2oc3cccc(-c4ccccc4-c4nc(-c5ccc6c(c5)oc5ccccc56)nc(-c5c([2H])c([2H])c([2H])c([2H])c5[2H])n4)c3c2c1]

sub1-2-2 (15 g, 50.8 mmol) and Trz34 (23.4 g, 53.4 mmol) were added to 300 mL of THF, and the mixture was stirred and refluxed. Then, potassium carbonate (21.1 g, 152.5 mmol) was dissolved in 63 ml of water, added thereto, sufficiently stirred, and then bis(tri-tert-butylphosphine)palladium(0) (0.3 g, 0.5 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 19.4 g of Compound 1-34. (Yield: 67%, MS: [M+H]⁺=572).

### Synthesis Example 1-35

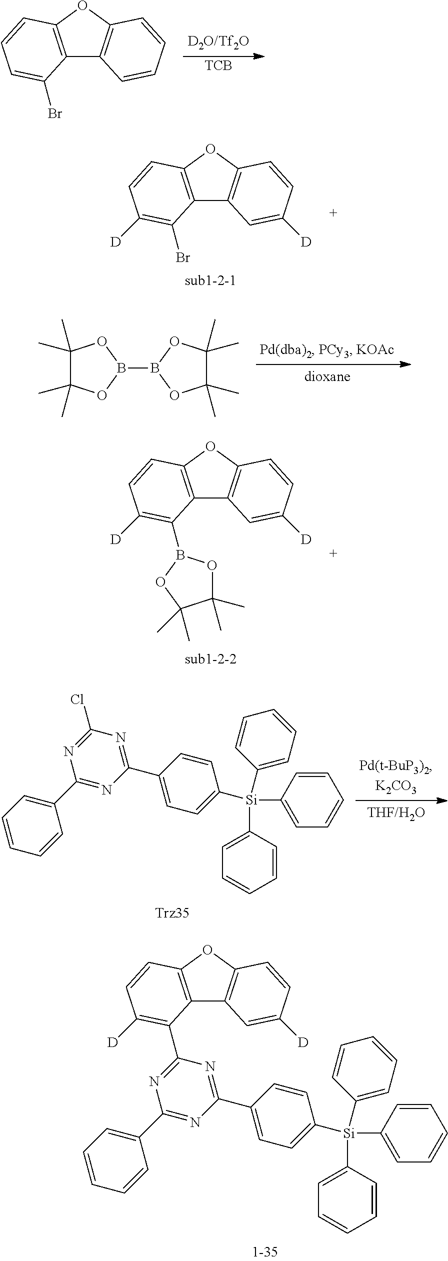

Trifluoromethanesulfonic anhydride (48 g, 170 mmol) and deuterium oxide (17 g, 849.9 mmol) were added at 0° C., and the mixture was stirred for 5 hours to prepare a solution. 1-Bromodibenzo[b,d]furan (15 g, 60.7 mmol) was added to 120 mL of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating up to 140° C. and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6 g of sub1-2-1. (Yield: 40%, MS: [M+H]⁺=249)

sub1-2-1 (15 g, 60.2 mmol) and bis(pinacolato)diboron (16.8 g, 66.2 mmol) were added to 300 ml of 1,4-dioxane, and the mixture was stirred under reflux. Then, potassium acetate (8.9 g, 90.3 mmol) was added thereto, sufficiently stirred, and then bis(dibenzylideneacetone)palladium(0) (1 g, 1.8 mmol) and tricyclohexylphosphine (1 g, 3.6 mmol) were added. After the reaction for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by a silica gel column chromatography to prepare 12.5 g of sub1-2-2. (Yield: 70%, MS: [M+H]⁺=297)

sub1-2-2 (15 g, 50.6 mmol) and Trz35 (28 g, 53.2 mmol) were added to 300 ml of THF, and the mixture was stirred and refluxed. Then, potassium carbonate (21 g, 151.9 mmol) was dissolved in 63 ml of water, added thereto, sufficiently stirred, and then bis(tri-tert-butylphosphine)palladium(0) (0.3 g, 0.5 mmol) was added. After the reaction for 4 hours, the reaction mixture was cooled to room temperature, and then the organic layer and the aqueous layer were separated, and the organic layer was distilled. This was dissolved again in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 23.4 g of Compound 1-35. (Yield: 70%, MS: [M+H]⁺=660)

### Synthesis Example 1-36

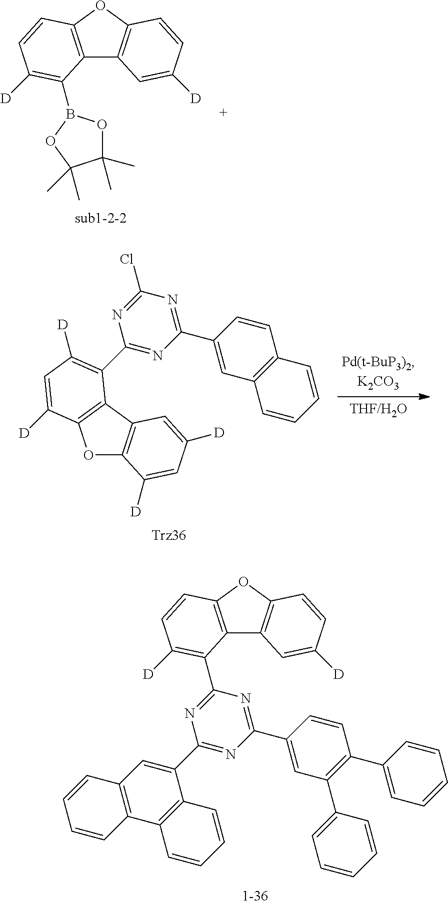 [SMILES: [2H]c1ccc2oc3ccc([2H])c(B4OC(C)(C)C(C)(C)O4)c3c2c1 ; [2H]c1cc([2H])c2oc3c([2H])cc([2H])c(-c4nc(Cl)nc(-c5ccc6ccccc6c5)n4)c3c2c1 ; [2H]c1ccc2oc3ccc([2H])c(-c4nc(-c5ccc(-c6ccccc6)c(-c6ccccc6)c5)nc(-c5cc6ccccc6c6ccccc56)n4)c3c2c1]

sub1-2-2 (15 g, 50.6 mmol) and Trz36 (21.9 g, 53.2 mmol) were added to 300 mL of THF, and the mixture was stirred and refluxed. Then, potassium carbonate (21 g, 151.9 mmol) was dissolved in 63 ml of water, added thereto, sufficiently stirred, and then bis(tri-tert-butylphosphine)palladium(0) (0.3 g, 0.5 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 22.5 g of Compound 1-36. (Yield: 68%, MS: [M+H]⁺=654).

### Synthesis Example 1-37

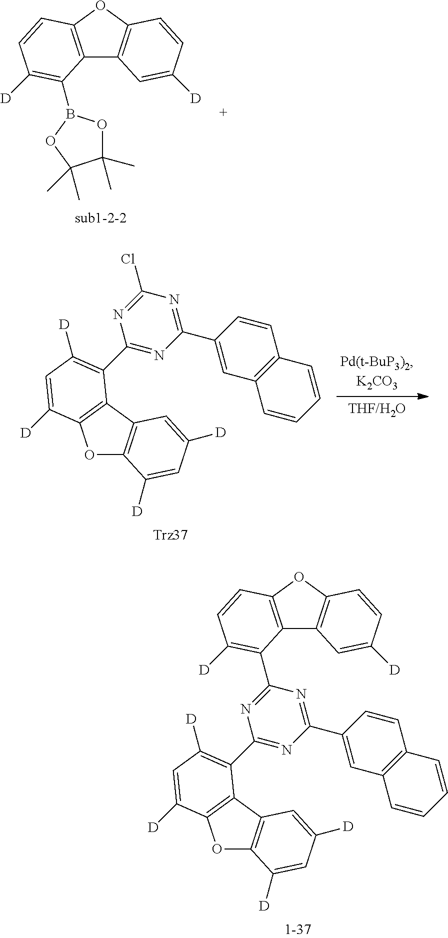 [SMILES: [2H]c1ccc2oc3ccc([2H])c(B4OC(C)(C)C(C)(C)O4)c3c2c1 ; [2H]c1cc([2H])c2oc3c([2H])cc([2H])c(-c4nc(Cl)nc(-c5ccc6ccccc6c5)n4)c3c2c1 ; [2H]c1ccc2oc3ccc([2H])c(-c4nc(-c5ccc6ccccc6c5)nc(-c5c([2H])cc([2H])c6oc7c([2H])cc([2H])cc7c56)n4)c3c2c1]

sub1-2-2 (15 g, 50.6 mmol) and Trz37 (21.9 g, 53.2 mmol) were added to 300 mL of THF, and the mixture was stirred and refluxed. Then, potassium carbonate (21 g, 151.9 mmol) was dissolved in 63 ml of water, added thereto, sufficiently stirred, and then bis(tri-tert-butylphosphine)palladium(0) (0.3 g, 0.5 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 17.9 g of Compound 1-37. (Yield: 65%, MS: [M+H]⁺=546).

### Synthesis Example 1-38

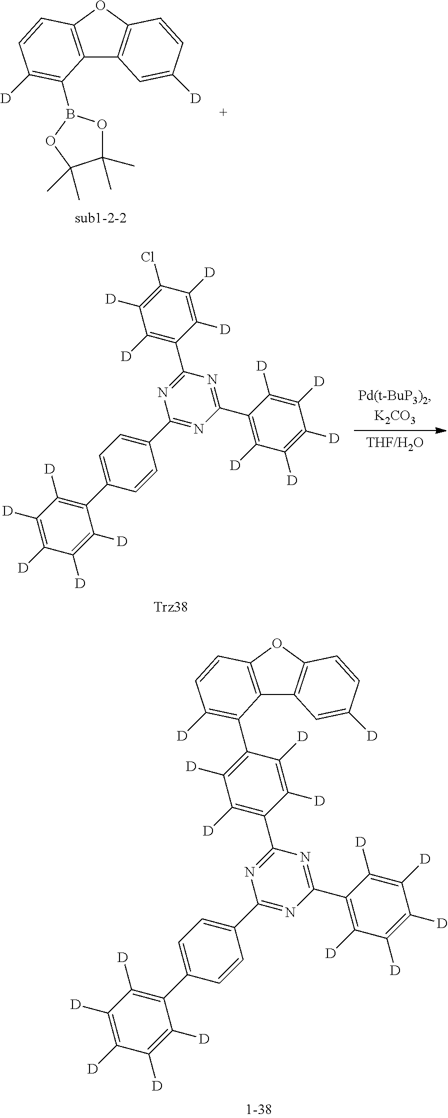 [SMILES: [2H]c1ccc2oc3ccc([2H])c(B4OC(C)(C)C(C)(C)O4)c3c2c1 ; [2H]c1c([2H])c([2H])c(-c2ccc(-c3nc(-c4c([2H])c([2H])c([2H])c([2H])c4[2H])nc(-c4c([2H])c([2H])c(Cl)c([2H])c4[2H])n3)cc2)c([2H])c1[2H] ; [2H]c1ccc2oc3ccc([2H])c(-c4c([2H])c([2H])c(-c5nc(-c6ccc(-c7c([2H])c([2H])c([2H])c([2H])c7[2H])cc6)nc(-c6c([2H])c([2H])c([2H])c([2H])c6[2H])n5)c([2H])c4[2H])c3c2c1]

sub1-2-2 (15 g, 50.6 mmol) and Trz36 (23.1 g, 53.2 mmol) were added to 300 mL of THF, and the mixture was stirred and refluxed. Then, potassium carbonate (21 g, 151.9 mmol) was dissolved in 63 ml of water, added thereto, sufficiently stirred, and then bis(tri-tert-butylphosphine)palladium(0) (0.3 g, 0.5 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 19 g of Compound 1-38. (Yield: 66%, MS: [M+H]⁺=568).

### Synthesis Example 1-39

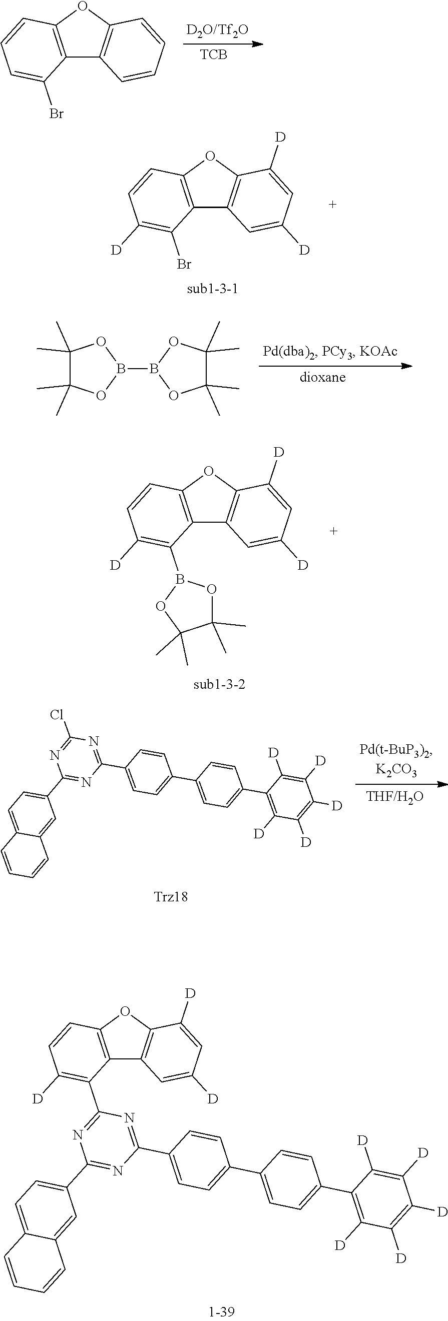

Trifluoromethanesulfonic anhydride (71.9 g, 255 mmol) and deuterium oxide (25.5 g, 1274.8 mmol) were added at 0° C., and the mixture was stirred for 5 hours to prepare a solution. 1-Bromodibenzo[b,d]furan (15 g, 60.7 mmol) was added to 120 mL of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating up to 140° C. and then keeping that temperature. After the reaction for 14 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.3 g of sub1-3-1. (Yield: 42%, MS: [M+H]⁺=250)

sub1-3-1 (15 g, 60 mmol) and bis(pinacolato)diboron (16.8 g, 66 mmol) were added to 300 ml of 1,4-dioxane, and the mixture was stirred under reflux. Then, potassium acetate (8.8 g, 90 mmol) was added thereto, sufficiently stirred, and then bis(dibenzylideneacetone)palladium(0) (1 g, 1.8 mmol) and tricyclohexylphosphine (1 g, 3.6 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by a silica gel column chromatography to prepare 11.4 g of sub1-3-2. (Yield: 64%, MS: [M+H]⁺=298)

sub1-3-2 (15 g, 50.5 mmol) and Trz18 (25.2 g, 53 mmol) were added to 300 ml of THF, and the mixture was stirred and refluxed. Then, potassium carbonate (20.9 g, 151.4 mmol) was dissolved in 63 ml of water, added thereto, sufficiently stirred, and then bis(tri-tert-butylphosphine)palladium(0) (0.3 g, 0.5 mmol) was added. After the reaction for 3 hours, the reaction mixture was cooled to room temperature, and then the organic layer and the aqueous layer were separated, and the organic layer was distilled. This was dissolved again in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 23.1 g of Compound 1-39. (Yield: 75%, MS: [M+H]⁺=610)

### Synthesis Example 1-40

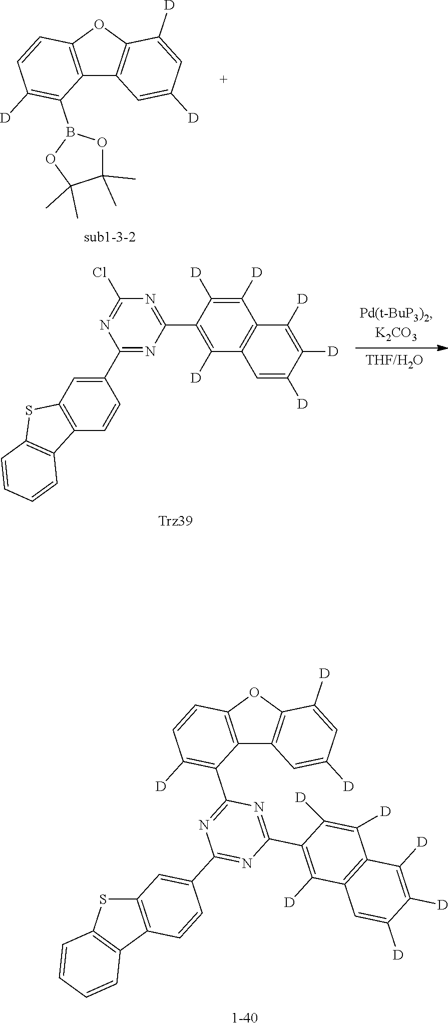 [SMILES: [2H]c1cc([2H])c2oc3ccc([2H])c(B4OC(C)(C)C(C)(C)O4)c3c2c1 ; [2H]c1cc2c([2H])c(-c3nc(Cl)nc(-c4ccc5c(c4)sc4ccccc45)n3)c([2H])c([2H])c2c([2H])c1[2H] ; [2H]c1cc([2H])c2oc3ccc([2H])c(-c4nc(-c5ccc6c(c5)sc5ccccc56)nc(-c5c([2H])c([2H])c6c([2H])c([2H])c([2H])cc6c5[2H])n4)c3c2c1]

sub1-3-2 (15 g, 50.5 mmol) and Trz39 (22.8 g, 53 mmol) were added to 300 mL of THF, and the mixture was stirred and refluxed. Then, potassium carbonate (20.9 g, 151.4 mmol) was dissolved in 63 ml of water, added thereto, sufficiently stirred, and then bis(tri-tert-butylphosphine)palladium(0) (0.3 g, 0.5 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 18.5 g of Compound 1-40. (Yield: 65%, MS: [M+H]⁺=565).

### Synthesis Example 1-41

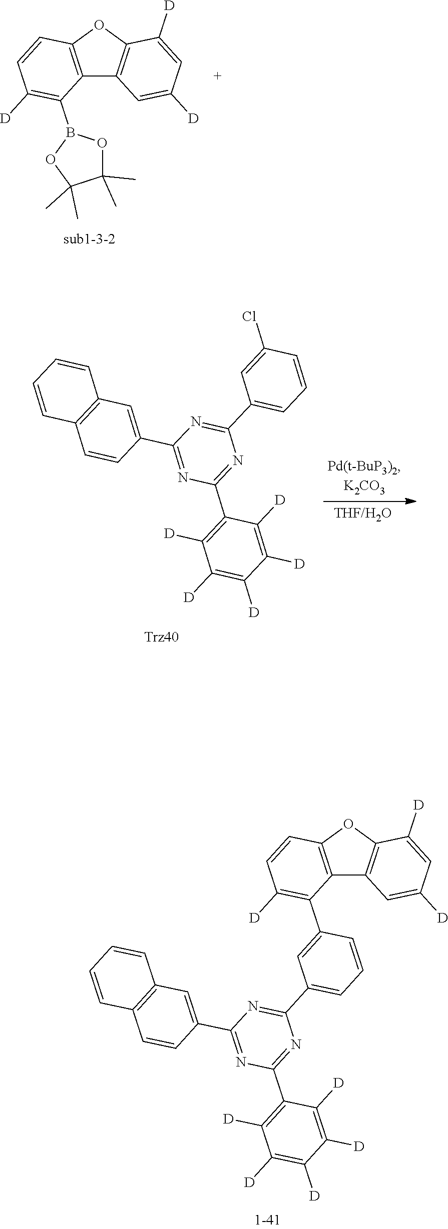 [SMILES: [2H]c1cc([2H])c2oc3ccc([2H])c(B4OC(C)(C)C(C)(C)O4)c3c2c1 ; [2H]c1c([2H])c([2H])c(-c2nc(-c3cccc(Cl)c3)nc(-c3ccc4ccccc4c3)n2)c([2H])c1[2H] ; [2H]c1cc([2H])c2oc3ccc([2H])c(-c4cccc(-c5nc(-c6ccc7ccccc7c6)nc(-c6c([2H])c([2H])c([2H])c([2H])c6[2H])n5)c4)c3c2c1]

sub1-3-2 (15 g, 50.5 mmol) and Trz39 (21.1 g, 53 mmol) were added to 300 mL of THF, and the mixture was stirred and refluxed. Then, potassium carbonate (20.9 g, 151.4 mmol) was dissolved in 63 ml of water, added thereto, sufficiently stirred, and then bis(tri-tert-butylphosphine)palladium(0) (0.3 g, 0.5 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 17.8 g of Compound 1-41. (Yield: 66%, MS: [M+H]⁺=534).

### Synthesis Example 1-42

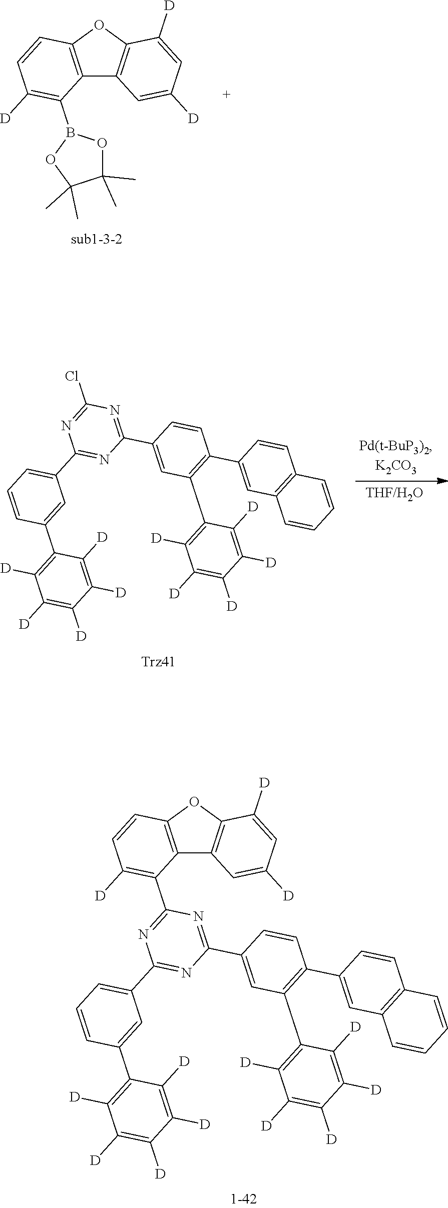 [SMILES: [2H]c1cc([2H])c2oc3ccc([2H])c(B4OC(C)(C)C(C)(C)O4)c3c2c1 ; [2H]c1c([2H])c([2H])c(-c2cccc(-c3nc(Cl)nc(-c4ccc(-c5ccc6ccccc6c5)c(-c5c([2H])c([2H])c([2H])c([2H])c5[2H])c4)n3)c2)c([2H])c1[2H] ; [2H]c1cc([2H])c2oc3ccc([2H])c(-c4nc(-c5cccc(-c6c([2H])c([2H])c([2H])c([2H])c6[2H])c5)nc(-c5ccc(-c6ccc7ccccc7c6)c(-c6c([2H])c([2H])c([2H])c([2H])c6[2H])c5)n4)c3c2c1]

sub1-3-2 (15 g, 50.5 mmol) and Trz41 (29.5 g, 53 mmol) were added to 300 mL of THF, and the mixture was stirred and refluxed. Then, potassium carbonate (20.9 g, 151.4 mmol) was dissolved in 63 ml of water, added thereto, sufficiently stirred, and then bis(tri-tert-butylphosphine)palladium(0) (0.3 g, 0.5 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 24.4 g of Compound 1-42. (Yield: 70%, MS: [M+H]⁺=691).

### Synthesis Example 1-43

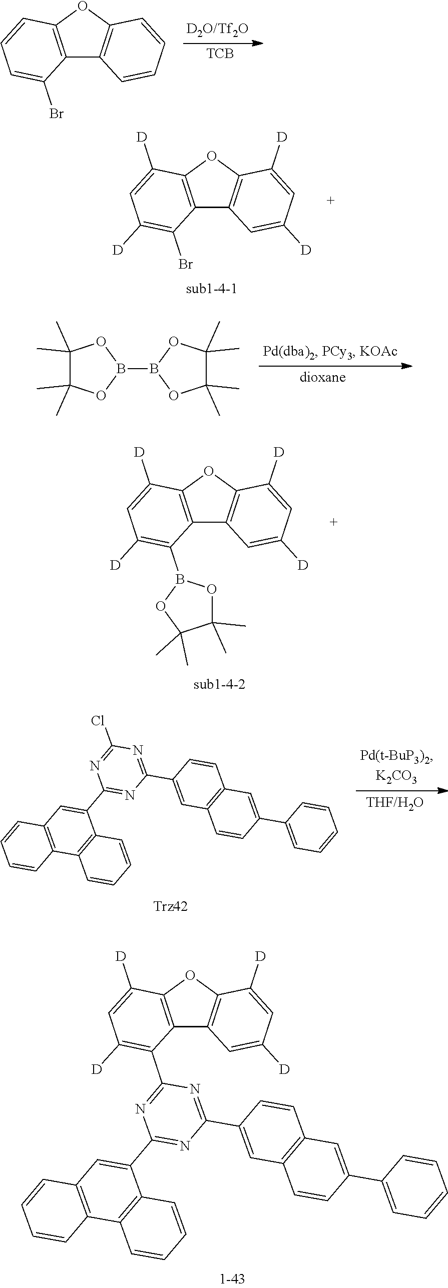

Trifluoromethanesulfonic anhydride (95.9 g, 340 mmol) and deuterium oxide (34 g, 1699.8 mmol) were added at 0° C., and the mixture was stirred for 5 hours to prepare a solution. 1-Bromodibenzo[b,d]furan (15 g, 60.7 mmol) was added to 120 mL of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating up to 140° C. and then keeping that temperature. After the reaction for 20 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.6 g of sub1-4-1. (Yield: 37%, MS: [M+H]⁺=251)

sub1-4-1 (15 g, 59.7 mmol) and bis(pinacolato)diboron (16.7 g, 65.7 mmol) were added to 300 ml of 1,4-dioxane, and the mixture was stirred under reflux. Then, potassium acetate (8.8 g, 89.6 mmol) was added thereto, sufficiently stirred, and then bis(dibenzylideneacetone)palladium(0) (1 g, 1.8 mmol) and tricyclohexylphosphine (1 g, 3.6 mmol) were added. After the reaction for 5 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by a silica gel column chromatography to prepare 12.5 g of sub1-4-2. (Yield: 70%, MS: [M+H]⁺=299)

sub1-4-2 (15 g, 50.3 mmol) and Trz42 (26.1 g, 52.8 mmol) were added to 300 ml of THF, and the mixture was stirred and refluxed. Then, potassium carbonate (20.9 g, 150.9 mmol) was dissolved in 63 ml of water, added thereto, sufficiently stirred, and then bis(tri-tert-butylphosphine)palladium(0) (0.3 g, 0.5 mmol) was added. After the reaction for 4 hours, the reaction mixture was cooled to room temperature, and then the organic layer and the aqueous layer were separated, and the organic layer was distilled. This was dissolved again in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 21.5 g of Compound 1-43. (Yield: 68%, MS: [M+H]⁺=631)

### Synthesis Example 1-44

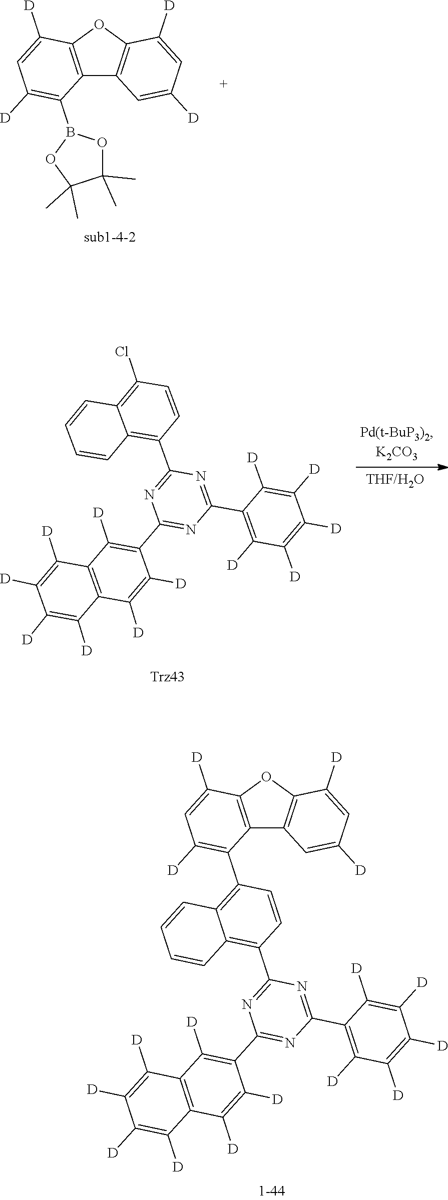 [SMILES: [2H]c1cc([2H])c2oc3c([2H])cc([2H])c(B4OC(C)(C)C(C)(C)O4)c3c2c1 ; [2H]c1c([2H])c([2H])c(-c2nc(-c3c([2H])c([2H])c4c([2H])c([2H])c([2H])c([2H])c4c3[2H])nc(-c3ccc(Cl)c4ccccc34)n2)c([2H])c1[2H] ; [2H]c1cc([2H])c2oc3c([2H])cc([2H])c(-c4ccc(-c5nc(-c6c([2H])c([2H])c([2H])c([2H])c6[2H])nc(-c6c([2H])c([2H])c7c([2H])c([2H])c([2H])c([2H])c7c6[2H])n5)c5ccccc45)c3c2c1]

sub1-4-2 (15 g, 50.3 mmol) and Trz43 (24.1 g, 52.8 mmol) were added to 300 mL of THF, and the mixture was stirred and refluxed. Then, potassium carbonate (20.9 g, 150.9 mmol) was dissolved in 63 ml of water, added thereto, sufficiently stirred, and then bis(tri-tert-butylphosphine)palladium(0) (0.3 g, 0.5 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 20.2 g of Compound 1-44. (Yield: 68%, MS: [M+H]⁺=592).

### Synthesis Example 1-45

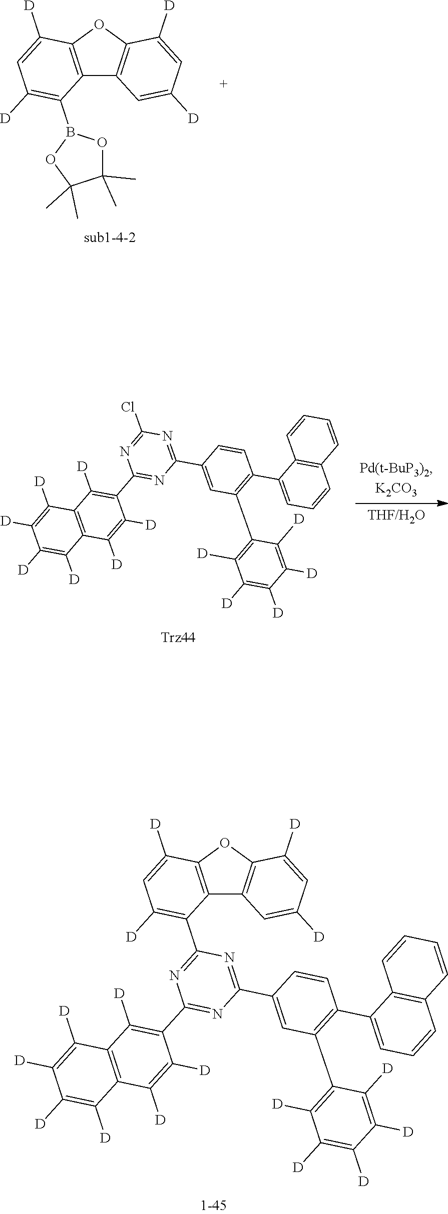 [SMILES: [2H]c1cc([2H])c2oc3c([2H])cc([2H])c(B4OC(C)(C)C(C)(C)O4)c3c2c1 ; [2H]c1c([2H])c([2H])c(-c2cc(-c3nc(Cl)nc(-c4c([2H])c([2H])c5c([2H])c([2H])c([2H])c([2H])c5c4[2H])n3)ccc2-c2cccc3ccccc23)c([2H])c1[2H] ; [2H]c1cc([2H])c2oc3c([2H])cc([2H])c(-c4nc(-c5ccc(-c6cccc7ccccc67)c(-c6c([2H])c([2H])c([2H])c([2H])c6[2H])c5)nc(-c5c([2H])c([2H])c6c([2H])c([2H])c([2H])c([2H])c6c5[2H])n4)c3c2c1]

sub1-4-2 (15 g, 50.3 mmol) and Trz44 (28.1 g, 52.8 mmol) were added to 300 mL of THF, and the mixture was stirred and refluxed. Then, potassium carbonate (20.9 g, 150.9 mmol) was dissolved in 63 ml of water, added thereto, sufficiently stirred, and then bis(tri-tert-butylphosphine)palladium(0) (0.3 g, 0.5 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 24.2 g of Compound 1-45. (Yield: 72%, MS: [M+H]⁺=668).

### Synthesis Example 1-46

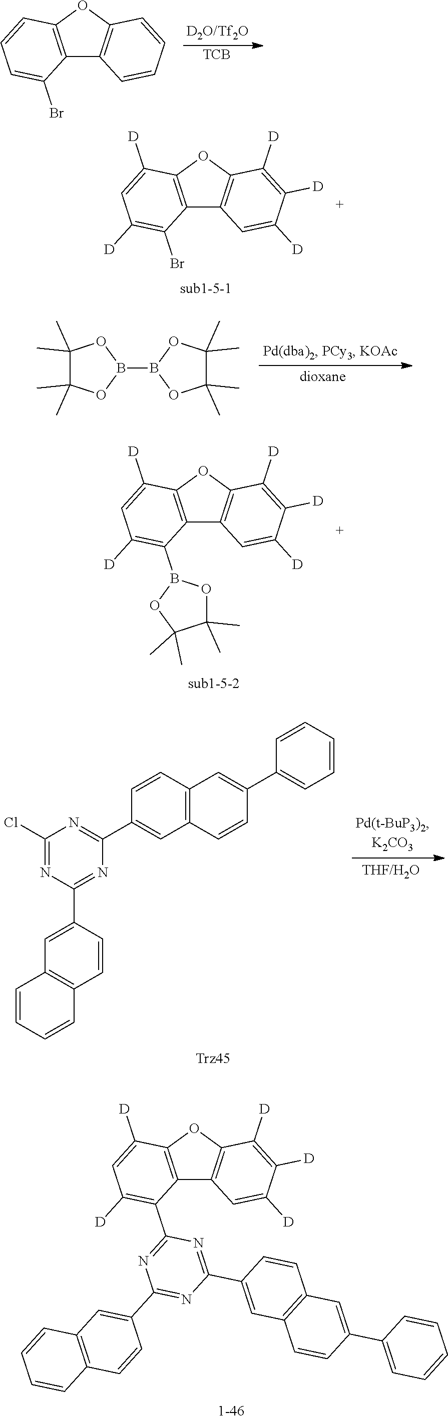

Trifluoromethanesulfonic anhydride (119.9 g, 424.9 mmol) and deuterium oxide (42.6 g, 2124.7 mmol) were added at 0° C., and the mixture was stirred for 5 hours to prepare a solution. 1-Bromodibenzo[b,d]furan (15 g, 60.7 mmol) was added to 120 mL of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating up to 140° C. and then keeping that temperature. After the reaction for 24 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.9 g of sub1-5-1. (Yield: 39%, MS: [M+H]⁺=252)

sub1-5-1 (15 g, 59.5 mmol) and bis(pinacolato)diboron (16.6 g, 65.4 mmol) were added to 300 ml of 1,4-dioxane, and the mixture was stirred under reflux. Then, potassium acetate (8.8 g, 89.2 mmol) was added thereto, sufficiently stirred, and then bis(dibenzylideneacetone)palladium(0) (1 g, 1.8 mmol) and tricyclohexylphosphine (1 g, 3.6 mmol) were added. After the reaction for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by a silica gel column chromatography to prepare 11.2 g of sub1-5-2. (Yield: 63%, MS: [M+H]⁺=300)

sub1-5-2 (15 g, 50.1 mmol) and Trz45 (23.4 g, 52.6 mmol) were added to 300 ml of THF, and the mixture was stirred and refluxed. Then, potassium carbonate (20.8 g, 150.4 mmol) was dissolved in 62 ml of water, added thereto, sufficiently stirred, and then bis(tri-tert-butylphosphine)palladium(0) (0.3 g, 0.5 mmol) was added. After the reaction for 4 hours, the reaction mixture was cooled to room temperature, and then the organic layer and the aqueous layer were separated, and the organic layer was distilled. This was dissolved again in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 20.1 g of Compound 1-46. (Yield: 69%, MS: [M+H]⁺=581)

### Synthesis Example 1-47

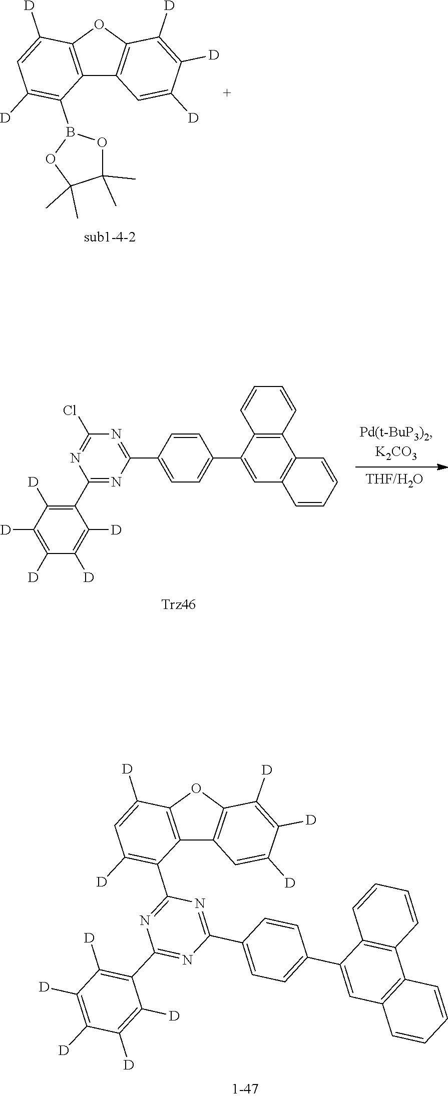 [SMILES: [2H]c1cc2c(oc3c([2H])cc([2H])c(B4OC(C)(C)C(C)(C)O4)c32)c([2H])c1[2H] ; [2H]c1c([2H])c([2H])c(-c2nc(Cl)nc(-c3ccc(-c4cc5ccccc5c5ccccc45)cc3)n2)c([2H])c1[2H] ; [2H]c1cc2c(oc3c([2H])cc([2H])c(-c4nc(-c5ccc(-c6cc7ccccc7c7ccccc67)cc5)nc(-c5c([2H])c([2H])c([2H])c([2H])c5[2H])n4)c32)c([2H])c1[2H]]

sub1-5-2 (15 g, 50.1 mmol) and Trz46 (23.6 g, 52.6 mmol) were added to 300 mL of THF, and the mixture was stirred and refluxed. Then, potassium carbonate (20.8 g, 150.4 mmol) was dissolved in 62 ml of water, added thereto, sufficiently stirred, and then bis(tri-tert-butylphosphine)palladium(0) (0.3 g, 0.5 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 20.2 g of Compound 1-47. (Yield: 69%, MS: [M+H]⁺=586).

### Synthesis Example 1-48

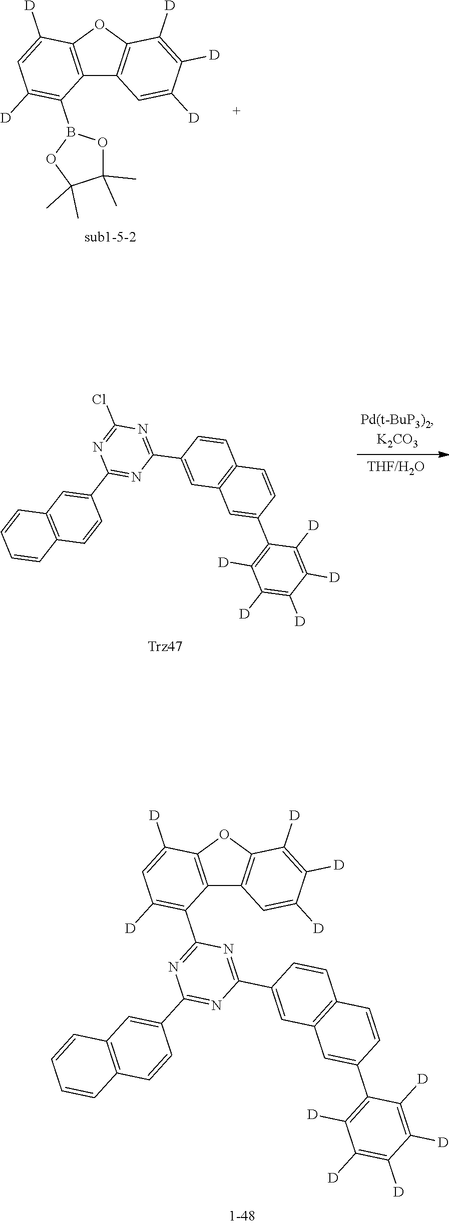 [SMILES: [2H]c1cc2c(oc3c([2H])cc([2H])c(B4OC(C)(C)C(C)(C)O4)c32)c([2H])c1[2H] ; [2H]c1c([2H])c([2H])c(-c2ccc3ccc(-c4nc(Cl)nc(-c5ccc6ccccc6c5)n4)cc3c2)c([2H])c1[2H] ; [2H]c1cc2c(oc3c([2H])cc([2H])c(-c4nc(-c5ccc6ccccc6c5)nc(-c5ccc6ccc(-c7c([2H])c([2H])c([2H])c([2H])c7[2H])cc6c5)n4)c32)c([2H])c1[2H]]

sub1-5-2 (15 g, 50.1 mmol) and Trz47 (23.6 g, 52.6 mmol) were added to 300 mL of THF, and the mixture was stirred and refluxed. Then, potassium carbonate (20.8 g, 150.4 mmol) was dissolved in 62 ml of water, added thereto, sufficiently stirred, and then bis(tri-tert-butylphosphine)palladium(0) (0.3 g, 0.5 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 21.7 g of Compound 1-48. (Yield: 74%, MS: [M+H]⁺=586).

### Synthesis Example 1-49

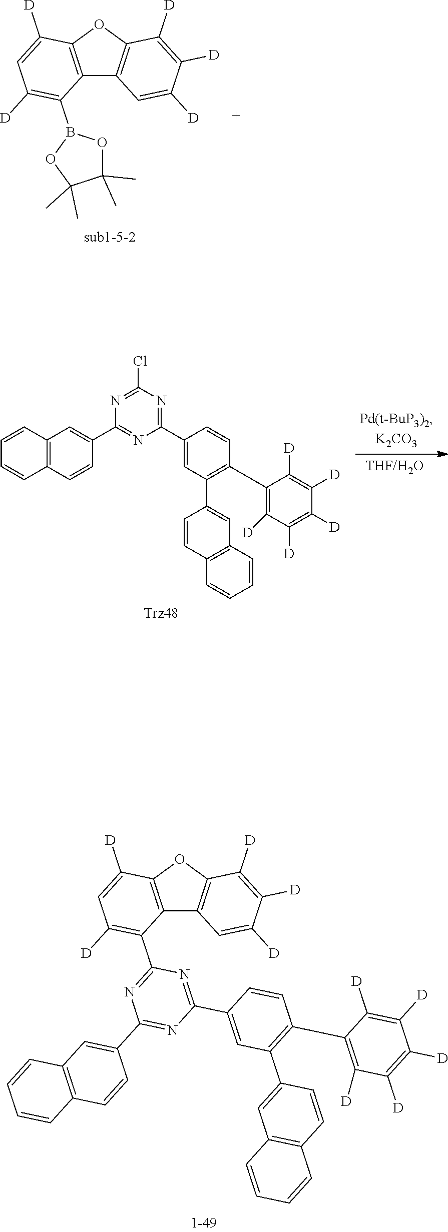 [SMILES: [2H]c1cc2c(oc3c([2H])cc([2H])c(B4OC(C)(C)C(C)(C)O4)c32)c([2H])c1[2H] ; [2H]c1c([2H])c([2H])c(-c2ccc(-c3nc(Cl)nc(-c4ccc5ccccc5c4)n3)cc2-c2ccc3ccccc3c2)c([2H])c1[2H] ; [2H]c1cc2c(oc3c([2H])cc([2H])c(-c4nc(-c5ccc(-c6c([2H])c([2H])c([2H])c([2H])c6[2H])c(-c6ccc7ccccc7c6)c5)nc(-c5ccc6ccccc6c5)n4)c32)c([2H])c1[2H]]

sub1-5-2 (15 g, 50.1 mmol) and Trz48 (27.6 g, 52.6 mmol) were added to 300 mL of THF, and the mixture was stirred and refluxed. Then, potassium carbonate (20.8 g, 150.4 mmol) was dissolved in 62 ml of water, added thereto, sufficiently stirred, and then bis(tri-tert-butylphosphine)palladium(0) (0.3 g, 0.5 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 22.5 g of Compound 1-49. (Yield: 68%, MS: [M+H]⁺=662).

### Synthesis Example 1-50

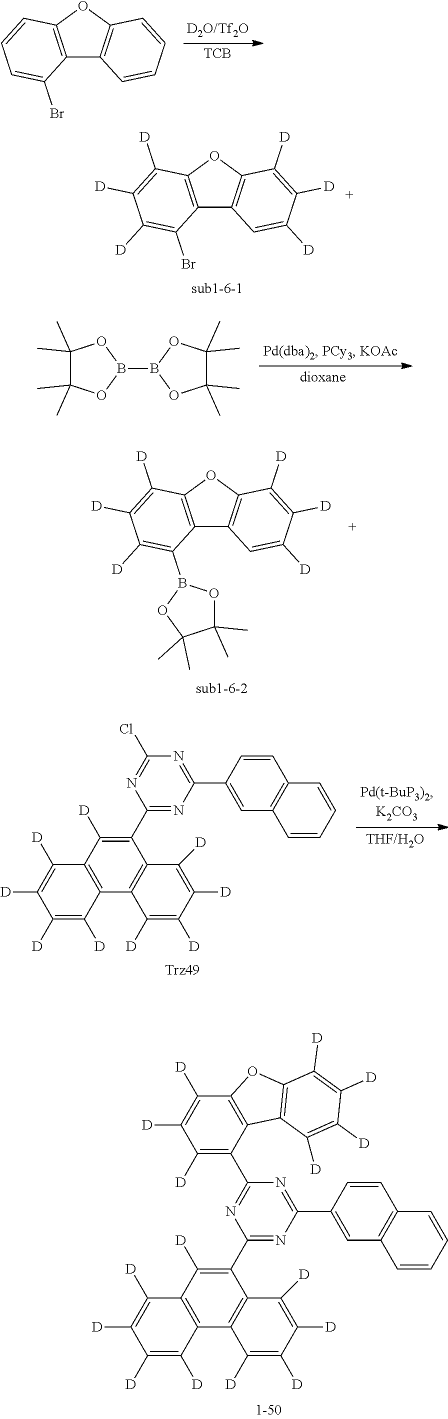

Trifluoromethanesulfonic anhydride (167.8 g, 594.9 mmol) and deuterium oxide (59.6 g, 2974.6 mmol) were added at 0° C., and the mixture was stirred for 5 hours to prepare a solution. 1-Bromodibenzo[b,d]furan (15 g, 60.7 mmol) was added to 120 mL of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating up to 140° C. and then keeping that temperature. After the reaction for 36 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.1 g of sub1-6-1. (Yield: 40%, MS: [M+H]⁺=254)

sub1-6-1 (15 g, 59 mmol) and bis(pinacolato)diboron (16.5 g, 64.9 mmol) were added to 300 ml of 1,4-dioxane, and the mixture was stirred under reflux. Then, potassium acetate (8.7 g, 88.5 mmol) was added thereto, sufficiently stirred, and then bis(dibenzylideneacetone)palladium(0) (1 g, 1.8 mmol) and tricyclohexylphosphine (1 g, 3.5 mmol) were added. After the reaction for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by a silica gel column chromatography to prepare 11.6 g of sub1-6-2. (Yield: 65%, MS: [M+H]⁺=302)

sub1-6-2 (15 g, 49.8 mmol) and Trz49 (22.3 g, 52.3 mmol) were added to 300 ml of THF, and the mixture was stirred and refluxed. Then, potassium carbonate (20.6 g, 149.4 mmol) was dissolved in 62 ml of water, added thereto, sufficiently stirred, and then bis(tri-tert-butylphosphine)palladium(0) (0.3 g, 0.5 mmol) was added. After the reaction for 5 hours, the reaction mixture was cooled to room temperature, and then the organic layer and the aqueous layer were separated, and the organic layer was distilled. This was dissolved again in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 20.3 g of Compound 1-50. (Yield: 72%, MS: [M+H]⁺=566)

### Synthesis Example 1-51

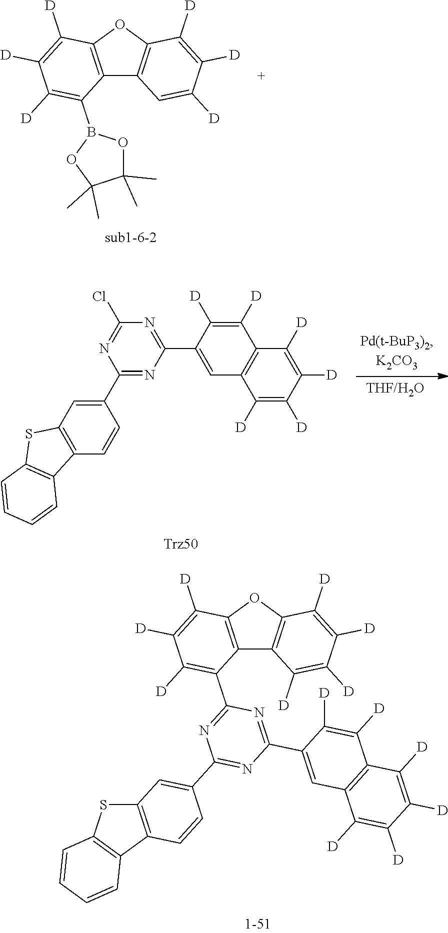 [SMILES: [2H]c1cc2c(oc3c([2H])c([2H])c([2H])c(B4OC(C)(C)C(C)(C)O4)c32)c([2H])c1[2H] ; [2H]c1c([2H])c([2H])c2c([2H])c([2H])c(-c3nc(Cl)nc(-c4ccc5c(c4)sc4ccccc45)n3)cc2c1[2H] ; [2H]c1c([2H])c([2H])c2c([2H])c([2H])c(-c3nc(-c4ccc5c(c4)sc4ccccc45)nc(-c4c([2H])c([2H])c([2H])c5oc6c([2H])c([2H])c([2H])c([2H])c6c45)n3)cc2c1[2H]]

sub1-6-2 (15 g, 49.8 mmol) and Trz50 (22.5 g, 52.3 mmol) were added to 300 mL of THF, and the mixture was stirred and refluxed. Then, potassium carbonate (20.6 g, 149.4 mmol) was dissolved in 62 ml of water, added thereto, sufficiently stirred, and then bis(tri-tert-butylphosphine)palladium(0) (0.3 g, 0.5 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 20.4 g of Compound 1-51. (Yield: 72%, MS: [M+H]⁺=569).

### Synthesis Example 1-52

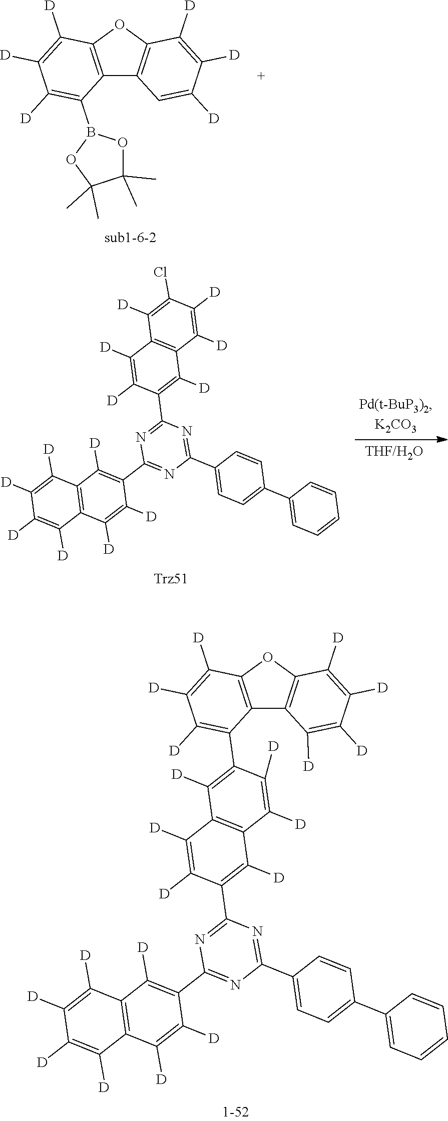 [SMILES: [2H]c1cc2c(oc3c([2H])c([2H])c([2H])c(B4OC(C)(C)C(C)(C)O4)c32)c([2H])c1[2H] ; [2H]c1c([2H])c([2H])c2c([2H])c(-c3nc(-c4ccc(-c5ccccc5)cc4)nc(-c4c([2H])c([2H])c5c([2H])c(Cl)c([2H])c([2H])c5c4[2H])n3)c([2H])c([2H])c2c1[2H] ; [2H]c1c([2H])c([2H])c2c(oc3c([2H])c([2H])c([2H])c(-c4c([2H])c([2H])c5c([2H])c(-c6nc(-c7ccc(-c8ccccc8)cc7)nc(-c7c([2H])c([2H])c8c([2H])c([2H])c([2H])c([2H])c8c7[2H])n6)c([2H])c([2H])c5c4[2H])c32)c1[2H]]

sub1-6-2 (15 g, 49.8 mmol) and Trz51 (27.9 g, 52.3 mmol) were added to 300 mL of THF, and the mixture was stirred and refluxed. Then, potassium carbonate (20.6 g, 149.4 mmol) was dissolved in 62 ml of water, added thereto, sufficiently stirred, and then bis(tri-tert-butylphosphine)palladium(0) (0.3 g, 0.5 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 24.7 g of Compound 1-52. (Yield: 74%, MS: [M+H]⁺=672).

### Synthesis Example 1-53

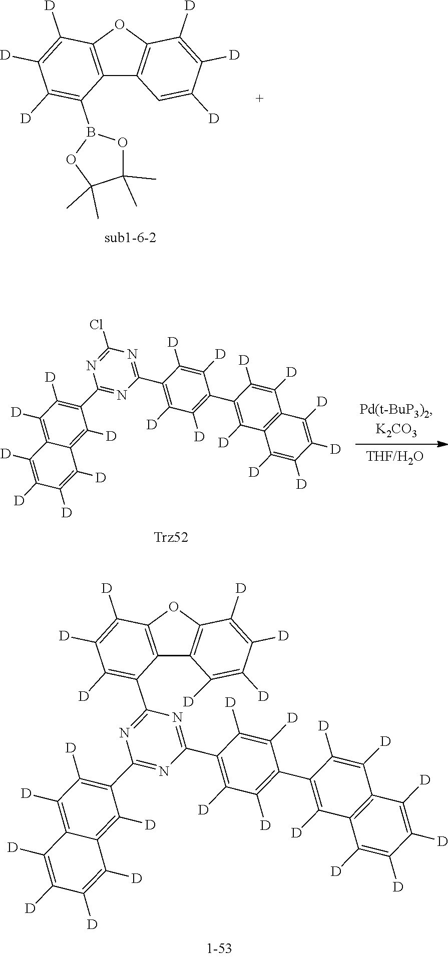 [SMILES: [2H]c1cc2c(oc3c([2H])c([2H])c([2H])c(B4OC(C)(C)C(C)(C)O4)c32)c([2H])c1[2H] ; [2H]c1c([2H])c(-c2c([2H])c([2H])c3c([2H])c([2H])c([2H])c([2H])c3c2[2H])c([2H])c([2H])c1-c1nc(Cl)nc(-c2c([2H])c([2H])c3c([2H])c([2H])c([2H])c([2H])c3c2[2H])n1 ; [2H]c1c([2H])c(-c2c([2H])c([2H])c3c([2H])c([2H])c([2H])c([2H])c3c2[2H])c([2H])c([2H])c1-c1nc(-c2c([2H])c([2H])c3c([2H])c([2H])c([2H])c([2H])c3c2[2H])nc(-c2c([2H])c([2H])c([2H])c3oc4c([2H])c([2H])c([2H])c([2H])c4c23)n1]

sub1-6-2 (15 g, 49.8 mmol) and Trz52 (24.2 g, 52.3 mmol) were added to 300 mL of THF, and the mixture was stirred and refluxed. Then, potassium carbonate (20.6 g, 149.4 mmol) was dissolved in 62 ml of water, added thereto, sufficiently stirred, and then bis(tri-tert-butylphosphine)palladium(0) (0.3 g, 0.5 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 22.4 g of Compound 1-53. (Yield: 75%, MS: [M+H]⁺=601).

### Synthesis Example 1-54

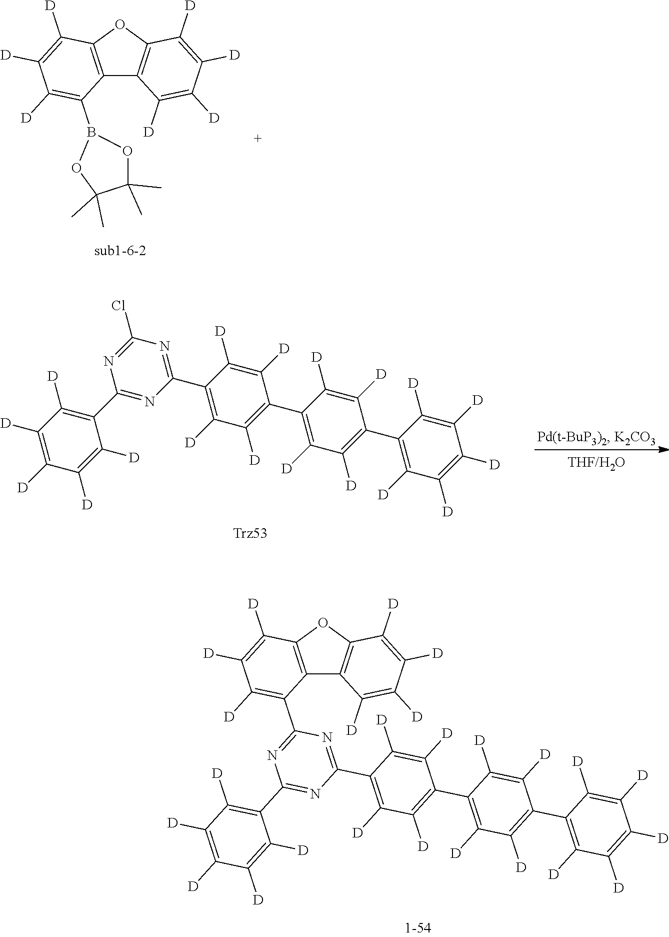 [SMILES: [2H]c1c([2H])c([2H])c2c(oc3c([2H])c([2H])c([2H])c(B4OC(C)(C)C(C)(C)O4)c32)c1[2H] ; [2H]c1c([2H])c([2H])c(-c2nc(Cl)nc(-c3c([2H])c([2H])c(-c4c([2H])c([2H])c(-c5c([2H])c([2H])c([2H])c([2H])c5[2H])c([2H])c4[2H])c([2H])c3[2H])n2)c([2H])c1[2H] ; [2H]c1c([2H])c([2H])c(-c2nc(-c3c([2H])c([2H])c(-c4c([2H])c([2H])c(-c5c([2H])c([2H])c([2H])c([2H])c5[2H])c([2H])c4[2H])c([2H])c3[2H])nc(-c3c([2H])c([2H])c([2H])c4oc5c([2H])c([2H])c([2H])c([2H])c5c34)n2)c([2H])c1[2H]]

sub1-6-2 (15 g, 49.8 mmol) and Trz53 (22.9 g, 52.3 mmol) were added to 300 mL of THF, and the mixture was stirred and refluxed. Then, potassium carbonate (20.6 g, 149.4 mmol) was dissolved in 62 ml of water, added thereto, sufficiently stirred, and then bis(tri-tert-butylphosphine)palladium(0) (0.3 g, 0.5 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 18.7 g of Compound 1-54. (Yield: 65%, MS: [M+H]⁺=577).

### Synthesis Example 1-55

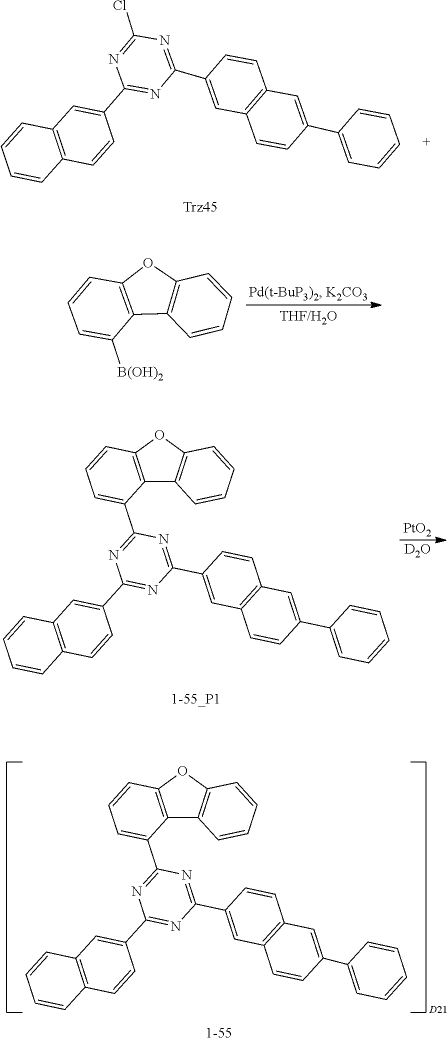

Trz45 (15 g, 33.8 mmol) and dibenzo[b,d]furan-1-ylboronic acid (7.5 g, 35.5 mmol) were added to 300 mL of THF, and the mixture was stirred and refluxed. Then, potassium carbonate (14 g, 101.4 mmol) was dissolved in 42 ml of water, added thereto, sufficiently stirred, and then bis(tri-tert-butylphosphine)palladium(0) (0.2 g, 0.3 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 12.8 g of Compound 1-55_P1. (Yield: 66%, MS: [M+H]⁺=576).

Compound 1-55_P1 (10 g, 17.4 mmol), PtO₂ (1.2 g, 5.2 mmol) and D₂O (87 ml) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 4.1 g of Compound 1-55. (Yield: 40%, MS: [M+H]⁺=598)

### Synthesis Example 1-56

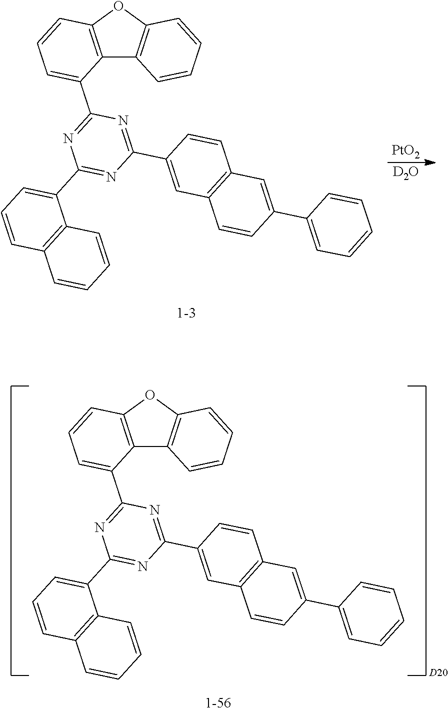

Compound 1-3 (10 g, 17.4 mmol), PtO₂ (1.2 g, 5.2 mmol), D₂O (87 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 4.4 g of Compound 1-56. At this time, the deuterium substitution rate was calculated as a percentage of the number of substituted deuterium atoms to the total number of hydrogen atoms that may exist in the chemical formula after obtaining the number of substituted deuterium atoms in the compound through MALDI-TOF MS (Matrix-Assisted Laser Desorption/Ionization Time-of-Flight Mass Spectrometer) analysis. (Yield: 43%, MS: [M+H]⁺=597)

### Synthesis Example 1-57

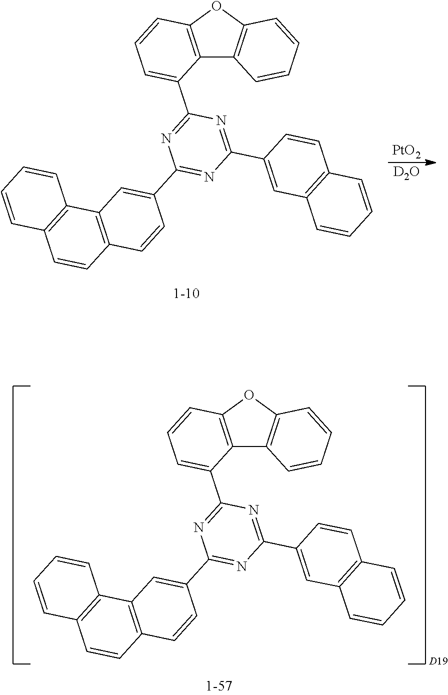

Compound 1-10 (10 g, 18.2 mmol), PtO₂ (1.2 g, 5.5 mmol) and D2O (91 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 4.1 g of Compound 1-57. At this time, the deuterium substitution rate was calculated in the same manner as in Compound 1-56. (Yield: 40%, MS: [M+H]⁺=570)

### Synthesis Example 1-58

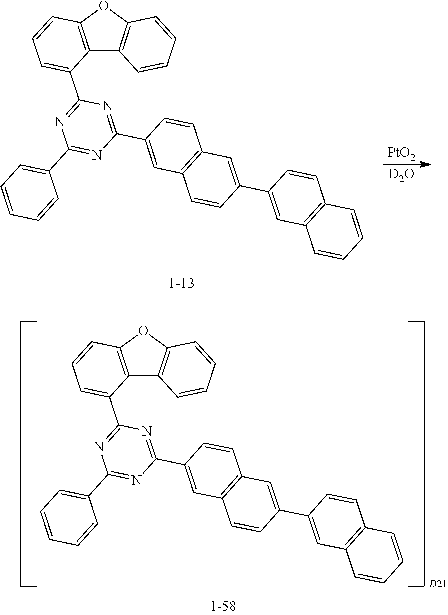

Compound 1-13 (10 g, 17.4 mmol), PtO₂ (1.2 g, 5.2 mmol), D2O (87 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 4.5 g of Compound 1-58. At this time, the deuterium substitution rate was calculated in the same manner as in Compound 1-56. (Yield: 43%, MS: [M+H]⁺=598)

### Synthesis Example 1-59

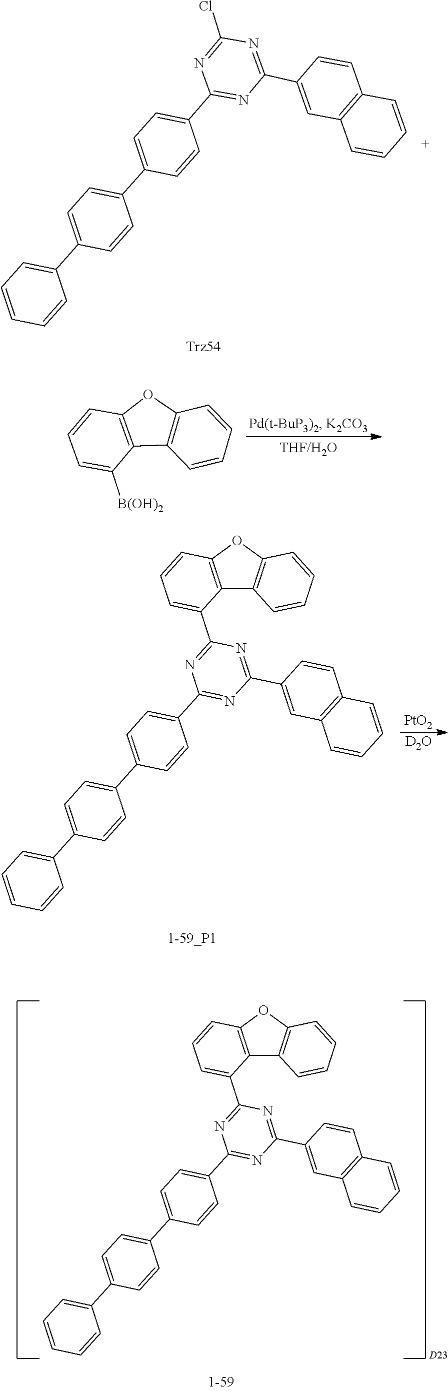

Trz54 (15 g, 31.9 mmol) and dibenzo[b,d]furan-1-ylboronic acid (7.1 g, 33.5 mmol) were added to 300 mL of THF, and the mixture was stirred and refluxed. Then, potassium carbonate (13.2 g, 95.8 mmol) was dissolved in 40 ml of water, added thereto, sufficiently stirred, and then bis(tri-tert-butylphosphine)palladium(0) (0.2 g, 0.3 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 14.2 g of Compound 1-59_P1. (Yield: 74%, MS: [M+H]⁺=602).

Compound 1-59_P1 (10 g, 16.6 mmol), PtO₂ (1.1 g, 5 mmol) and D2O (83 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 4.5 g of Compound 1-59. At this time, the deuterium substitution rate was calculated in the same manner as in Compound 1-56. (Yield: 43%, MS: [M+H]⁺=626)

### Synthesis Example 1-60

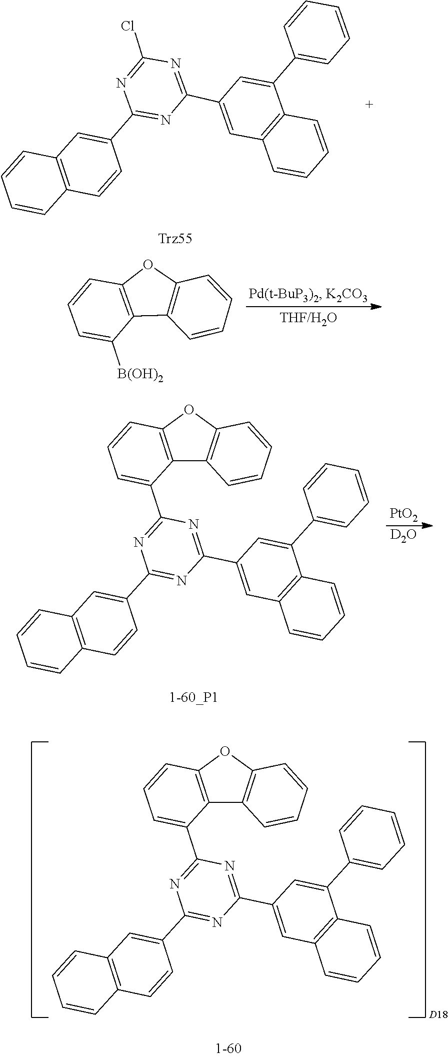

Trz55 (15 g, 33.8 mmol) and dibenzo[b,d]furan-1-ylboronic acid (7.5 g, 35.5 mmol) were added to 300 mL of THF, and the mixture was stirred and refluxed. Then, potassium carbonate (14 g, 101.4 mmol) was dissolved in 42 ml of water, added thereto, sufficiently stirred, and then bis(tri-tert-butylphosphine)palladium(0) (0.2 g, 0.3 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 13.2 g of Compound 1-60_P1. (Yield: 68%, MS: [M+H]⁺=576).

Compound 1-60_P1 (10 g, 17.4 mmol), PtO₂ (1.2 g, 5.2 mmol) and D2O (87 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 5.2 g of Compound 1-60. At this time, the deuterium substitution rate was calculated in the same manner as in Compound 1-56. (Yield: 50%, MS: [M+H]⁺=595)

### Synthesis Example 1-61

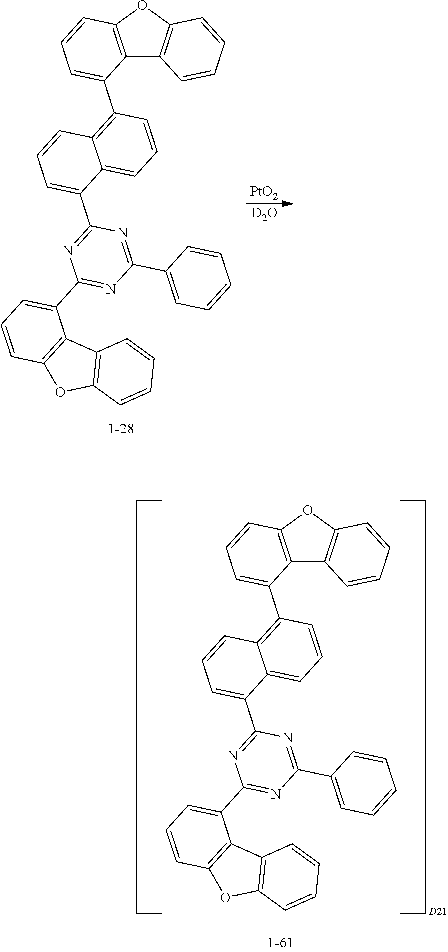

Compound 1-28 (10 g, 16.2 mmol), PtO₂ (1.1 g, 4.9 mmol) and D2O (81 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 5 g of Compound 1-61. At this time, the deuterium substitution rate was calculated in the same manner as in Compound 1-56. (Yield: 48%, MS: [M+H]⁺=638)

### [Preparation of Compound Represented by Chemical Formula 2]

### Synthesis Example 2-1

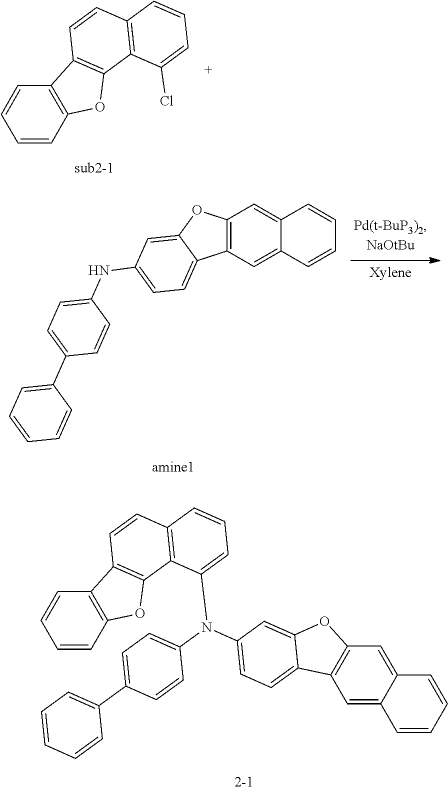 [SMILES: Clc1cccc2ccc3c4ccccc4oc3c12 ; c1ccc(-c2ccc(Nc3ccc4c(c3)oc3cc5ccccc5cc34)cc2)cc1 ; c1ccc(-c2ccc(N(c3ccc4c(c3)oc3cc5ccccc5cc34)c3cccc4ccc5c6ccccc6oc5c34)cc2)cc1]

sub2-1 (15 g, 59.4 mmol), amine1 (24 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 18.2 g of Compound 2-1. (Yield: 51%, MS: [M+H]⁺=602)

### Synthesis Example 2-2

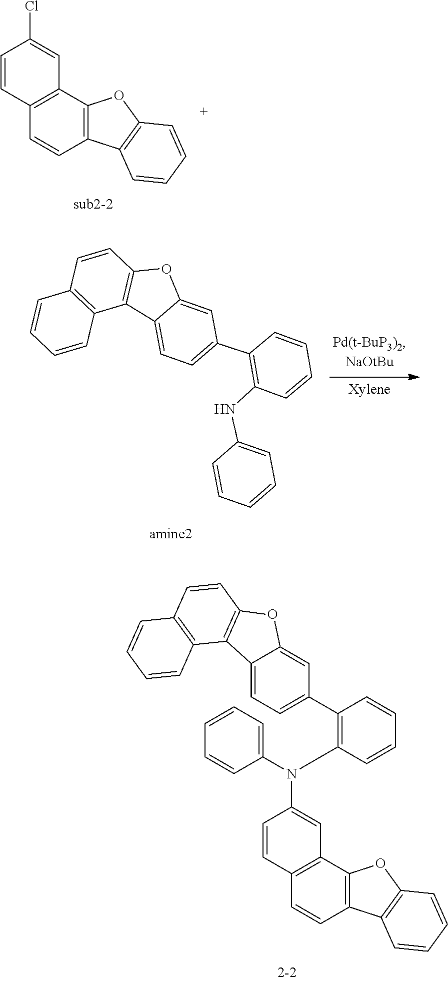 [SMILES: Clc1ccc2ccc3c4ccccc4oc3c2c1 ; c1ccc(Nc2ccccc2-c2ccc3c(c2)oc2ccc4ccccc4c23)cc1 ; c1ccc(N(c2ccc3ccc4c5ccccc5oc4c3c2)c2ccccc2-c2ccc3c(c2)oc2ccc4ccccc4c23)cc1]

sub2-2 (15 g, 59.4 mmol), amine2 (24 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 19.3 g of Compound 2-2. (Yield: 54%, MS: [M+H]⁺=602)

### Synthesis Example 2-3

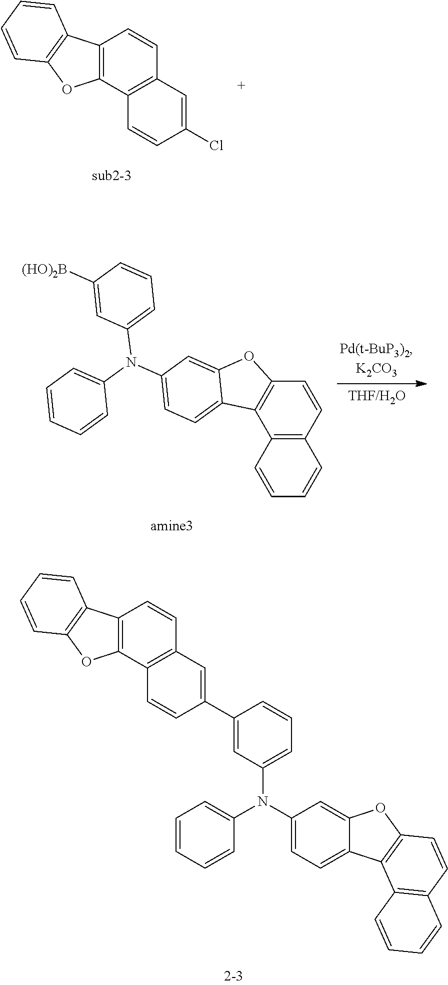 [SMILES: Clc1ccc2c(ccc3c4ccccc4oc23)c1 ; OB(O)c1cccc(N(c2ccccc2)c2ccc3c(c2)oc2ccc4ccccc4c23)c1 ; c1ccc(N(c2cccc(-c3ccc4c(ccc5c6ccccc6oc45)c3)c2)c2ccc3c(c2)oc2ccc4ccccc4c23)cc1]

sub2-3 (15 g, 59.4 mmol) and amine3 (26.8 g, 62.3 mmol) were added to 300 ml of THF, and the mixture was stirred and refluxed. Then, potassium carbonate (24.6 g, 178.1 mmol) was dissolved in 100 ml of water, added thereto, sufficiently stirred, and then bis(tri-tert-butylphosphine)palladium(0) (0.3 g, 0.6 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 22.1 g of Compound 2-3. (Yield: 62%, MS: [M+H]⁺=602).

### Synthesis Example 2-4

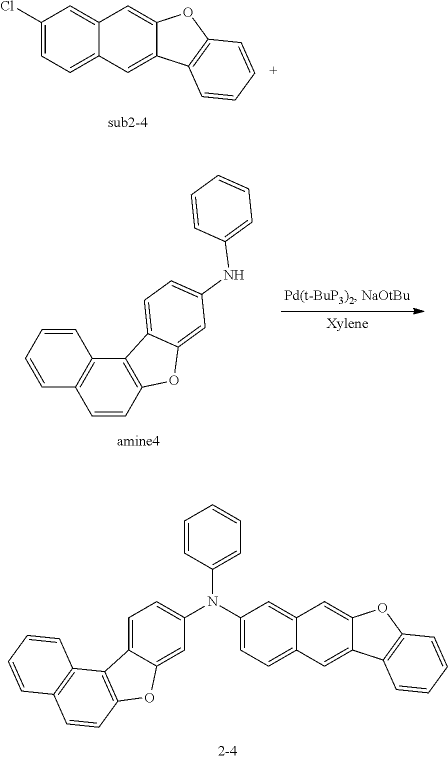 [SMILES: Clc1ccc2cc3c(cc2c1)oc1ccccc13 ; c1ccc(Nc2ccc3c(c2)oc2ccc4ccccc4c23)cc1 ; c1ccc(N(c2ccc3cc4c(cc3c2)oc2ccccc24)c2ccc3c(c2)oc2ccc4ccccc4c23)cc1]

sub2-4 (15 g, 59.4 mmol), amine4 (19.3 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 16.5 g of Compound 2-4. (Yield: 53%, MS: [M+H]⁺=526).

### Synthesis Example 2-5

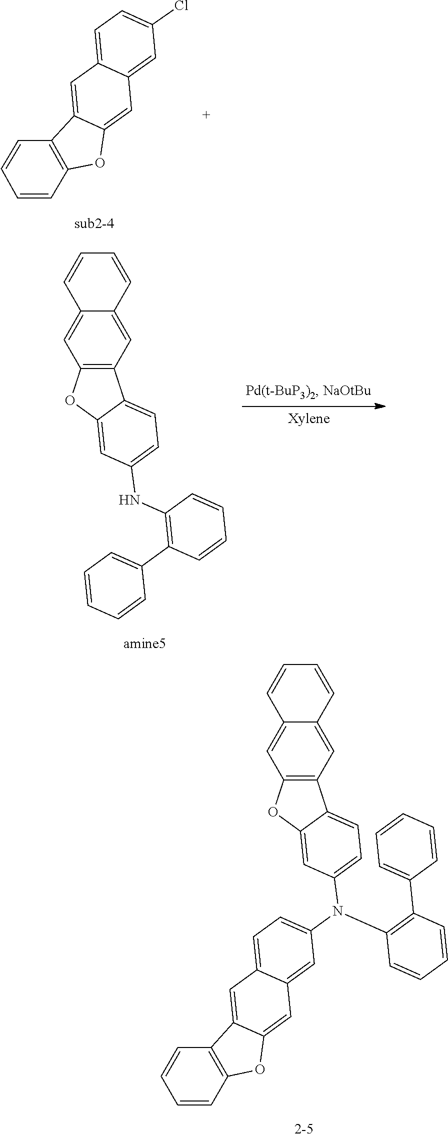 [SMILES: Clc1ccc2cc3c(cc2c1)oc1ccccc13 ; c1ccc(-c2ccccc2Nc2ccc3c(c2)oc2cc4ccccc4cc23)cc1 ; c1ccc(-c2ccccc2N(c2ccc3cc4c(cc3c2)oc2ccccc24)c2ccc3c(c2)oc2cc4ccccc4cc23)cc1]

sub2-4 (15 g, 59.4 mmol), amine5 (24 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 22.1 g of Compound 2-5. (Yield: 62%, MS: [M+H]⁺=602).

### Synthesis Example 2-6

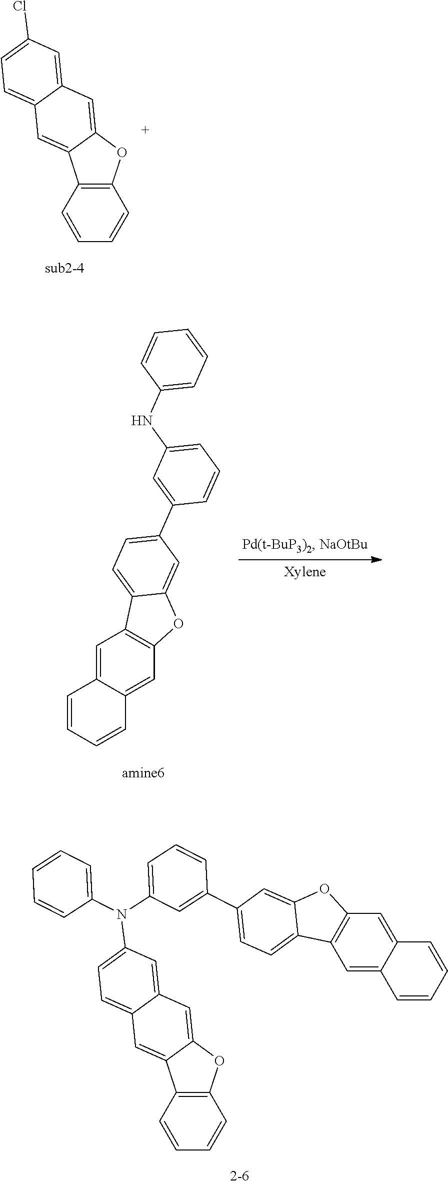 [SMILES: Clc1ccc2cc3c(cc2c1)oc1ccccc13 ; c1ccc(Nc2cccc(-c3ccc4c(c3)oc3cc5ccccc5cc34)c2)cc1 ; c1ccc(N(c2cccc(-c3ccc4c(c3)oc3cc5ccccc5cc34)c2)c2ccc3cc4c(cc3c2)oc2ccccc24)cc1]

sub2-4 (15 g, 59.4 mmol), amine6 (24 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 20.7 g of Compound 2-6. (Yield: 58%, MS: [M+H]⁺=602).

### Synthesis Example 2-7

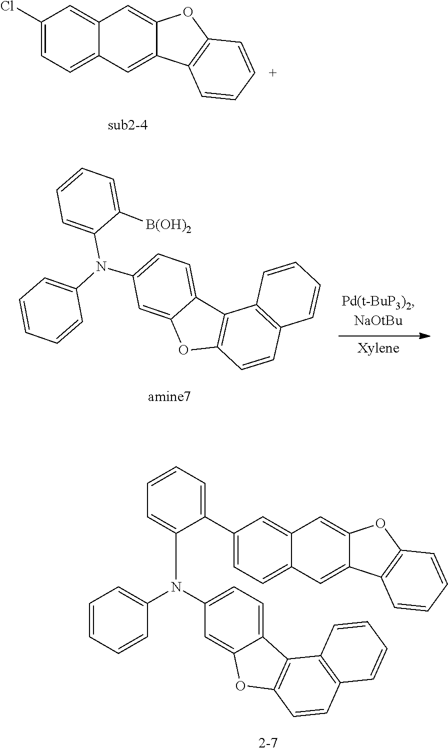 [SMILES: Clc1ccc2cc3c(cc2c1)oc1ccccc13 ; OB(O)c1ccccc1N(c1ccccc1)c1ccc2c(c1)oc1ccc3ccccc3c12 ; c1ccc(N(c2ccc3c(c2)oc2ccc4ccccc4c23)c2ccccc2-c2ccc3cc4c(cc3c2)oc2ccccc24)cc1]

sub2-4 (15 g, 59.4 mmol) and amine7 (26.8 g, 62.3 mmol) were added to 300 ml of THF, and the mixture was stirred and refluxed. Then, potassium carbonate (24.6 g, 178.1 mmol) was dissolved in 100 mL of water, added thereto, sufficiently stirred, and then bis(tri-tert-butylphosphine)palladium(0) (0.3 g, 0.6 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 22.5 g of Compound 2-7. (Yield: 63%, MS: [M+H]⁺=602).

### Synthesis Example 2-8

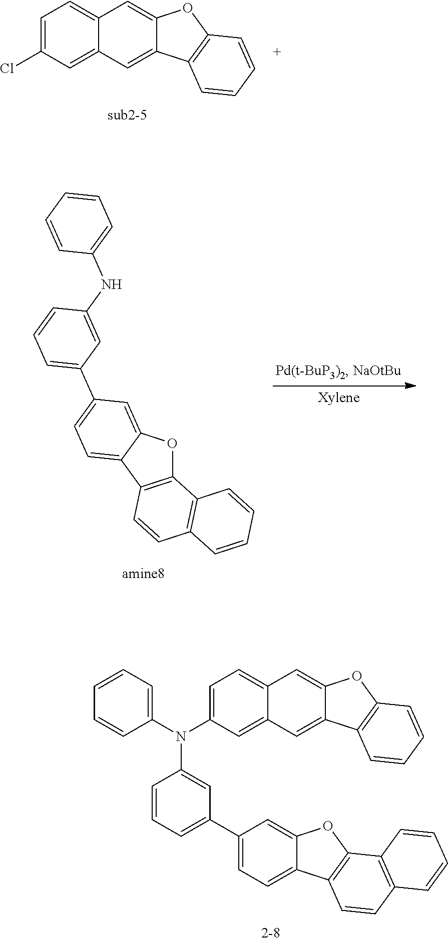 [SMILES: Clc1ccc2cc3oc4ccccc4c3cc2c1 ; c1ccc(Nc2cccc(-c3ccc4c(c3)oc3c5ccccc5ccc43)c2)cc1 ; c1ccc(N(c2cccc(-c3ccc4c(c3)oc3c5ccccc5ccc43)c2)c2ccc3cc4oc5ccccc5c4cc3c2)cc1]

sub2-5 (15 g, 59.4 mmol), amine8 (24 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 18.2 g of Compound 2-8. (Yield: 51%, MS: [M+H]⁺=602).

### Synthesis Example 2-9

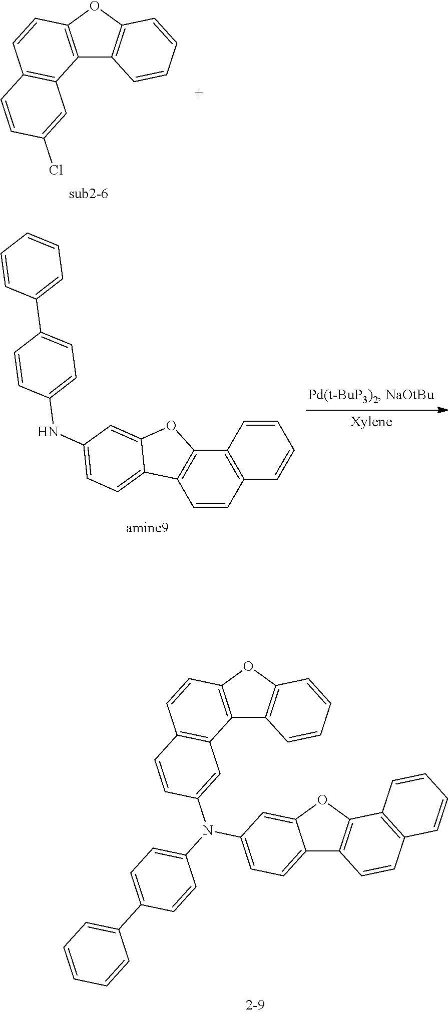 [SMILES: Clc1ccc2ccc3oc4ccccc4c3c2c1 ; c1ccc(-c2ccc(Nc3ccc4c(c3)oc3c5ccccc5ccc43)cc2)cc1 ; c1ccc(-c2ccc(N(c3ccc4c(c3)oc3c5ccccc5ccc43)c3ccc4ccc5oc6ccccc6c5c4c3)cc2)cc1]

sub2-6 (15 g, 59.4 mmol), amine9 (24 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 20.7 g of Compound 2-9. (Yield: 58%, MS: [M+H]⁺=602).

### Synthesis Example 2-10

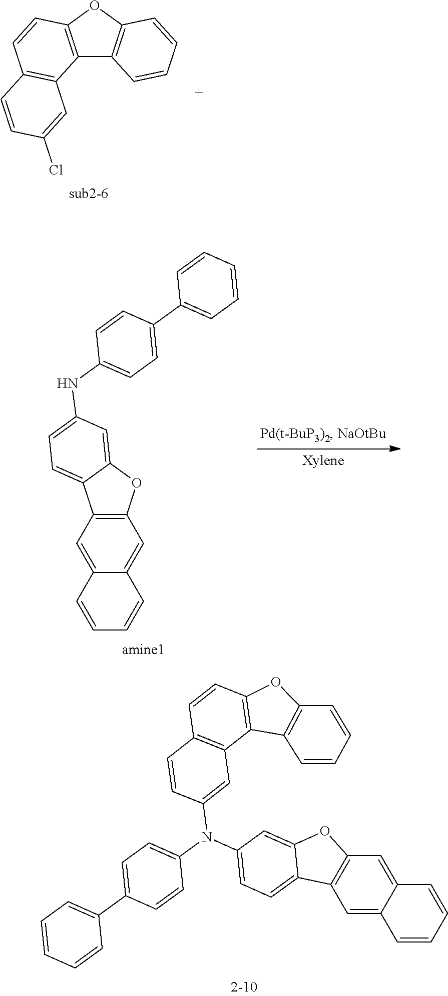 [SMILES: Clc1ccc2ccc3oc4ccccc4c3c2c1 ; c1ccc(-c2ccc(Nc3ccc4c(c3)oc3cc5ccccc5cc34)cc2)cc1 ; c1ccc(-c2ccc(N(c3ccc4c(c3)oc3cc5ccccc5cc34)c3ccc4ccc5oc6ccccc6c5c4c3)cc2)cc1]

sub2-6 (15 g, 59.4 mmol), amine1 (24 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 18.2 g of Compound 2-10. (Yield: 51%, MS: [M+H]⁺=602).

### Synthesis Example 2-11

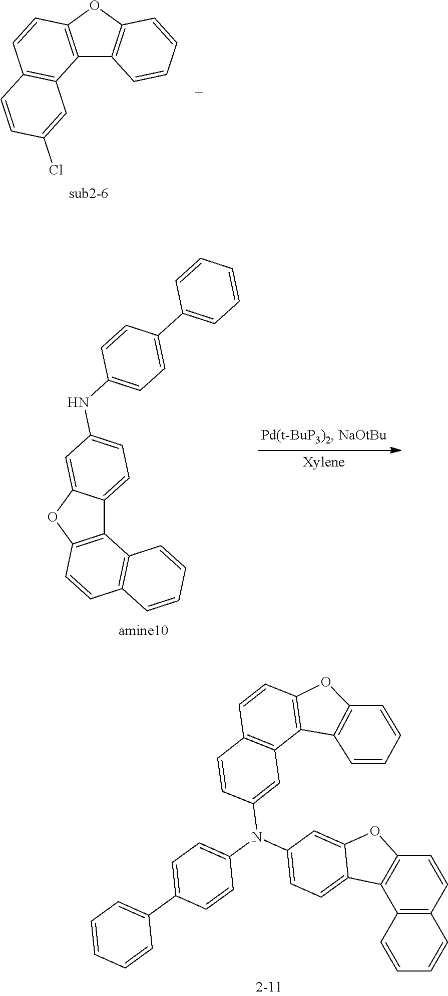 [SMILES: Clc1ccc2ccc3oc4ccccc4c3c2c1 ; c1ccc(-c2ccc(Nc3ccc4c(c3)oc3ccc5ccccc5c34)cc2)cc1 ; c1ccc(-c2ccc(N(c3ccc4c(c3)oc3ccc5ccccc5c34)c3ccc4ccc5oc6ccccc6c5c4c3)cc2)cc1]

sub2-6 (15 g, 59.4 mmol), amine10 (24 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 19.6 g of Compound 2-11. (Yield: 55%, MS: [M+H]⁺=602).

### Synthesis Example 2-12

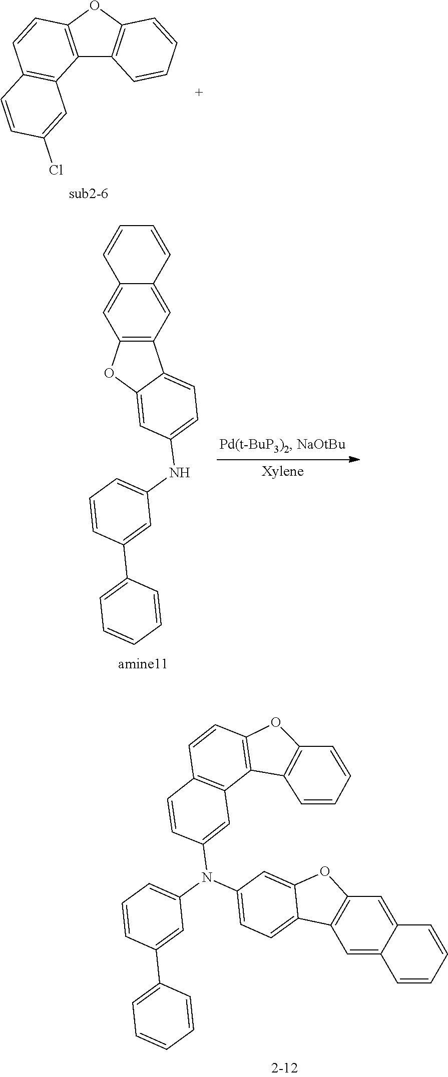 [SMILES: Clc1ccc2ccc3oc4ccccc4c3c2c1 ; c1ccc(-c2cccc(Nc3ccc4c(c3)oc3cc5ccccc5cc34)c2)cc1 ; c1ccc(-c2cccc(N(c3ccc4c(c3)oc3cc5ccccc5cc34)c3ccc4ccc5oc6ccccc6c5c4c3)c2)cc1]

sub2-6 (15 g, 59.4 mmol), amine11 (24 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 19.6 g of Compound 2-12. (Yield: 55%, MS: [M+H]⁺=602).

### Synthesis Example 2-13

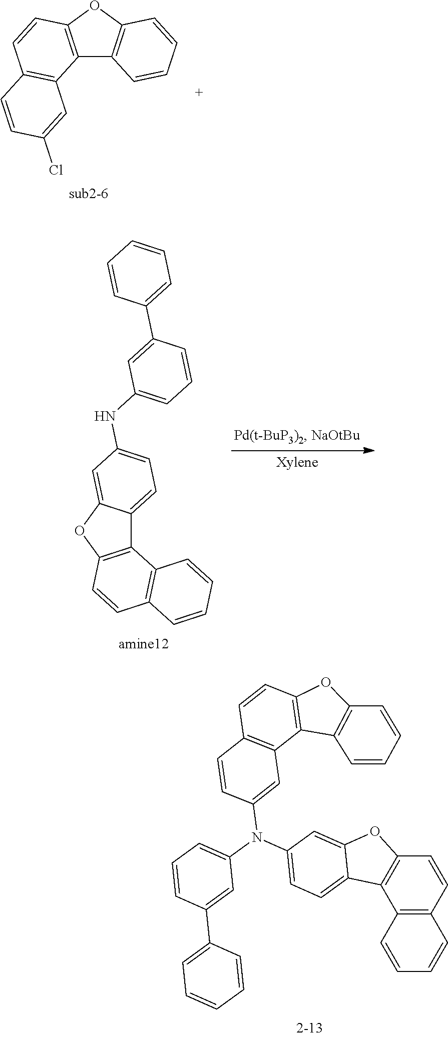 [SMILES: Clc1ccc2ccc3oc4ccccc4c3c2c1 ; c1ccc(-c2cccc(Nc3ccc4c(c3)oc3ccc5ccccc5c34)c2)cc1 ; c1ccc(-c2cccc(N(c3ccc4c(c3)oc3ccc5ccccc5c34)c3ccc4ccc5oc6ccccc6c5c4c3)c2)cc1]

sub2-6 (15 g, 59.4 mmol), amine12 (24 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 23.2 g of Compound 2-13. (Yield: 65%, MS: [M+H]⁺=602).

### Synthesis Example 2-14

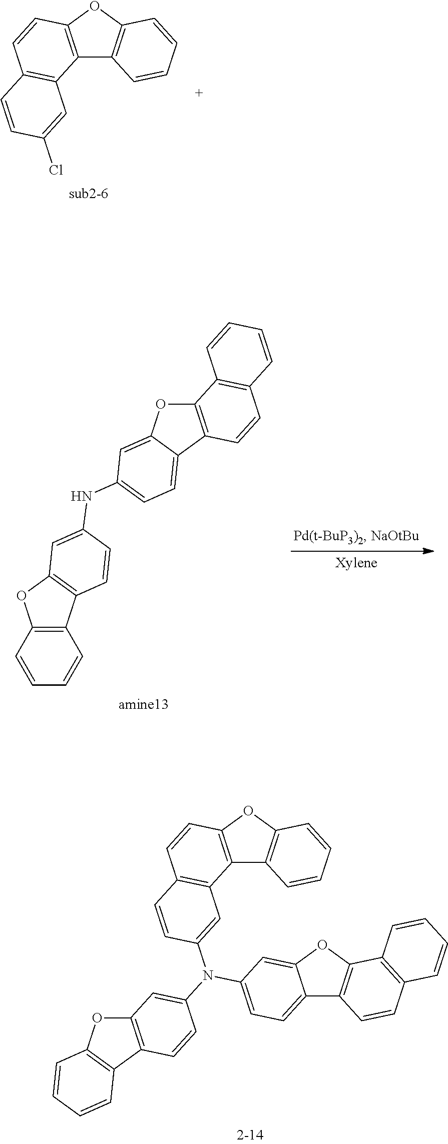 [SMILES: Clc1ccc2ccc3oc4ccccc4c3c2c1 ; c1ccc2c(c1)ccc1c3ccc(Nc4ccc5c(c4)oc4ccccc45)cc3oc21 ; c1ccc2c(c1)ccc1c3ccc(N(c4ccc5c(c4)oc4ccccc45)c4ccc5ccc6oc7ccccc7c6c5c4)cc3oc21]

sub2-6 (15 g, 59.4 mmol), amine13 (24.9 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 24.8 g of Compound 2-14. (Yield: 68%, MS: [M+H]⁺=616).

### Synthesis Example 2-15

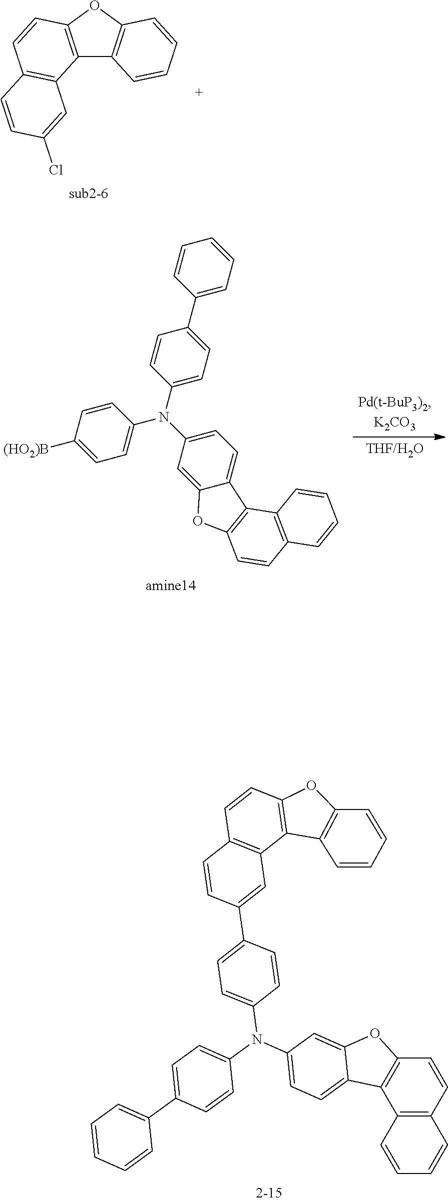

sub2-6 (15 g, 59.4 mmol) and amine14 (31.5 g, 62.3 mmol) were added to 300 ml of THF, and the mixture was stirred and refluxed. Then, potassium carbonate (24.6 g, 178.1 mmol) was dissolved in 100 mL of water, added thereto, sufficiently stirred, and then bis(tri-tert-butylphosphine)palladium(0) (0.3 g, 0.6 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 31 g of Compound 2-15. (Yield: 77%, MS: [M+H]⁺=678).

### Synthesis Example 2-16

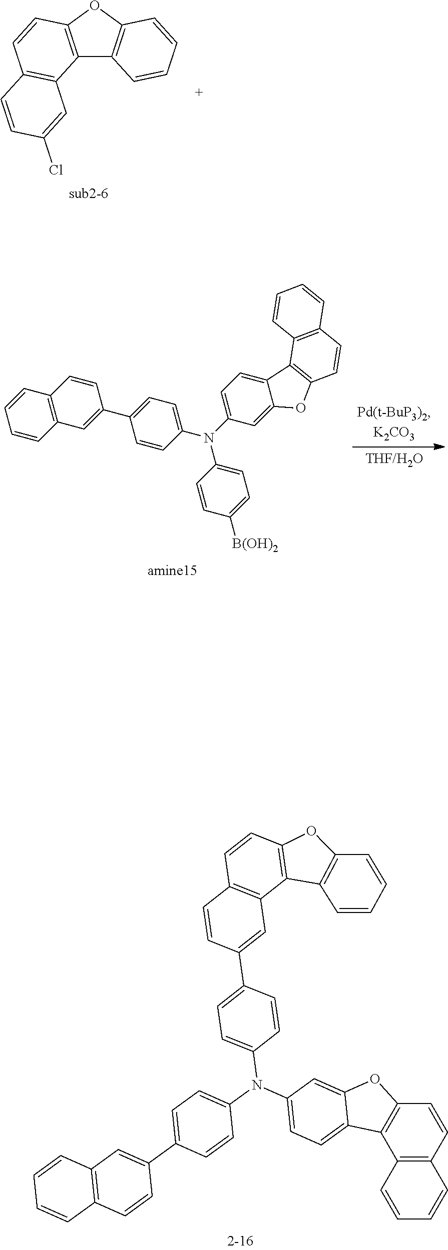 [SMILES: Clc1ccc2ccc3oc4ccccc4c3c2c1 ; OB(O)c1ccc(N(c2ccc(-c3ccc4ccccc4c3)cc2)c2ccc3c(c2)oc2ccc4ccccc4c23)cc1 ; c1ccc2cc(-c3ccc(N(c4ccc(-c5ccc6ccc7oc8ccccc8c7c6c5)cc4)c4ccc5c(c4)oc4ccc6ccccc6c45)cc3)ccc2c1]

sub2-6 (15 g, 59.4 mmol) and amine15 (34.6 g, 62.3 mmol) were added to 300 ml of THF, and the mixture was stirred and refluxed. Then, potassium carbonate (24.6 g, 178.1 mmol) was dissolved in 100 mL of water, added thereto, sufficiently stirred, and then bis(tri-tert-butylphosphine)palladium(0) (0.3 g, 0.6 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 32.4 g of Compound 2-16. (Yield: 75%, MS: [M+H]⁺=728).

### Synthesis Example 2-17

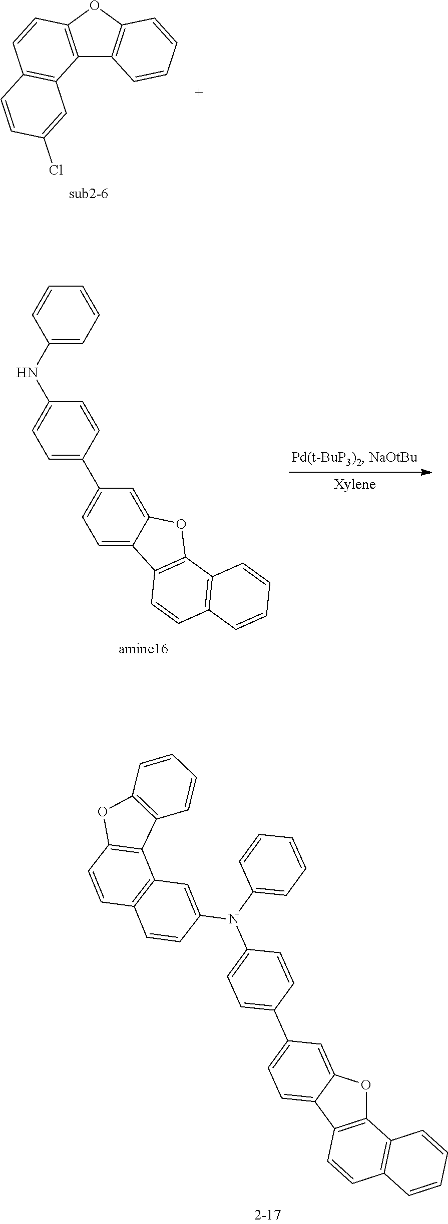 [SMILES: Clc1ccc2ccc3oc4ccccc4c3c2c1 ; c1ccc(Nc2ccc(-c3ccc4c(c3)oc3c5ccccc5ccc43)cc2)cc1 ; c1ccc(N(c2ccc(-c3ccc4c(c3)oc3c5ccccc5ccc43)cc2)c2ccc3ccc4oc5ccccc5c4c3c2)cc1]

sub2-6 (15 g, 59.4 mmol), amine16 (24 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 21.8 g of Compound 2-17. (Yield: 61%, MS: [M+H]⁺=602).

### Synthesis Example 2-18

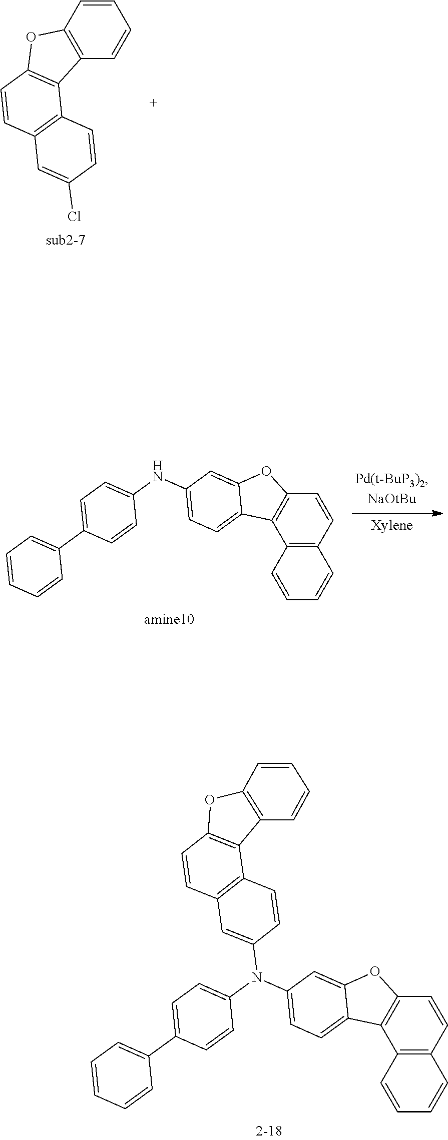 [SMILES: Clc1ccc2c(ccc3oc4ccccc4c32)c1 ; c1ccc(-c2ccc(Nc3ccc4c(c3)oc3ccc5ccccc5c34)cc2)cc1 ; c1ccc(-c2ccc(N(c3ccc4c(ccc5oc6ccccc6c54)c3)c3ccc4c(c3)oc3ccc5ccccc5c34)cc2)cc1]

sub2-7 (15 g, 59.4 mmol), amine10 (24 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 23.6 g of Compound 2-18. (Yield: 66%, MS: [M+H]⁺=602).

### Synthesis Example 2-19

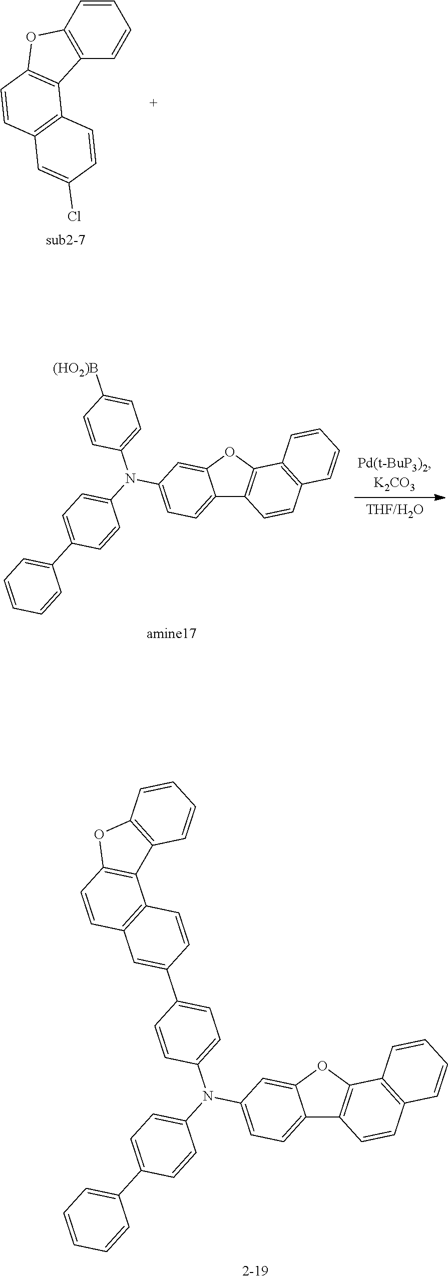

sub2-7 (15 g, 59.4 mmol) and amine17 (31.5 g, 62.3 mmol) were added to 300 ml of THF, and the mixture was stirred and refluxed. Then, potassium carbonate (24.6 g, 178.1 mmol) was dissolved in 100 mL of water, added thereto, sufficiently stirred, and then bis(tri-tert-butylphosphine)palladium(0) (0.3 g, 0.6 mmol) was added. After the reaction for 2 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 31 g of Compound 2-19. (Yield: 77%, MS: [M+H]⁺=678).

### Synthesis Example 2-20

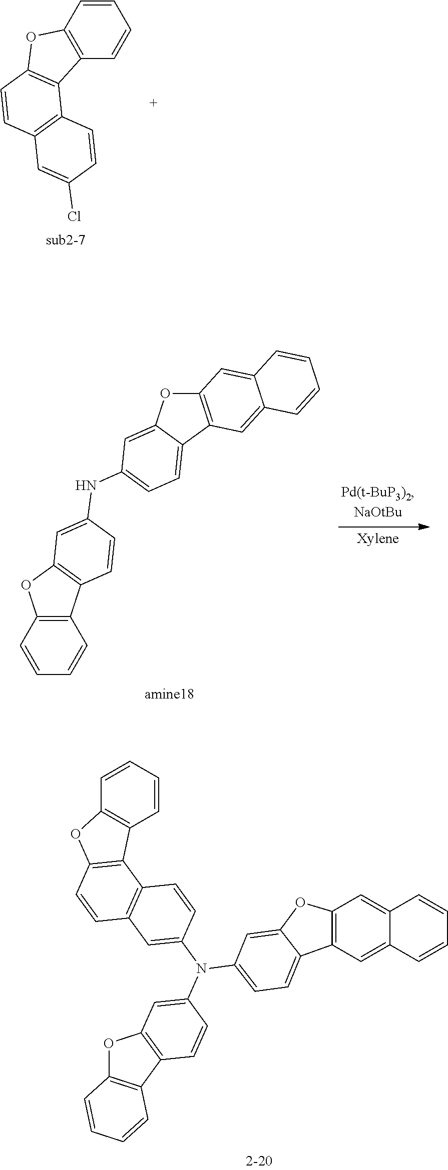 [SMILES: Clc1ccc2c(ccc3oc4ccccc4c32)c1 ; c1ccc2cc3c(cc2c1)oc1cc(Nc2ccc4c(c2)oc2ccccc24)ccc13 ; c1ccc2cc3c(cc2c1)oc1cc(N(c2ccc4c(ccc5oc6ccccc6c54)c2)c2ccc4c(c2)oc2ccccc24)ccc13]

sub2-7 (15 g, 59.4 mmol), amine18 (24.9 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 23 g of Compound 2-20. (Yield: 63%, MS: [M+H]⁺=616).

### Synthesis Example 2-21

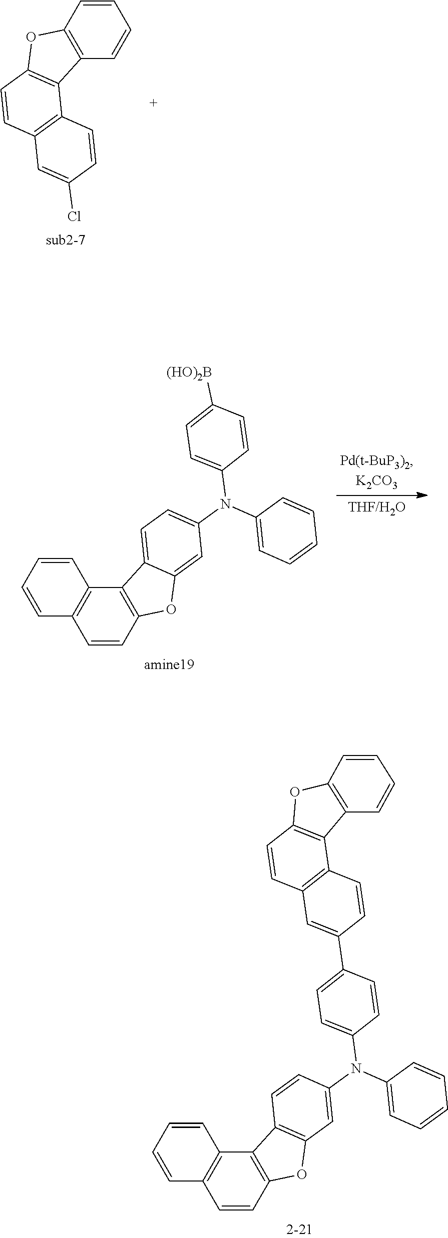 [SMILES: Clc1ccc2c(ccc3oc4ccccc4c32)c1 ; OB(O)c1ccc(N(c2ccccc2)c2ccc3c(c2)oc2ccc4ccccc4c23)cc1 ; c1ccc(N(c2ccc(-c3ccc4c(ccc5oc6ccccc6c54)c3)cc2)c2ccc3c(c2)oc2ccc4ccccc4c23)cc1]

sub2-7 (15 g, 59.4 mmol) and amine19 (26.8 g, 62.3 mmol) were added to 300 ml of THF, and the mixture was stirred and refluxed. Then, potassium carbonate (24.6 g, 178.1 mmol) was dissolved in 100 mL of water, added thereto, sufficiently stirred, and then bis(tri-tert-butylphosphine)palladium(0) (0.3 g, 0.6 mmol) was added. After the reaction for 2 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 23.9 g of Compound 2-21. (Yield: 67%, MS: [M+H]⁺=602).

### Synthesis Example 2-22

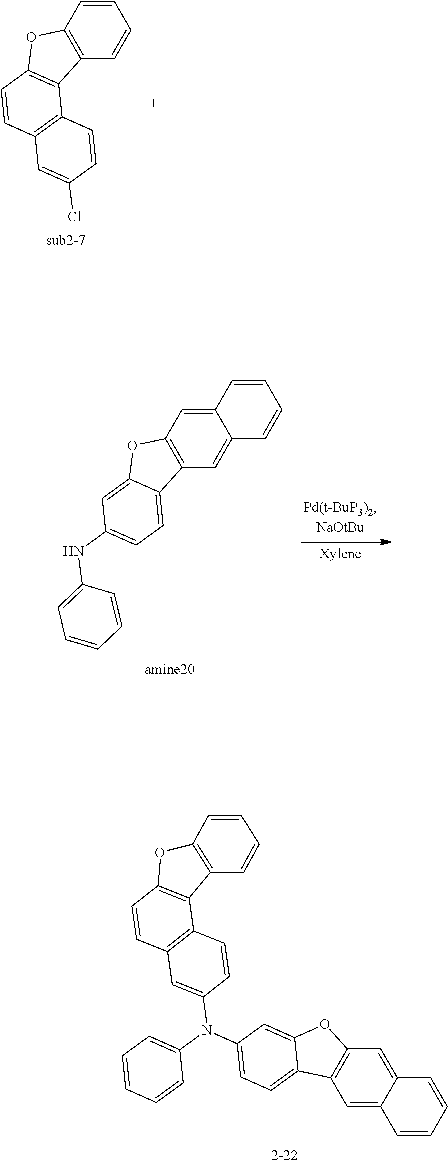 [SMILES: Clc1ccc2c(ccc3oc4ccccc4c32)c1 ; c1ccc(Nc2ccc3c(c2)oc2cc4ccccc4cc23)cc1 ; c1ccc(N(c2ccc3c(ccc4oc5ccccc5c43)c2)c2ccc3c(c2)oc2cc4ccccc4cc23)cc1]

sub2-7 (15 g, 59.4 mmol), amine20 (19.3 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 18.7 g of Compound 2-22. (Yield: 60%, MS: [M+H]⁺=526).

### Synthesis Example 2-23

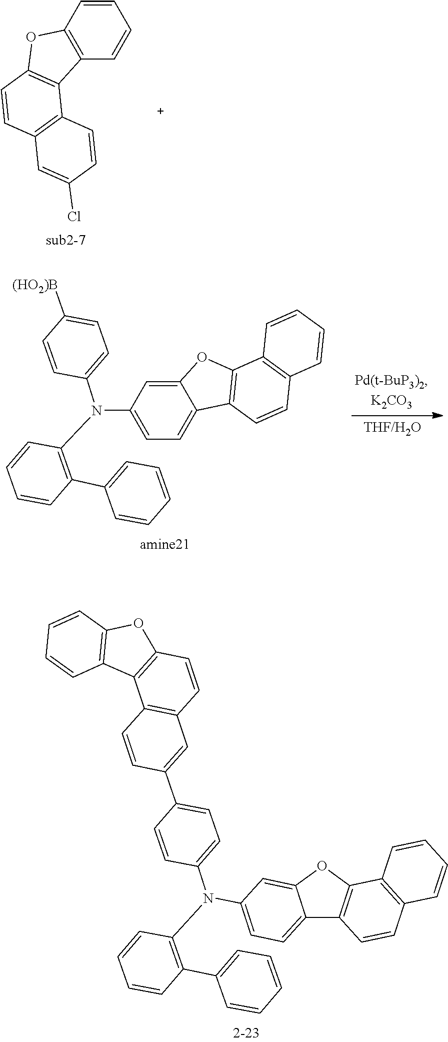

sub2-7 (15 g, 59.4 mmol) and amine21 (31.5 g, 62.3 mmol) were added to 300 ml of THF, and the mixture was stirred and refluxed. Then, potassium carbonate (24.6 g, 178.1 mmol) was dissolved in 100 mL of water, added thereto, sufficiently stirred, and then bis(tri-tert-butylphosphine)palladium(0) (0.3 g, 0.6 mmol) was added. After the reaction for 3 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 28.1 g of Compound 2-23. (Yield: 70%, MS: [M+H]⁺=678).

### Synthesis Example 2-24

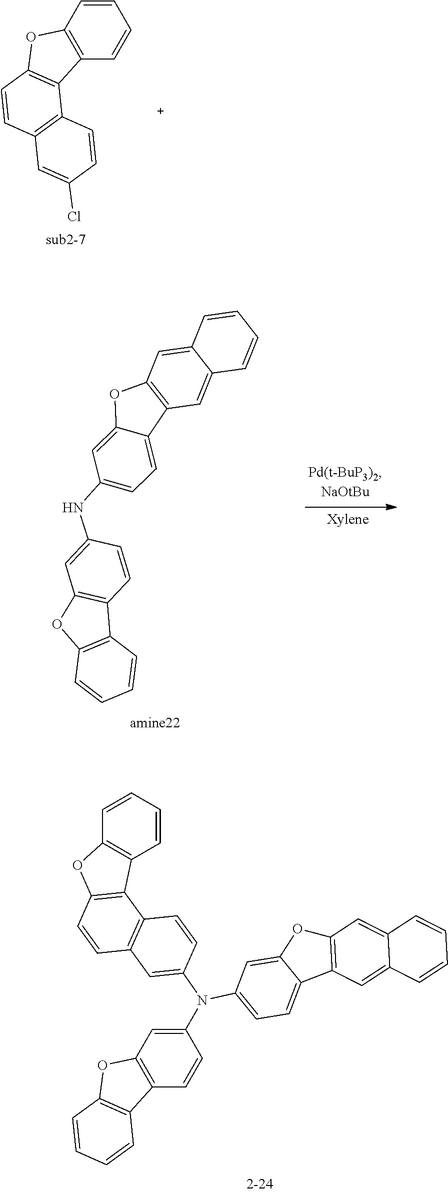 [SMILES: Clc1ccc2c(ccc3oc4ccccc4c32)c1 ; c1ccc2cc3c(cc2c1)oc1cc(Nc2ccc4c(c2)oc2ccccc24)ccc13 ; c1ccc2cc3c(cc2c1)oc1cc(N(c2ccc4c(ccc5oc6ccccc6c54)c2)c2ccc4c(c2)oc2ccccc24)ccc13]

sub2-7 (15 g, 59.4 mmol), amine22 (24.9 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 21.5 g of Compound 2-24. (Yield: 59%, MS: [M+H]⁺=616).

### Synthesis Example 2-25

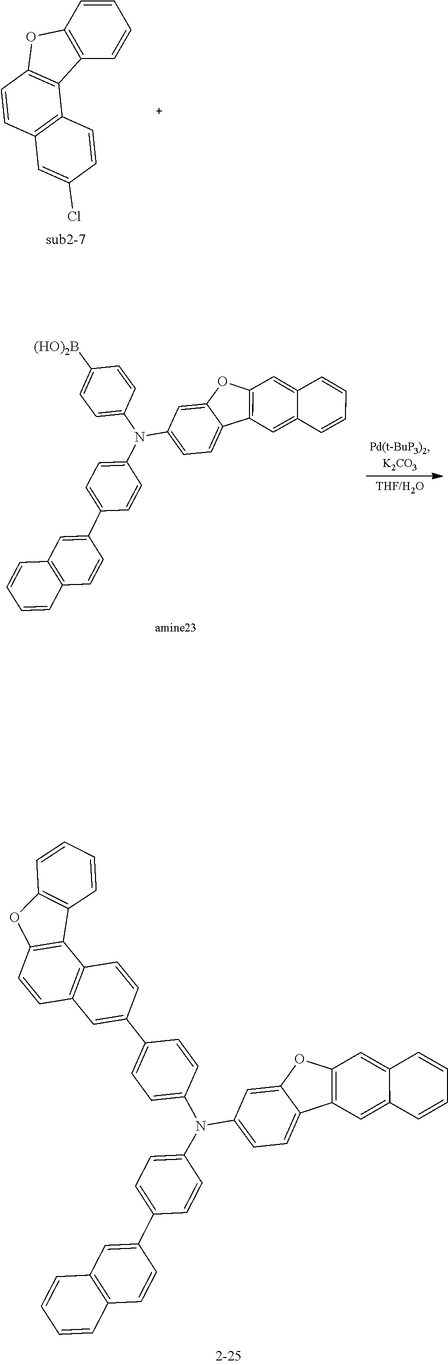 [SMILES: Clc1ccc2c(ccc3oc4ccccc4c32)c1 ; OB(O)c1ccc(N(c2ccc(-c3ccc4ccccc4c3)cc2)c2ccc3c(c2)oc2cc4ccccc4cc23)cc1 ; c1ccc2cc(-c3ccc(N(c4ccc(-c5ccc6c(ccc7oc8ccccc8c76)c5)cc4)c4ccc5c(c4)oc4cc6ccccc6cc45)cc3)ccc2c1]

sub2-7 (15 g, 59.4 mmol) and amine23 (34.6 g, 62.3 mmol) were added to 300 ml of THF, and the mixture was stirred and refluxed. Then, potassium carbonate (24.6 g, 178.1 mmol) was dissolved in 100 mL of water, added thereto, sufficiently stirred, and then bis(tri-tert-butylphosphine)palladium(0) (0.3 g, 0.6 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 26.8 g of Compound 2-25. (Yield: 62%, MS: [M+H]⁺=728).

### Synthesis Example 2-26

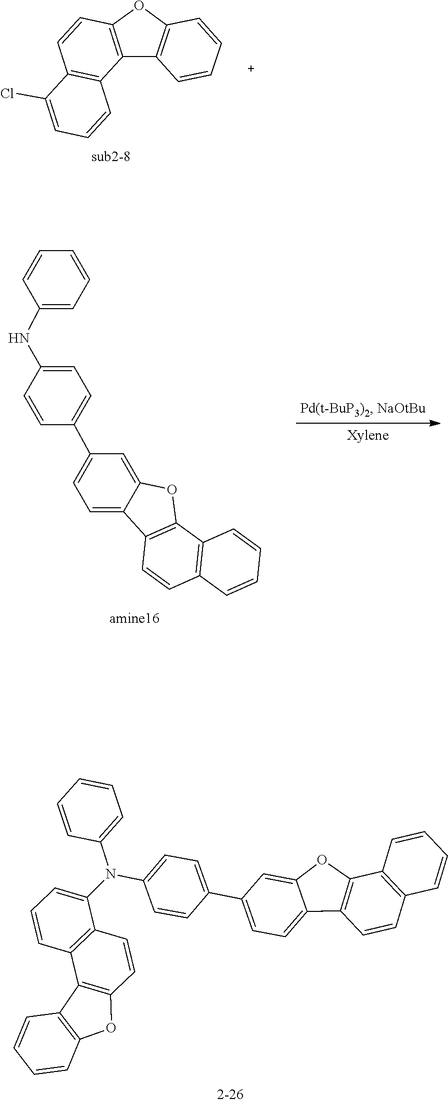 [SMILES: Clc1cccc2c1ccc1oc3ccccc3c12 ; c1ccc(Nc2ccc(-c3ccc4c(c3)oc3c5ccccc5ccc43)cc2)cc1 ; c1ccc(N(c2ccc(-c3ccc4c(c3)oc3c5ccccc5ccc43)cc2)c2cccc3c2ccc2oc4ccccc4c23)cc1]

sub2-8 (15 g, 59.4 mmol), amine16 (24 g, 62.3 mmol) and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 23.2 g of Compound 2-26. (Yield: 65%, MS: [M+H]⁺=602).

### Synthesis Example 2-27

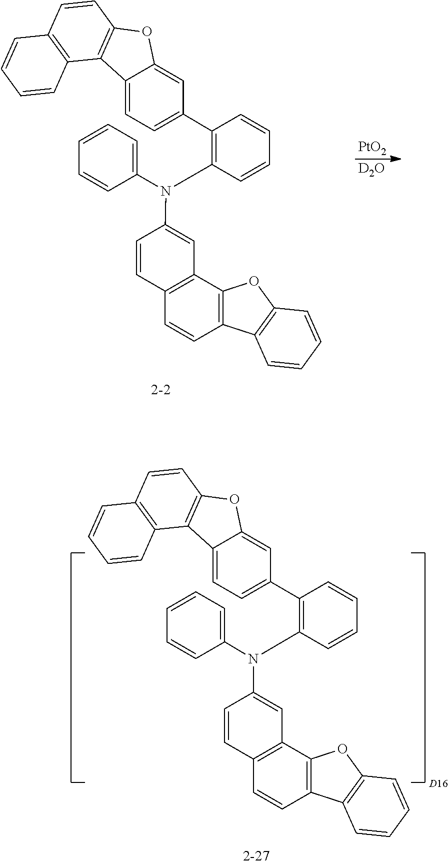

Compound 2-2 (10 g, 16.6 mmol), PtO₂ (1.1 g, 5 mmol) and D₂O (83 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 5.2 g of Compound 2-27. At this time, the deuterium substitution rate was calculated in the same manner as in Compound 1-56. (Yield: 51%, M.W.=614)

### Synthesis Example 2-28

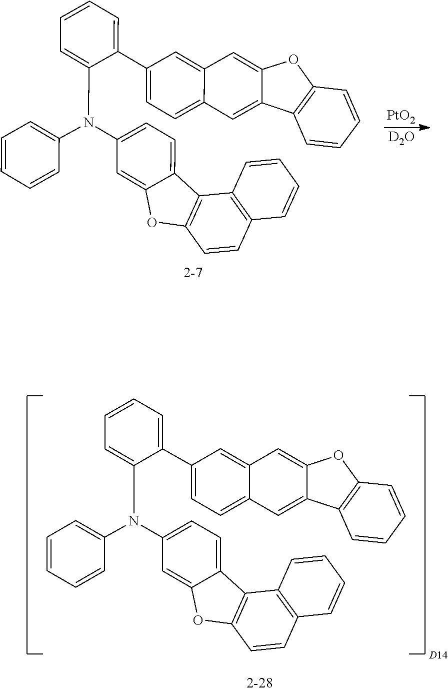

Compound 2-7 (10 g, 16.6 mmol), PtO₂ (1.1 g, 5 mmol) and D₂O (83 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 4.3 g of Compound 2-28. At this time, the deuterium substitution rate was calculated in the same manner as in Compound 1-56. (Yield: 42%, M.W.=615)

### Synthesis Example 2-29

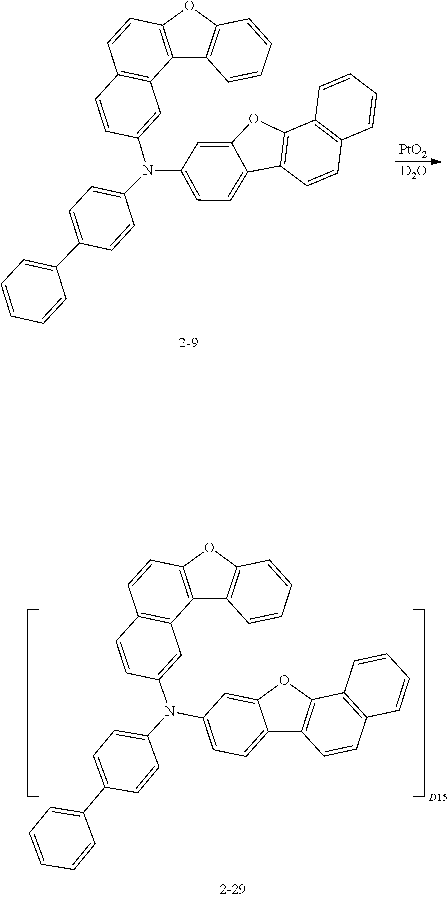

Compound 2-9 (10 g, 16.6 mmol), PtO₂ (1.1 g, 5 mmol) and D₂O (83 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 4.7 g of Compound 2-29. At this time, the deuterium substitution rate was calculated in the same manner as in Compound 1-56. (Yield: 46%, M.W.=616)

### Synthesis Example 2-30

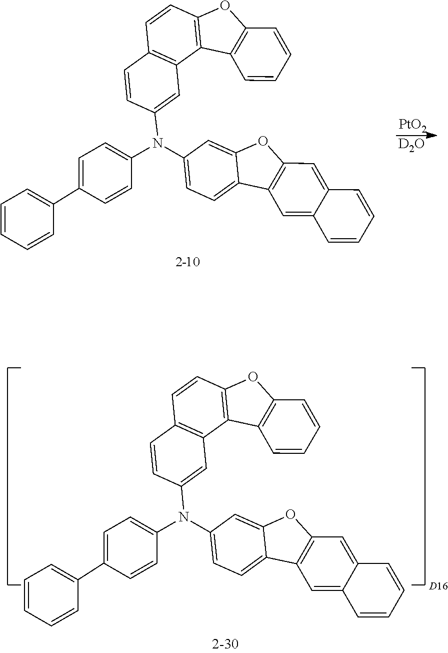

Compound 2-10 (10 g, 16.6 mmol), PtO₂ (1.1 g, 5 mmol) and D₂O (83 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 5.3 g of Compound 2-30. At this time, the deuterium substitution rate was calculated in the same manner as in Compound 1-56. (Yield: 52%, M.W.=617)

### Synthesis Example 2-31

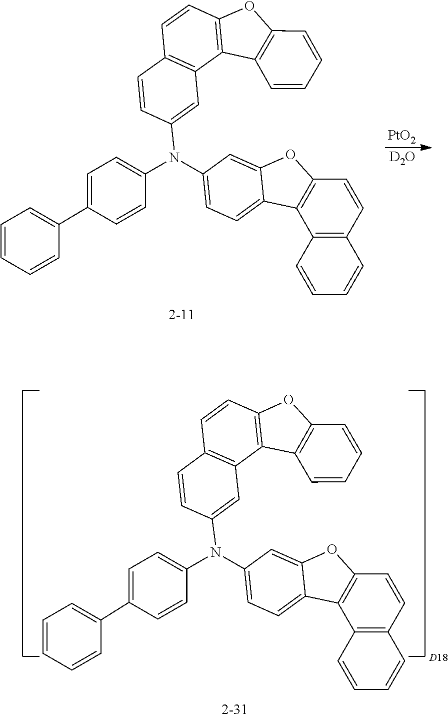

Compound 2-11 (10 g, 16.6 mmol), PtO₂ (1.1 g, 5 mmol) and D₂O (83 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 4.3 g of Compound 2-31. At this time, the deuterium substitution rate was calculated in the same manner as in Compound 1-56. (Yield: 42%, M.W.=619)

### Synthesis Example 2-32

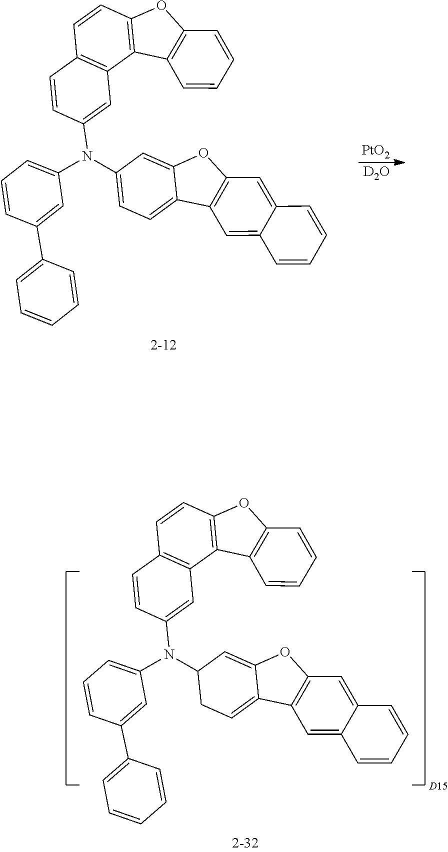

Compound 2-12 (10 g, 16.6 mmol), PtO₂ (1.1 g, 5 mmol) and D₂O (83 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 5.6 g of Compound 2-32. At this time, the deuterium substitution rate was calculated in the same manner as in Compound 1-56. (Yield: 55%, M.W.=616)

### Synthesis Example 2-33

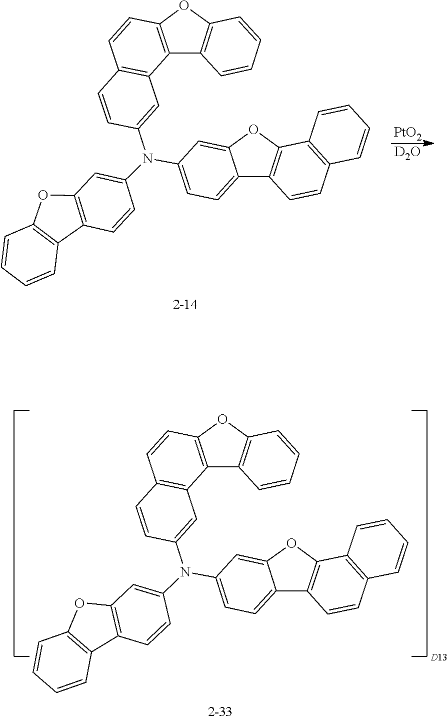

Compound 2-14 (10 g, 16.2 mmol), PtO₂ (1.1 g, 4.9 mmol) and D₂O (81 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 5.6 g of Compound 2-33. At this time, the deuterium substitution rate was calculated in the same manner as in Compound 1-56. (Yield: 55%, M.W.=628)

### Synthesis Example 2-34

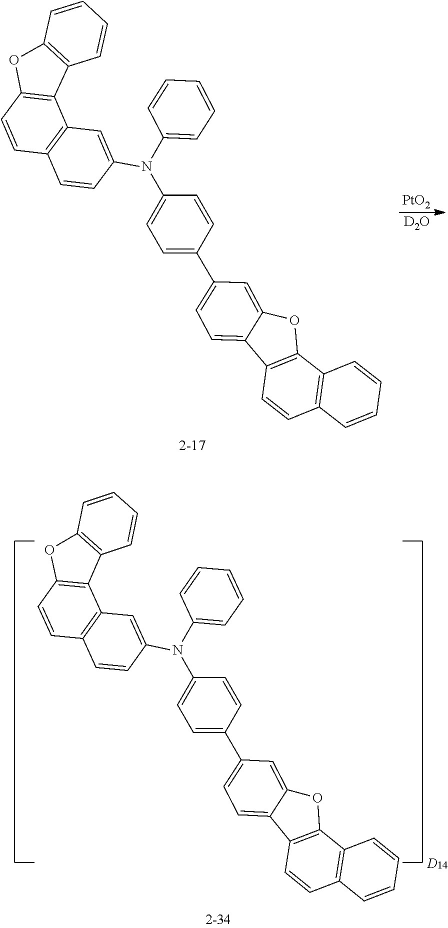

Compound 2-17 (10 g, 16.6 mmol), PtO₂ (1.1 g, 5 mmol) and D₂O (83 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 5.6 g of Compound 2-34. At this time, the deuterium substitution rate was calculated in the same manner as in Compound 1-56. (Yield: 55%, M.W.=615)

### Synthesis Example 2-35

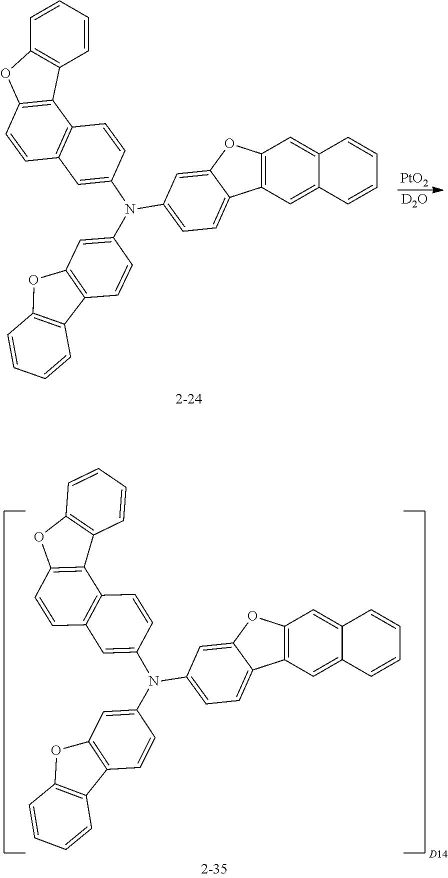

Compound 2-24 (10 g, 16.2 mmol), PtO₂ (1.1 g, 4.9 mmol) and D₂O (81 mL) were added to a shaker tube, and then the tube was sealed and heated at 250° C. and 600 psi for 12 hours. When the reaction was terminated, chloroform was added, and the reaction solution was transferred to a separatory funnel, and extracted. The extract was dried over MgSO₄ and concentrated, and then the sample was purified by a silica gel column chromatography to prepare 4.4 g of Compound 2-35. At this time, the deuterium substitution rate was calculated in the same manner as in Compound 1-56. (Yield: 43%, M.W.=629)

### Example 1

A glass substrate on which ITO (indium tin oxide) was coated as a thin film to a thickness of 1,000 Å was put into distilled water in which a detergent was dissolved, and ultrasonically cleaned. A product manufactured by Fischer Co. was used as the detergent, and as the distilled water, distilled water filtered twice using a filter manufactured by Millipore Co. was used. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. In addition, the substrate was cleaned for 5 minutes using oxygen plasma and then transferred to a vacuum depositor.

On the ITO transparent electrode thus prepared, the following compound HI-1 was formed to a thickness of 1150 Å as a hole injection layer, but the following compound A-1 was p-doped at a concentration of 1.5 wt. %. The following compound HT-1 was vacuum deposited on the hole injection layer to form a hole transport layer with a layer thickness of 800 Å. Then, the following compound EB-1 was vacuum deposited on the hole transport layer to a layer thickness of 150 Å to form an electron blocking layer.

Then, Compound 1-1 of Synthesis Example 1-1, Compound 2-1 of Synthesis Example 2-1, and the following Compound Dp-7 were vacuum deposited in a weight ratio of 49:49:2 on the EB-1 deposited layer to form a red light emitting layer with a layer thickness of 400 Å.

The following compound HB-1 was vacuum deposited on the light emitting layer to a layer thickness of 30 Å to form a hole blocking layer. Then, the following compound ET-1 and the following compound LiQ were vacuum deposited in a weight ratio of 2:1 on the hole blocking layer to form an electron injection and transport layer with a layer thickness of 300 Å.

Lithium fluoride (LiF) and aluminum were sequentially deposited to have a thickness of 12 Å and 1,000 Å, respectively, on the electron injection and transport layer, thereby forming a cathode.

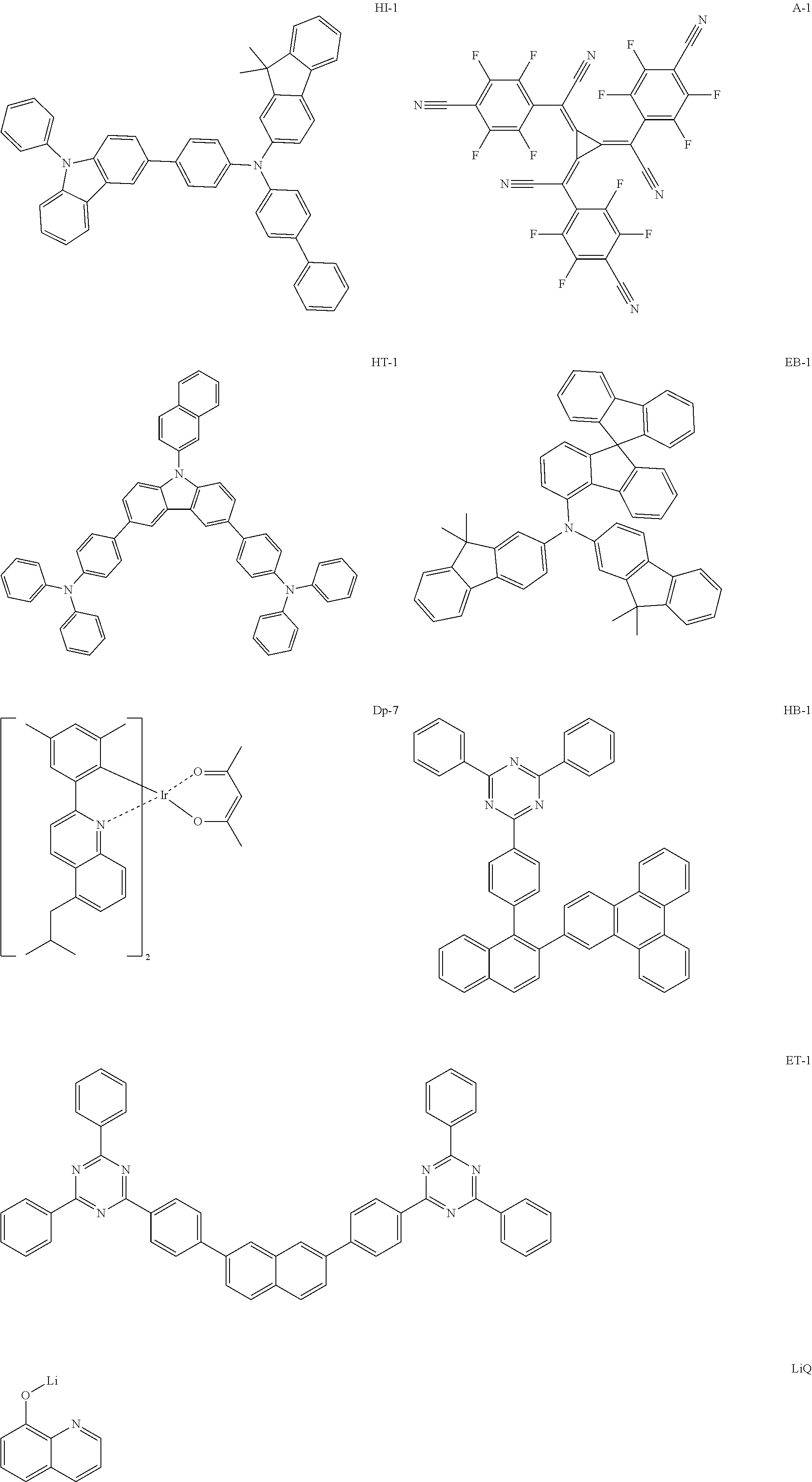

In the above-mentioned processes, the vapor deposition rate of the organic material was maintained at 0.4˜0.7 Å/sec, the deposition rates of lithium fluoride and aluminum of the cathode were maintained at 0.3 Å/sec and 2 Å/sec, respectively, and the degree of vacuum during the deposition was maintained at 2×10⁻⁷˜5×10⁻⁶ torr, thereby manufacturing an organic light emitting device.

### Example 2 to Example 190

The organic light emitting device was manufactured in the same manner as in Example 1, except that in the organic light emitting device of Example 1, the first host and the second host shown in Tables 1 to 5 below were co-deposited and used in a ratio of 1:1 instead of Compound 1-1 and Compound 2-1.

### Comparative Example 1 to Comparative Example 12

The organic light emitting device was manufactured in the same manner as in Example 1, except that in the organic light emitting device of Example 1, the first host and the second host shown in Table 6 below were co-deposited and used in a ratio of 1:1 instead of Compound 1-1 and Compound 2-1. At this time, the structures of comparative compounds E-1 to B-4 used in Comparative Examples are summarized as follows.

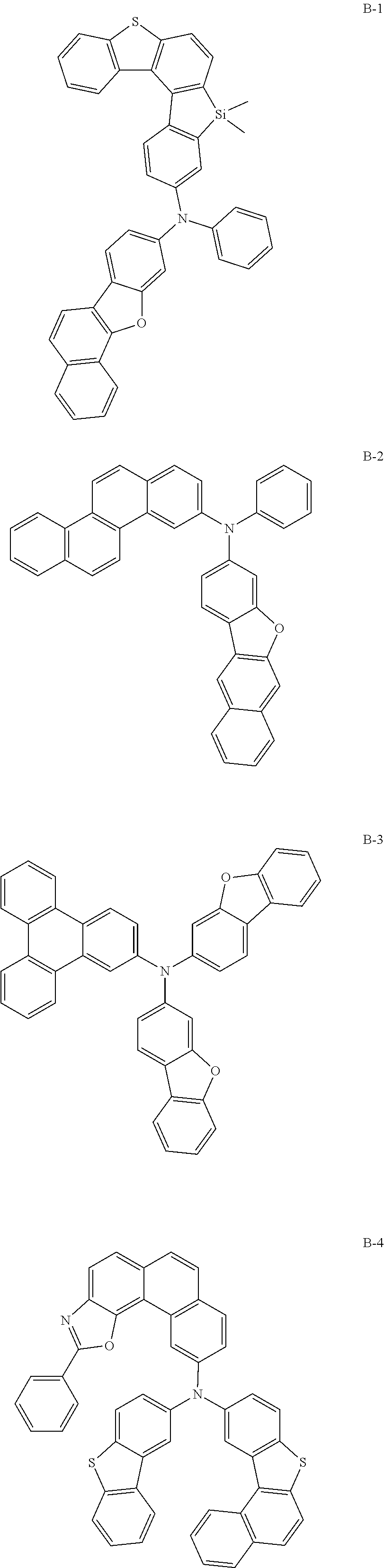 [SMILES: C[Si]1(C)c2cc(N(c3ccccc3)c3ccc4c(c3)oc3c5ccccc5ccc43)ccc2-c2c1ccc1sc3ccccc3c21 ; c1ccc(N(c2ccc3c(c2)oc2cc4ccccc4cc23)c2ccc3ccc4c5ccccc5ccc4c3c2)cc1 ; c1ccc2c(c1)oc1cc(N(c3ccc4c(c3)oc3ccccc34)c3ccc4c5ccccc5c5ccccc5c4c3)ccc12 ; c1ccc(-c2nc3ccc4ccc5ccc(N(c6ccc7sc8ccccc8c7c6)c6ccc7sc8ccc9ccccc9c8c7c6)cc5c4c3o2)cc1]

### Experimental Example 1: Evaluation of Device Characteristics

The voltage, efficiency and lifetime were measured by applying a current to the organic light emitting devices manufactured in the Examples 1 to 190 and Comparative Examples 1 to 12, and the results are shown in Tables 1 to 5 below. At this time, the lifetime T95 was measured based on 7000 nit, and T95 means the time required for the lifetime to be reduced to 95% of the initial lifetime.

TABLE 1

| | | | Driving | | | |
|---|---|---|---|---|---|---|
| | First | Second | voltage | Efficiency | Lifetime | Luminous |
| Category | host | host | (V) | (cd/A) | T95(hr) | color |
| Example 1 | Compound | Compound | 3.60 | 22.61 | 278 | Red |
| | 1-1 | 2-1 | | | | |
| Example 2 | | Compound | 3.56 | 22.51 | 215 | Red |
| | | 2-7 | | | | |
| Example 3 | | Compound | 3.61 | 22.26 | 223 | Red |
| | | 2-15 | | | | |
| Example 4 | | Compound | 3.53 | 22.62 | 215 | Red |
| | | 2-21 | | | | |
| Example 5 | | Compound | 3.65 | 22.45 | 247 | Red |
| | | 2-28 | | | | |
| Example 6 | Compound | Compound | 3.52 | 21.62 | 265 | Red |
| | 1-2 | 2-2 | | | | |
| Example 7 | | Compound | 3.51 | 21.71 | 241 | Red |
| | | 2-6 | | | | |
| Example 8 | | Compound | 3.56 | 21.51 | 226 | Red |
| | | 2-13 | | | | |
| Example 9 | | Compound | 3.69 | 21.47 | 295 | Red |
| | | 2-22 | | | | |
| Example 10 | | Compound | 3.64 | 21.23 | 223 | Red |
| | | 2-35 | | | | |
| Example 11 | Compound | Compound | 3.51 | 21.69 | 215 | Red |
| | 1-5 | 2-3 | | | | |
| Example 12 | | Compound | 3.55 | 21.74 | 226 | Red |
| | | 2-8 | | | | |
| Example 13 | | Compound | 3.61 | 21.77 | 287 | Red |
| | | 2-16 | | | | |
| Example 14 | | Compound | 3.55 | 22.79 | 215 | Red |
| | | 2-26 | | | | |
| Example 15 | | Compound | 3.52 | 22.15 | 226 | Red |
| | | 2-30 | | | | |
| Example 16 | Compound | Compound | 3.73 | 20.99 | 287 | Red |
| | 1-6 | 2-4 | | | | |
| Example 17 | | Compound | 3.76 | 20.84 | 215 | Red |
| | | 2-11 | | | | |
| Example 18 | | Compound | 3.60 | 20.75 | 265 | Red |
| | | 2-19 | | | | |
| Example 19 | | Compound | 3.68 | 20.61 | 298 | Red |
| | | 2-23 | | | | |
| Example 20 | | Compound | 3.61 | 21.23 | 248 | Red |
| | | 2-31 | | | | |
| Example 21 | Compound | Compound | 3.79 | 20.15 | 223 | Red |
| | 1-7 | 2-5 | | | | |
| Example 22 | | Compound | 3.71 | 20.15 | 230 | Red |
| | | 2-8 | | | | |
| Example 23 | | Compound | 3.70 | 20.49 | 278 | Red |
| | | 2-18 | | | | |
| Example 24 | | Compound | 3.73 | 21.78 | 251 | Red |
| | | 2-27 | | | | |
| Example 25 | | Compound | 3.76 | 20.79 | 295 | Red |
| | | 2-32 | | | | |
| Example 26 | Compound | Compound | 3.41 | 23.15 | 302 | Red |
| | 1-9 | 2-2 | | | | |
| Example 27 | | Compound | 3.45 | 23.65 | 312 | Red |
| | | 2-12 | | | | |
| Example 28 | | Compound | 3.44 | 22.55 | 298 | Red |
| | | 2-14 | | | | |
| Example 29 | | Compound | 3.42 | 22.34 | 278 | Red |
| | | 2-20 | | | | |
| Example 30 | | Compound | 3.48 | 22.54 | 305 | Red |
| | | 2-25 | | | | |
| Example 31 | Compound | Compound | 3.54 | 23.14 | 360 | Red |
| | 1-10 | 2-3 | | | | |
| Example 32 | | Compound | 3.42 | 22.54 | 315 | Red |
| | | 2-15 | | | | |
| Example 33 | | Compound | 3.40 | 22.97 | 348 | Red |
| | | 2-17 | | | | |
| Example 34 | | Compound | 3.49 | 23.17 | 387 | Red |
| | | 2-24 | | | | |
| Example 35 | | Compound | 3.41 | 23.62 | 315 | Red |
| | | 2-29 | | | | |
| Example 36 | Compound | Compound | 3.52 | 22.28 | 298 | Red |
| | 1-12 | 2-5 | | | | |
| Example 37 | | Compound | 3.43 | 22.47 | 345 | Red |
| | | 2-13 | | | | |
| Example 38 | | Compound | 3.46 | 22.13 | 321 | Red |
| | | 2-20 | | | | |
| Example 39 | | Compound | 3.41 | 22.64 | 330 | Red |
| | | 2-28 | | | | |
| Example 40 | | Compound | 3.45 | 22.14 | 387 | Red |
| | | 2-33 | | | | |

TABLE 2

| | | | Driving | | | |
|---|---|---|---|---|---|---|
| | First | Second | voltage | Efficiency | Lifetime | Luminous |
| Category | host | host | (V) | (cd/A) | T95(hr) | color |
| Example 41 | Compound | Compound | 3.68 | 20.15 | 287 | Red |
| | 1-14 | 2-1 | | | | |
| Example 42 | | Compound | 3.74 | 20.14 | 298 | Red |
| | | 2-12 | | | | |
| Example 43 | | Compound | 3.72 | 20.16 | 256 | Red |
| | | 2-19 | | | | |
| Example 44 | | Compound | 3.63 | 21.26 | 267 | Red |
| | | 2-26 | | | | |
| Example 45 | | Compound | 3.6 | 20.23 | 248 | Red |
| | | 2-34 | | | | |
| Example 46 | Compound | Compound | 3.62 | 20.48 | 291 | Red |
| | 1-16 | 2-4 | | | | |
| Example 47 | | Compound | 3.72 | 21.46 | 260 | Red |
| | | 2-14 | | | | |
| Example 48 | | Compound | 3.70 | 20.11 | 284 | Red |
| | | 2-21 | | | | |
| Example 49 | | Compound | 3.71 | 20.48 | 298 | Red |
| | | 2-29 | | | | |
| Example 50 | | Compound | 3.72 | 20.79 | 260 | Red |
| | | 2-35 | | | | |
| Example 51 | Compound | Compound | 3.53 | 22.20 | 287 | Red |
| | 1-20 | 2-6 | | | | |
| Example 52 | | Compound | 3.56 | 22.12 | 291 | Red |
| | | 2-16 | | | | |
| Example 53 | | Compound | 3.51 | 22.14 | 264 | Red |
| | | 2-23 | | | | |
| Example 54 | | Compound | 3.68 | 22.79 | 266 | Red |
| | | 2-33 | | | | |
| Example 55 | | Compound | 3.64 | 22.25 | 289 | Red |
| | | 2-34 | | | | |
| Example 56 | Compound | Compound | 3.52 | 22.20 | 290 | Red |
| | 1-21 | 2-7 | | | | |
| Example 57 | | Compound | 3.53 | 22.15 | 278 | Red |
| | | 2-17 | | | | |
| Example 58 | | Compound | 3.67 | 22.64 | 265 | Red |
| | | 2-18 | | | | |
| Example 59 | | Compound | 3.65 | 22.47 | 245 | Red |
| | | 2-24 | | | | |
| Example 60 | | Compound | 3.56 | 22.26 | 212 | Red |
| | | 2-32 | | | | |
| Example 61 | Compound | Compound | 3.60 | 22.94 | 290 | Red |
| | 1-22 | 2-3 | | | | |
| Example 62 | | Compound | 3.59 | 21.14 | 245 | Red |
| | | 2-13 | | | | |
| Example 63 | | Compound | 3.53 | 21.16 | 230 | Red |
| | | 2-22 | | | | |
| Example 64 | | Compound | 3.62 | 21.27 | 265 | Red |
| | | 2-27 | | | | |
| Example 65 | | Compound | 3.63 | 21.14 | 248 | Red |
| | | 2-34 | | | | |
| Example 66 | Compound | Compound | 3.62 | 21.65 | 251 | Red |
| | 1-24 | 2-2 | | | | |
| Example 67 | | Compound | 3.66 | 21.37 | 261 | Red |
| | | 2-12 | | | | |
| Example 68 | | Compound | 3.51 | 21.19 | 248 | Red |
| | | 2-18 | | | | |
| Example 69 | | Compound | 3.55 | 21.37 | 235 | Red |
| | | 2-25 | | | | |
| Example 70 | | Compound | 3.5 | 21.23 | 298 | Red |
| | | 2-33 | | | | |
| Example 71 | Compound | Compound | 3.71 | 20.85 | 251 | Red |
| | 1-26 | 2-1 | | | | |
| Example 72 | | Compound | 3.63 | 20.46 | 230 | Red |
| | | 2-10 | | | | |
| Example 73 | | Compound | 3.71 | 20.49 | 287 | Red |
| | | 2-21 | | | | |
| Example 74 | | Compound | 3.61 | 20.47 | 215 | Red |
| | | 2-30 | | | | |
| Example 75 | | Compound | 3.62 | 20.97 | 260 | Red |
| | | 2-35 | | | | |
| Example 76 | Compound | Compound | 3.60 | 20.14 | 278 | Red |
| | 1-27 | 2-4 | | | | |
| Example 77 | | Compound | 3.73 | 20.96 | 298 | Red |
| | | 2-15 | | | | |
| Example 78 | | Compound | 3.6 | 20.55 | 215 | Red |
| | | 2-22 | | | | |
| Example 79 | | Compound | 3.69 | 20.97 | 226 | Red |
| | | 2-32 | | | | |
| Example 80 | | Compound | 3.65 | 20.17 | 203 | Red |
| | | 2-35 | | | | |

TABLE 3

| | | | Driving | | | |
|---|---|---|---|---|---|---|
| | First | Second | voltage | Efficiency | Lifetime | Luminous |
| Category | host | host | (V) | (cd/A) | T95(hr) | color |
| Example 81 | Compound | Compound | 3.55 | 21.37 | 287 | Red |
| | 1-29 | 2-5 | | | | |
| Example 82 | | Compound | 3.53 | 21.94 | 215 | Red |
| | | 2-9 | | | | |
| Example 83 | | Compound | 3.51 | 21.57 | 260 | Red |
| | | 2-14 | | | | |
| Example 84 | | Compound | 3.55 | 21.16 | 294 | Red |
| | | 2-23 | | | | |
| Example 85 | | Compound | 3.52 | 21.47 | 231 | Red |
| | | 2-32 | | | | |
| Example 86 | Compound | Compound | 3.56 | 23.61 | 345 | Red |
| | 1-30 | 2-6 | | | | |
| Example 87 | | Compound | 3.49 | 22.23 | 387 | Red |
| | | 2-11 | | | | |
| Example 88 | | Compound | 3.51 | 22.14 | 315 | Red |
| | | 2-16 | | | | |
| Example 89 | | Compound | 3.42 | 22.24 | 326 | Red |
| | | 2-27 | | | | |
| Example 90 | | Compound | 3.40 | 22.52 | 330 | Red |
| | | 2-30 | | | | |
| Example 91 | Compound | Compound | 3.43 | 22.71 | 371 | Red |
| | 1-33 | 2-7 | | | | |
| Example 92 | | Compound | 3.41 | 22.47 | 312 | Red |
| | | 2-17 | | | | |
| Example 93 | | Compound | 3.48 | 22.96 | 345 | Red |
| | | 2-20 | | | | |
| Example 94 | | Compound | 3.41 | 22.48 | 360 | Red |
| | | 2-26 | | | | |
| Example 95 | | Compound | 3.55 | 22.08 | 311 | Red |
| | | 2-29 | | | | |
| Example 96 | Compound | Compound | 3.62 | 22.34 | 203 | Red |
| | 1-36 | 2-8 | | | | |
| Example 97 | | Compound | 3.50 | 22.07 | 265 | Red |
| | | 2-15 | | | | |
| Example 98 | | Compound | 3.66 | 22.62 | 278 | Red |
| | | 2-19 | | | | |
| Example 99 | | Compound | 3.61 | 22.79 | 294 | Red |
| | | 2-25 | | | | |
| Example 100 | | Compound | 3.62 | 22.14 | 215 | Red |
| | | 2-28 | | | | |
| Example 101 | Compound | Compound | 3.63 | 22.67 | 260 | Red |
| | 1-37 | 2-1 | | | | |
| Example 102 | | Compound | 3.64 | 22.26 | 278 | Red |
| | | 2-10 | | | | |
| Example 103 | | Compound | 3.5 | 22.37 | 260 | Red |
| | | 2-22 | | | | |
| Example 104 | | Compound | 3.55 | 22.14 | 231 | Red |
| | | 2-24 | | | | |
| Example 105 | | Compound | 3.51 | 22.62 | 245 | Red |
| | | 2-31 | | | | |
| Example 106 | Compound | Compound | 3.4 | 22.67 | 315 | Red |
| | 1-39 | 2-4 | | | | |
| Example 107 | | Compound | 3.49 | 22.14 | 321 | Red |
| | | 2-11 | | | | |
| Example 108 | | Compound | 3.51 | 22.97 | 306 | Red |
| | | 2-20 | | | | |
| Example 109 | | Compound | 3.44 | 22.15 | 380 | Red |
| | | 2-30 | | | | |
| Example 110 | | Compound | 3.43 | 22.23 | 394 | Red |
| | | 2-31 | | | | |
| Example 111 | Compound | Compound | 3.57 | 21.47 | 298 | Red |
| | 1-42 | 2-5 | | | | |
| Example 112 | | Compound | 3.60 | 21.48 | 260 | Red |
| | | 2-13 | | | | |
| Example 113 | | Compound | 3.51 | 21.67 | 215 | Red |
| | | 2-18 | | | | |
| Example 114 | | Compound | 3.55 | 22.85 | 244 | Red |
| | | 2-22 | | | | |
| Example 115 | | Compound | 3.62 | 21.44 | 215 | Red |
| | | 2-34 | | | | |
| Example 116 | Compound | Compound | 3.63 | 22.23 | 278 | Red |
| | 1-43 | 2-2 | | | | |
| Example 117 | | Compound | 3.60 | 21.46 | 245 | Red |
| | | 2-9 | | | | |
| Example 118 | | Compound | 3.57 | 21.47 | 216 | Red |
| | | 2-14 | | | | |
| Example 119 | | Compound | 3.57 | 21.19 | 245 | Red |
| | | 2-21 | | | | |
| Example 120 | | Compound | 3.61 | 21.67 | 297 | Red |
| | | 2-33 | | | | |

TABLE 4

| | | | Driving | | | |
|---|---|---|---|---|---|---|
| | First | Second | voltage | Efficiency | Lifetime | Luminous |
| Category | host | host | (V) | (cd/A) | T95(hr) | color |
| Example 121 | Compound | Compound | 3.66 | 20.67 | 215 | Red |
| | 1-44 | 2-3 | | | | |
| Example 122 | | Compound | 3.42 | 20.41 | 232 | Red |
| | | 2-8 | | | | |
| Example 123 | | Compound | 3.61 | 20.27 | 212 | Red |
| | | 2-12 | | | | |
| Example 124 | | Compound | 3.40 | 20.26 | 260 | Red |
| | | 2-25 | | | | |
| Example 125 | | Compound | 3.52 | 20.14 | 279 | Red |
| | | 2-33 | | | | |
| Example 126 | Compound | Compound | 3.53 | 21.15 | 245 | Red |
| | 1-45 | 2-6 | | | | |
| Example 127 | | Compound | 3.51 | 21.31 | 278 | Red |
| | | 2-17 | | | | |
| Example 128 | | Compound | 3.55 | 21.65 | 260 | Red |
| | | 2-24 | | | | |
| Example 129 | | Compound | 3.56 | 22.67 | 297 | Red |
| | | 2-29 | | | | |
| Example 130 | | Compound | 3.51 | 21.94 | 250 | Red |
| | | 2-32 | | | | |
| Example 131 | Compound | Compound | 3.44 | 22.48 | 312 | Red |
| | 1-46 | 2-9 | | | | |
| Example 132 | | Compound | 3.48 | 23.15 | 332 | Red |
| | | 2-10 | | | | |
| Example 133 | | Compound | 3.52 | 23.37 | 315 | Red |
| | | 2-11 | | | | |
| Example 134 | | Compound | 3.43 | 23.26 | 360 | Red |
| | | 2-30 | | | | |
| Example 135 | | Compound | 3.43 | 23.77 | 378 | Red |
| | | 2-31 | | | | |
| Example 136 | Compound | Compound | 3.41 | 22.79 | 350 | Red |
| | 1-47 | 2-7 | | | | |
| Example 137 | | Compound | 3.43 | 23.24 | 301 | Red |
| | | 2-16 | | | | |
| Example 138 | | Compound | 3.41 | 22.65 | 298 | Red |
| | | 2-23 | | | | |
| Example 139 | | Compound | 3.45 | 22.15 | 365 | Red |
| | | 2-29 | | | | |
| Example 140 | | Compound | 3.42 | 22.67 | 387 | Red |
| | | 2-34 | | | | |
| Example 141 | Compound | Compound | 3.53 | 22.94 | 215 | Red |
| | 1-49 | 2-3 | | | | |
| Example 142 | | Compound | 3.56 | 21.14 | 232 | Red |
| | | 2-10 | | | | |
| Example 143 | | Compound | 3.41 | 21.46 | 260 | Red |
| | | 2-19 | | | | |
| Example 144 | | Compound | 3.45 | 21.18 | 247 | Red |
| | | 2-24 | | | | |
| Example 145 | | Compound | 3.59 | 22.14 | 245 | Red |
| | | 2-28 | | | | |
| Example 146 | Compound | Compound | 3.54 | 21.31 | 206 | Red |
| | 1-50 | 2-1 | | | | |
| Example 147 | | Compound | 3.52 | 21.16 | 297 | Red |
| | | 2-9 | | | | |
| Example 148 | | Compound | 3.53 | 21.17 | 215 | Red |
| | | 2-20 | | | | |
| Example 149 | | Compound | 3.51 | 21.10 | 230 | Red |
| | | 2-27 | | | | |
| Example 150 | | Compound | 3.53 | 21.06 | 215 | Red |
| | | 2-28 | | | | |
| Example 151 | Compound | Compound | 3.56 | 22.90 | 278 | Red |
| | 1-51 | 2-5 | | | | |
| Example 152 | | Compound | 3.61 | 21.87 | 290 | Red |
| | | 2-14 | | | | |
| Example 153 | | Compound | 3.65 | 21.08 | 264 | Red |
| | | 2-23 | | | | |
| Example 154 | | Compound | 3.67 | 21.01 | 215 | Red |
| | | 2-27 | | | | |
| Example 155 | | Compound | 3.52 | 22.16 | 278 | Red |
| | | 2-32 | | | | |
| Example 156 | Compound | Compound | 3.53 | 21.47 | 260 | Red |
| | 1-52 | 2-2 | | | | |
| Example 157 | | Compound | 3.60 | 21.15 | 278 | Red |
| | | 2-12 | | | | |
| Example 158 | | Compound | 3.52 | 21.13 | 295 | Red |
| | | 2-18 | | | | |
| Example 159 | | Compound | 3.51 | 21.18 | 261 | Red |
| | | 2-26 | | | | |
| Example 160 | | Compound | 3.53 | 21.08 | 232 | Red |
| | | 2-32 | | | | |

TABLE 5

| | | | Driving | | | |
|---|---|---|---|---|---|---|
| | First | Second | voltage | Efficiency | Lifetime | Luminous |
| Category | host | host | (V) | (cd/A) | T95(hr) | color |
| Example 161 | Compound | Compound | 3.45 | 23.23 | 345 | Red |
| | 1-53 | 2-9 | | | | |
| Example 162 | | Compound | 3.4 | 22.47 | 298 | Red |
| | | 2-10 | | | | |
| Example 163 | | Compound | 3.49 | 23.15 | 305 | Red |
| | | 2-11 | | | | |
| Example 164 | | Compound | 3.47 | 23.17 | 330 | Red |
| | | 2-30 | | | | |
| Example 165 | | Compound | 3.46 | 22.56 | 315 | Red |
| | | 2-31 | | | | |
| Example 166 | Compound | Compound | 3.49 | 22.13 | 356 | Red |
| | 1-55 | 2-9 | | | | |
| Example 167 | | Compound | 3.41 | 23.15 | 378 | Red |
| | | 2-10 | | | | |
| Example 168 | | Compound | 3.52 | 22.17 | 298 | Red |
| | | 2-11 | | | | |
| Example 169 | | Compound | 3.50 | 22.02 | 332 | Red |
| | | 2-30 | | | | |
| Example 170 | | Compound | 3.40 | 22.94 | 331 | Red |
| | | 2-31 | | | | |
| Example 171 | Compound | Compound | 3.43 | 22.45 | 350 | Red |
| | 1-57 | 2-4 | | | | |
| Example 172 | | Compound | 3.45 | 22.78 | 305 | Red |
| | | 2-13 | | | | |
| Example 173 | | Compound | 3.46 | 22.14 | 398 | Red |
| | | 2-21 | | | | |
| Example 174 | | Compound | 3.41 | 22.98 | 275 | Red |
| | | 2-28 | | | | |
| Example 175 | | Compound | 3.44 | 22.51 | 315 | Red |
| | | 2-31 | | | | |
| Example 176 | Compound | Compound | 3.58 | 21.54 | 298 | Red |
| | 1-58 | 2-7 | | | | |
| Example 177 | | Compound | 3.59 | 22.13 | 281 | Red |
| | | 2-15 | | | | |
| Example 178 | | Compound | 3.61 | 22.36 | 275 | Red |
| | | 2-20 | | | | |
| Example 179 | | Compound | 3.68 | 22.19 | 296 | Red |
| | | 2-26 | | | | |
| Example 180 | | Compound | 3.72 | 22.50 | 287 | Red |
| | | 2-29 | | | | |
| Example 181 | Compound | Compound | 3.43 | 23.15 | 339 | Red |
| | 1-59 | 2-8 | | | | |
| Example 182 | | Compound | 3.41 | 22.12 | 296 | Red |
| | | 2-10 | | | | |
| Example 183 | | Compound | 3.42 | 22.15 | 303 | Red |
| | | 2-22 | | | | |
| Example 184 | | Compound | 3.43 | 22.80 | 300 | Red |
| | | 2-27 | | | | |
| Example 185 | | Compound | 3.4 | 22.09 | 310 | Red |
| | | 2-33 | | | | |
| Example 186 | Compound | Compound | 3.53 | 21.03 | 298 | Red |
| | 1-61 | 2-6 | | | | |
| Example 187 | | Compound | 3.54 | 21.15 | 230 | Red |
| | | 2-16 | | | | |
| Example 188 | | Compound | 3.59 | 21.48 | 278 | Red |
| | | 2-24 | | | | |
| Example 189 | | Compound | 3.55 | 21.03 | 267 | Red |
| | | 2-25 | | | | |
| Example 190 | | Compound | 3.54 | 21.15 | 225 | Red |
| | | 2-34 | | | | |

TABLE 6

| | | | Driving | | | |
|---|---|---|---|---|---|---|
| | First | Second | voltage | Efficiency | Lifetime | Luminous |
| Category | host | host | (V) | (cd/A) | T95(hr) | color |
| Comparative | Compound | Compound | 3.99 | 18.15 | 140 | Red |
| Example 1 | 1-6 | B-1 | | | | |
| Comparative | | Compound | 3.96 | 17.12 | 172 | Red |
| Example 2 | | B-2 | | | | |
| Comparative | | Compound | 3.87 | 17.13 | 186 | Red |
| Example 3 | | B-3 | | | | |
| Comparative | | Compound | 3.91 | 17.52 | 141 | Red |
| Example 4 | | B-4 | | | | |
| Comparative | Compound | Compound | 3.82 | 17.64 | 152 | Red |
| Example 5 | 1-9 | B-1 | | | | |
| Comparative | | Compound | 3.93 | 17.47 | 186 | Red |
| Example 6 | | B-2 | | | | |
| Comparative | | Compound | 3.86 | 18.51 | 171 | Red |
| Example 7 | | B-3 | | | | |
| Comparative | | Compound | 3.99 | 17.59 | 173 | Red |
| Example 8 | | B-4 | | | | |
| Comparative | Compound | Compound | 4.14 | 16.16 | 100 | Red |
| Example 9 | 1-19 | B-1 | | | | |
| Comparative | | Compound | 4.11 | 15.97 | 121 | Red |
| Example 10 | | B-2 | | | | |
| Comparative | | Compound | 4.21 | 14.06 | 112 | Red |
| Example 11 | | B-3 | | | | |
| Comparative | | Compound | 4.18 | 14.48 | 115 | Red |
| Example 12 | | B-4 | | | | |

As shown in Tables 1 to 6, the organic light emitting devices of Examples, in which the first compound represented by Chemical Formula 1 and the second compound represented by Chemical Formula 2 were simultaneously used as the host materials of the light emitting layer, exhibited excellent driving voltage, luminous efficiency and lifetime characteristics as compared to the organic light emitting devices of Comparative Examples, in which a compound having a structure different from one of the compounds represented by Chemical Formulas 1 and 2 was used, or only a compound having a different structure was used.

In particular, the devices according to Examples has all improved driving voltage, efficiency, and lifetime characteristics as compared to all the devices of Comparative Examples in which the compound represented by Chemical Formula 1 was employed as the first host and the comparative compounds B-1 to B-4 was employed as the second host. Through this, it was confirmed that when the combination of the first compound represented by Chemical Formula 1 and the second compound represented by Chemical Formula 2 was used as a cohost, energy transfer to the red dopant in the red light emitting layer was effectively performed. This is believed to be because the cohost combination of Examples maintained a more stable balance in the light emitting layer than the cohost combination of the devices of Comparative Examples.

Therefore, it could be confirmed that when the first compound and the second compound are simultaneously employed as host materials for an organic light emitting device, the driving voltage, luminous efficiency, and lifetime characteristics of the organic light emitting devices can be improved. Considering that the luminous efficiency and lifetime characteristics of the organic light emitting devices generally have a trade-off relationship, the organic light emitting devices employing the combination of the compounds of the present disclosure exhibit significantly improved characteristics as compared to the devices of Comparative Examples.

[Description of Symbols]

| | |
|---|---|
| 1: substrate | 2: anode |
| 3: light emitting layer | 4: cathode |
| 5: hole injection layer | 6: hole transport layer |
| 7: electron blocking layer | 8: hole blocking layer |
| 9: electron injection and transport layer | |

1. An organic light emitting device comprising:
an anode;
a cathode which is provided opposite to the anode; and
a light emitting layer which is interposed between the anode and the cathode,
wherein the light emitting layer includes a first compound represented by the following Chemical Formula 1, and a second compound represented by the following Chemical Formula 2: 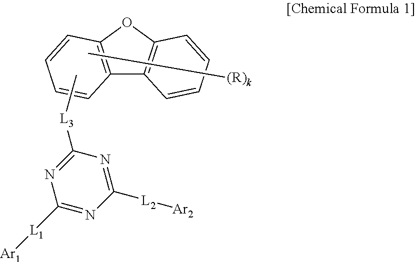
wherein in Chemical Formula 1:
L₁ to L₃ are each independently a single bond or a substituted or unsubstituted C₆₋₆₀ arylene;
Ar₁ and Ar₂ are each independently a substituted or unsubstituted C₆₋₆₀ aryl or a substituted or unsubstituted C₂₋₆₀ heteroaryl containing at least one heteroatom selected among N, O and S;
R is hydrogen, deuterium, halogen, cyano, or a substituted or unsubstituted C₁₋₆₀ alkyl;
k is an integer of 0 to 7; 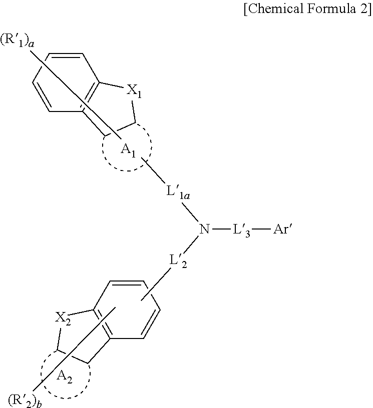
wherein in Chemical Formula 2:
X₁ and X₂ are each independently O or S;
A₁ and A₂ are each independently a naphthalene ring fused with an adjacent pentagonal ring;
L′₁ to L′₃ are each independently a single bond or a substituted or unsubstituted C₆₋₆₀ arylene;
Ar′ is a substituted or unsubstituted C₆₋₆₀ aryl, or a substituted or unsubstituted C₂₋₆₀ heteroaryl containing at least one heteroatom selected among N, O and S;
R′₁ and R′₂ are each independently hydrogen, deuterium, halogen, cyano, or a substituted or unsubstituted C₁₋₆₀ alkyl; and
a and b are each independently an integer of 0 to 9.

2. The organic light emitting device of claim 1, wherein:
the Chemical Formula 1 is represented by the following Chemical Formula 1-1: 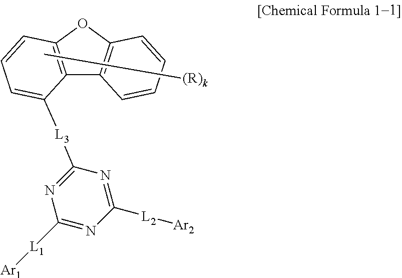
wherein in Chemical Formula 1-1:
L₁ to L₃, Ar₁, Ar₂, R and k are as defined in claim 1.

3. The organic light emitting device of claim 1, wherein:
L₁ and L₂ are each independently a single bond, or a substituted or unsubstituted C₆₋₁₂ arylene.

4. The organic light emitting device of claim 1, wherein:
L₃ is a single bond, a substituted or unsubstituted phenylene, or a substituted or unsubstituted naphthylene.

5. The organic light emitting device of claim 1, wherein:
Ar₁ and Ar₂ are each independently phenyl, biphenylyl, terphenylyl, naphthyl, phenanthryl, dibenzofuranyl, dibenzothiophenyl, benzonaphthofuranyl, or benzonaphthothiophenyl,
wherein the Ar₁ and Ar₂ are unsubstituted, or substituted with one or more substituents selected from the group consisting of deuterium, halogen, cyano, phenyl substituted or unsubstituted with deuterium, biphenylyl substituted or unsubstituted with deuterium, naphthyl substituted or unsubstituted with deuterium, and —Si(phenyl substituted or unsubstituted with deuterium)₃.

6. The organic light emitting device of claim 1, wherein:
k is 0; or
k is 1 or more, and R is deuterium.

7. The organic light emitting device of claim 1, wherein:
the first compound is any one compound selected from among the following compounds:

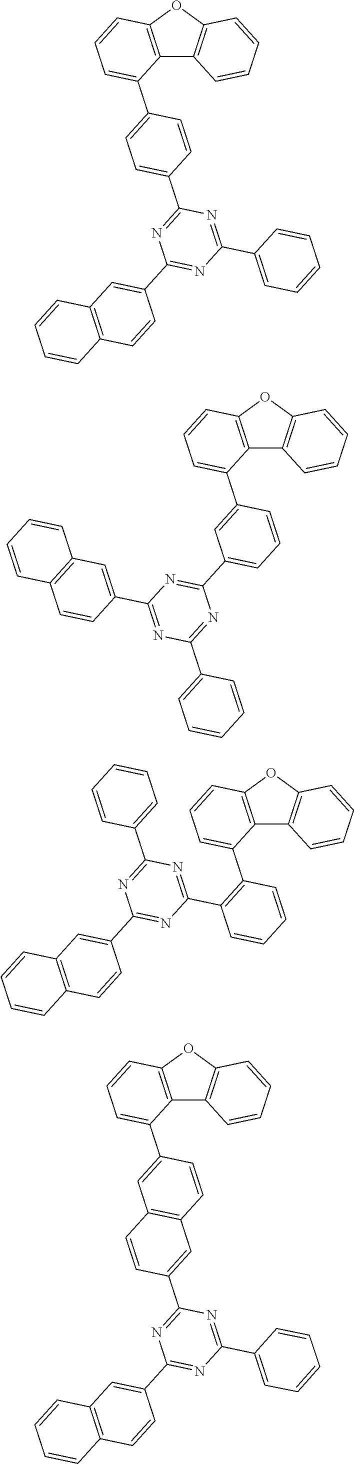 [SMILES: c1ccc(-c2nc(-c3ccc(-c4cccc5oc6ccccc6c45)cc3)nc(-c3ccc4ccccc4c3)n2)cc1 ; c1ccc(-c2nc(-c3cccc(-c4cccc5oc6ccccc6c45)c3)nc(-c3ccc4ccccc4c3)n2)cc1 ; c1ccc(-c2nc(-c3ccc4ccccc4c3)nc(-c3ccccc3-c3cccc4oc5ccccc5c34)n2)cc1 ; c1ccc(-c2nc(-c3ccc4ccccc4c3)nc(-c3ccc4cc(-c5cccc6oc7ccccc7c56)ccc4c3)n2)cc1]

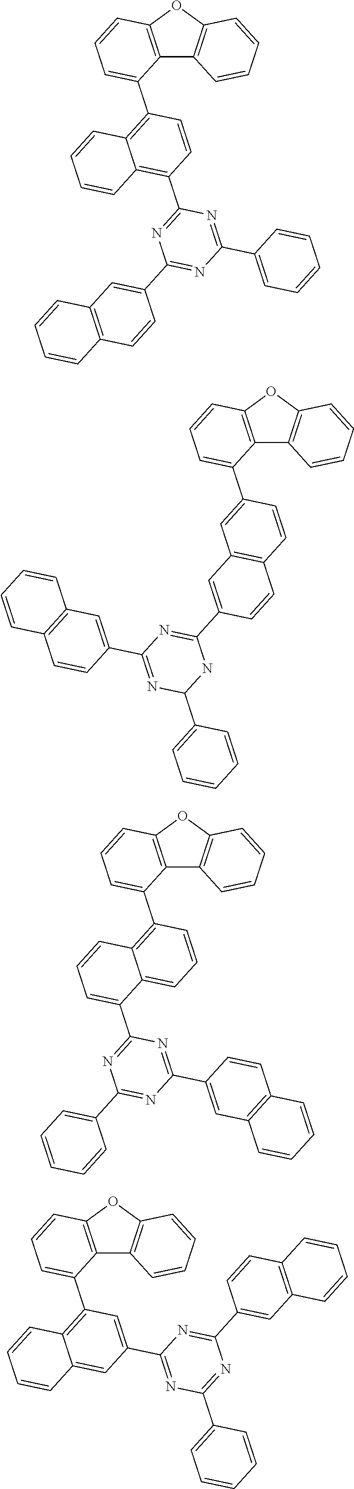

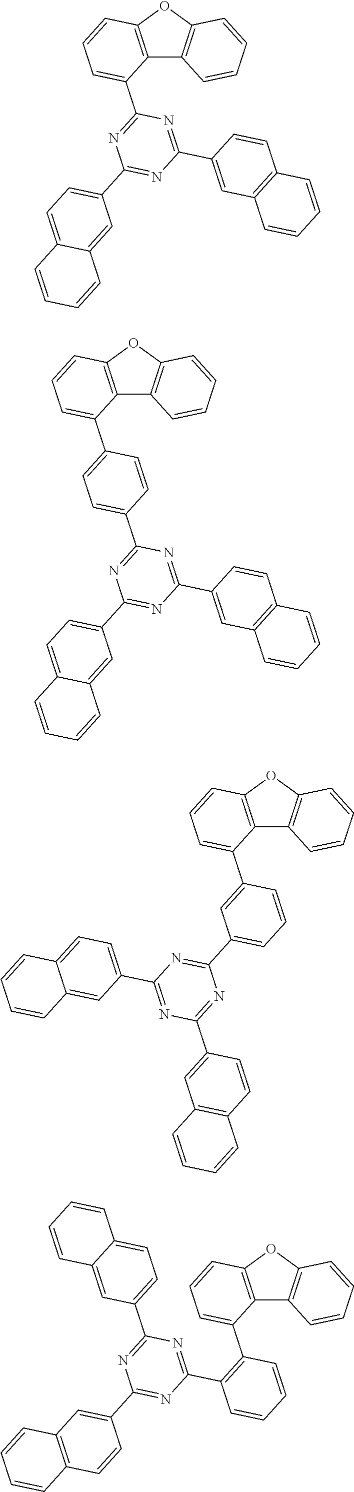 [SMILES: c1ccc2cc(-c3nc(-c4ccc5ccccc5c4)nc(-c4cccc5oc6ccccc6c45)n3)ccc2c1 ; c1ccc2cc(-c3nc(-c4ccc(-c5cccc6oc7ccccc7c56)cc4)nc(-c4ccc5ccccc5c4)n3)ccc2c1 ; c1cc(-c2nc(-c3ccc4ccccc4c3)nc(-c3ccc4ccccc4c3)n2)cc(-c2cccc3oc4ccccc4c23)c1 ; c1ccc(-c2cccc3oc4ccccc4c23)c(-c2nc(-c3ccc4ccccc4c3)nc(-c3ccc4ccccc4c3)n2)c1]

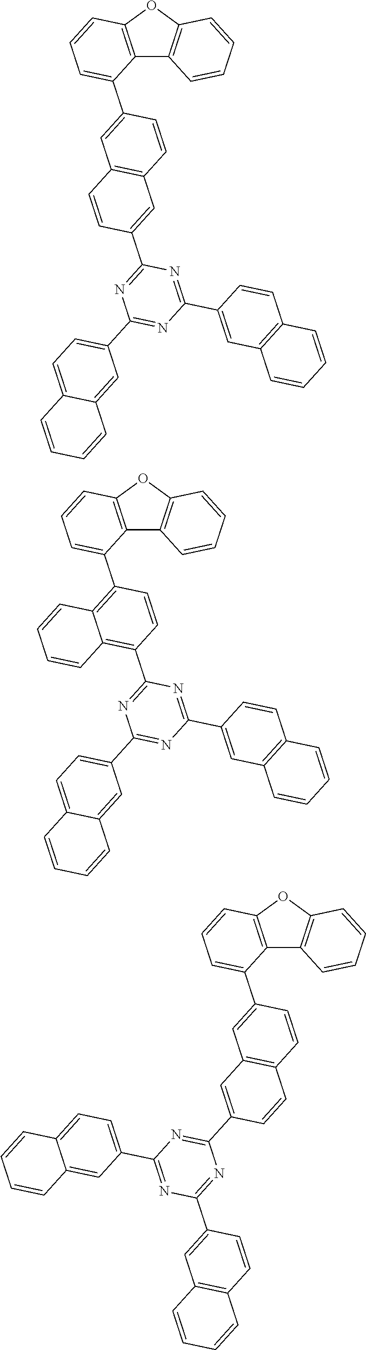

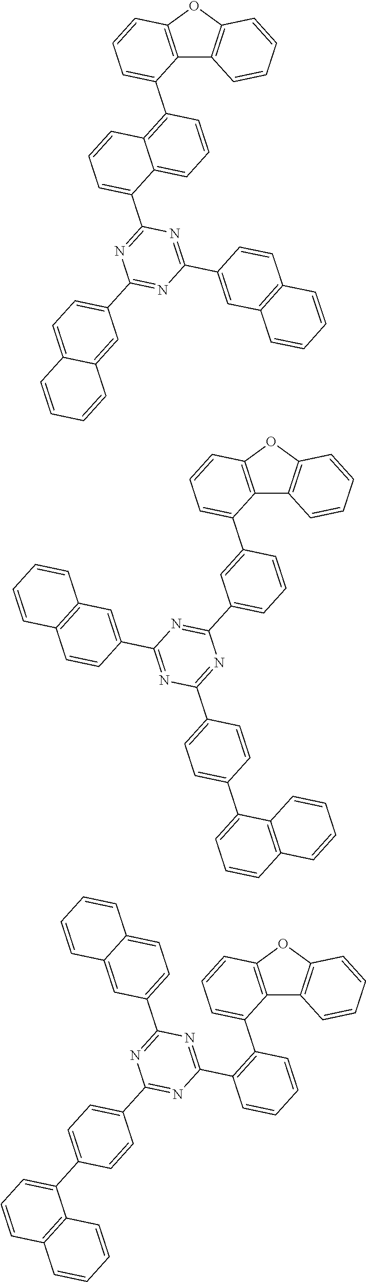 [SMILES: c1ccc2cc(-c3nc(-c4ccc5ccccc5c4)nc(-c4cccc5c(-c6cccc7oc8ccccc8c67)cccc45)n3)ccc2c1 ; c1cc(-c2nc(-c3ccc(-c4cccc5ccccc45)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4cccc5ccccc45)cc3)nc(-c3ccc4ccccc4c3)n2)c1]

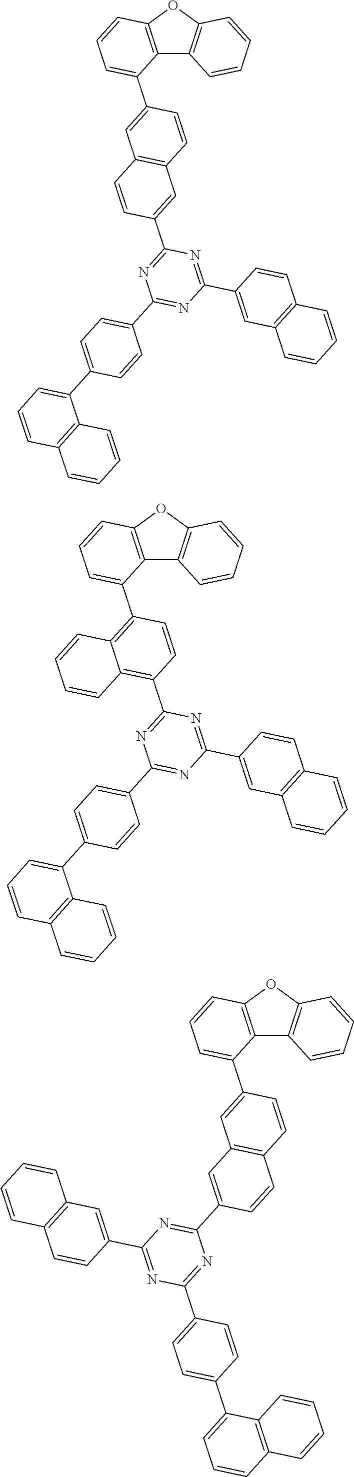

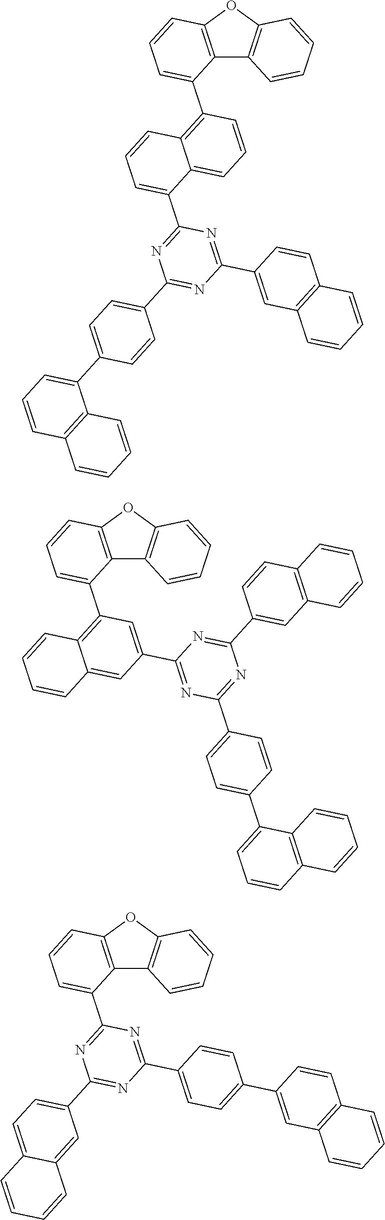

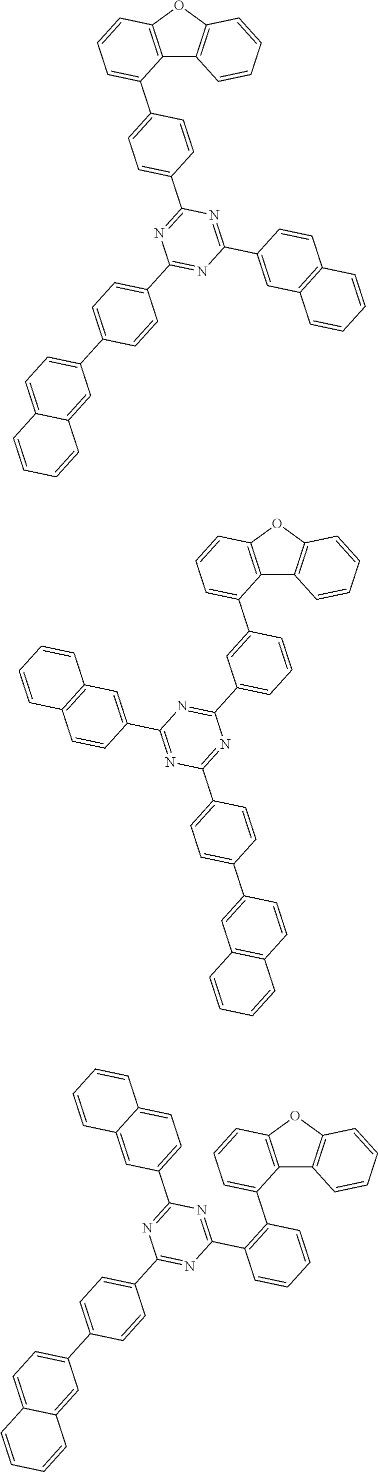 [SMILES: c1ccc2cc(-c3ccc(-c4nc(-c5ccc(-c6cccc7oc8ccccc8c67)cc5)nc(-c5ccc6ccccc6c5)n4)cc3)ccc2c1 ; c1cc(-c2nc(-c3ccc(-c4ccc5ccccc5c4)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4ccc5ccccc5c4)cc3)nc(-c3ccc4ccccc4c3)n2)c1]

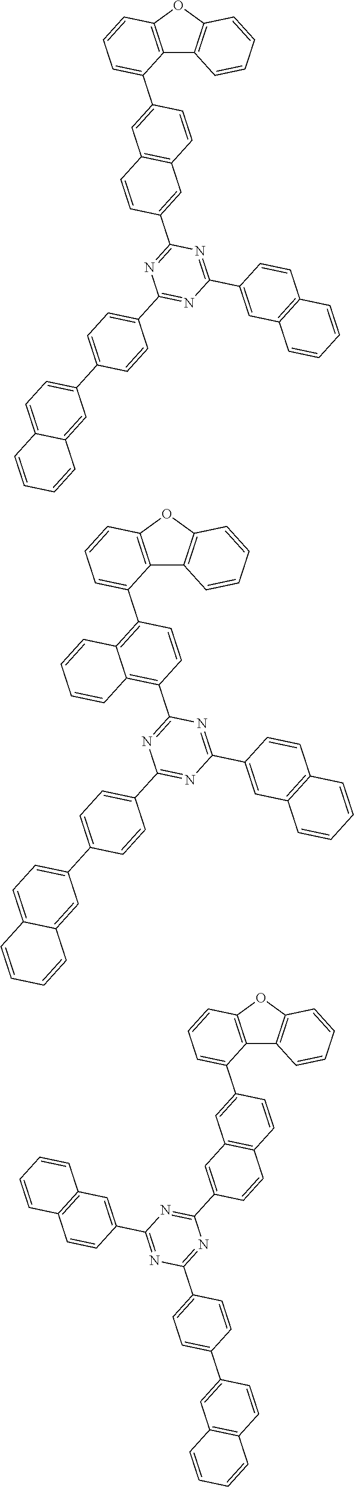 [SMILES: c1ccc2cc(-c3ccc(-c4nc(-c5ccc6ccccc6c5)nc(-c5ccc6cc(-c7cccc8oc9ccccc9c78)ccc6c5)n4)cc3)ccc2c1 ; c1ccc2cc(-c3ccc(-c4nc(-c5ccc6ccccc6c5)nc(-c5ccc(-c6cccc7oc8ccccc8c67)c6ccccc56)n4)cc3)ccc2c1 ; c1ccc2cc(-c3ccc(-c4nc(-c5ccc6ccccc6c5)nc(-c5ccc6ccc(-c7cccc8oc9ccccc9c78)cc6c5)n4)cc3)ccc2c1]

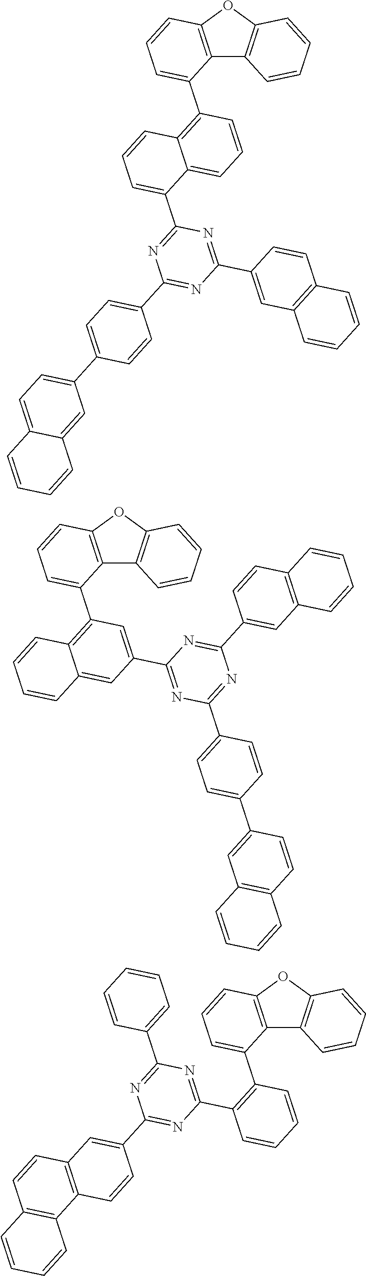

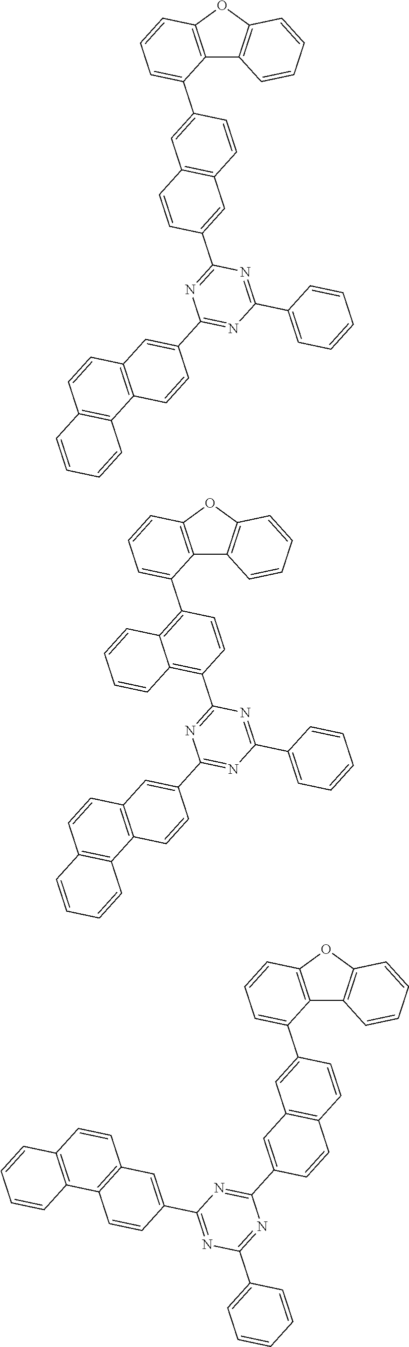 [SMILES: c1ccc(-c2nc(-c3ccc4cc(-c5cccc6oc7ccccc7c56)ccc4c3)nc(-c3ccc4c(ccc5ccccc54)c3)n2)cc1 ; c1ccc(-c2nc(-c3ccc4c(ccc5ccccc54)c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(ccc5ccccc54)c3)n2)cc1]

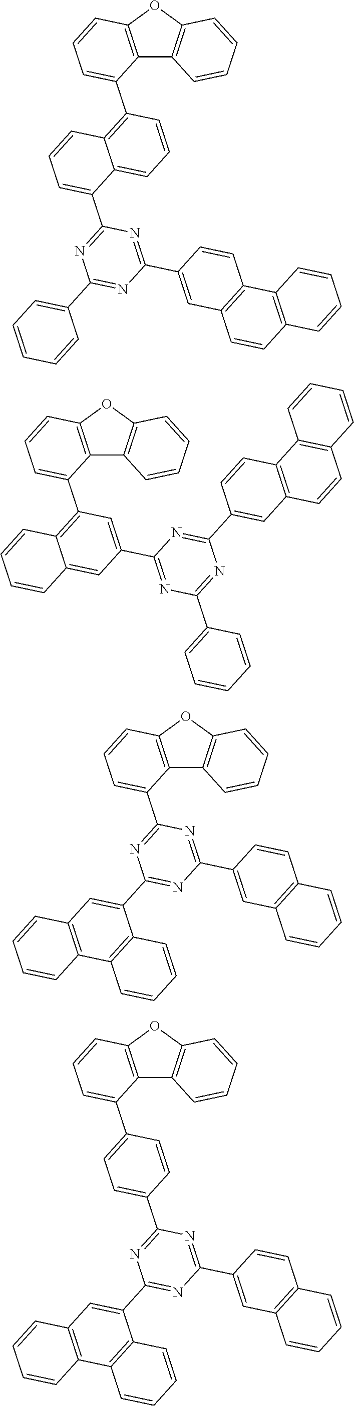

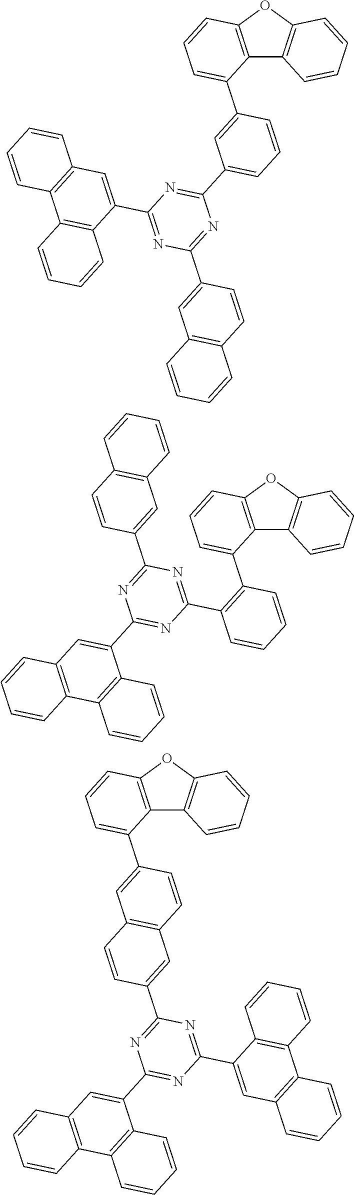

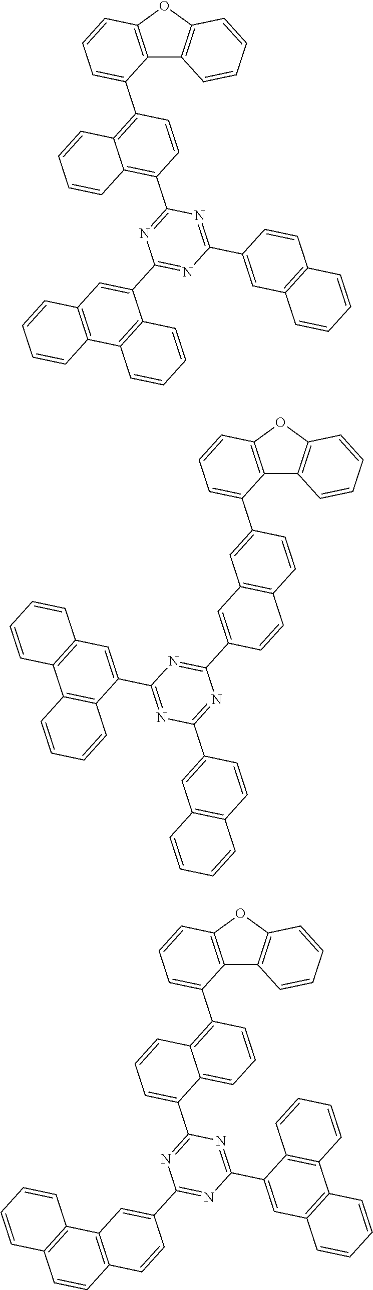 [SMILES: c1ccc2cc(-c3nc(-c4ccc(-c5cccc6oc7ccccc7c56)c5ccccc45)nc(-c4cc5ccccc5c5ccccc45)n3)ccc2c1 ; c1ccc2cc(-c3nc(-c4ccc5ccc(-c6cccc7oc8ccccc8c67)cc5c4)nc(-c4cc5ccccc5c5ccccc45)n3)ccc2c1 ; c1ccc2c(c1)ccc1ccc(-c3nc(-c4cccc5c(-c6cccc7oc8ccccc8c67)cccc45)nc(-c4cc5ccccc5c5ccccc45)n3)cc12]

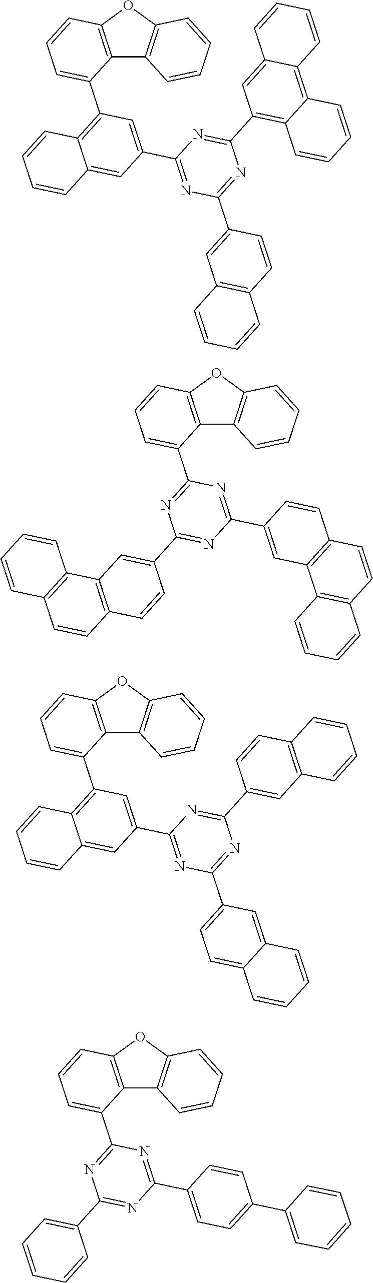

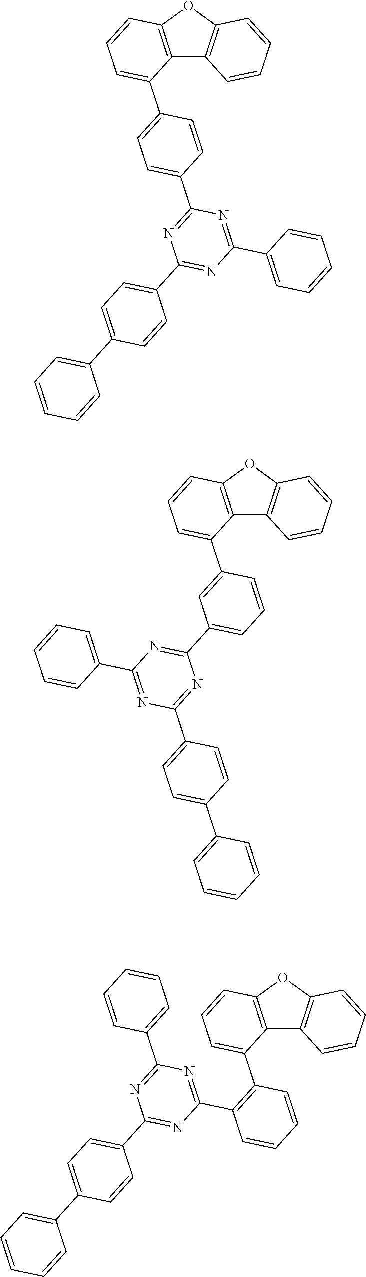 [SMILES: c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4cccc(-c5cccc6oc7ccccc7c56)c4)n3)cc2)cc1 ; c1ccc(-c2ccc(-c3nc(-c4ccccc4)nc(-c4ccccc4-c4cccc5oc6ccccc6c45)n3)cc2)cc1]

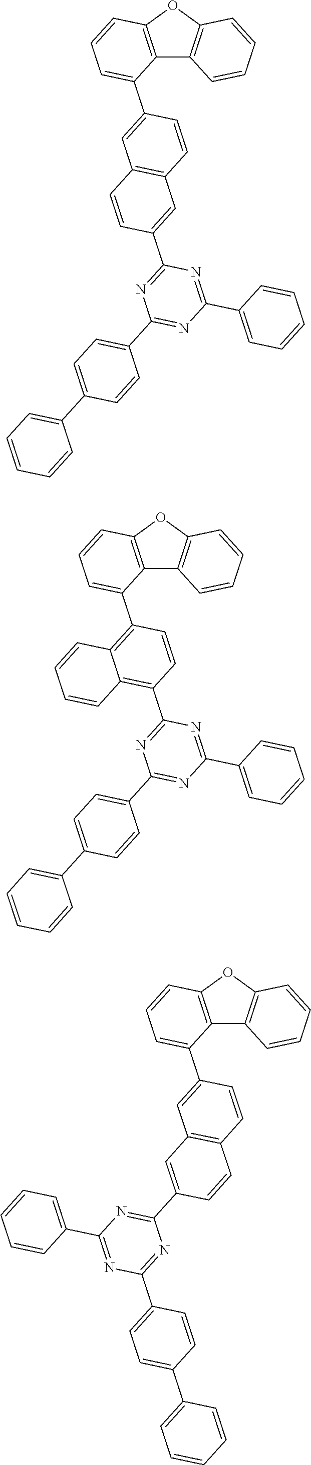 [SMILES: c1ccc(-c2ccc(-c3nc(-c4ccccc4)nc(-c4ccc5cc(-c6cccc7oc8ccccc8c67)ccc5c4)n3)cc2)cc1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2ccc(-c3nc(-c4ccccc4)nc(-c4ccc5ccc(-c6cccc7oc8ccccc8c67)cc5c4)n3)cc2)cc1]

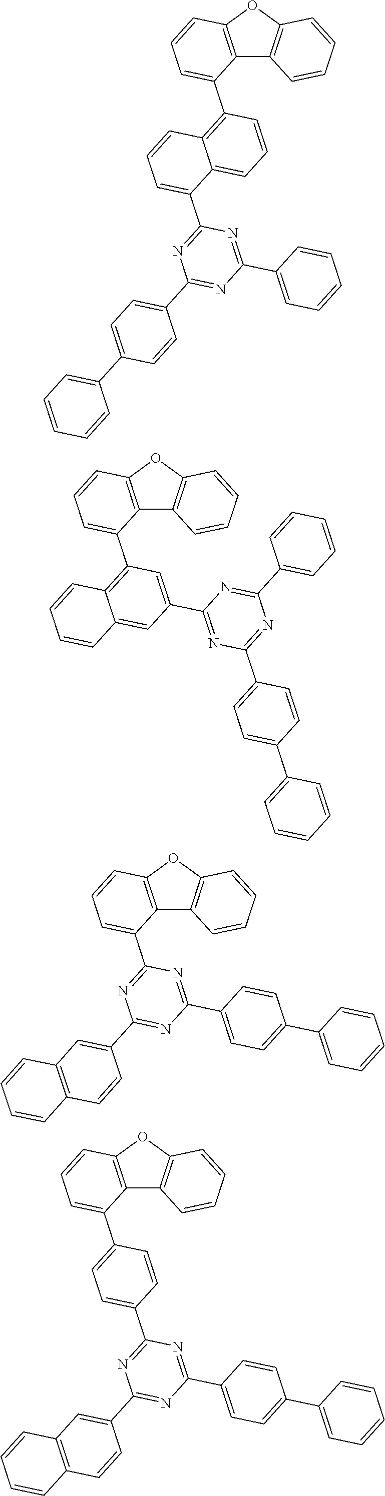

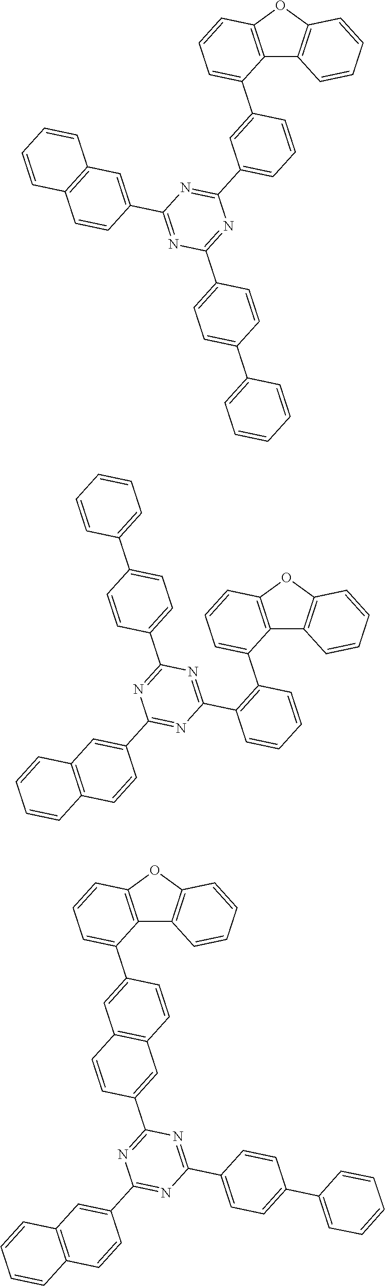 [SMILES: c1ccc(-c2ccc(-c3nc(-c4cccc(-c5cccc6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6ccccc6c45)n3)cc2)cc1 ; c1ccc(-c2ccc(-c3nc(-c4ccc5ccccc5c4)nc(-c4ccc5cc(-c6cccc7oc8ccccc8c67)ccc5c4)n3)cc2)cc1]

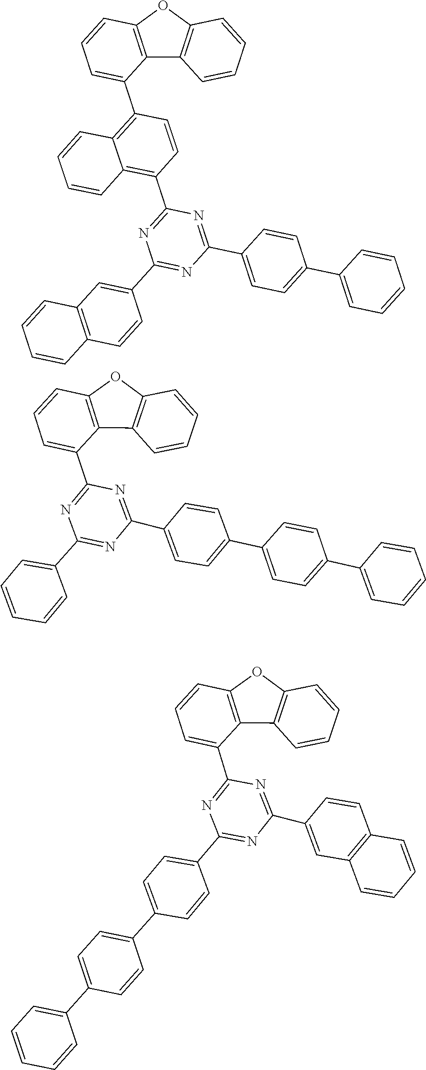

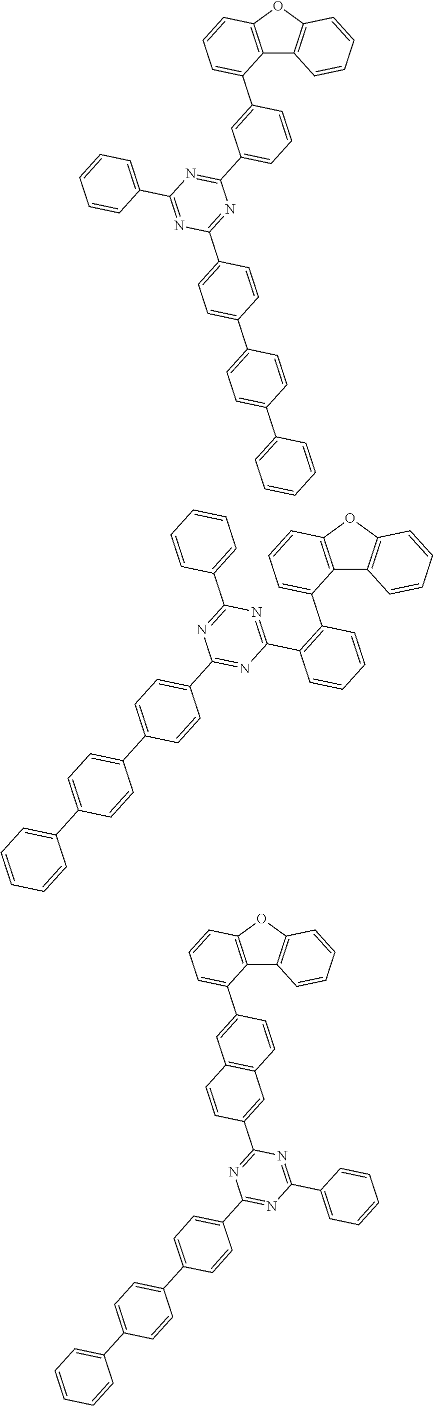

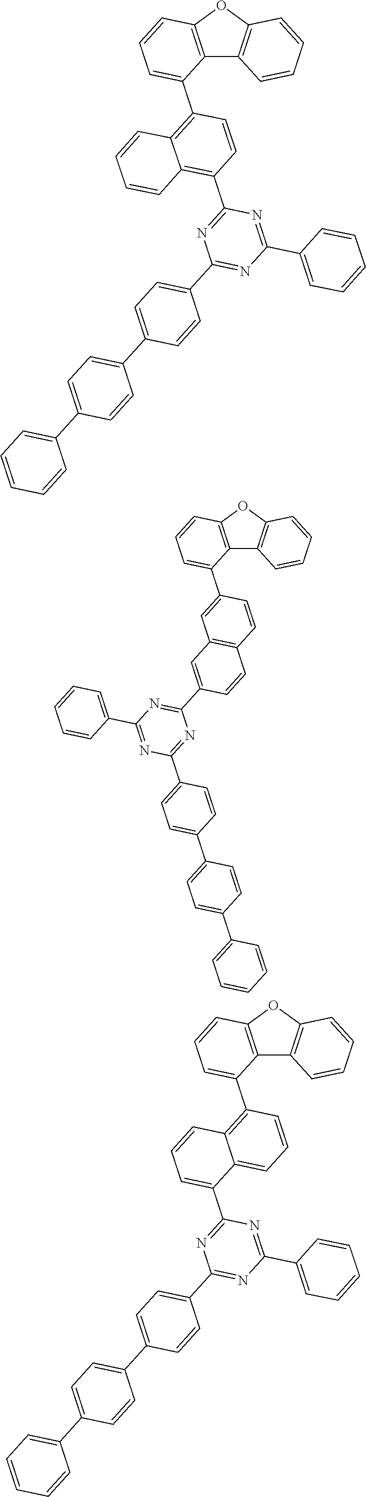

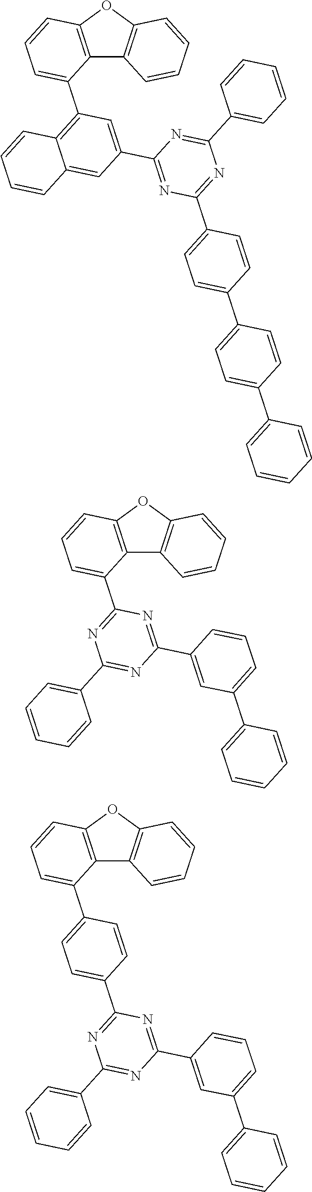 [SMILES: c1ccc(-c2ccc(-c3ccc(-c4nc(-c5ccccc5)nc(-c5cc(-c6cccc7oc8ccccc8c67)c6ccccc6c5)n4)cc3)cc2)cc1 ; c1ccc(-c2cccc(-c3nc(-c4ccccc4)nc(-c4cccc5oc6ccccc6c45)n3)c2)cc1 ; c1ccc(-c2cccc(-c3nc(-c4ccccc4)nc(-c4ccc(-c5cccc6oc7ccccc7c56)cc4)n3)c2)cc1]

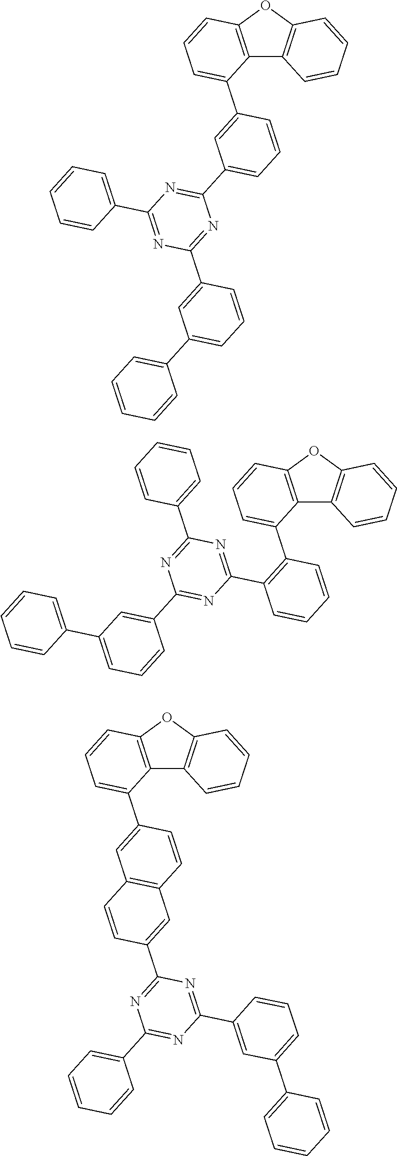

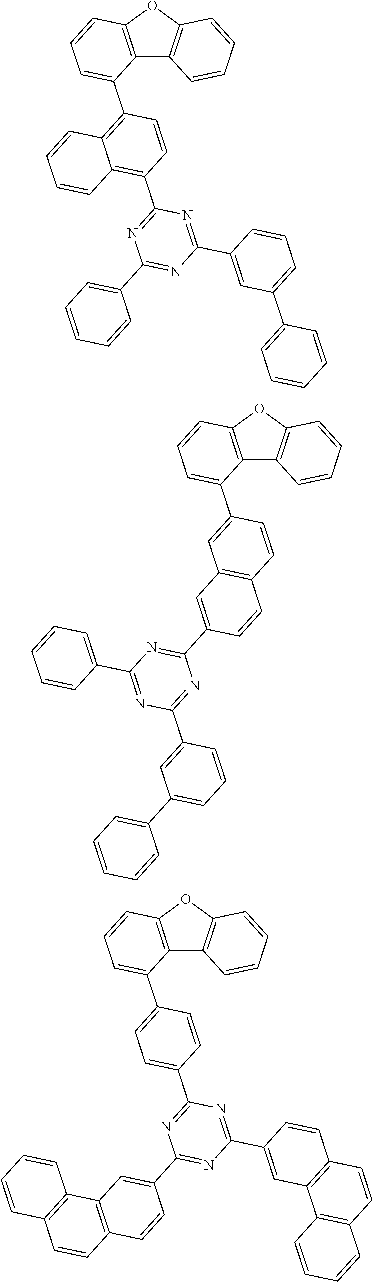

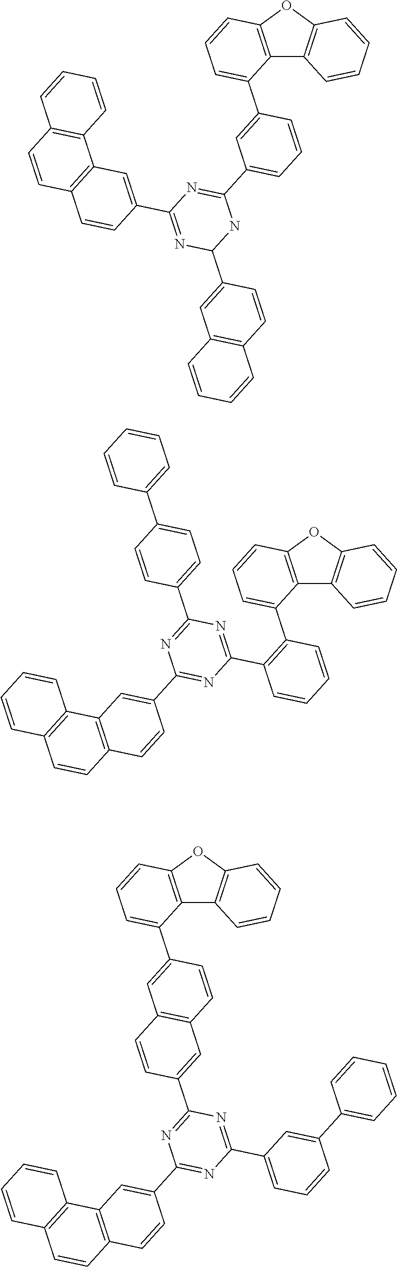

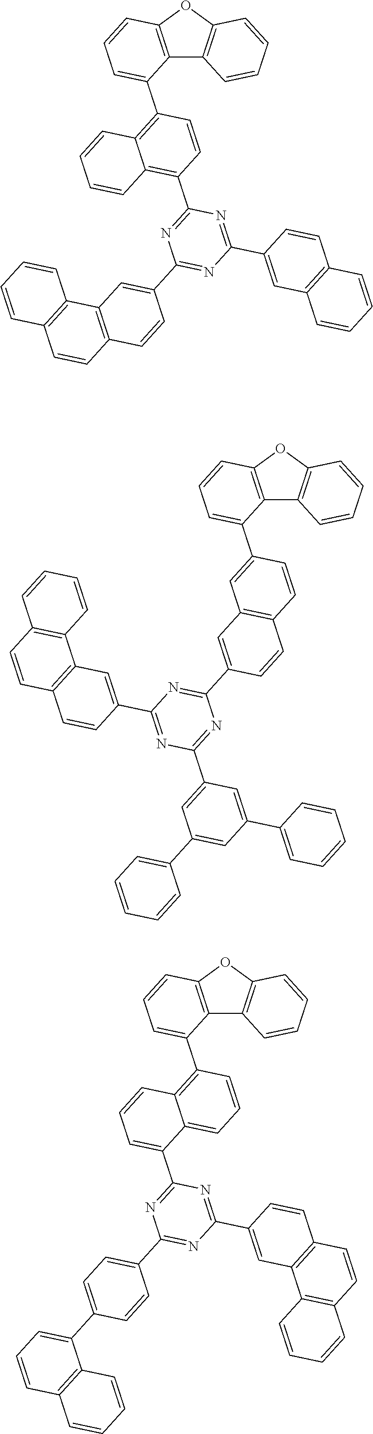 [SMILES: c1ccc2cc(-c3nc(-c4ccc5ccc6ccccc6c5c4)nc(-c4ccc(-c5cccc6oc7ccccc7c56)c5ccccc45)n3)ccc2c1 ; c1ccc(-c2cc(-c3ccccc3)cc(-c3nc(-c4ccc5ccc(-c6cccc7oc8ccccc8c67)cc5c4)nc(-c4ccc5ccc6ccccc6c5c4)n3)c2)cc1 ; c1ccc2c(-c3ccc(-c4nc(-c5ccc6ccc7ccccc7c6c5)nc(-c5cccc6c(-c7cccc8oc9ccccc9c78)cccc56)n4)cc3)cccc2c1]

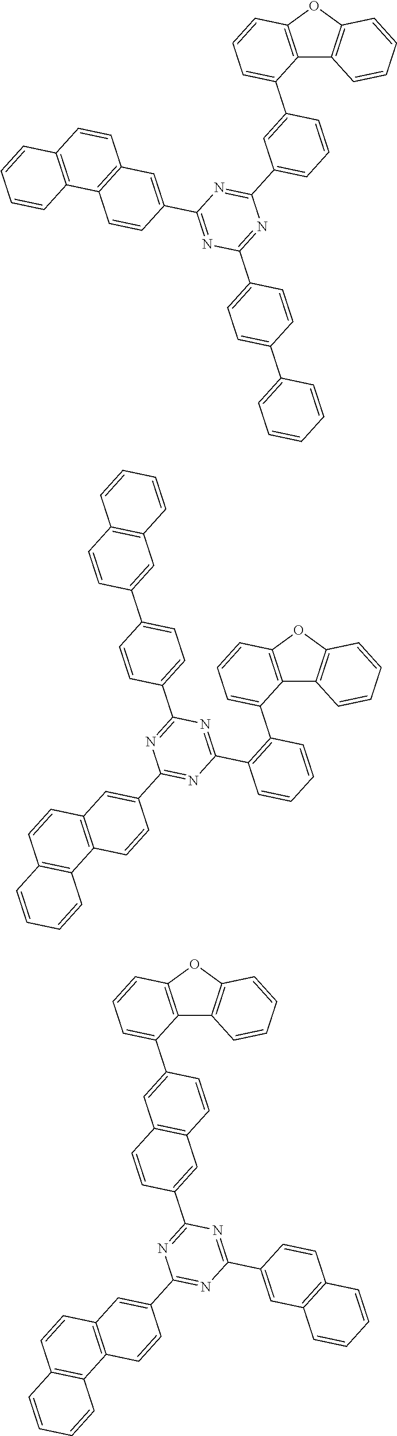 [SMILES: c1ccc(-c2ccc(-c3nc(-c4cccc(-c5cccc6oc7ccccc7c56)c4)nc(-c4ccc5c(ccc6ccccc65)c4)n3)cc2)cc1 ; c1ccc(-c2cccc3oc4ccccc4c23)c(-c2nc(-c3ccc(-c4ccc5ccccc5c4)cc3)nc(-c3ccc4c(ccc5ccccc54)c3)n2)c1 ; c1ccc2cc(-c3nc(-c4ccc5cc(-c6cccc7oc8ccccc8c67)ccc5c4)nc(-c4ccc5c(ccc6ccccc65)c4)n3)ccc2c1]

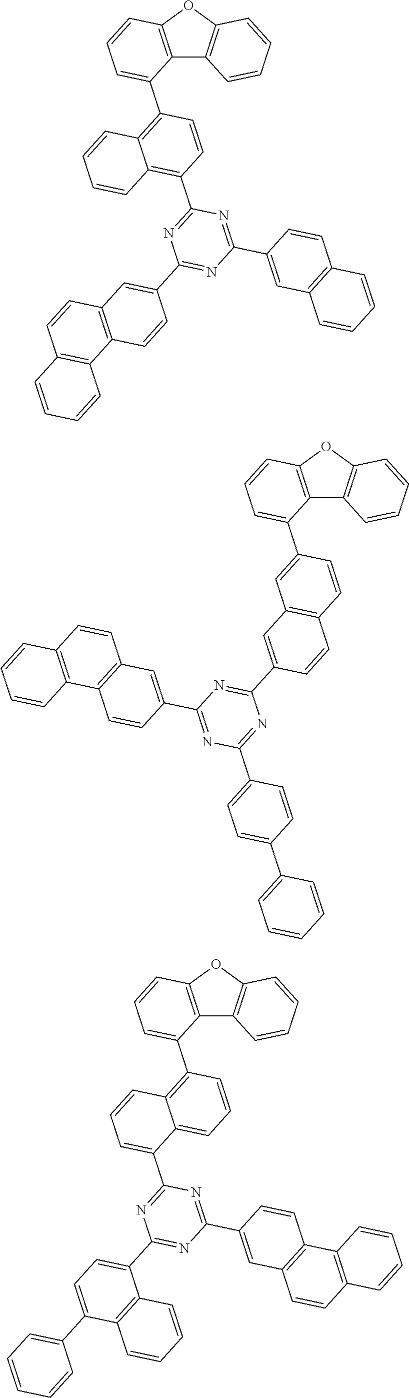

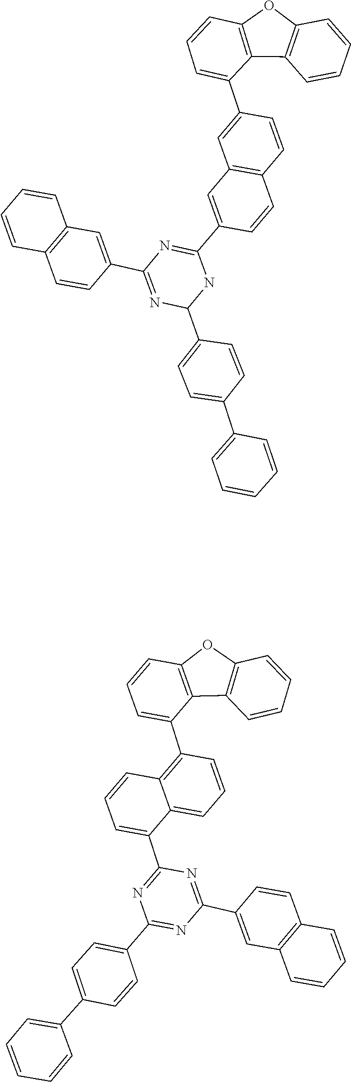

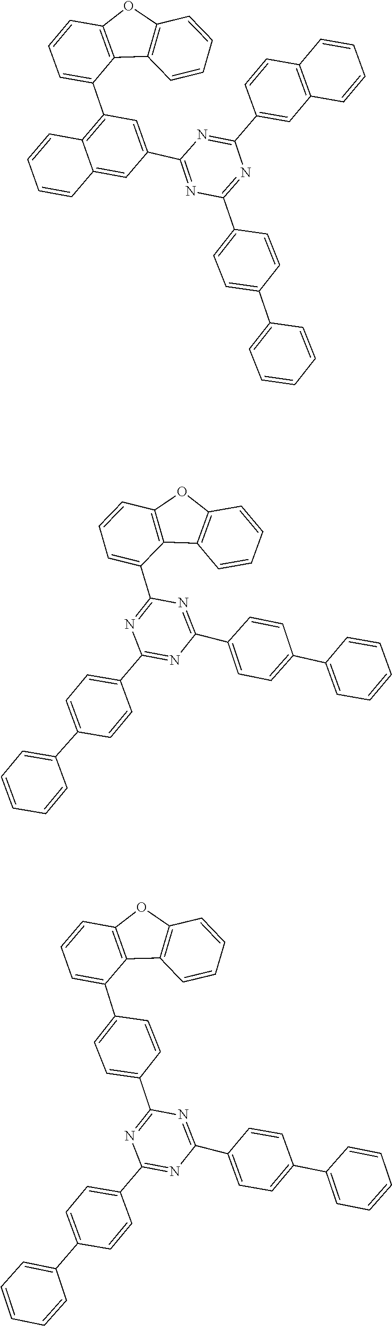 [SMILES: c1ccc(-c2ccc(-c3nc(-c4ccc5ccccc5c4)nc(-c4cc(-c5cccc6oc7ccccc7c56)c5ccccc5c4)n3)cc2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4ccc(-c5ccccc5)cc4)nc(-c4ccc(-c5cccc6oc7ccccc7c56)cc4)n3)cc2)cc1]

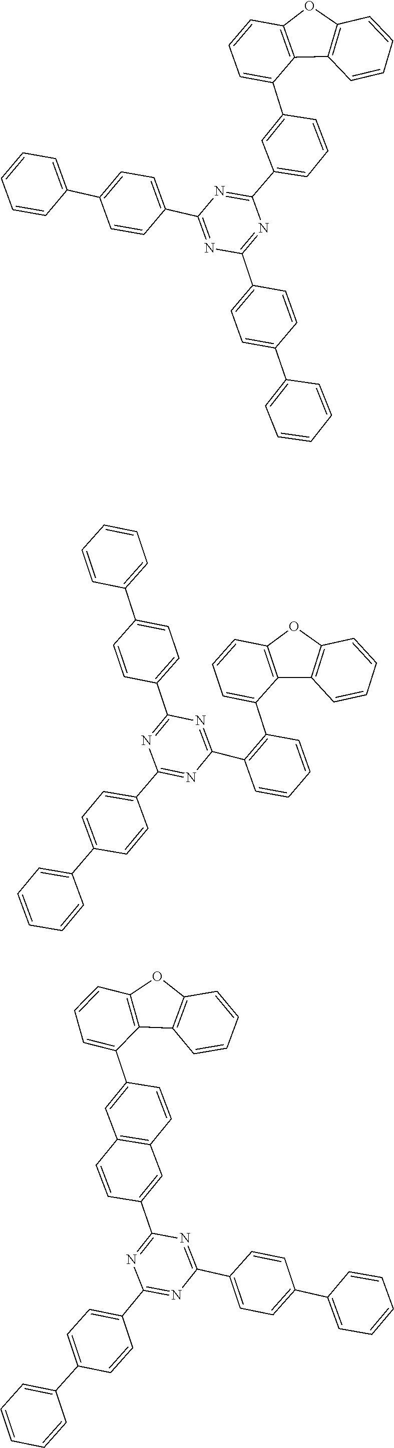 [SMILES: c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2ccc(-c3nc(-c4ccc(-c5ccccc5)cc4)nc(-c4ccccc4-c4cccc5oc6ccccc6c45)n3)cc2)cc1 ; c1ccc(-c2ccc(-c3nc(-c4ccc(-c5ccccc5)cc4)nc(-c4ccc5cc(-c6cccc7oc8ccccc8c67)ccc5c4)n3)cc2)cc1]

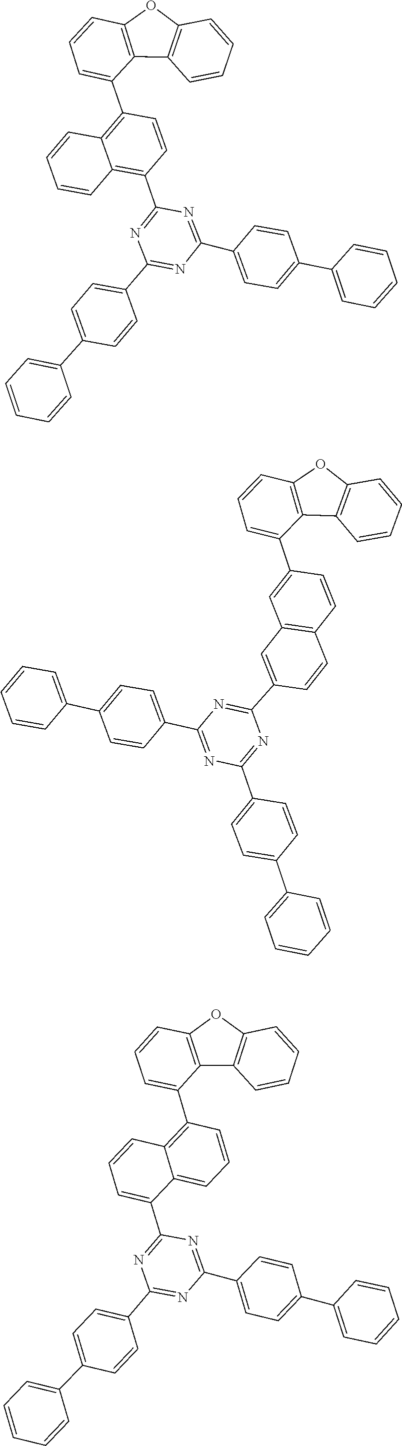 [SMILES: c1ccc(-c2ccc(-c3nc(-c4ccc(-c5ccccc5)cc4)nc(-c4ccc(-c5cccc6oc7ccccc7c56)c5ccccc45)n3)cc2)cc1 ; c1ccc(-c2ccc(-c3nc(-c4ccc(-c5ccccc5)cc4)nc(-c4ccc5ccc(-c6cccc7oc8ccccc8c67)cc5c4)n3)cc2)cc1 ; c1ccc(-c2ccc(-c3nc(-c4ccc(-c5ccccc5)cc4)nc(-c4cccc5c(-c6cccc7oc8ccccc8c67)cccc45)n3)cc2)cc1]

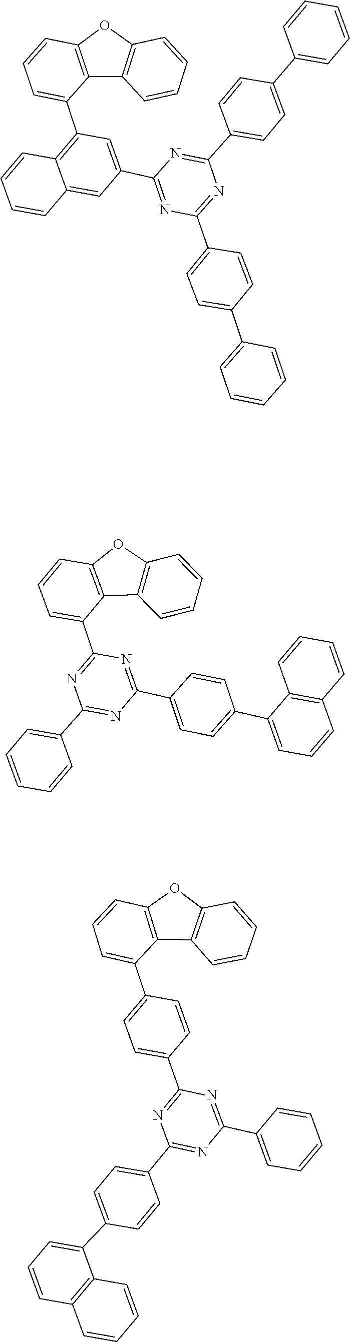 [SMILES: c1ccc(-c2ccc(-c3nc(-c4ccc(-c5ccccc5)cc4)nc(-c4cc(-c5cccc6oc7ccccc7c56)c5ccccc5c4)n3)cc2)cc1 ; c1ccc(-c2nc(-c3ccc(-c4cccc5ccccc45)cc3)nc(-c3cccc4oc5ccccc5c34)n2)cc1 ; c1ccc(-c2nc(-c3ccc(-c4cccc5ccccc45)cc3)nc(-c3ccc(-c4cccc5oc6ccccc6c45)cc3)n2)cc1]

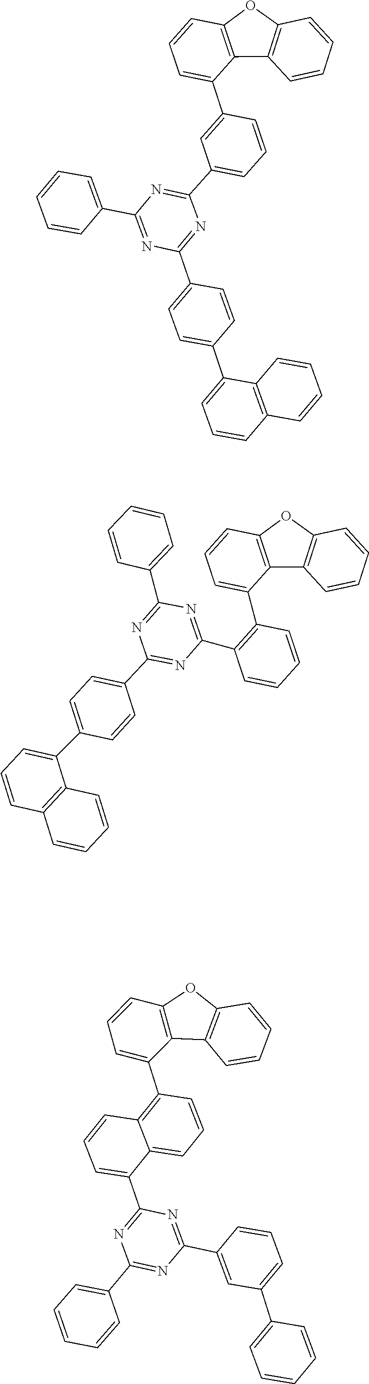 [SMILES: c1ccc(-c2nc(-c3ccc(-c4cccc5ccccc45)cc3)nc(-c3cccc(-c4cccc5oc6ccccc6c45)c3)n2)cc1 ; c1ccc(-c2nc(-c3ccc(-c4cccc5ccccc45)cc3)nc(-c3ccccc3-c3cccc4oc5ccccc5c34)n2)cc1 ; c1ccc(-c2cccc(-c3nc(-c4ccccc4)nc(-c4cccc5c(-c6cccc7oc8ccccc8c67)cccc45)n3)c2)cc1]

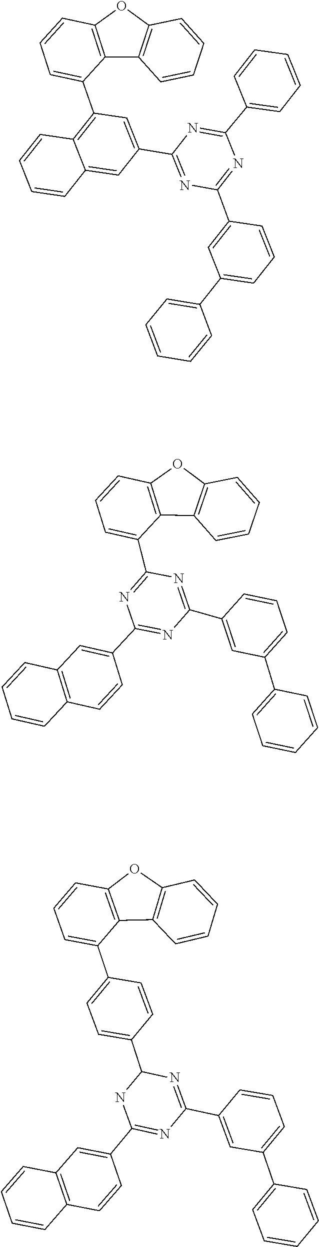

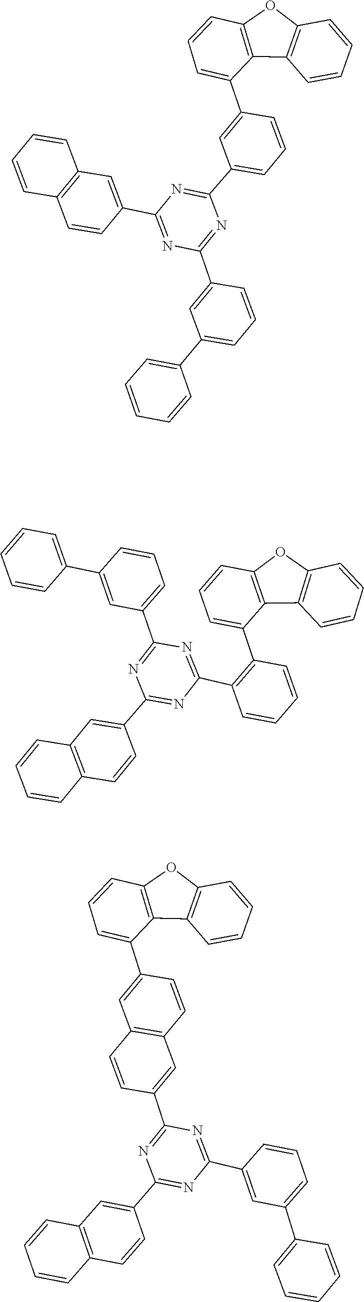 [SMILES: c1ccc(-c2cccc(-c3nc(-c4cccc(-c5cccc6oc7ccccc7c56)c4)nc(-c4ccc5ccccc5c4)n3)c2)cc1 ; c1ccc(-c2cccc(-c3nc(-c4ccc5ccccc5c4)nc(-c4ccccc4-c4cccc5oc6ccccc6c45)n3)c2)cc1 ; c1ccc(-c2cccc(-c3nc(-c4ccc5ccccc5c4)nc(-c4ccc5cc(-c6cccc7oc8ccccc8c67)ccc5c4)n3)c2)cc1]

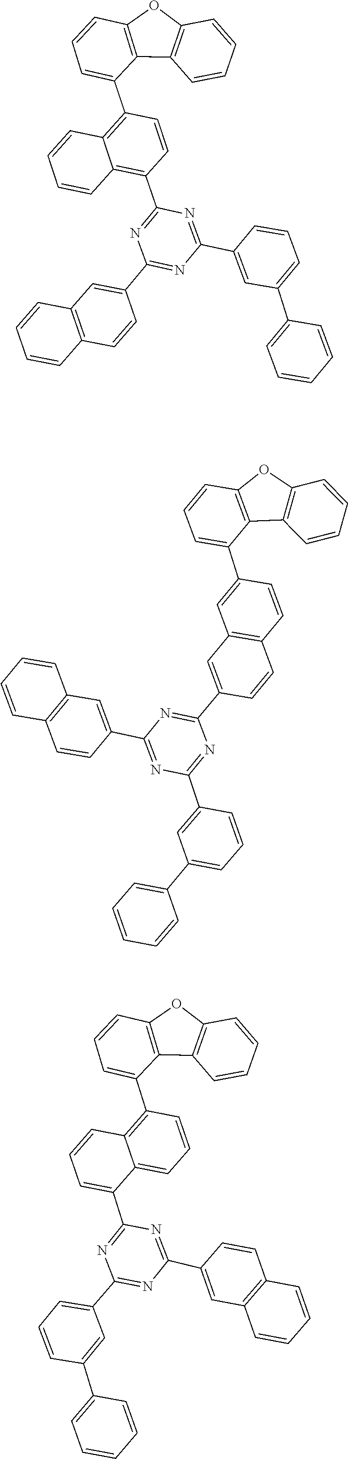 [SMILES: c1ccc(-c2cccc(-c3nc(-c4ccc5ccccc5c4)nc(-c4ccc(-c5cccc6oc7ccccc7c56)c5ccccc45)n3)c2)cc1 ; c1ccc(-c2cccc(-c3nc(-c4ccc5ccccc5c4)nc(-c4ccc5ccc(-c6cccc7oc8ccccc8c67)cc5c4)n3)c2)cc1 ; c1ccc(-c2cccc(-c3nc(-c4ccc5ccccc5c4)nc(-c4cccc5c(-c6cccc7oc8ccccc8c67)cccc45)n3)c2)cc1]

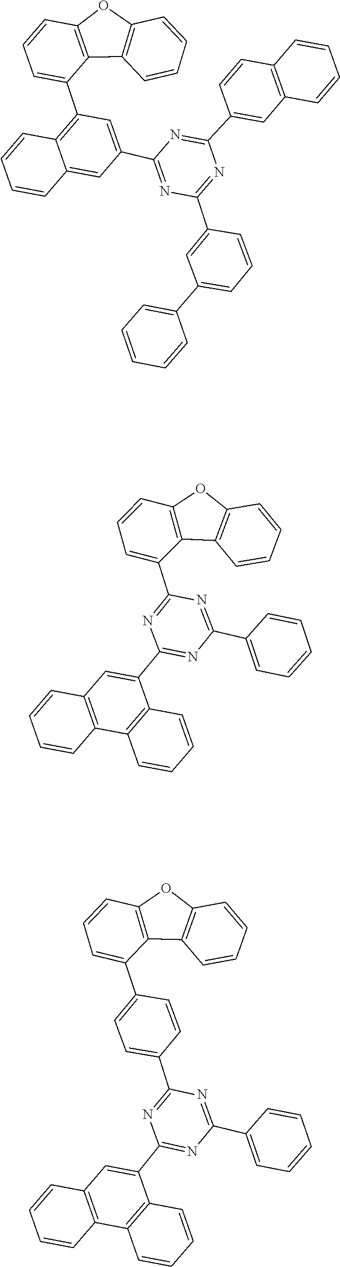 [SMILES: c1ccc(-c2cccc(-c3nc(-c4ccc5ccccc5c4)nc(-c4cc(-c5cccc6oc7ccccc7c56)c5ccccc5c4)n3)c2)cc1 ; c1ccc(-c2nc(-c3cc4ccccc4c4ccccc34)nc(-c3cccc4oc5ccccc5c34)n2)cc1 ; c1ccc(-c2nc(-c3ccc(-c4cccc5oc6ccccc6c45)cc3)nc(-c3cc4ccccc4c4ccccc34)n2)cc1]

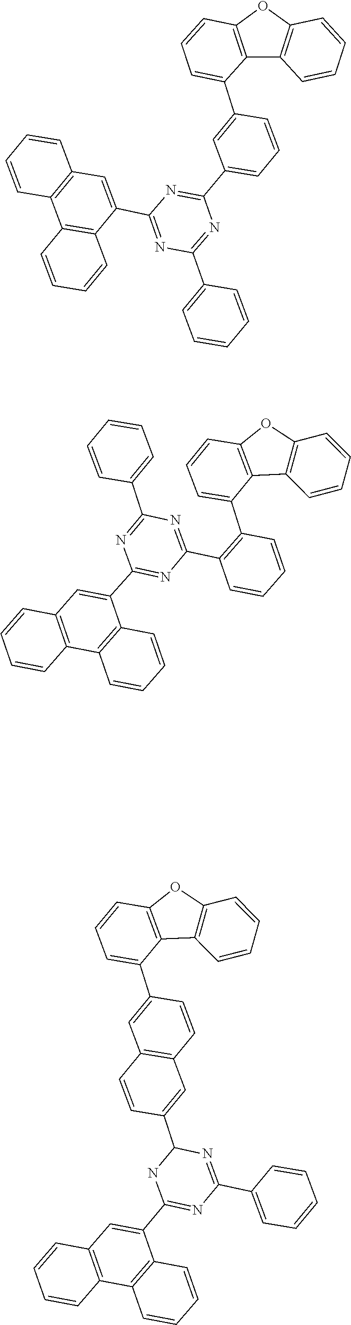

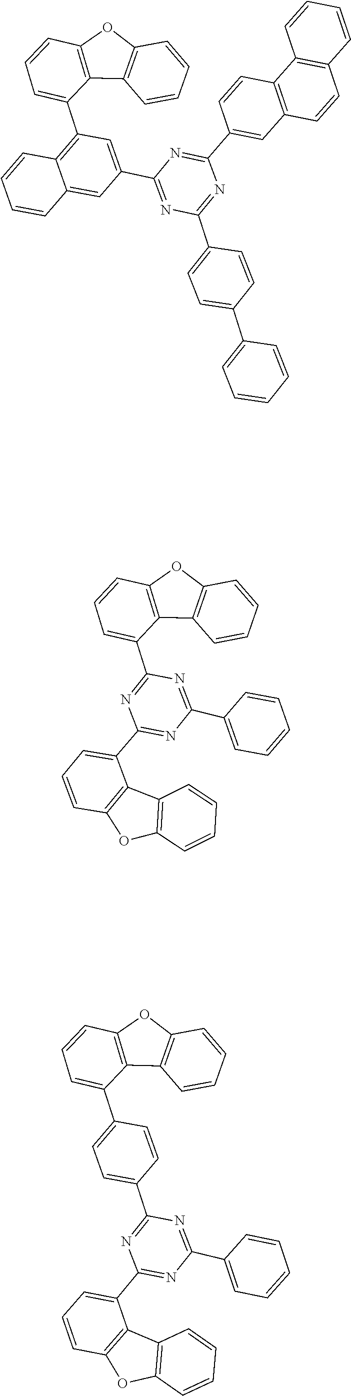 [SMILES: c1ccc(-c2ccc(-c3nc(-c4cc(-c5cccc6oc7ccccc7c56)c5ccccc5c4)nc(-c4ccc5c(ccc6ccccc65)c4)n3)cc2)cc1 ; c1ccc(-c2nc(-c3cccc4oc5ccccc5c34)nc(-c3cccc4oc5ccccc5c34)n2)cc1 ; c1ccc(-c2nc(-c3ccc(-c4cccc5oc6ccccc6c45)cc3)nc(-c3cccc4oc5ccccc5c34)n2)cc1]

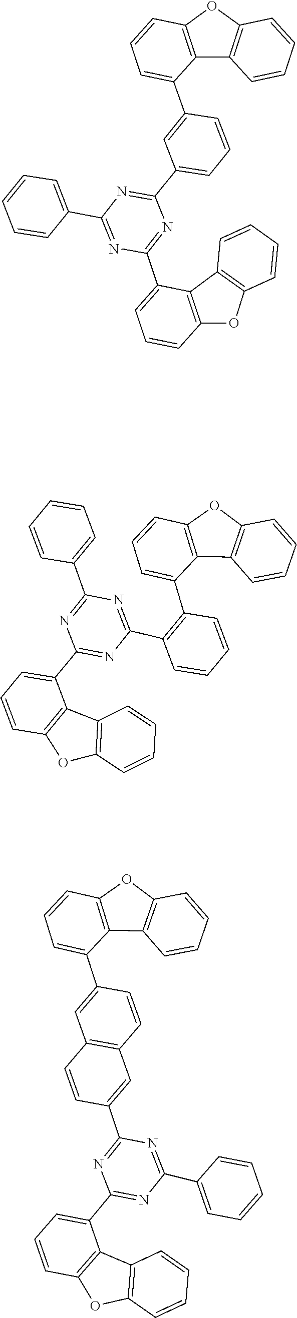 [SMILES: c1ccc(-c2nc(-c3cccc(-c4cccc5oc6ccccc6c45)c3)nc(-c3cccc4oc5ccccc5c34)n2)cc1 ; c1ccc(-c2nc(-c3ccccc3-c3cccc4oc5ccccc5c34)nc(-c3cccc4oc5ccccc5c34)n2)cc1 ; c1ccc(-c2nc(-c3ccc4cc(-c5cccc6oc7ccccc7c56)ccc4c3)nc(-c3cccc4oc5ccccc5c34)n2)cc1]

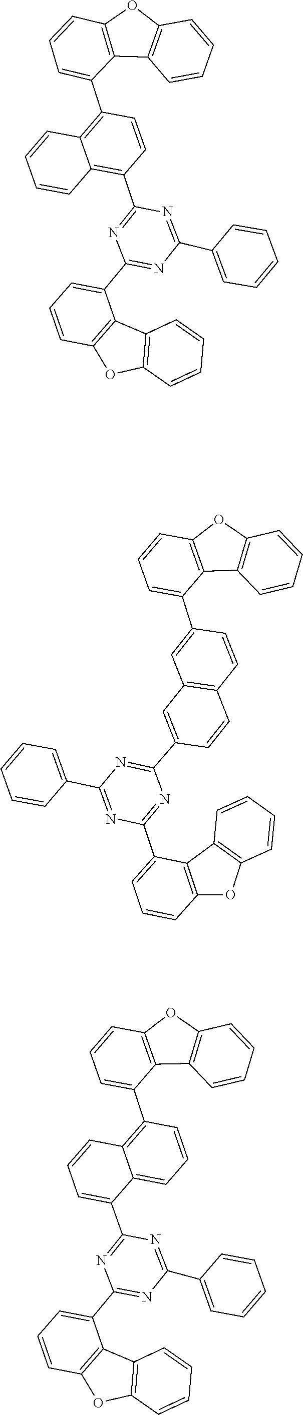 [SMILES: c1ccc(-c2nc(-c3ccc(-c4cccc5oc6ccccc6c45)c4ccccc34)nc(-c3cccc4oc5ccccc5c34)n2)cc1 ; c1ccc(-c2nc(-c3ccc4ccc(-c5cccc6oc7ccccc7c56)cc4c3)nc(-c3cccc4oc5ccccc5c34)n2)cc1 ; c1ccc(-c2nc(-c3cccc4c(-c5cccc6oc7ccccc7c56)cccc34)nc(-c3cccc4oc5ccccc5c34)n2)cc1]

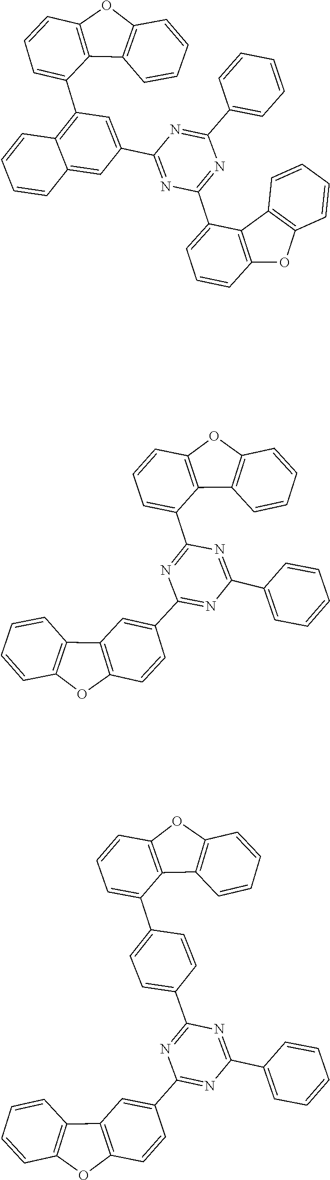 [SMILES: c1ccc(-c2nc(-c3cc(-c4cccc5oc6ccccc6c45)c4ccccc4c3)nc(-c3cccc4oc5ccccc5c34)n2)cc1 ; c1ccc(-c2nc(-c3ccc4oc5ccccc5c4c3)nc(-c3cccc4oc5ccccc5c34)n2)cc1 ; c1ccc(-c2nc(-c3ccc(-c4cccc5oc6ccccc6c45)cc3)nc(-c3ccc4oc5ccccc5c4c3)n2)cc1]

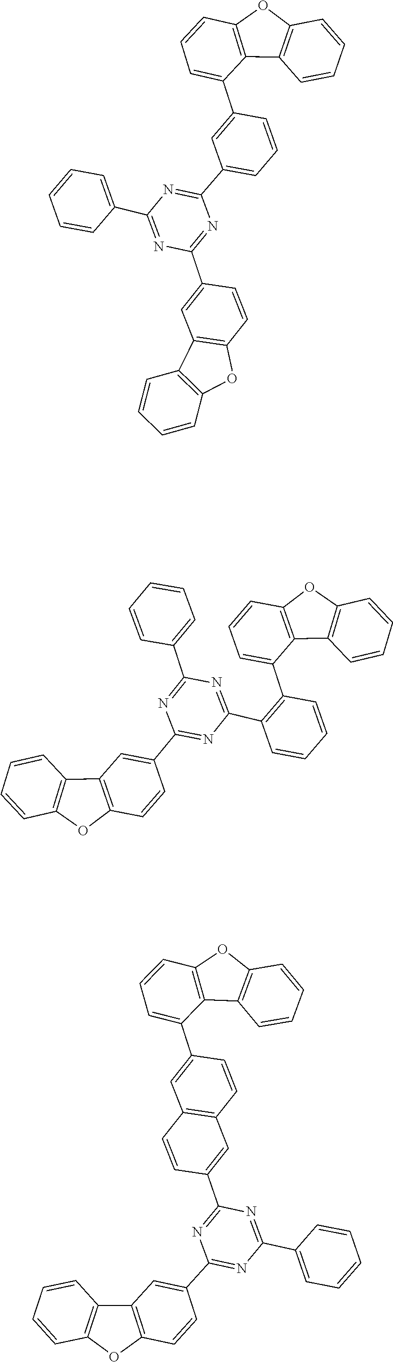 [SMILES: c1ccc(-c2nc(-c3cccc(-c4cccc5oc6ccccc6c45)c3)nc(-c3ccc4oc5ccccc5c4c3)n2)cc1 ; c1ccc(-c2nc(-c3ccc4oc5ccccc5c4c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4oc5ccccc5c4c3)n2)cc1]

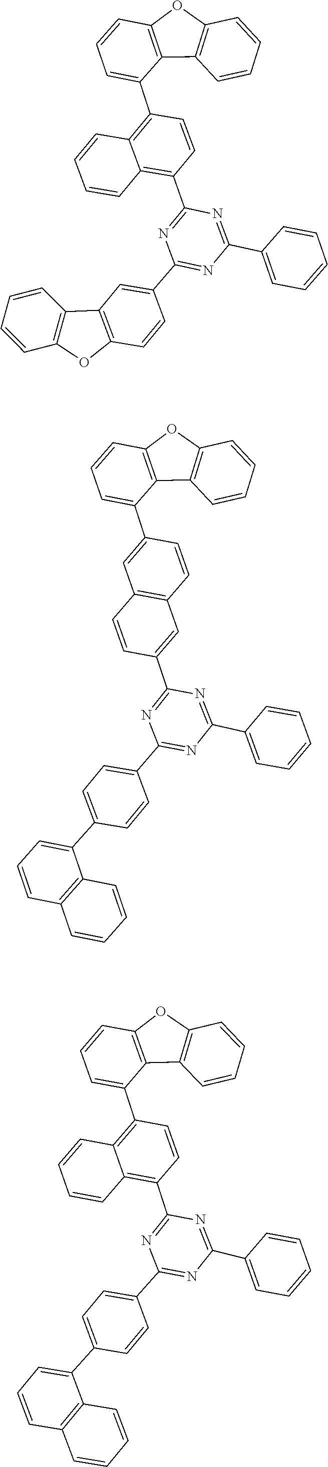 [SMILES: c1ccc(-c2nc(-c3ccc4oc5ccccc5c4c3)nc(-c3ccc(-c4cccc5oc6ccccc6c45)c4ccccc34)n2)cc1 ; c1ccc(-c2nc(-c3ccc(-c4cccc5ccccc45)cc3)nc(-c3ccc4cc(-c5cccc6oc7ccccc7c56)ccc4c3)n2)cc1 ; c1ccc(-c2nc(-c3ccc(-c4cccc5ccccc45)cc3)nc(-c3ccc(-c4cccc5oc6ccccc6c45)c4ccccc34)n2)cc1]

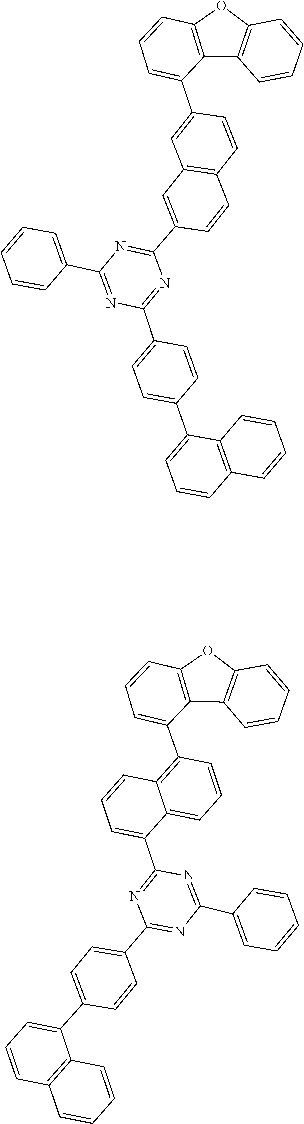 [SMILES: c1ccc(-c2nc(-c3ccc(-c4cccc5ccccc45)cc3)nc(-c3ccc4ccc(-c5cccc6oc7ccccc7c56)cc4c3)n2)cc1 ; c1ccc(-c2nc(-c3ccc(-c4cccc5ccccc45)cc3)nc(-c3cccc4c(-c5cccc6oc7ccccc7c56)cccc34)n2)cc1]

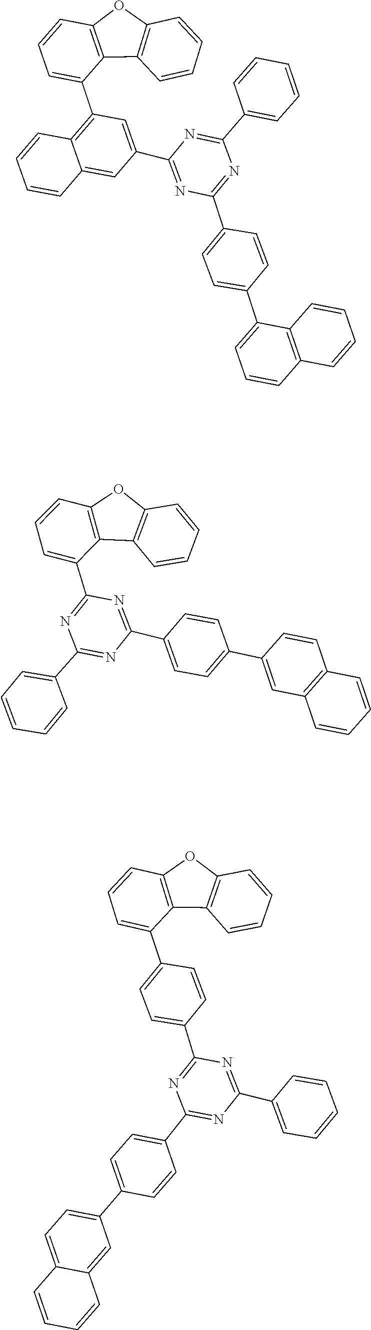 [SMILES: c1ccc(-c2nc(-c3ccc(-c4cccc5ccccc45)cc3)nc(-c3cc(-c4cccc5oc6ccccc6c45)c4ccccc4c3)n2)cc1 ; c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc(-c4cccc5oc6ccccc6c45)cc3)n2)cc1]

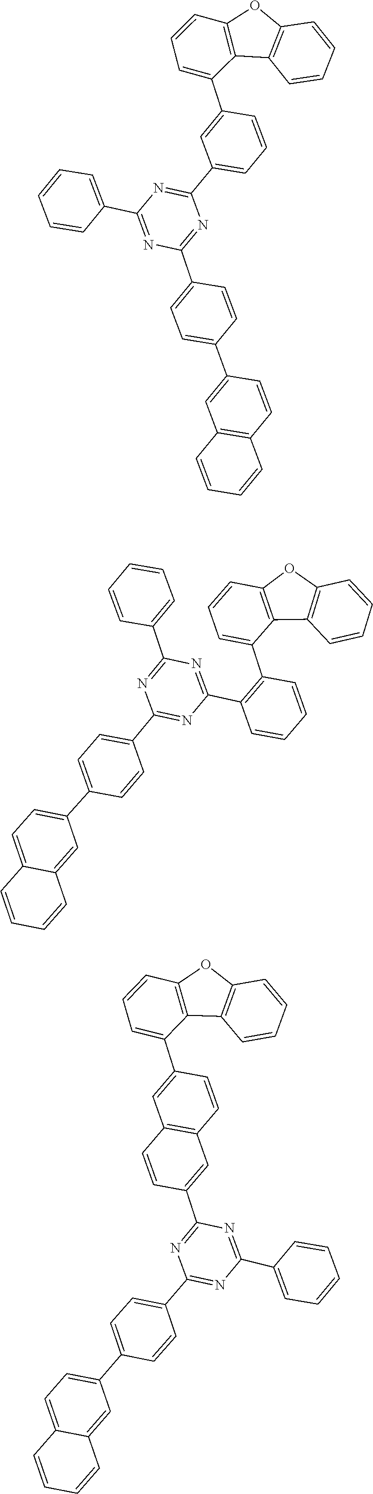 [SMILES: c1ccc(-c2nc(-c3ccc(-c4ccc5ccccc5c4)cc3)nc(-c3cccc(-c4cccc5oc6ccccc6c45)c3)n2)cc1 ; c1ccc(-c2nc(-c3ccc(-c4ccc5ccccc5c4)cc3)nc(-c3ccccc3-c3cccc4oc5ccccc5c34)n2)cc1 ; c1ccc(-c2nc(-c3ccc(-c4ccc5ccccc5c4)cc3)nc(-c3ccc4cc(-c5cccc6oc7ccccc7c56)ccc4c3)n2)cc1]

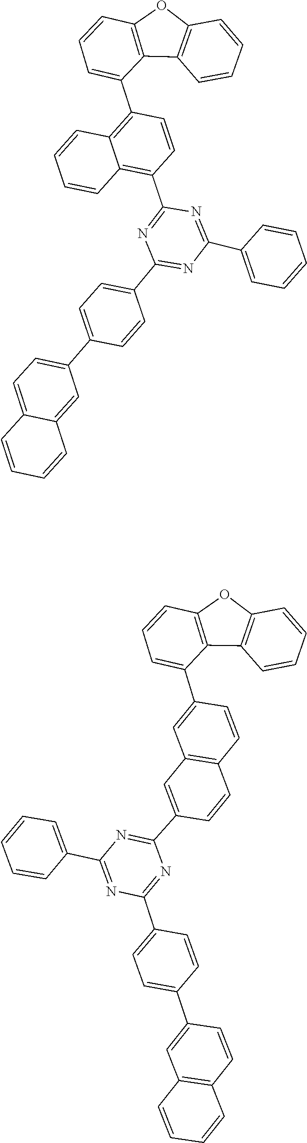 [SMILES: c1ccc(-c2nc(-c3ccc(-c4ccc5ccccc5c4)cc3)nc(-c3ccc(-c4cccc5oc6ccccc6c45)c4ccccc34)n2)cc1 ; c1ccc(-c2nc(-c3ccc(-c4ccc5ccccc5c4)cc3)nc(-c3ccc4ccc(-c5cccc6oc7ccccc7c56)cc4c3)n2)cc1]

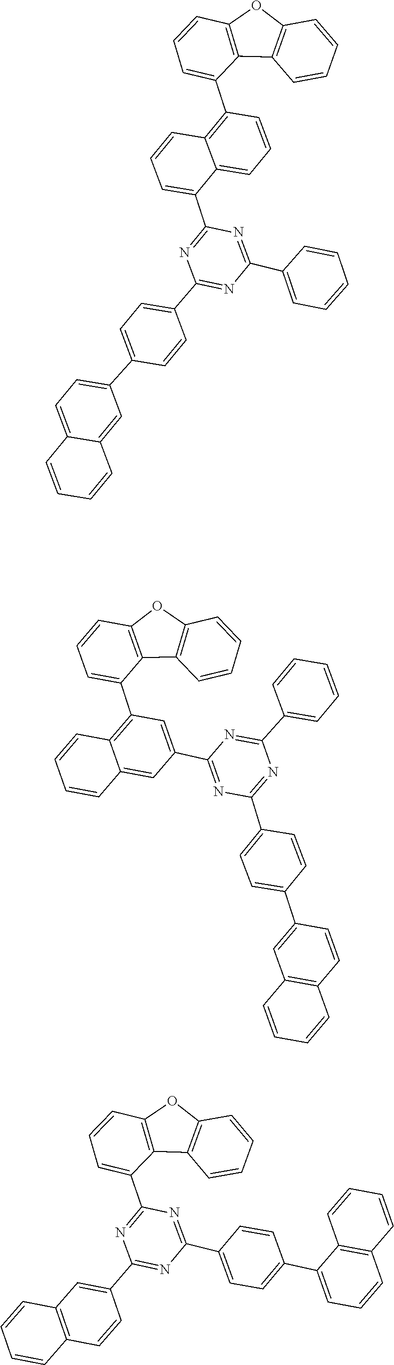 [SMILES: c1ccc(-c2nc(-c3ccc(-c4ccc5ccccc5c4)cc3)nc(-c3cccc4c(-c5cccc6oc7ccccc7c56)cccc34)n2)cc1 ; c1ccc(-c2nc(-c3ccc(-c4ccc5ccccc5c4)cc3)nc(-c3cc(-c4cccc5oc6ccccc6c45)c4ccccc4c3)n2)cc1 ; c1ccc2cc(-c3nc(-c4ccc(-c5cccc6ccccc56)cc4)nc(-c4cccc5oc6ccccc6c45)n3)ccc2c1]

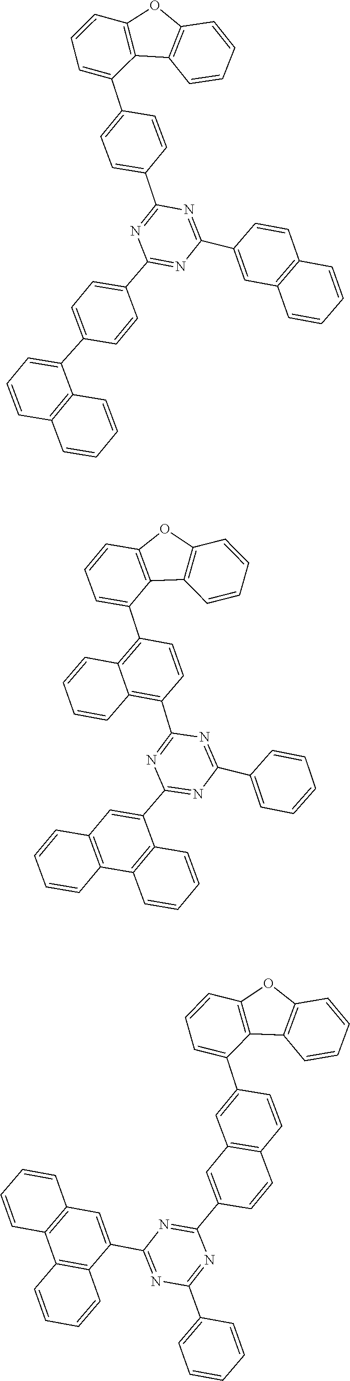 [SMILES: c1ccc2cc(-c3nc(-c4ccc(-c5cccc6ccccc56)cc4)nc(-c4ccc(-c5cccc6oc7ccccc7c56)cc4)n3)ccc2c1 ; c1ccc(-c2nc(-c3ccc(-c4cccc5oc6ccccc6c45)c4ccccc34)nc(-c3cc4ccccc4c4ccccc34)n2)cc1 ; c1ccc(-c2nc(-c3ccc4ccc(-c5cccc6oc7ccccc7c56)cc4c3)nc(-c3cc4ccccc4c4ccccc34)n2)cc1]

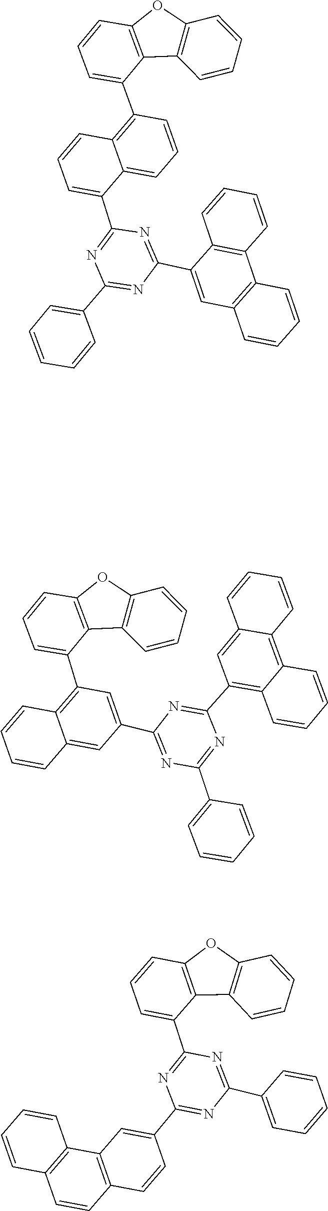 [SMILES: c1ccc(-c2nc(-c3cccc4c(-c5cccc6oc7ccccc7c56)cccc34)nc(-c3cc4ccccc4c4ccccc34)n2)cc1 ; c1ccc(-c2nc(-c3cc(-c4cccc5oc6ccccc6c45)c4ccccc4c3)nc(-c3cc4ccccc4c4ccccc34)n2)cc1 ; c1ccc(-c2nc(-c3ccc4ccc5ccccc5c4c3)nc(-c3cccc4oc5ccccc5c34)n2)cc1]

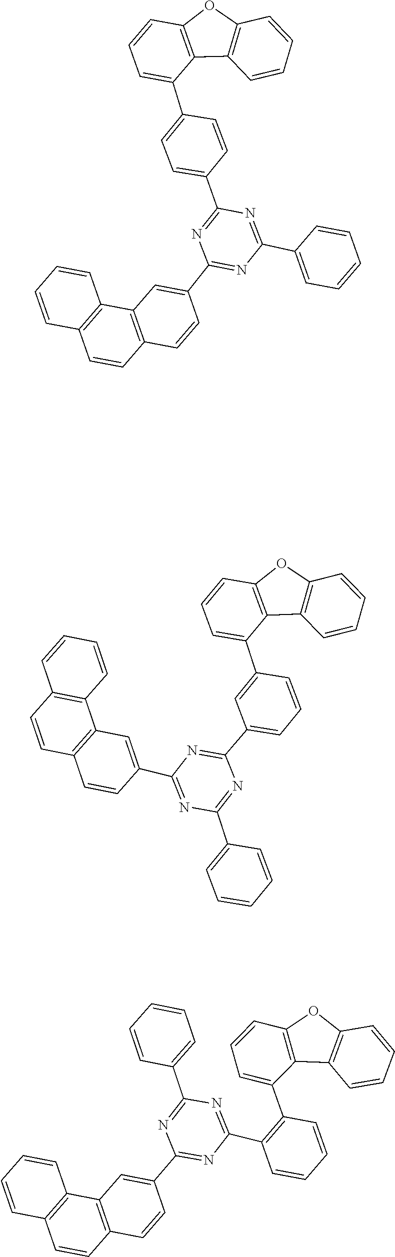 [SMILES: c1ccc(-c2nc(-c3ccc(-c4cccc5oc6ccccc6c45)cc3)nc(-c3ccc4ccc5ccccc5c4c3)n2)cc1 ; c1ccc(-c2nc(-c3cccc(-c4cccc5oc6ccccc6c45)c3)nc(-c3ccc4ccc5ccccc5c4c3)n2)cc1 ; c1ccc(-c2nc(-c3ccc4ccc5ccccc5c4c3)nc(-c3ccccc3-c3cccc4oc5ccccc5c34)n2)cc1]

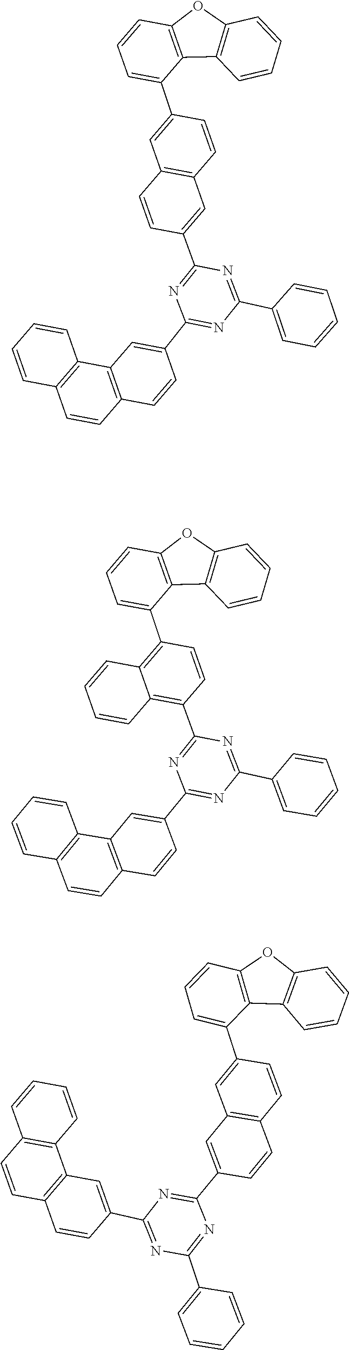 [SMILES: c1ccc(-c2nc(-c3ccc4cc(-c5cccc6oc7ccccc7c56)ccc4c3)nc(-c3ccc4ccc5ccccc5c4c3)n2)cc1 ; c1ccc(-c2nc(-c3ccc4ccc5ccccc5c4c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4ccc5ccccc5c4c3)n2)cc1]

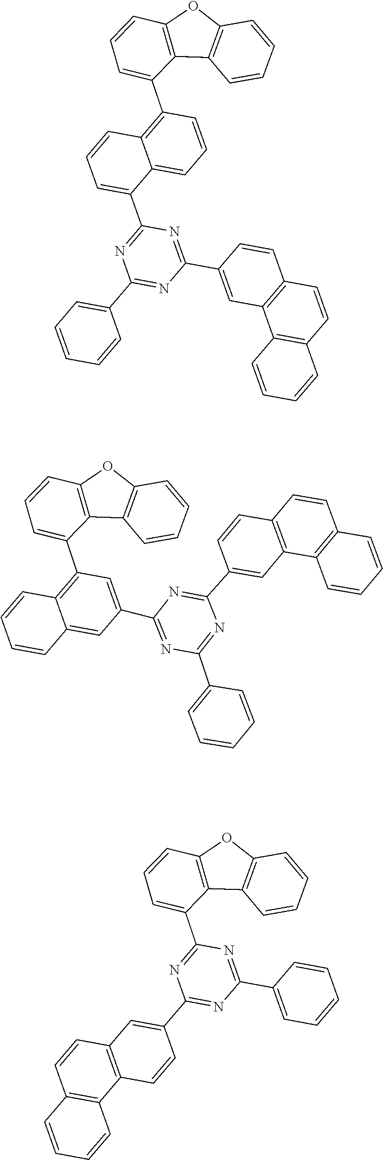 [SMILES: c1ccc(-c2nc(-c3ccc4ccc5ccccc5c4c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4ccc5ccccc5c4c3)n2)cc1 ; c1ccc(-c2nc(-c3ccc4c(ccc5ccccc54)c3)nc(-c3cccc4oc5ccccc5c34)n2)cc1]

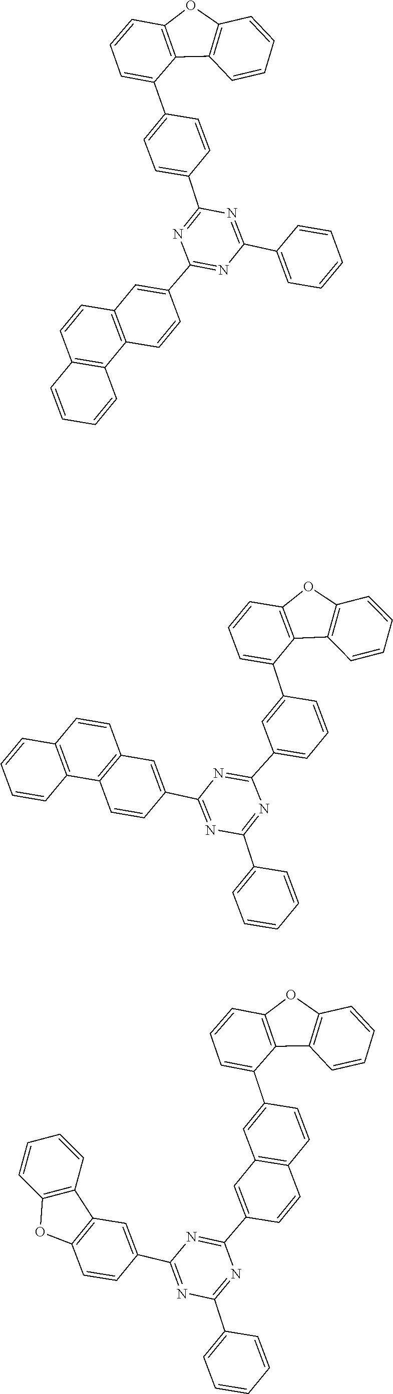 [SMILES: c1ccc(-c2nc(-c3ccc(-c4cccc5oc6ccccc6c45)cc3)nc(-c3ccc4c(ccc5ccccc54)c3)n2)cc1 ; c1ccc(-c2nc(-c3cccc(-c4cccc5oc6ccccc6c45)c3)nc(-c3ccc4c(ccc5ccccc54)c3)n2)cc1 ; c1ccc(-c2nc(-c3ccc4ccc(-c5cccc6oc7ccccc7c56)cc4c3)nc(-c3ccc4oc5ccccc5c4c3)n2)cc1]

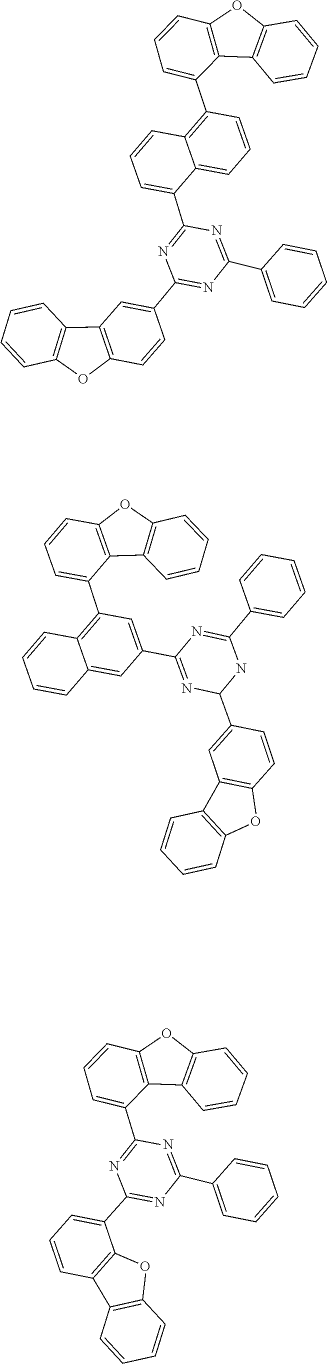

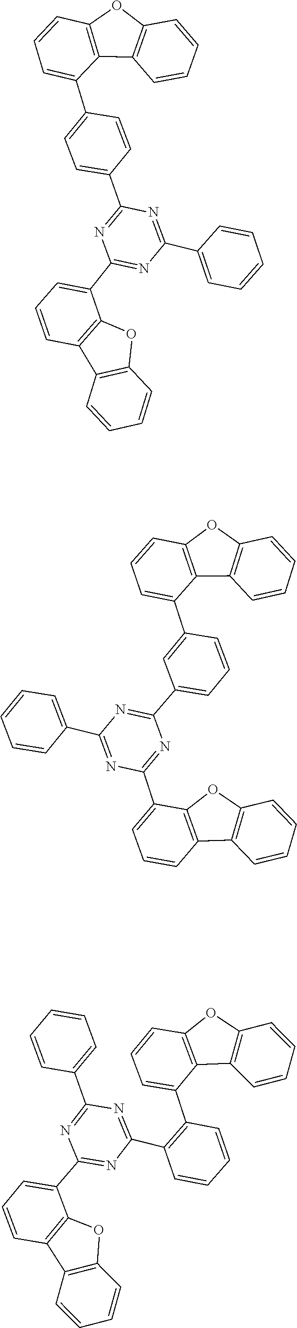 [SMILES: c1ccc(-c2nc(-c3ccc(-c4cccc5oc6ccccc6c45)cc3)nc(-c3cccc4c3oc3ccccc34)n2)cc1 ; c1ccc(-c2nc(-c3cccc(-c4cccc5oc6ccccc6c45)c3)nc(-c3cccc4c3oc3ccccc34)n2)cc1 ; c1ccc(-c2nc(-c3ccccc3-c3cccc4oc5ccccc5c34)nc(-c3cccc4c3oc3ccccc34)n2)cc1]

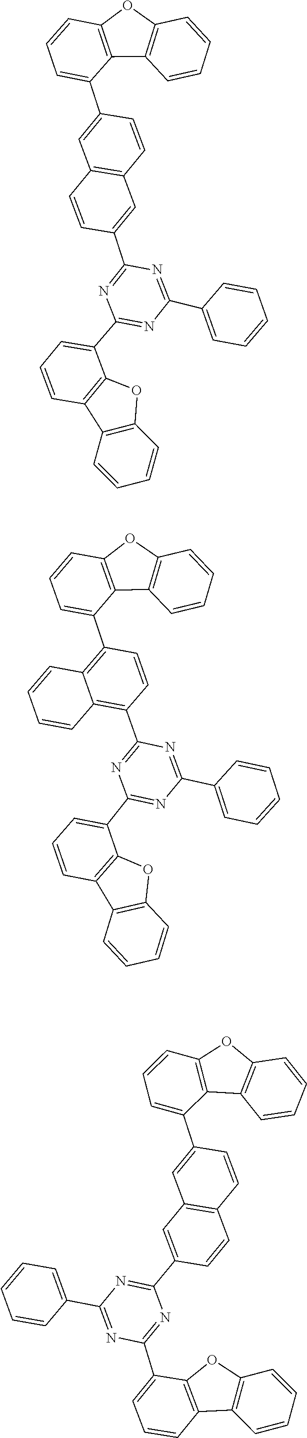 [SMILES: c1ccc(-c2nc(-c3ccc4cc(-c5cccc6oc7ccccc7c56)ccc4c3)nc(-c3cccc4c3oc3ccccc34)n2)cc1 ; c1ccc(-c2nc(-c3ccc(-c4cccc5oc6ccccc6c45)c4ccccc34)nc(-c3cccc4c3oc3ccccc34)n2)cc1 ; c1ccc(-c2nc(-c3ccc4ccc(-c5cccc6oc7ccccc7c56)cc4c3)nc(-c3cccc4c3oc3ccccc34)n2)cc1]

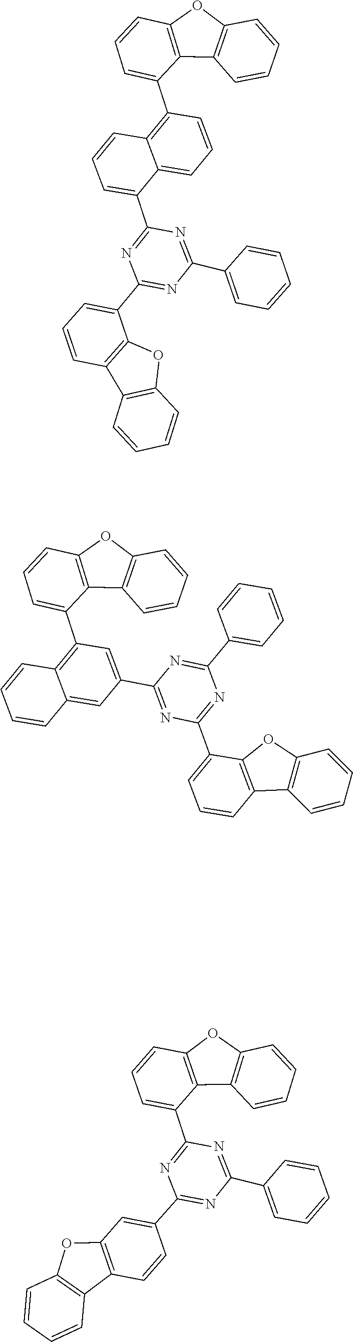 [SMILES: c1ccc(-c2nc(-c3cccc4c(-c5cccc6oc7ccccc7c56)cccc34)nc(-c3cccc4c3oc3ccccc34)n2)cc1 ; c1ccc(-c2nc(-c3cc(-c4cccc5oc6ccccc6c45)c4ccccc4c3)nc(-c3cccc4c3oc3ccccc34)n2)cc1 ; c1ccc(-c2nc(-c3ccc4c(c3)oc3ccccc34)nc(-c3cccc4oc5ccccc5c34)n2)cc1]

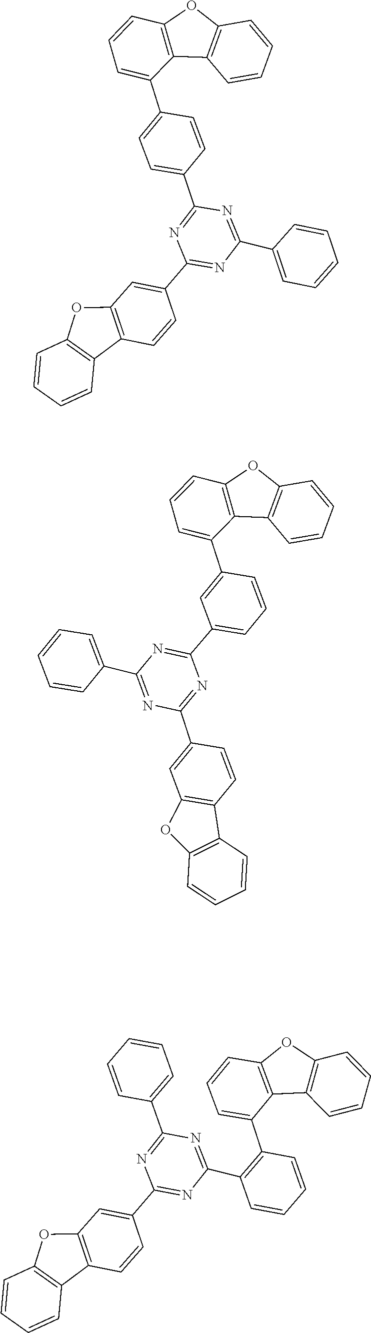 [SMILES: c1ccc(-c2nc(-c3ccc(-c4cccc5oc6ccccc6c45)cc3)nc(-c3ccc4c(c3)oc3ccccc34)n2)cc1 ; c1ccc(-c2nc(-c3cccc(-c4cccc5oc6ccccc6c45)c3)nc(-c3ccc4c(c3)oc3ccccc34)n2)cc1 ; c1ccc(-c2nc(-c3ccc4c(c3)oc3ccccc34)nc(-c3ccccc3-c3cccc4oc5ccccc5c34)n2)cc1]

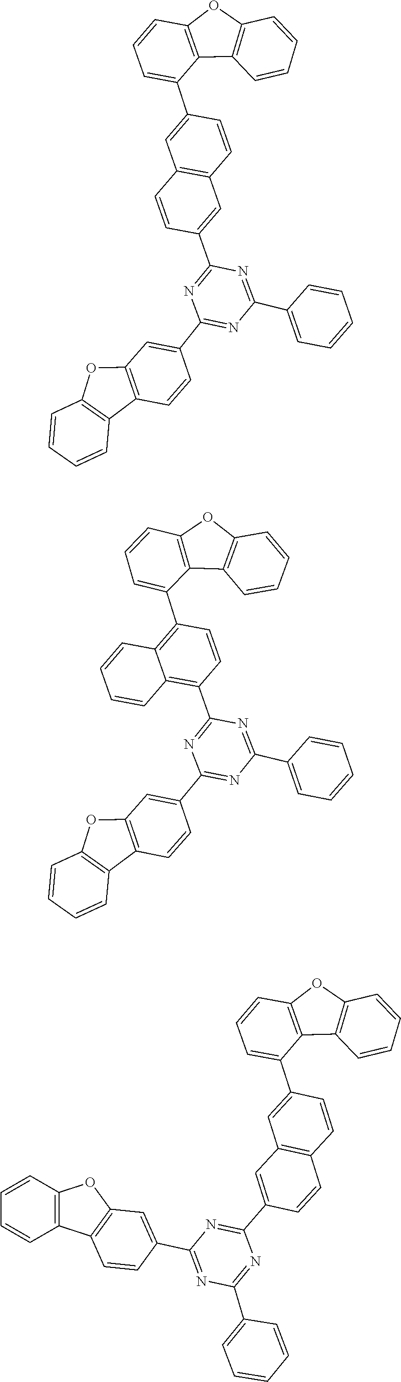 [SMILES: c1ccc(-c2nc(-c3ccc4cc(-c5cccc6oc7ccccc7c56)ccc4c3)nc(-c3ccc4c(c3)oc3ccccc34)n2)cc1 ; c1ccc(-c2nc(-c3ccc4c(c3)oc3ccccc34)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(c3)oc3ccccc34)n2)cc1]

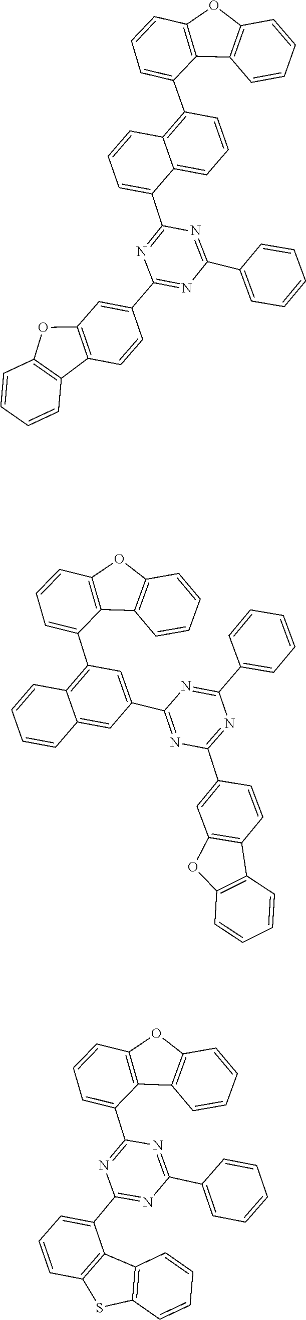 [SMILES: c1ccc(-c2nc(-c3ccc4c(c3)oc3ccccc34)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(c3)oc3ccccc34)n2)cc1 ; c1ccc(-c2nc(-c3cccc4oc5ccccc5c34)nc(-c3cccc4sc5ccccc5c34)n2)cc1]

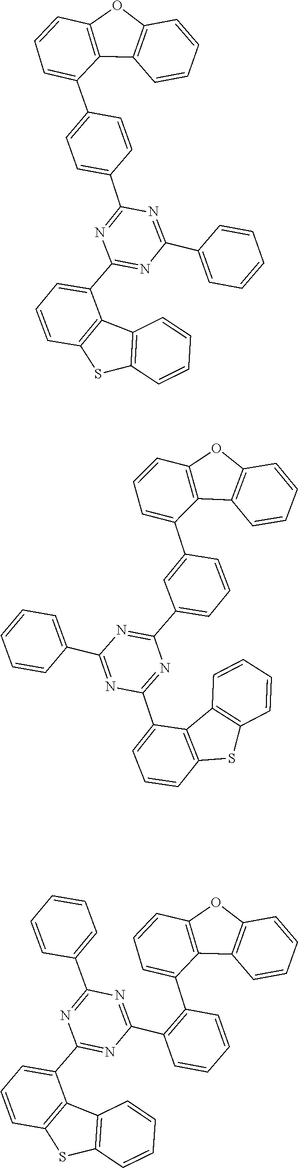 [SMILES: c1ccc(-c2nc(-c3ccc(-c4cccc5oc6ccccc6c45)cc3)nc(-c3cccc4sc5ccccc5c34)n2)cc1 ; c1ccc(-c2nc(-c3cccc(-c4cccc5oc6ccccc6c45)c3)nc(-c3cccc4sc5ccccc5c34)n2)cc1 ; c1ccc(-c2nc(-c3ccccc3-c3cccc4oc5ccccc5c34)nc(-c3cccc4sc5ccccc5c34)n2)cc1]

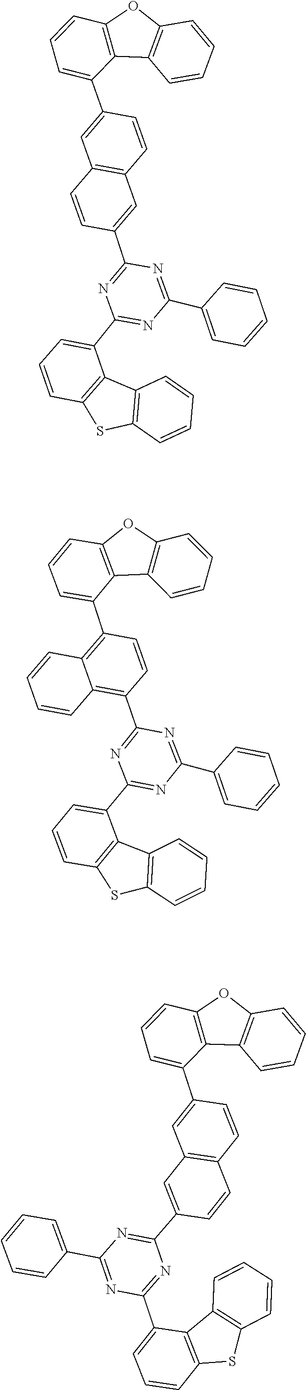 [SMILES: c1ccc(-c2nc(-c3ccc4cc(-c5cccc6oc7ccccc7c56)ccc4c3)nc(-c3cccc4sc5ccccc5c34)n2)cc1 ; c1ccc(-c2nc(-c3ccc(-c4cccc5oc6ccccc6c45)c4ccccc34)nc(-c3cccc4sc5ccccc5c34)n2)cc1 ; c1ccc(-c2nc(-c3ccc4ccc(-c5cccc6oc7ccccc7c56)cc4c3)nc(-c3cccc4sc5ccccc5c34)n2)cc1]

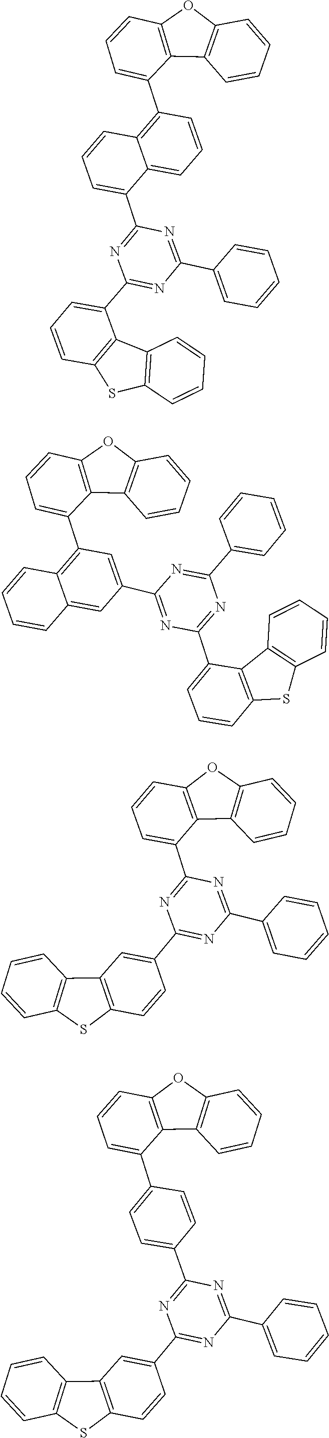

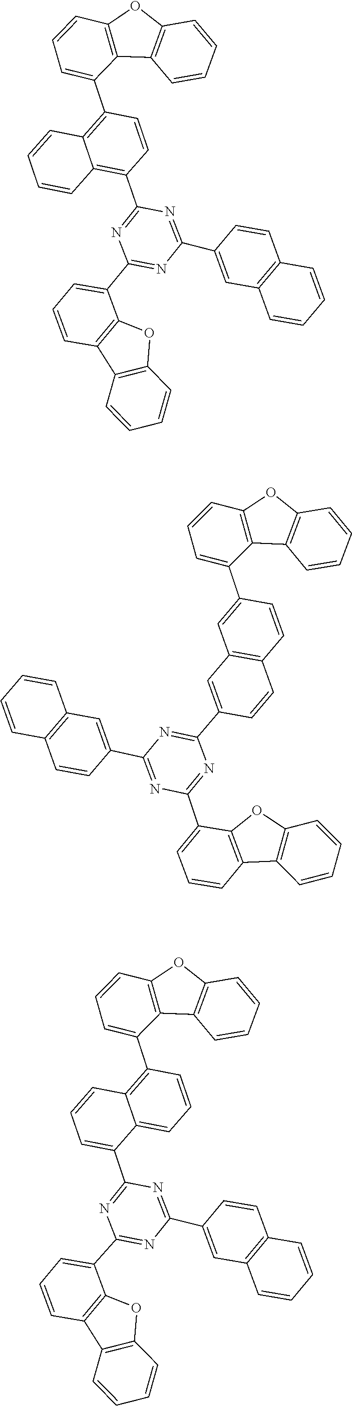 [SMILES: c1ccc2cc(-c3nc(-c4ccc(-c5cccc6oc7ccccc7c56)c5ccccc45)nc(-c4cccc5c4oc4ccccc45)n3)ccc2c1 ; c1ccc2cc(-c3nc(-c4ccc5ccc(-c6cccc7oc8ccccc8c67)cc5c4)nc(-c4cccc5c4oc4ccccc45)n3)ccc2c1 ; c1ccc2cc(-c3nc(-c4cccc5c(-c6cccc7oc8ccccc8c67)cccc45)nc(-c4cccc5c4oc4ccccc45)n3)ccc2c1]

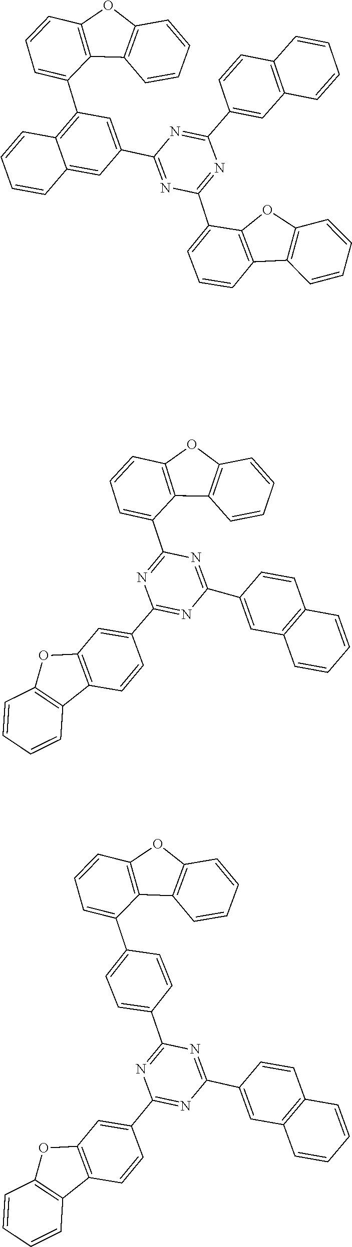 [SMILES: c1ccc2cc(-c3nc(-c4cc(-c5cccc6oc7ccccc7c56)c5ccccc5c4)nc(-c4cccc5c4oc4ccccc45)n3)ccc2c1 ; c1ccc2cc(-c3nc(-c4ccc5c(c4)oc4ccccc45)nc(-c4cccc5oc6ccccc6c45)n3)ccc2c1 ; c1ccc2cc(-c3nc(-c4ccc(-c5cccc6oc7ccccc7c56)cc4)nc(-c4ccc5c(c4)oc4ccccc45)n3)ccc2c1]

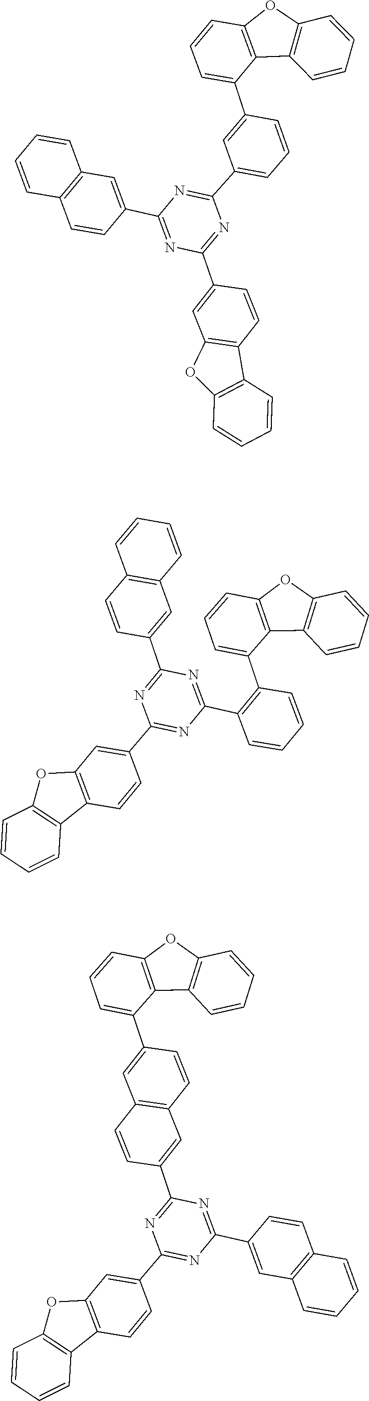 [SMILES: c1cc(-c2nc(-c3ccc4ccccc4c3)nc(-c3ccc4c(c3)oc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3ccc4c(c3)oc3ccccc34)n2)c1 ; c1ccc2cc(-c3nc(-c4ccc5cc(-c6cccc7oc8ccccc8c67)ccc5c4)nc(-c4ccc5c(c4)oc4ccccc45)n3)ccc2c1]

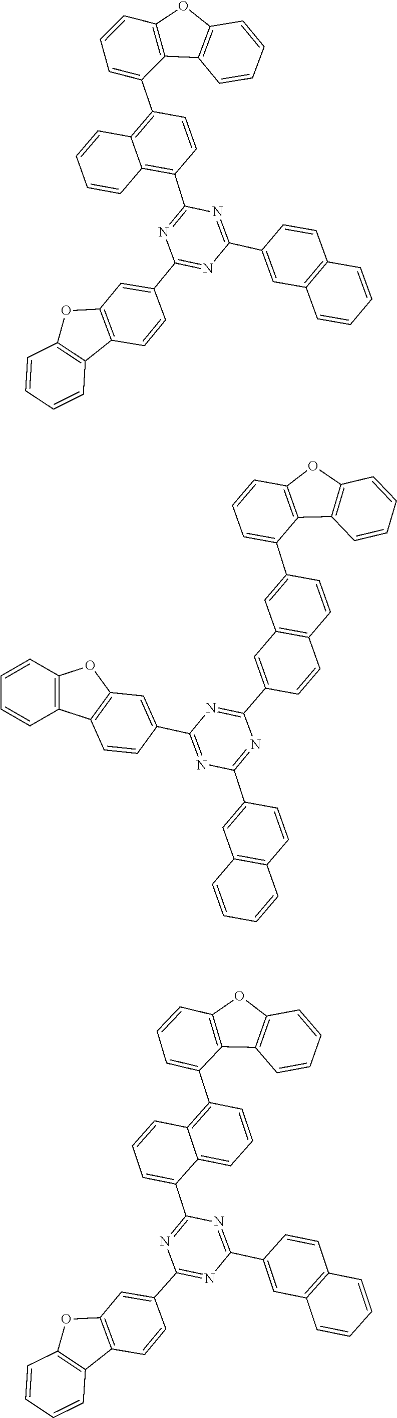 [SMILES: c1ccc2cc(-c3nc(-c4ccc5c(c4)oc4ccccc45)nc(-c4ccc(-c5cccc6oc7ccccc7c56)c5ccccc45)n3)ccc2c1 ; c1ccc2cc(-c3nc(-c4ccc5ccc(-c6cccc7oc8ccccc8c67)cc5c4)nc(-c4ccc5c(c4)oc4ccccc45)n3)ccc2c1 ; c1ccc2cc(-c3nc(-c4ccc5c(c4)oc4ccccc45)nc(-c4cccc5c(-c6cccc7oc8ccccc8c67)cccc45)n3)ccc2c1]

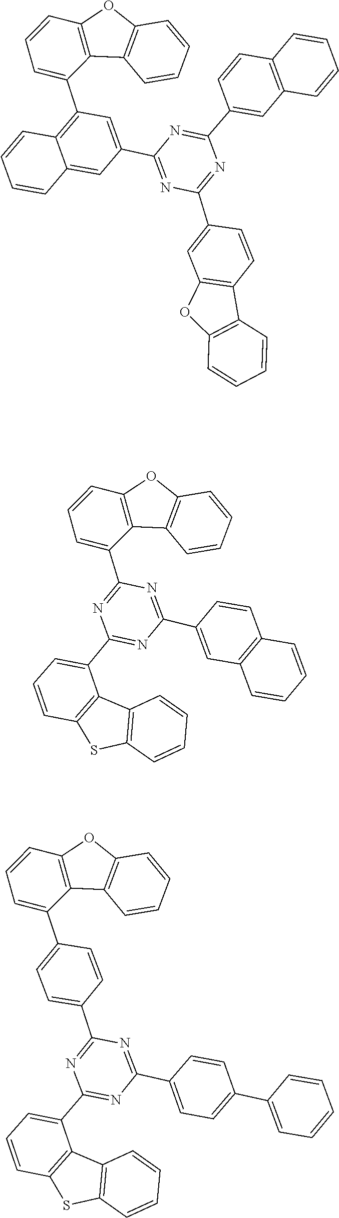 [SMILES: c1ccc2cc(-c3nc(-c4cc(-c5cccc6oc7ccccc7c56)c5ccccc5c4)nc(-c4ccc5c(c4)oc4ccccc45)n3)ccc2c1 ; c1ccc2cc(-c3nc(-c4cccc5oc6ccccc6c45)nc(-c4cccc5sc6ccccc6c45)n3)ccc2c1 ; c1ccc(-c2ccc(-c3nc(-c4ccc(-c5cccc6oc7ccccc7c56)cc4)nc(-c4cccc5sc6ccccc6c45)n3)cc2)cc1]

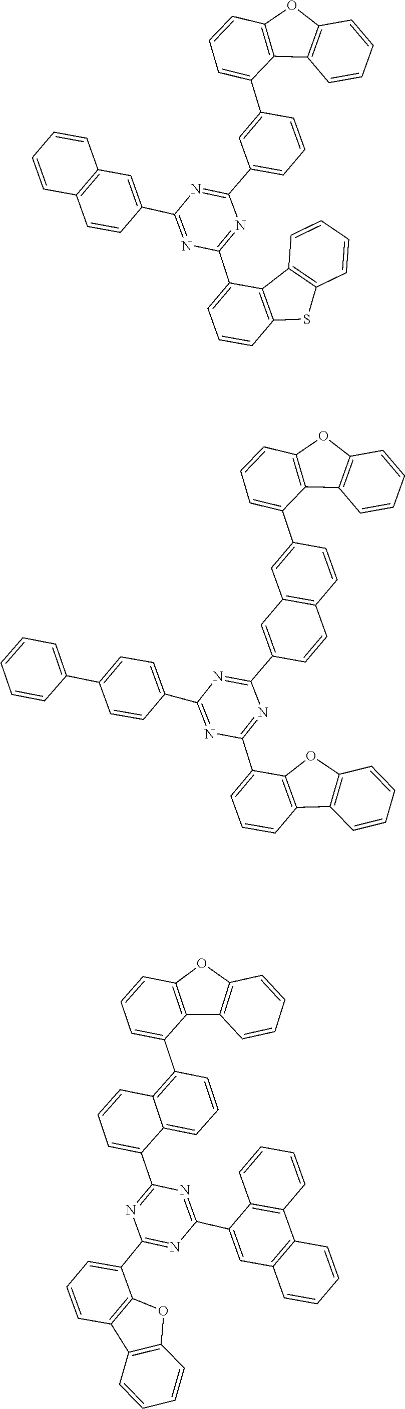 [SMILES: c1cc(-c2nc(-c3ccc4ccccc4c3)nc(-c3cccc4sc5ccccc5c34)n2)cc(-c2cccc3oc4ccccc4c23)c1 ; c1ccc(-c2ccc(-c3nc(-c4ccc5ccc(-c6cccc7oc8ccccc8c67)cc5c4)nc(-c4cccc5c4oc4ccccc45)n3)cc2)cc1 ; c1ccc2c(c1)cc(-c1nc(-c3cccc4c(-c5cccc6oc7ccccc7c56)cccc34)nc(-c3cccc4c3oc3ccccc34)n1)c1ccccc12]

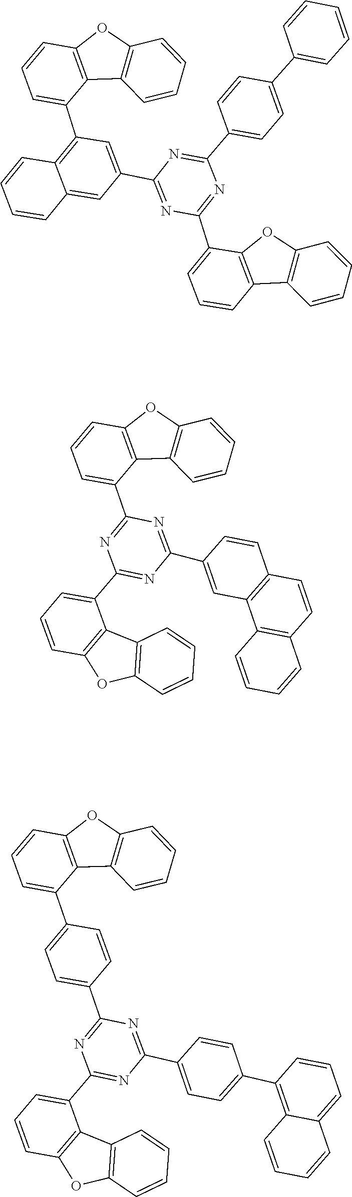 [SMILES: c1ccc(-c2ccc(-c3nc(-c4cc(-c5cccc6oc7ccccc7c56)c5ccccc5c4)nc(-c4cccc5c4oc4ccccc45)n3)cc2)cc1 ; c1ccc2c(c1)ccc1ccc(-c3nc(-c4cccc5oc6ccccc6c45)nc(-c4cccc5oc6ccccc6c45)n3)cc12 ; c1ccc2c(-c3ccc(-c4nc(-c5ccc(-c6cccc7oc8ccccc8c67)cc5)nc(-c5cccc6oc7ccccc7c56)n4)cc3)cccc2c1]

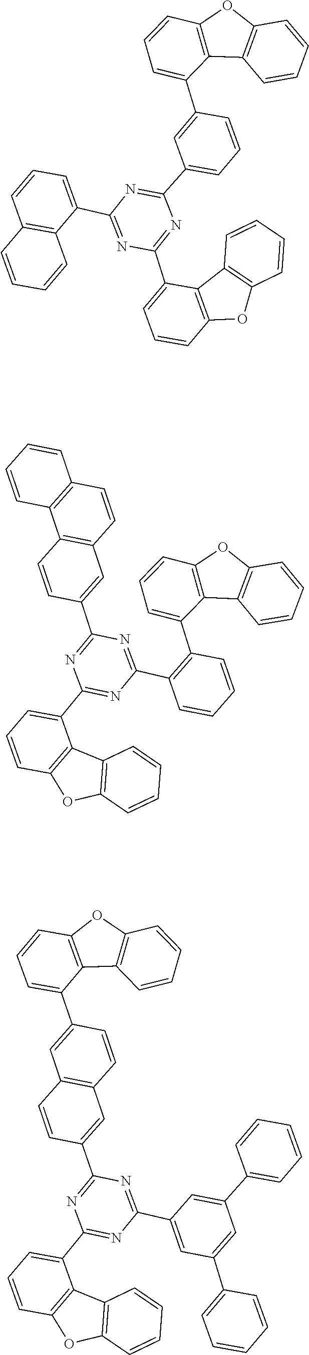 [SMILES: c1cc(-c2nc(-c3cccc4ccccc34)nc(-c3cccc4oc5ccccc5c34)n2)cc(-c2cccc3oc4ccccc4c23)c1 ; c1ccc(-c2cccc3oc4ccccc4c23)c(-c2nc(-c3ccc4c(ccc5ccccc54)c3)nc(-c3cccc4oc5ccccc5c34)n2)c1 ; c1ccc(-c2cc(-c3ccccc3)cc(-c3nc(-c4ccc5cc(-c6cccc7oc8ccccc8c67)ccc5c4)nc(-c4cccc5oc6ccccc6c45)n3)c2)cc1]

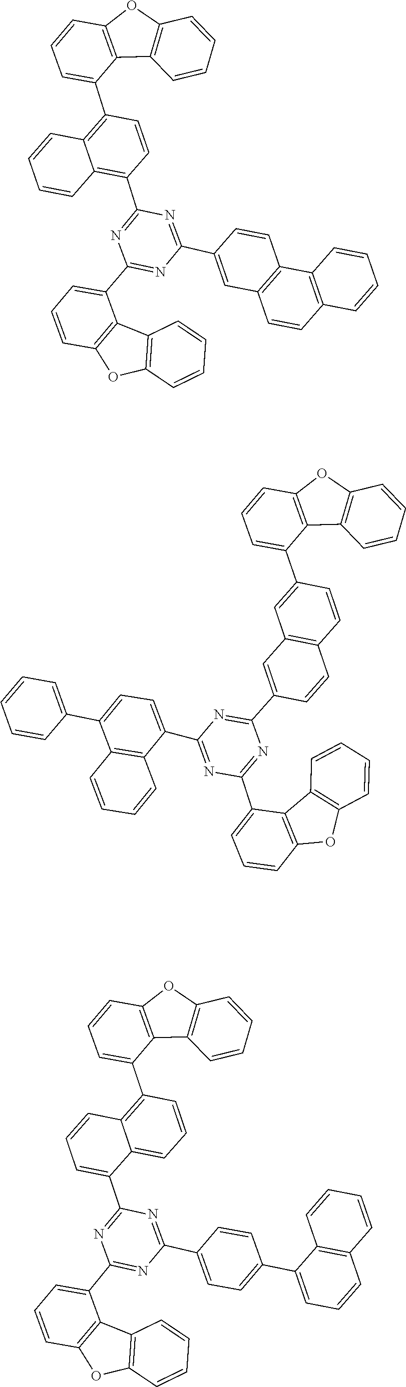 [SMILES: c1ccc2c(c1)ccc1cc(-c3nc(-c4ccc(-c5cccc6oc7ccccc7c56)c5ccccc45)nc(-c4cccc5oc6ccccc6c45)n3)ccc12 ; c1ccc(-c2ccc(-c3nc(-c4ccc5ccc(-c6cccc7oc8ccccc8c67)cc5c4)nc(-c4cccc5oc6ccccc6c45)n3)c3ccccc23)cc1 ; c1ccc2c(-c3ccc(-c4nc(-c5cccc6c(-c7cccc8oc9ccccc9c78)cccc56)nc(-c5cccc6oc7ccccc7c56)n4)cc3)cccc2c1]

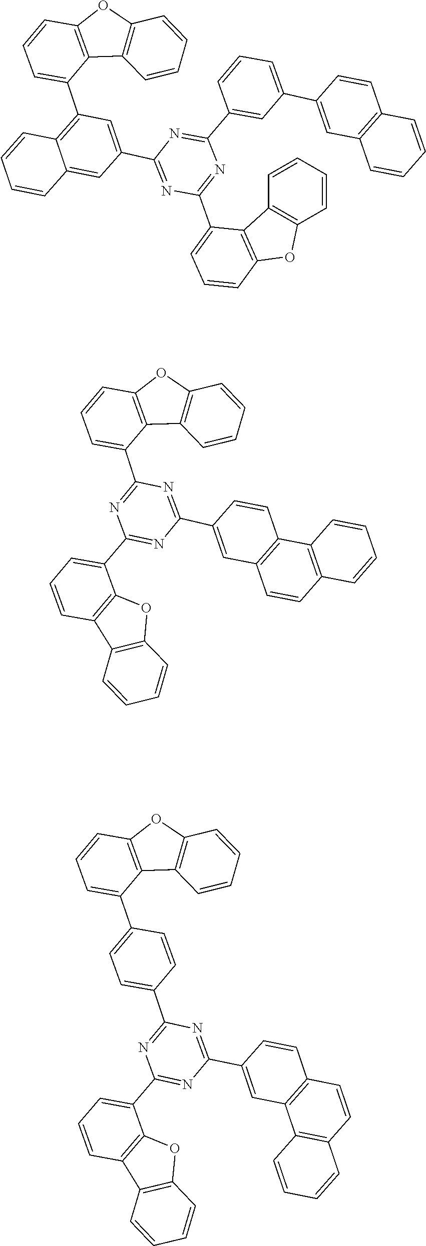 [SMILES: c1cc(-c2ccc3ccccc3c2)cc(-c2nc(-c3cc(-c4cccc5oc6ccccc6c45)c4ccccc4c3)nc(-c3cccc4oc5ccccc5c34)n2)c1 ; c1ccc2c(c1)ccc1cc(-c3nc(-c4cccc5c4oc4ccccc45)nc(-c4cccc5oc6ccccc6c45)n3)ccc12 ; c1ccc2c(c1)ccc1ccc(-c3nc(-c4ccc(-c5cccc6oc7ccccc7c56)cc4)nc(-c4cccc5c4oc4ccccc45)n3)cc12]

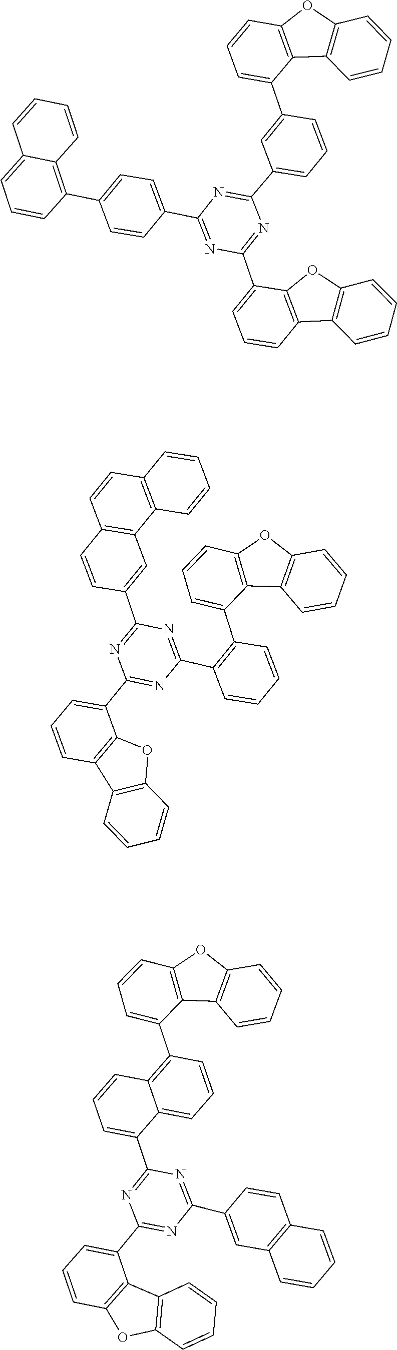 [SMILES: c1cc(-c2nc(-c3ccc(-c4cccc5ccccc45)cc3)nc(-c3cccc4c3oc3ccccc34)n2)cc(-c2cccc3oc4ccccc4c23)c1 ; c1ccc(-c2cccc3oc4ccccc4c23)c(-c2nc(-c3ccc4ccc5ccccc5c4c3)nc(-c3cccc4c3oc3ccccc34)n2)c1 ; c1ccc2cc(-c3nc(-c4cccc5c(-c6cccc7oc8ccccc8c67)cccc45)nc(-c4cccc5oc6ccccc6c45)n3)ccc2c1]

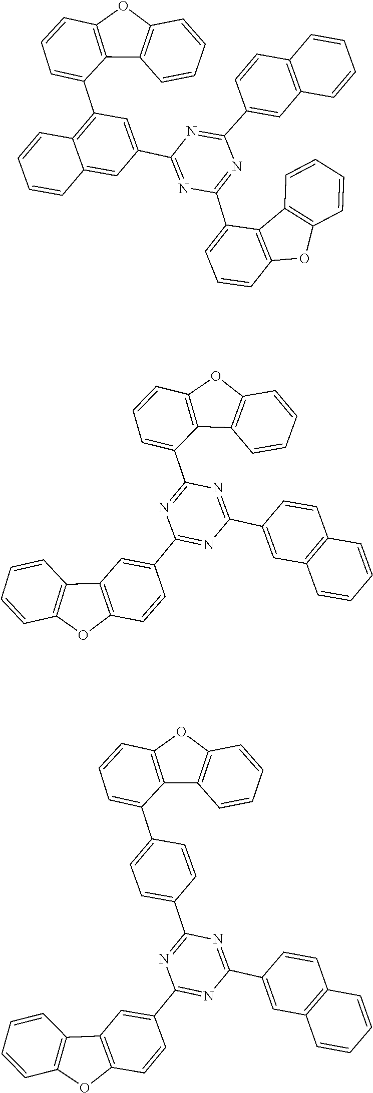 [SMILES: c1ccc2cc(-c3nc(-c4cc(-c5cccc6oc7ccccc7c56)c5ccccc5c4)nc(-c4cccc5oc6ccccc6c45)n3)ccc2c1 ; c1ccc2cc(-c3nc(-c4ccc5oc6ccccc6c5c4)nc(-c4cccc5oc6ccccc6c45)n3)ccc2c1 ; c1ccc2cc(-c3nc(-c4ccc(-c5cccc6oc7ccccc7c56)cc4)nc(-c4ccc5oc6ccccc6c5c4)n3)ccc2c1]

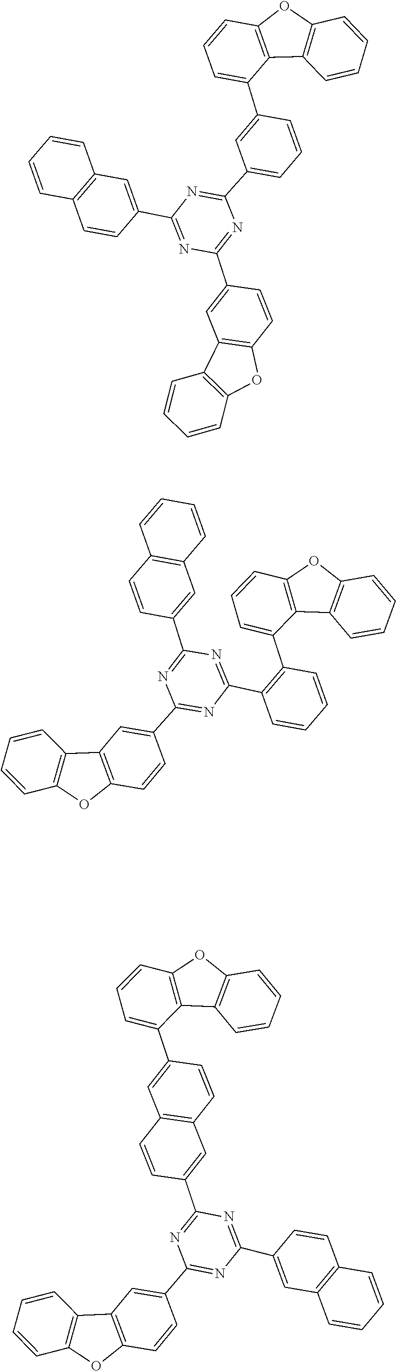 [SMILES: c1cc(-c2nc(-c3ccc4ccccc4c3)nc(-c3ccc4oc5ccccc5c4c3)n2)cc(-c2cccc3oc4ccccc4c23)c1 ; c1ccc(-c2cccc3oc4ccccc4c23)c(-c2nc(-c3ccc4ccccc4c3)nc(-c3ccc4oc5ccccc5c4c3)n2)c1 ; c1ccc2cc(-c3nc(-c4ccc5cc(-c6cccc7oc8ccccc8c67)ccc5c4)nc(-c4ccc5oc6ccccc6c5c4)n3)ccc2c1]

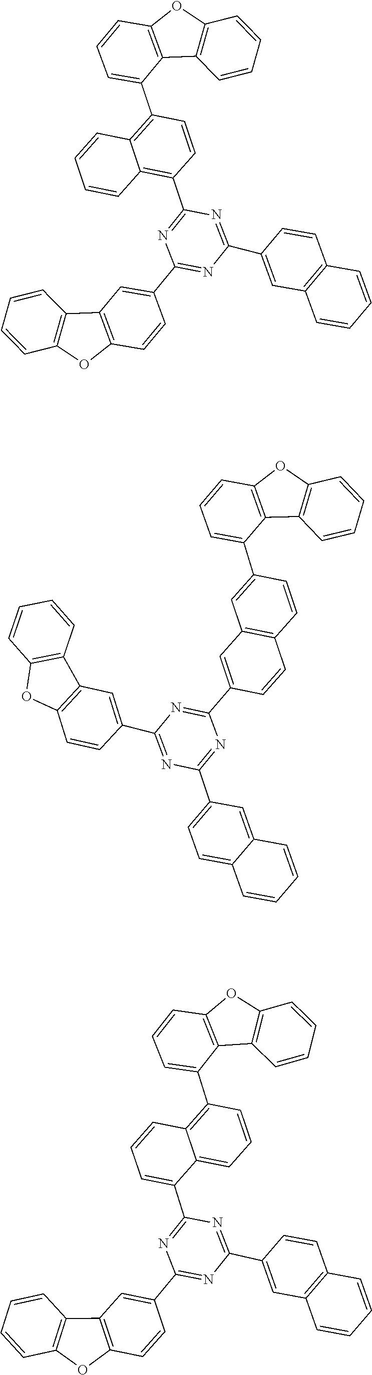 [SMILES: c1ccc2cc(-c3nc(-c4ccc5oc6ccccc6c5c4)nc(-c4ccc(-c5cccc6oc7ccccc7c56)c5ccccc45)n3)ccc2c1 ; c1ccc2cc(-c3nc(-c4ccc5ccc(-c6cccc7oc8ccccc8c67)cc5c4)nc(-c4ccc5oc6ccccc6c5c4)n3)ccc2c1 ; c1ccc2cc(-c3nc(-c4ccc5oc6ccccc6c5c4)nc(-c4cccc5c(-c6cccc7oc8ccccc8c67)cccc45)n3)ccc2c1]

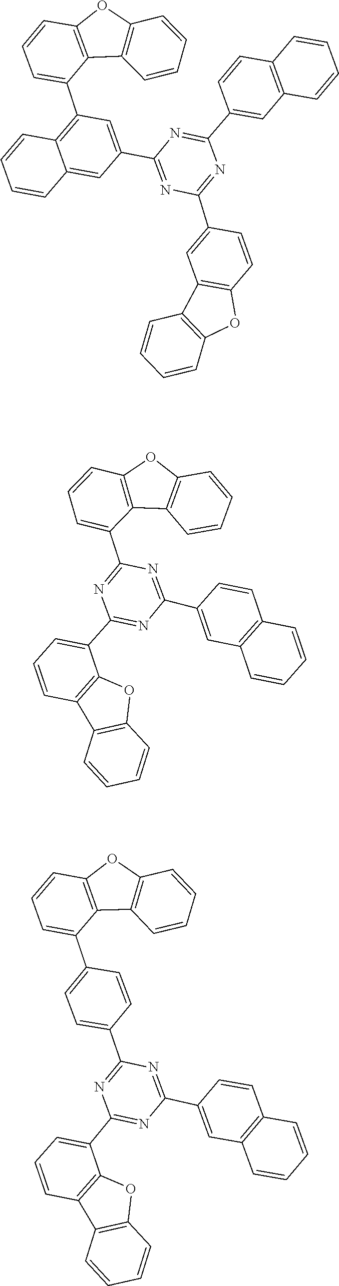 [SMILES: c1ccc2cc(-c3nc(-c4cc(-c5cccc6oc7ccccc7c56)c5ccccc5c4)nc(-c4ccc5oc6ccccc6c5c4)n3)ccc2c1 ; c1ccc2cc(-c3nc(-c4cccc5c4oc4ccccc45)nc(-c4cccc5oc6ccccc6c45)n3)ccc2c1 ; c1ccc2cc(-c3nc(-c4ccc(-c5cccc6oc7ccccc7c56)cc4)nc(-c4cccc5c4oc4ccccc45)n3)ccc2c1]

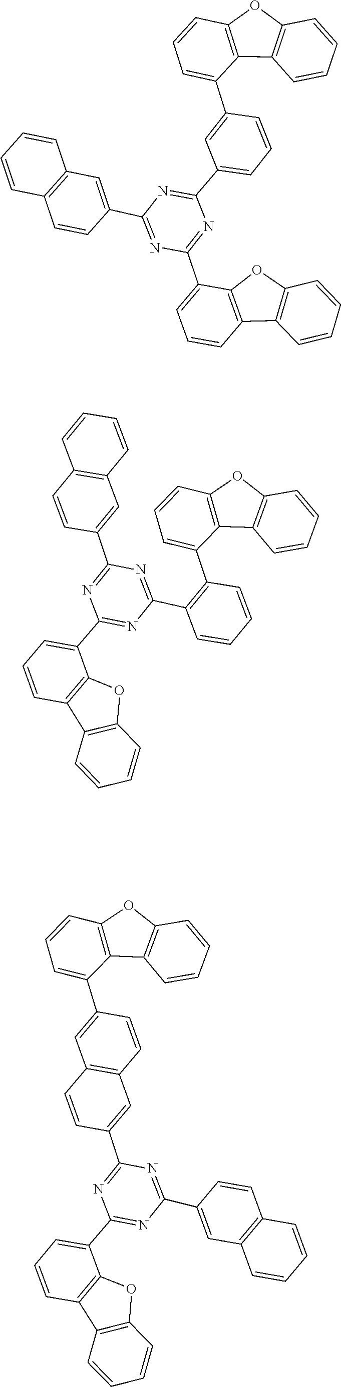 [SMILES: c1cc(-c2nc(-c3ccc4ccccc4c3)nc(-c3cccc4c3oc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3cccc4c3oc3ccccc34)n2)c1 ; c1ccc2cc(-c3nc(-c4ccc5cc(-c6cccc7oc8ccccc8c67)ccc5c4)nc(-c4cccc5c4oc4ccccc45)n3)ccc2c1]

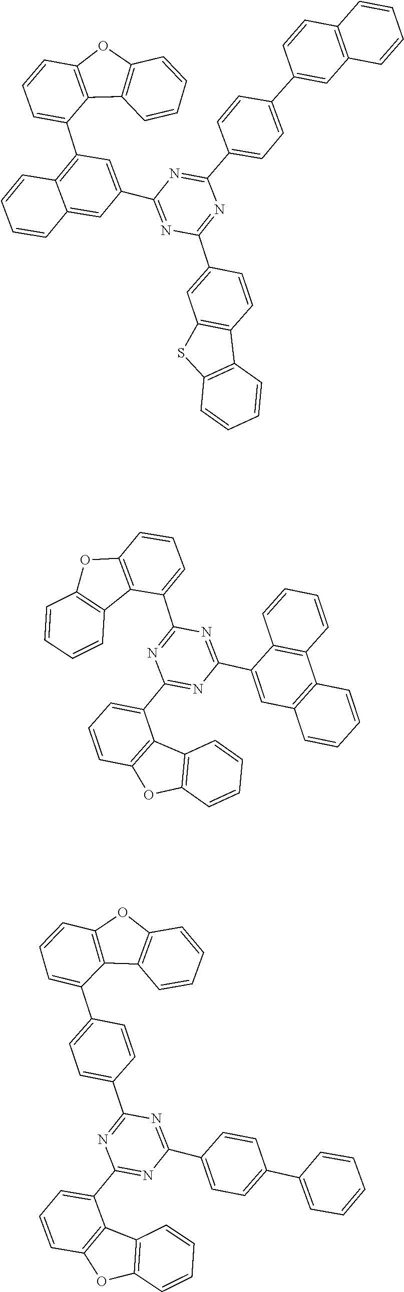 [SMILES: c1ccc2cc(-c3ccc(-c4nc(-c5cc(-c6cccc7oc8ccccc8c67)c6ccccc6c5)nc(-c5ccc6c(c5)sc5ccccc56)n4)cc3)ccc2c1 ; c1ccc2c(c1)cc(-c1nc(-c3cccc4oc5ccccc5c34)nc(-c3cccc4oc5ccccc5c34)n1)c1ccccc12 ; c1ccc(-c2ccc(-c3nc(-c4ccc(-c5cccc6oc7ccccc7c56)cc4)nc(-c4cccc5oc6ccccc6c45)n3)cc2)cc1]

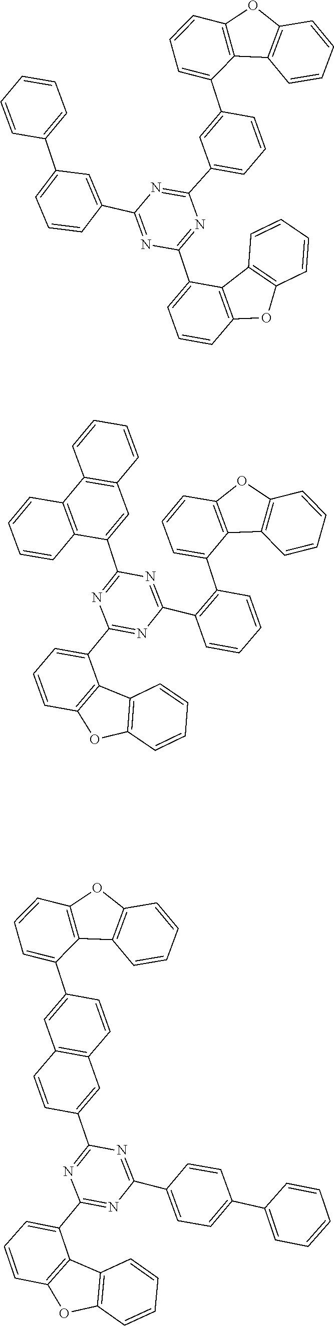 [SMILES: c1ccc(-c2cccc(-c3nc(-c4cccc(-c5cccc6oc7ccccc7c56)c4)nc(-c4cccc5oc6ccccc6c45)n3)c2)cc1 ; c1ccc(-c2cccc3oc4ccccc4c23)c(-c2nc(-c3cc4ccccc4c4ccccc34)nc(-c3cccc4oc5ccccc5c34)n2)c1 ; c1ccc(-c2ccc(-c3nc(-c4ccc5cc(-c6cccc7oc8ccccc8c67)ccc5c4)nc(-c4cccc5oc6ccccc6c45)n3)cc2)cc1]

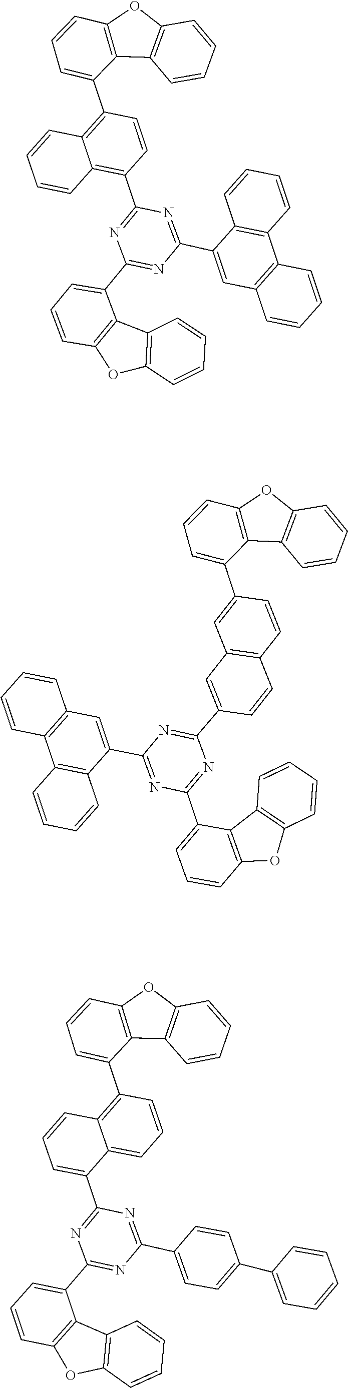 [SMILES: c1ccc2c(c1)cc(-c1nc(-c3ccc(-c4cccc5oc6ccccc6c45)c4ccccc34)nc(-c3cccc4oc5ccccc5c34)n1)c1ccccc12 ; c1ccc2c(c1)cc(-c1nc(-c3ccc4ccc(-c5cccc6oc7ccccc7c56)cc4c3)nc(-c3cccc4oc5ccccc5c34)n1)c1ccccc12 ; c1ccc(-c2ccc(-c3nc(-c4cccc5c(-c6cccc7oc8ccccc8c67)cccc45)nc(-c4cccc5oc6ccccc6c45)n3)cc2)cc1]

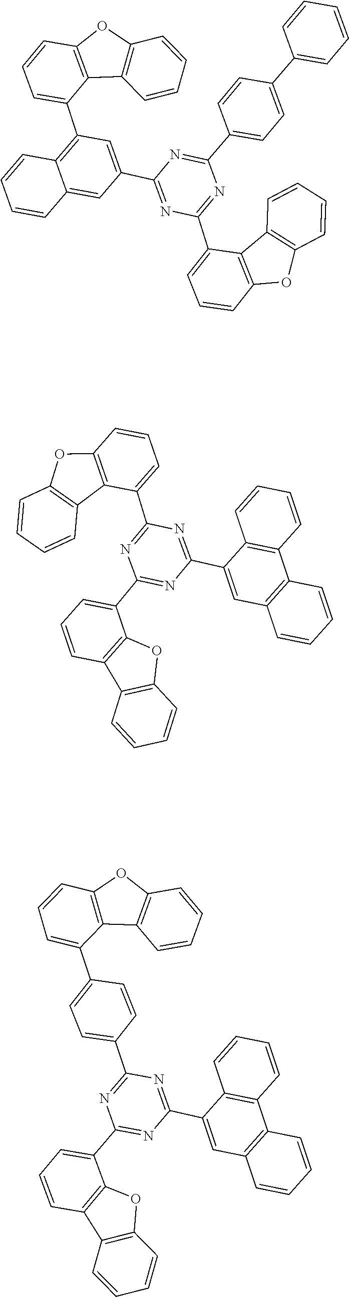 [SMILES: c1ccc(-c2ccc(-c3nc(-c4cc(-c5cccc6oc7ccccc7c56)c5ccccc5c4)nc(-c4cccc5oc6ccccc6c45)n3)cc2)cc1 ; c1ccc2c(c1)cc(-c1nc(-c3cccc4c3oc3ccccc34)nc(-c3cccc4oc5ccccc5c34)n1)c1ccccc12 ; c1ccc2c(c1)cc(-c1nc(-c3ccc(-c4cccc5oc6ccccc6c45)cc3)nc(-c3cccc4c3oc3ccccc34)n1)c1ccccc12]

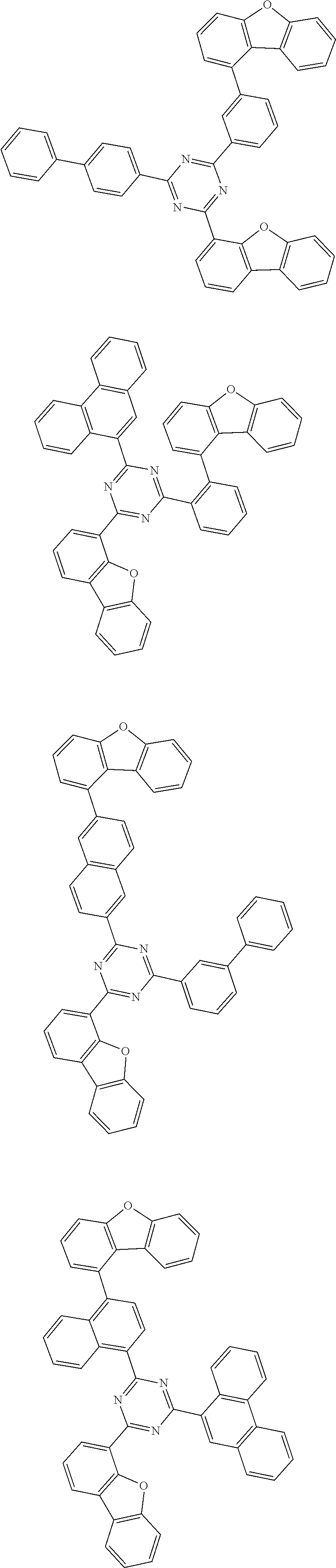 [SMILES: c1ccc(-c2ccc(-c3nc(-c4cccc(-c5cccc6oc7ccccc7c56)c4)nc(-c4cccc5c4oc4ccccc45)n3)cc2)cc1 ; c1ccc(-c2cccc3oc4ccccc4c23)c(-c2nc(-c3cc4ccccc4c4ccccc34)nc(-c3cccc4c3oc3ccccc34)n2)c1 ; c1ccc(-c2cccc(-c3nc(-c4ccc5cc(-c6cccc7oc8ccccc8c67)ccc5c4)nc(-c4cccc5c4oc4ccccc45)n3)c2)cc1 ; c1ccc2c(c1)cc(-c1nc(-c3ccc(-c4cccc5oc6ccccc6c45)c4ccccc34)nc(-c3cccc4c3oc3ccccc34)n1)c1ccccc12]

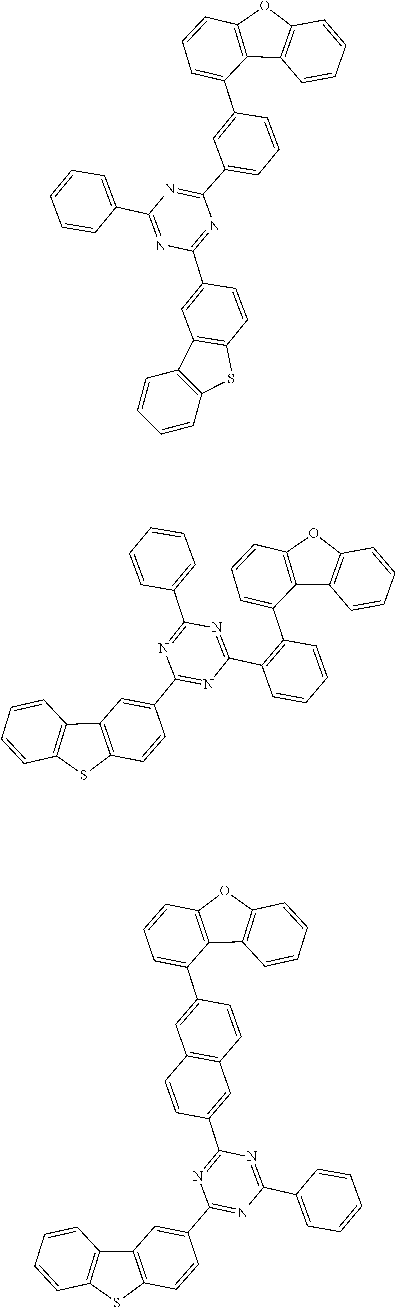 [SMILES: c1ccc(-c2nc(-c3cccc(-c4cccc5oc6ccccc6c45)c3)nc(-c3ccc4sc5ccccc5c4c3)n2)cc1 ; c1ccc(-c2nc(-c3ccc4sc5ccccc5c4c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4sc5ccccc5c4c3)n2)cc1]

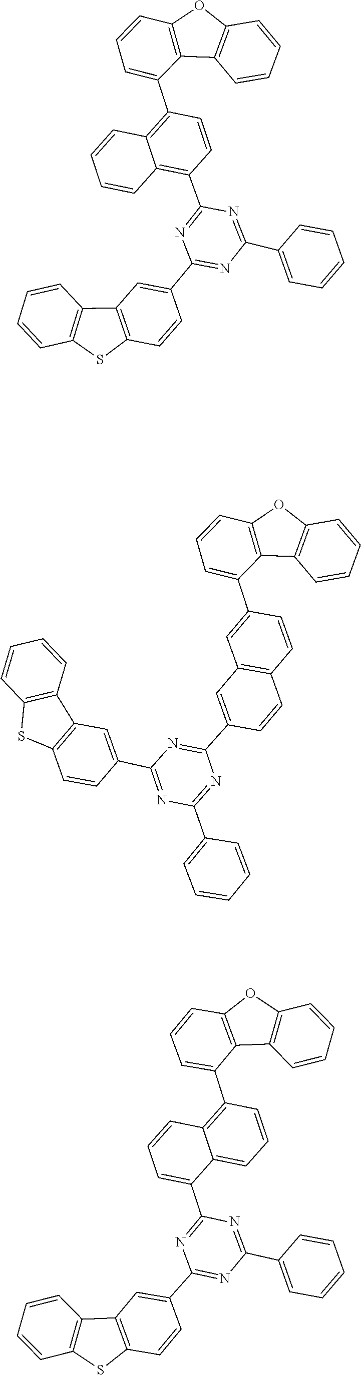 [SMILES: c1ccc(-c2nc(-c3ccc4sc5ccccc5c4c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4sc5ccccc5c4c3)n2)cc1 ; c1ccc(-c2nc(-c3ccc4sc5ccccc5c4c3)nc(-c3cccc4c(-c5cccc6oc7ccccc7c56)cccc34)n2)cc1]

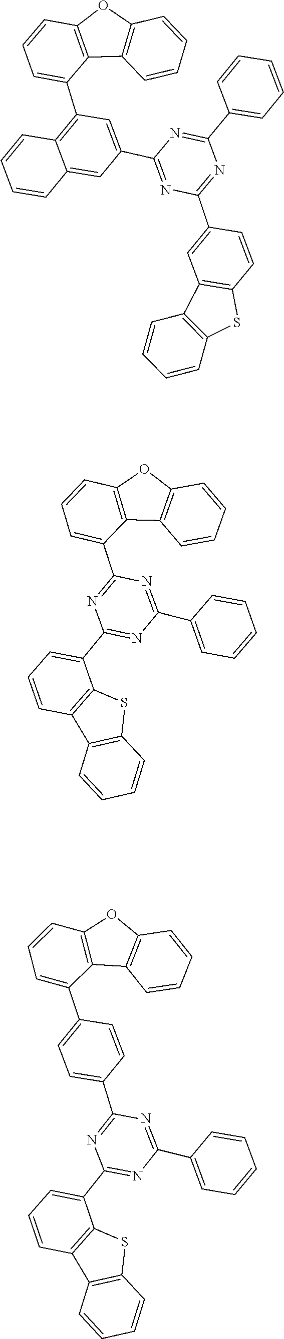 [SMILES: c1ccc(-c2nc(-c3cc(-c4cccc5oc6ccccc6c45)c4ccccc4c3)nc(-c3ccc4sc5ccccc5c4c3)n2)cc1 ; c1ccc(-c2nc(-c3cccc4c3sc3ccccc34)nc(-c3cccc4oc5ccccc5c34)n2)cc1 ; c1ccc(-c2nc(-c3ccc(-c4cccc5oc6ccccc6c45)cc3)nc(-c3cccc4c3sc3ccccc34)n2)cc1]

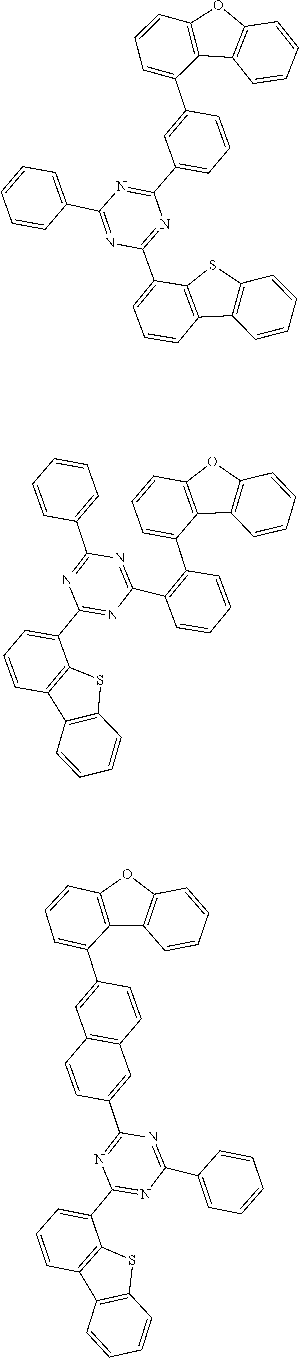 [SMILES: c1ccc(-c2nc(-c3cccc(-c4cccc5oc6ccccc6c45)c3)nc(-c3cccc4c3sc3ccccc34)n2)cc1 ; c1ccc(-c2nc(-c3ccccc3-c3cccc4oc5ccccc5c34)nc(-c3cccc4c3sc3ccccc34)n2)cc1 ; c1ccc(-c2nc(-c3ccc4cc(-c5cccc6oc7ccccc7c56)ccc4c3)nc(-c3cccc4c3sc3ccccc34)n2)cc1]

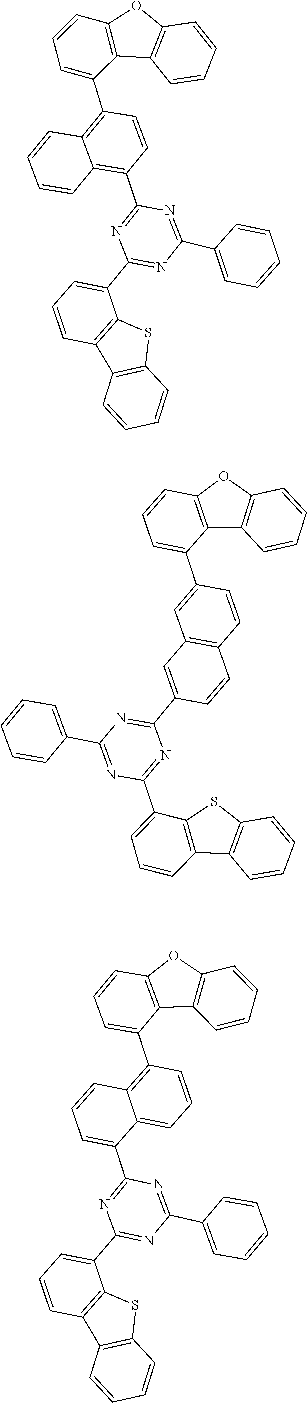 [SMILES: c1ccc(-c2nc(-c3ccc(-c4cccc5oc6ccccc6c45)c4ccccc34)nc(-c3cccc4c3sc3ccccc34)n2)cc1 ; c1ccc(-c2nc(-c3ccc4ccc(-c5cccc6oc7ccccc7c56)cc4c3)nc(-c3cccc4c3sc3ccccc34)n2)cc1 ; c1ccc(-c2nc(-c3cccc4c(-c5cccc6oc7ccccc7c56)cccc34)nc(-c3cccc4c3sc3ccccc34)n2)cc1]

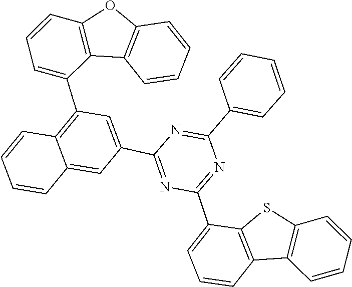 [SMILES: c1ccc(-c2nc(-c3cc(-c4cccc5oc6ccccc6c45)c4ccccc4c3)nc(-c3cccc4c3sc3ccccc34)n2)cc1]

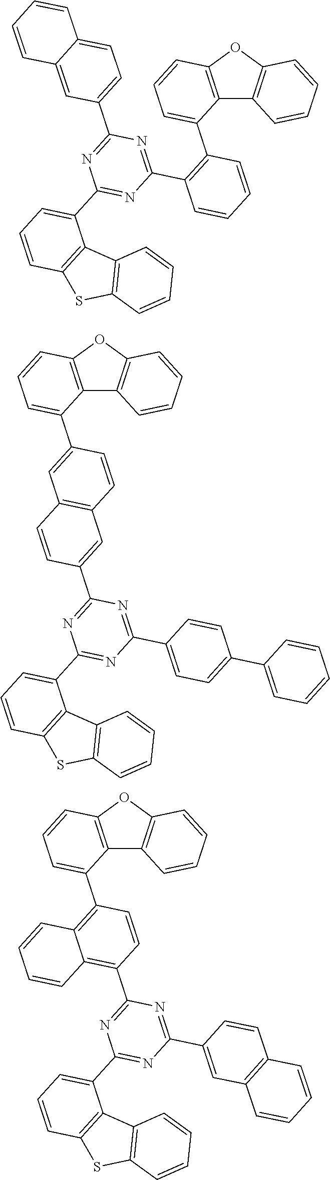

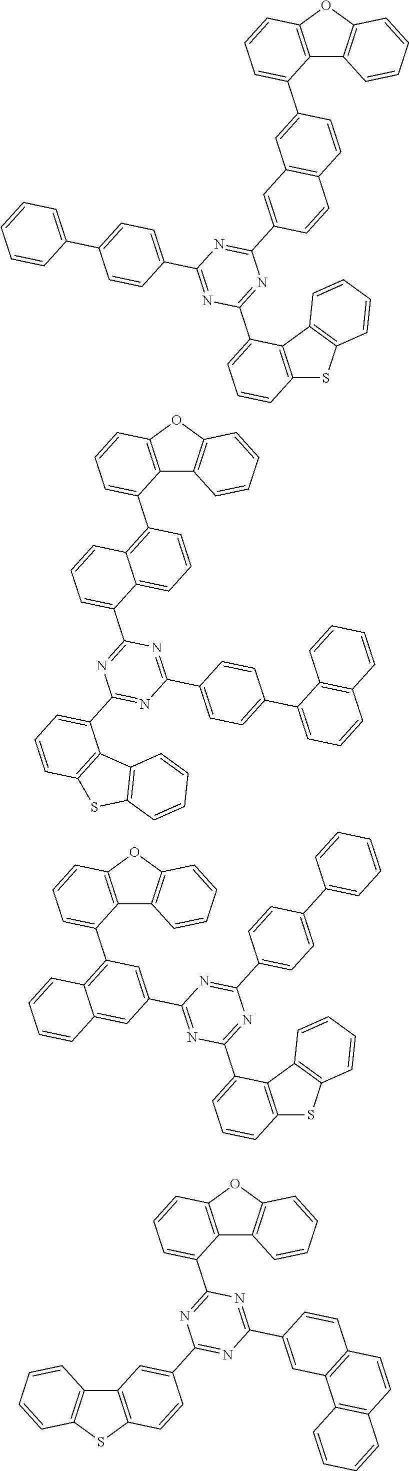

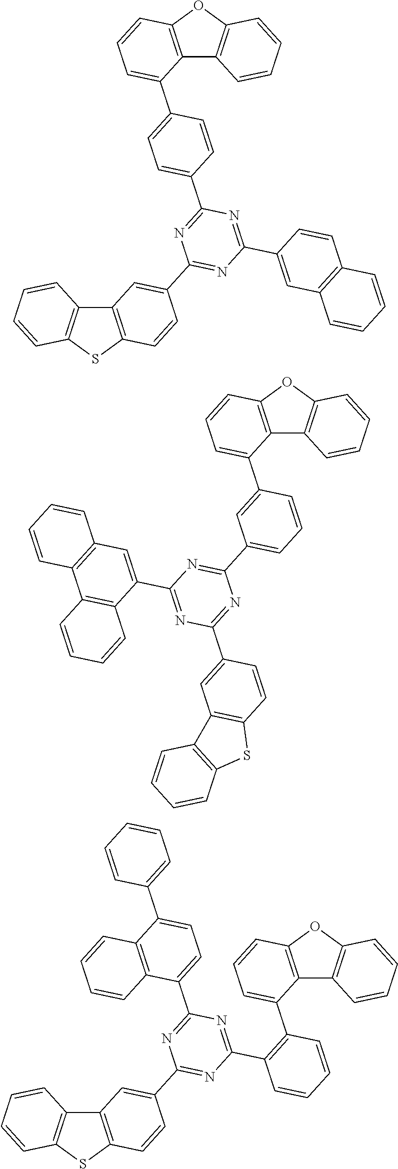

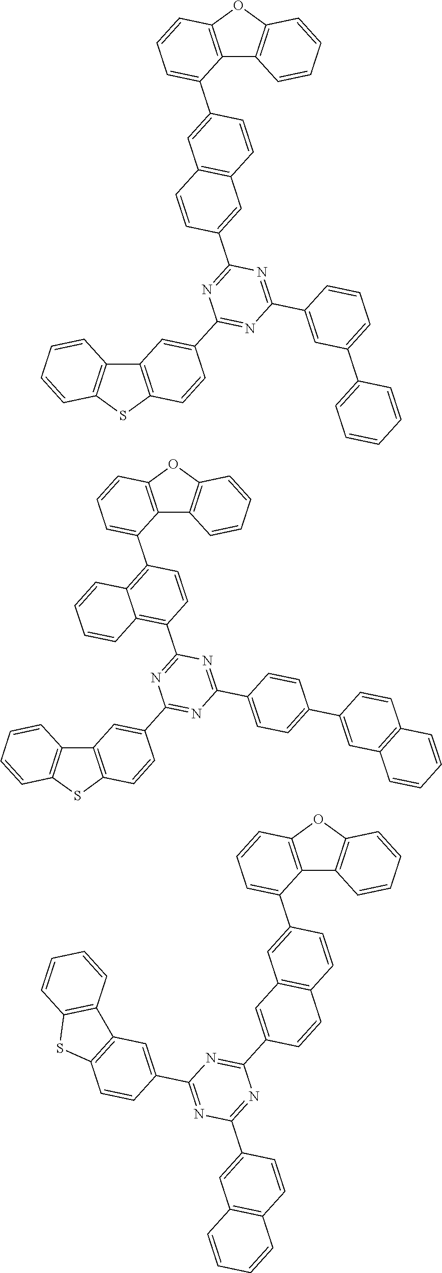 [SMILES: c1ccc(-c2cccc(-c3nc(-c4ccc5cc(-c6cccc7oc8ccccc8c67)ccc5c4)nc(-c4ccc5sc6ccccc6c5c4)n3)c2)cc1 ; c1ccc2cc(-c3ccc(-c4nc(-c5ccc6sc7ccccc7c6c5)nc(-c5ccc(-c6cccc7oc8ccccc8c67)c6ccccc56)n4)cc3)ccc2c1 ; c1ccc2cc(-c3nc(-c4ccc5ccc(-c6cccc7oc8ccccc8c67)cc5c4)nc(-c4ccc5sc6ccccc6c5c4)n3)ccc2c1]

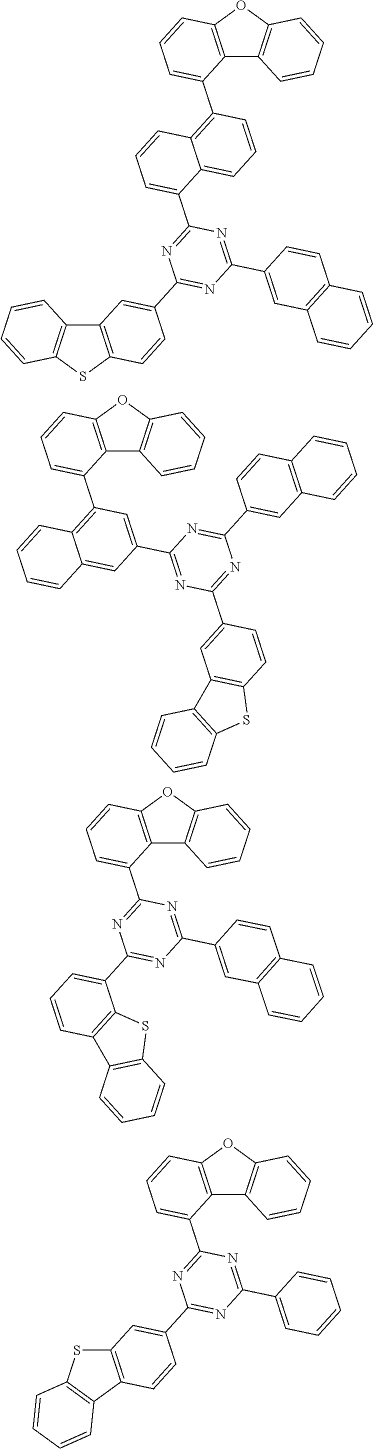

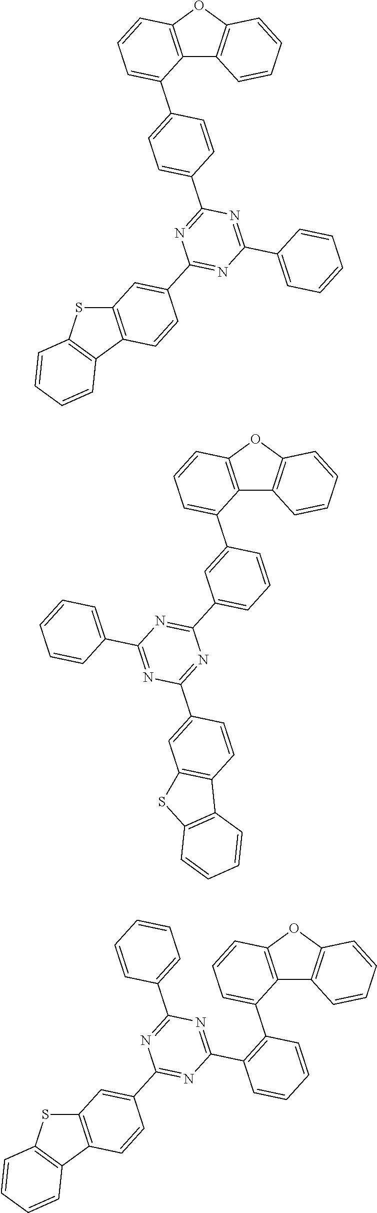 [SMILES: c1ccc(-c2nc(-c3ccc(-c4cccc5oc6ccccc6c45)cc3)nc(-c3ccc4c(c3)sc3ccccc34)n2)cc1 ; c1ccc(-c2nc(-c3cccc(-c4cccc5oc6ccccc6c45)c3)nc(-c3ccc4c(c3)sc3ccccc34)n2)cc1 ; c1ccc(-c2nc(-c3ccc4c(c3)sc3ccccc34)nc(-c3ccccc3-c3cccc4oc5ccccc5c34)n2)cc1]

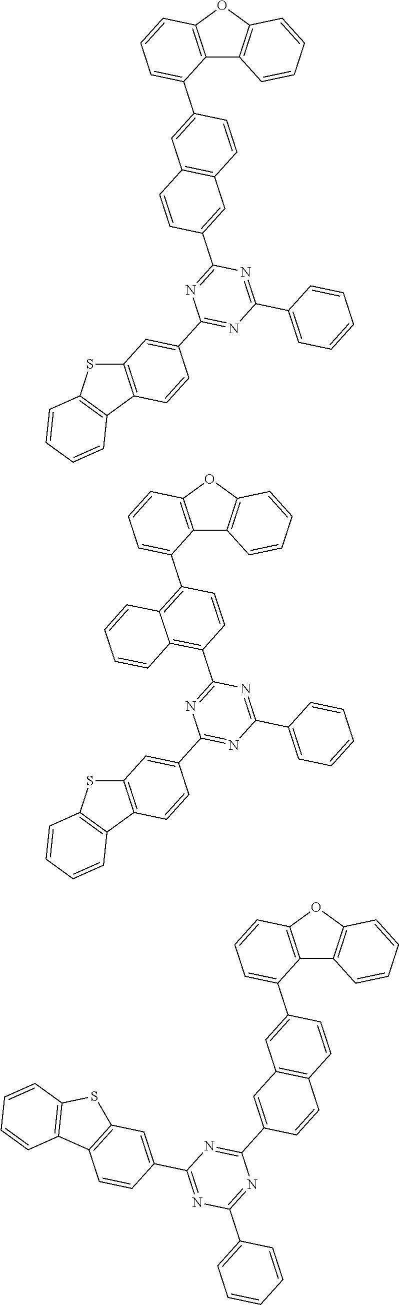 [SMILES: c1ccc(-c2nc(-c3ccc4cc(-c5cccc6oc7ccccc7c56)ccc4c3)nc(-c3ccc4c(c3)sc3ccccc34)n2)cc1 ; c1ccc(-c2nc(-c3ccc4c(c3)sc3ccccc34)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(c3)sc3ccccc34)n2)cc1]

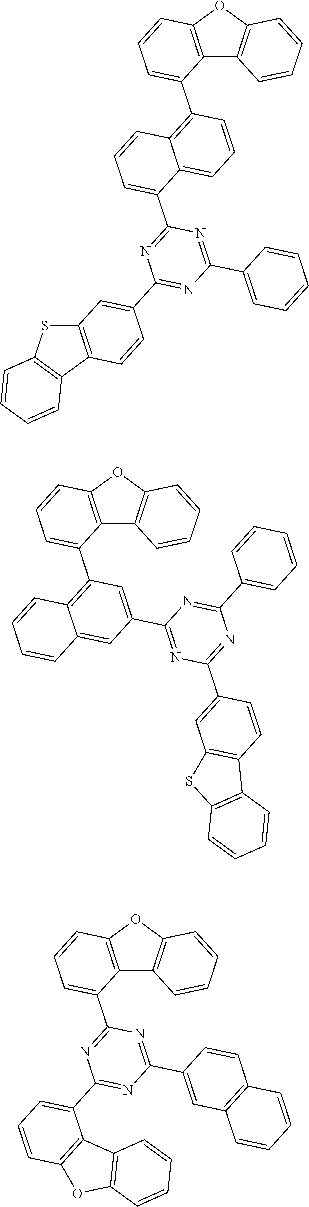 [SMILES: c1ccc(-c2nc(-c3ccc4c(c3)sc3ccccc34)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(c3)sc3ccccc34)n2)cc1 ; c1ccc2cc(-c3nc(-c4cccc5oc6ccccc6c45)nc(-c4cccc5oc6ccccc6c45)n3)ccc2c1]

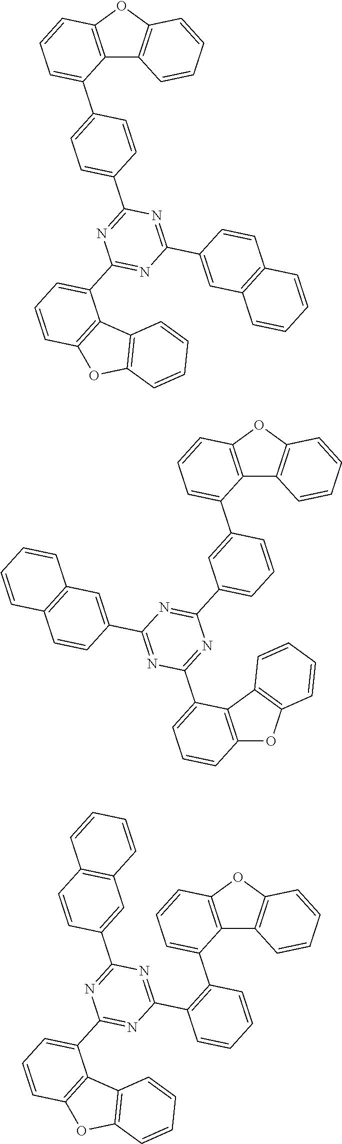 [SMILES: c1ccc2cc(-c3nc(-c4ccc(-c5cccc6oc7ccccc7c56)cc4)nc(-c4cccc5oc6ccccc6c45)n3)ccc2c1 ; c1cc(-c2nc(-c3ccc4ccccc4c3)nc(-c3cccc4oc5ccccc5c34)n2)cc(-c2cccc3oc4ccccc4c23)c1 ; c1ccc(-c2cccc3oc4ccccc4c23)c(-c2nc(-c3ccc4ccccc4c3)nc(-c3cccc4oc5ccccc5c34)n2)c1]

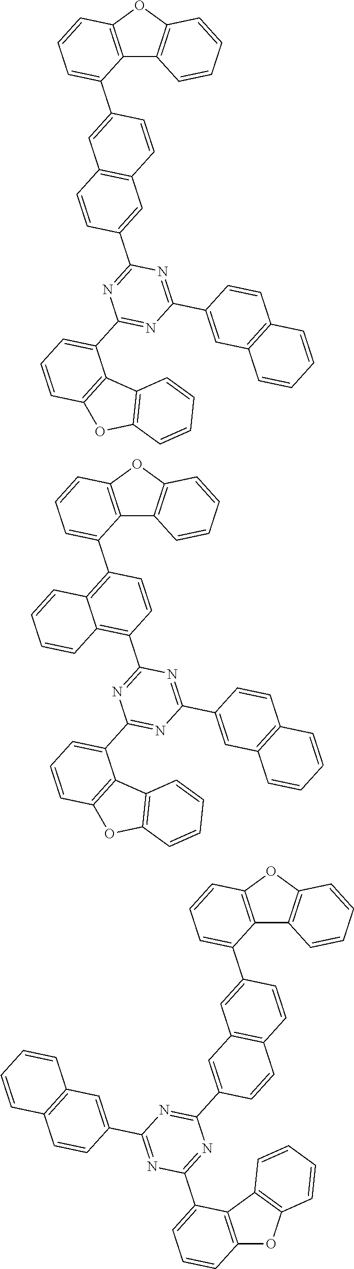

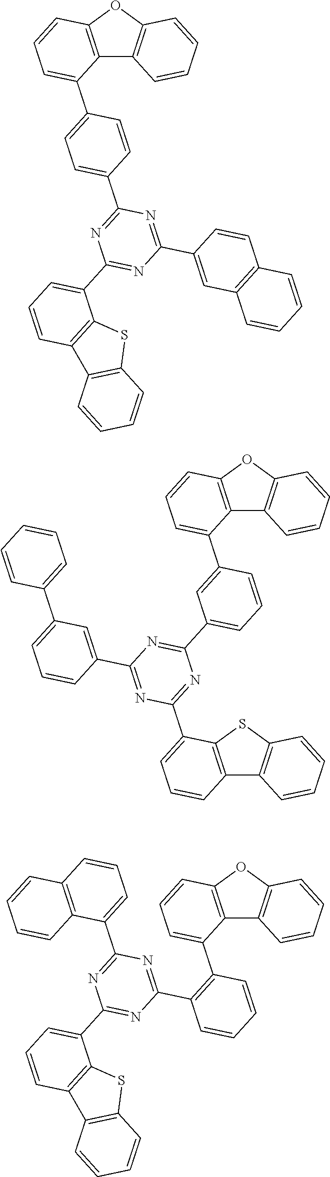 [SMILES: c1ccc2cc(-c3nc(-c4ccc(-c5cccc6oc7ccccc7c56)cc4)nc(-c4cccc5c4sc4ccccc45)n3)ccc2c1 ; c1ccc(-c2cccc(-c3nc(-c4cccc(-c5cccc6oc7ccccc7c56)c4)nc(-c4cccc5c4sc4ccccc45)n3)c2)cc1 ; c1ccc(-c2cccc3oc4ccccc4c23)c(-c2nc(-c3cccc4ccccc34)nc(-c3cccc4c3sc3ccccc34)n2)c1]

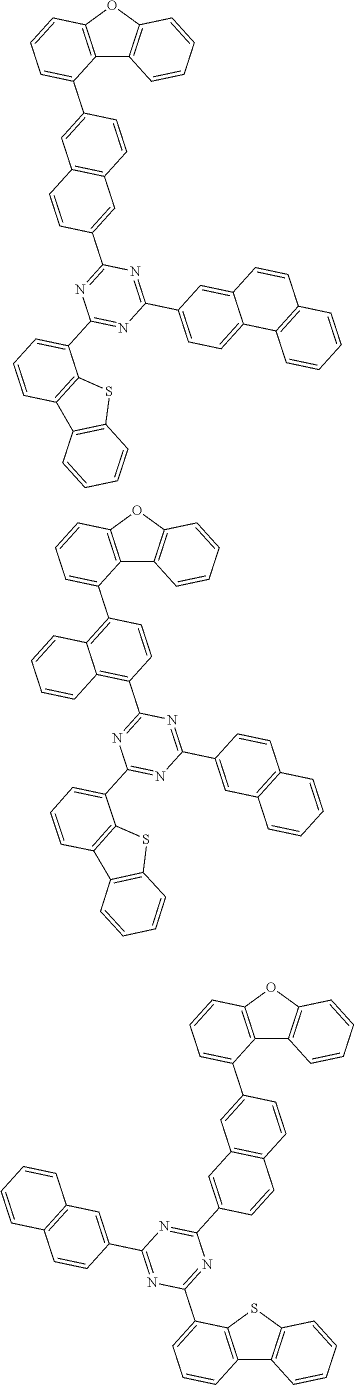

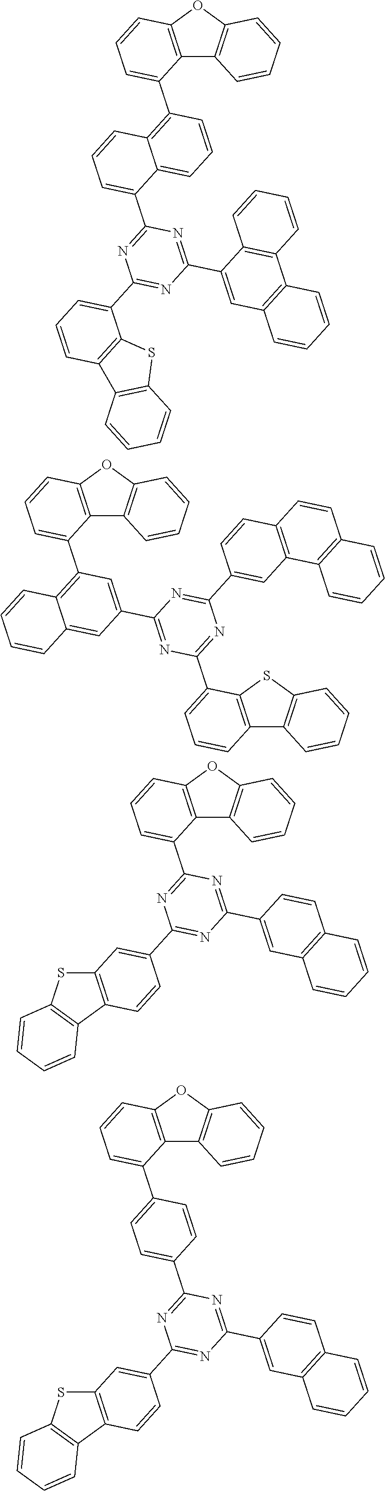

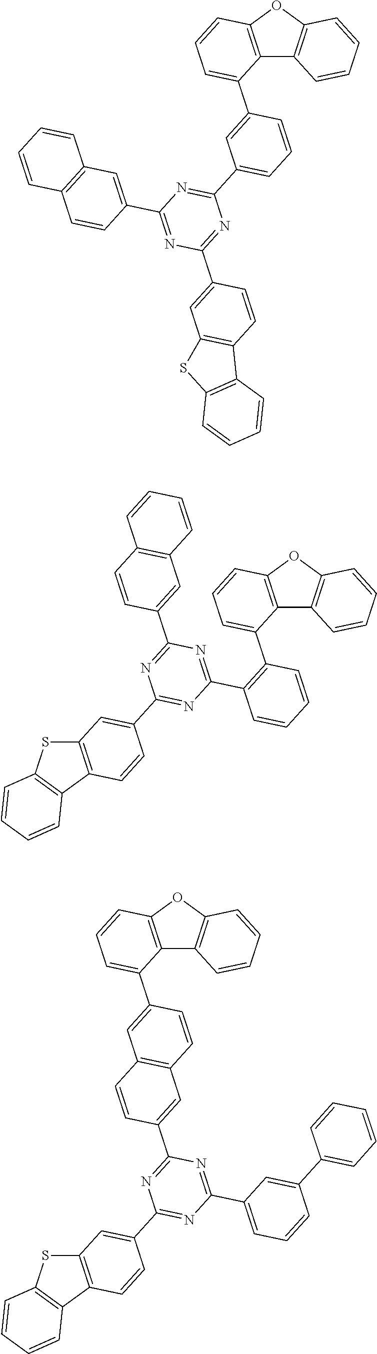 [SMILES: c1cc(-c2nc(-c3ccc4ccccc4c3)nc(-c3ccc4c(c3)sc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3ccc4c(c3)sc3ccccc34)n2)c1 ; c1ccc(-c2cccc(-c3nc(-c4ccc5cc(-c6cccc7oc8ccccc8c67)ccc5c4)nc(-c4ccc5c(c4)sc4ccccc45)n3)c2)cc1]

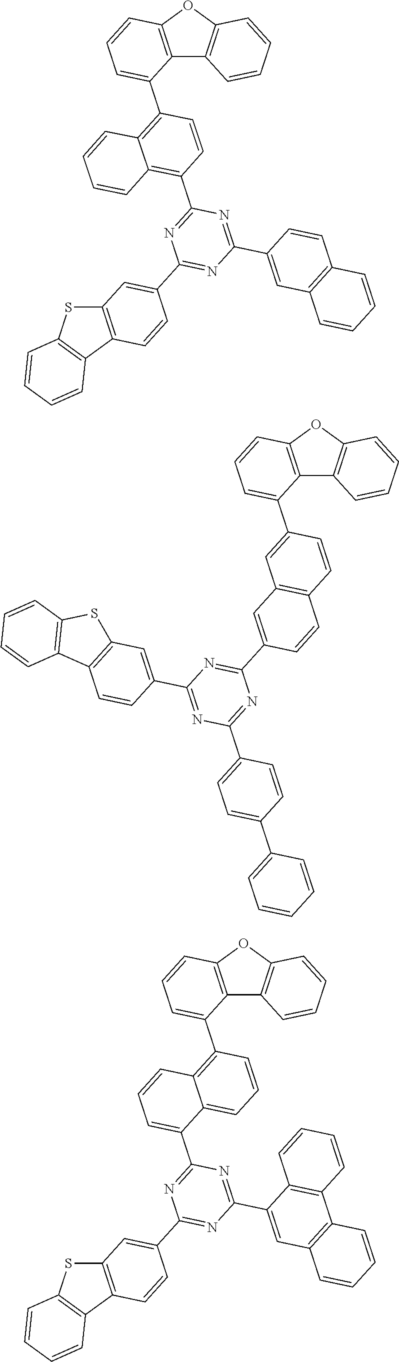

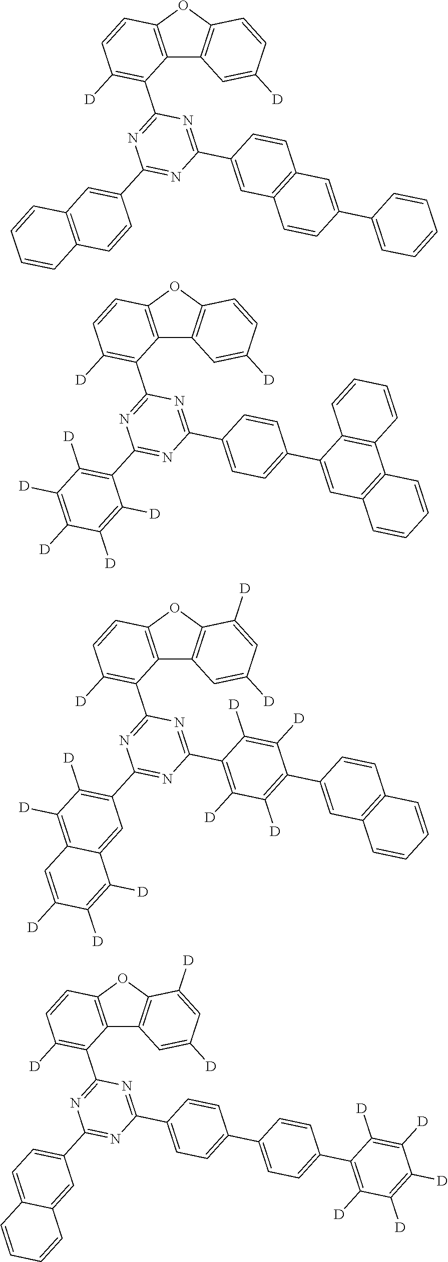

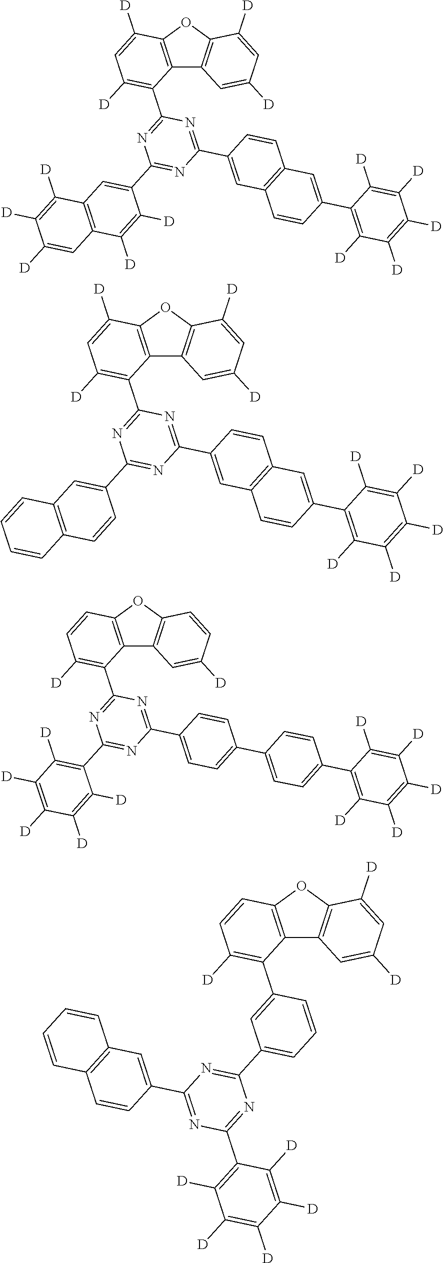

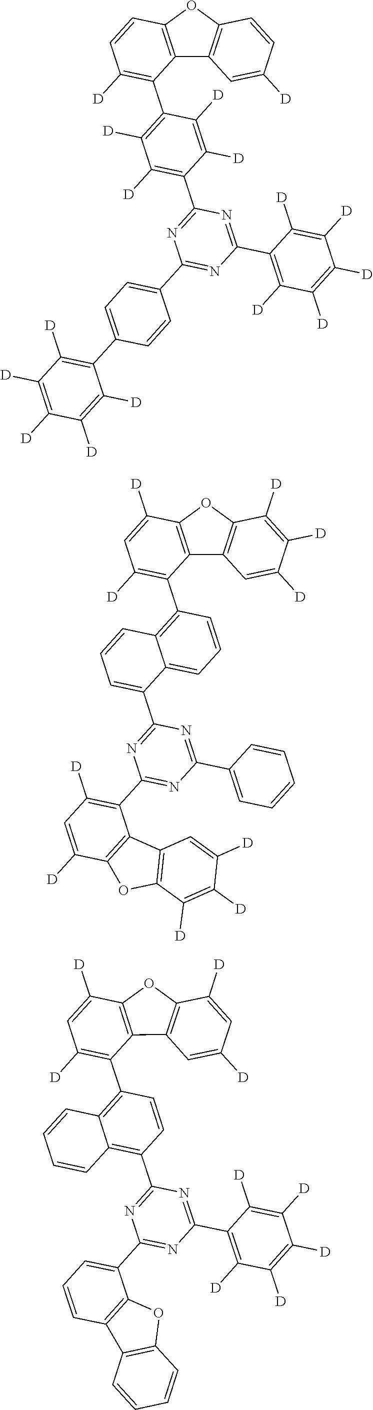 [SMILES: [2H]c1ccc2oc3ccc([2H])c(-c4c([2H])c([2H])c(-c5nc(-c6ccc(-c7c([2H])c([2H])c([2H])c([2H])c7[2H])cc6)nc(-c6c([2H])c([2H])c([2H])c([2H])c6[2H])n5)c([2H])c4[2H])c3c2c1 ; [2H]c1cc2c(oc3c([2H])cc([2H])c(-c4nc(-c5ccccc5)nc(-c5cccc6c(-c7c([2H])cc([2H])c8oc9c([2H])c([2H])c([2H])cc9c78)cccc56)n4)c32)c([2H])c1[2H] ; [2H]c1cc([2H])c2oc3c([2H])cc([2H])c(-c4ccc(-c5nc(-c6c([2H])c([2H])c([2H])c([2H])c6[2H])nc(-c6cccc7c6oc6ccccc67)n5)c5ccccc45)c3c2c1]

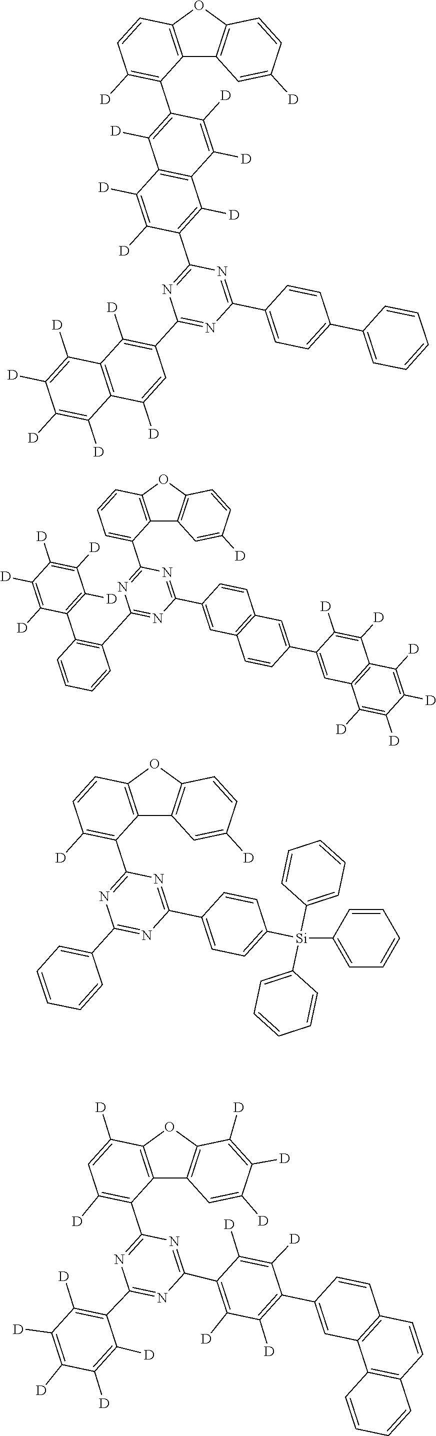

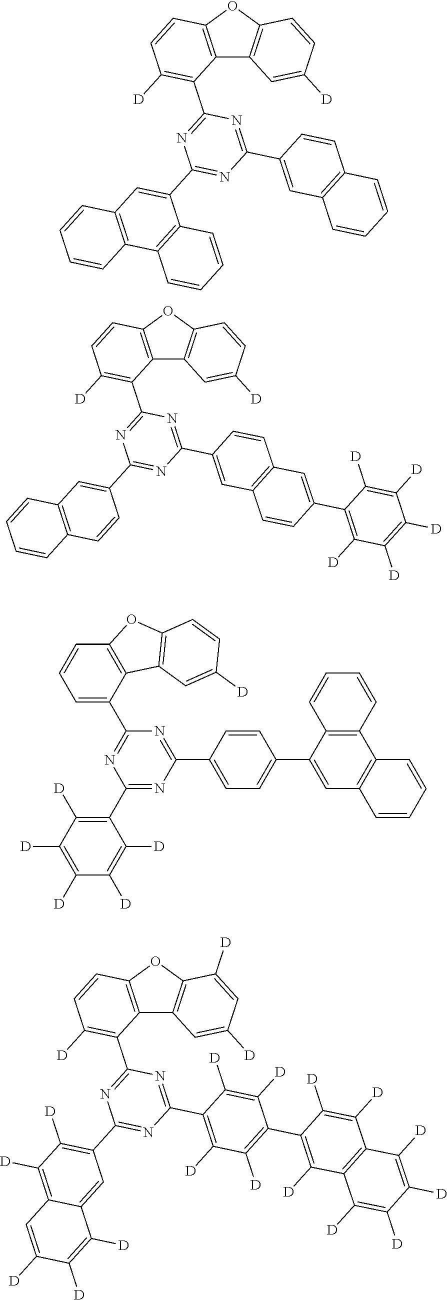

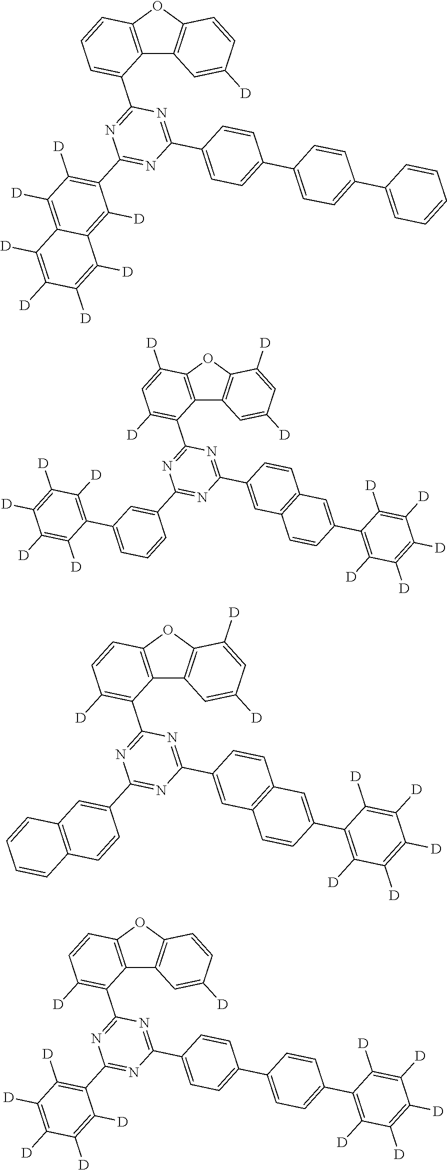

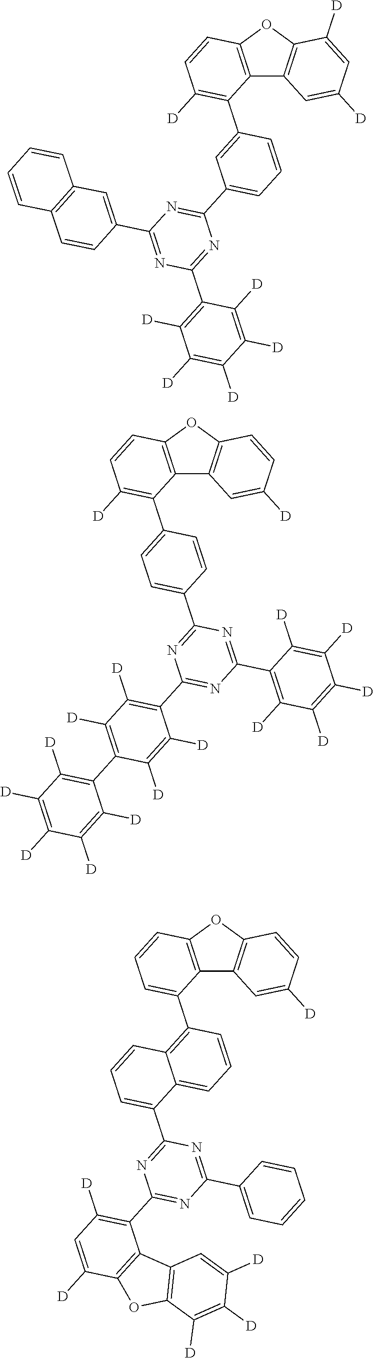

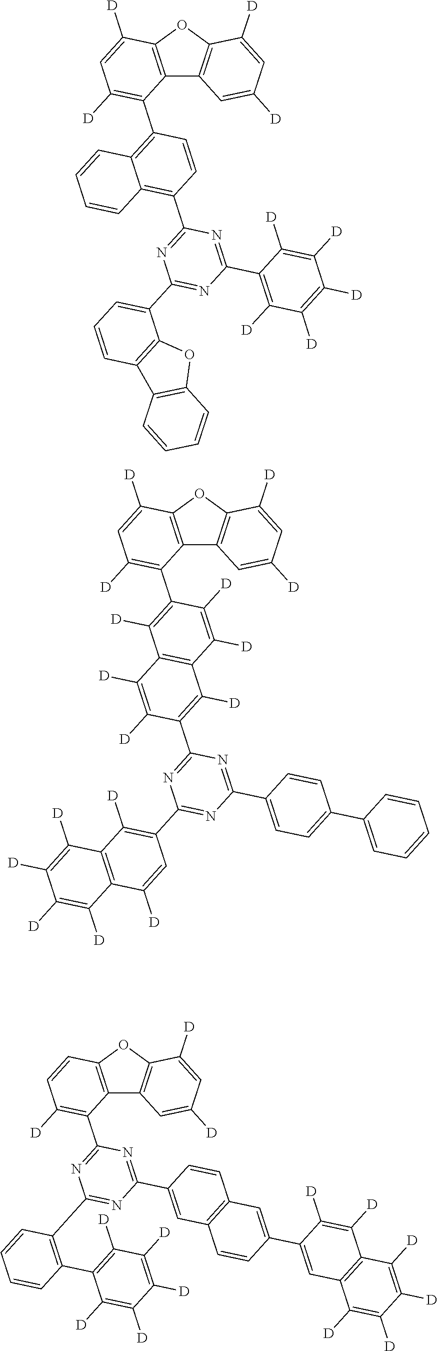

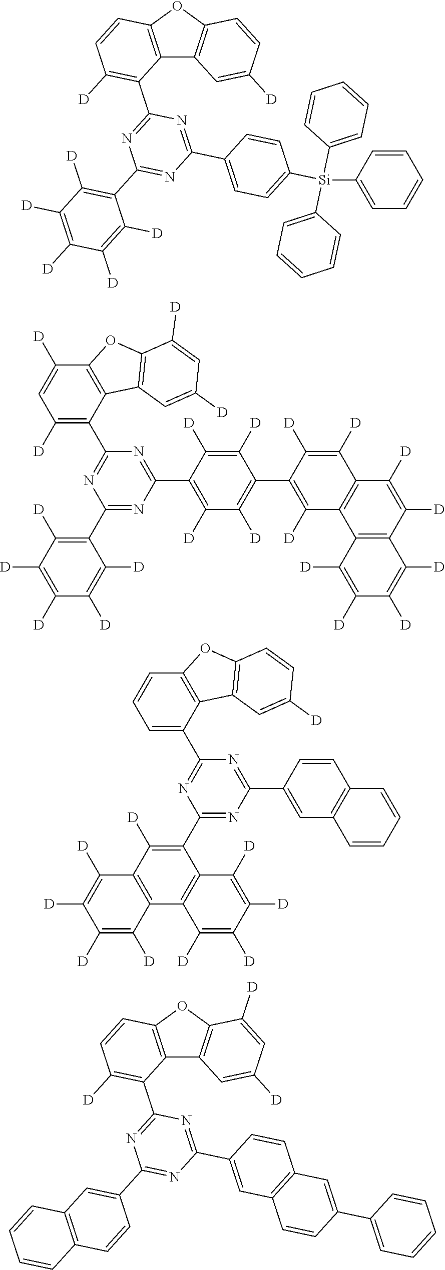

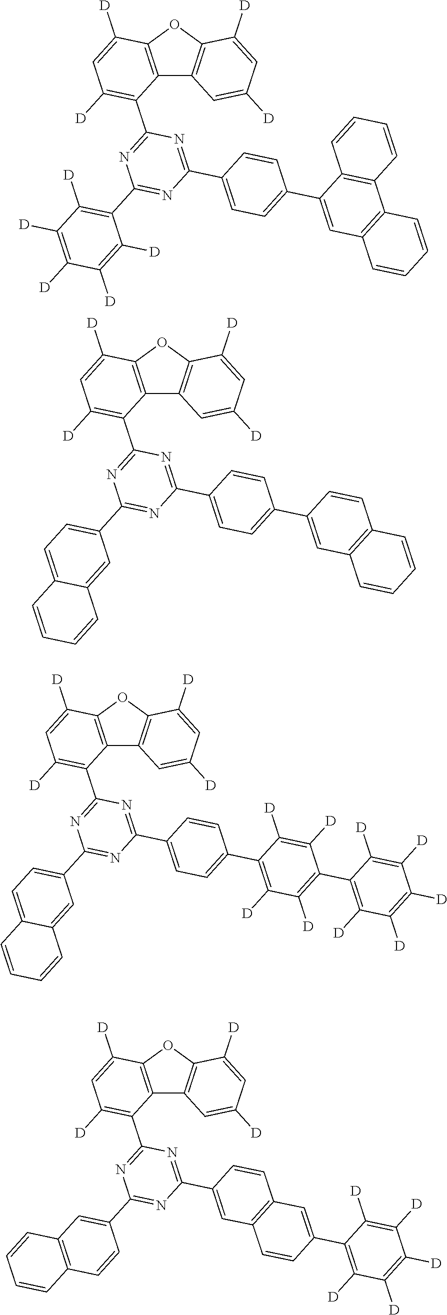

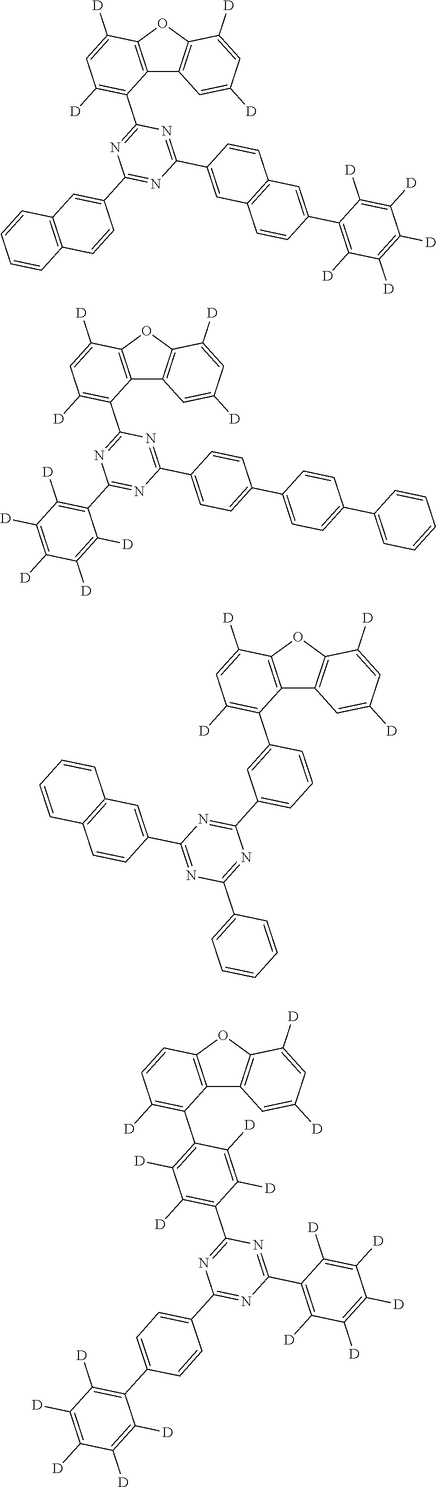

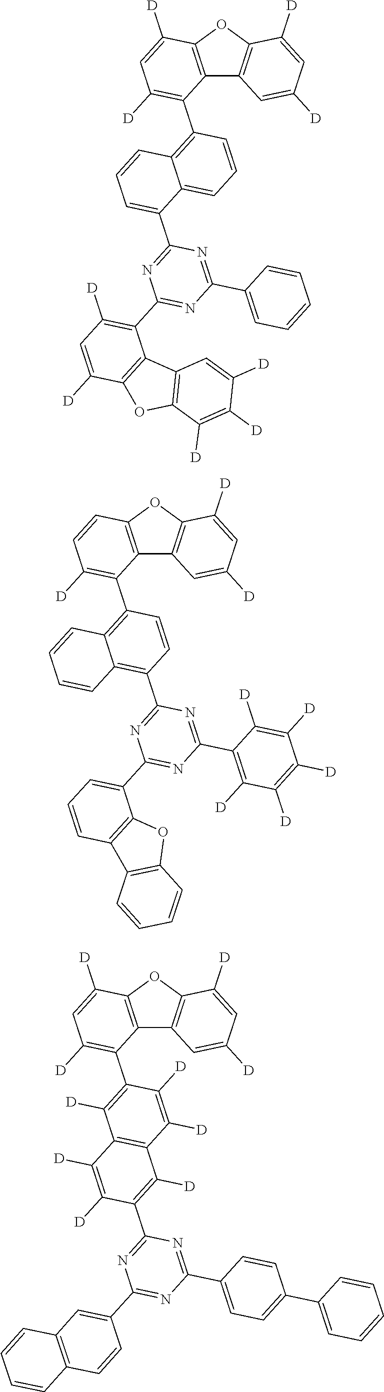

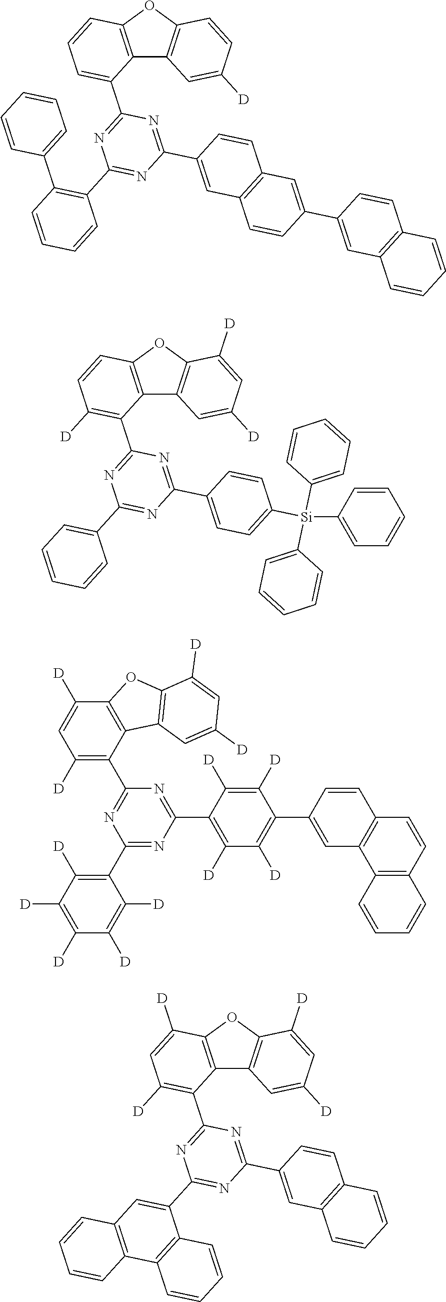 [SMILES: [2H]c1ccc2oc3cccc(-c4nc(-c5ccc6cc(-c7ccc8ccccc8c7)ccc6c5)nc(-c5ccccc5-c5ccccc5)n4)c3c2c1 ; [2H]c1cc([2H])c2oc3ccc([2H])c(-c4nc(-c5ccccc5)nc(-c5ccc([Si](c6ccccc6)(c6ccccc6)c6ccccc6)cc5)n4)c3c2c1 ; [2H]c1cc([2H])c2oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c3c2c1 ; [2H]c1cc([2H])c2oc3c([2H])cc([2H])c(-c4nc(-c5ccc6ccccc6c5)nc(-c5cc6ccccc6c6ccccc56)n4)c3c2c1]

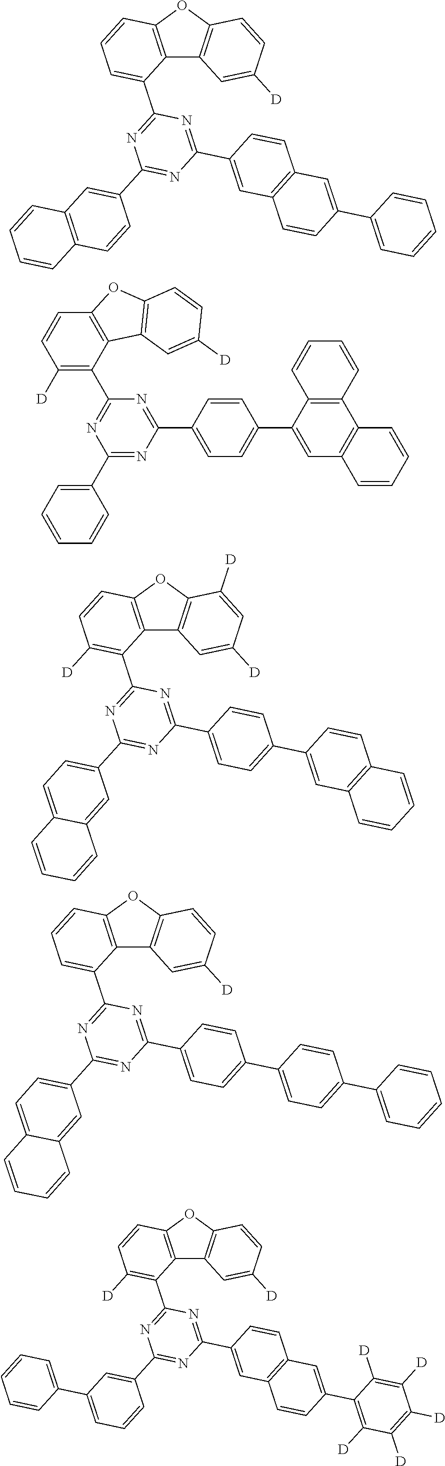

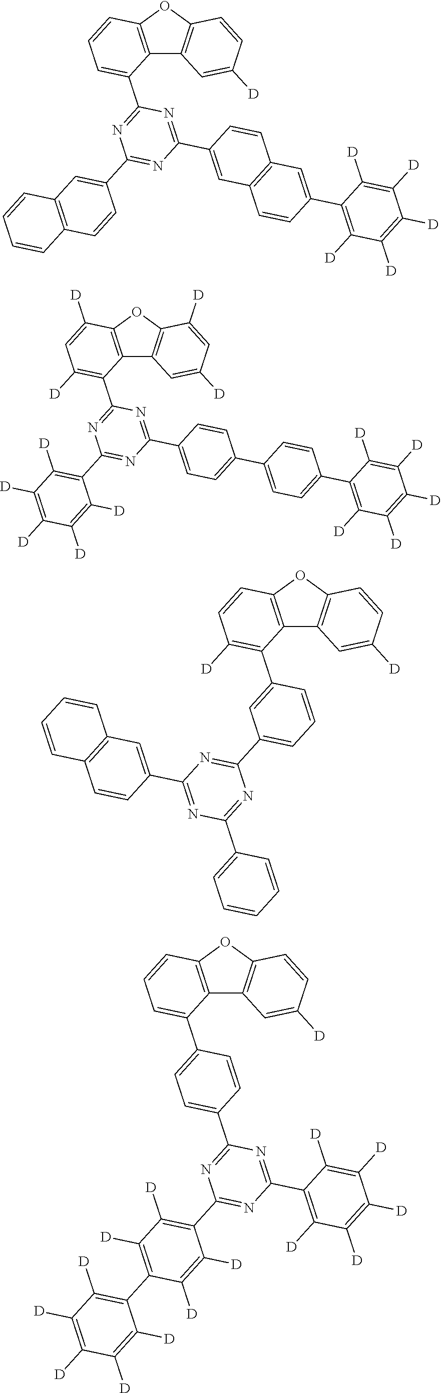

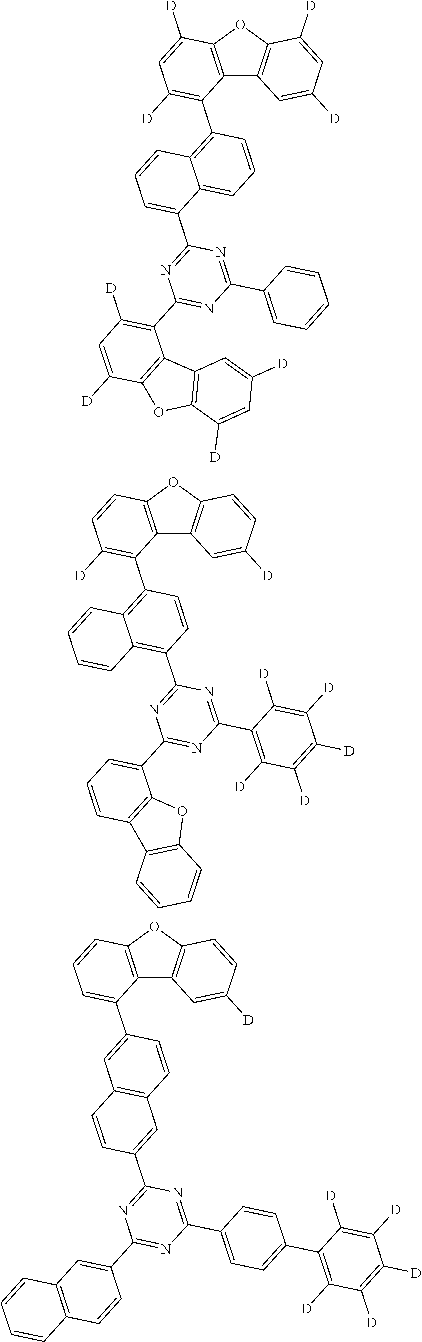

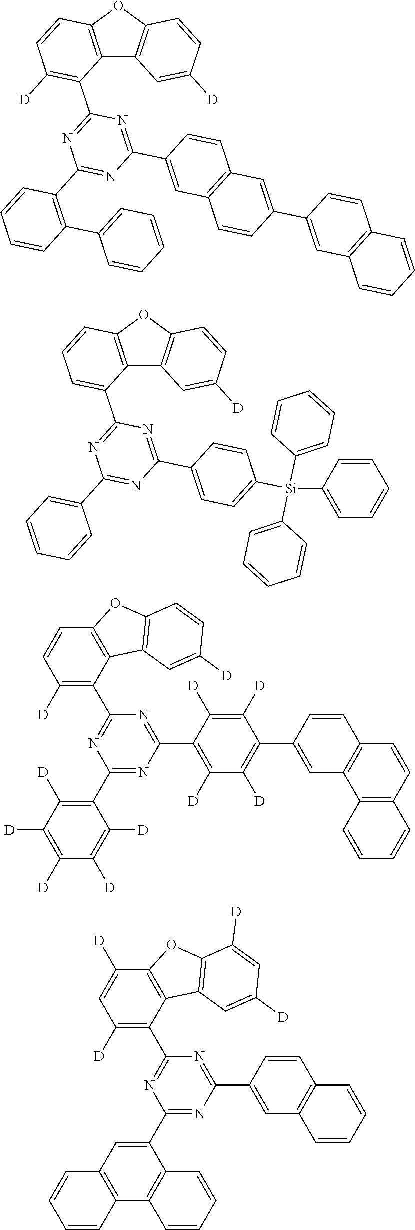

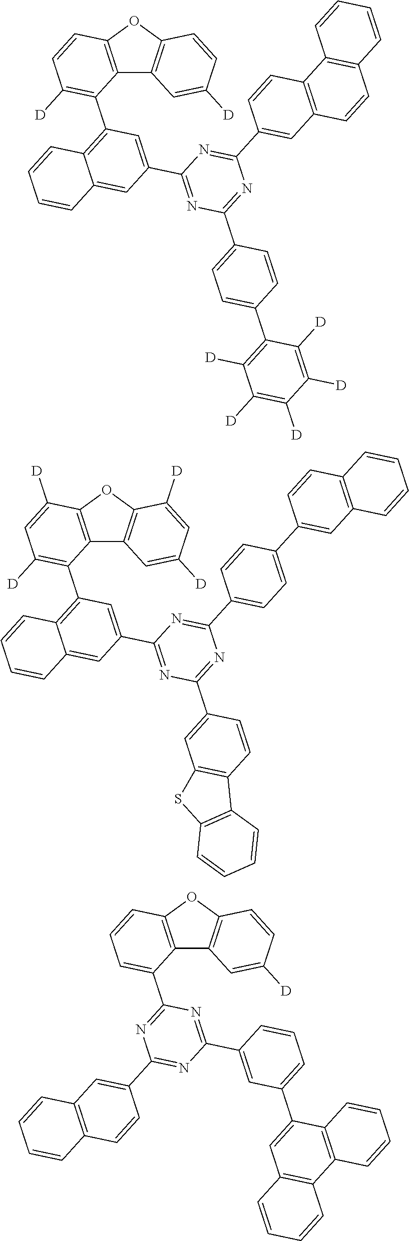

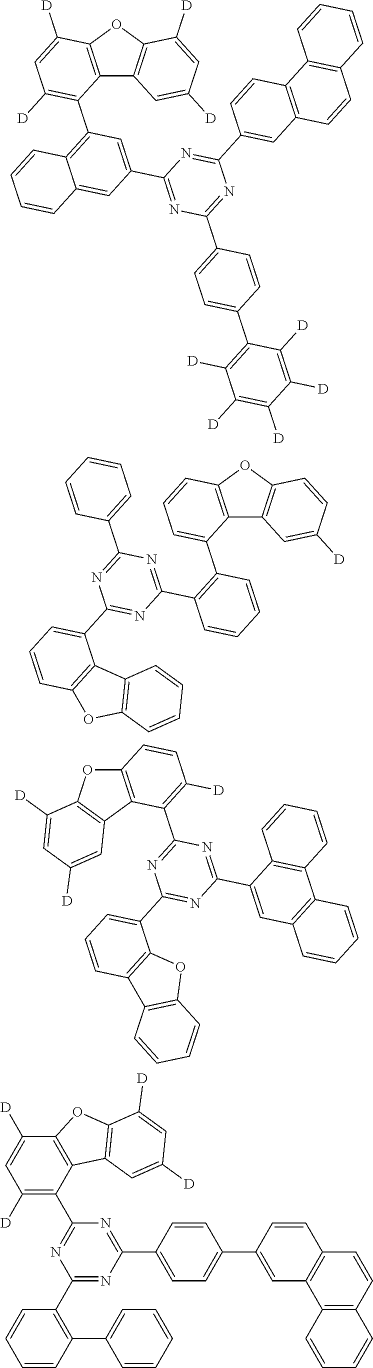

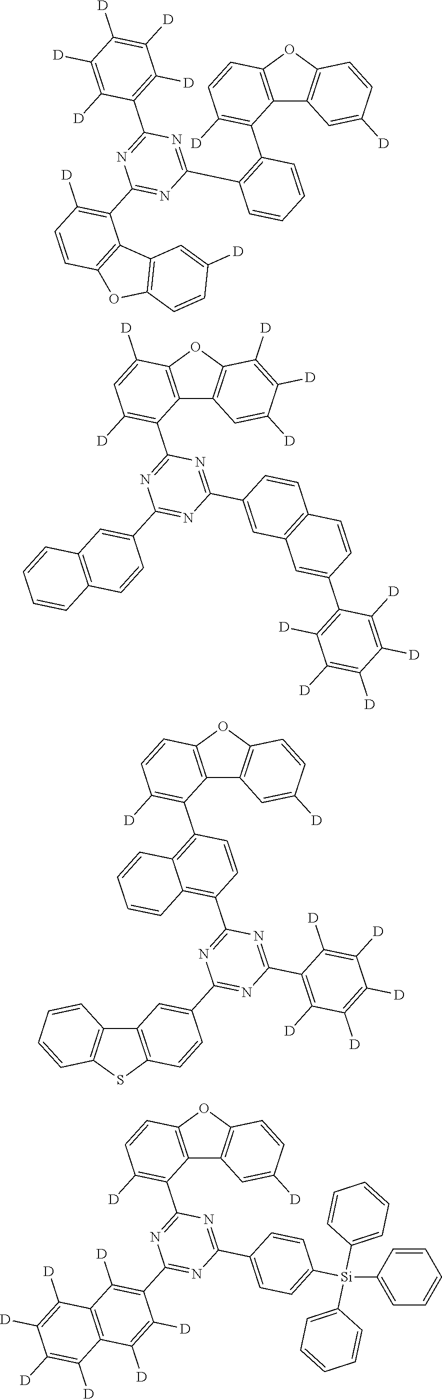

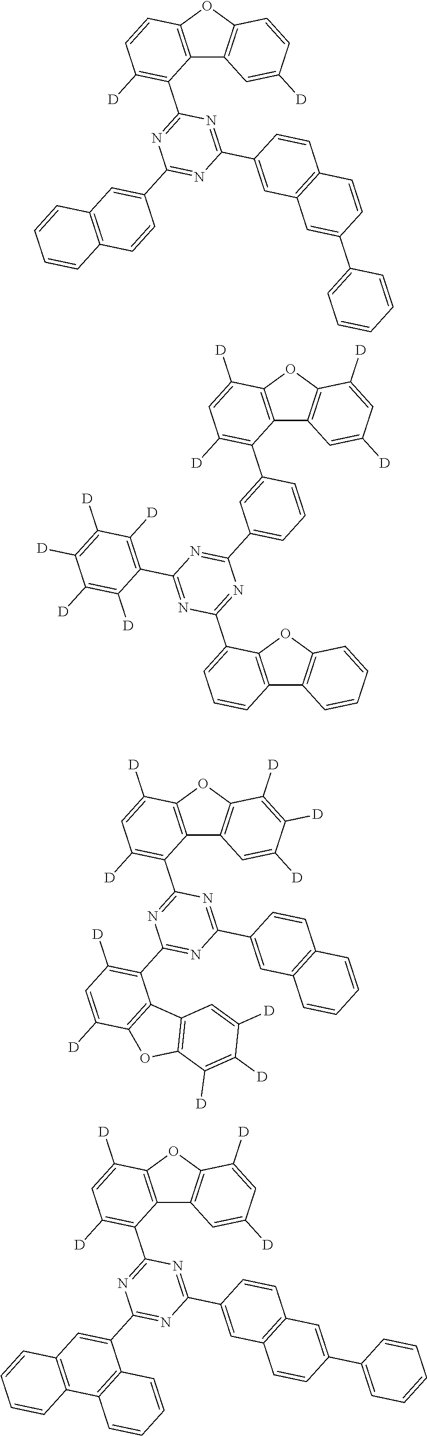

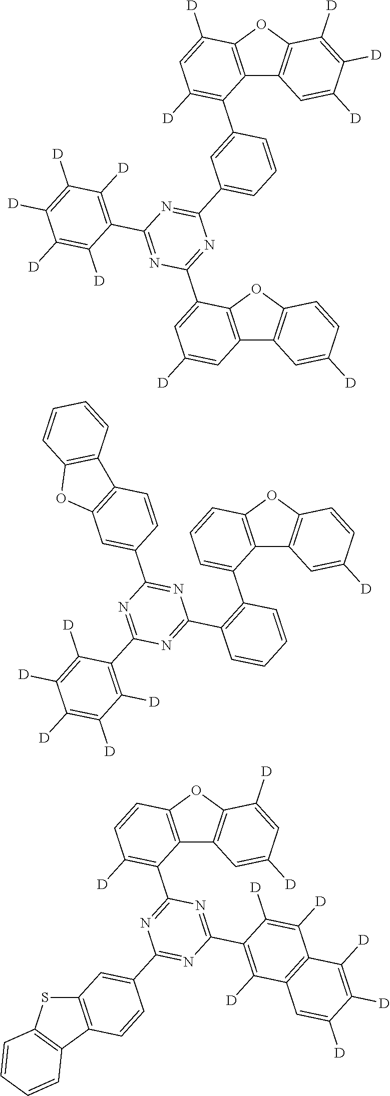 [SMILES: [2H]c1ccc2oc3c(-c4nc(-c5cccc(-c6c([2H])cc([2H])c7oc8c([2H])c([2H])c([2H])cc8c67)c5)nc(-c5c([2H])c([2H])c([2H])c([2H])c5[2H])n4)cc([2H])cc3c2c1 ; [2H]c1ccc2oc3cccc(-c4ccccc4-c4nc(-c5ccc6c(c5)oc5ccccc56)nc(-c5c([2H])c([2H])c([2H])c([2H])c5[2H])n4)c3c2c1 ; [2H]c1cc([2H])c2oc3ccc([2H])c(-c4nc(-c5ccc6c(c5)sc5ccccc56)nc(-c5c([2H])c([2H])c6c([2H])c([2H])c([2H])cc6c5[2H])n4)c3c2c1]

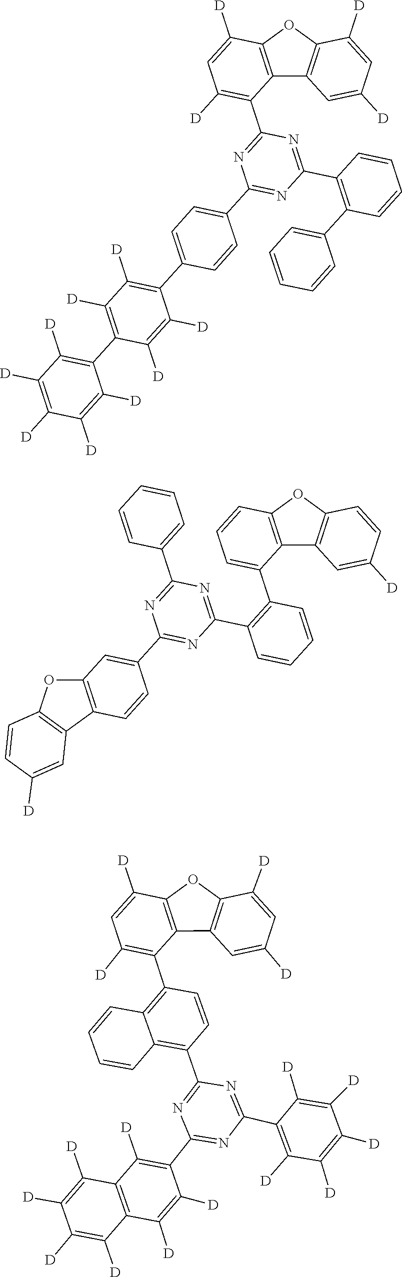 [SMILES: [2H]c1cc([2H])c2oc3c([2H])cc([2H])c(-c4nc(-c5ccc(-c6c([2H])c([2H])c(-c7c([2H])c([2H])c([2H])c([2H])c7[2H])c([2H])c6[2H])cc5)nc(-c5ccccc5-c5ccccc5)n4)c3c2c1 ; [2H]c1ccc2oc3cc(-c4nc(-c5ccccc5)nc(-c5ccccc5-c5cccc6oc7ccc([2H])cc7c56)n4)ccc3c2c1 ; [2H]c1cc([2H])c2oc3c([2H])cc([2H])c(-c4ccc(-c5nc(-c6c([2H])c([2H])c([2H])c([2H])c6[2H])nc(-c6c([2H])c([2H])c7c([2H])c([2H])c([2H])c([2H])c7c6[2H])n5)c5ccccc45)c3c2c1]

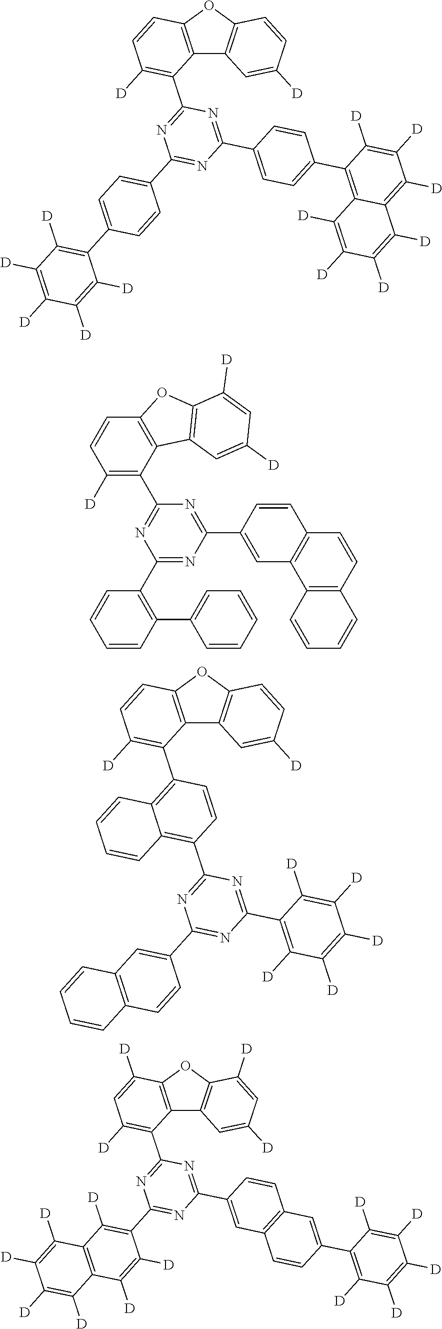

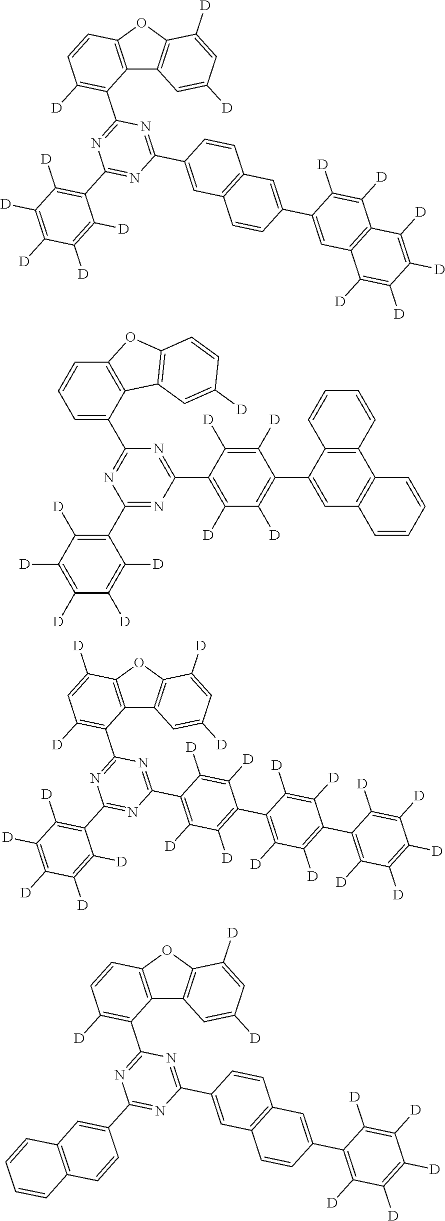

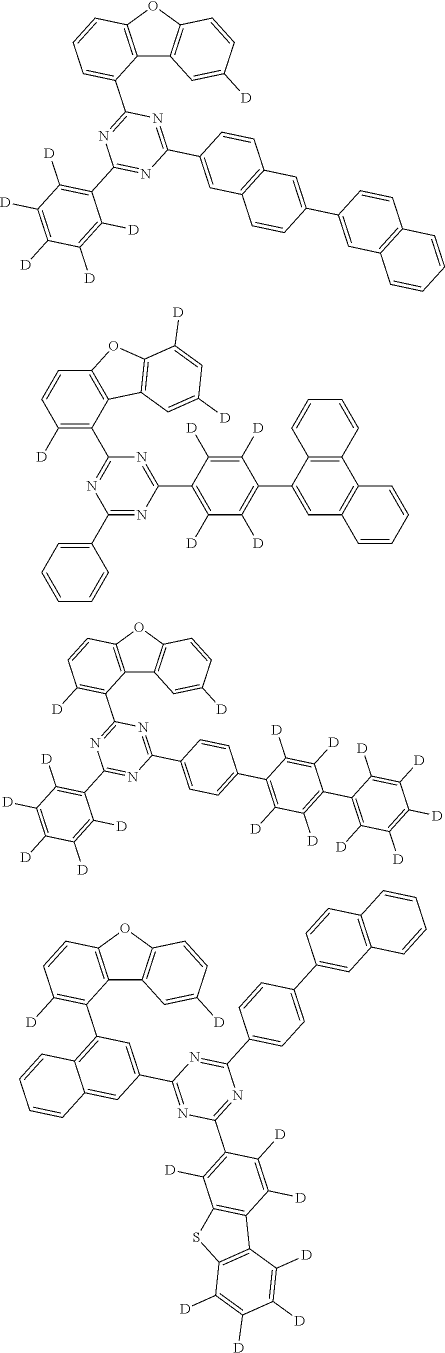

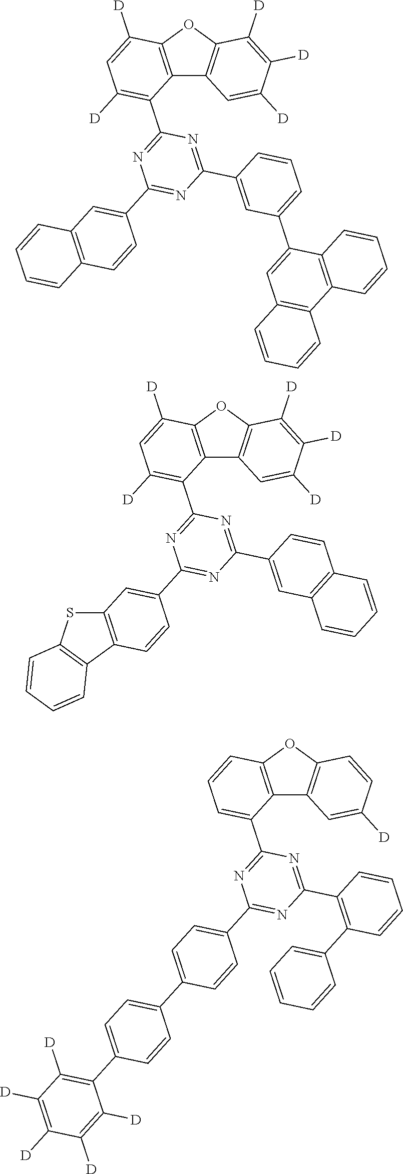

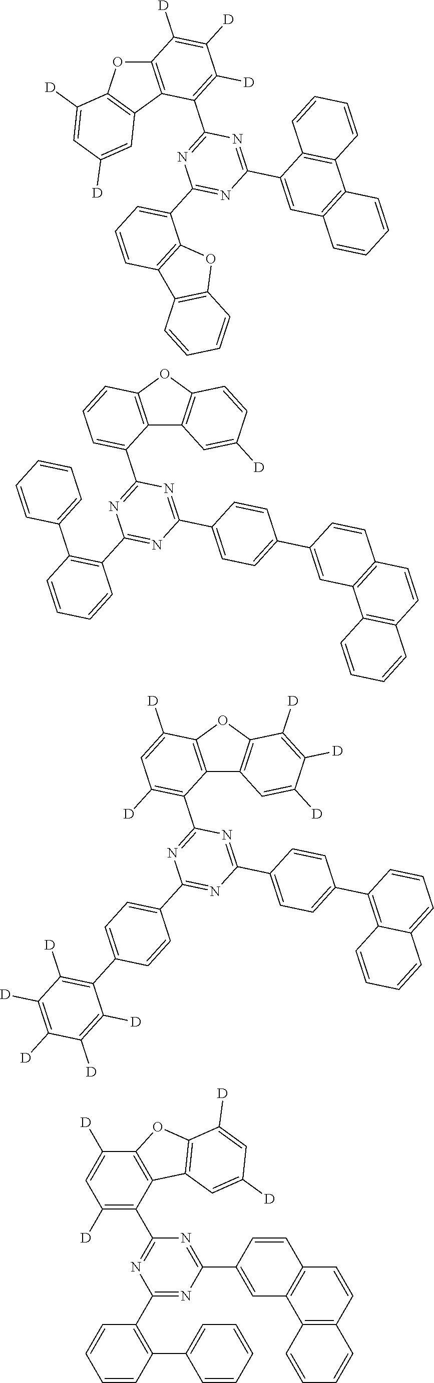

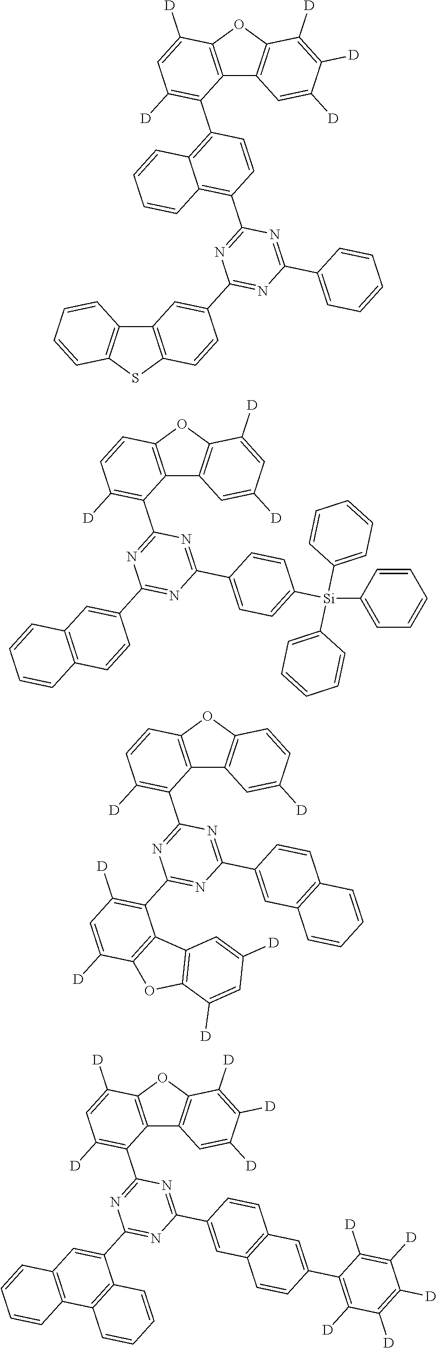

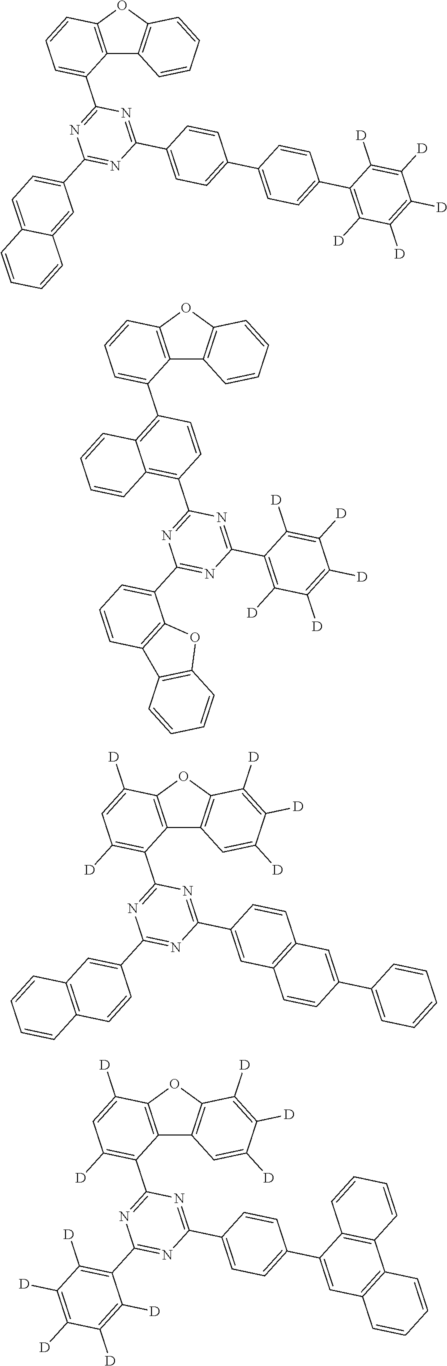

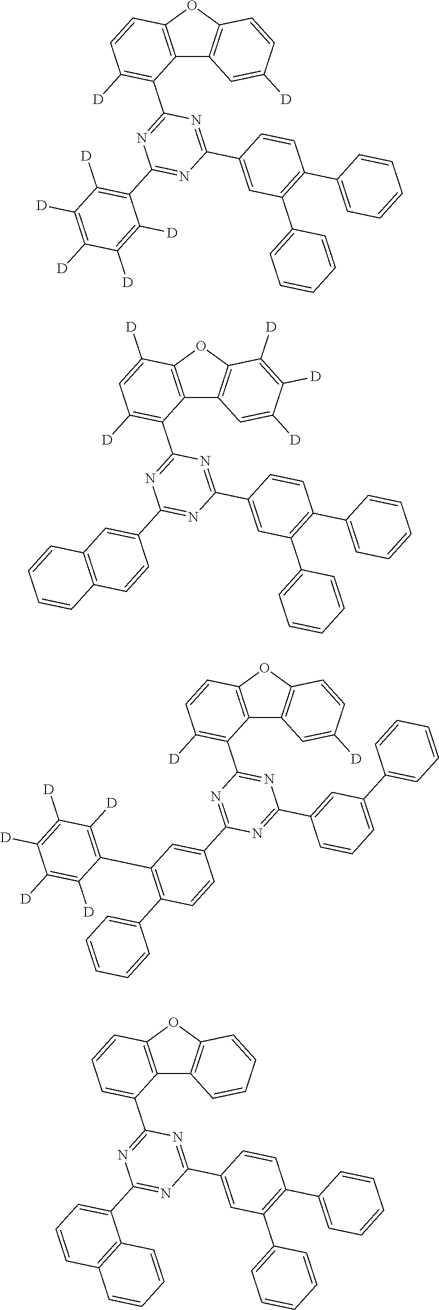

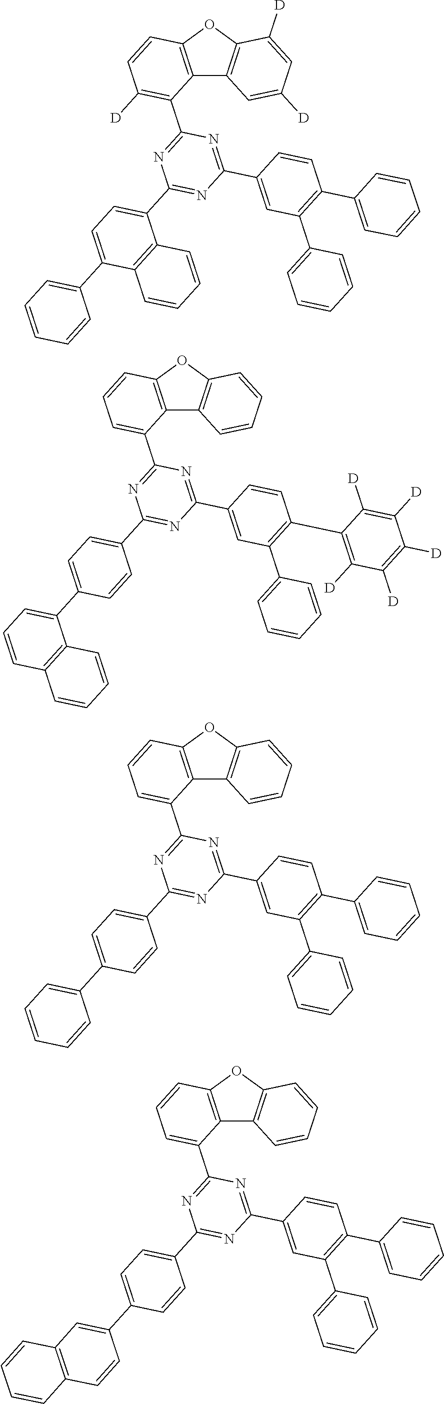 [SMILES: [2H]c1cc([2H])c2oc3ccc([2H])c(-c4nc(-c5ccc(-c6ccccc6)c(-c6ccccc6)c5)nc(-c5ccc(-c6ccccc6)c6ccccc56)n4)c3c2c1 ; [2H]c1c([2H])c([2H])c(-c2ccc(-c3nc(-c4ccc(-c5cccc6ccccc56)cc4)nc(-c4cccc5oc6ccccc6c45)n3)cc2-c2ccccc2)c([2H])c1[2H] ; c1ccc(-c2ccc(-c3nc(-c4ccc(-c5ccccc5)c(-c5ccccc5)c4)nc(-c4cccc5oc6ccccc6c45)n3)cc2)cc1 ; c1ccc(-c2ccc(-c3nc(-c4ccc(-c5ccc6ccccc6c5)cc4)nc(-c4cccc5oc6ccccc6c45)n3)cc2-c2ccccc2)cc1]

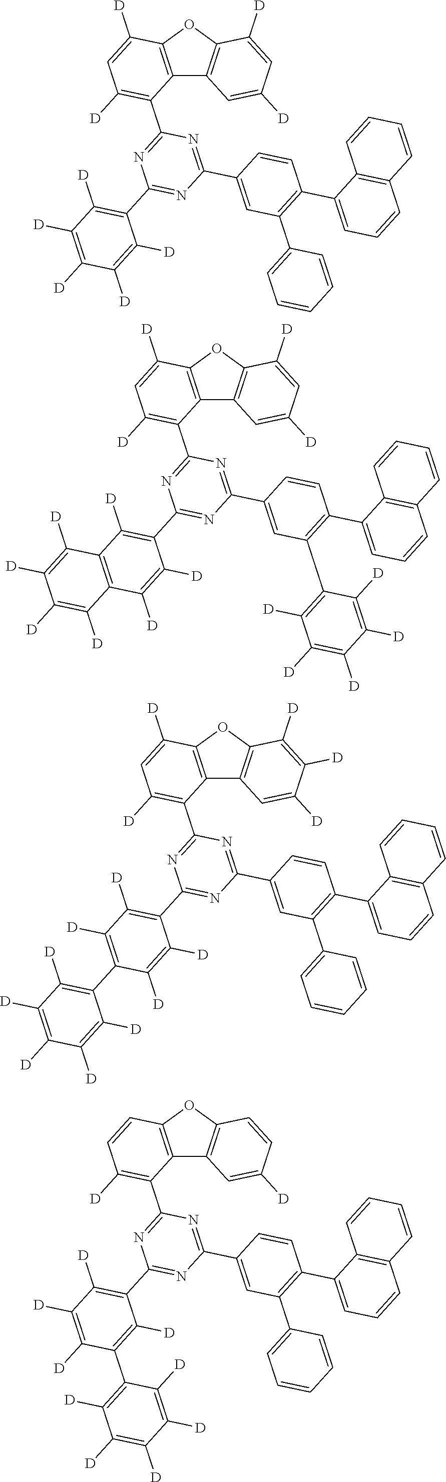

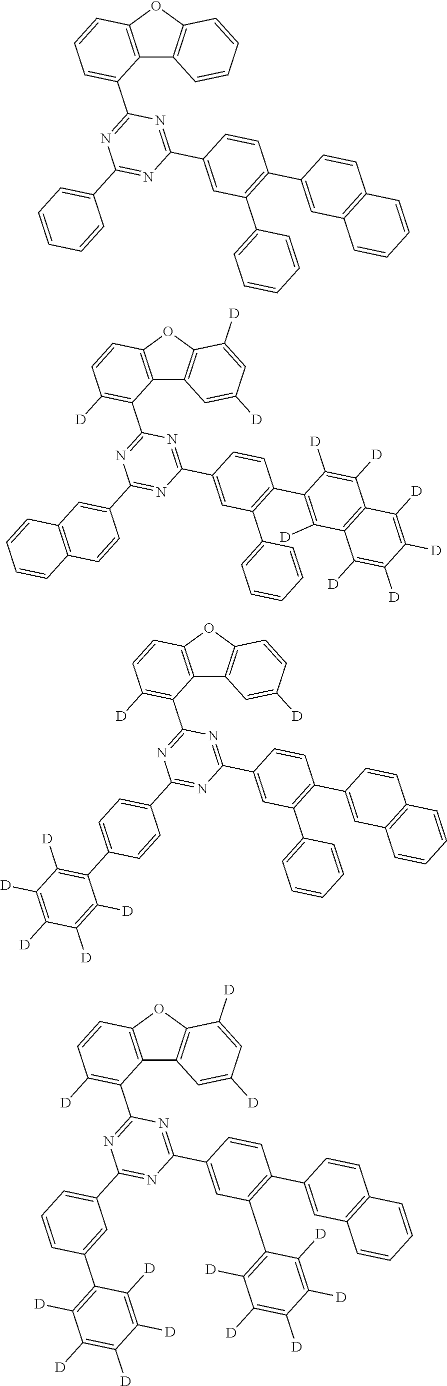

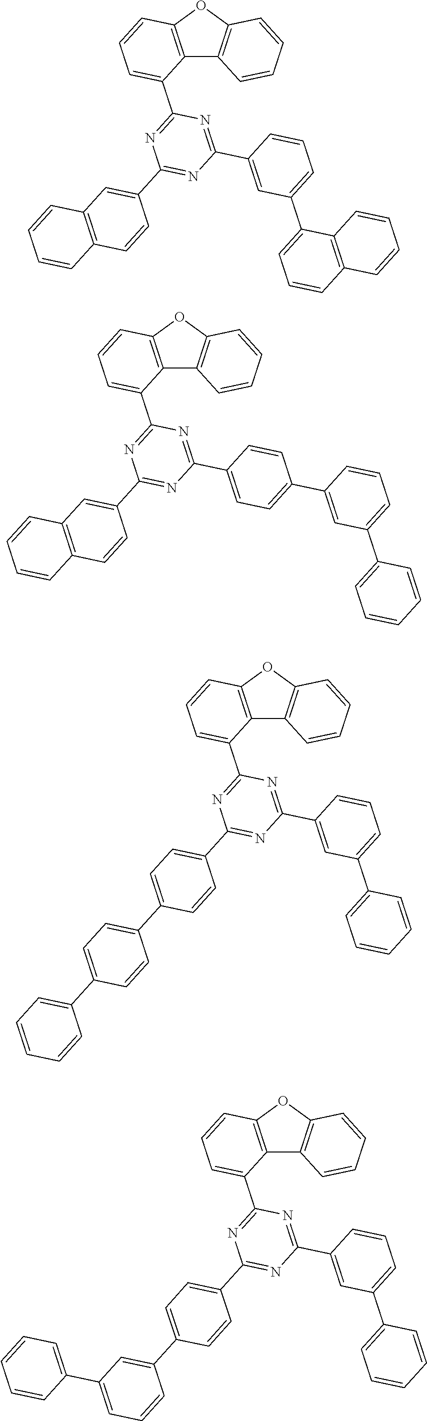 [SMILES: c1cc(-c2nc(-c3ccc4ccccc4c3)nc(-c3cccc4oc5ccccc5c34)n2)cc(-c2cccc3ccccc23)c1 ; c1ccc(-c2cccc(-c3ccc(-c4nc(-c5ccc6ccccc6c5)nc(-c5cccc6oc7ccccc7c56)n4)cc3)c2)cc1 ; c1ccc(-c2ccc(-c3ccc(-c4nc(-c5cccc(-c6ccccc6)c5)nc(-c5cccc6oc7ccccc7c56)n4)cc3)cc2)cc1 ; c1ccc(-c2cccc(-c3ccc(-c4nc(-c5cccc(-c6ccccc6)c5)nc(-c5cccc6oc7ccccc7c56)n4)cc3)c2)cc1]

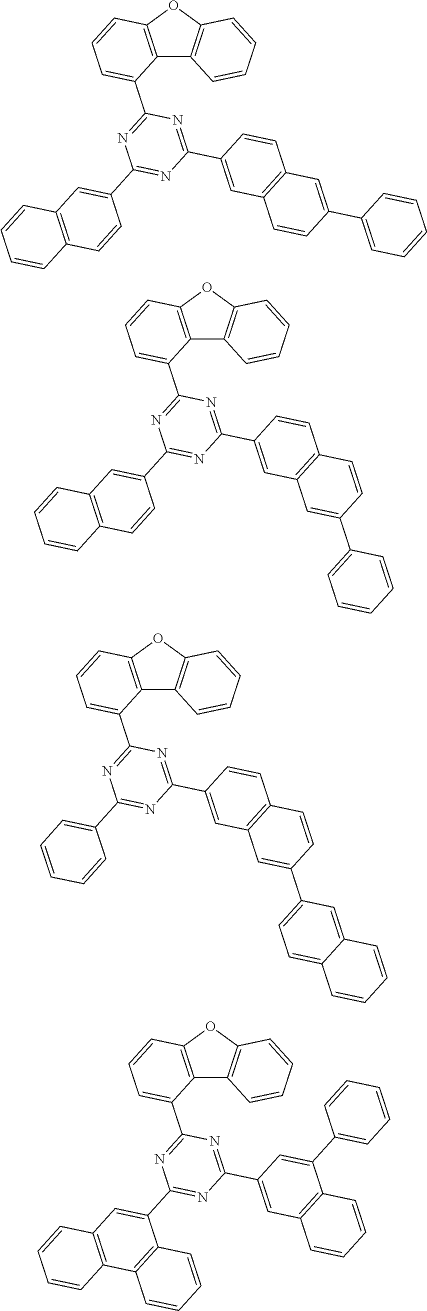 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc3ccc(-c4nc(-c5ccc6ccccc6c5)nc(-c5cccc6oc7ccccc7c56)n4)cc3c2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2cc(-c3nc(-c4cc5ccccc5c5ccccc45)nc(-c4cccc5oc6ccccc6c45)n3)cc3ccccc23)cc1]

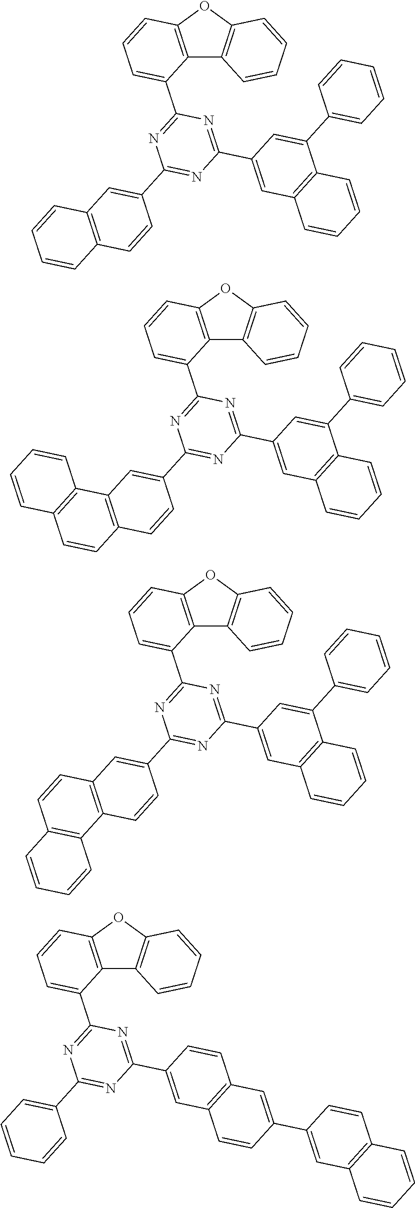

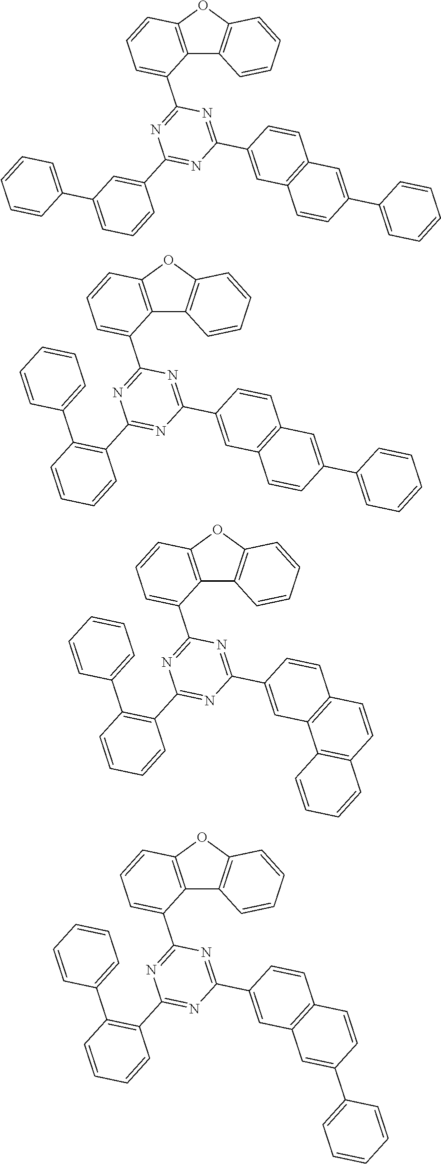

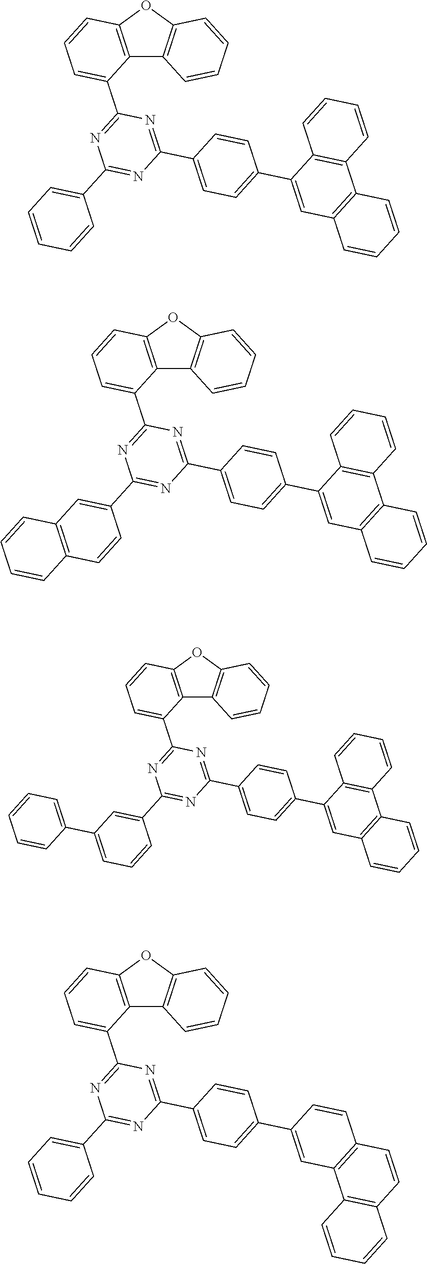 [SMILES: c1ccc(-c2nc(-c3ccc(-c4cc5ccccc5c5ccccc45)cc3)nc(-c3cccc4oc5ccccc5c34)n2)cc1 ; c1ccc2cc(-c3nc(-c4ccc(-c5cc6ccccc6c6ccccc56)cc4)nc(-c4cccc5oc6ccccc6c45)n3)ccc2c1 ; c1ccc(-c2cccc(-c3nc(-c4ccc(-c5cc6ccccc6c6ccccc56)cc4)nc(-c4cccc5oc6ccccc6c45)n3)c2)cc1 ; c1ccc(-c2nc(-c3ccc(-c4ccc5ccc6ccccc6c5c4)cc3)nc(-c3cccc4oc5ccccc5c34)n2)cc1]

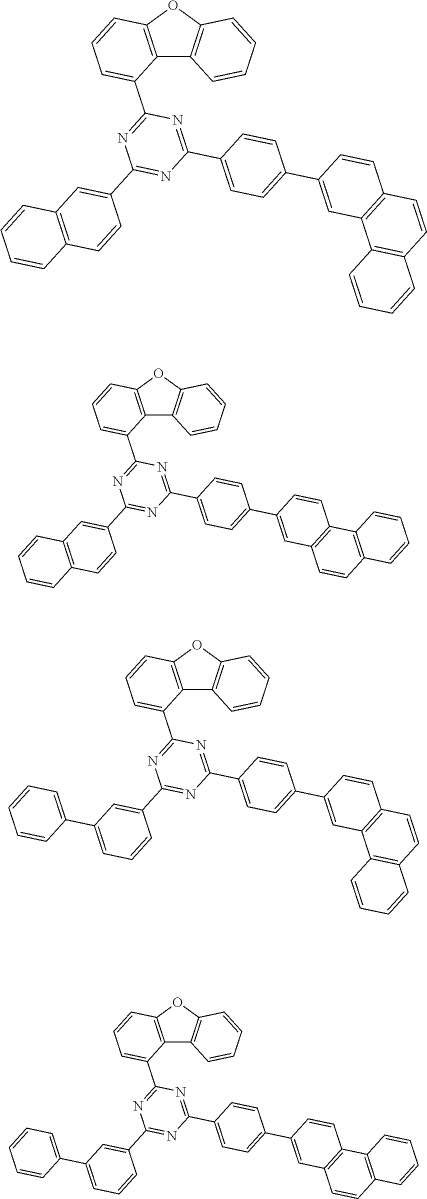 [SMILES: c1ccc2cc(-c3nc(-c4ccc(-c5ccc6ccc7ccccc7c6c5)cc4)nc(-c4cccc5oc6ccccc6c45)n3)ccc2c1 ; c1ccc2cc(-c3nc(-c4ccc(-c5ccc6c(ccc7ccccc76)c5)cc4)nc(-c4cccc5oc6ccccc6c45)n3)ccc2c1 ; c1ccc(-c2cccc(-c3nc(-c4ccc(-c5ccc6ccc7ccccc7c6c5)cc4)nc(-c4cccc5oc6ccccc6c45)n3)c2)cc1 ; c1ccc(-c2cccc(-c3nc(-c4ccc(-c5ccc6c(ccc7ccccc76)c5)cc4)nc(-c4cccc5oc6ccccc6c45)n3)c2)cc1]

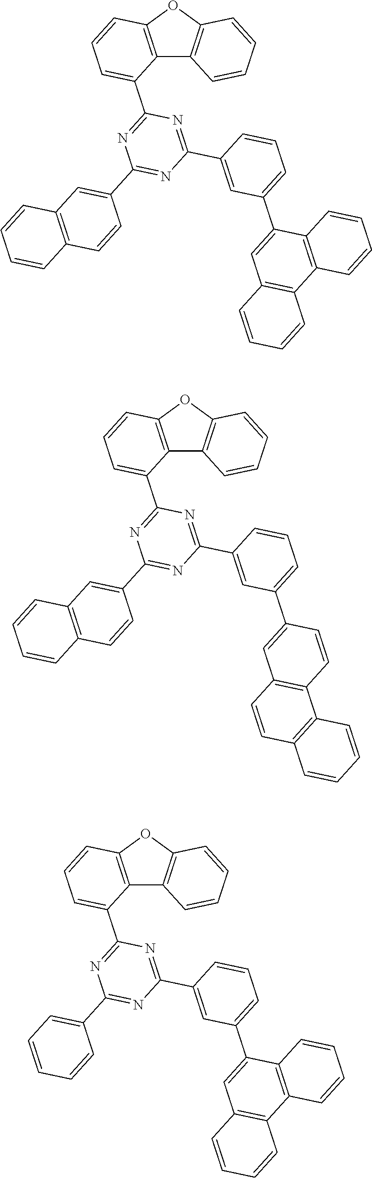 [SMILES: c1cc(-c2nc(-c3ccc4ccccc4c3)nc(-c3cccc4oc5ccccc5c34)n2)cc(-c2cc3ccccc3c3ccccc23)c1 ; c1cc(-c2ccc3c(ccc4ccccc43)c2)cc(-c2nc(-c3ccc4ccccc4c3)nc(-c3cccc4oc5ccccc5c34)n2)c1 ; c1ccc(-c2nc(-c3cccc(-c4cc5ccccc5c5ccccc45)c3)nc(-c3cccc4oc5ccccc5c34)n2)cc1]

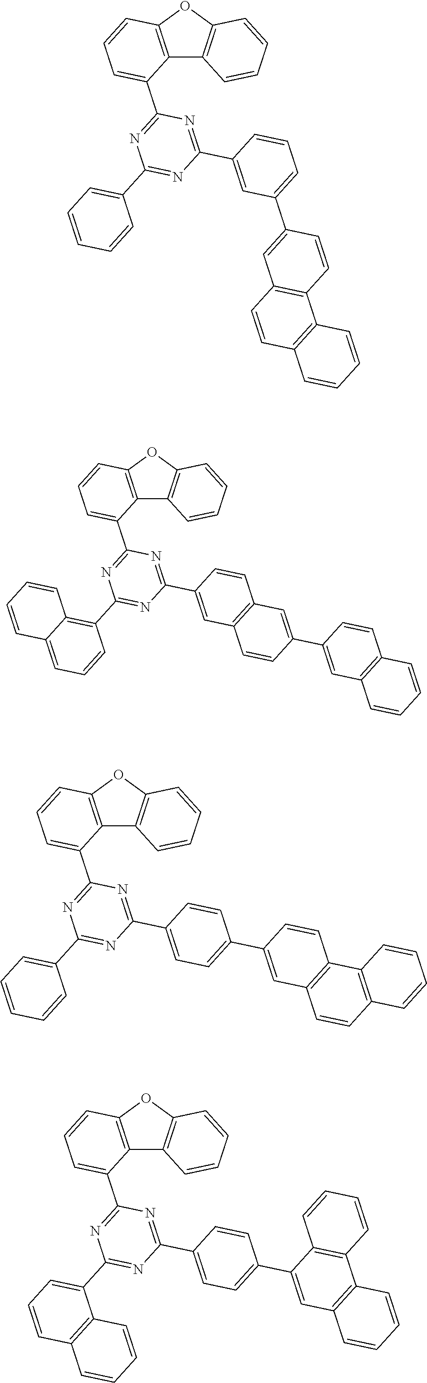 [SMILES: c1ccc(-c2nc(-c3cccc(-c4ccc5c(ccc6ccccc65)c4)c3)nc(-c3cccc4oc5ccccc5c34)n2)cc1 ; c1ccc2cc(-c3ccc4cc(-c5nc(-c6cccc7ccccc67)nc(-c6cccc7oc8ccccc8c67)n5)ccc4c3)ccc2c1 ; c1ccc(-c2nc(-c3ccc(-c4ccc5c(ccc6ccccc65)c4)cc3)nc(-c3cccc4oc5ccccc5c34)n2)cc1 ; c1ccc2c(-c3nc(-c4ccc(-c5cc6ccccc6c6ccccc56)cc4)nc(-c4cccc5oc6ccccc6c45)n3)cccc2c1]

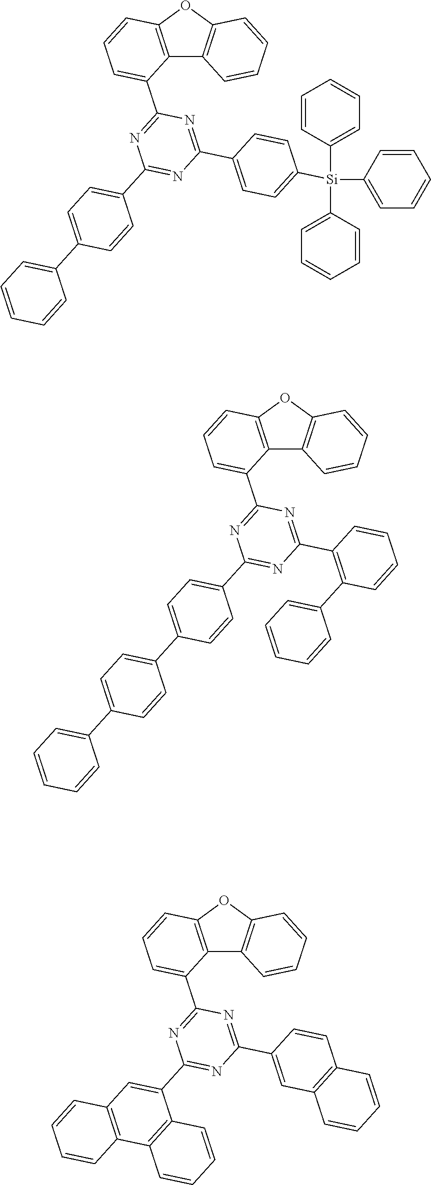 [SMILES: c1ccc(-c2ccc(-c3nc(-c4ccc([Si](c5ccccc5)(c5ccccc5)c5ccccc5)cc4)nc(-c4cccc5oc6ccccc6c45)n3)cc2)cc1 ; c1ccc(-c2ccc(-c3ccc(-c4nc(-c5ccccc5-c5ccccc5)nc(-c5cccc6oc7ccccc7c56)n4)cc3)cc2)cc1 ; c1ccc2cc(-c3nc(-c4cc5ccccc5c5ccccc45)nc(-c4cccc5oc6ccccc6c45)n3)ccc2c1]

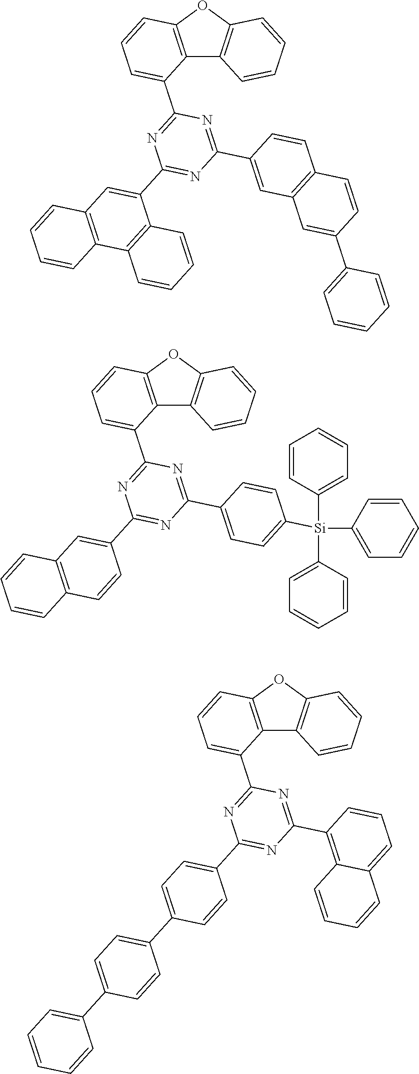 [SMILES: c1ccc(-c2ccc3ccc(-c4nc(-c5cc6ccccc6c6ccccc56)nc(-c5cccc6oc7ccccc7c56)n4)cc3c2)cc1 ; c1ccc([Si](c2ccccc2)(c2ccccc2)c2ccc(-c3nc(-c4ccc5ccccc5c4)nc(-c4cccc5oc6ccccc6c45)n3)cc2)cc1 ; c1ccc(-c2ccc(-c3ccc(-c4nc(-c5cccc6ccccc56)nc(-c5cccc6oc7ccccc7c56)n4)cc3)cc2)cc1]

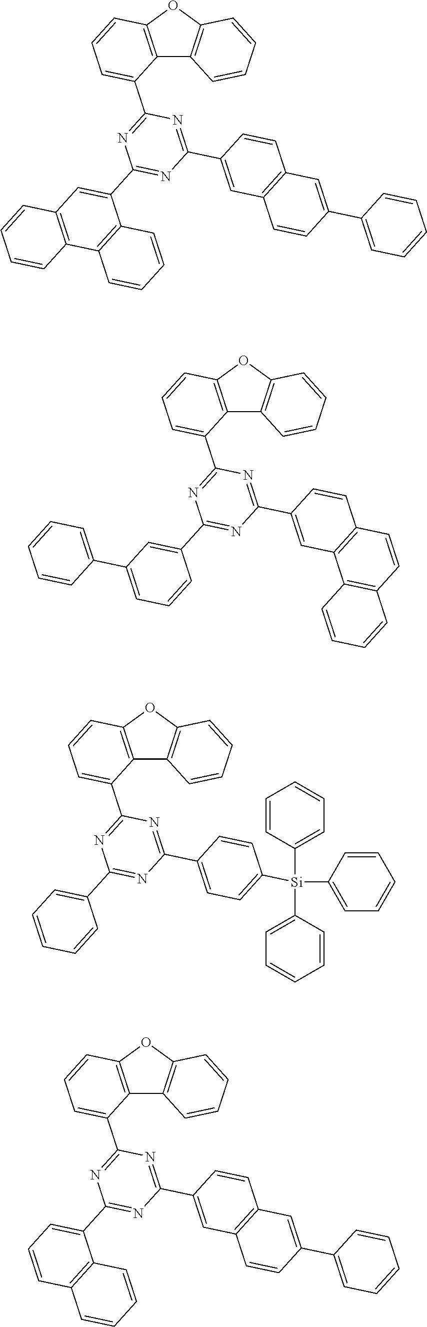 [SMILES: c1ccc(-c2ccc3cc(-c4nc(-c5cc6ccccc6c6ccccc56)nc(-c5cccc6oc7ccccc7c56)n4)ccc3c2)cc1 ; c1ccc(-c2cccc(-c3nc(-c4ccc5ccc6ccccc6c5c4)nc(-c4cccc5oc6ccccc6c45)n3)c2)cc1 ; c1ccc(-c2nc(-c3ccc([Si](c4ccccc4)(c4ccccc4)c4ccccc4)cc3)nc(-c3cccc4oc5ccccc5c34)n2)cc1 ; c1ccc(-c2ccc3cc(-c4nc(-c5cccc6ccccc56)nc(-c5cccc6oc7ccccc7c56)n4)ccc3c2)cc1]

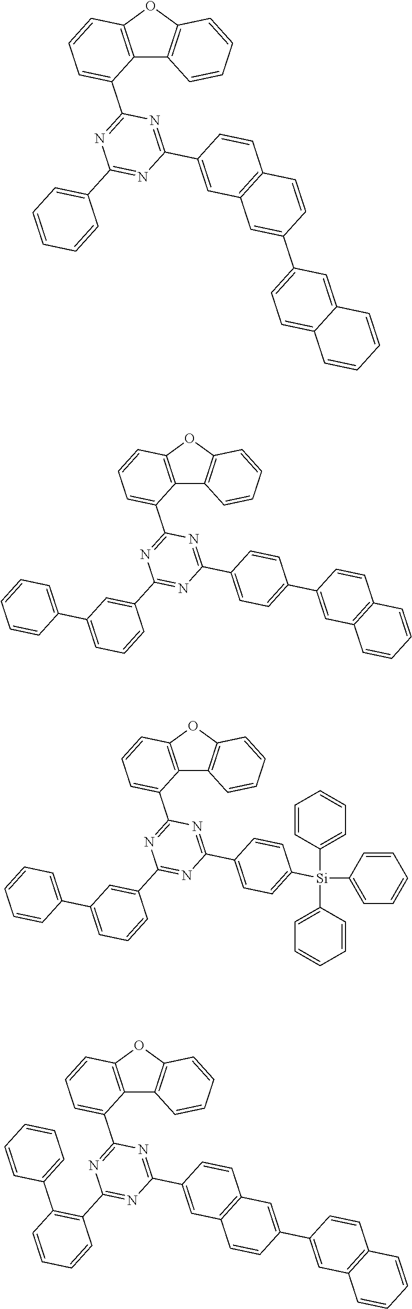 [SMILES: c1ccc(-c2nc(-c3ccc4ccc(-c5ccc6ccccc6c5)cc4c3)nc(-c3cccc4oc5ccccc5c34)n2)cc1 ; c1ccc(-c2cccc(-c3nc(-c4ccc(-c5ccc6ccccc6c5)cc4)nc(-c4cccc5oc6ccccc6c45)n3)c2)cc1 ; c1ccc(-c2cccc(-c3nc(-c4ccc([Si](c5ccccc5)(c5ccccc5)c5ccccc5)cc4)nc(-c4cccc5oc6ccccc6c45)n3)c2)cc1 ; c1ccc(-c2ccccc2-c2nc(-c3ccc4cc(-c5ccc6ccccc6c5)ccc4c3)nc(-c3cccc4oc5ccccc5c34)n2)cc1]

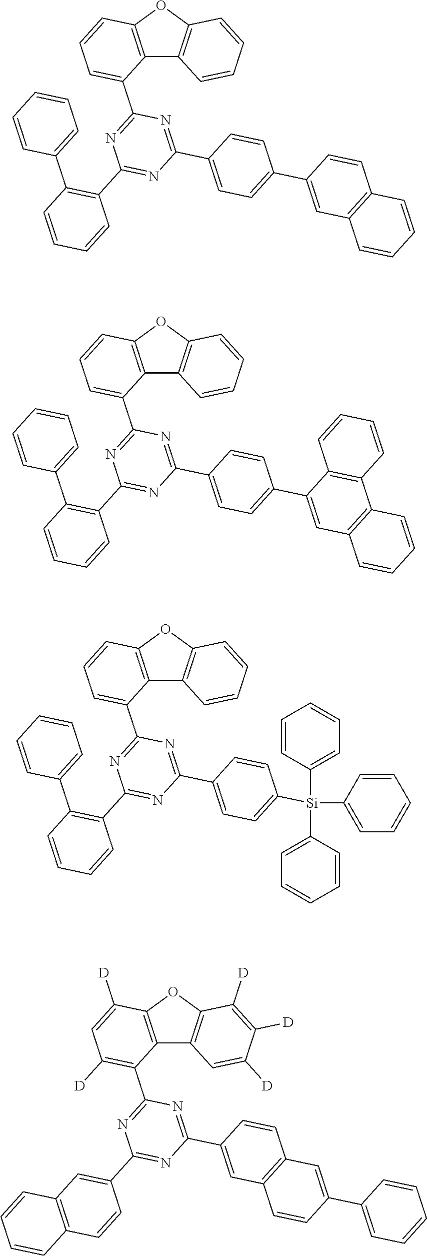 [SMILES: c1ccc(-c2ccccc2-c2nc(-c3ccc(-c4ccc5ccccc5c4)cc3)nc(-c3cccc4oc5ccccc5c34)n2)cc1 ; c1ccc(-c2ccccc2-c2nc(-c3ccc(-c4cc5ccccc5c5ccccc45)cc3)nc(-c3cccc4oc5ccccc5c34)n2)cc1 ; c1ccc(-c2ccccc2-c2nc(-c3ccc([Si](c4ccccc4)(c4ccccc4)c4ccccc4)cc3)nc(-c3cccc4oc5ccccc5c34)n2)cc1 ; [2H]c1cc2c(oc3c([2H])cc([2H])c(-c4nc(-c5ccc6ccccc6c5)nc(-c5ccc6cc(-c7ccccc7)ccc6c5)n4)c32)c([2H])c1[2H]]

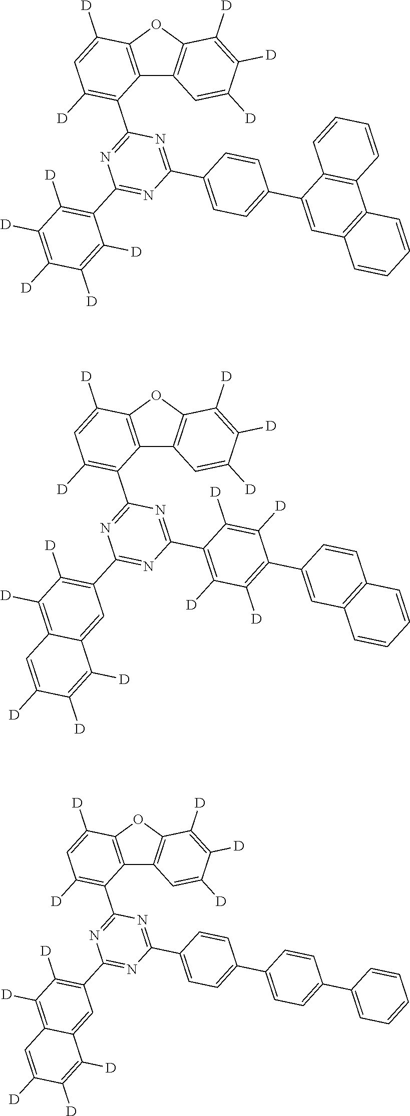 [SMILES: [2H]c1cc2c(oc3c([2H])cc([2H])c(-c4nc(-c5ccc(-c6cc7ccccc7c7ccccc67)cc5)nc(-c5c([2H])c([2H])c([2H])c([2H])c5[2H])n4)c32)c([2H])c1[2H] ; [2H]c1cc2c([2H])c([2H])c(-c3nc(-c4c([2H])c([2H])c(-c5ccc6ccccc6c5)c([2H])c4[2H])nc(-c4c([2H])cc([2H])c5oc6c([2H])c([2H])c([2H])cc6c45)n3)cc2c([2H])c1[2H] ; [2H]c1cc2c([2H])c([2H])c(-c3nc(-c4ccc(-c5ccc(-c6ccccc6)cc5)cc4)nc(-c4c([2H])cc([2H])c5oc6c([2H])c([2H])c([2H])cc6c45)n3)cc2c([2H])c1[2H]]

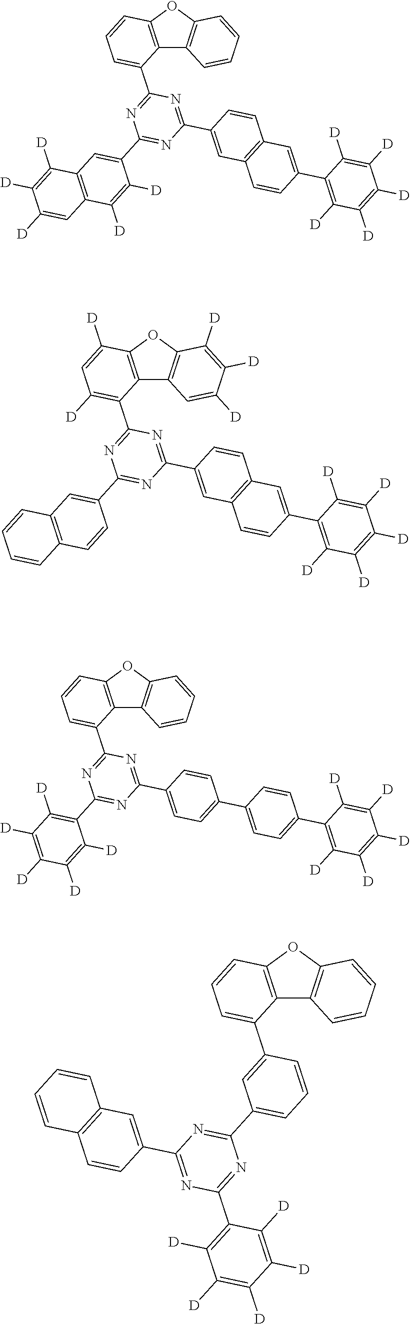 [SMILES: [2H]c1cc2c([2H])c([2H])c(-c3nc(-c4ccc5cc(-c6c([2H])c([2H])c([2H])c([2H])c6[2H])ccc5c4)nc(-c4cccc5oc6ccccc6c45)n3)cc2c([2H])c1[2H] ; [2H]c1cc2c(oc3c([2H])cc([2H])c(-c4nc(-c5ccc6ccccc6c5)nc(-c5ccc6cc(-c7c([2H])c([2H])c([2H])c([2H])c7[2H])ccc6c5)n4)c32)c([2H])c1[2H] ; [2H]c1c([2H])c([2H])c(-c2ccc(-c3ccc(-c4nc(-c5c([2H])c([2H])c([2H])c([2H])c5[2H])nc(-c5cccc6oc7ccccc7c56)n4)cc3)cc2)c([2H])c1[2H] ; [2H]c1c([2H])c([2H])c(-c2nc(-c3cccc(-c4cccc5oc6ccccc6c45)c3)nc(-c3ccc4ccccc4c3)n2)c([2H])c1[2H]]

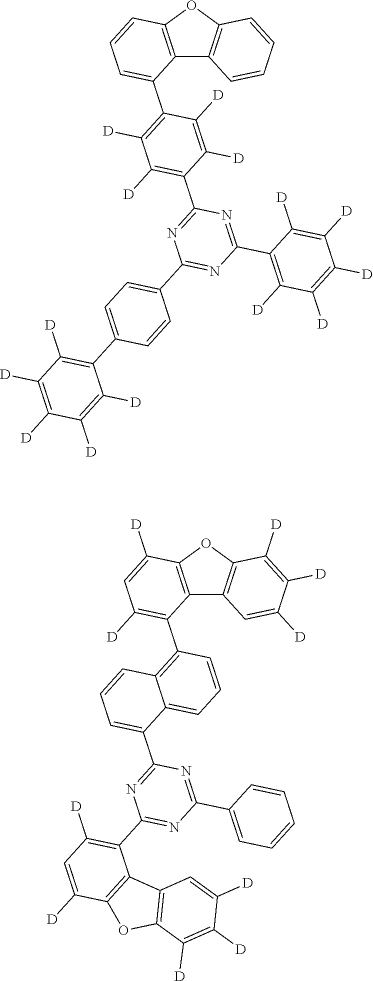 [SMILES: [2H]c1c([2H])c([2H])c(-c2ccc(-c3nc(-c4c([2H])c([2H])c([2H])c([2H])c4[2H])nc(-c4c([2H])c([2H])c(-c5cccc6oc7ccccc7c56)c([2H])c4[2H])n3)cc2)c([2H])c1[2H] ; [2H]c1cc2c(oc3c([2H])cc([2H])c(-c4nc(-c5ccccc5)nc(-c5cccc6c(-c7c([2H])cc([2H])c8oc9c([2H])c([2H])c([2H])cc9c78)cccc56)n4)c32)c([2H])c1[2H]]

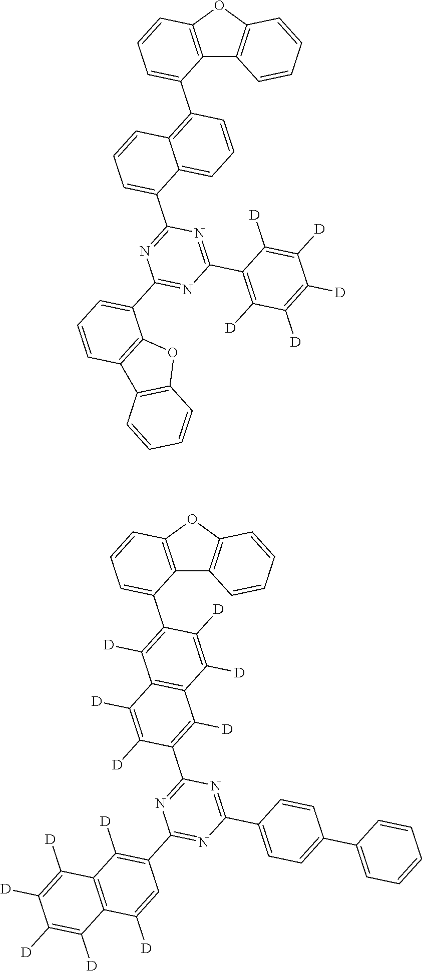 [SMILES: [2H]c1c([2H])c([2H])c(-c2nc(-c3cccc4c(-c5cccc6oc7ccccc7c56)cccc34)nc(-c3cccc4c3oc3ccccc34)n2)c([2H])c1[2H] ; [2H]c1c([2H])c([2H])c2c([2H])c(-c3nc(-c4ccc(-c5ccccc5)cc4)nc(-c4c([2H])c([2H])c5c([2H])c(-c6cccc7oc8ccccc8c67)c([2H])c([2H])c5c4[2H])n3)cc([2H])c2c1[2H]]

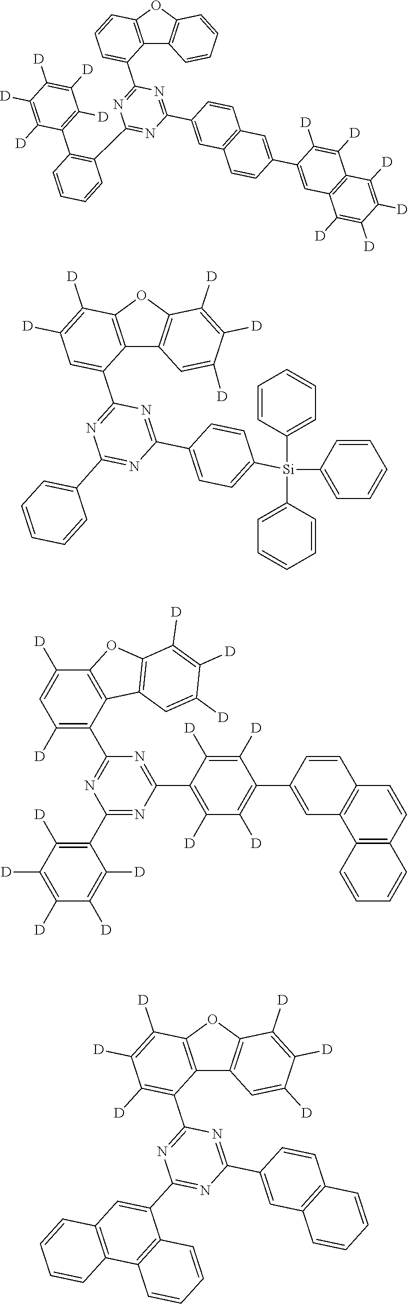

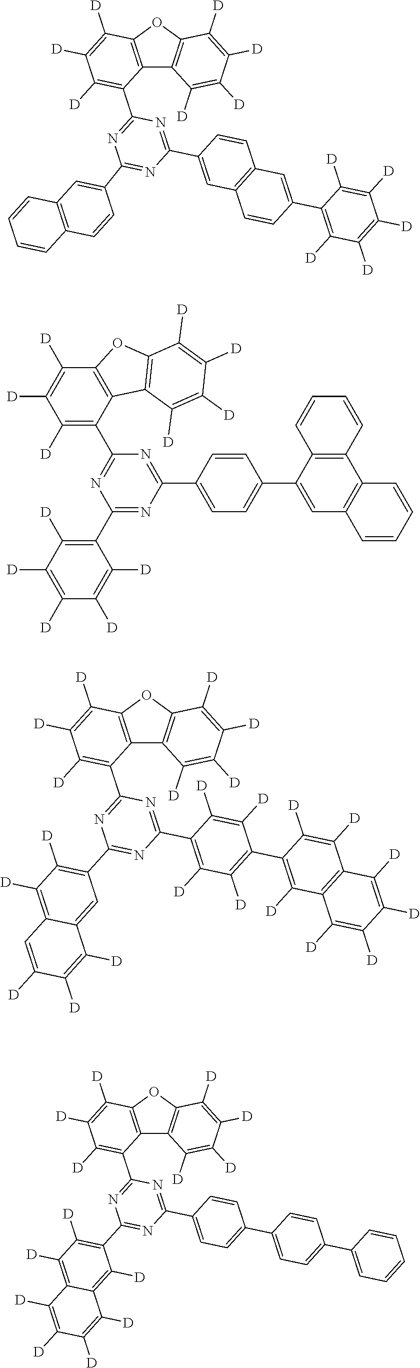

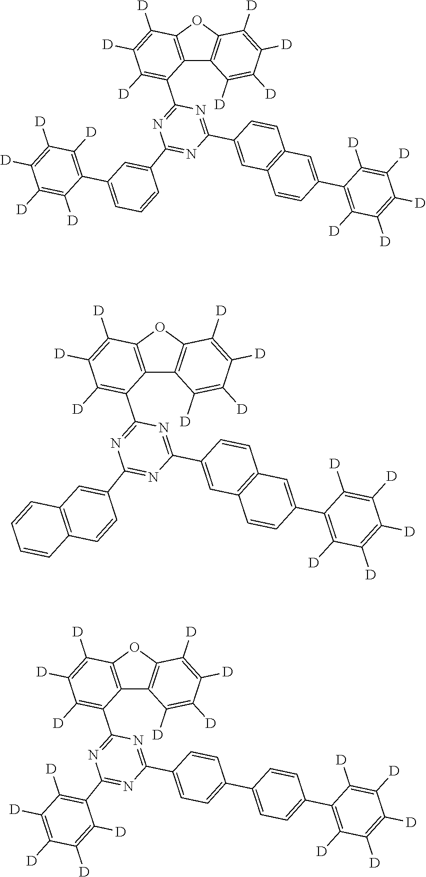 [SMILES: [2H]c1c([2H])c([2H])c(-c2cccc(-c3nc(-c4ccc5cc(-c6c([2H])c([2H])c([2H])c([2H])c6[2H])ccc5c4)nc(-c4c([2H])c([2H])c([2H])c5oc6c([2H])c([2H])c([2H])c([2H])c6c45)n3)c2)c([2H])c1[2H] ; [2H]c1c([2H])c([2H])c(-c2ccc3cc(-c4nc(-c5ccc6ccccc6c5)nc(-c5c([2H])c([2H])c([2H])c6oc7c([2H])c([2H])c([2H])c([2H])c7c56)n4)ccc3c2)c([2H])c1[2H] ; [2H]c1c([2H])c([2H])c(-c2ccc(-c3ccc(-c4nc(-c5c([2H])c([2H])c([2H])c([2H])c5[2H])nc(-c5c([2H])c([2H])c([2H])c6oc7c([2H])c([2H])c([2H])c([2H])c7c56)n4)cc3)cc2)c([2H])c1[2H]]

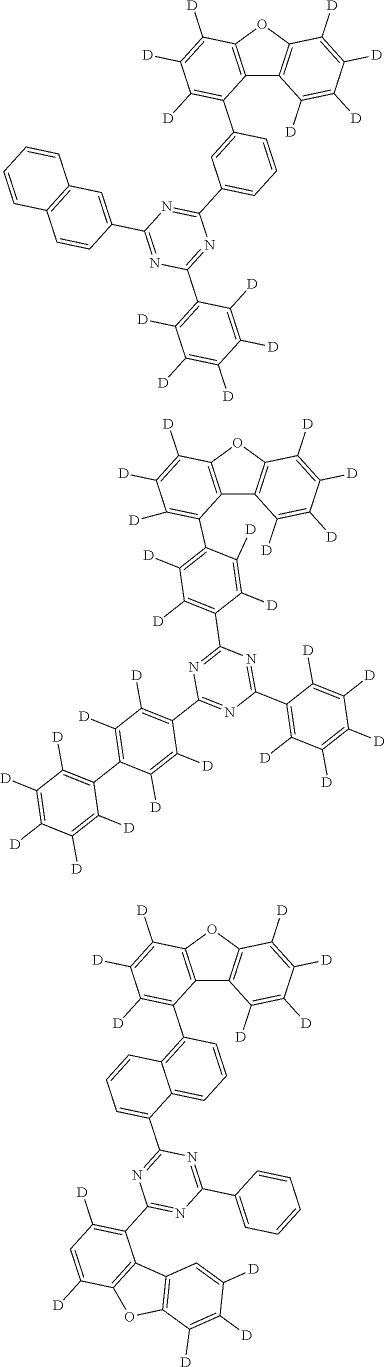

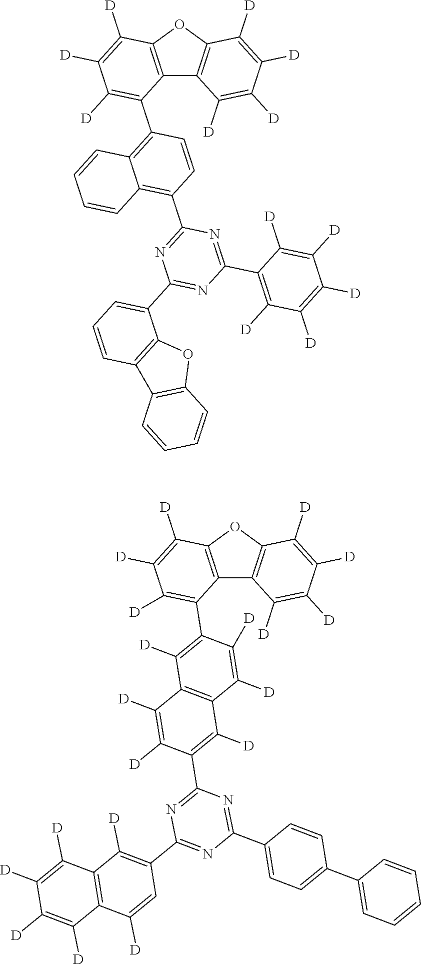 [SMILES: [2H]c1c([2H])c([2H])c(-c2nc(-c3ccc(-c4c([2H])c([2H])c([2H])c5oc6c([2H])c([2H])c([2H])c([2H])c6c45)c4ccccc34)nc(-c3cccc4c3oc3ccccc34)n2)c([2H])c1[2H] ; [2H]c1c([2H])c([2H])c2c(oc3c([2H])c([2H])c([2H])c(-c4c([2H])c([2H])c5c([2H])c(-c6nc(-c7ccc(-c8ccccc8)cc7)nc(-c7cc([2H])c8c([2H])c([2H])c([2H])c([2H])c8c7[2H])n6)c([2H])c([2H])c5c4[2H])c32)c1[2H]]

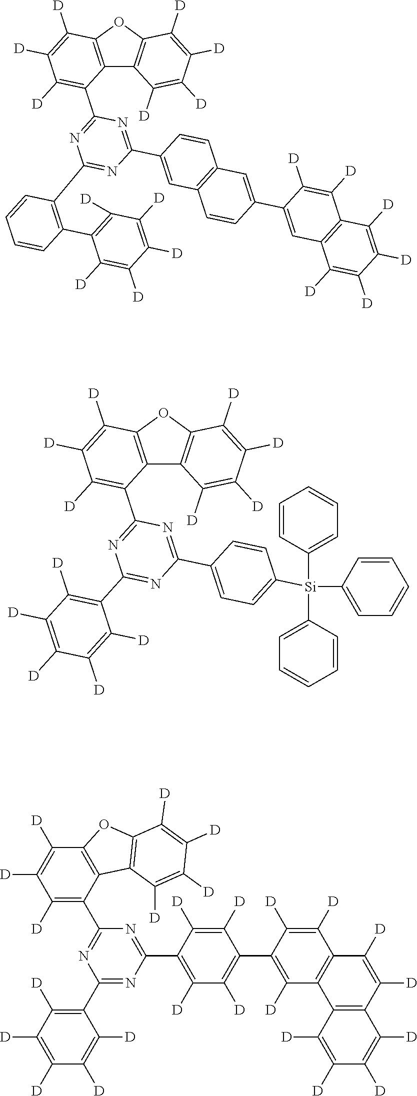

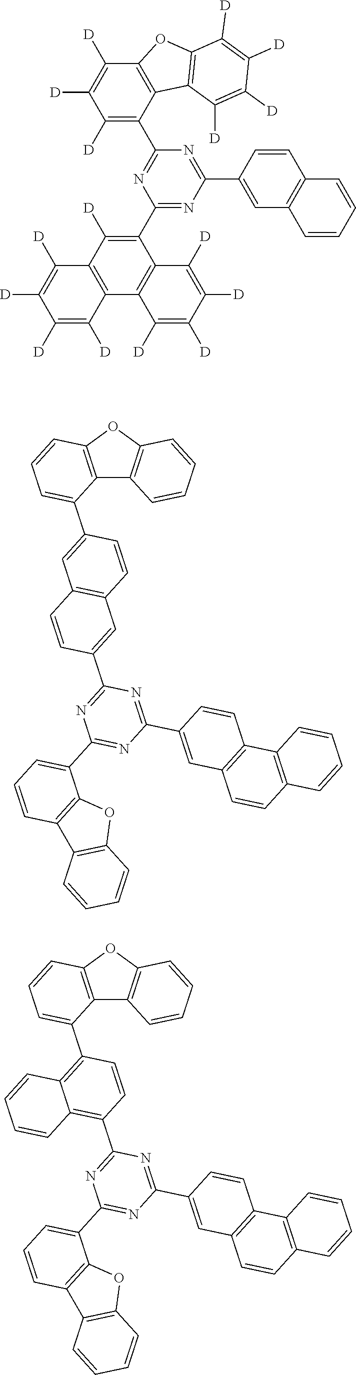 [SMILES: [2H]c1c([2H])c([2H])c2c(oc3c([2H])c([2H])c([2H])c(-c4nc(-c5ccc6ccccc6c5)nc(-c5c([2H])c6c([2H])c([2H])c([2H])c([2H])c6c6c([2H])c([2H])c([2H])c([2H])c56)n4)c32)c1[2H] ; c1ccc2c(c1)ccc1cc(-c3nc(-c4ccc5cc(-c6cccc7oc8ccccc8c67)ccc5c4)nc(-c4cccc5c4oc4ccccc45)n3)ccc12 ; c1ccc2c(c1)ccc1cc(-c3nc(-c4ccc(-c5cccc6oc7ccccc7c56)c5ccccc45)nc(-c4cccc5c4oc4ccccc45)n3)ccc12]

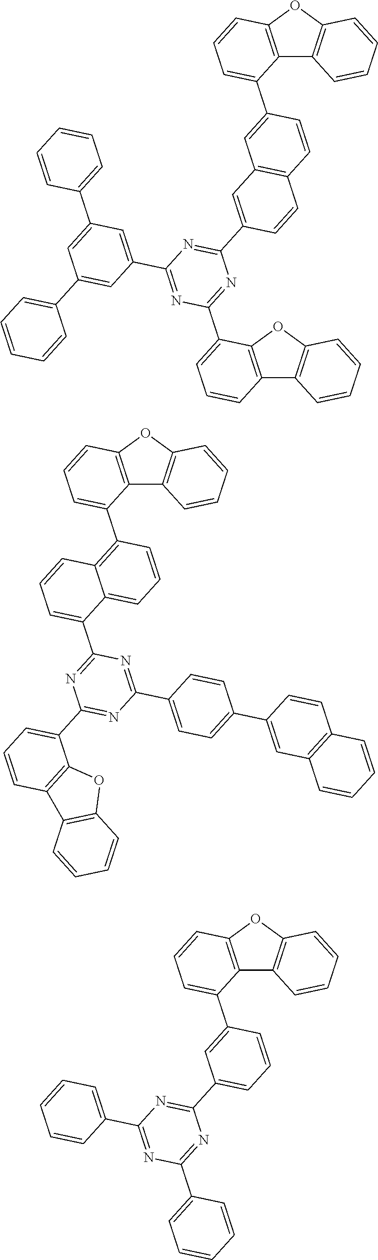 [SMILES: c1ccc(-c2cc(-c3ccccc3)cc(-c3nc(-c4ccc5ccc(-c6cccc7oc8ccccc8c67)cc5c4)nc(-c4cccc5c4oc4ccccc45)n3)c2)cc1 ; c1ccc2cc(-c3ccc(-c4nc(-c5cccc6c(-c7cccc8oc9ccccc9c78)cccc56)nc(-c5cccc6c5oc5ccccc56)n4)cc3)ccc2c1 ; c1ccc(-c2nc(-c3ccccc3)nc(-c3cccc(-c4cccc5oc6ccccc6c45)c3)n2)cc1]

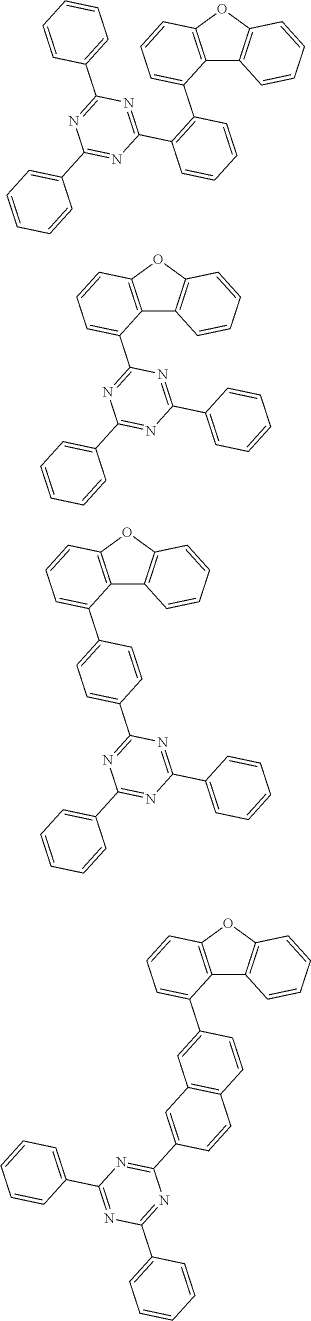

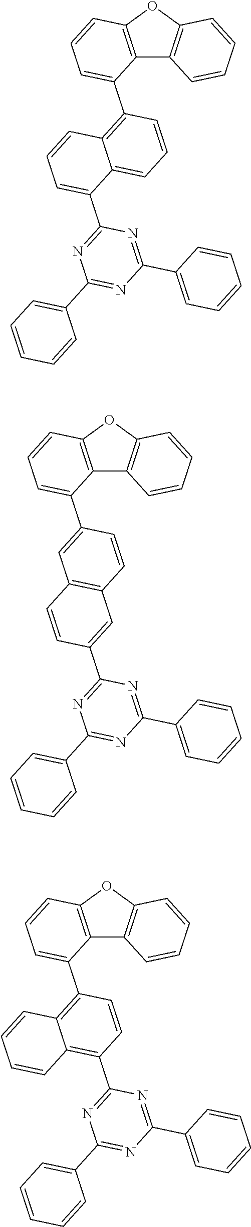 [SMILES: c1ccc(-c2nc(-c3ccccc3)nc(-c3cccc4c(-c5cccc6oc7ccccc7c56)cccc34)n2)cc1 ; c1ccc(-c2nc(-c3ccccc3)nc(-c3ccc4cc(-c5cccc6oc7ccccc7c56)ccc4c3)n2)cc1 ; c1ccc(-c2nc(-c3ccccc3)nc(-c3ccc(-c4cccc5oc6ccccc6c45)c4ccccc34)n2)cc1]

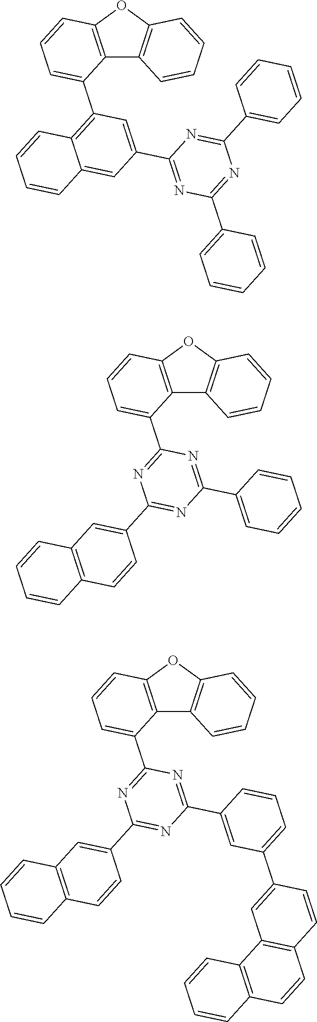 [SMILES: c1ccc(-c2nc(-c3ccccc3)nc(-c3cc(-c4cccc5oc6ccccc6c45)c4ccccc4c3)n2)cc1 ; c1ccc(-c2nc(-c3ccc4ccccc4c3)nc(-c3cccc4oc5ccccc5c34)n2)cc1 ; c1cc(-c2ccc3ccc4ccccc4c3c2)cc(-c2nc(-c3ccc4ccccc4c3)nc(-c3cccc4oc5ccccc5c34)n2)c1]

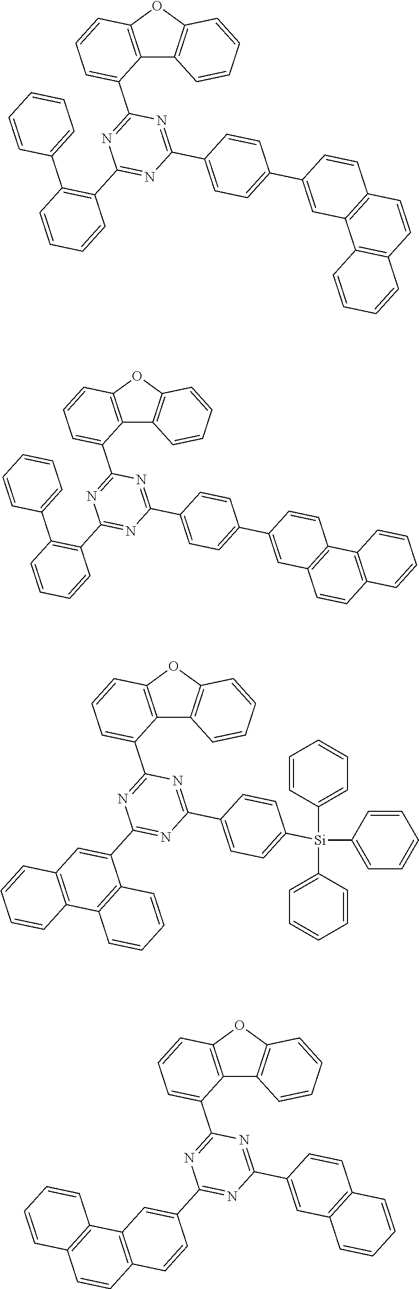 [SMILES: c1ccc(-c2ccccc2-c2nc(-c3ccc(-c4ccc5ccc6ccccc6c5c4)cc3)nc(-c3cccc4oc5ccccc5c34)n2)cc1 ; c1ccc(-c2ccccc2-c2nc(-c3ccc(-c4ccc5c(ccc6ccccc65)c4)cc3)nc(-c3cccc4oc5ccccc5c34)n2)cc1 ; c1ccc([Si](c2ccccc2)(c2ccccc2)c2ccc(-c3nc(-c4cc5ccccc5c5ccccc45)nc(-c4cccc5oc6ccccc6c45)n3)cc2)cc1 ; c1ccc2cc(-c3nc(-c4ccc5ccc6ccccc6c5c4)nc(-c4cccc5oc6ccccc6c45)n3)ccc2c1]

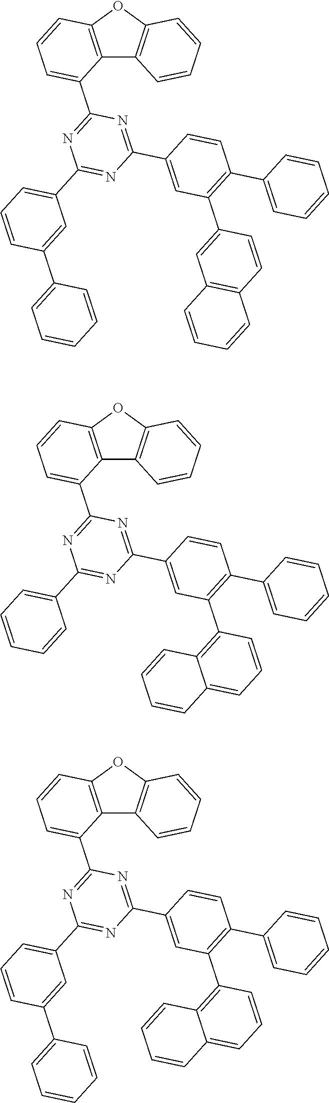 [SMILES: c1ccc(-c2cccc(-c3nc(-c4ccc(-c5ccccc5)c(-c5ccc6ccccc6c5)c4)nc(-c4cccc5oc6ccccc6c45)n3)c2)cc1 ; c1ccc(-c2nc(-c3ccc(-c4ccccc4)c(-c4cccc5ccccc45)c3)nc(-c3cccc4oc5ccccc5c34)n2)cc1 ; c1ccc(-c2cccc(-c3nc(-c4ccc(-c5ccccc5)c(-c5cccc6ccccc56)c4)nc(-c4cccc5oc6ccccc6c45)n3)c2)cc1]

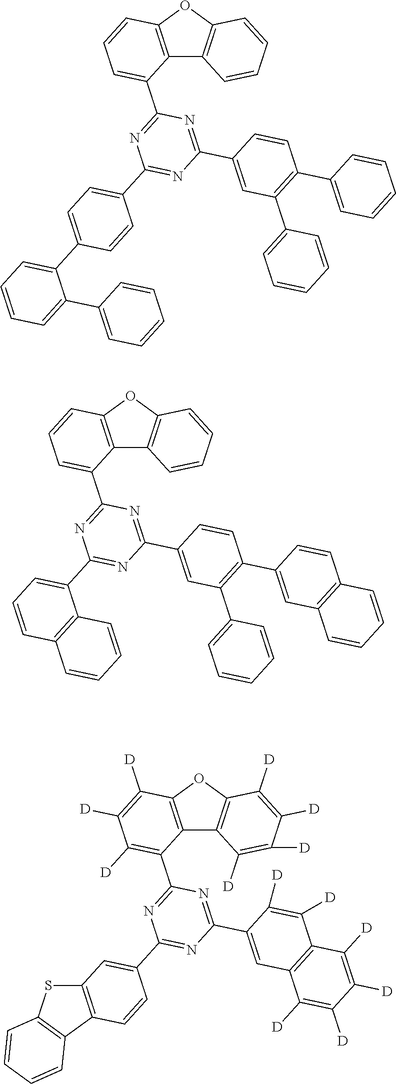 [SMILES: c1ccc(-c2ccccc2-c2ccc(-c3nc(-c4ccc(-c5ccccc5)c(-c5ccccc5)c4)nc(-c4cccc5oc6ccccc6c45)n3)cc2)cc1 ; c1ccc(-c2cc(-c3nc(-c4cccc5ccccc45)nc(-c4cccc5oc6ccccc6c45)n3)ccc2-c2ccc3ccccc3c2)cc1 ; [2H]c1c([2H])c([2H])c2c([2H])c([2H])c(-c3nc(-c4ccc5c(c4)sc4ccccc45)nc(-c4c([2H])c([2H])c([2H])c5oc6c([2H])c([2H])c([2H])c([2H])c6c45)n3)cc2c1[2H]]

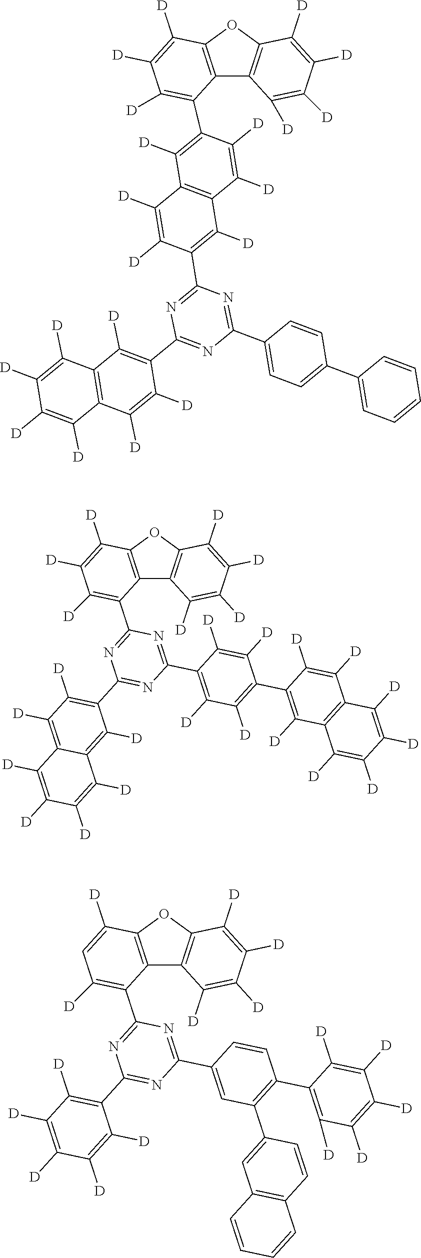

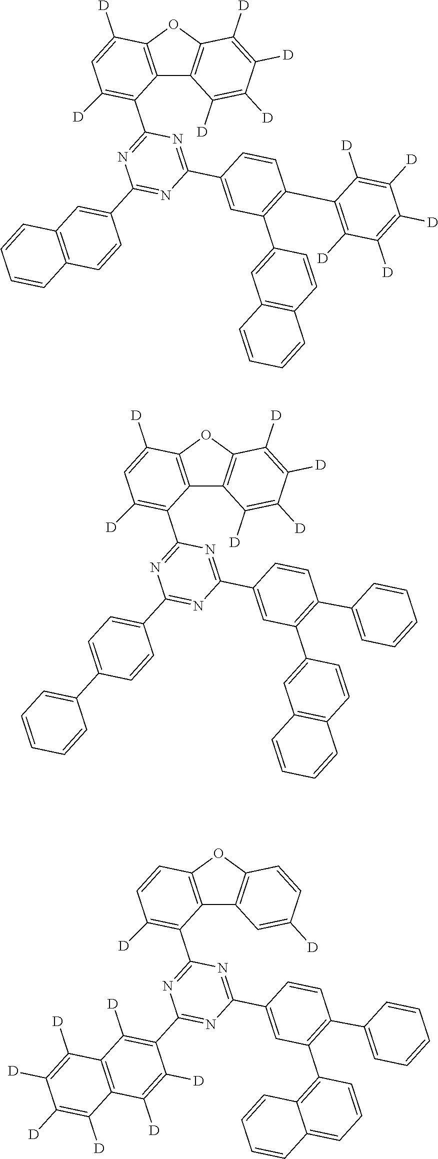 [SMILES: [2H]c1cc([2H])c2oc3c([2H])c([2H])c([2H])c([2H])c3c2c1-c1nc(-c2ccc(-c3c([2H])c([2H])c([2H])c([2H])c3[2H])c(-c3ccc4ccccc4c3)c2)nc(-c2ccc3ccccc3c2)n1 ; [2H]c1cc([2H])c2oc3c([2H])c([2H])c([2H])c([2H])c3c2c1-c1nc(-c2ccc(-c3ccccc3)cc2)nc(-c2ccc(-c3ccccc3)c(-c3ccc4ccccc4c3)c2)n1 ; [2H]c1ccc2oc3ccc([2H])c(-c4nc(-c5ccc(-c6ccccc6)c(-c6cccc7ccccc67)c5)nc(-c5c([2H])c([2H])c6c([2H])c([2H])c([2H])c([2H])c6c5[2H])n4)c3c2c1]

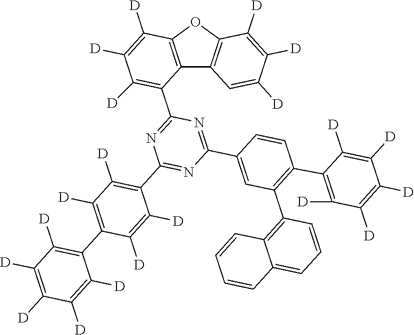 [SMILES: [2H]c1cc2c(oc3c([2H])c([2H])c([2H])c(-c4nc(-c5ccc(-c6c([2H])c([2H])c([2H])c([2H])c6[2H])c(-c6cccc7ccccc67)c5)nc(-c5c([2H])c([2H])c(-c6c([2H])c([2H])c([2H])c([2H])c6[2H])c([2H])c5[2H])n4)c32)c([2H])c1[2H]]

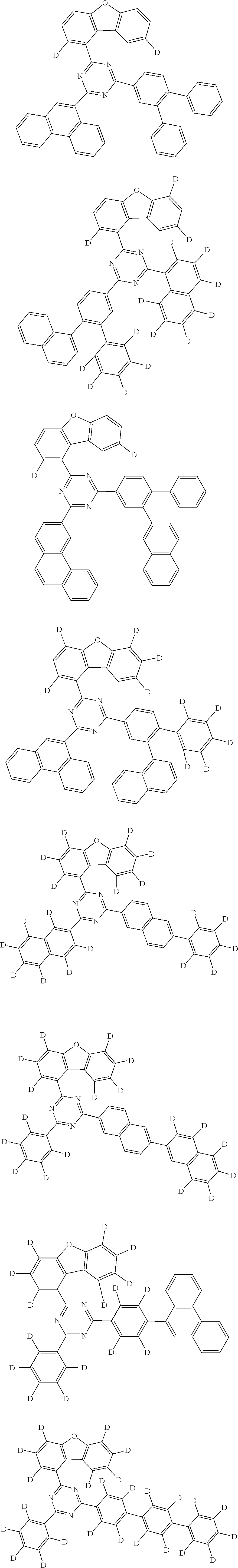

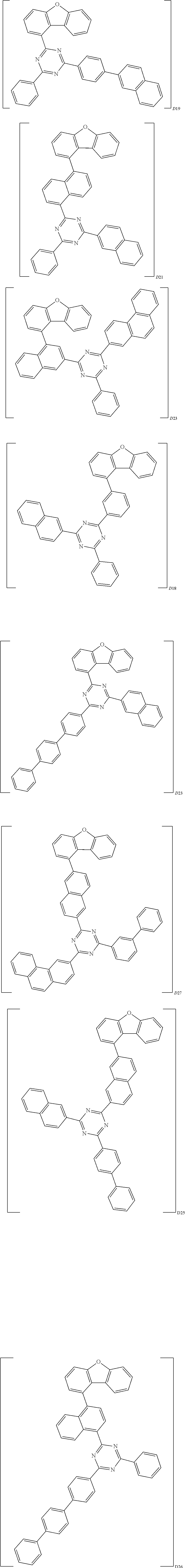

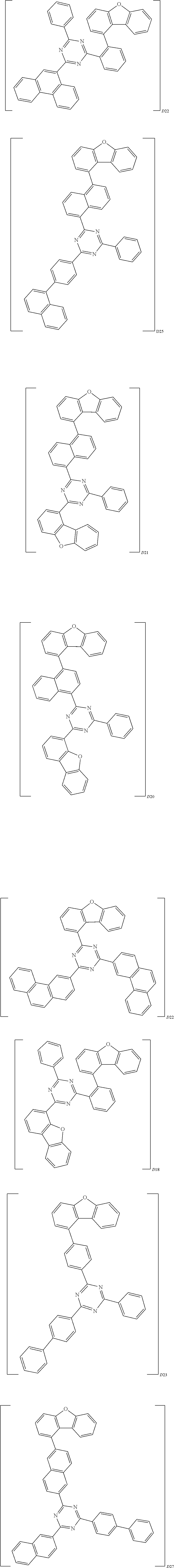

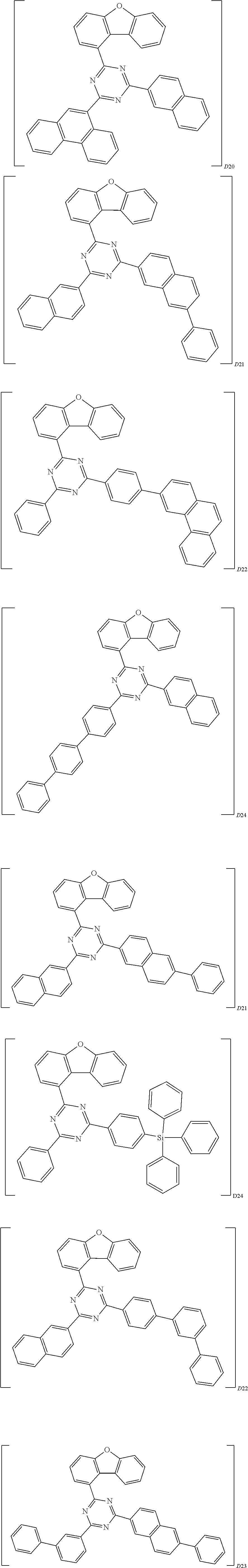

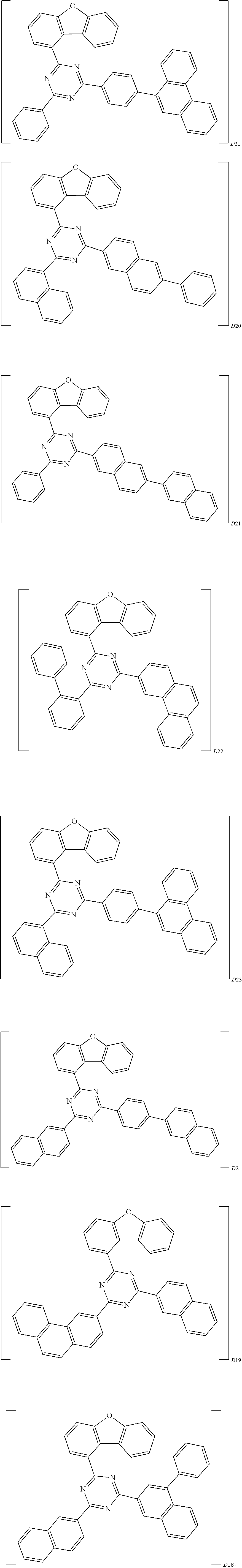

8. The organic light emitting device of claim 1, wherein:
the second compound is represented by the following Chemical Formula 2-1: 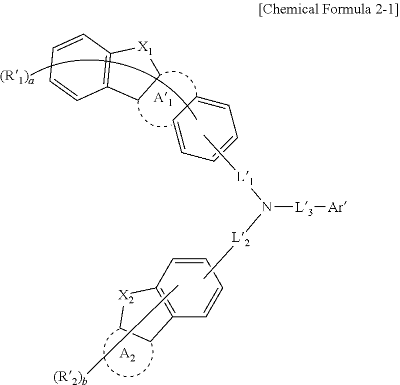
wherein in Chemical Formula 2-1:
A′₁ is a benzene ring fused with adjacent pentagonal and benzene rings; and
X₁, X₂, A₂, L′₁ to L′₃, Ar′, R′₁, R′₂, a and b are as defined in claim 1.

9. The organic light emitting device of claim 1, wherein:
both X₁ and X₂ are O; or
both X₁ and X₂ are S; or
X₁ is O, and X₂ is S; or
X₁ is S, and X₂ is O.

10. The organic light emitting device of claim 1, wherein: 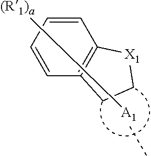
is any one of the substituents represented by the following Chemical Formulas 2a to 2l: 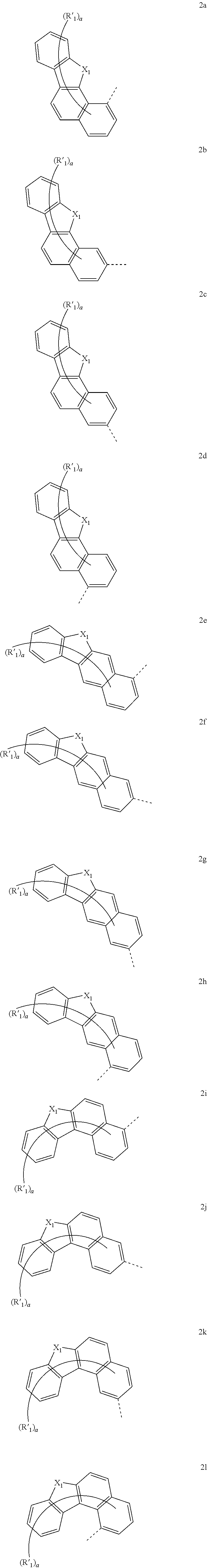
wherein in Chemical Formulas 2a to 2l:
X₁, R′₁ and a are as defined in claim 1.

11. The organic light emitting device of claim 1, wherein: 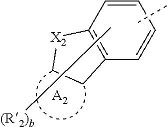
is any one of the substituents represented by the following Chemical Formulas 3a to 3l: 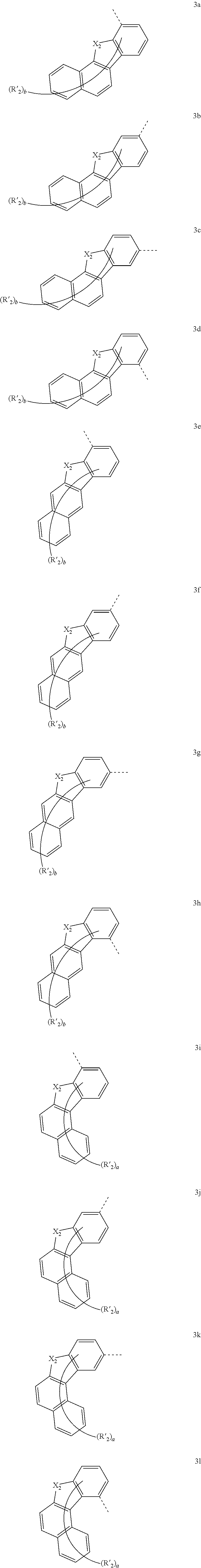
wherein in Chemical Formulas 3a to 3l,
X₂, R′₂ and b are as defined in claim 1.

12. The organic light emitting device of claim 1, wherein:
L′₁ and L′₂ are each independently a single bond, or a substituted or unsubstituted C₆₋₁₂ arylene.

13. The organic light emitting device of claim 1, wherein:
L′₃ is a single bond, or a substituted or unsubstituted phenylene.

14. The organic light emitting device of claim 1, wherein:
Ar′ is phenyl, biphenylyl, terphenylyl, naphthyl, phenanthryl, triphenylenyl, dibenzofuranyl, dibenzothiophenyl, benzonaphthofuranyl, or benzonaphthothiophenyl,
wherein the Ar′ is unsubstituted, or substituted with one or more substituents selected from the group consisting of deuterium, halogen, cyano, phenyl substituted or unsubstituted with deuterium, biphenylyl substituted or unsubstituted with deuterium, and naphthyl substituted or unsubstituted with deuterium.

15. The organic light emitting device of claim 1, wherein:
both R′₁ and R′₂ are hydrogen, or both are deuterium.

16. The organic light emitting device of claim 1, wherein:
the second compound is represented by any one of the following Chemical Formulas 2-1 to 2-9: 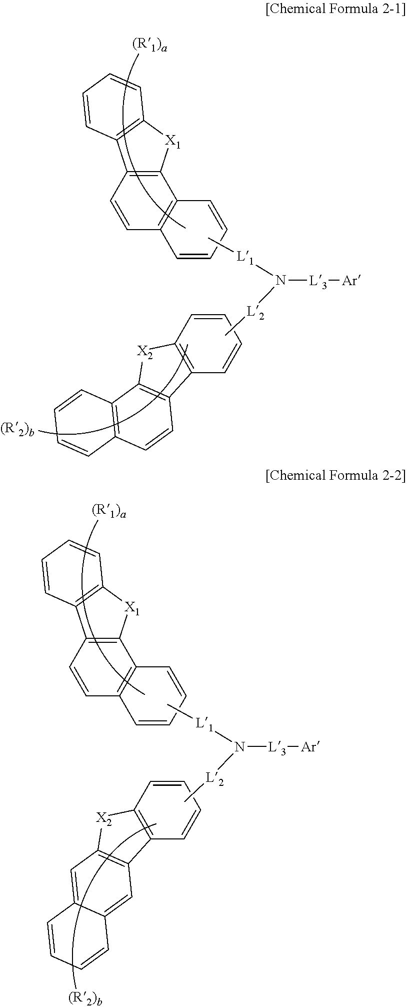 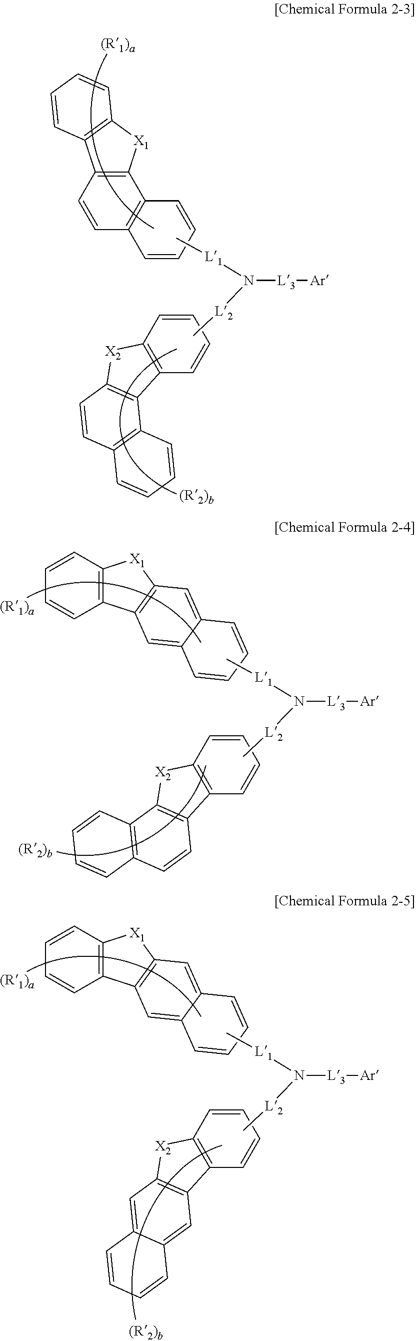 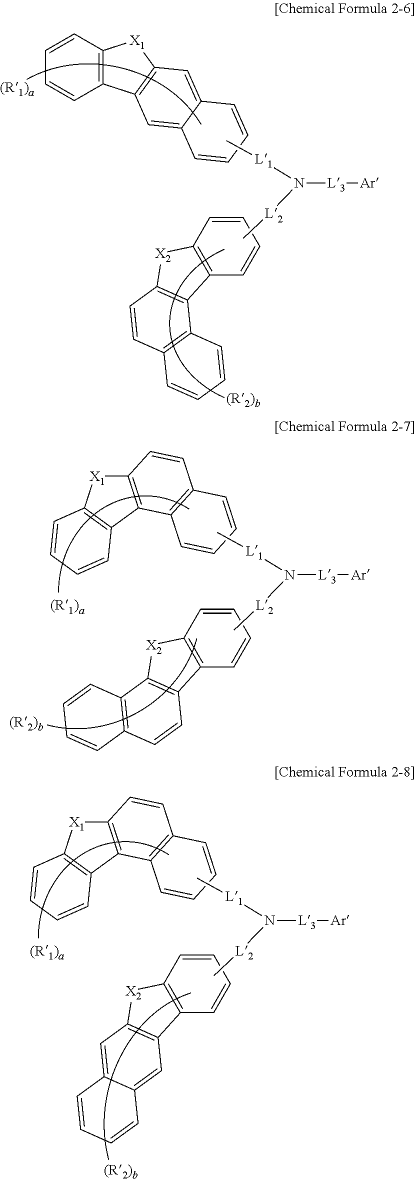 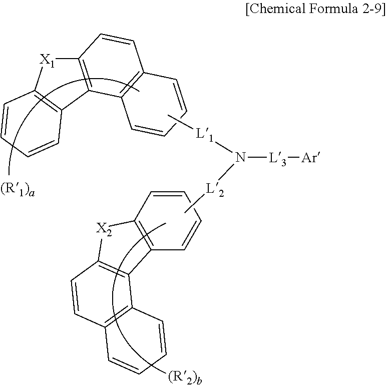
wherein in Chemical Formulas 2-1 to 2-9,
X₁, X₂, L′₁ to L′₃, Ar′, R′₁, R′₂, a and b are as defined in claim 1.

17. The organic light emitting device of claim 1, wherein:
the second compound is any one selected from among the following compounds: 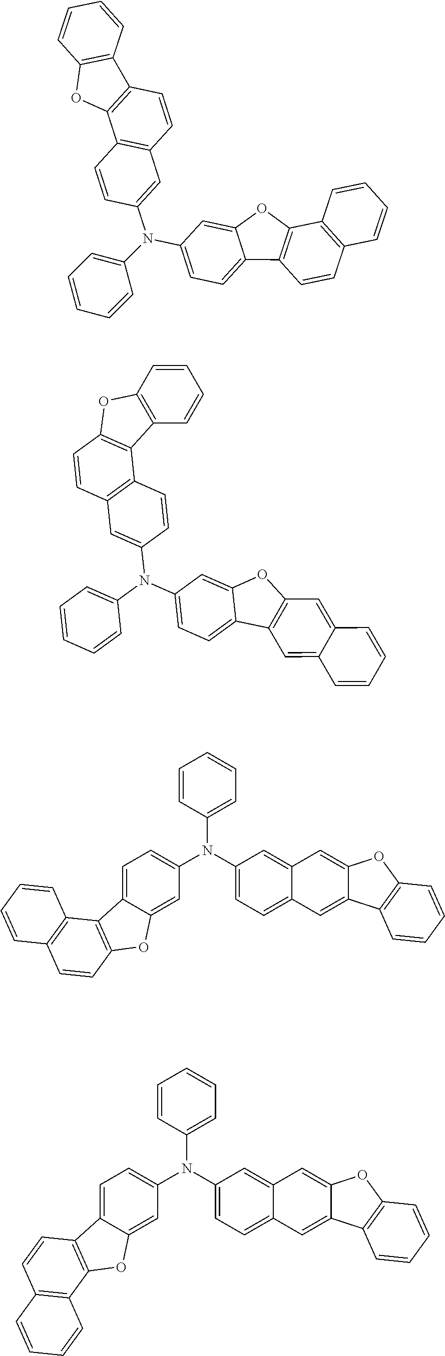 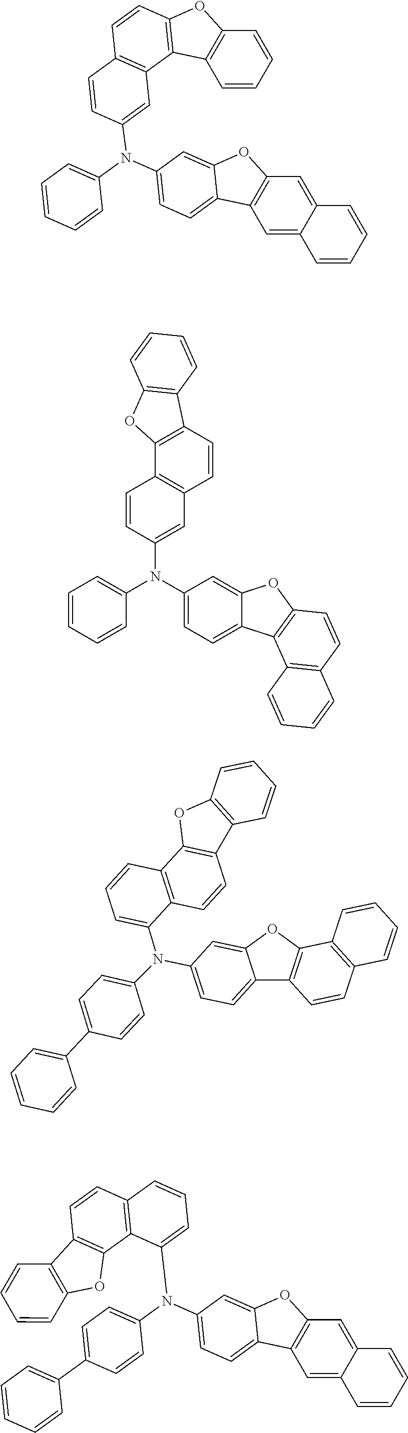 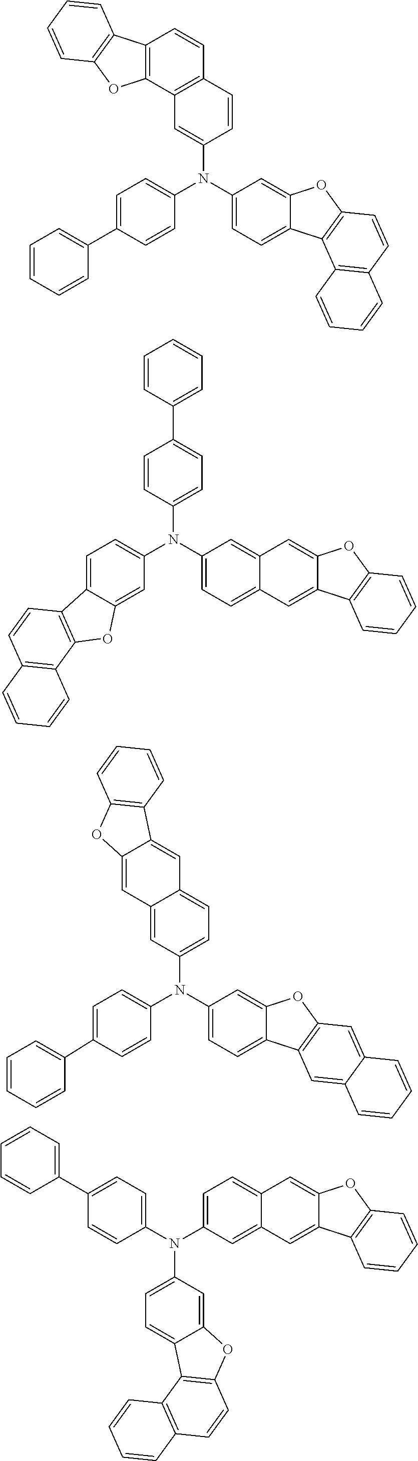 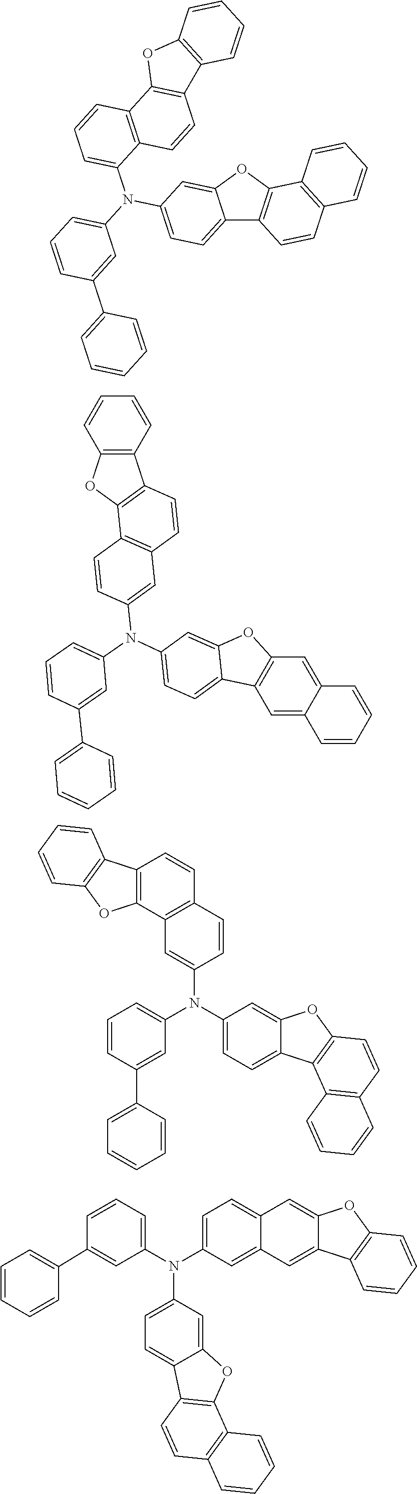 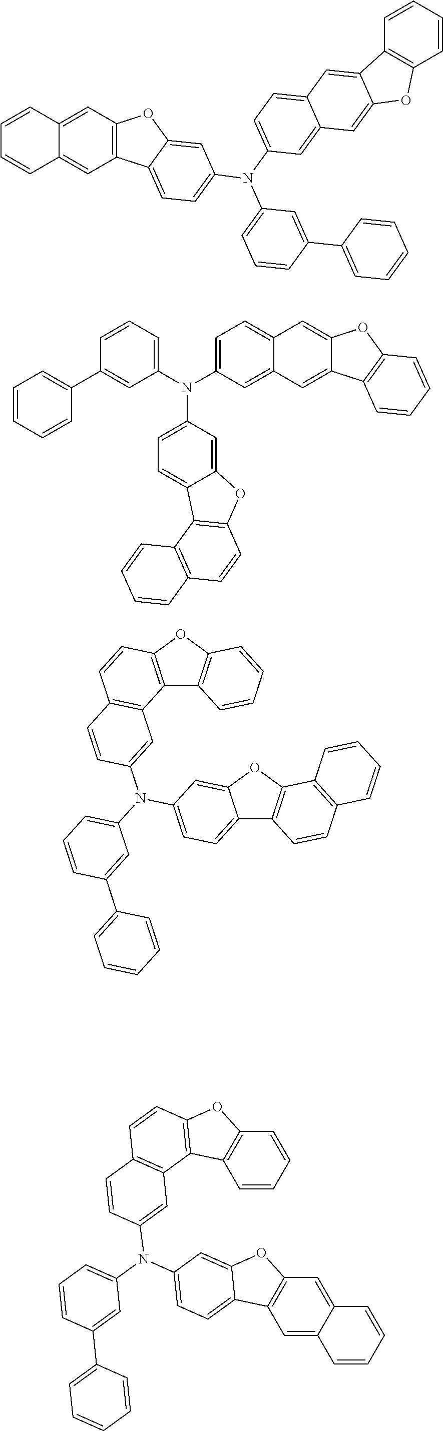 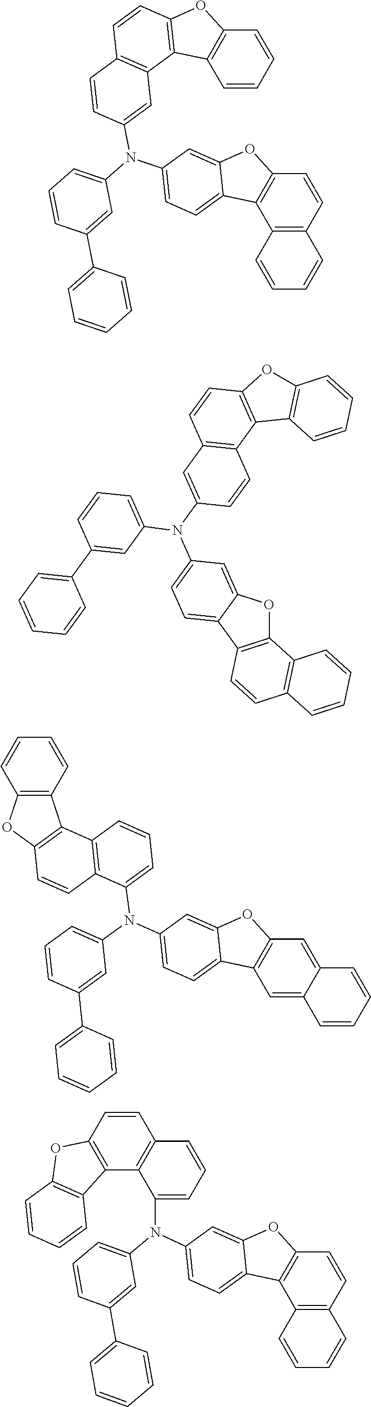 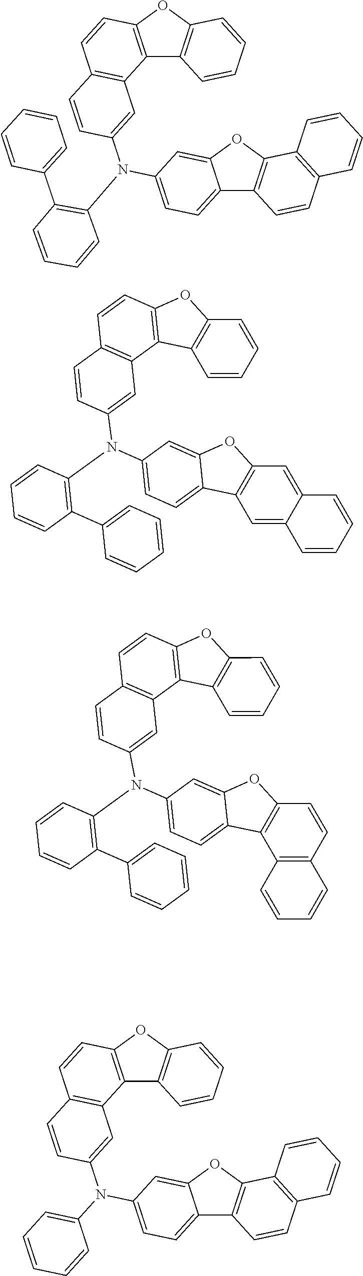 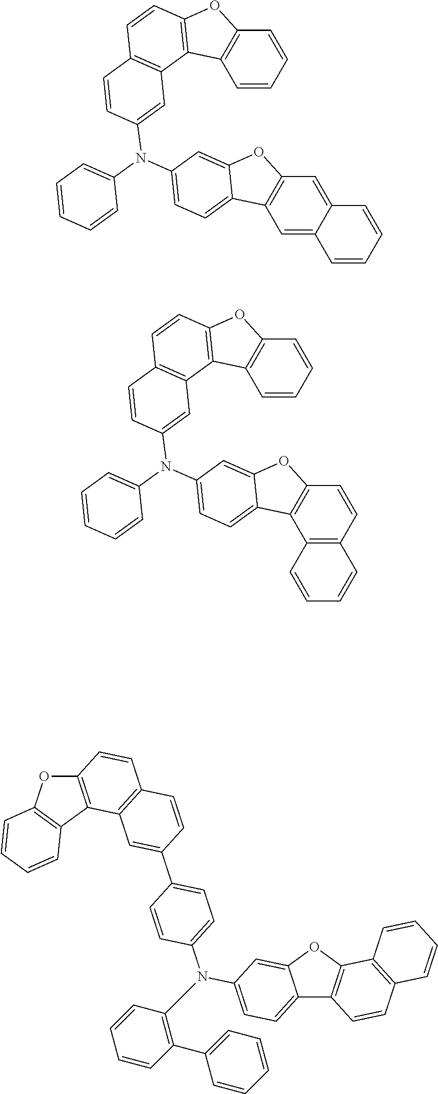 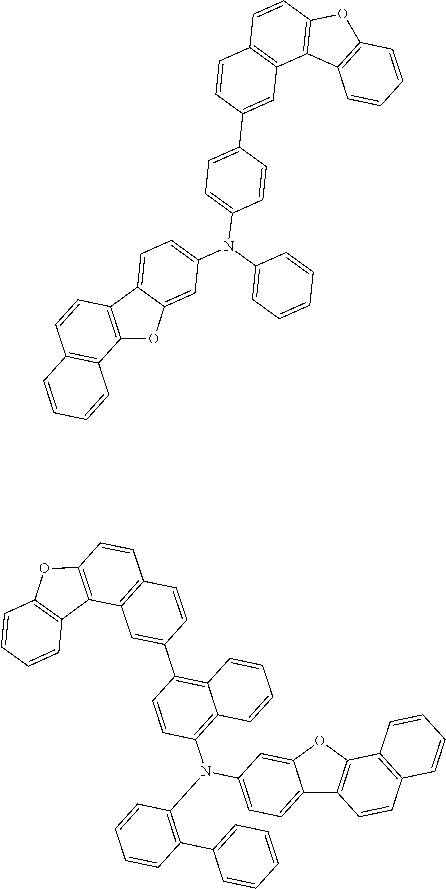 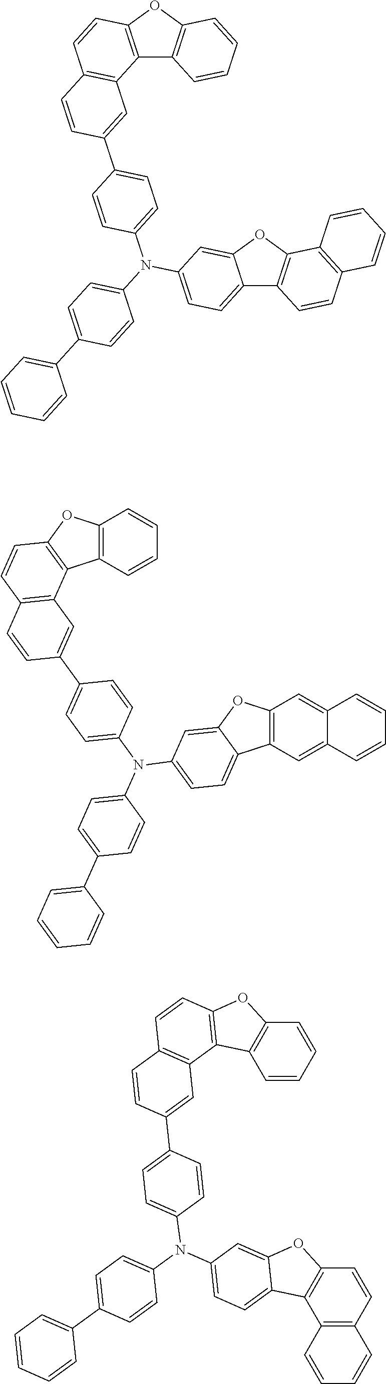 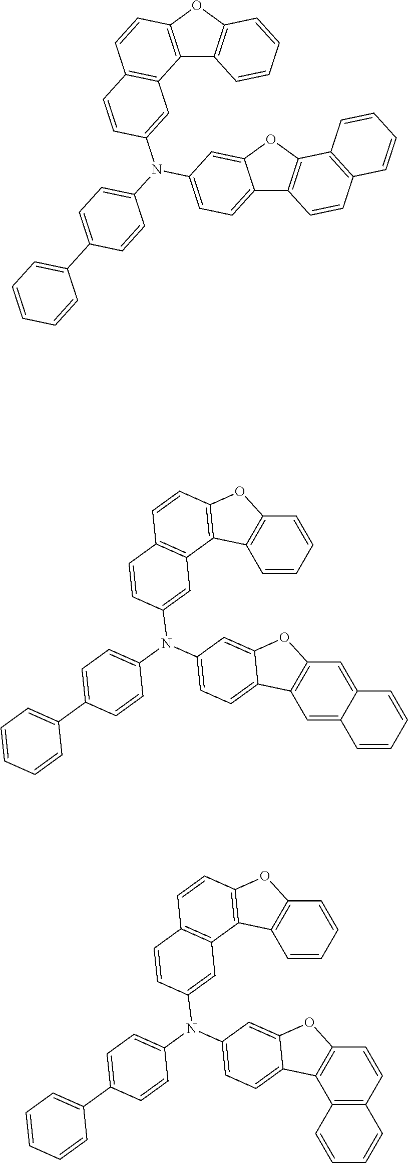 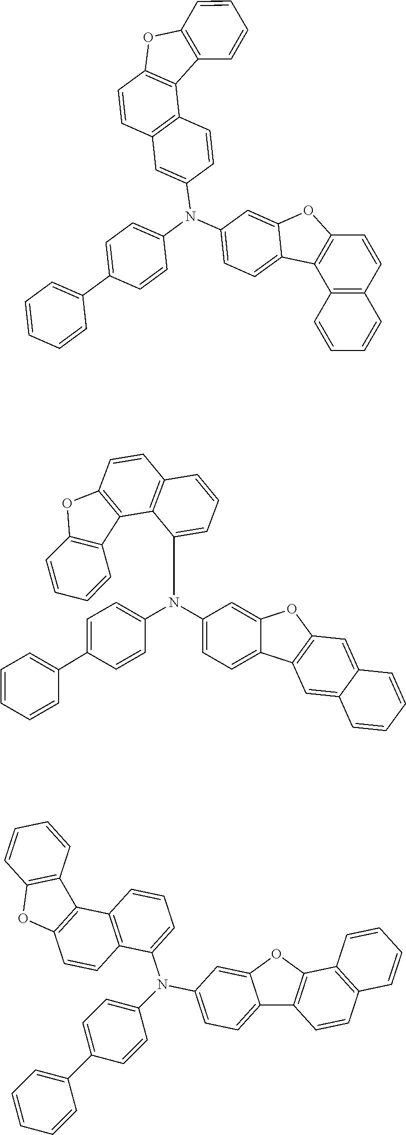 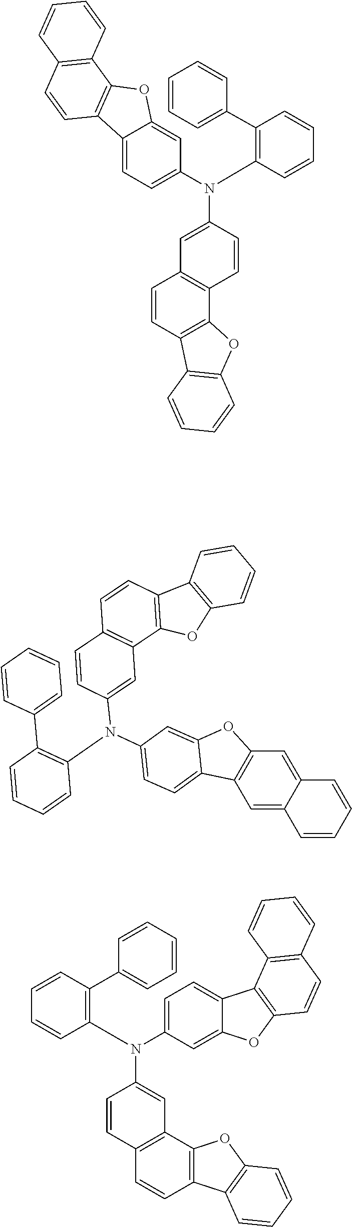 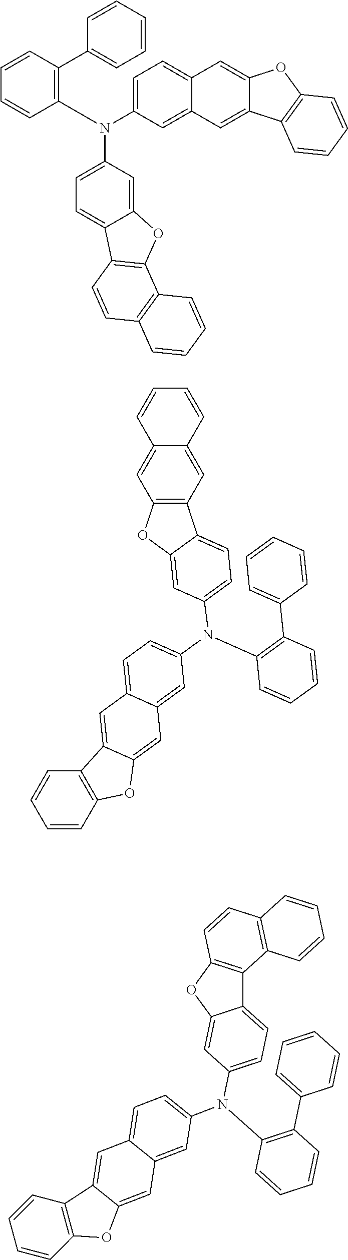 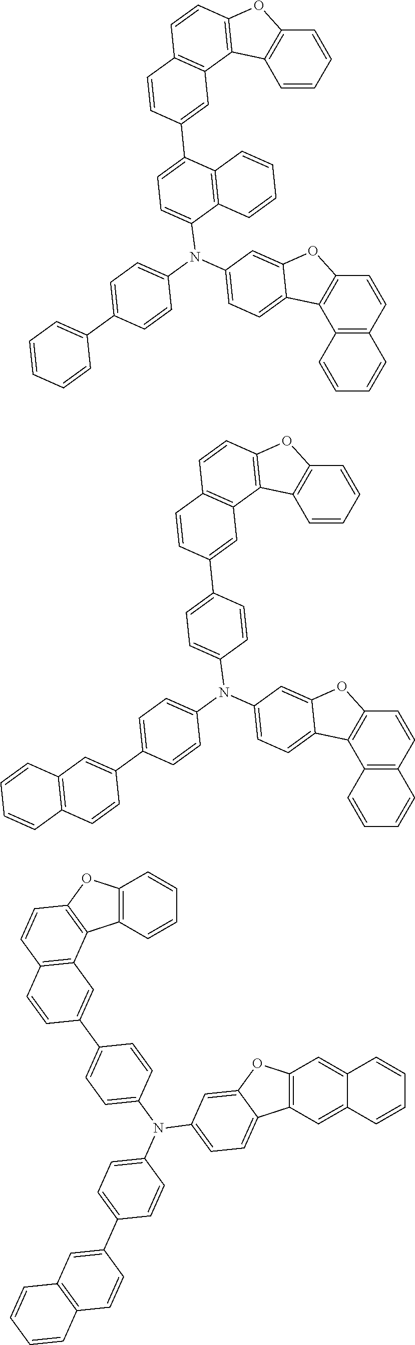 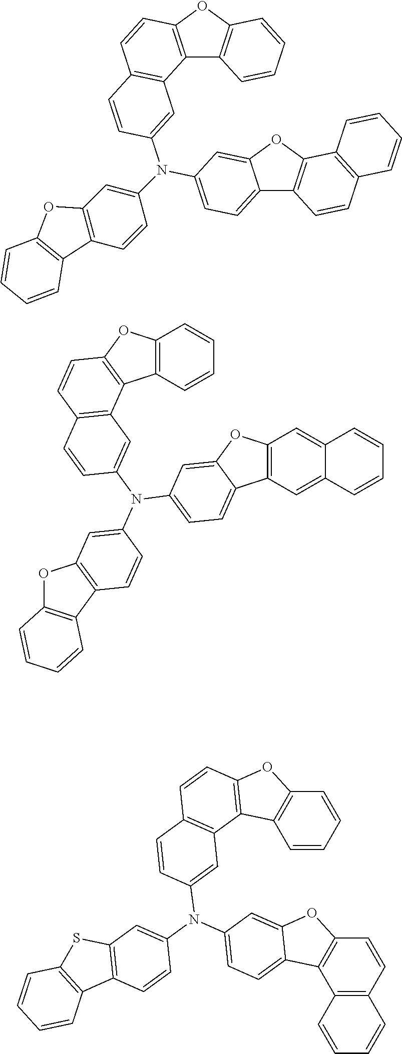 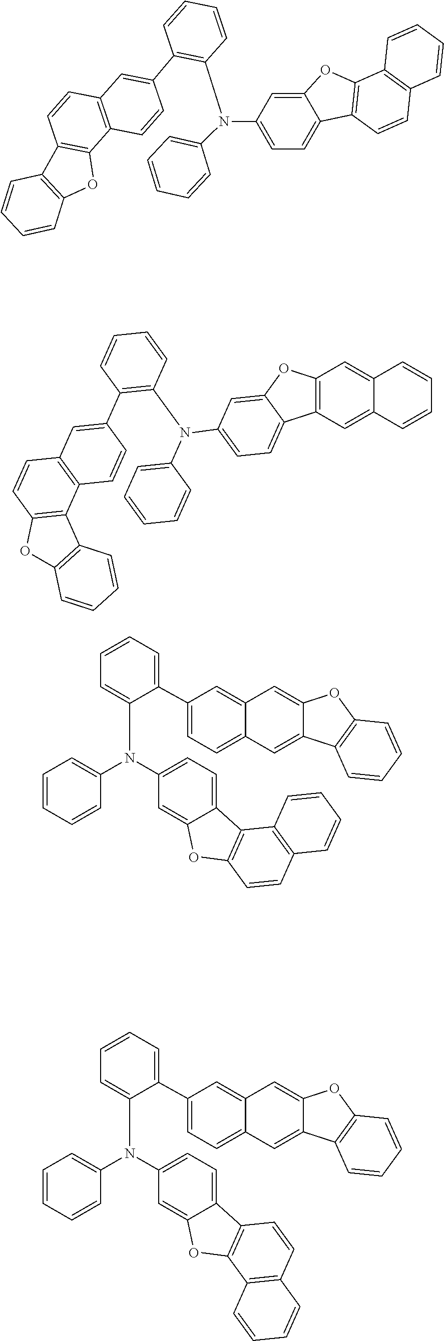 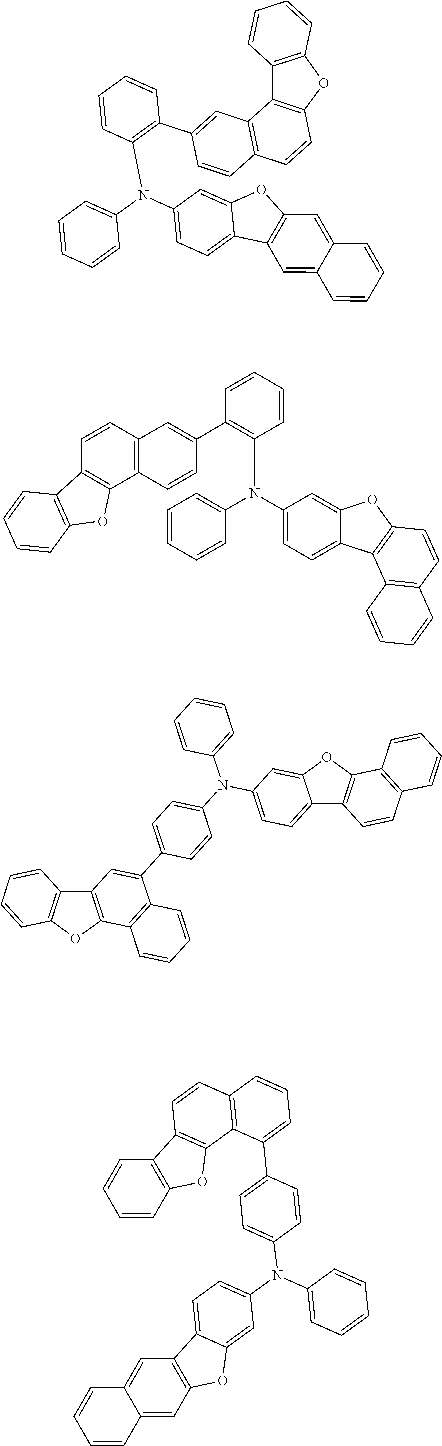 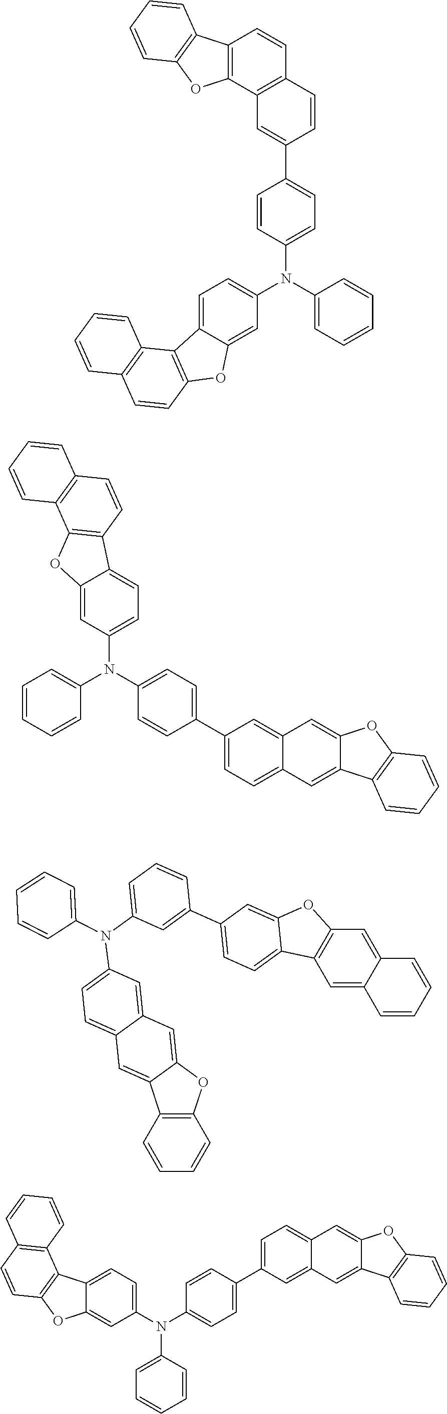 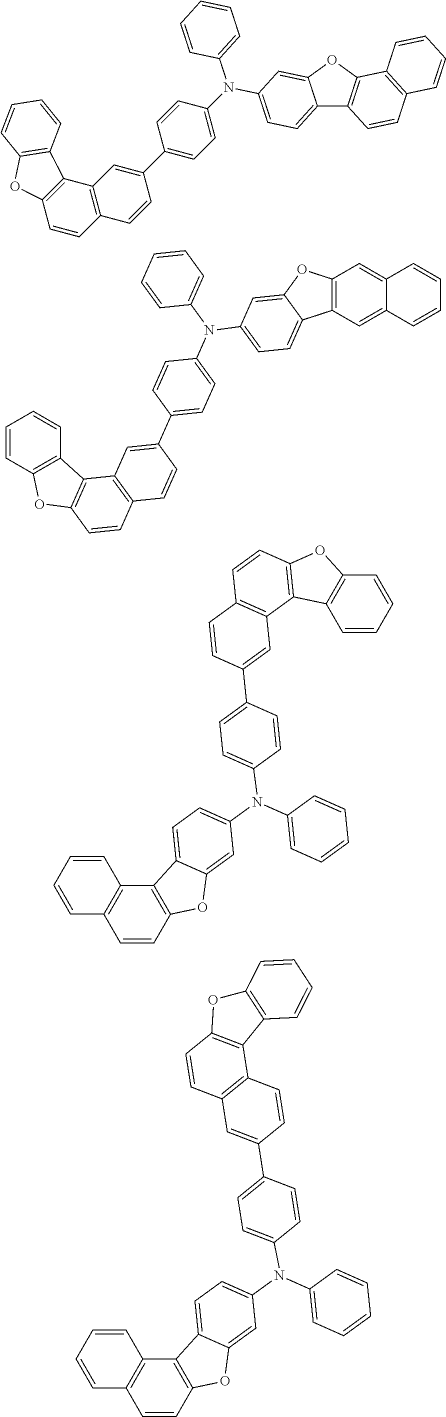 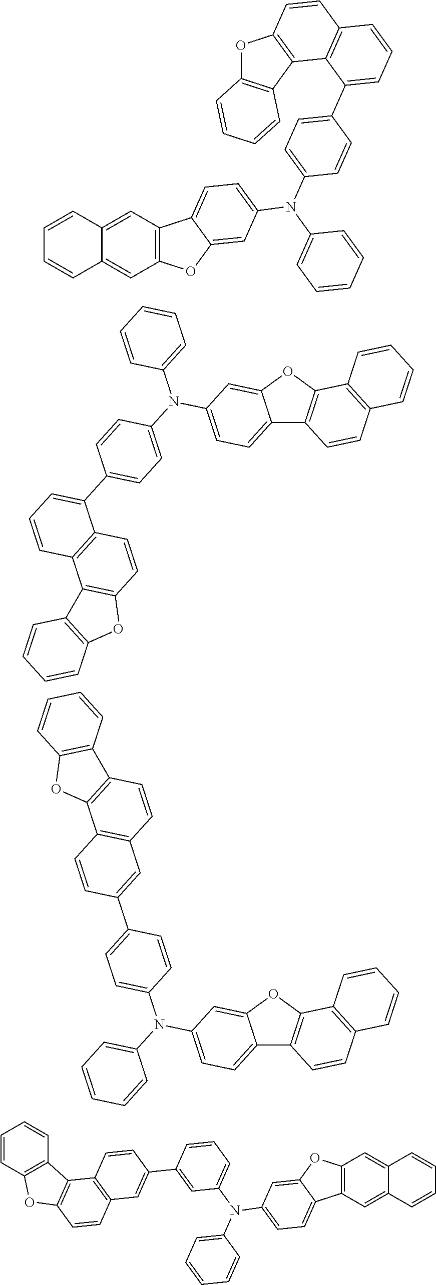 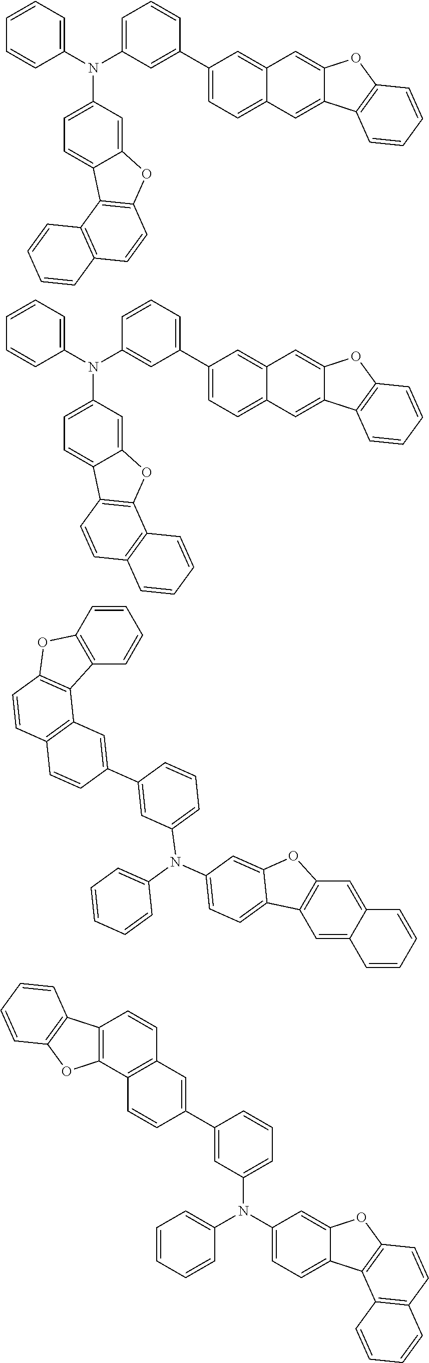 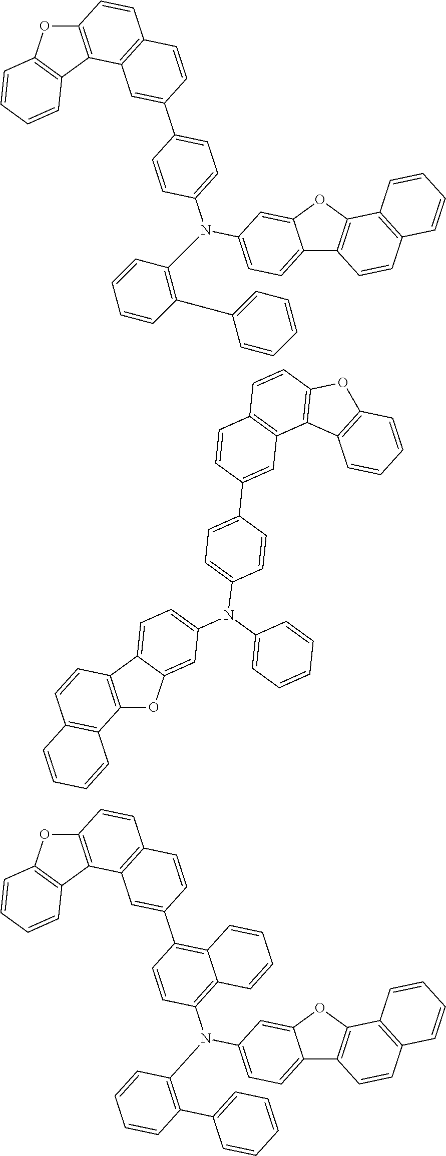 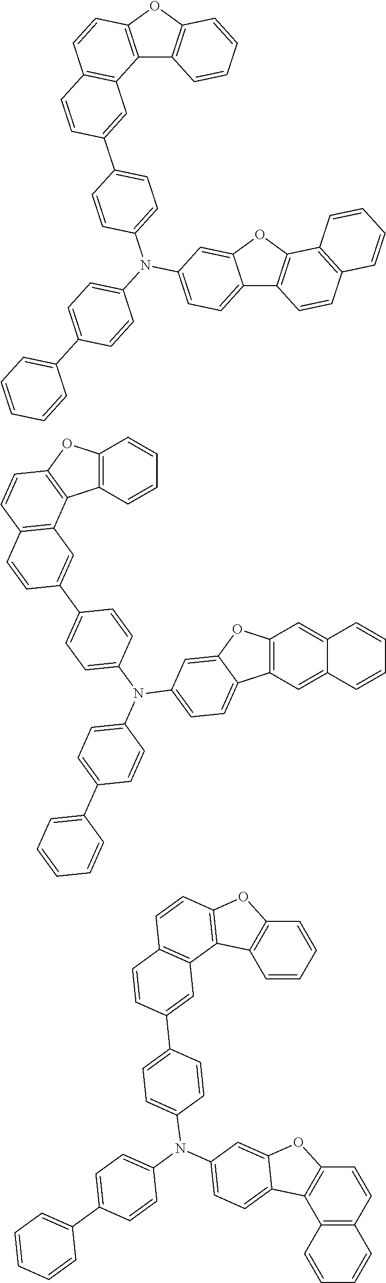 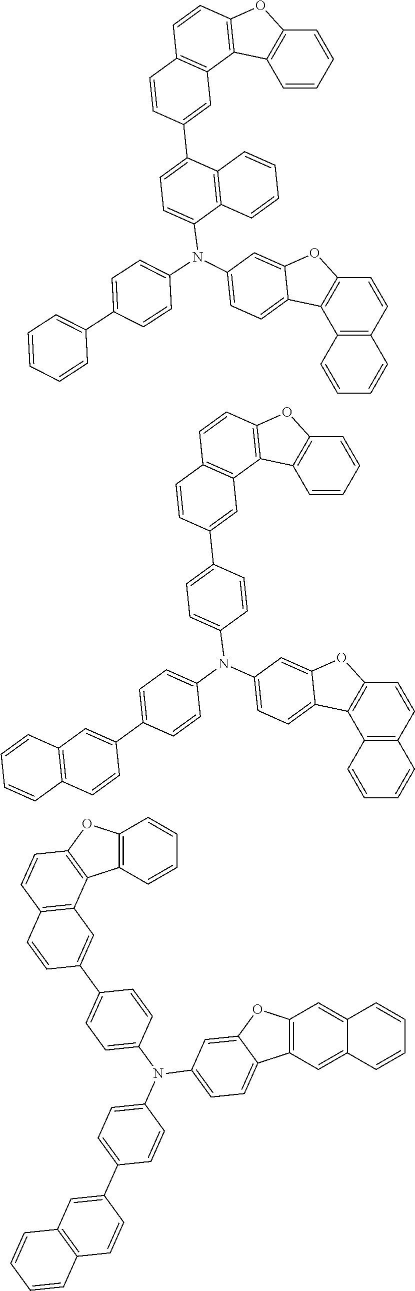 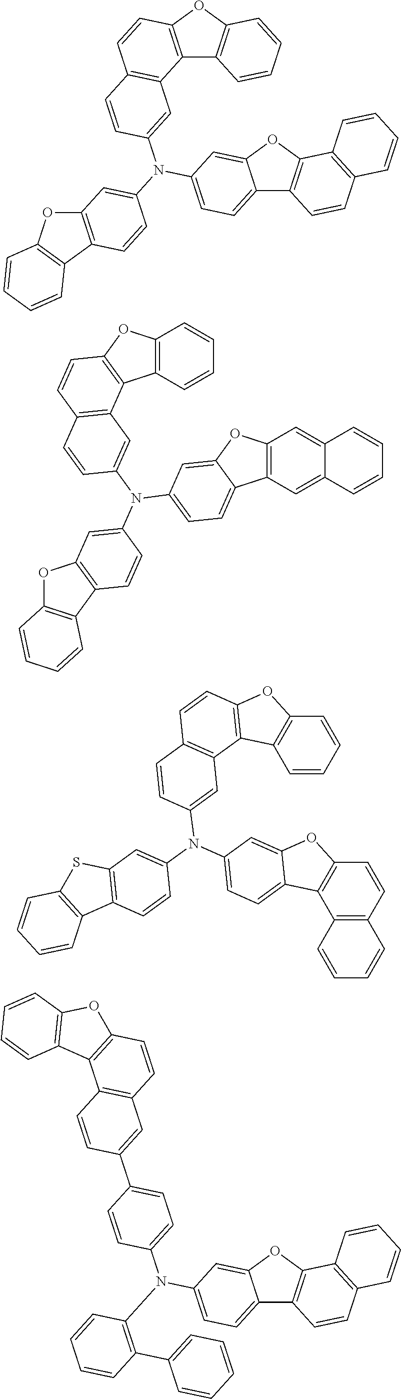 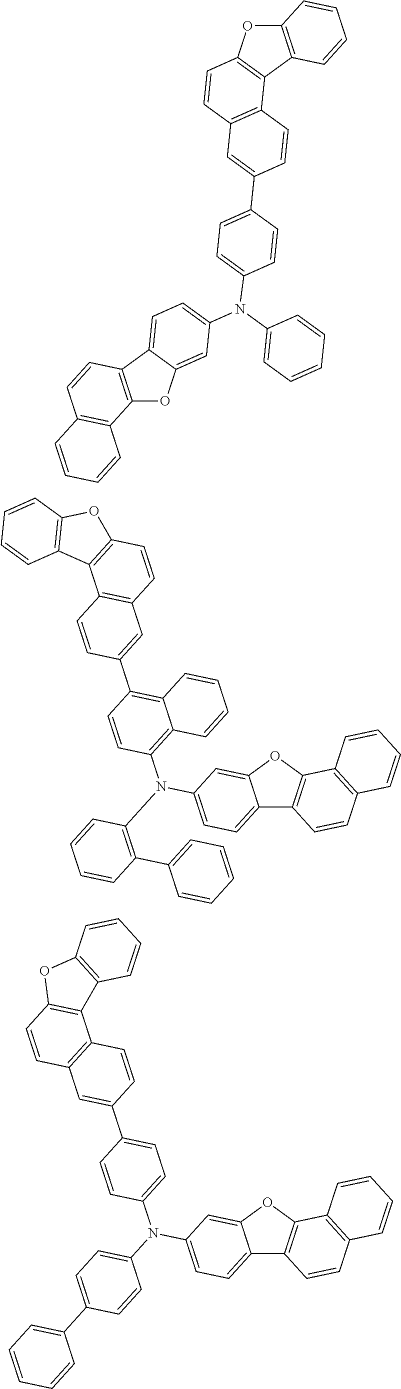 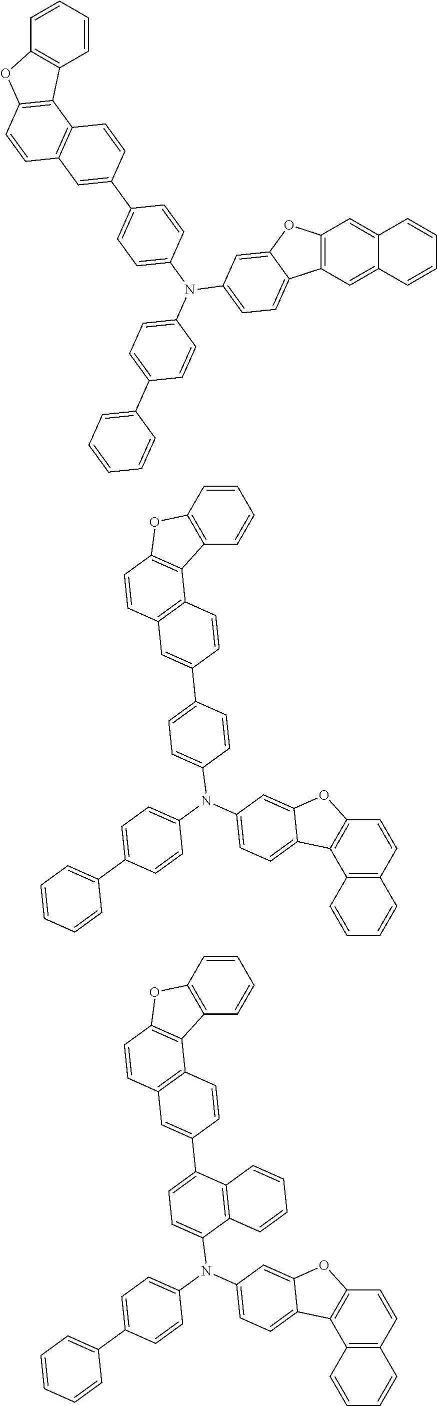 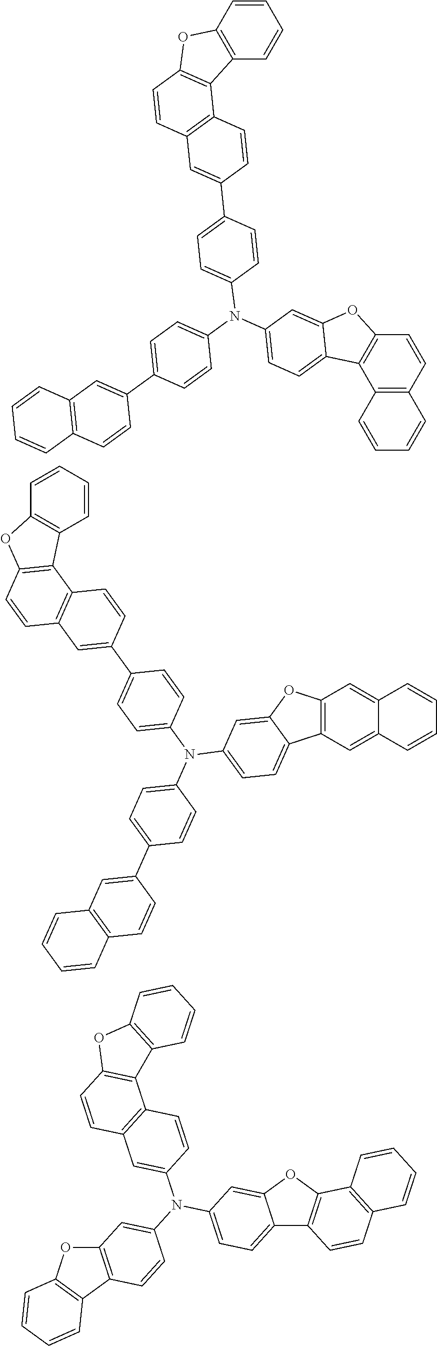 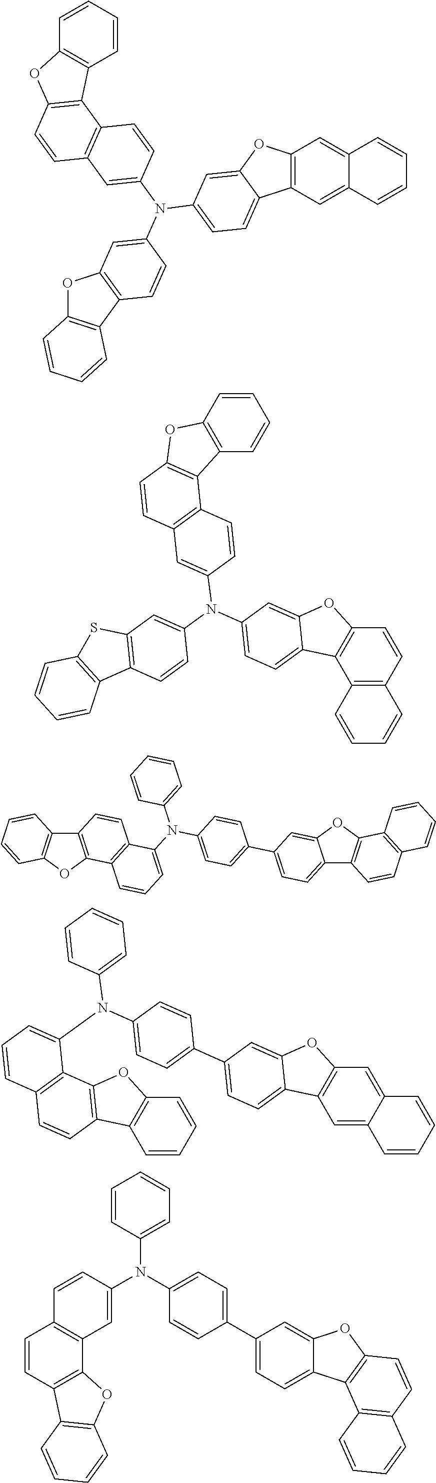 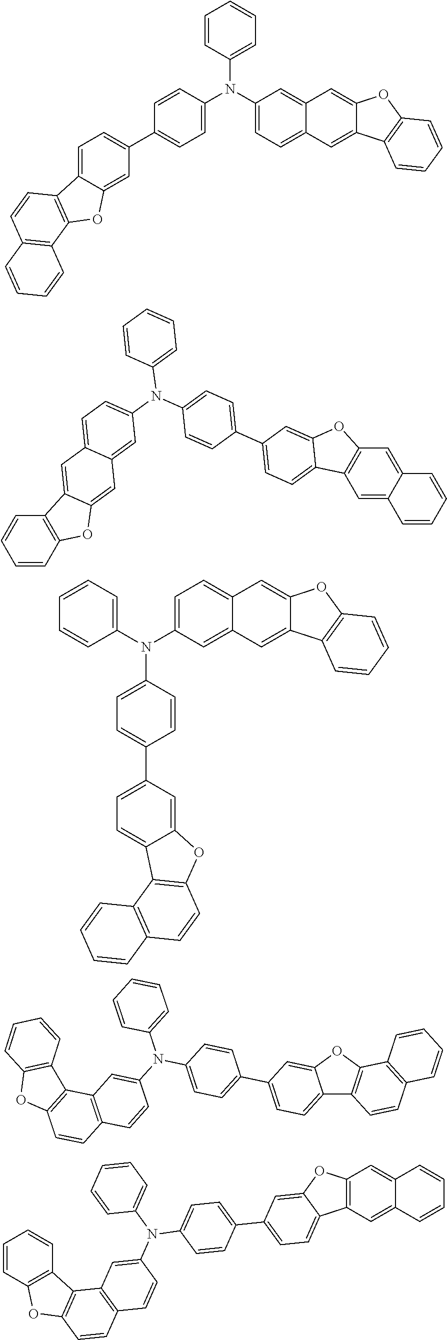 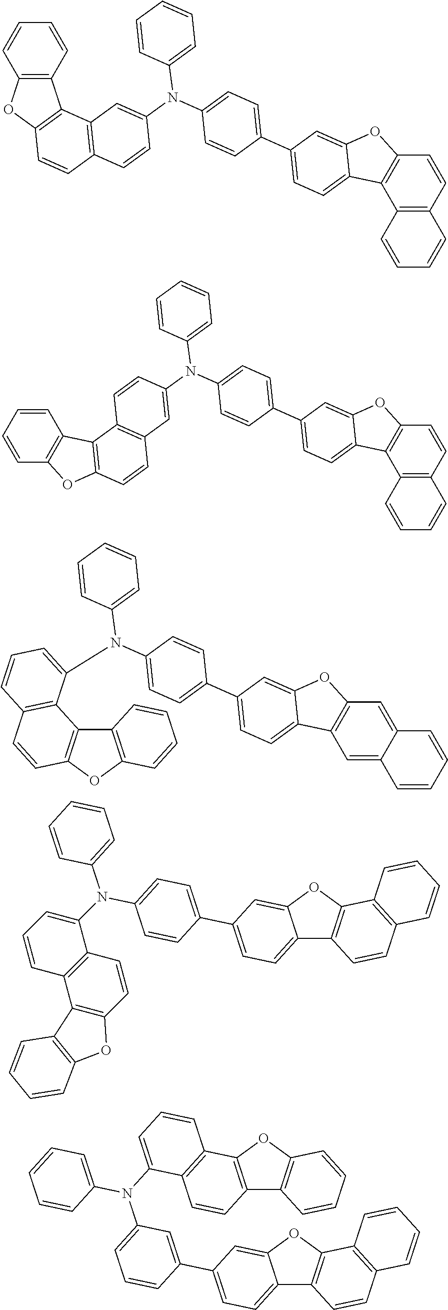 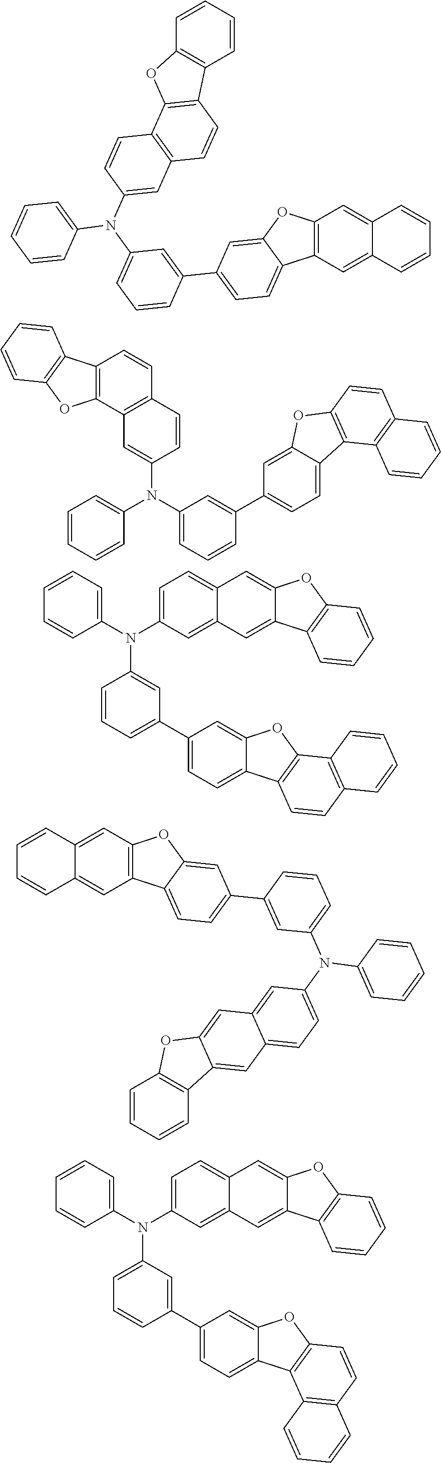 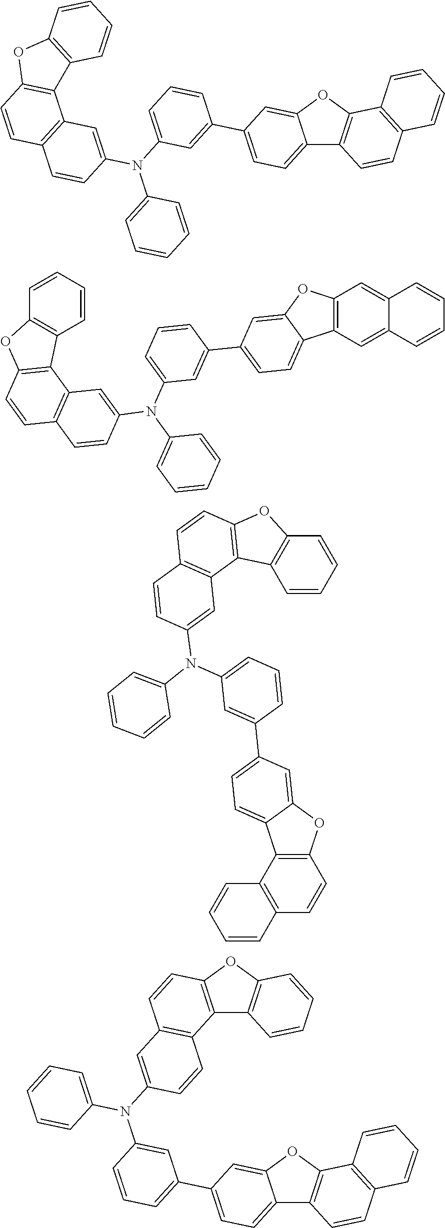 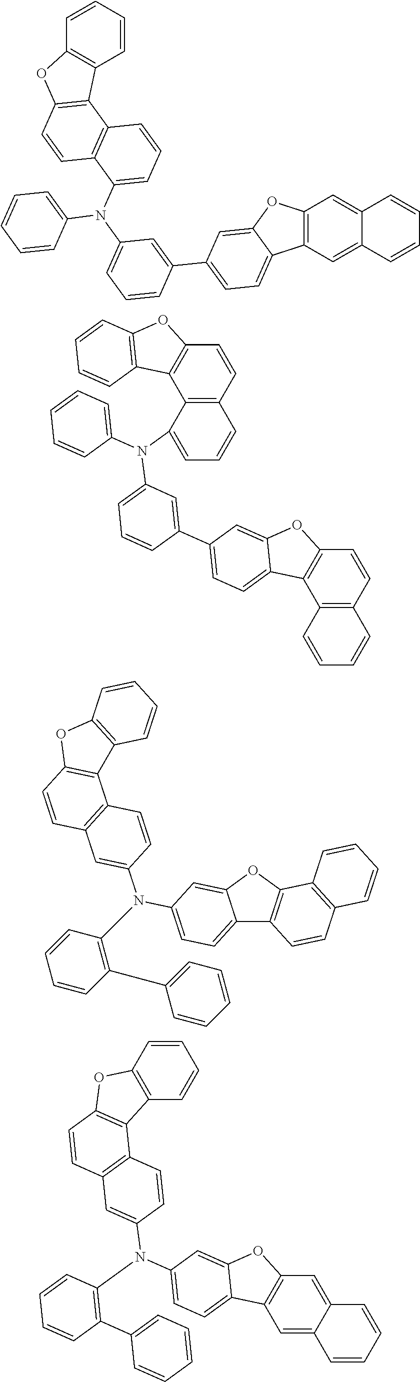 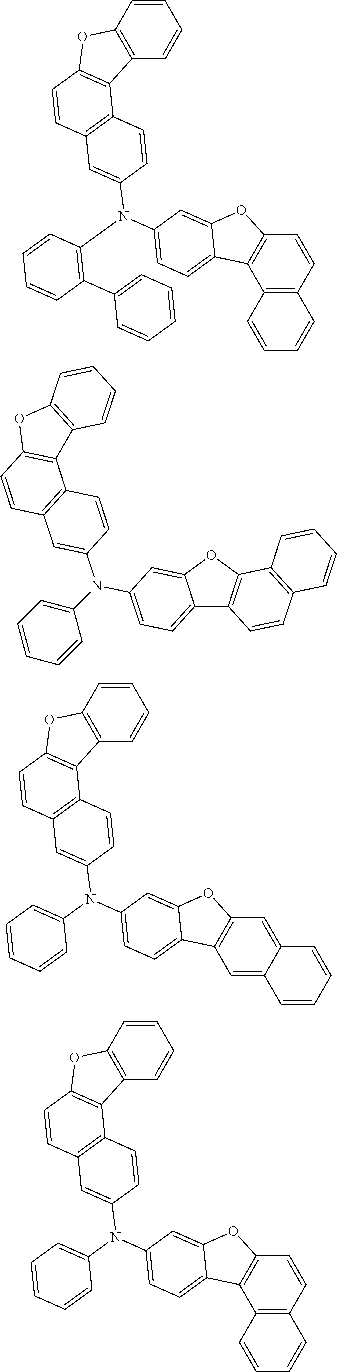 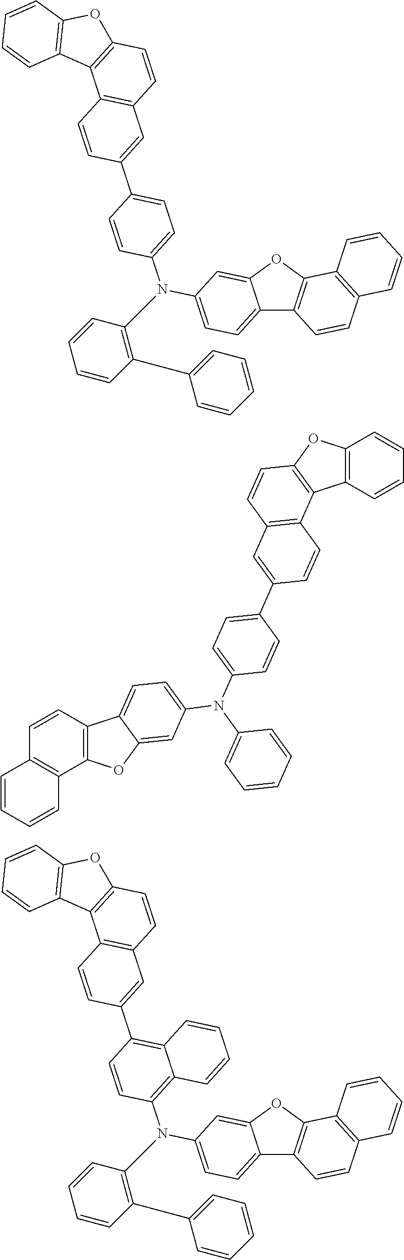 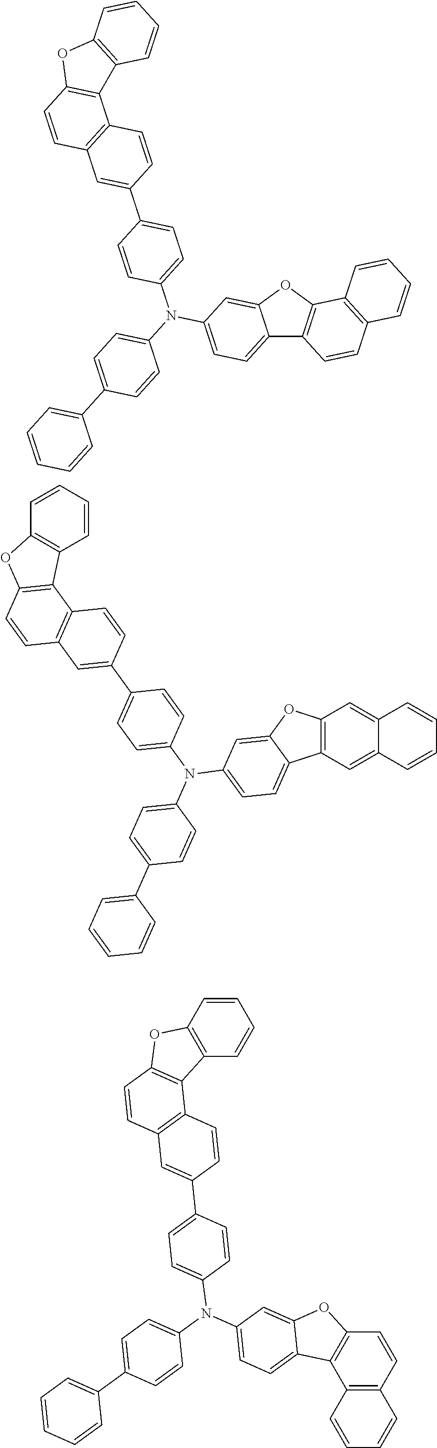 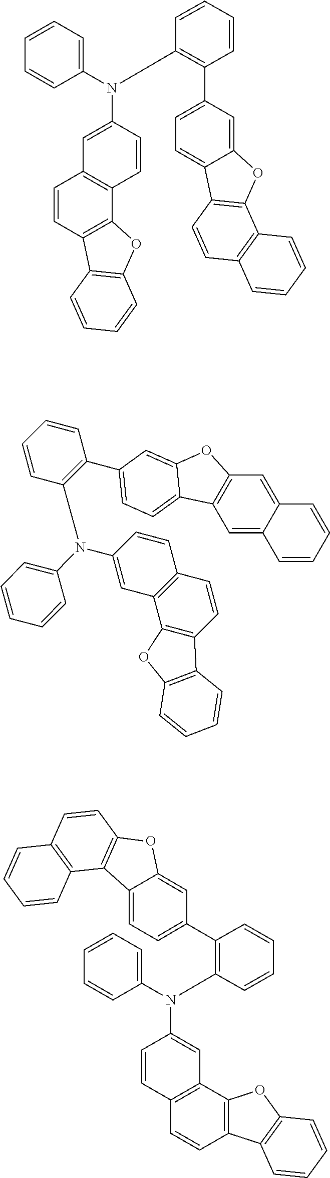 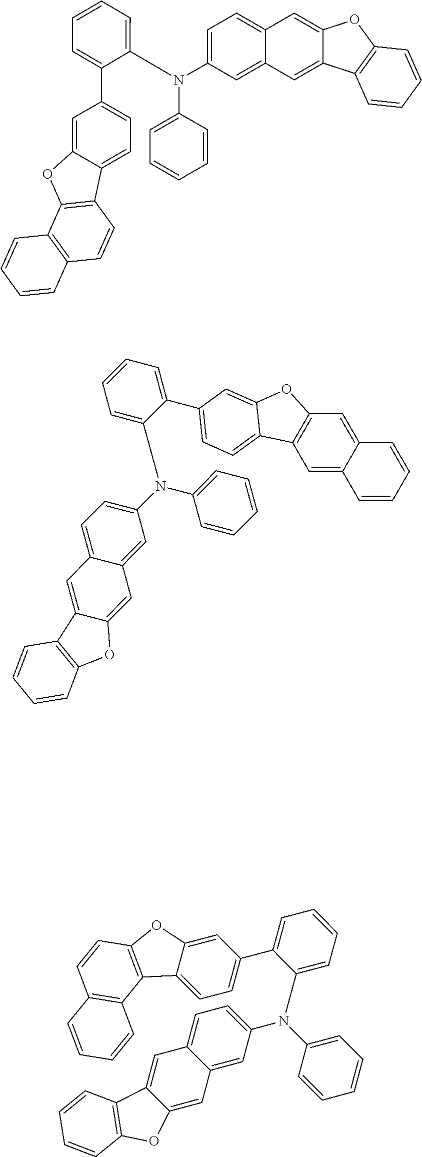 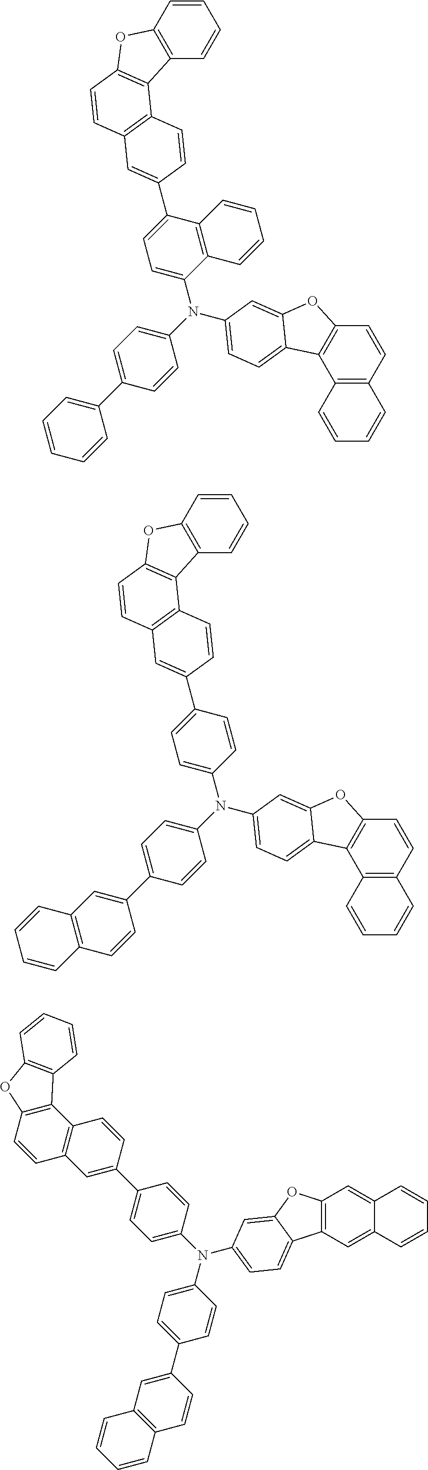 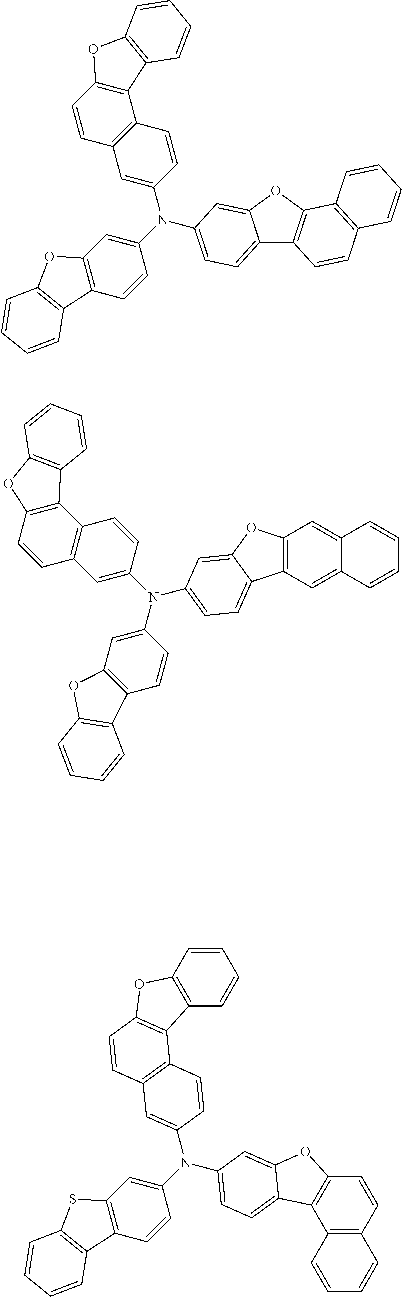 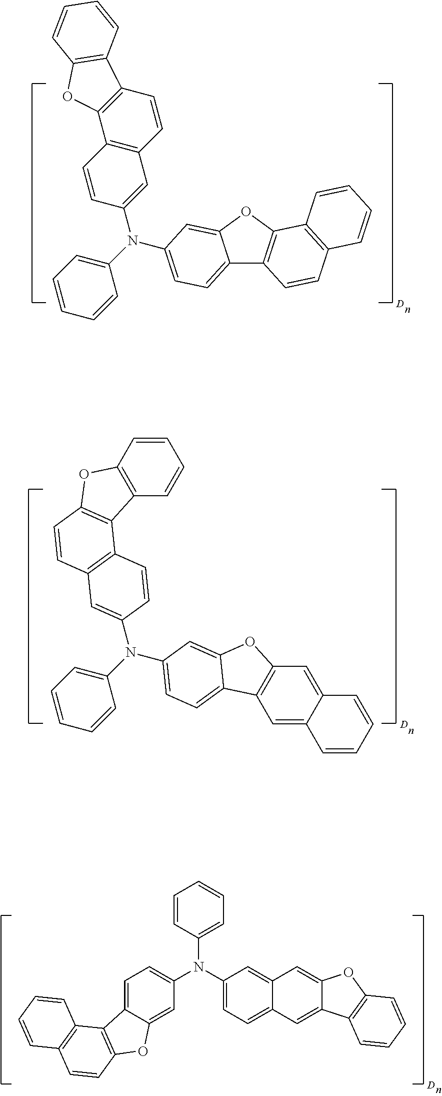 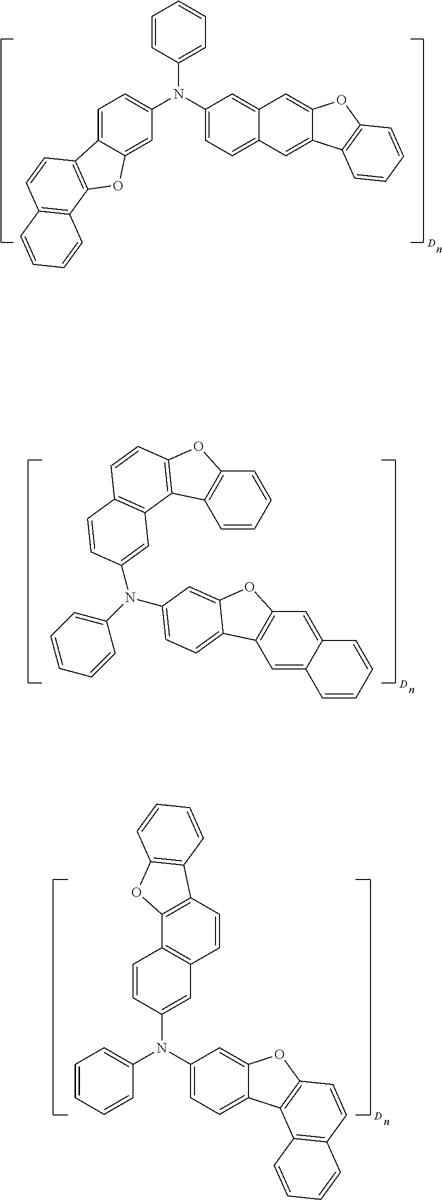 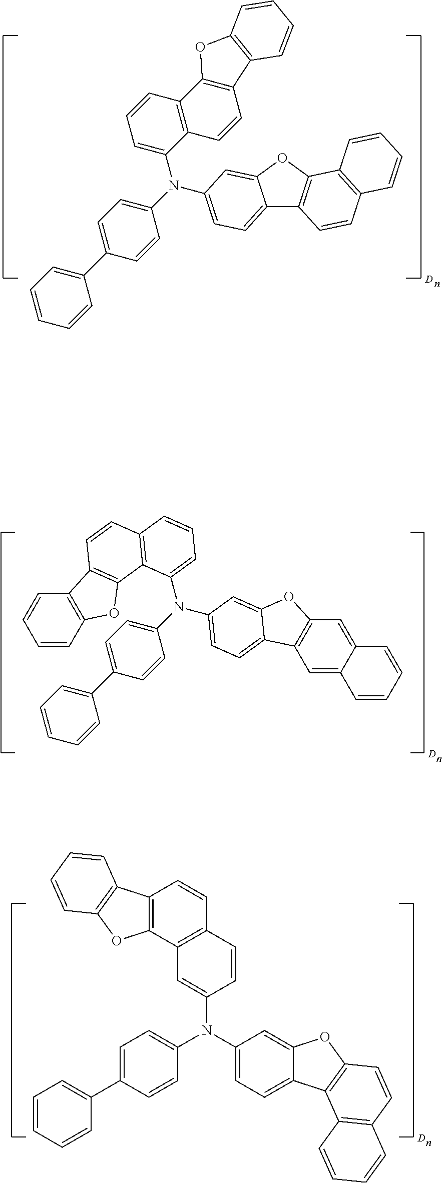 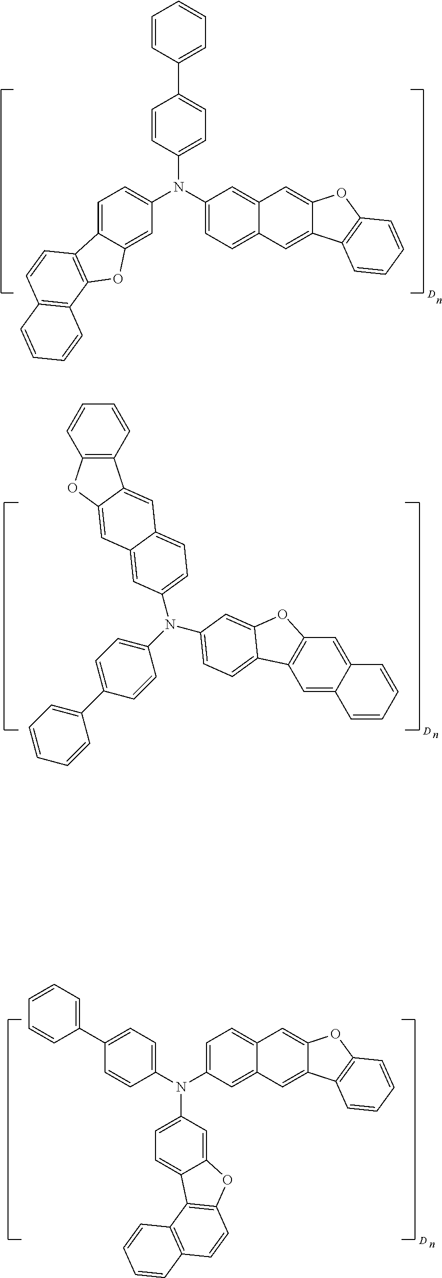 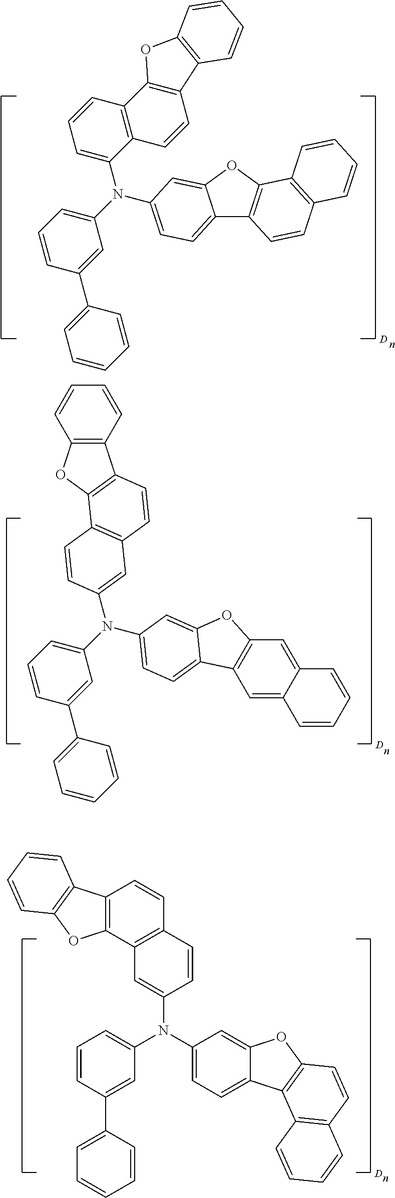 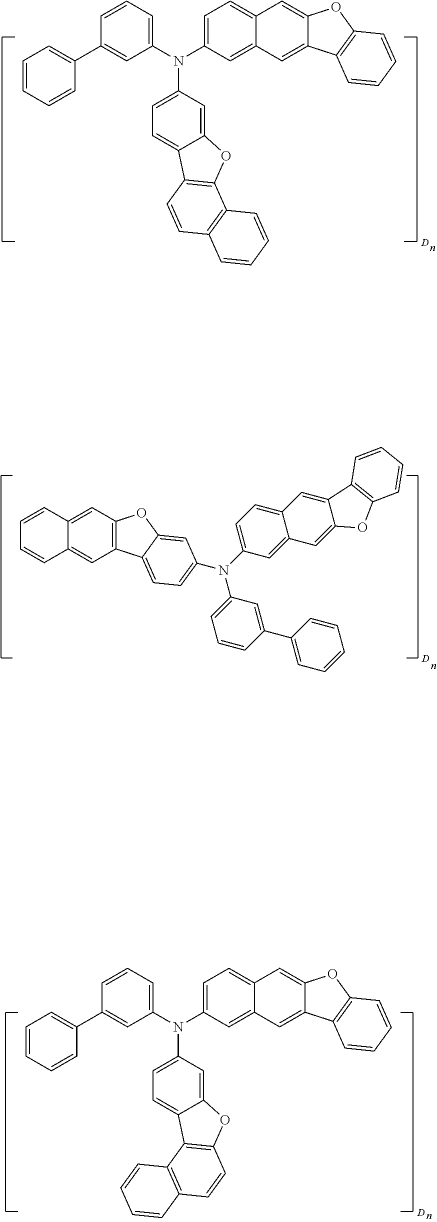 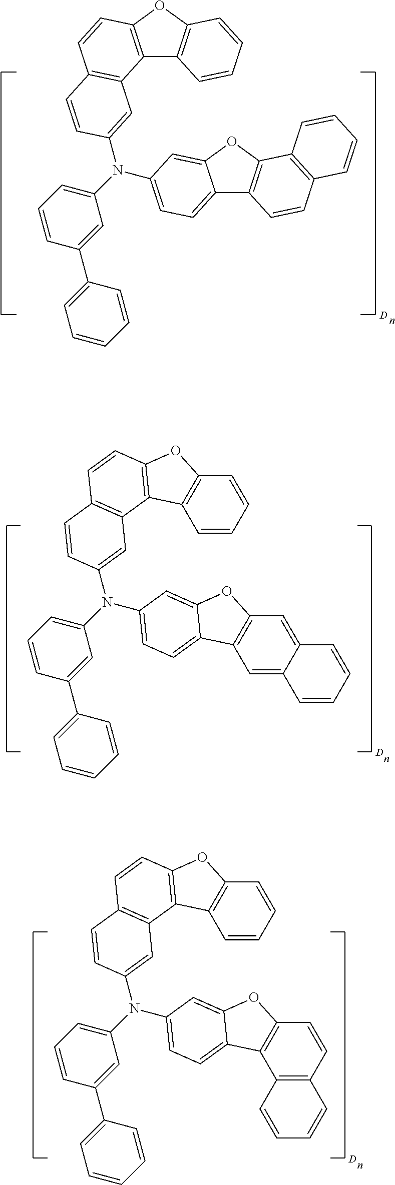 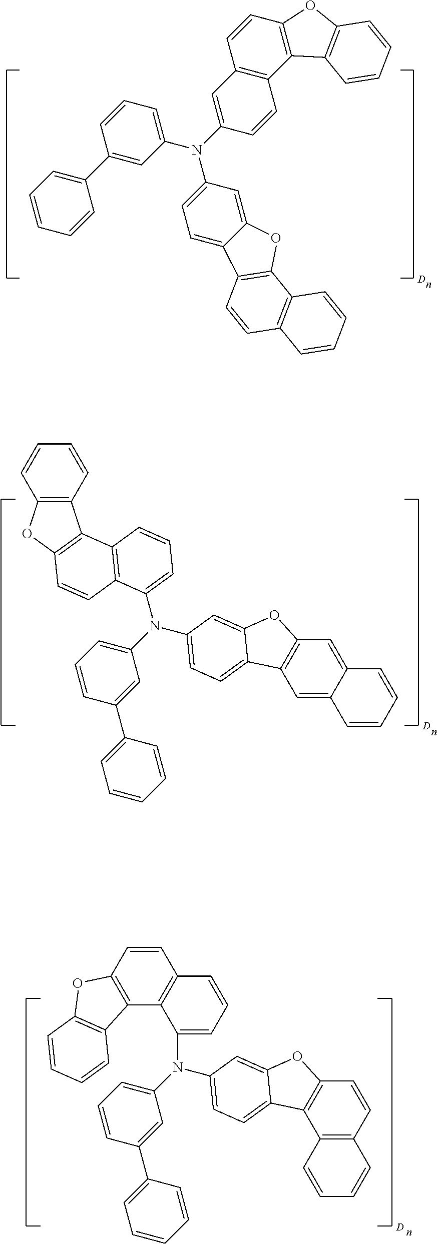 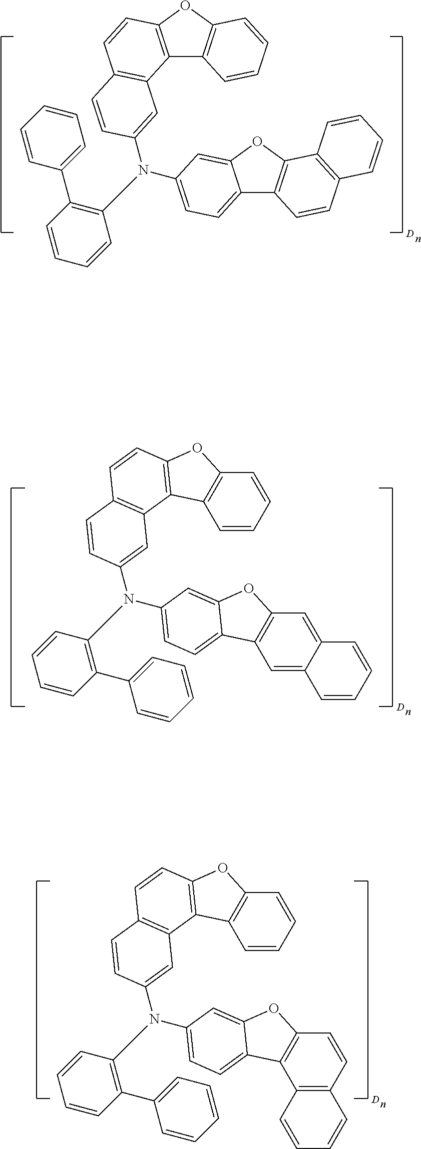 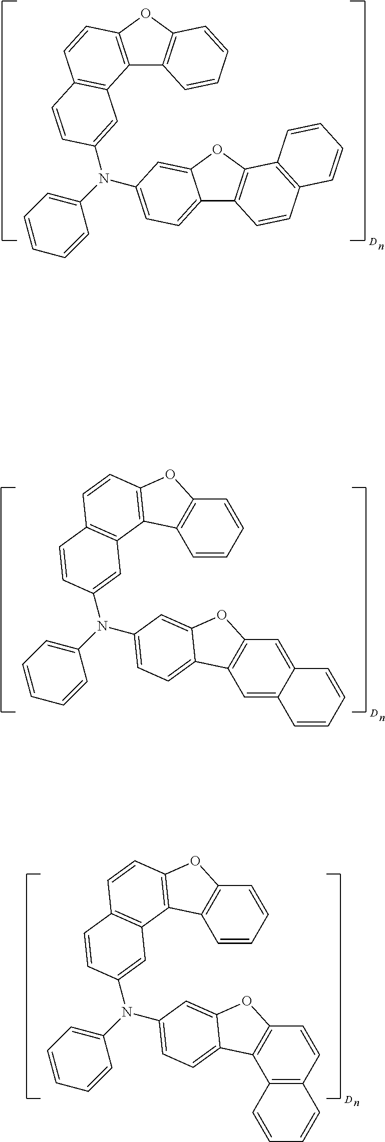 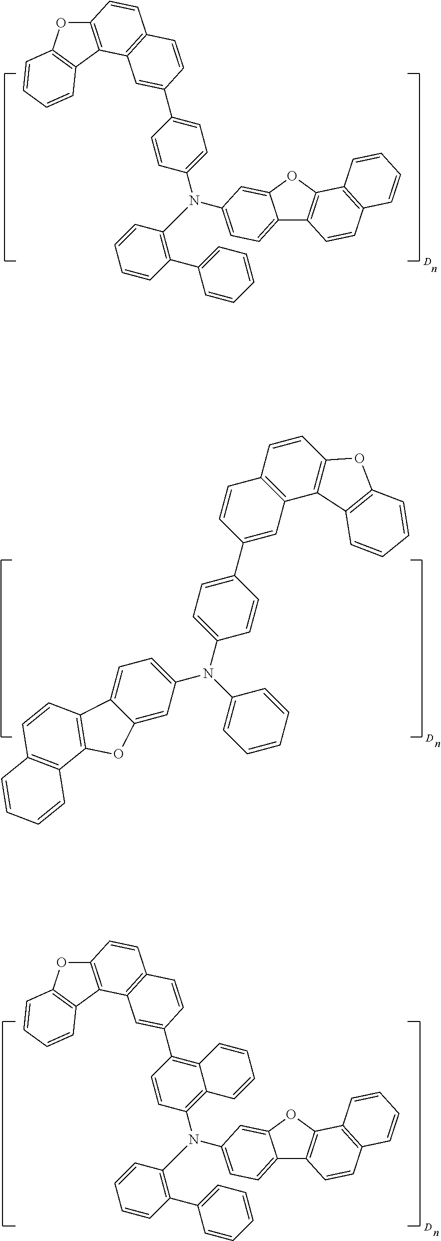 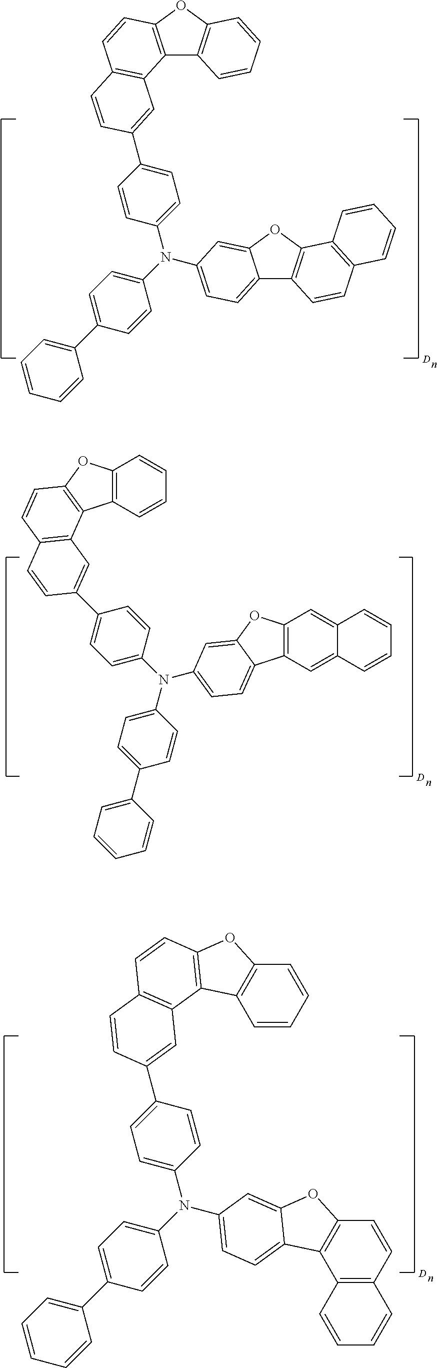 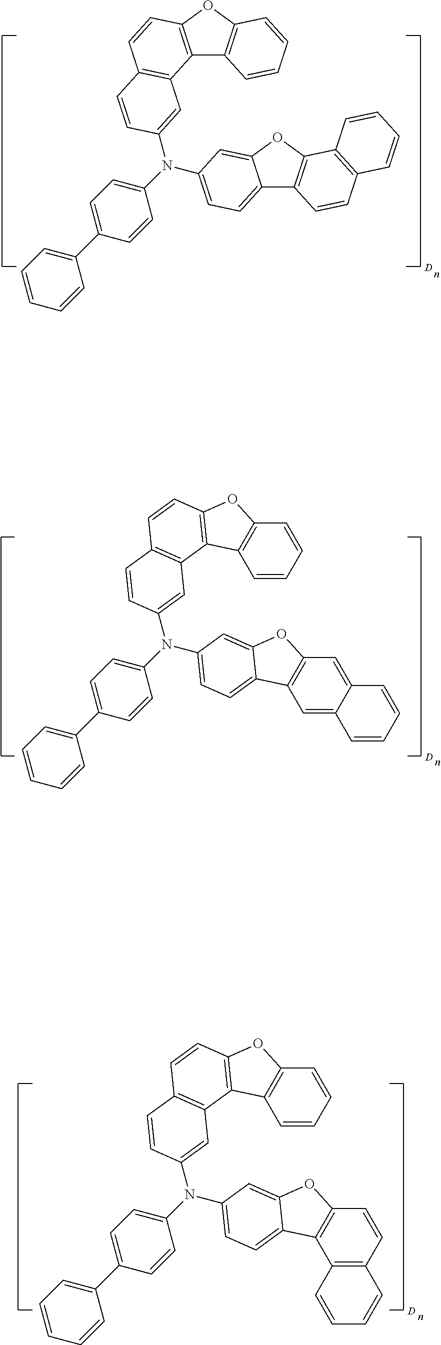 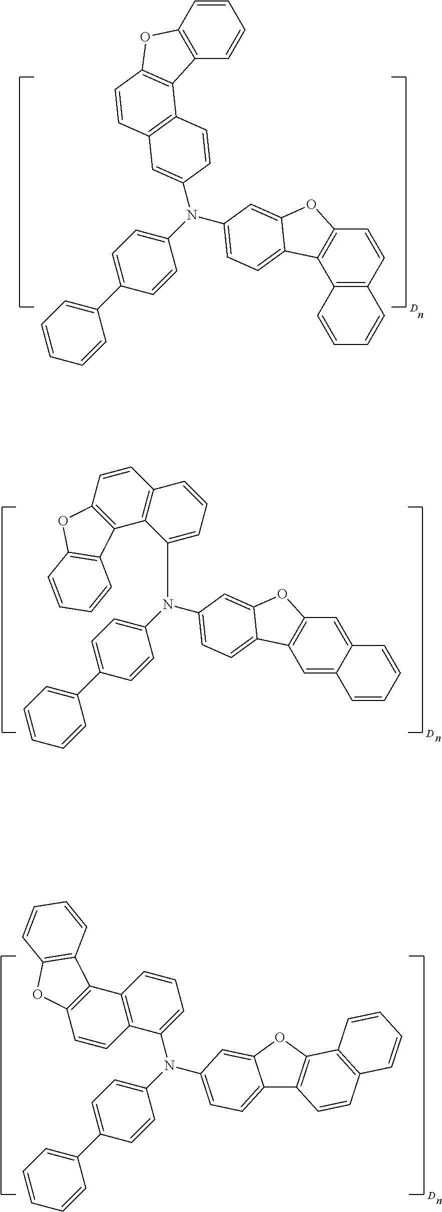 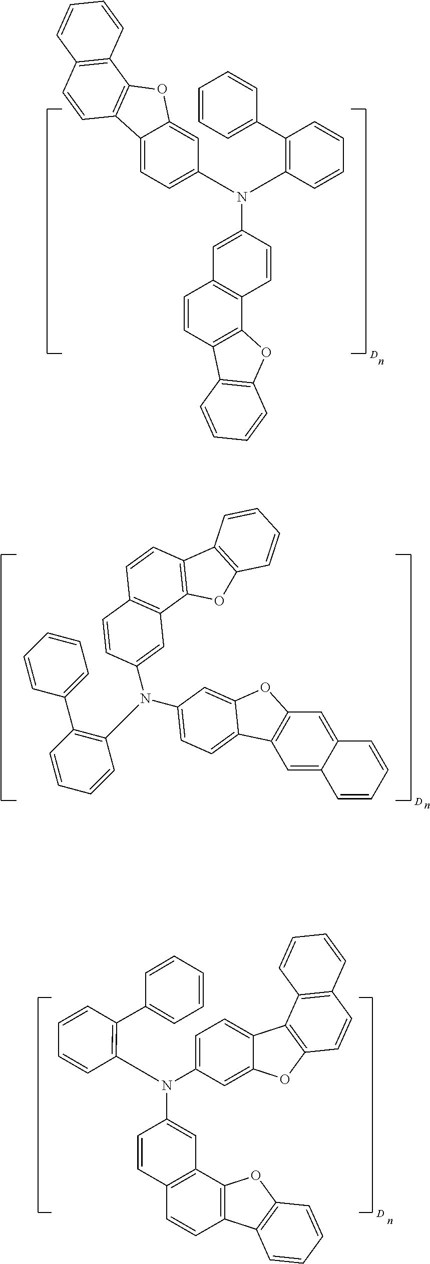 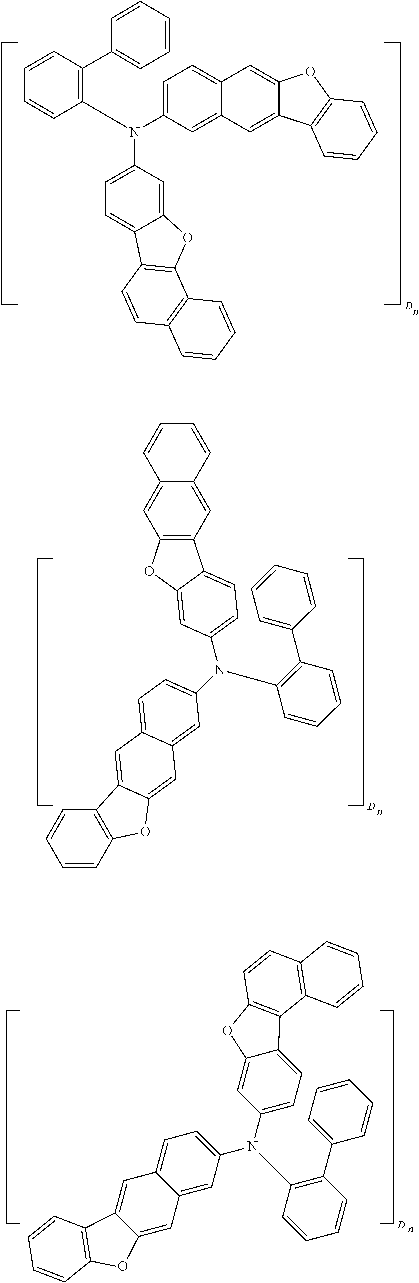 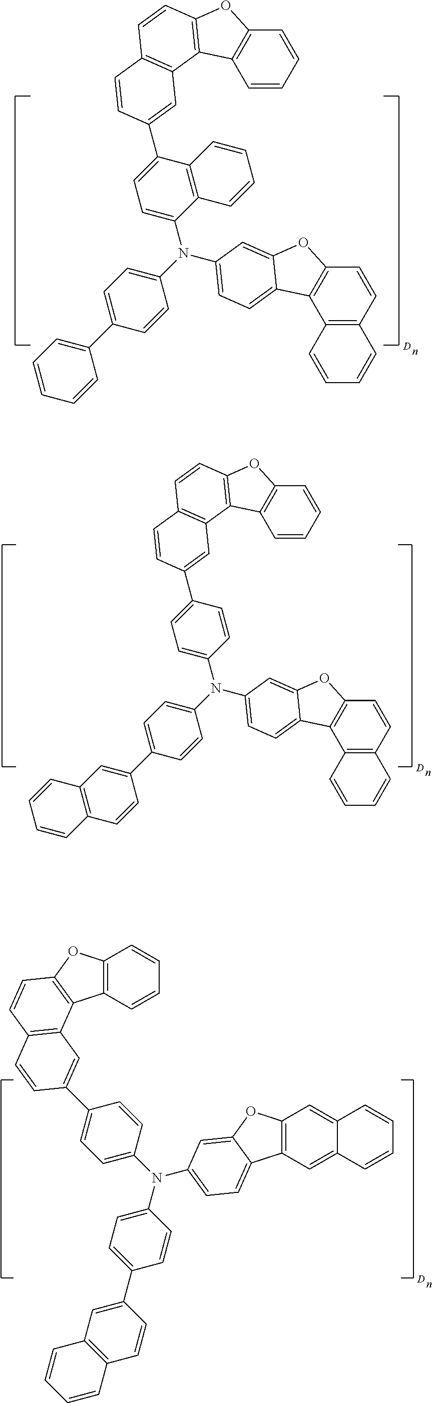 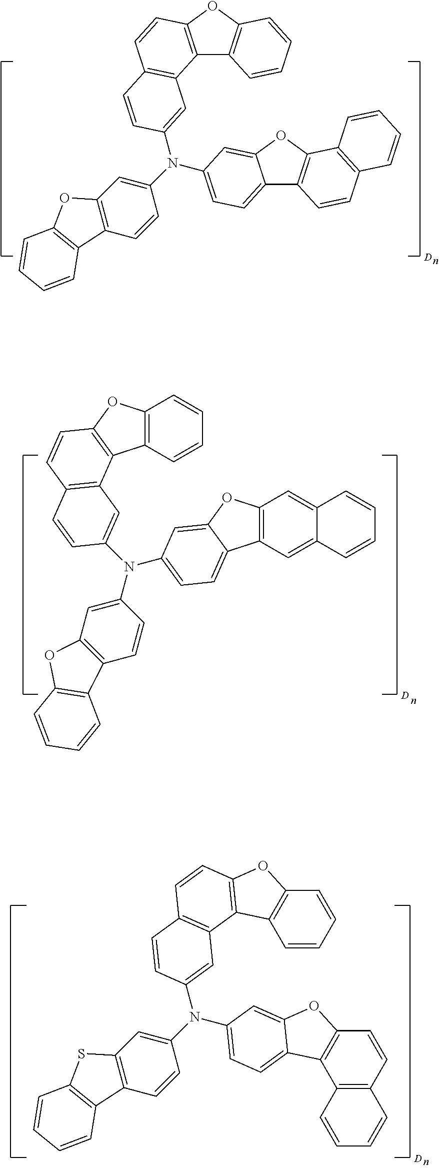 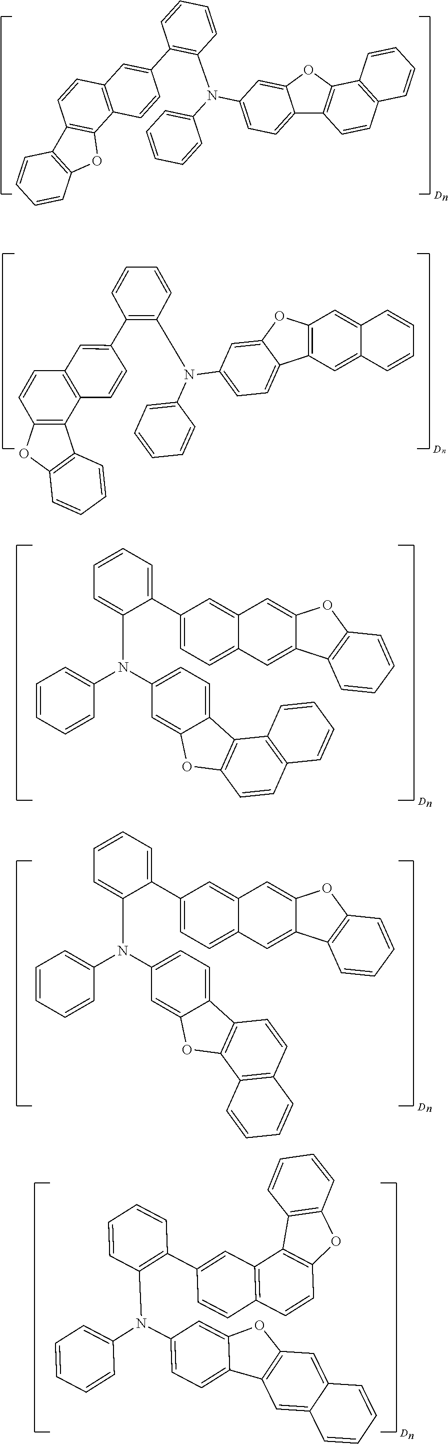 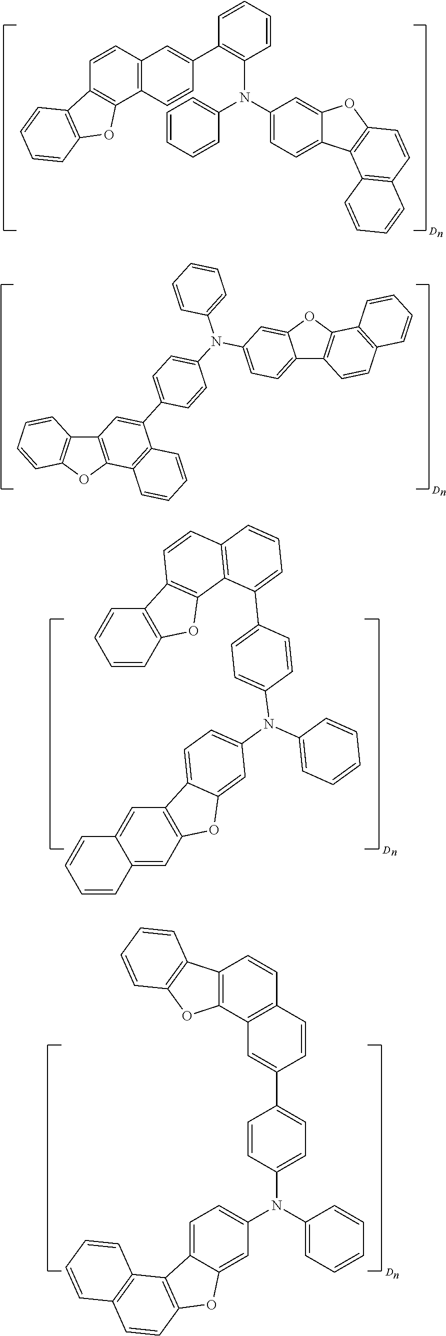 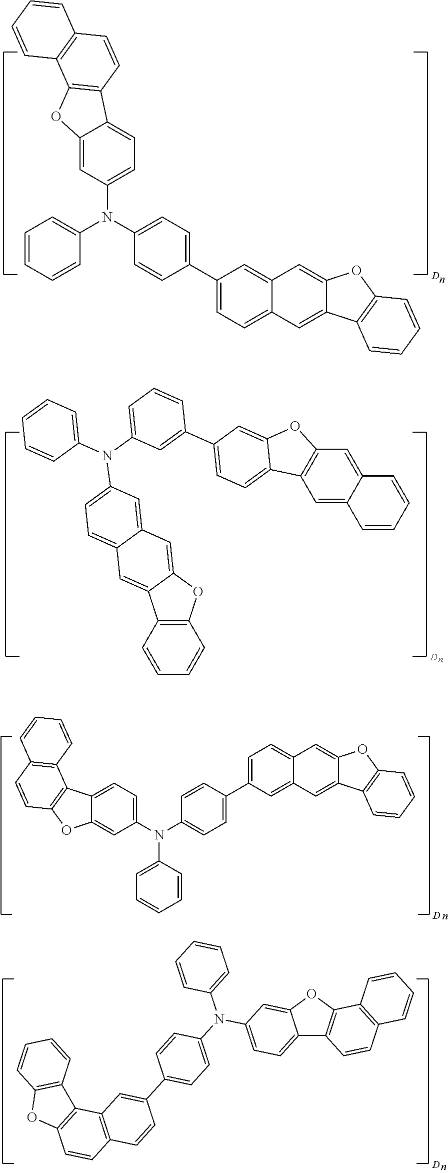 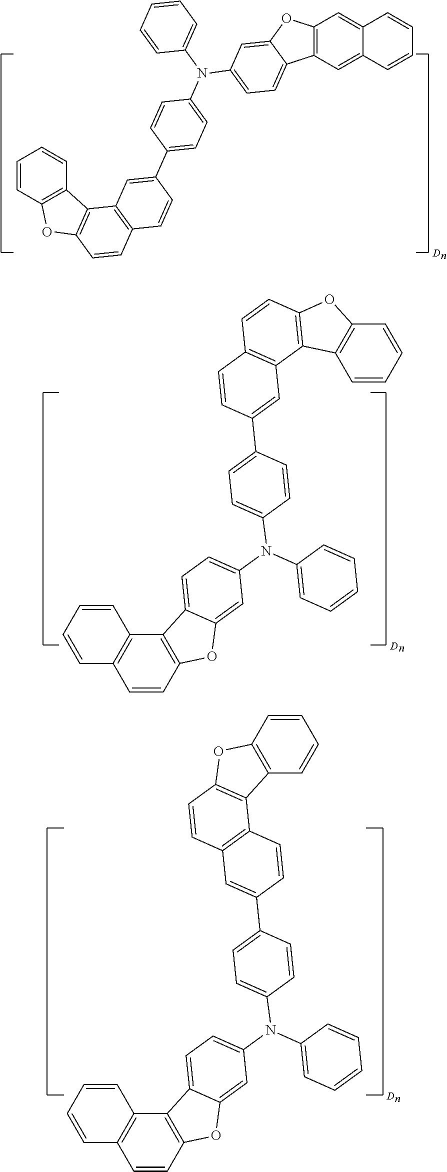 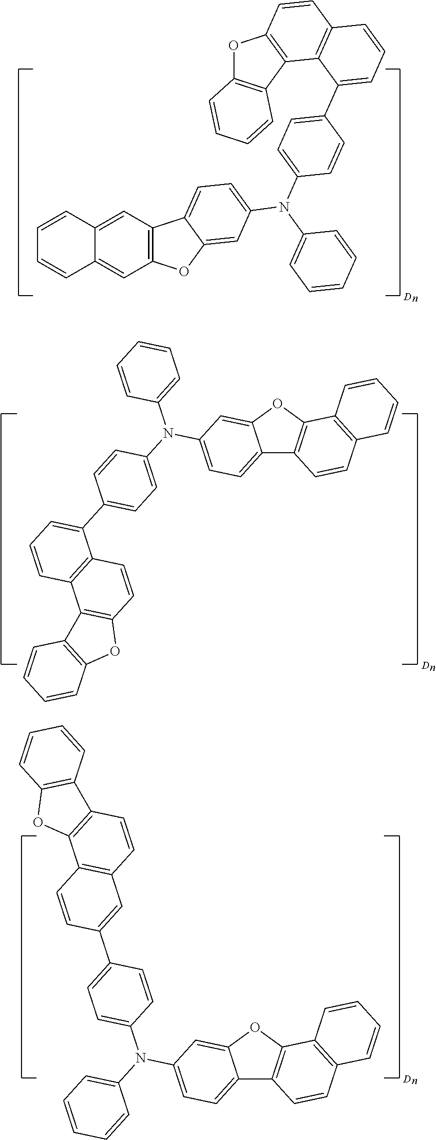 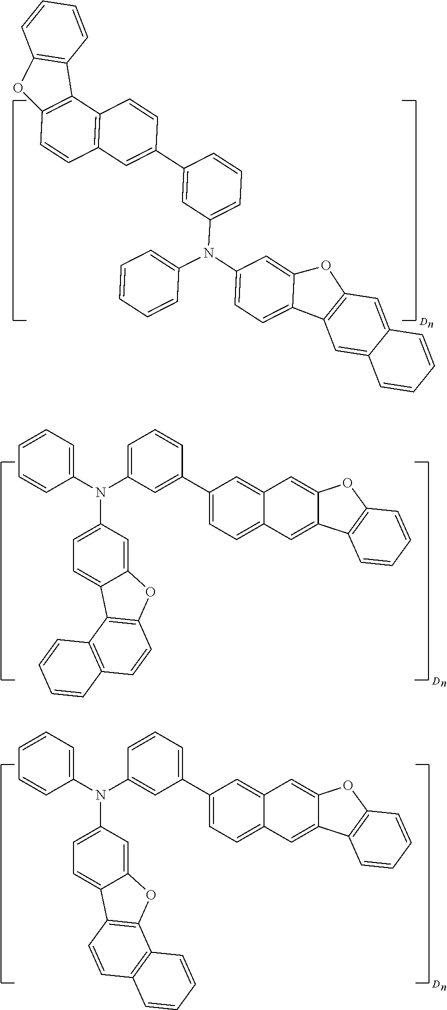 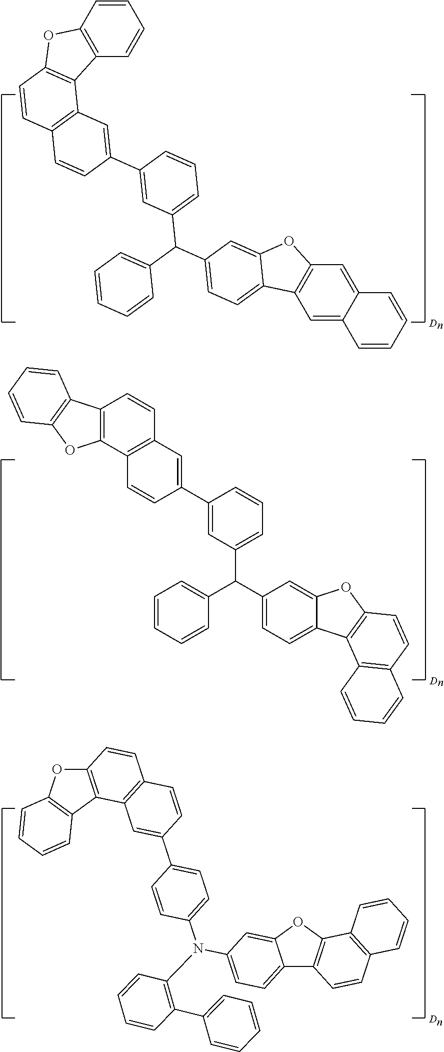 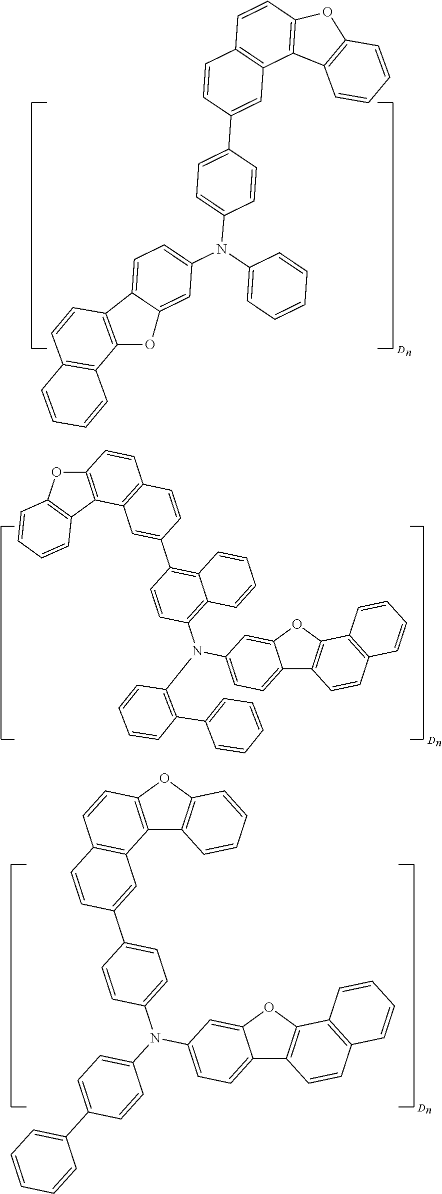 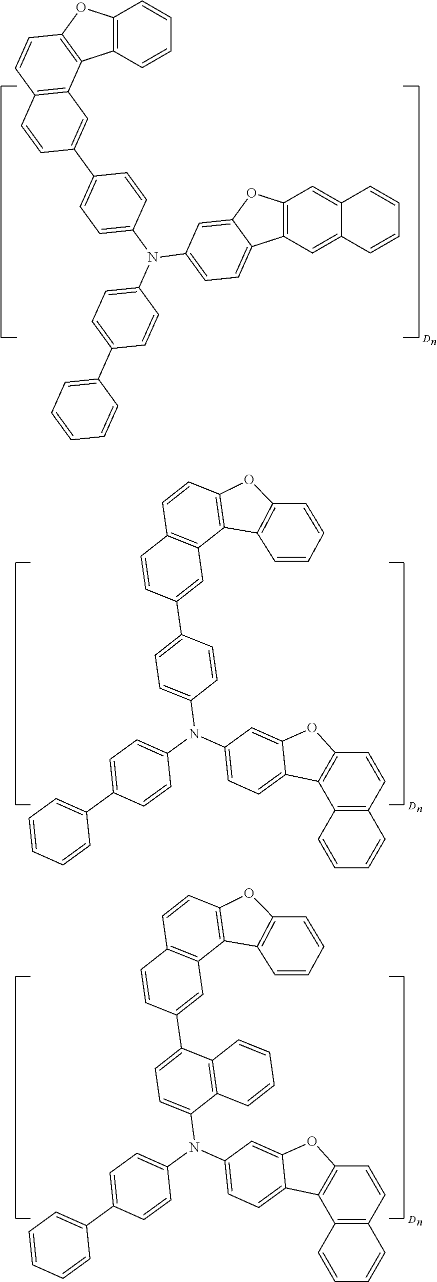 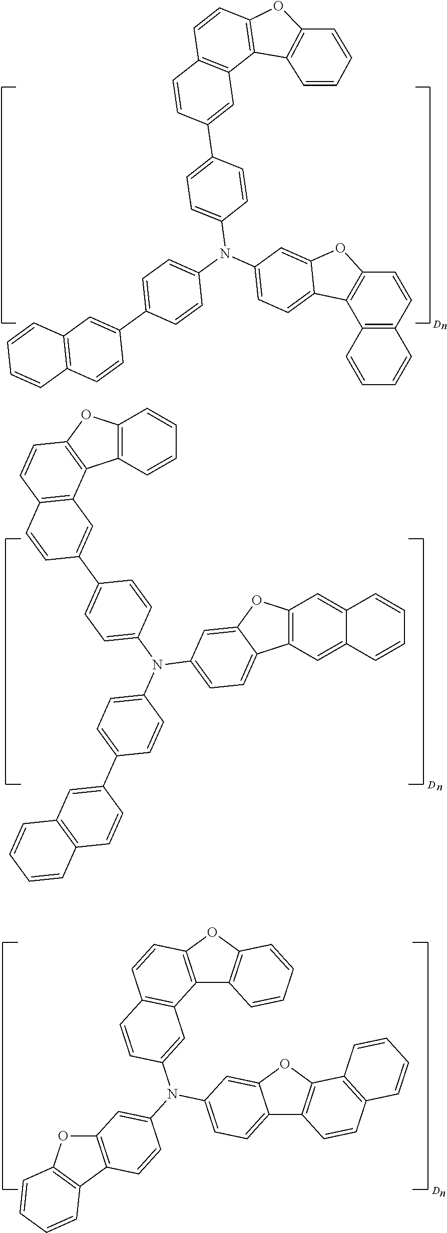 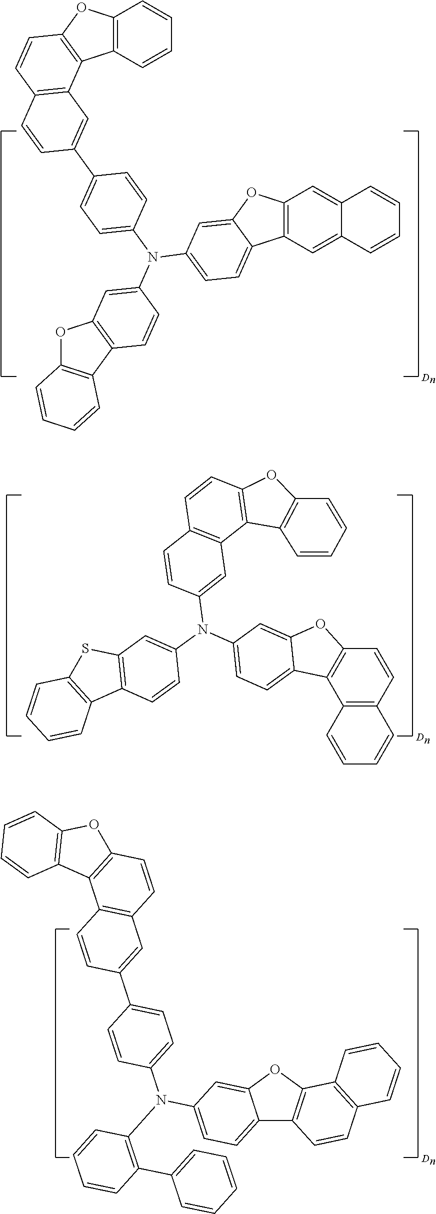 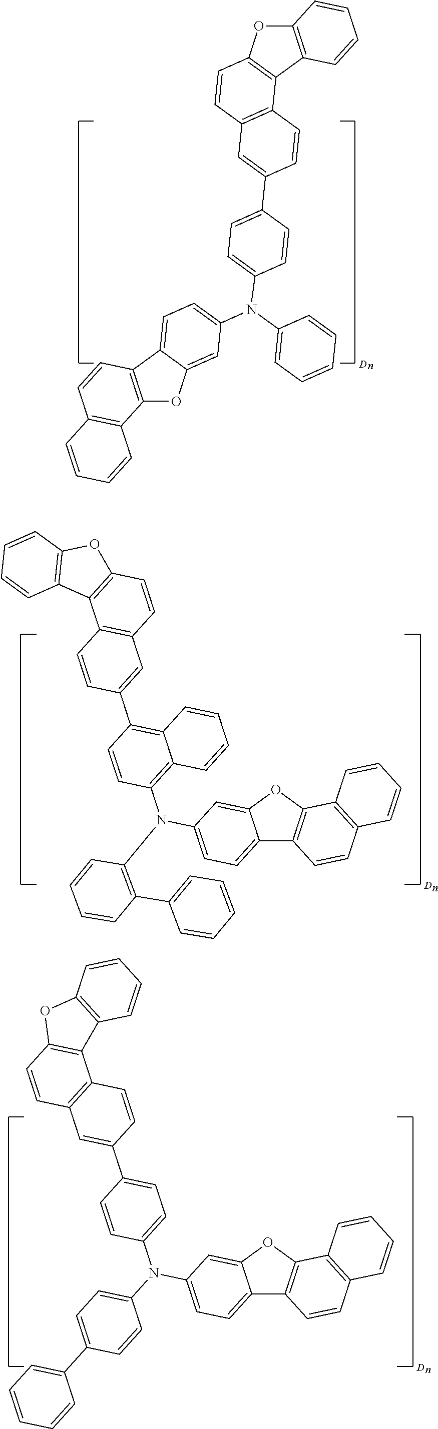 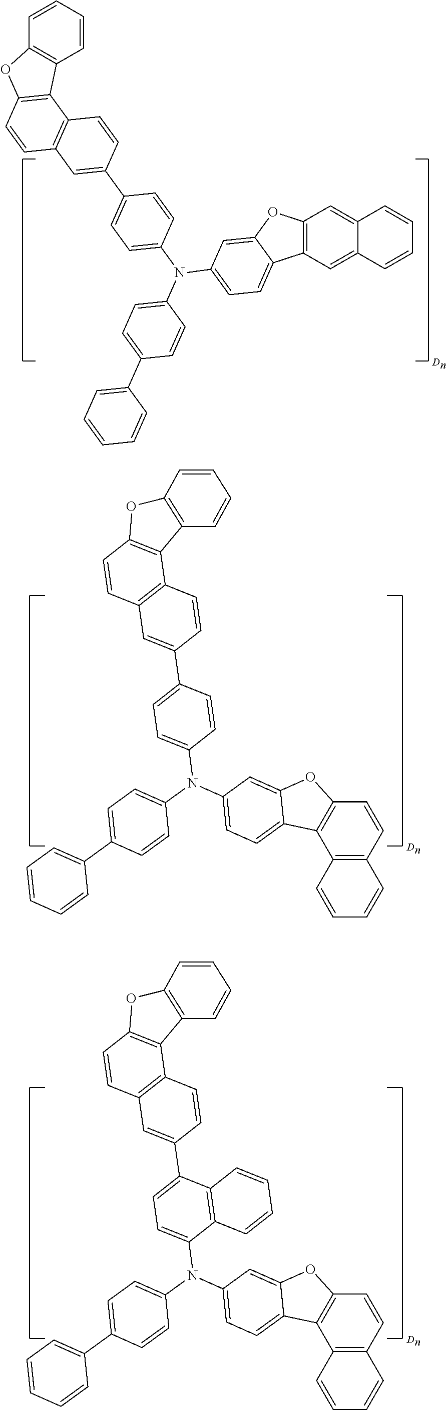 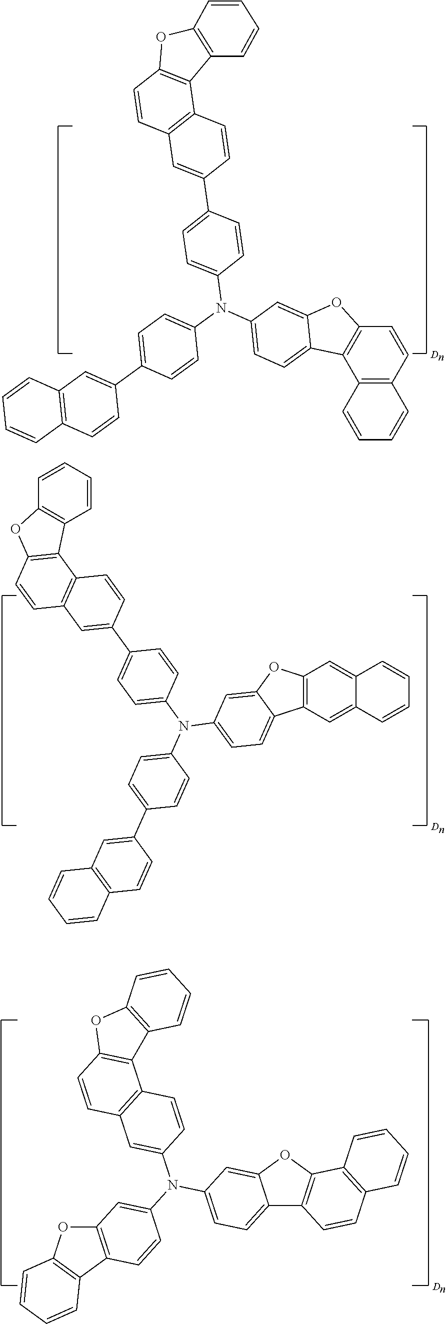 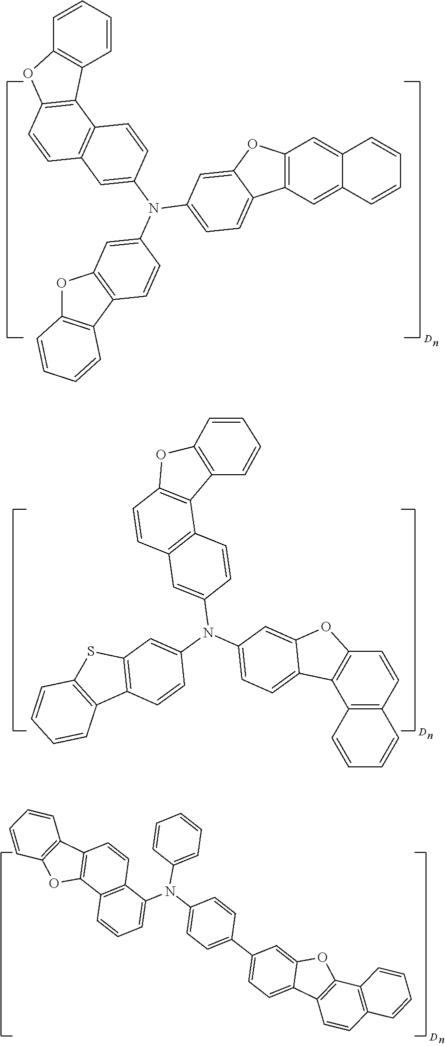 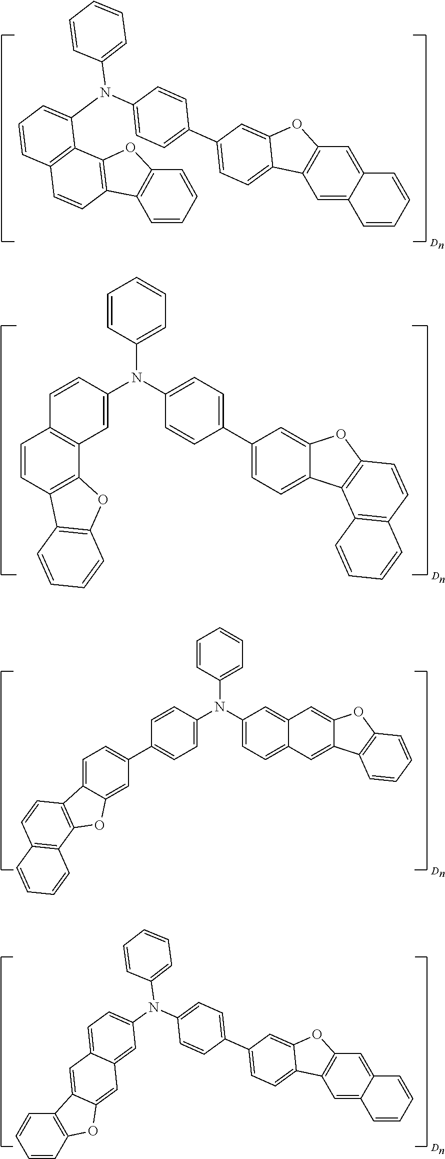 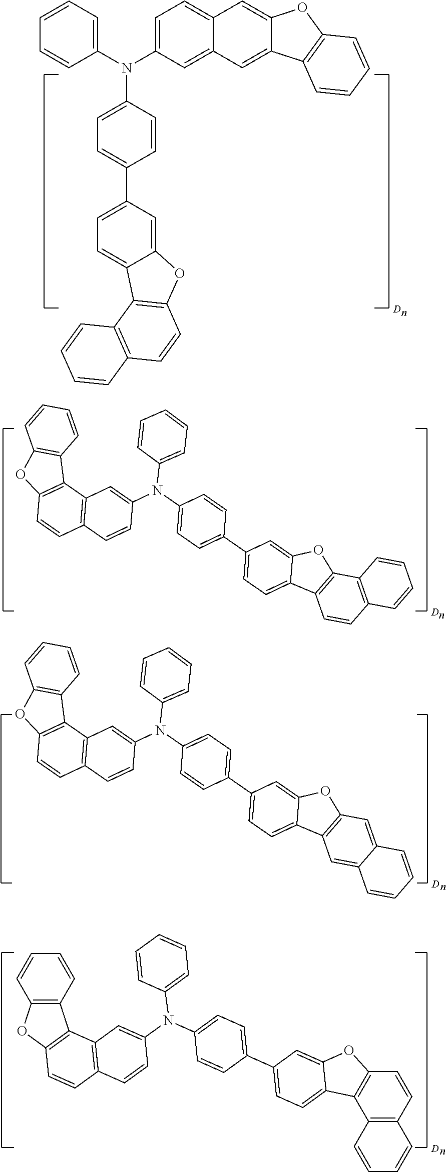 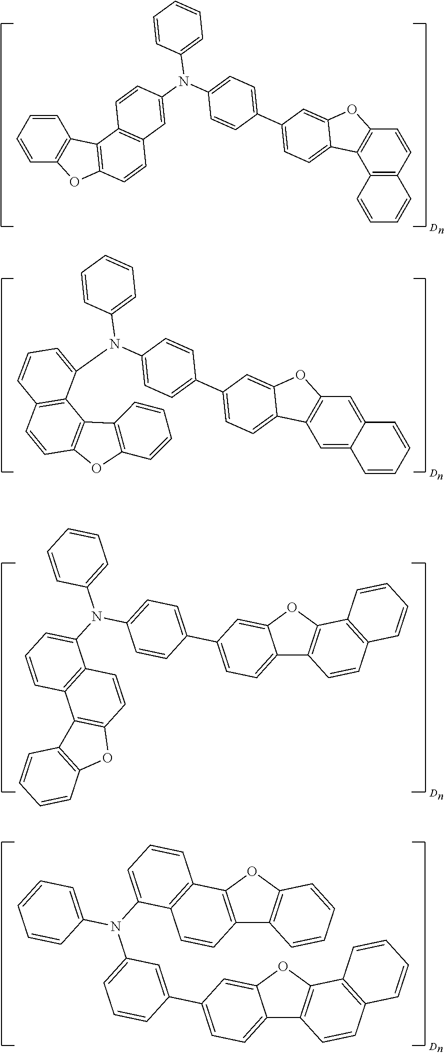 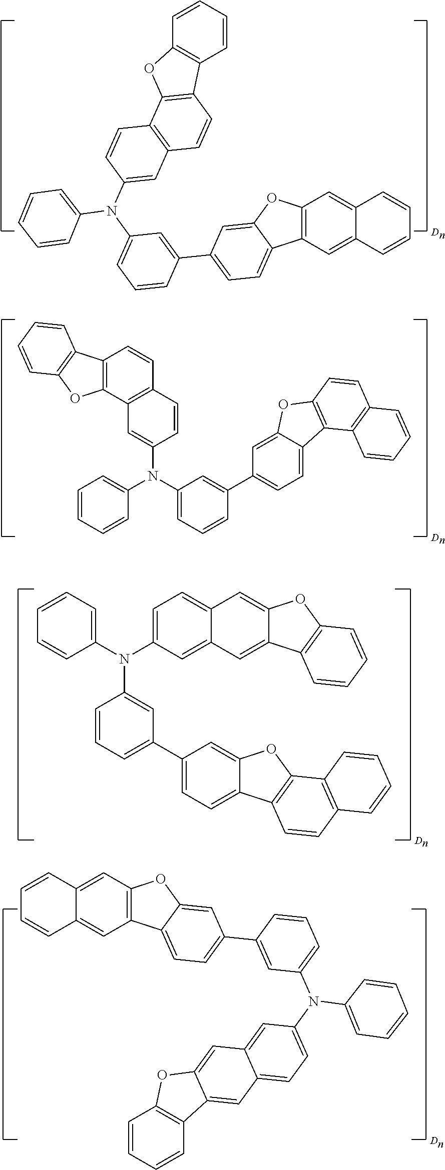 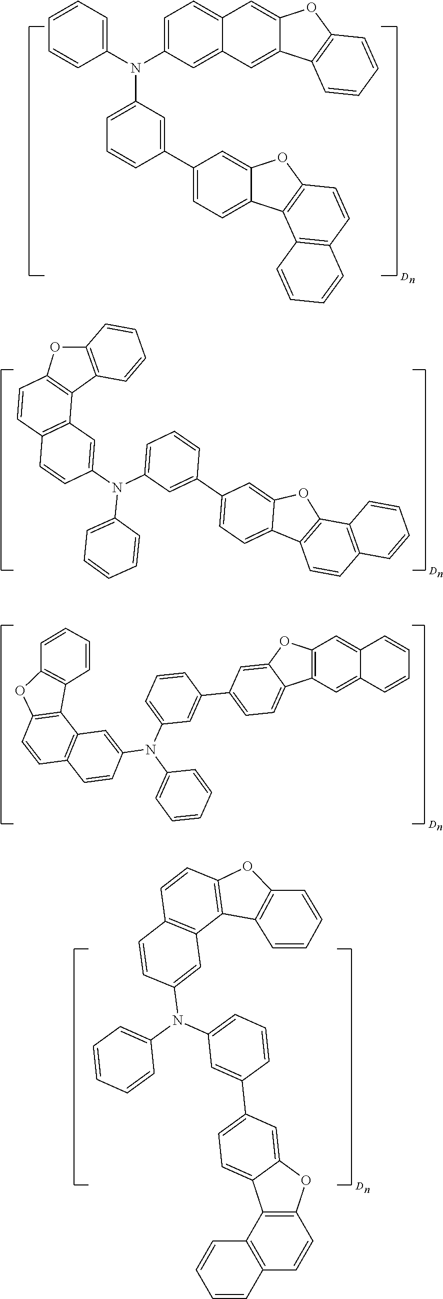 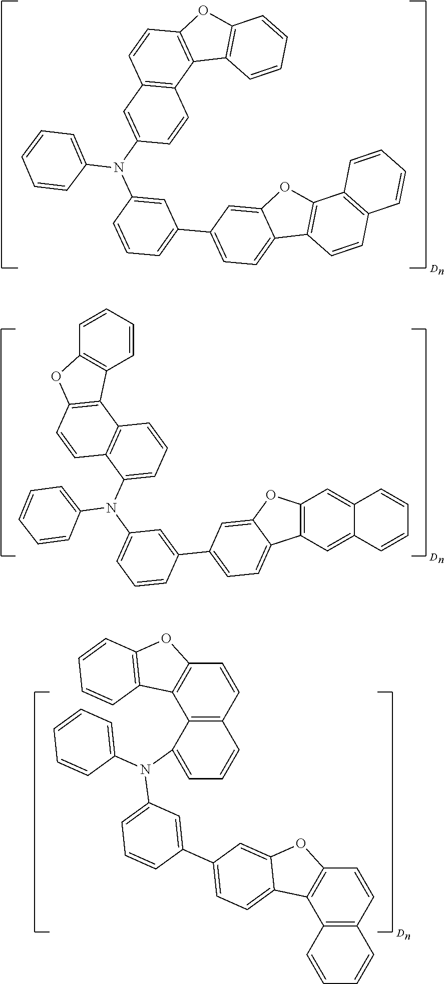 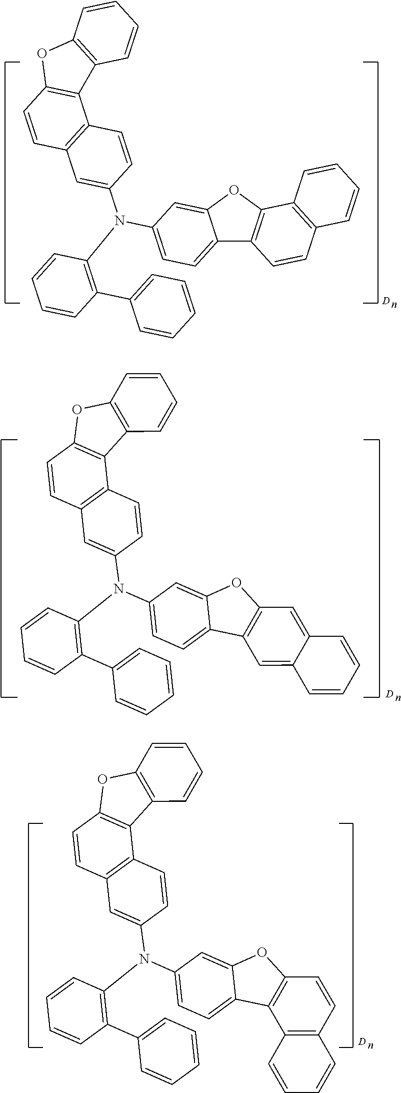 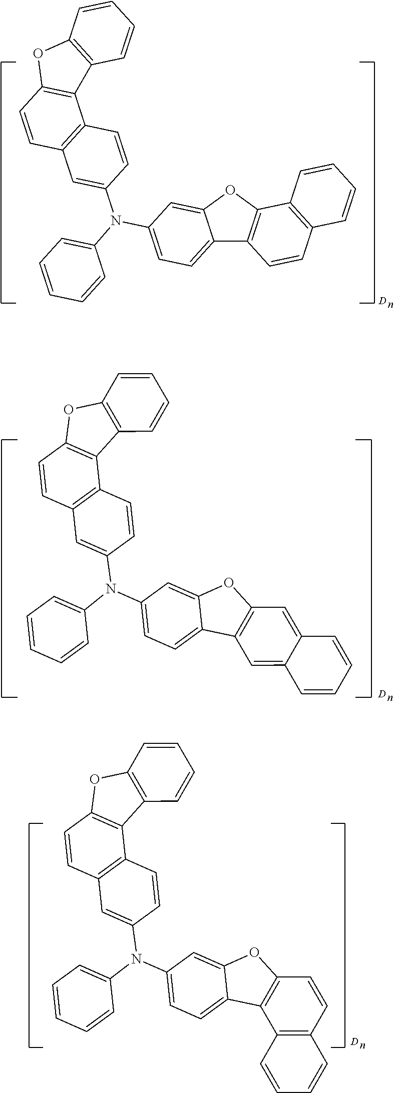 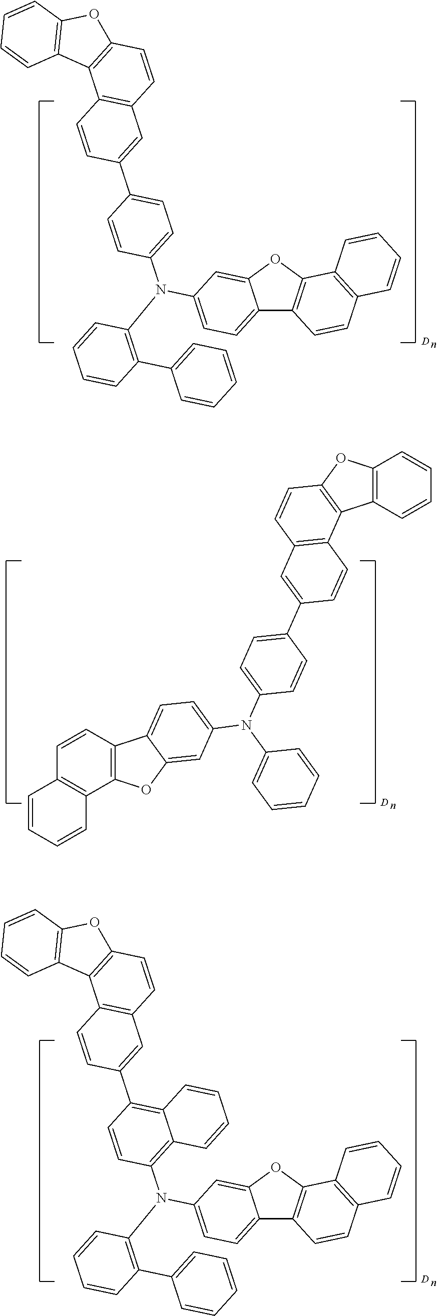 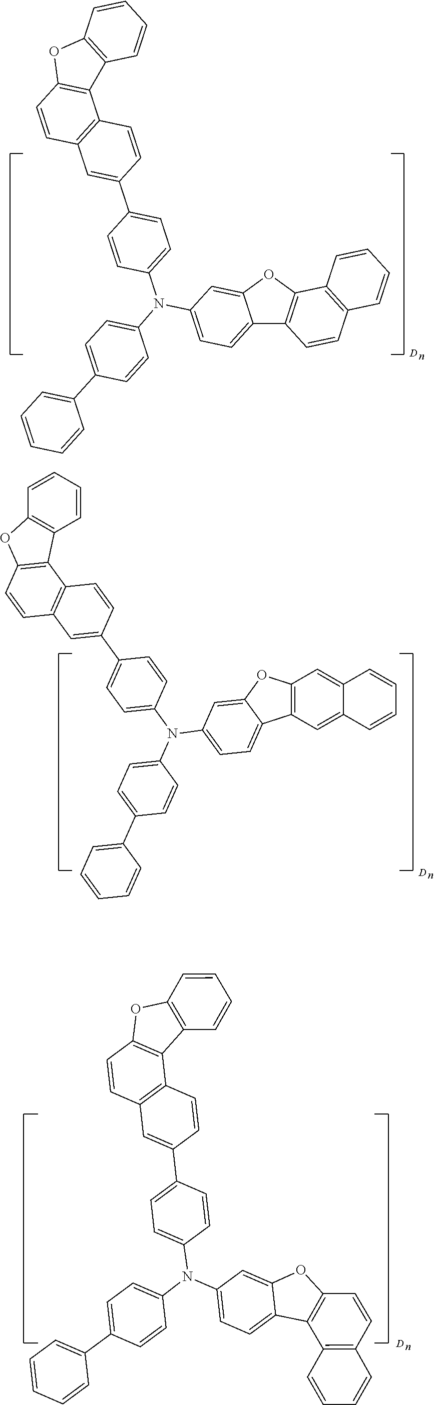 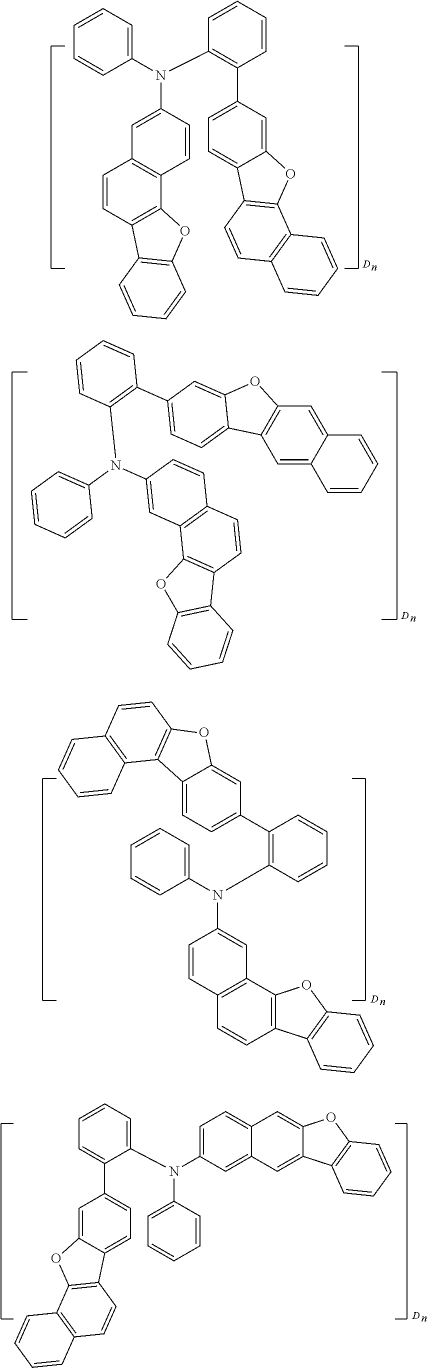 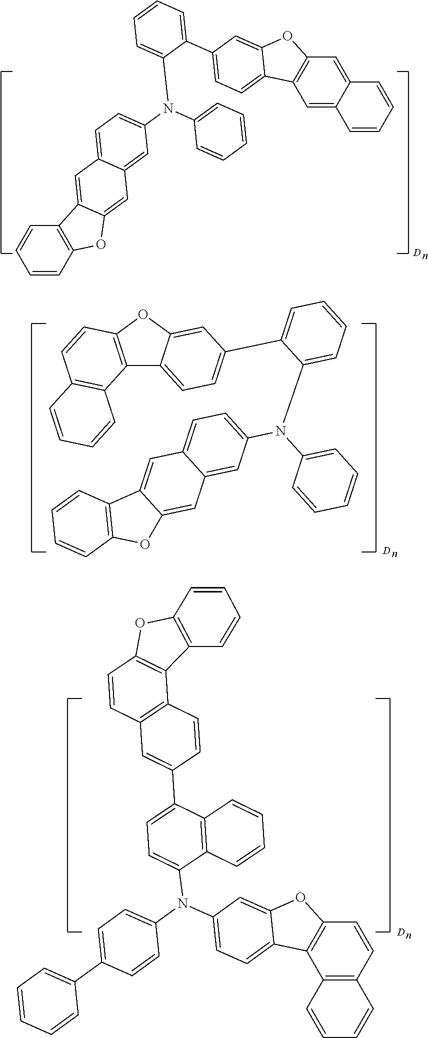 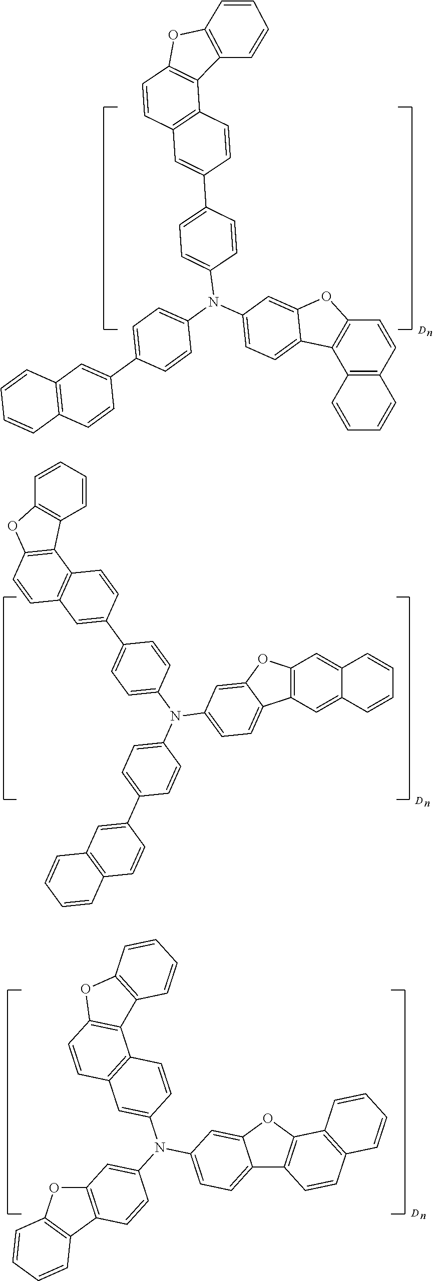 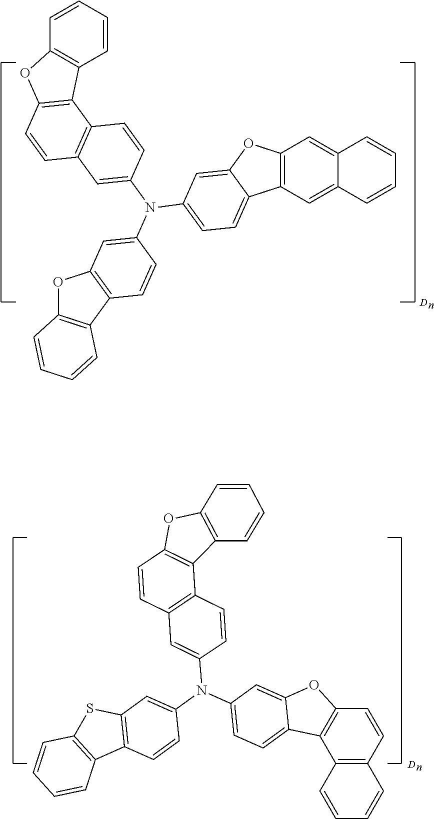
wherein:
Dn means that n hydrogens have been replaced by deuterium,
wherein n is an integer of 1 or more.
